(19) United States
(12) Patent Application Publication
(10) Pub. No.: US 2024/0067640 A1
(43) Pub. Date: Feb. 29, 2024

(54) PHENYL MALEIMIDE LINKER AGENTS

(71) Applicant: FIREFLY BIO, INC., South San Francisco, CA (US)

(72) Inventors: Sean Wesley Smith, Seattle, WA (US); Scott Allan Mitchell, Seattle, WA (US); Jack Chang Hung Lee, Seattle, WA (US)

(21) Appl. No.: 18/182,251

(22) Filed: Mar. 10, 2023

Related U.S. Application Data
(60) Provisional application No. 63/319,197, filed on Mar. 11, 2022.
(60) Provisional application No. 63/391,210, filed on Jul. 21, 2022.

(51) Int. Cl. C07D 471/04; C07H 7/06; C07F 9/6561; C07D 491/22; C07J 71/00; A61K 47/54

(52) U.S. Cl. CPC C07D 471/04; C07H 7/06; C07F 9/65616; C07D 491/22; C07J 71/0005; A61K 47/547

(57) ABSTRACT

Compounds useful as linker-payload compounds are disclosed. The compounds have the following Structure (I):

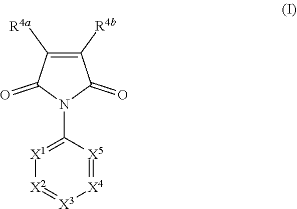

as a stereoisomer, enantiomer or tautomer thereof or a mixture thereof; or a pharmaceutically acceptable salt, solvate or prodrug thereof, wherein X¹, X², X³, X⁴, X⁵, R^{4a}, and R^{4b} are as defined herein. Additional compounds, conjugates, methods of preparation, pharmaceutical compositions, and methods of treatment related to conjugates of compounds of Structure (I) and a targeting moiety, or binding fragment thereof, are also provided.

### BACKGROUND

Antibody-drug conjugates (ADCs) are a class of bio-conjugates that are of interest. ADCs for certain cancer treatments combine the targeting features of monoclonal antibodies with cancer-killing ability of cytotoxic drugs to provide several advantages over other non-targeted chemotherapeutics. However, challenges related to the complexity of ADC molecules, specifically the linker between targeting peptide and cytotoxic drug, has caused significant complications for progress and development of new and effective therapeutics. Although the first ADC was approved in 2001, it took almost a decade before the next ADC was approved. Next generation ADC molecules now include non-cytotoxic payloads, such as immune-oncology drugs to stimulate the innate and adaptive immune systems to attack a tumor from within.

There exists a need for chemical linkers capable of enhancing the effectiveness of targeted therapeutic molecules (e.g., ADCs). The present disclosure fulfills this need and provides further related advantages.

### BRIEF DESCRIPTION OF THE DRAWINGS

In the figures, identical reference numbers identify similar elements (e.g., conjugates of Table 2). The sizes and relative positions of elements in the figures are not necessarily drawn to scale. For example, the shapes of various elements and angles are not drawn to scale and some of these elements are enlarged and positioned to improve figure legibility. Further, the particular shapes of the elements as drawn, are not intended to convey any information regarding the actual shape of the particular elements and have been solely selected for ease of recognition in the figures.

FIG. 1A shows TNF-α production from human PBMCs induced by conjugate II-12 in a dose-dependent manner in the presence of the HER2 expressing SK-BR-3 tumor cell line.

FIG. 1B shows a lack of TNF-α production from human PBMCs induced by that conjugate II-12 in the presence of the non-HER2 expressing MDA-MB-48 cell line.

FIG. 2A shows TNF-α production from human PBMCs induced by conjugates II-15, II-1, II-16, and II-17 in a dose-dependent manner in the presence of a Nectin-4 expressing MDA-MB-175-VII tumor cell line.

FIG. 2B shows a lack of TNF-α production from human PBMCs induced by conjugates II-15, II-1, II-16, and II-17 in the presence of HEK-293 cells lacking expression of Nectin-4.

FIG. 3A shows TNF-α production from human PBMCs induced by conjugates II-18, II-19, II-19, II-20, II-21 in a dose-dependent manner in the presence of a Nectin-4 expressing MDA-MB-175-VII tumor cell line.

FIG. 3B shows a lack of TNF-α production from human PBMCs induced by conjugates II-18, II-19, II-19, II-20, II-21 in the presence of HEK-293 cells lacking expression of Nectin-4.

FIG. 4A shows TNF-α production from human PBMCs induced by conjugate II-3 in a dose-dependent manner in the presence of a Nectin-4 expressing MDA-MB-175-VII tumor cell line.

FIG. 4B shows a lack of TNF-α production from human PBMCs induced by conjugate II-3 in the presence of HEK-293 cells lacking expression of Nectin-4.

FIG. 5A shows TNF-α production from human PBMCs induced by conjugate II-2 in a dose-dependent manner in the presence of a Nectin-4 expressing MDA-MB-175-VII tumor cell line.

FIG. 5B shows a lack of TNF-α production from human PBMCs induced by conjugate II-2 in the presence of HEK-293 cells lacking expression of Nectin-4.

### DETAILED DESCRIPTION

The instant disclosure provides branched phenyl maleimide compounds (e.g., compounds of Structure (I)) to facilitate loading biologically active molecules (also referred to herein as payloads) onto targeting compounds, such as antibodies or ligands. The instant disclosure further provides use of linker compounds to make a linker-payload for conjugation to a targeting moiety (e.g., antibody, fusion protein, ligand). An advantage of using a hydrophilic moiety (e.g., polar, hydrophilic, charged functional groups, etc.) in combination with a phenyl maleimide via a parallel to a payload topology is to increase stability when part of a conjugate and to minimize off-target uptake or activity by concealing or covering payload hydrophobicity as compared to when connected in series (or linearly) to a payload. Furthermore, such a structure appears to maximize the likelihood the drug-to-antibody ratio (DAR) will generally remain fixed while the conjugate is present in systemic circulation (that is, conjugate stability will be maintained while preserving favorable pharmacokinetic properties). For example, upon conjugate addition of a thiol present in cysteine to a compound of Structure (I), a thioether substituted succinimide is formed. While not wishing to be bound by theory, it is believed this succinimide hydrolyzes rapidly due to the aforementioned substituted phenyl group of Structure (I) and this hydrolyzed succinimide is no longer able to de-conjugate via a retro-Michael reaction. Thus, the linker-payload is ‘locked’ to the protein and tends to better preserve DAR.

Prior to setting forth this disclosure in more detail, it may be helpful to provide definitions of certain terms to be used herein. Additional definitions are set forth throughout this disclosure.

As used in the specification and claims, the singular form “a,” “an,” and “the” includes plural references unless the context clearly dictates otherwise. It should be understood that the terms “a” and “an” as used herein refer to “one or more” of the enumerated components.

The use of the alternative (e.g., “or”) should be understood to mean either one, both, or any combination thereof of the alternatives.

The term “about” as used herein in the context of a number refers to a range centered on that number and spanning 15% less than that number and 15% more than that number. The term “about” used in the context of a range refers to an extended range spanning 15% less than that the lowest number listed in the range and 15% more than the greatest number listed in the range. In some embodiments, a given value refers to a range of values (i.e., “about” the given value). For example, in some embodiments, pH 7.4 refers to about pH 7.4 (i.e., a range from 6.3 to 8.5).

Throughout this disclosure, any concentration range, percentage range, ratio range, or integer range is to be understood to include the value of any integer within the recited range and, when appropriate, fractions thereof (such as one tenth and one hundredth of an integer), unless otherwise indicated. Also, any number range of this disclosure relating to any physical feature, such as polymer subunits, size, or thickness, are to be understood to include any integer within the recited range, unless otherwise indicated. Throughout this disclosure, numerical ranges are inclusive of their recited endpoints, unless specifically stated otherwise.

Unless the context requires otherwise, throughout the present specification and claims, the word “comprise” and variations thereof, such as, “comprises” and “comprising” are to be construed in an open, inclusive sense, that is, as “including, but not limited to.” As used herein, the terms “include” and “comprise” are used synonymously.

The phrase “at least one of” when followed by a list of items or elements refers to an open-ended set of one or more of the elements in the list, which may, but does not necessarily, include more than one of the elements.

As used herein, a “variant” protein or polypeptide comprises one or more non-natural amino acids, one or more amino acid substitutions, one or more amino acid insertions, one or more amino acid deletions, or any combination thereof, which may occur at one or more sites relative to a reference polypeptide of this disclosure, and wherein the variant protein or polypeptide has substantially similar activity (e.g., enzymatic function, immunogenicity) relative to a reference polypeptide. A variant protein or polypeptide of this disclosure may have at least about 70%, 75%, 80%, 85%, 90%, 91%, 92%, 93%, 94%, 95%, 96%, 97%, 98%, 99%, or more sequence identity to the amino acid sequence for a reference polypeptide of this disclosure as determined by sequence alignment programs and parameters disclosed herein. A variant polypeptide can result from, for example, a genetic polymorphism or by human manipulation. Conservative substitutions of amino acids are well known and may occur naturally or may be engineered when a protein is recombinantly produced. Amino acid substitutions, deletions, and additions may be introduced into a protein using mutagenesis methods known in the art (see, e.g., Sambrook et al., Molecular Cloning: A Laboratory Manual, 3rd ed., Cold Spring Harbor Laboratory Press, NY, 2001). Oligonucleotide-directed site-specific (or segment specific) mutagenesis procedures may be employed to produce polynucleotides having altered codons that provide the desired substitution, deletion, or insertion. Alternatively, random or saturation mutagenesis techniques, such as alanine scanning mutagenesis, error prone polymerase chain reaction mutagenesis or oligonucleotide-directed mutagenesis, may be used to prepare polypeptide variants (see, e.g., Sambrook et al., supra).

In some embodiments, a “binding domain” or a “binding region” or “targeting moiety” refers to a protein, polypeptide, oligopeptide, peptide, carbohydrate, nucleic acid, or combination thereof that is capable of specifically binding to a target or multiple targets (e.g., Nectin-4, MSLN, HER2, LRRC15, ASGR1, CD40, or BAFF and/or APRIL). A binding domain includes any naturally occurring, synthetic, semi-synthetic, or recombinantly produced binding partner for a biological molecule or another target of interest. Exemplary binding domains of this disclosure include, for example, a Fab′, F(ab′)₂, Fab, Fv, rIgG, scFv, hcAbs (heavy chain antibodies), a single domain antibody, V_{HH}, V_{NAR}, sdAbs, nanobody, receptor ectodomains or ligand-binding portions thereof, or ligands (e.g., cytokines, chemokines). A “Fab” (fragment antigen binding) is the part of an antibody that binds to antigens and includes the variable region and CH1 of the heavy chain linked to the light chain via an inter-chain disulfide bond. A variety of assays are known for identifying binding domains of the present disclosure that specifically bind a particular target, including Western blot, ELISA, and Biacore® analysis. Particularly preferred binding domains comprise immunoglobulin light and heavy chain variable domains (e.g., scFv, Fab) and are herein referred to as “immunoglobulin binding domains” or “immunoglobulin binding proteins.” Immunoglobulin binding domains can be incorporated into a variety of protein scaffolds or structures as described herein, such as an antibody or an antigen binding fragment thereof, a scFv-Fc fusion protein, or a fusion protein comprising two or more of such immunoglobulin binding domains.

Throughout this disclosure, the term “antibody” refers to an immunoglobulin molecule that specifically binds to, or is immunologically reactive toward, an antigen. The portion of the antibody that binds the antigen may be referred to as an “antigen binding domain.” In certain embodiments, an antibody is an intact antibody comprising at least two heavy (H) chains and two light (L) chains inter-connected by disulfide bonds, as well as an antigen-binding portion of an intact antibody that has or retains the capacity to bind a target molecule. An antibody or an antigen binding fragment thereof of this disclosure can include, for example, polyclonal, monoclonal, and genetically engineered antibodies. A monoclonal antibody or antigen-binding portion thereof of this disclosure can be, for example, non-human (e.g., murine, rabbit), chimeric, humanized, or human. In certain embodiments, an antibody of this disclosure is a heteroconjugate, bispecific, multi-specific, diabody, triabody, or tetrabody. Immunoglobulin structure and function are reviewed, for example, in Greenfield, Ed., Antibodies: A Laboratory Manual, Chapters 2 and 3 (Cold Spring Harbor Laboratory, Cold Spring Harbor, 2014).

For example, the terms “V_{L}” and “V_{H}” refer to the variable binding region from an antibody light and heavy chain, respectively. The variable binding regions are made up of discrete, well-defined sub-regions known as “complementarity determining regions” (CDRs) and “framework regions” (FRs). The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM).

A binding domain and a fusion protein thereof “specifically binds” a target if it binds the target with an affinity or Kₐ (i.e., an equilibrium association constant of a particular binding interaction with units of 1/M) equal to or greater than 10⁵ M⁻¹, while not significantly binding other components present in a test sample. Binding domains (or fusion proteins thereof) may be classified as “high affinity” binding domains (or fusion proteins thereof) and “low affinity” binding domains (or fusion proteins thereof). “High affinity” binding domains refer to those binding domains with a Kₐ of at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹, preferably at least 10⁸ M⁻¹ or at least 10⁹ M⁻¹. “Low affinity” binding domains refer to those binding domains with a Kₐ of up to 10⁸ M⁻¹, up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M). Affinities of binding domain polypeptides and fusion proteins according to the present disclosure can be readily determined using conventional techniques (see, e.g., Scatchard et al., Ann. N.Y. Acad. Sci. 51:660, 1949; and U.S. Pat. Nos. 5,283,173, 5,468,614, or the equivalent).

“Derivative,” as used herein, refers to a chemically or biologically modified version of a compound that is structurally similar to a parent compound and (actually or theoretically) derivable from that parent compound. Generally, a “derivative” differs from an “analogue” in that a parent compound may be the starting material to generate a “derivative,” whereas the parent compound may not necessarily be used as the starting material to generate an “analogue.” An analogue may have different chemical or physical properties of the parent compound. For example, a derivative may be more hydrophilic or it may have altered reactivity (e.g., a CDR having an amino acid change that alters its affinity for a target) as compared to the parent compound.

As used herein, “identical” or “identity” refer to the similarity between a DNA, RNA, nucleotide, amino acid, or protein sequence to another DNA, RNA, nucleotide, amino acid, or protein sequence. Identity can be expressed in terms of a percentage of sequence identity of a first sequence to a second sequence. Percent (%) sequence identity with respect to a reference DNA sequence can be the percentage of DNA nucleotides in a candidate sequence that are identical with the DNA nucleotides in the reference DNA sequence after aligning the sequences. Percent (%) sequence identity with respect to a reference amino acid sequence can be the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity. For example, the percent sequence identity values for sequences provided herein can be generated using the NCBI BLAST 2.0 software as defined by Altschul et al., “Gapped BLAST and PSI-BLAST: a new generation of protein database search programs,” Nucleic Acids Res. 2007, 25, 3389-3402, with the parameters set to default values.

“Oxo” refers to a radical with the formula ═O.

“Nitro” refers to a radical with the formula —NO₂.

“Thioxo” refers to a radical with the formula ═S.

“Cyano” refers to a radical with the formula —CN.

“Amino” refers to a radical with the formula —NH₂.

“Hydroxyl” or “hydroxy” refers to a radical with the formula —OH.

“Thiol” refers to a radical with the formula —SH.

“Aldehyde” refers to a radical with the formula —C(═O)H.

“Carboxyl” refers to a functional group of the formula —C(═O)OH.

“Halo” refers to a halogen radical—e.g., —F, —Cl, —Br, —I, etc.

“Alkyl” refers to a saturated, straight or branched hydrocarbon chain radical consisting solely of carbon and hydrogen atoms, having from one to twelve carbon atoms (C₁-C₁₂ alkyl), one to eight carbon atoms (C₁-C₈ alkyl) or one to six carbon atoms (C₁-C₆ alkyl), or any value within these ranges, such as C₄-C₆ alkyl or the like, and which is attached to the rest of the molecule by a single bond, e.g., methyl, ethyl, npropyl, 1methylethyl (isopropyl), nbutyl, npentyl, 1,1dimethylethyl (tbutyl), 3methylhexyl, 2methylhexyl or the like. The number of carbons referred to relates to the carbon backbone and carbon branching but does not include carbon atoms belonging to any substituents. Similarly, an alkenyl refers to a straight or branched hydrocarbon chain radical consisting of carbon and hydrogen having at least one carbon-carbon double bond. An “alkynyl” contains at least one carbon-carbon triple bond. Unless stated otherwise specifically in the specification, an alkyl, an alkenyl, or an alkynl group is optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, or the like. Unless stated otherwise specifically in the specification, a haloalkyl group is optionally substituted.

“Alkoxy” refers to a radical of the formula —ORₐ where Rₐ is an alkyl radical as defined above containing one to twelve carbon atoms (C₁-C₁₂ alkoxy), one to eight carbon atoms (C₁-C₈ alkoxy) or one to six carbon atoms (C₁-C₆ alkoxy), or any value within these ranges. Unless stated otherwise specifically in the specification, an alkoxy group is optionally substituted.

“Haloalkoxy” refers to an alkoxy radical, as defined above that is substituted by one or more halo radicals, as defined above. Unless stated otherwise specifically in the specification, a haloalkoxy group is optionally substituted.

“Aminyl” refers to a radical of the formula —NRₐR_{b}, where Rₐ and R_{b} are each independently H or C₁-C₆ alkyl as defined above. When both of Rₐ and R_{b} are H, an “aminyl” group is the same as an “amino” group as defined above. The C₁-C₆ alkyl portion of an aminyl group is optionally substituted unless stated otherwise.

“Amindyl” refers to a radical of the formula —C(═O)NRₐR_{b} where Rₐ and R_{b} are each independently H or C₁-C₆ alkyl as defined above. Alternatively, aminyl may also refer to the radical or —N(Rₐ)C(═O)R_{b} where Rₐ is either H or C₁-C₆ alkyl and R_{b} is C₁-C₆ alkyl. The C₁-C₆ alkyl portion of an aminyl group is optionally substituted unless stated otherwise.

“Carbohydrate” refers to a radical consisting of carbon, hydrogen, and oxygen atoms. In some embodiments, a carbohydrate has a hydrogen to oxygen ratio of 2:1. In some embodiments, the carbohydrate has an empirical formula of Cₘ(H₂O)ₙ where m and n are integers that may or may not be the same. Exemplary carbohydrates include sugars (e.g., monosaccharides, disaccharides, oligosaccharides, polysaccharides), starch, and cellulose. In some embodiments, the carbohydrate is selected from the group consisting of glucose, fructose, sucrose, ribose, amylose, lactose, galactose, xylose, maltose, isomaltulose, trehalose, sorbitol, mannitol, maltodextrin, raffinose, stachyose, amylose, amylopectin, glycogen, cellulose, hemicellulose, pectins, hydrocolloids, or the like.

“Haloalkyl” refers to an alkyl radical, as defined above that is substituted by one or more halo radicals. The haloalkyl radical is joined at the main chain through the alkyl carbon atom. Unless stated otherwise specifically in the specification, a haloalkyl group is optionally substituted.

“Carboxyalkyl” refers to an alkyl radical, as defined above that is substituted by one or more carboxy radicals. The carboxyalkyl radical is joined at the main chain through the alkyl carbon atom. Unless stated otherwise specifically in the specification, a carboxyalkyl group is optionally substituted.

“Cycloalkyl” or “carbocyclic ring” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, or the like. Unless otherwise stated specifically in the specification, a cycloalkyl group may be optionally substituted.

“Aryl” refers to any stable monocyclic, bicyclic, or polycyclic carbon ring system of from 4 to 12 atoms in each ring, wherein at least one ring is aromatic. Some examples of an aryl include phenyl (sometimes denoted as “Ph”), naphthyl, tetrahydro-naphthyl, indanyl, anthracyl, and biphenyl. Where an aryl substituent is bicyclic and one ring is non-aromatic, it is understood that attachment is to the aromatic ring. An aryl may be substituted or unsubstituted.

The term “heterocycle” or “heterocyclyl” refers to an aromatic or nonaromatic ring system of from five to twenty-two atoms, wherein from 1 to 4 of the ring atoms are heteroatoms selected from oxygen, nitrogen, and sulfur. Thus, a heterocycle may be a heteroaryl or a dihydro or tetrathydro version thereof. Heterocycles include pyrrolidine, tetryhydrofuran, thiolane, indolinyl, 3H-indolyl, azetidine, oxetane, thietane, diazetidine, dioxetane, dithietane, piperidine, tetrahydrofuran, pyran, tetrahydropyran, thiacyclohexane, tetrahydrothiophene, pyridine, pyrimidine, or the like.

“Heteroaryl” refers to any stable monocyclic, bicyclic, or polycyclic carbon ring system of from 4 to 12 atoms in each ring, wherein at least one ring is aromatic and contains from 1 to 4 heteroatoms selected from oxygen, nitrogen and sulfur. Some examples of a heteroaryl include acridinyl, quinoxalinyl, pyrazolyl, indolyl, benzotriazolyl, furanyl, thienyl, benzothienyl, benzofuranyl, quinolinyl, isoquinolinyl, oxazolyl, isoxazolyl, pyrazinyl, pyridazinyl, pyridinyl, pyrimidinyl, pyrrolyl, and tetrahydroquinolinyl. A heteroaryl includes the N-oxide derivative of a nitrogen-containing heteroaryl.

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, e.g., methylene, ethylene, propylene, n-butylene, or the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, alkylene is optionally substituted.

Similarly, “alkenylene” refers to a straight or branched divalent hydrocarbon chain linking the rest of the molecule to a radical group, consisting solely of carbon and hydrogen and containing at least one double carbon-carbon bond. An alkenylene chain may contain between two and twelve carbon atoms. An “alkynylene” is also a straight or branched divalent hydrocarbon chain linking the rest of the molecule to a radical group, consisting solely of carbon and hydrogen, but contains at least one triple carbon-carbon bond.

“Heteroalkylene” refers to an alkylene group, as defined above, comprising at least one heteroatom (e.g., Si, N, O, P or S) within the alkylene chain or at a terminus of the alkylene chain. In some embodiments, a heteroatom is within the alkylene chain (i.e., a heteroalkylene comprises at least one carbon-[heteroatom]ₓ-carbon bond, where x is 1, 2 or 3). In other embodiments, a heteroatom is at a terminus of the alkylene and thus serves to join the alkylene to the remainder of the molecule (e.g., M1-H-A-M2, where M1 and M2 are portions of the molecule, H is a heteroatom and A is an alkylene). Unless stated otherwise specifically in the specification, a heteroalkylene group is optionally substituted. Exemplary heteroalkylene groups include ethylene oxide (e.g., polyethylene oxide), propylene oxide, amino acid chains (i.e., short to medium length peptides—containing 1-15 amino acids), and alkylene chains connected via a variety of functional groups such as amides, disulfides, phosphates, sulfates, sulfonamides, esters, ethers, —S—, carbamates, ureas, thioureas, anhydrides, or the like (including combinations thereof). In some embodiments, a heteroalkylene includes a polyamino acid having 1-10 amino acids. In some embodiments, a heteroalkylene includes a polyamino acid having 1-5 amino acids.

“Heteroalkenylene” refers to a heteroalkylene group, as defined above, that contains at least one carbon-carbon double bond. “Heteroalkynylene” refers to a heteroalkylene group, as defined above, that contains at least one carbon-carbon triple bond.

“Heteroatomic linker” refers to a contiguous chain of heteroatoms or a heteroatom that connects a portion of the molecule to a radical group. A heteroatomic linker may be multivalent (e.g., divalent, trivalent, etc.). A heteroatomic linker consists solely of non-carbon atoms (e.g., H, O, N, S, Si, P, etc.). Unless otherwise stated specifically in the specification, a heteroatomic linker may be optionally substituted.

“Cycloalkylene” is a multivalent (e.g., divalent, trivalent, etc.) cycloalkyl group. Unless otherwise stated specifically in the specification, a cycloalkylene group may be optionally substituted.

“Arylene” is a multivalent (e.g., divalent, trivalent, etc.) aryl group. Unless otherwise stated specifically in the specification, an arylene group may be optionally substituted.

“Heterocyclylene” is a multivalent (e.g., divalent, trivalent, etc.) heterocyclyl group. Unless otherwise stated specifically in the specification, a heterocyclylene group may be optionally substituted.

“Heteroarylene” is a multivalent (e.g., divalent, trivalent, etc.) heteroaryl group. Unless otherwise stated specifically in the specification, a heteroarylene group may be optionally substituted.

A “linker” refers to a contiguous chain of at least one atom, such as carbon, oxygen, silicon, nitrogen, sulfur, phosphorous, and combinations thereof, which connects a portion of a molecule to another portion of the same molecule or to a different molecule or fragment thereof via a covalent bond (e.g., a single bond, double bond, or triple bond). In some embodiments, the linker is optionally substituted alkylene linker, an optionally substituted alkenylene linker, an optionally substituted alkynylene linker, an optionally substituted heteroalkylene linker, an optionally substituted heteroalkenylene linker, an optionally substituted heteroalkynylene linker, a heteroatomic linker, a cycloalkylene linker, an arylene linker, a heterocyclylene linker, a heteroarylylene linker, or combinations thereof. Unless otherwise stated specifically in the specification, a linker may be optionally substituted.

“Trigger element” refers to a molecular motif that is recognized by a biological molecule (e.g., a protease such as cathepsin) or susceptible to a chemical reaction in a biological environment (e.g., acid labile). Typically an enzymatic recognition of the trigger element results in a catalyzed cleavage reaction that results in release of a payload from the antibody+linker(s).

“Immolative element” or “self-immolative spacer” refers to chemically labile group that facilitates release or cleavage between portions of a molecule (e.g., between an antibody-linker and a payload). An immolative element typically a divalent linker motif that is amenable to one or more chemical reactions that ultimately results in cleavage of the covalent bonds between the two terminal ends of the group. The reactions occur as a result some type of stimuli to the system (e.g., a protease catalyzing the reaction during its recognition of a trigger element or decrease in pH when the antibody-drug conjugate is subjected to an intracellular environment) which then results in controlled release of a payload. Commonly used immolative elements are the para-aminobenzyloxycarbonyl (PABC) and aminal. In some embodiments, an immolative element has one of the following structures:

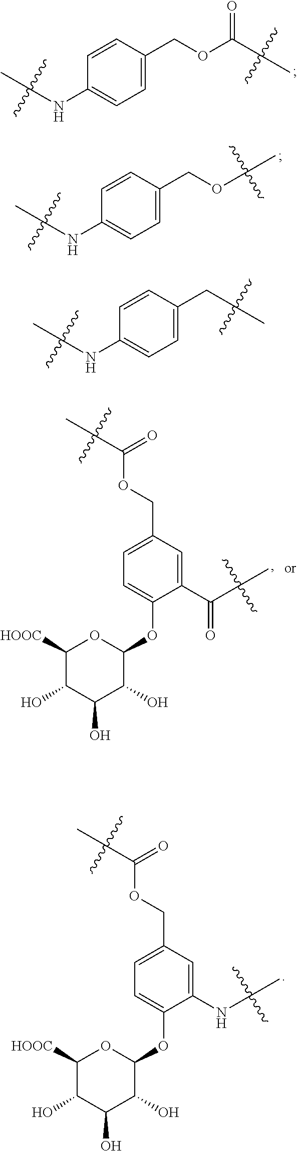 [SMILES: *Nc1ccc(COC(*)=O)cc1 ; *Nc1ccc(CO*)cc1 ; *Cc1ccc(N*)cc1 ; *C(=O)OCc1ccc(O[C@@H]2O[C@H](C(=O)O)[C@@H](O)[C@H](O)[C@H]2O)c(C(*)=O)c1 ; *Nc1cc(COC(*)=O)ccc1O[C@@H]1O[C@H](C(=O)O)[C@@H](O)[C@H](O)[C@H]1O]

The following scheme depicts the fragmentation of para-aminobenzyloxycarbonyl (PABC) and release of a payload:

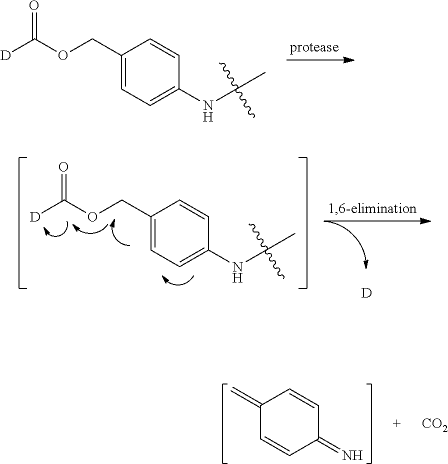

wherein D represents the unmodified payload.

In some embodiments, an immolative element has the following structure:

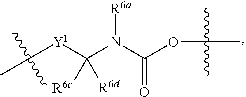

wherein:
R^{6a}, R^{6b}, R^{6c}, and R^{6d} are independently hydrogen, an optionally substituted alkyl, an optionally substituted aryl, or optionally substituted heteroaryl, or
R^{6a} and R^{6c} together with the nitrogen and carbon atoms to which they are attached form azetidinyl, pyrrolodinyl, piperidinyl, or homopiperidinyl and R^{6d} is hydrogen; and
Y¹ is —O—, —S—, or —NR^{6b}—.

In some embodiments, an immolative element has the following structure:

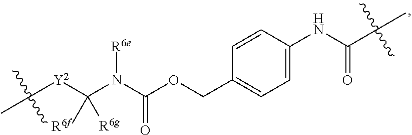

wherein:
R^{6e}, R^{6f}, R^{6g}, and R^{6h} are independently hydrogen, an optionally substituted alkyl, an optionally substituted aryl, or optionally substituted heteroaryl, or
R^{6a} and R^{6c} together with the nitrogen and carbon atoms to which they are attached form azetidinyl, pyrrolodinyl, piperidinyl, or homopiperidinyl and R^{6d} is hydrogen; and
Y² is —O—, —S—, or —NR^{6f}—.

In certain embodiments, an immolative element has the following structure:

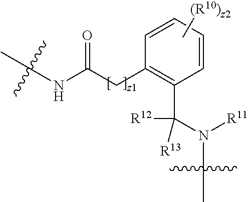

wherein:
each occurrence of R¹⁰ is independently alkyl, alkoxy, or halo;
R¹¹ is hydrogen, alkyl, or —(CH₂CH₂O)_{z3}—CH₃;
R¹² is hydrogen or alkyl;
R¹³ is hydrogen or alkyl;
z1 is 0 or 1;
z2 is 0, 1, 2, 3, or 4; and
z3 is 0, 1, 2, 3, 4, 5, 6, 7, 8, 9, or 10.

In some embodiments, an immolative element has one of the following structures:

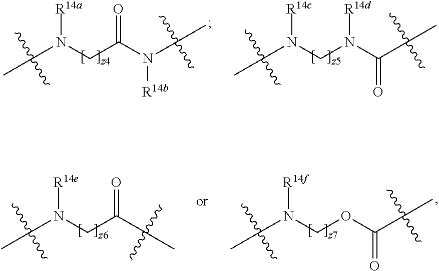

wherein:
R^{14a}, R^{14b}, R^{14c}, R^{14d}, R^{14e}, and R^{14f} are each independently hydrogen, alkyl, hydroxyalkyl, or alkoxyalkyl;
z4, z5, z6, and z7 are each independently 1, 2, 3, 4, 5, or 6.

The foregoing terms include all systems that comprise effective cleavage elements in the endo-lysosomal processing of antibody-drug conjugates. Exemplary systems include cathepsin-based enzymatic peptide sequences, such as Val-Cit, Val-Ala, Ala-Ala, Gly-Gly-Phe-Gly, or the like. Also included are trigger elements that are sensitive to the enzyme b-glucuronidase, which contain a glucuronic acid as a trigger element. In some embodiments, a trigger element is directly and covalently bound to an immolative element. In some embodiments, an immolative element is directly and covalently bound to a payload and once cleavage occurs, a payload is then irreversibly released.

“Non-cleavable element” refers to a linker that does not include either a trigger element or an immolative element and is not cleaved under normal physiological conditions. Depending on the target, many conjugates can be quite effective without a formal cleavage release element. Accordingly, in some embodiments a payload may be directly attached to a phenyl maleimide via a non-cleavable linker.

“Heteroalkylene element” refers to a linker comprising one or more heteroalkylene, as defined above. In some embodiments, a heteroalkylene element can be used to optimize characteristics of the linker-payload. In some embodiments, a heteroalkylene element(s) increases a linear connection between other elements (e.g., charged elements, hydrophilic elements, etc.). In some embodiments, a heteroalkylene element increases the distance between a polar cap or a payload of the phenyl-maleimide portion of Structure (I). Heteroalkylene elements vary in length, structure, polarity, degree of branching, or the like. The aforementioned variables allow the linker-payload and, in turn, the antibody-linker-drug conjugate to have the best overall properties (e.g., solubility, to be monodisperse, etc.).

In some embodiments, a heteroalkylene element comprises a linear polyethylene glycol with two ethylene glycol units (i.e., PEG₂) and one or more amino acid(s). In another embodiment, a heteroalkylene element comprises a Gly-Gly dipeptide. In some embodiments, a heteroalkylene element includes a PEG₁₋₂₄, di-, tri- and tetra-peptide, or combinations thereof. In other embodiments, a heteroalkylene element comprises amino acids. In another embodiment, a heteroalkylene element comprises PEG₁₋₂₄. In another embodiment, a heteroalkylene element comprises PEG₁₋₁₂. In another embodiment, a heteroalkylene element comprises PEG₂₋₆. In some embodiments, both PEGₓ and amino acids combine to form a heteroalkylene element. Exemplary linear PEG moieties can be found, e.g., in PCT Publication No. WO 2021/207701, which PEG moieties are hereby incorporated by reference in its entirety.

“Hydrophilic element” refers to a portion of Structure (I) that effectively promotes solubility in aqueous solvents (e.g., water, phosphate buffered saline) for the entirety of the molecule. In effect, a hydrophilic element conceals or counteracts hydrophobic portions of the linker-payload such that the resultant molecule is stable in an aqueous environment (e.g., pH 7.4 buffered water). In some embodiments, a hydrophilic element produces a stable and soluble linker-payload with improved overall physicochemical properties. A hydrophilic element provides a suitable chemical functionality that enables stable conjugation to a protein. In some embodiments, incorporation of an appropriate hydrophilic element(s) leads to the resultant protein conjugate to possess an ADME/DMPK profile that is more protein-like.

In some embodiments, a hydrophilic element includes PEG linkers of medium length (10-14 units or PEG₁₀₋₁₄). In some embodiments, a hydrophilic element includes PEG lengths from 2-24 units (i.e., PEG₂₋₂₄). In some embodiments, the length and amount of branching included in a hydrophilic element can be adjusted based on other elements present in Structure (I). In some embodiments, the hydrophilic element is a C₁-C₆ alkoxy (e.g., methoxy). In some embodiments, a hydrophilic element comprises poly-sarcosine (PSAR). One aspect a hydrophilic element imparts polarity and hydrophilicity to the overall conjugate. In some embodiments, a hydrophilic element is large enough in size and flexible enough in structure to mask hydrophobicity with or without a trigger element. In some embodiments, a hydrophilic element is divalent. In some embodiments, a hydrophilic element is a monovalent radical. In some embodiments, a hydrophilic element is multivalent. In some embodiments, a hydrophilic element is branched. In some embodiments, a hydrophilic element is linear.

Hydrophilic groups (e.g., moieties that make up a hydrophilic element) are known to those of ordinary skill in the art. For example, hydrophilic groups can be found in PCT Publication Nos. WO 2019/217591, WO 2018/089373 and the hydrophilic groups from each are hereby incorporated by reference in their entirety.

“Polar cap” refers to a radical having a structure that includes one or more polar or hydrophilic functional groups (e.g., one or more —OH, —NH₂, —C(═O)OH, —S(O)₃, —OP(O)₃, or the like). In some embodiments, a polar cap is attached to a terminus of Structure (I). In some embodiments, a polar cap is attached to more than one termini of Structure (I) (e.g., if a polar cap is attached to multiple termini of a branched heteroalkylene element or a branched hydrophilic element). A polar cap imparts hydrophilicity unto the overall compound of Structure (I). In some embodiments, a polar cap comprises one or more moiety that is negatively charged under physiological conditions (e.g., a phosphate, carboxylic acid, sulfate, sulfonic acid, etc.). In some embodiments, a polar cap comprises one or more moiety that is positively charged under physiological conditions (e.g., a quaternary amine). In some embodiments, a polar cap itself is zwitterionic under physiological conditions with an overall negative, an overall positive, or an overall neutral charge. In some embodiments, a polar cap serves to increase both polarity and charge by the incorporation of an amino acid, which may have additional functionality to add further polarity and charge. In some embodiments, a polar cap includes L-Glutamic acid. In some embodiments, a polar cap has two carboxy groups. In some embodiments, a polar cap includes one or more di-acids. In some embodiments, the glutamic acid is further modified to have one or both acids amidated with poly-ol-containing amines. In some embodiments, a polar cap comprises amino acids that have a glycoside moiety (e.g., L-serine-beta-D-glucoside or the like).

“Stable compound” and “stable structure” are meant to indicate a compound that is sufficiently robust to survive isolation to a useful degree of purity from a reaction mixture, and formulation into an efficacious therapeutic agent.

“Salt” includes both acid (e.g., salts formed with inorganic or organic acids such as hydrochloric acid, hydrobromic acid, sulfuric acid, acetic acid, ascorbic acid, etc.) and base (e.g., salts formed with inorganic or organic bases such as sodium, potassium, amine bases, etc.) addition salts.

Crystallizations may produce a solvate of the compounds described herein (e.g., a compound of Structure (I)). Embodiments of the present disclosure include all solvates of the described compounds. As used herein, the term “solvate” refers to an aggregate that comprises one or more molecules of a compound of this disclosure with one or more molecules of solvent.

Embodiments of the compounds of this disclosure (e.g., compounds of Structure (I)), or their salts, tautomers, or solvates may contain one or more stereocenters and may thus give rise to enantiomers, diastereomers, and other stereoisomeric forms that may be defined, in terms of absolute stereochemistry, as (R)- or (S)- or, as (D)- or (L)- for amino acids. Embodiments of the present disclosure are meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other features giving rise to geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are non-superimposable mirror images of one another.

A “tautomer” refers to a proton shift from one atom of a molecule to another atom of the same molecule. The present disclosure includes tautomers of any said compounds. Various tautomeric forms of the compounds are easily derivable by those of ordinary skill in the art.

The chemical naming protocol and structure diagrams used herein are a modified form of the I.U.P.A.C. nomenclature system, using the ACD/Name Version 9.07 software program and/or ChemDraw Ultra Version 11.0 software naming program (CambridgeSoft). Common names familiar to one of ordinary skill in the art are also used.

For ease of illustration, various compounds of Structure (I) or conjugates of Structure (II) with sulfur or phosphorus containing moieties (e.g., sulfonate, phosphate, or the like) may be described in an anionic state (e.g., —S(O)₃ or —OP(O)₃). One of skill in the art will readily understand that the charge is dependent on pH and the uncharged (e.g., protonated form(s) or salt form(s), such as sodium or other cation) forms are also included in the scope of embodiments of this disclosure.

### Linker Compounds

In certain embodiments, the present disclosure provides branched phenyl maleimide compounds (e.g., compounds of Structure (I)) enable the formation of a covalent bond between a linker-payload and a protein. As noted above, in certain embodiments of the present disclosure, compounds useful as linkers between payloads (including fragments thereof) and targeting peptides (including fragments thereof—e.g., antibodies) are provided.

Accordingly, some embodiments provide a compound having the following Structure (I):

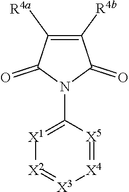

wherein:
one of X¹, X², X³, X⁴ and X⁵ is C-L¹-R¹, another one of X¹, X², X³, X⁴ and X⁵ is C-L²-R², and the remaining three of X¹, X², X³, X⁴ and X⁵ are each independently N, C—R³, or C-L³-R^{3a};
R¹, R², and R^{3a} each independently comprise one or more moieties selected from an amino acid element, a charged element, a heteroalkylene element, a hydrophilic element, a trigger element, an immolative element, a polar cap, a payload, and combinations thereof; provided that at least one of R¹ and R² comprises a payload;
each occurrence of R³ is independently selected from the group consisting of hydrogen, deuterium, alkyl, haloalkyl, halo, alkoxy, haloalkoxy, amino, aminyl, amidyl, aldehyde, hydroxyl, cyano, nitro, thiol, carboxy, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, —P(O)₃H, alkyl-O—P(O)₃-alkyl, alkyl-P(O)₃-alkyl, —O-alkyl-S(O)₃-alkyl, —O-alkyl-O—P(O)₃-alkyl, —O-alkyl-P(O)₃-alkyl, —S(O)₃-alkyl, —OP(O)₃-alkyl, —P(O)₃-alkyl, sulfamide, sulfinimide, and a carbohydrate;
R^{4a} and R^{4b} are each independently hydrogen, deuterium, halo, or —S—R^{4c} wherein R^{4c} is substituted or unsubstituted C₆-C₁₀ aryl or substituted or unsubstituted 5-12 membered heteroaryl; and
L¹, L², and L³ are each independently a linker comprising an optionally substituted alkylene, an optionally substituted alkenylene, an optionally substituted alkynylene, an optionally substituted heteroalkylene, an optionally substituted heteroalkenylene, an optionally substituted heteroalkynylene, a heteroatomic linker, an optionally substituted cycloalkylene, an optionally substituted arylene, an optionally substituted heterocyclylene, an optionally substituted heteroarylene, or combinations thereof;
   as a stereoisomer, enantiomer or tautomer thereof or a mixture thereof; or a pharmaceutically acceptable salt, solvate or prodrug thereof.

Certain embodiments provide a compound having the following Structure (I):

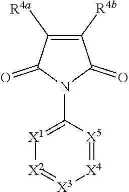

wherein:
one of X¹, X², X³, X⁴ and X⁵ is C-L¹-R¹, another one of X¹, X², X³, X⁴ and X⁵ is C-L²-R², and the remaining three of X¹, X², X³, X⁴ and X⁵ are each independently N, C—R³, or C-L³-R^{3a};
R¹, R², and R^{3a} each independently comprise one or more moieties selected from an amino acid element, a charged element, a heteroalkylene element, a hydrophilic element, a trigger element, an immolative element, a polar cap, a payload, and combinations thereof; provided that at least one of R¹ and R² comprises a payload;
each occurrence of R³ is independently selected from the group consisting of hydrogen, deuterium, alkyl, haloalkyl, halo, alkoxy, haloalkoxy, amino, aminyl, amidyl, aldehyde, hydroxyl, cyano, nitro, thiol, carboxy, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, —P(O)₃H, alkyl-O—P(O)₃-alkyl, alkyl-P(O)₃-alkyl, —O-alkyl-S(O)₃-alkyl, —O-alkyl-O—P(O)₃-alkyl, —O-alkyl-P(O)₃-alkyl, —S(O)₃-alkyl, —OP(O)₃-alkyl, —P(O)₃-alkyl, sulfamide, sulfinimide, and a carbohydrate;
R^{4a} and R^{4b} are each independently hydrogen, deuterium, halo, or —S—R^{4c} wherein R^{4c} is substituted or unsubstituted C₆-C₁₀ aryl or substituted or unsubstituted 5-12 membered heteroaryl; and
L¹, L², and L³ are each independently direct bond or a linker comprising an optionally substituted alkylene, an optionally substituted alkenylene, an optionally substituted alkynylene, an optionally substituted heteroalkylene, an optionally substituted heteroalkenylene, an optionally substituted heteroalkynylene, a heteroatomic linker, an optionally substituted cycloalkylene, an optionally substituted arylene, an optionally substituted heterocyclylene, an optionally substituted heteroarylene, or combinations thereof;
   as a stereoisomer, enantiomer or tautomer thereof or a mixture thereof; or a pharmaceutically acceptable salt, solvate or prodrug thereof.

In some embodiments, R^{4a} and R^{4b} are both hydrogen. In some embodiments, R^{4a} is halo or R^{4b} is halo. In some embodiments, R^{4a}, R^{4b}, or both have one of the following structures:

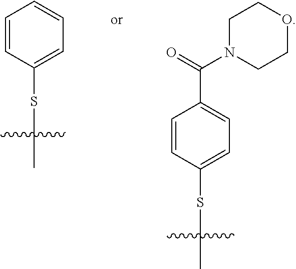 [SMILES: *Sc1ccccc1 ; *Sc1ccc(C(=O)N2CCOCC2)cc1]

In some embodiments, R¹ R², and/or R^{3a} comprises elements selected from an amino acid element, a charged element, a heteroalkylene element, a hydrophilic element, a trigger element, an immolative element, a polar cap, a payload, and combinations thereof. It is understood that these elements can be connected in any order and be connected in a linear manner or via a branched connection. In some embodiments, R¹ R², and/or R^{3a} comprises multiple occurrences of an element (e.g., two or more heteroalkylene elements, two or more hydrophilic elements, two or more polar caps, etc.).

In some embodiments, R¹, R², or R^{3a} comprises a branch point as part of an amino acid element (e.g., lysine) wherein additional elements are attached via an epsilon amine of the lysine and other additional elements are linked to the amino acid element via one or more peptide bonds to the alpha carbon of a lysine. A similar motif could be utilized with a glutamic acid of an amino acid element. In some embodiments, an amino acid element comprises one of the following structures:

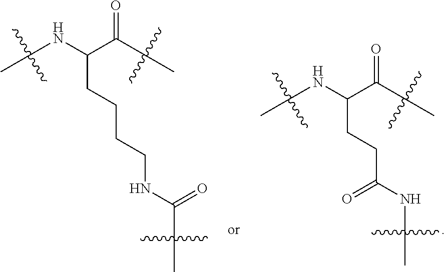 [SMILES: *NC(CCCCNC(*)=O)C(*)=O ; *NC(=O)CCC(N*)C(*)=O]

In some embodiments, a compound of Structure (I) comprises one of the following structures:

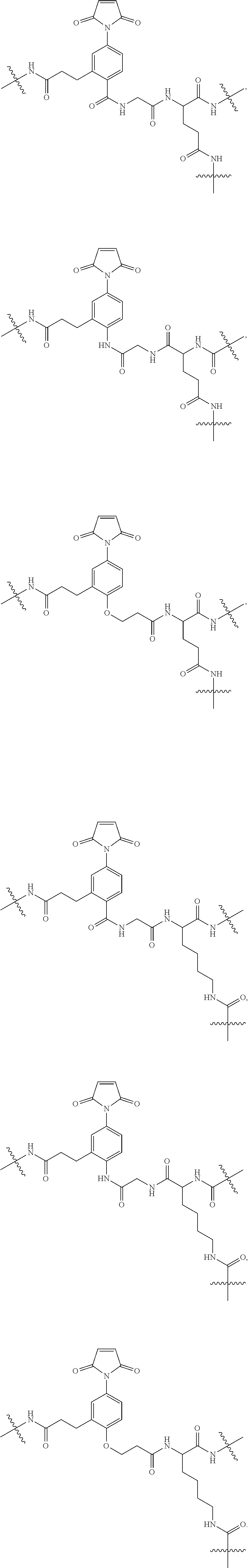 [SMILES: *NC(=O)CCc1cc(N2C(=O)C=CC2=O)ccc1C(=O)NCC(=O)NC(CCC(=O)N*)C(=O)N* ; *NC(=O)CCc1cc(N2C(=O)C=CC2=O)ccc1NC(=O)CNC(=O)C(CCC(=O)N*)NC(*)=O ; *NC(=O)CCc1cc(N2C(=O)C=CC2=O)ccc1OCCC(=O)NC(CCC(=O)N*)C(=O)N* ; *NC(=O)CCc1cc(N2C(=O)C=CC2=O)ccc1C(=O)NCC(=O)NC(CCCCNC(*)=O)C(=O)N* ; *NC(=O)CCc1cc(N2C(=O)C=CC2=O)ccc1NC(=O)CNC(=O)C(CCCCNC(*)=O)NC(*)=O ; *NC(=O)CCc1cc(N2C(=O)C=CC2=O)ccc1OCCC(=O)NC(CCCCNC(*)=O)C(=O)N*]

In certain embodiments, R¹ has the following structure:

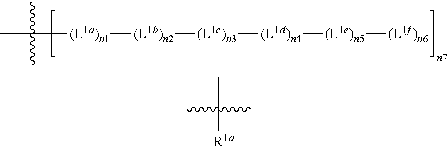

wherein:
L^{1a} is an amino acid element;
L^{1b} is a charged element;
L^{1c} is a heteroalkylene element;
L^{1d} is a hydrophilic element;
L^{1e} is a trigger element; and
L^{1f} is an immolative element;
   wherein one or more occurrence of L^{1a}, L^{1b}, L^{1c}, L^{1d}, L^{1e}, and L^{1f} optionally joins with one or more of another of L^{1a}, L^{1b}, L^{1c}, L^{1d}, L^{1e}, and L^{1f} to form one or more ring;
each occurrence of n1, n2, n3, n4, n5, and n6 is independently an integer from 0-3, provided that n1+n2+n3+n4+n5+n6=1 or more;
n7 is 1, 2, 3, 4, 5, or 6; and
R^{1a} is a payload that is covalently bound to one occurrence of L^{1a}, L^{1b}, L^{1c}, L^{1d}, L^{1e}, or L^{1f} and the payload is optionally substituted with a polar cap.

In some embodiments, n7 is 1, 2, or 3. In some embodiments, n7 is 1 or 2. In some embodiments, n7 is 1.

In some embodiments, R¹ has the following structure:

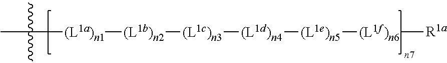

wherein:
L^{1a} is an amino acid element;
L^{1b} is a charged element;
L^{1c} is a heteroalkylene element;
L^{1d} is a hydrophilic element;
L^{1e} is a trigger element; and
L^{1f} is an immolative element;
   wherein one or more occurrence of L^{1a}, L^{1b}, L^{1c}, L^{1d}, L^{1e}, and L^{1f} optionally joins with one or more of another of L^{1a}, L^{1b}, L^{1c}, L^{1d}, L^{1e}, and L^{1f} to form one or more ring;
each occurrence of n1, n2, n3, n4, n5, and n6 is independently an integer from 0-3, provided that n1+n2+n3+n4+n5+n6=1 or more;
n7 is 1, 2, 3, 4, 5, or 6; and
R^{1a} is a payload optionally substituted with a polar cap.

In more embodiments, R² has the following structure:

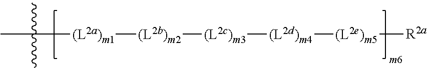

wherein:
L^{2a} is an amino acid element;
L^{2b} is a charged element;
L^{2c} is a heteroalkylene element;
L^{2d} is a hydrophilic element;
L^{2e} is a trigger element;
each occurrence of m1, m2, m3, m4, and m5 is independently an integer from 0-3, provided that m1+m2+m3+m4+m5=1 or more;
m6 is 1, 2, 3, 4, or 5; and
R^{2a} is hydrogen, alkyl, a payload, or a polar cap.

In some embodiments, m6 is 1, 2, or 3. In some embodiments, m6 is 1 or 2. In some embodiments, m6 is 1.

In still more embodiments, R^{3a} has the following structure:

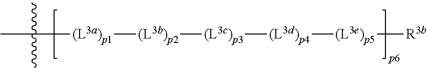

wherein:
L^{3a} is an amino acid element;
L^{3b} is a charged element;
L^{3c} is a heteroalkylene element;
L^{3d} is a hydrophilic element;
L^{3e} is a trigger element;
each occurrence of p1, p2, p3, p4, and p5 is independently an integer from 0-3, provided that p1+p2+p3+p4+p5=1 or more;
p6 is 1, 2, 3, 4, or 5; and
R^{3b} is hydrogen, alkyl, or a polar cap.

In some embodiments, p6 is 1, 2, or 3. In some embodiments, p6 is 1 or 2. In some embodiments, p6 is 1.

In certain embodiments, n7 is 1 and each of n1 through n6 are 1. In some embodiments, n7 is 1 and each of n1 through n4 are 0, n5 is 1, and n6 is 1. In certain embodiments, n7 is 1 and n1 is 0, n2 is 0, n3 is 1, n4 is 0, n5 is 1, and n6 is 1. In certain embodiments, n7 is 1 and n1 is 1, n2 is 0, n3 is 1, n4 is 0, n5 is 1, and n6 is 1. In certain embodiments, n7 is 1 and n1 is 1, n2 is 1, n3 is 1, n4 is 0, n5 is 1, and n6 is 1. In certain embodiments, n7 is 1 and n1 is 1, n2 is 1, n3 is 1, n4 is 1, n5 is 1, and n6 is 1. In certain embodiments, n7 is 1 and n1 is 1, n2 is 0, n3 is 1, n4 is 0, n5 is 1, and n6 is 1. In certain embodiments, n7 is 2. In certain embodiments, n7 is 3.

In some embodiments, m6 is 1 and each of m1 through m5 are 1. In some embodiments, m6 is 1, m1 is 1, m2 is 0, m3 is 0, m4 is 1, and m5 is 0. In some embodiments, m6 is 1, m1 is 1, m2 is 1, m3 is 0, m4 is 1, and m5 is 0. In some embodiments, m6 is 1, m1 is 1, m2 is 0, m3 is 1, m4 is 1, and m5 is 0. In some embodiments, m6 is 1, m1 is 1, m2 is 0, m3 is 0, m4 is 1, and m5 is 1. In some embodiments, m6 is 2. In certain embodiments, m6 is 3.

In some embodiments, p6 is 1 and each of p1 through p5 is 1. In certain embodiments, p6 is 1, p1 is 1, p2 is 0, p3 is 0, p4 is 1, and p5 is 0. In some embodiments, p6 is 1, p1 is 1, p2 is 1, p3 is 0, p4 is 1, and p5 is 0. In some embodiments, p6 is 1, p1 is 1, p2 is 0, p3 is 1, p4 is 1, and p5 is 0. In some embodiments, p6 is 1, p1 is 1, p2 is 0, p3 is 0, p4 is 1, and p5 is 1. In some embodiments, p6 is 2 and at least one occurrence of p1 is 1, p2 is 1, p3 is 0, p4 is 1, and p5 is 0. In some embodiments, p6 is 2. In certain embodiments, p6 is 3.

In some embodiments, an amino acid element comprises one or more amino acids selected from the group consisting of glycine, alanine, serine, threonine, cysteine, valine, leucine, isoleucine, methionine, proline, phenylalanine, tyrosine, tryptophan, aspartic acid, glutamic acid, asparagine, glutamine, histidine, lysine, arginine, sarconsine, and beta-alanine.

In certain embodiments, an amino acid element is selected from the group consisting of glycine, sarcosine, beta-alanine, and glutamic acid.

In some embodiments, an amino acid element comprises a dipeptide, a tripeptide, a tetrapeptide, or a pentapeptide.

In more embodiments, an amino acid element has one of the following structures:

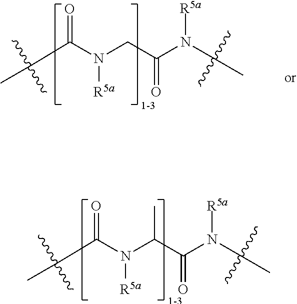

wherein:
each occurrence of R^{5a} is independently hydrogen, alkyl, hydroxyalkyl, or alkoxyalkyl.

In some embodiments, a charged element comprises moieties with a negative charge at pH 7.4 (i.e., a range from 6.3 to 8.5). In certain embodiments, a charged element comprises moieties with a positive charge at pH 7.4 (i.e., a range from 6.3 to 8.5).

In some embodiments, a charged element comprises one or more charged amino acid, one or more carboxylic acid, one or more sulfonic acid, one or more sulfonamide, one or more sulfate, one or more phosphate, one or more quaternary amine, one or more sulfamide, one or more sulfinimide, or combinations thereof.

In certain embodiments, a charged amino acid is aspartic acid, glutamic acid, histidine, lysine, or arginine.

In some embodiments, R¹, R², or R^{3a} comprises a non-cleavable linker (e.g., a linker, or segment thereof, that does not include a trigger element or immolative element).

In some embodiments, R¹, R², or R^{3a} comprises one of the following structures:

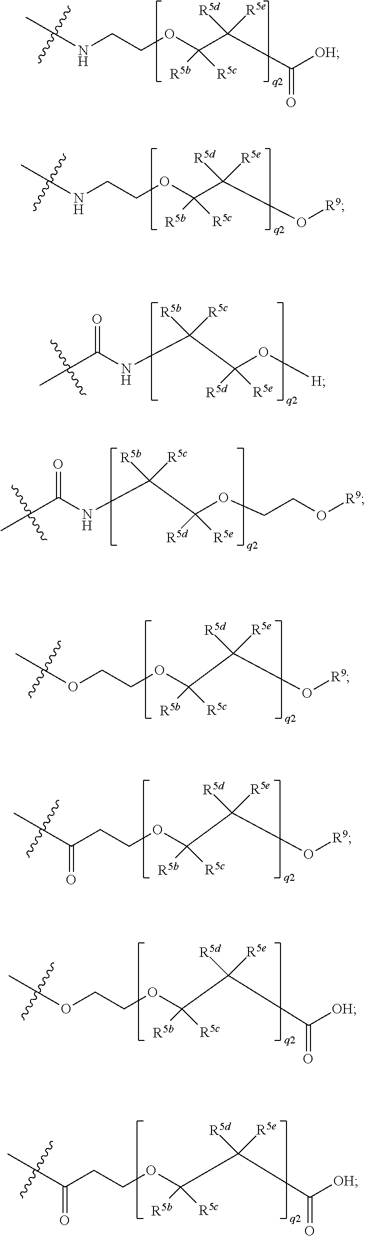

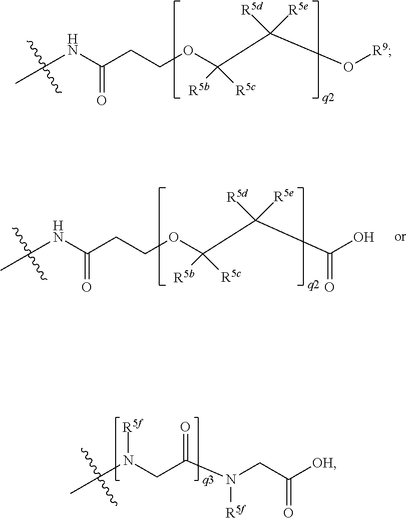

wherein:
each occurrence of R^{5b}, R^{5c} R^{5d}, and R^{5e} is independently selected from the group consisting of hydrogen, deuterium, alkyl, haloalkyl, halo, alkoxy, haloalkoxy, amino, hydroxyl, cyano, nitro, thiol, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, —P(O)₃H, alkyl-O—P(O)₃-alkyl, alkyl-P(O)₃-alkyl, —O-alkyl-S(O)₃-alkyl, —O-alkyl-O—P(O)₃-alkyl, —O-alkyl-P(O)₃-alkyl, —S(O)₃-alkyl, —OP(O)₃-alkyl, —P(O)₃-alkyl, sulfamide, sulfinimide;
each occurrence of R^{5f} is independently hydrogen, alkyl, hydroxyalkyl, or alkoxyalkyl;
each occurrence of R⁹ is independently hydrogen or alkyl;
each occurrence of q2 is independently an integer from 1-25; and
each occurrence of q3 is independently an integer from 5-15.

In some embodiments, a hydrophilic element comprises polyethylene glycol, polysarcosine, cyclodextrin, c-glycosides, or combinations thereof. In some embodiments, a hydrophilic element comprises one of the following structures:

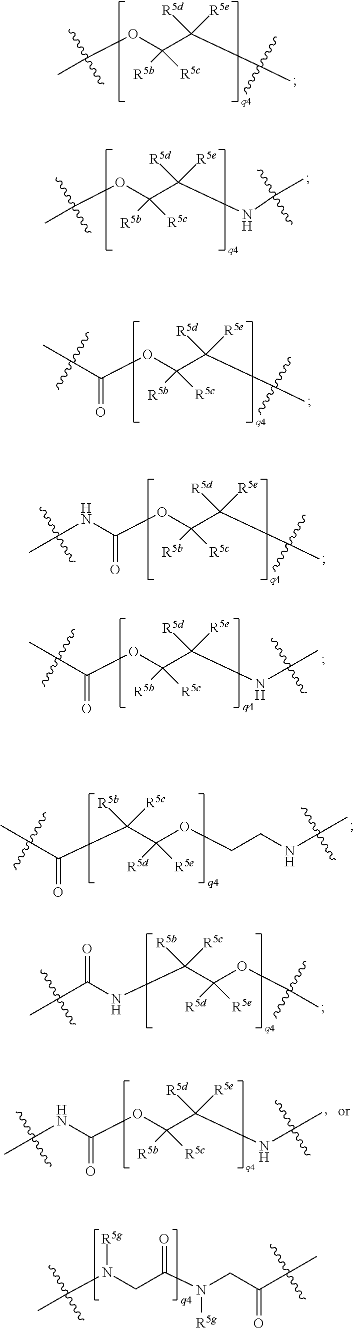

wherein:
each occurrence of R^{5b}, R^{5c} R^{5d}, and R^{5e} is independently selected from the group consisting of hydrogen, deuterium, alkyl, haloalkyl, halo, alkoxy, haloalkoxy, amino, hydroxyl, cyano, nitro, thiol, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, and —P(O)₃H,
each occurrence of R^{5g} is independently hydrogen, alkyl, hydroxyalkyl, or alkoxyalkyl; and
each occurrence of q4 is, independently an integer from 1-24.

In some embodiments, a hydrophilic element comprises one of the following structures:

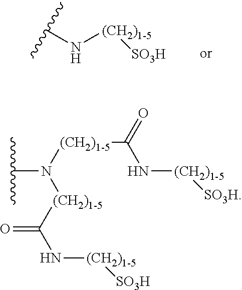 [SMILES: *NCS(=O)(=O)O ; *N(CC(=O)NCS(=O)(=O)O)CC(=O)NCS(=O)(=O)O]

In some embodiments, a hydrophilic element comprises one of the following structures:

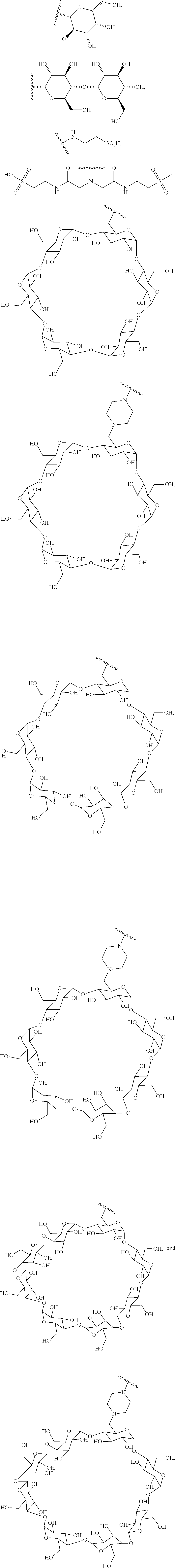

In some embodiments, a hydrophilic element has one of the following structures:

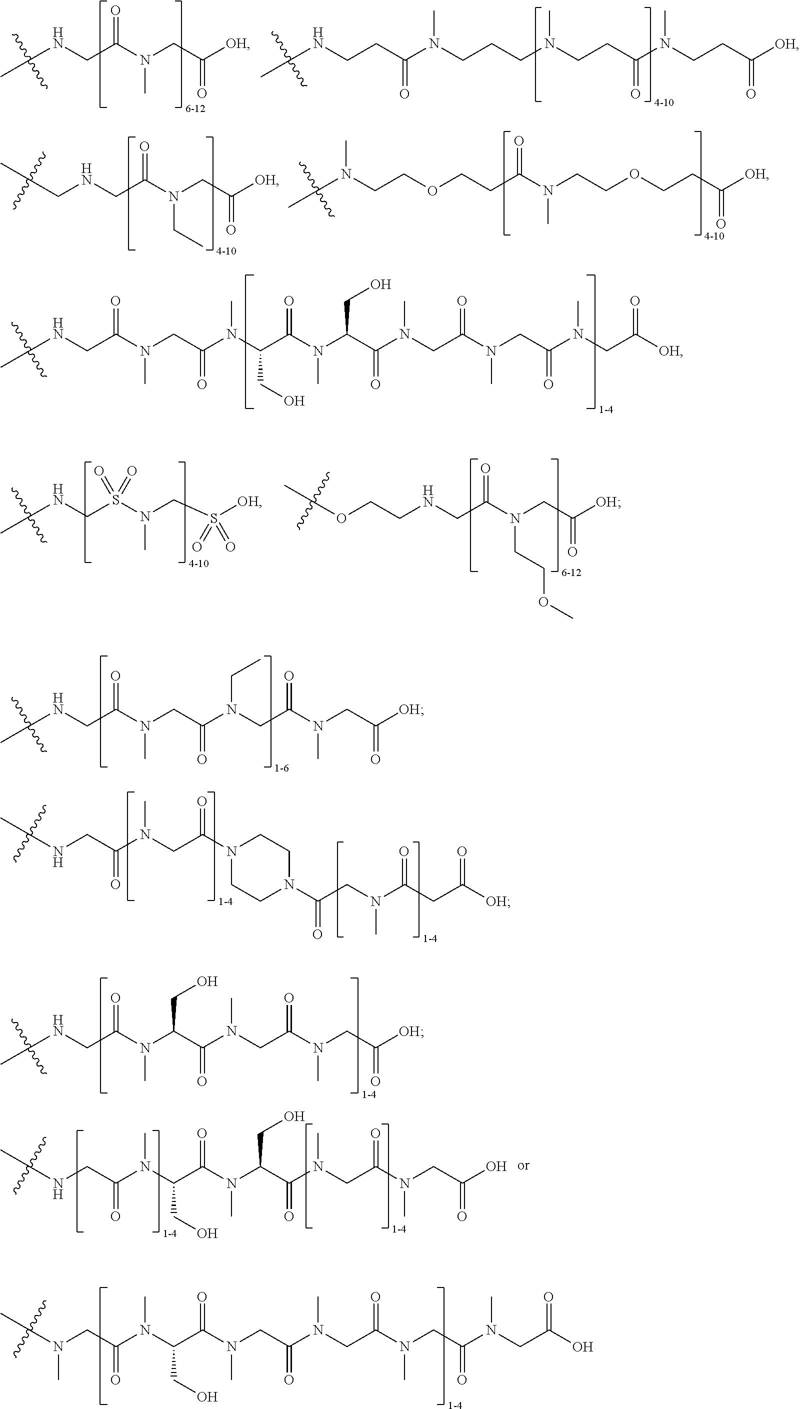

In some embodiments, a hydrophilic element has the following structure:

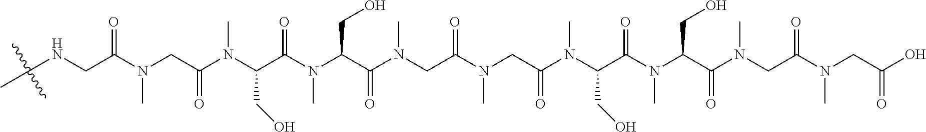 [SMILES: *NCC(=O)N(C)CC(=O)N(C)[C@@H](CO)C(=O)N(C)[C@@H](CO)C(=O)N(C)CC(=O)N(C)CC(=O)N(C)[C@@H](CO)C(=O)N(C)[C@@H](CO)C(=O)N(C)CC(=O)N(C)CC(=O)O]

In some embodiments, a hydrophilic element has one of the following structures:

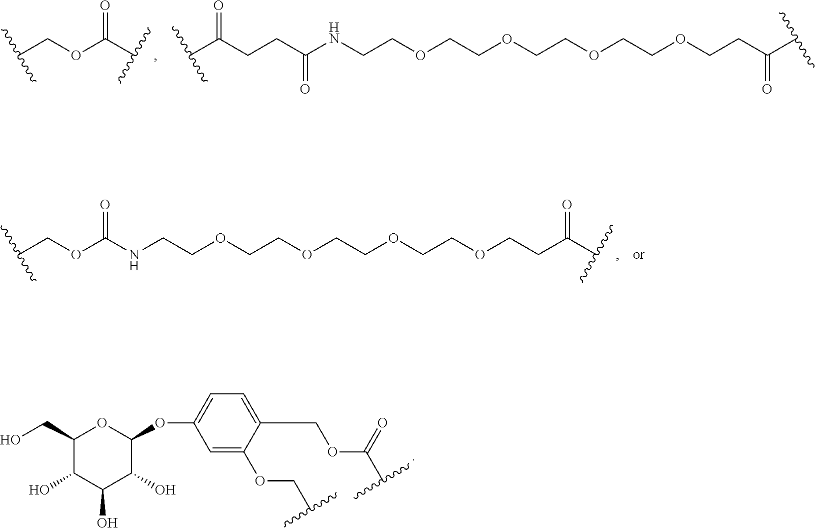 [SMILES: *COC(*)=O ; *C(=O)CCOCCOCCOCCOCCNC(=O)CCC(*)=O ; *COC(=O)NCCOCCOCCOCCOCCC(*)=O ; *COc1cc(O[C@@H]2O[C@H](CO)[C@@H](O)[C@H](O)[C@H]2O)ccc1COC(*)=O]

In some embodiments, a hydrophilic element has one of the following structures:

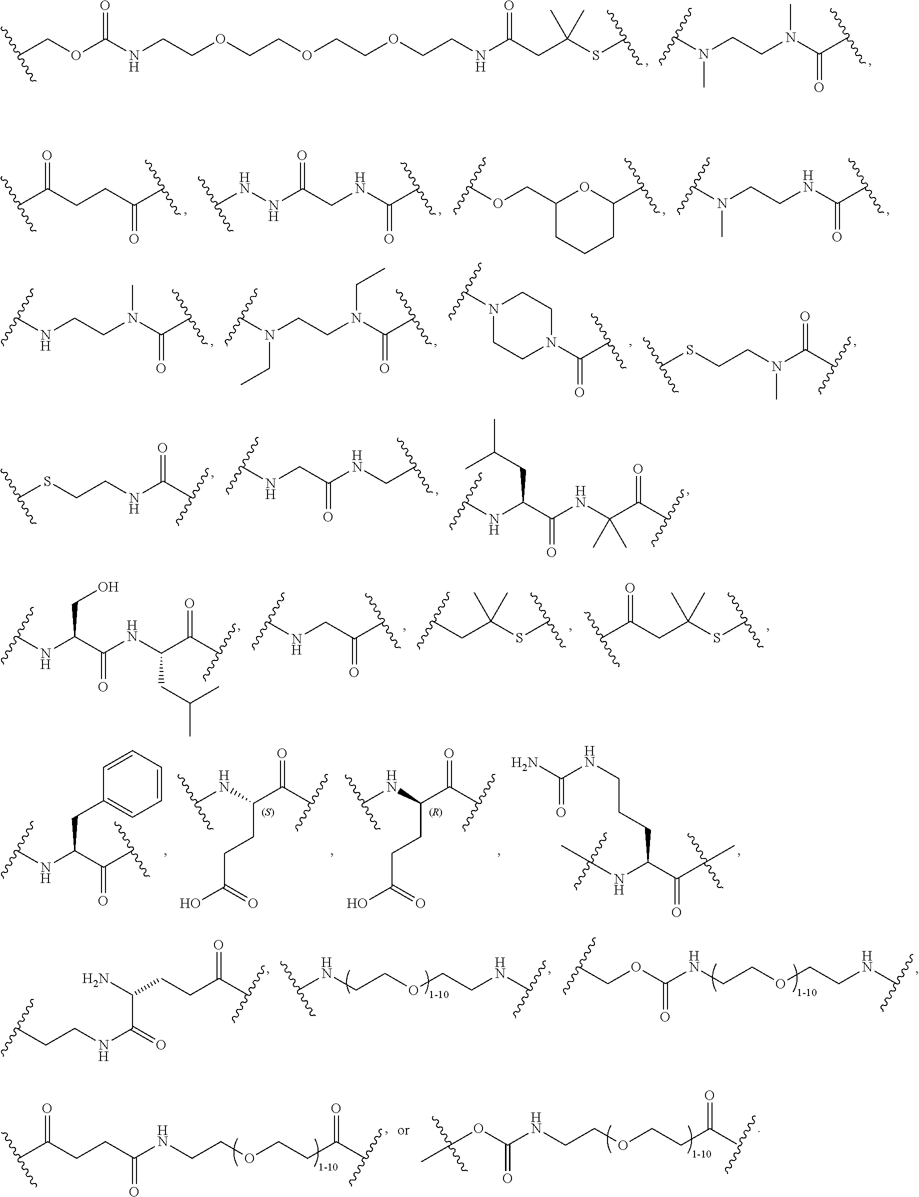

In some embodiments, a hydrophilic element comprises a polysarcosine. In some embodiments, a hydrophilic element is a polysarcosine comprising the following structure:

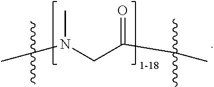

In some embodiments, a hydrophilic element is a polysarcosine with one of the following structures:

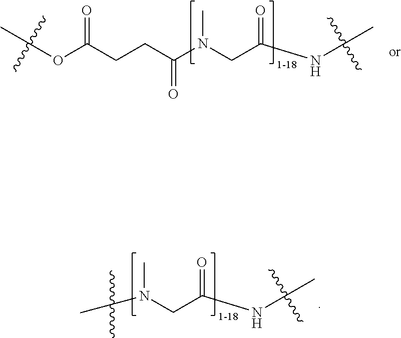

In some embodiments, a hydrophilic element has a molecular weight greater than 150 g/mole, greater than 200 g/mole, greater than 300 g/mole, greater than 400 g/mole, greater than 500 g/mole, greater than 600 g/mole, greater than 700 g/mole, greater than 800 g/mole, greater than 900 g/mole, or greater than 1000 g/mole. In some embodiments, a hydrophilic element has a molecular weight less than 150 g/mole, less than 200 g/mole, less than 300 g/mole, less than 400 g/mole, less than 500 g/mole, less than 600 g/mole, less than 700 g/mole, less than 800 g/mole, less than 900 g/mole, or less than 1000 g/mole.

In certain embodiments, L¹ is alkylene. In some embodiments, L¹ is C₁-C₆ alkylene. In certain embodiments, L² is alkylene. In some embodiments, L² is C₁-C₆ alkylene. In certain embodiments, L³ is alkylene. In some embodiments, L³ is C₁-C₆ alkylene.

In certain embodiments, L¹ is heteroalkylene. In some embodiments, L¹ is C₁-C₆ heteroalkylene (i.e., contains from 1-6 carbon atoms and one or more heteroatoms). In certain embodiments, L² is heteroalkylene. In some embodiments, L² is C₁-C₆ heteroalkylene. In certain embodiments, L³ is heteroalkylene. In some embodiments, L³ is C₁-C₆ heteroalkylene.

In more embodiments, L¹, L², or L³ are C₁-C₆ heteroalkylene and contain heteroatoms selected form O and N. In some embodiments, L³ is a direct bond.

In more embodiments, L¹, L², or L³ have one of the following structures:

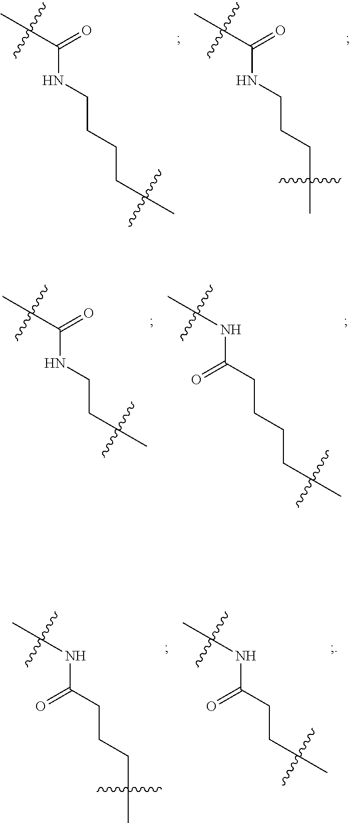 [SMILES: *CCCCNC(*)=O ; *CCCNC(*)=O ; *CCNC(*)=O ; *CCCCC(=O)N* ; *CCCC(=O)N* ; *CCC(=O)N*]

In some embodiments, a trigger element comprises a dipeptide, a tripeptide, a tetrapeptide, a pentapeptide, a glucuronide, a disulfide, a phosphate, a diphosphate, a triphosphate, a hydrazone, or combinations thereof. In some other embodiments, a trigger element comprises beta-glucuronic acid. In certain embodiments, a trigger element comprises a dipeptide, a tripeptide, a tetrapeptide, or a pentapeptide. In some embodiments, a trigger element comprises two or more amino acids selected from the group consisting of valine, citrulline, alanine, glycine, phenylalanine, lysine, or combinations thereof. In certain embodiments, a trigger element comprises a sequence of amino acids selected from the group consisting of valine-citrulline, valine-alanine, glycine-glycine-phenylalanine-glycine, and combinations thereof. In some embodiments, a trigger element comprises one of the following structures, including combinations thereof:

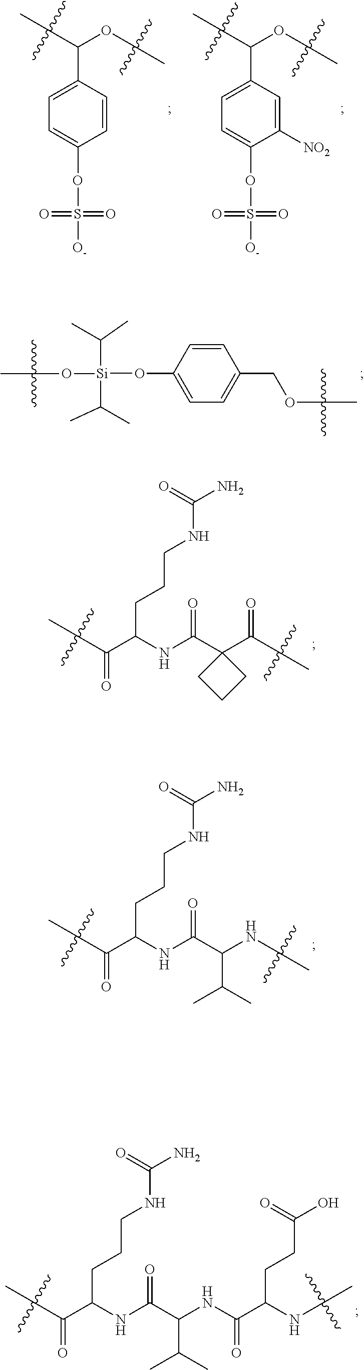

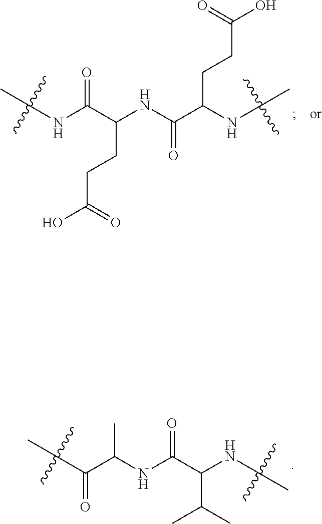 [SMILES: *NC(=O)C(CCC(=O)O)NC(=O)C(CCC(=O)O)N* ; *NC(C(=O)NC(C)C(*)=O)C(C)C]

In some embodiments, a trigger element has a molecular weight greater than 150 g/mole, greater than 200 g/mole, greater than 300 g/mole, greater than 400 g/mole, greater than 500 g/mole, greater than 600 g/mole, greater than 700 g/mole, greater than 800 g/mole, greater than 900 g/mole, or greater than 1000 g/mole. In some embodiments, a trigger element has a molecular weight less than 150 g/mole, less than 200 g/mole, less than 300 g/mole, less than 400 g/mole, less than 500 g/mole, less than 600 g/mole, less than 700 g/mole, less than 800 g/mole, less than 900 g/mole, or less than 1000 g/mole.

In some embodiments, a trigger element has the following structure:

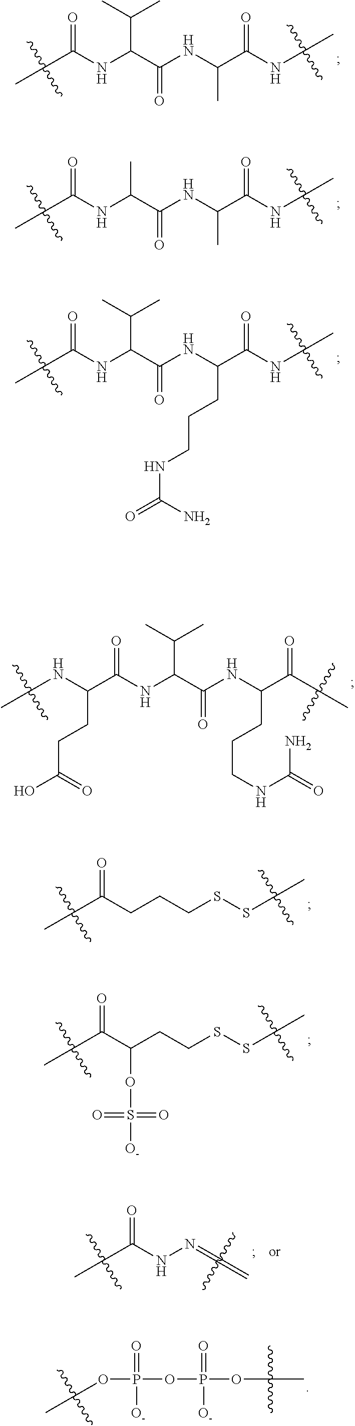

In some embodiments, a trigger element is specifically cleaved by an enzyme. For example, a trigger element can be cleaved by a lysosomal enzyme. A trigger element can be peptide-based or can include peptidic regions that can act as substrates for enzymes. Peptide based trigger elements can be more stable in plasma and extracellular milieu than chemically labile linkers.

Exemplary disulfide-containing trigger elements can include the following structures:

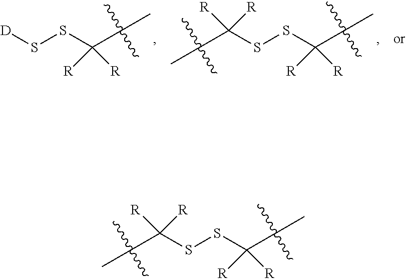

wherein D is a payload and R is independently selected at each occurrence from, for example, hydrogen or C₁-C₆ alkyl. Increasing steric hindrance adjacent to the disulfide bond can increase the stability of the linker. The above structures can result in increased in vivo stability when one or more R groups is selected from a lower alkyl, such as methyl.

Peptide bonds can have good serum stability, as lysosomal proteolytic enzymes can have very low activity in blood due to endogenous inhibitors and the unfavorably high pH value of blood compared to lysosomes. Release of a payload from conjugate of Structure (II) can occur due to the action of lysosomal proteases, e.g., cathepsin and plasmin. These proteases can be present at elevated levels in certain tumor tissues. A trigger element can be cleavable by a lysosomal enzyme. The lysosomal enzyme can be, for example, cathepsin B, β-glucuronidase, or β-galactosidase.

A cleavable peptide of a trigger element can be selected from tetrapeptides such as Gly-Phe-Leu-Gly, Ala-Leu-Ala-Leu, tripeptides such as Glu-Val-Cit, or dipeptides such as Val-Cit, Val-Ala, Ala-Ala, and Phe-Lys. Dipeptides can have lower hydrophobicity compared to longer peptides.

In some embodiments, a trigger element may be a single amino acid residue. In some embodiments, the trigger element comprises Asn (e.g., a legumain cleavable)

Enzymatically cleavable trigger elements be combined with an immolative element to provide additional spatial separation between a payload and the site of enzymatic cleavage. The direct attachment of payload to a peptidic trigger element can result in proteolytic release of a payload or of an amino acid adduct of a payload thereby impairing its activity. The use of an immolative element can allow for the release of the fully active, chemically unmodified payload upon amide bond hydrolysis.

A trigger element can contain a chemically labile group such as hydrazone and/or disulfide groups. A trigger element comprising chemically labile group or groups can exploit differential properties between the plasma and some cytoplasmic compartments. The intracellular conditions that can facilitate release of a payload for hydrazone containing trigger elements can be the acidic environment of endosomes and lysosomes, while the disulfide containing trigger elements can be reduced in the cytosol, which can contain high thiol concentrations, e.g., glutathione. The plasma stability of a trigger element containing a chemically labile group can be increased by introducing steric hindrance using substituents near the chemically labile group.

Acid-labile groups, such as hydrazone, can remain intact during systemic circulation in the blood's neutral pH environment (pH 7.3-7.5) and can undergo hydrolysis and can release a payload once the conjugate of Structure (II) is internalized into mildly acidic endosomal (pH 5.0-6.5) and lysosomal (pH 4.5-5.0) compartments of the cell. This pH dependent release mechanism can be associated with non-specific release of a payload. To increase the stability of a hydrazone group of a trigger element, a trigger element can be varied by chemical modification, e.g., substitution, allowing tuning to achieve more efficient release in the lysosome with a minimized loss in circulation.

In some embodiments, a trigger element comprises a hydrazone moiety having one of the following structures:

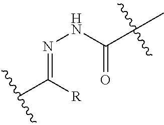

wherein R is selected from C₁-C₆ alkyl, aryl, and —O—C₁-C₆ alkyl.

Hydrazone-containing trigger elements can contain additional cleavage sites, such as additional acid-labile cleavage sites and/or enzymatically labile cleavage sites (e.g., a disulfide). Conjugates and compounds including exemplary hydrazone-containing trigger elements can include, for example, the following structure:

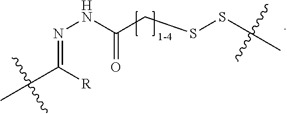

wherein R is selected from C₁-C₆ alkyl, aryl, and —O—C₁-C₆ alkyl.

Other acid-labile groups that can be included in trigger elements include cis-aconityl-containing linkers. cis-Aconityl chemistry can use a carboxylic acid juxtaposed to an amide bond to accelerate amide hydrolysis under acidic conditions.

Trigger elements can also include a disulfide group. Disulfides can be thermodynamically stable at physiological pH and release a payload upon internalization of the conjugate of Structure (II) into cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing trigger element can be reasonably stable in circulation, selectively releasing a payload in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing trigger element can be enhanced by chemical modification of a trigger element, e.g., use of steric hindrance adjacent to the disulfide bond.

A trigger element can also be a β-glucuronic acid-based linker. Facile release of a payload, can be realized through cleavage of the β-glucuronide glycosidic bond by the lysosomal enzyme β-glucuronidase. This enzyme can be abundantly present within lysosomes and can be overexpressed in some tumor types, while the enzyme activity outside cells can be low. β-Glucuronic acid-based linkers can be used to circumvent the tendency of a conjugate to undergo aggregation due to the hydrophilic nature of β-glucuronides. In some embodiments, a trigger element comprises a β-glucuronic acid.

The following scheme depicts the release of a payload (D) from a conjugate of Structure (II) containing a β-glucuronic acid-based trigger element:

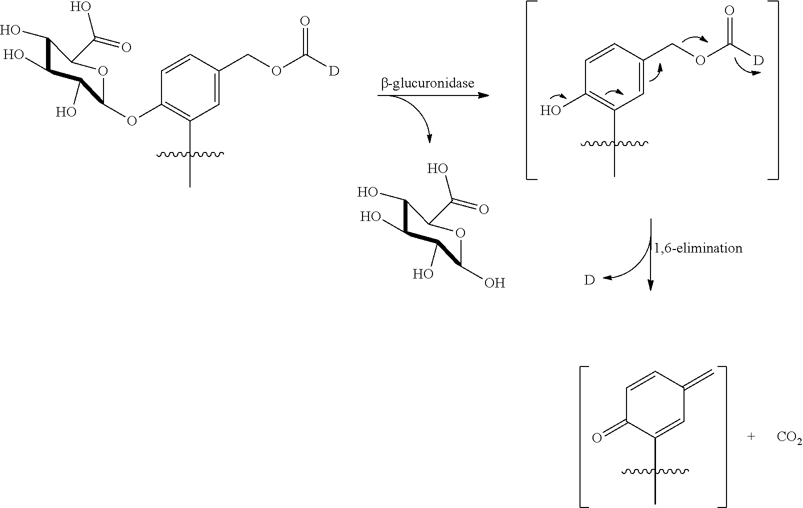

A variety of cleavable β-glucuronic acid-based linkers useful for linking drugs such as auristatins, camptothecin analogues, doxorubicin analogues, CBI minor-groove binders, and psymberin to antibodies have been described. Accordingly, these β-glucuronic acid-based trigger elements are used in the conjugates of Structure (II). In some embodiments, a trigger element comprises a β-galactoside-based linker. β-Galactoside is present abundantly within lysosomes, while the enzyme activity outside cells is low.

A trigger element may include one or more peptides. In some embodiments, a peptide can be selected to contain natural amino acids, unnatural amino acids, or any combination thereof. In some embodiments, a peptide can be a tripeptide or a dipeptide. In particular embodiments, a dipeptide comprises L-amino acids, such as Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

Trigger elements and immolative groups are known in the art, for example in International Application No. PCT/US2021/054296; the trigger elements and immolative elements of which are hereby incorporated by reference in their entirety.

One immolative element can be a bifunctional para-aminobenzyl alcohol group, which can link to a trigger element through an amino group, forming an amide bond, while an amine containing payload can be attached through carbamate functionalities to the benzylic hydroxyl group of the para-aminobenzyl alcohol (to give a p-amidobenzylcarbamate. The resulting pro-compound can be activated upon protease-mediated cleavage, leading to a 1,6-elimination reaction releasing the unmodified payload and remnants of the antibody-linker.

In some embodiments, an immolative element comprises para-aminobenzyloxycarbonyl, an aminal, a hydrazine, a disulfide, an amide, an ester, a hydrazine, a phosphotriester, a diester, a β-glucuronide, a double bond, a triple bond, an ether bond, a ketone, a diol, a cyano, a nitro, a quaternary amine, or combinations thereof. In certain embodiments, an immolative element comprises a paramethoxybenzyl, a dialkyldialkoxysilane, a diaryldialkoxysilane, an orthoester, an acetal, an optionally substituted β-thiopropionate, a ketal, a phosphoramidate, a hydrazone, a vinyl ether, an imine, an aconityl, a trityl, a polyketal, a bis-arylhydrazone, a diazobenzene, a vivinal diol, a pyrophosphate diester, or combinations thereof.

In certain embodiments, an immolative element comprises one of the following structures:

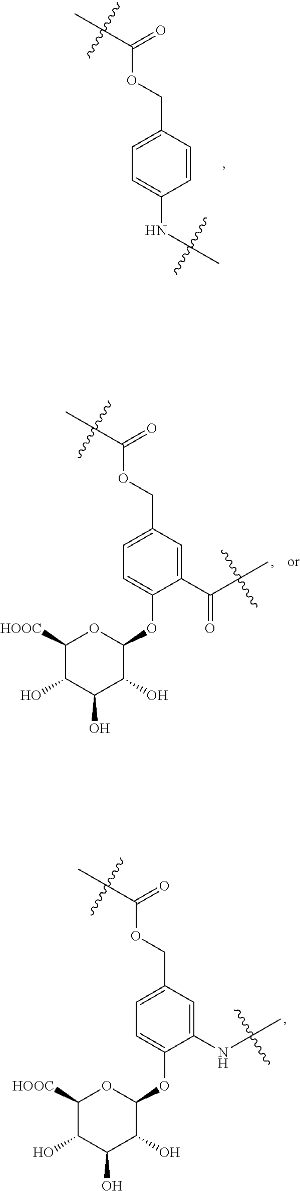 [SMILES: *Nc1ccc(COC(*)=O)cc1 ; *C(=O)OCc1ccc(O[C@@H]2O[C@H](C(=O)O)[C@@H](O)[C@H](O)[C@H]2O)c(C(*)=O)c1 ; *Nc1cc(COC(*)=O)ccc1O[C@@H]1O[C@H](C(=O)O)[C@@H](O)[C@H](O)[C@H]1O]

In some embodiments, an immolative element comprises the following structure:

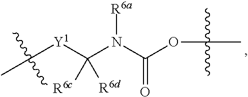

wherein:
R^{6a}, R^{6b}, R^{6c}, and R^{6d} are independently hydrogen, an optionally substituted alkyl, an optionally substituted aryl, or optionally substituted heteroaryl, or
R^{6a} and R^{6c} together with the nitrogen and carbon atoms to which they are attached form azetidinyl, pyrrolodinyl, piperidinyl, or homopiperidinyl and R^{6d} is hydrogen; and
Y¹ is —O—, —S—, or —NR^{6b}—;

In certain embodiments, an immolative element comprises the following structure:

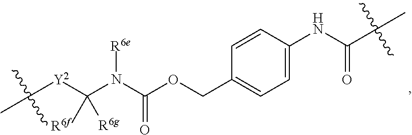

wherein:
R^{6e}, R^{6f}, R^{6g}, and R^{6h} are independently hydrogen, an optionally substituted alkyl, an optionally substituted aryl, or optionally substituted heteroaryl, or
R^{6a} and R^{6c} together with the nitrogen and carbon atoms to which they are attached form azetidinyl, pyrrolodinyl, piperidinyl, or homopiperidinyl and R^{6d} is hydrogen; and
Y² is —O—, —S—, or —NR^{6f}—;

In some embodiments, an immolative element comprises the following structure:

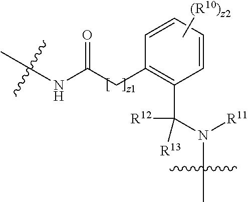

wherein:
each occurrence of R¹⁰ is independently alkyl, alkoxy, or halo;
R¹¹ is hydrogen, alkyl, or —(CH₂CH₂O)_{z3}—CH₃;
R¹² is hydrogen or alkyl;
R¹³ is hydrogen or alkyl;
z1 is 0 or 1;
z2 is 0, 1, 2, 3, or 4; and
z3 is 0, 1, 2, 3, 4, 5, 6, 7, 8, 9, or 10; or

In some embodiments, an immolative element comprises one of the following structures:

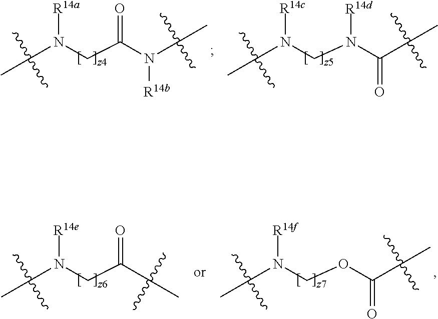

wherein:
R^{14a}, R^{14b}, R^{14c}, R^{14d}, R^{14e}, and R^{14f} are each independently hydrogen, alkyl, hydroxyalkyl, or alkoxyalkyl;
z4, z5, z6, and z7 are each independently 1, 2, 3, 4, 5, or 6;

In some embodiments, an immolative element comprises one of the following structures:

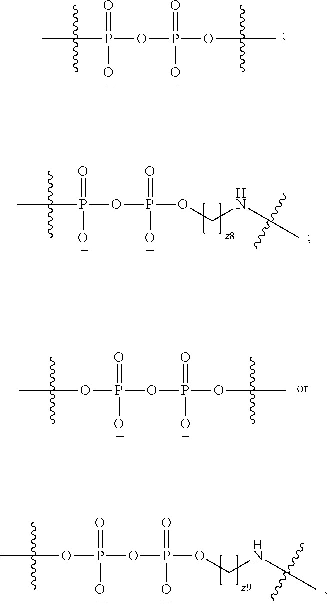

wherein:
z8 and z9 are each independently 1, 2, 3, 4, 5, or 6; or

In certain embodiments, an immolative element comprises one of the following structures:

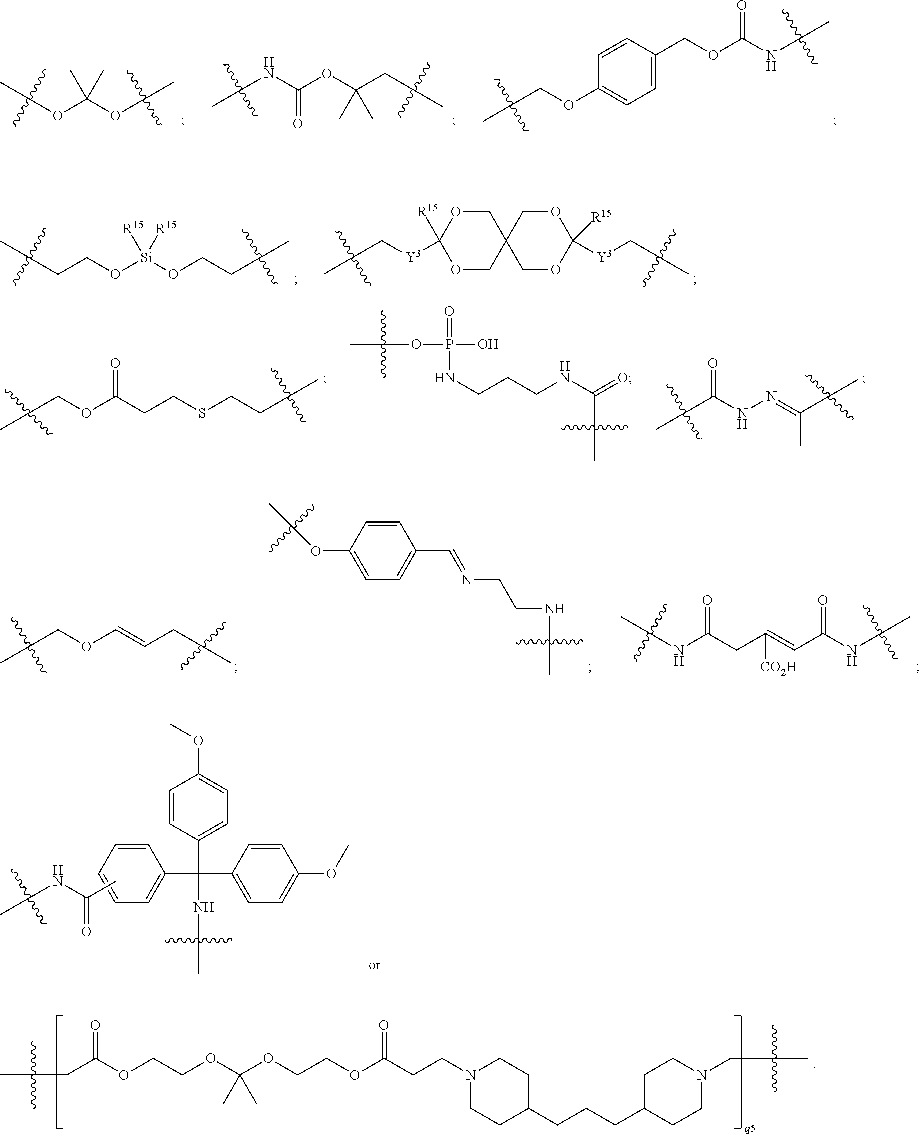

wherein:
each occurrence of R¹⁵ is independently H, methyl, ethyl, isopropyl, tert-butyl, or phenyl;
Y³ is O or CH₂; and
q5 is an integer ranging from 1-5.

In some embodiments, an immolative element has a molecular weight greater than 150 g/mole, greater than 200 g/mole, greater than 300 g/mole, greater than 400 g/mole, greater than 500 g/mole, greater than 600 g/mole, greater than 700 g/mole, greater than 800 g/mole, greater than 900 g/mole, or greater than 1000 g/mole. In some embodiments, an immolative element has a molecular weight less than 150 g/mole, less than 200 g/mole, less than 300 g/mole, less than 400 g/mole, less than 500 g/mole, less than 600 g/mole, less than 700 g/mole, less than 800 g/mole, less than 900 g/mole, or less than 1000 g/mole.

In some embodiments, an immolative element and a trigger element together have the following structure:

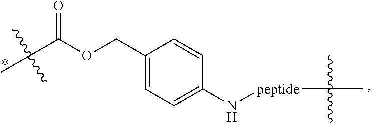

wherein a trigger element is denoted with “peptide” and comprises from one to ten amino acids, and * [symbol] represents the point of attachment to a payload. In some embodiments, the peptide comprises Val-Cit or Val-Ala. Heterocyclic variants (e.g., pyridinyl, pyrimidinyl, etc.) of this immolative element may also be used.

In some embodiments, an immolative element contains a phenol group that is covalently bound to the remainder of the molecule through the phenolic oxygen. One such immolative element relies on a methodology in which a diamino-ethane “Space Link” is used in conjunction with traditional “PABO”-based immolative element to deliver phenols.

In some embodiments, a trigger element can include non-cleavable portions or segments. Polyethylene glycol (PEG) and related polymers can be included with cleavable groups such as a disulfide, a hydrazone or a dipeptide to form an immolative group and/or trigger element.

Other degradable linkages that can be included in immolative elements can include esters. Esters can be formed by the reaction of PEG carboxylic acids or activated PEG carboxylic acids with alcohol groups on a payload such ester groups can hydrolyze under physiological conditions to release a payload. Other hydrolytically degradable linkages can include carbonate linkages, imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including at the end of a polymer, and a 5′ hydroxyl group of an oligonucleotide.

In some embodiments, a trigger element, immolative group, and payload together have the following structure:

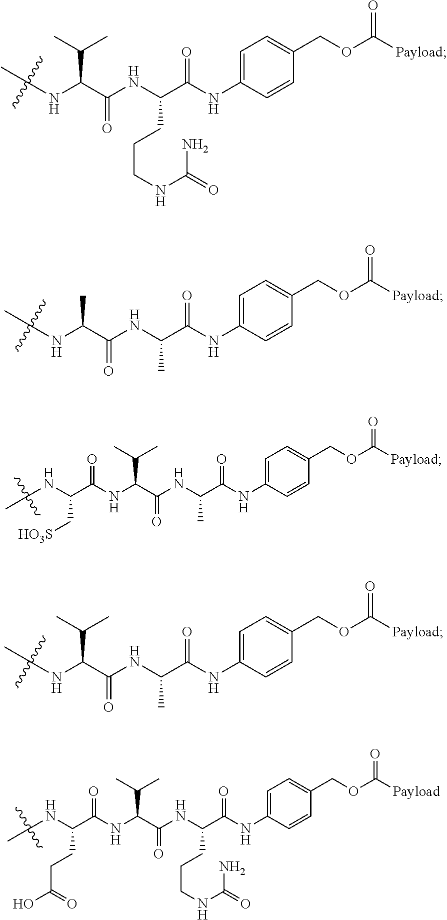

wherein [symbol] indicates an attachment site to the remainder of the molecule (i.e., a compound of Structure (I) or conjugate of Structure (II)) and a payload is indicated with text. Amino acids of embodiments above may be replaced or used in addition to other amino acids, in some embodiments, a trigger element is Asn-Cit, Arg-Cit, Val-Glu, Ser-Cit, Lys-Cit, Asp-Cit, Phe-Lys, Glu-Val-Cit, Glu-Val-Cit, Glu-Glu-Val-Cit, or Glu-Glu-Glu-Val-Cit, and an immolative element is PABC.

In some embodiments, the phenyl portion of the PABC is substituted with one or more substituents. In some embodiments, the substituents have one of the following structures:

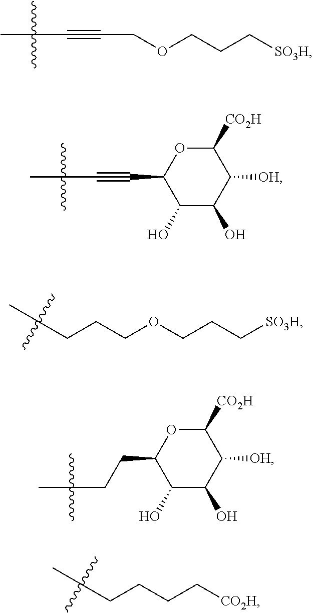 [SMILES: *C#CCOCCCS(=O)(=O)O ; *C#C[C@H]1O[C@@H](C(=O)O)[C@H](O)[C@@H](O)[C@@H]1O ; *CCCOCCCS(=O)(=O)O ; *CC[C@H]1O[C@@H](C(=O)O)[C@H](O)[C@@H](O)[C@@H]1O ; *CCCCC(=O)O]

In some embodiments, an immolative group comprises one of the following structures:

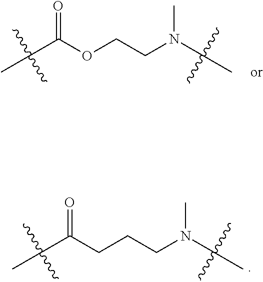

In some embodiments, a trigger element, an immolative element, and a payload together have one of the following structures, wherein a payload is represented with text:

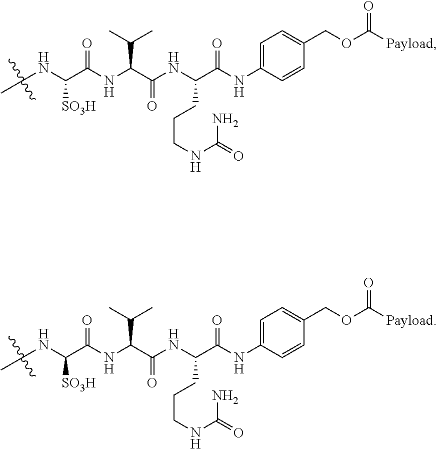

In some embodiments, an immolative element has the following structure:

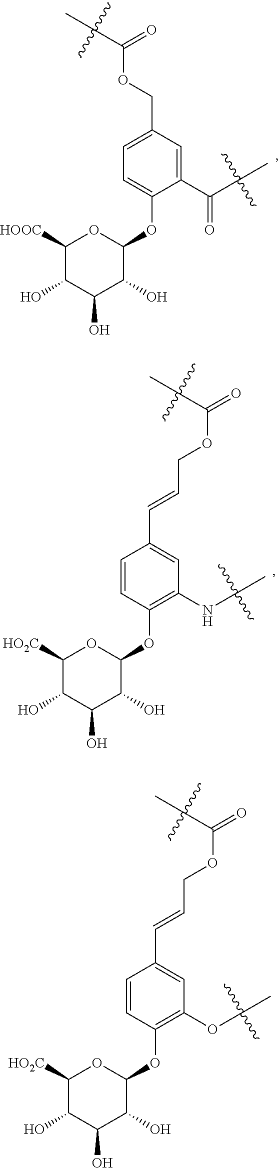 [SMILES: *C(=O)OCc1ccc(O[C@@H]2O[C@H](C(=O)O)[C@@H](O)[C@H](O)[C@H]2O)c(C(*)=O)c1 ; *Nc1cc(/C=C/COC(*)=O)ccc1O[C@@H]1O[C@H](C(=O)O)[C@@H](O)[C@H](O)[C@H]1O ; *Oc1cc(/C=C/COC(*)=O)ccc1O[C@@H]1O[C@H](C(=O)O)[C@@H](O)[C@H](O)[C@H]1O]

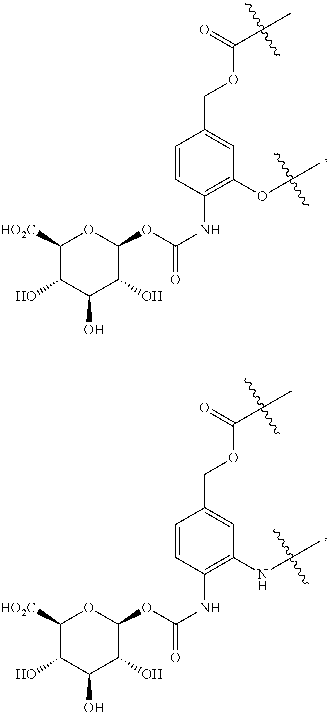 [SMILES: *Oc1cc(COC(*)=O)ccc1NC(=O)O[C@@H]1O[C@H](C(=O)O)[C@@H](O)[C@H](O)[C@H]1O ; *Nc1cc(COC(*)=O)ccc1NC(=O)O[C@@H]1O[C@H](C(=O)O)[C@@H](O)[C@H](O)[C@H]1O]

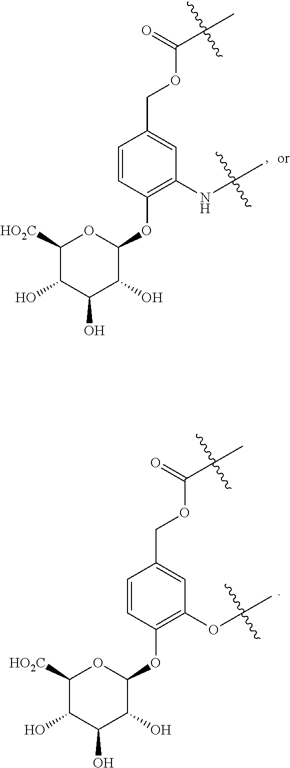 [SMILES: *Nc1cc(COC(*)=O)ccc1O[C@@H]1O[C@H](C(=O)O)[C@@H](O)[C@H](O)[C@H]1O ; *Oc1cc(COC(*)=O)ccc1O[C@@H]1O[C@H](C(=O)O)[C@@H](O)[C@H](O)[C@H]1O]

The structures above show a substitution pattern of 1, 3, 4 on the phenyl ring of an immolative element. In some embodiments, a substitution pattern may be 1, 2, 4 (i.e., 1 being a linkage to a payload, 2 being a linkage to the remainder of the molecule and 4 being a linkage to the carbohydrate) or 1, 3, 5 (i.e., 1 being a linkage to a payload, 3 being a linkage to the remainder of the molecule and 4 being a linkage to the carbohydrate).

Although cleavable linker (e.g., linkers with trigger elements or immolative elements) can provide certain advantages, linkers need not be cleavable. For non-cleavable linkers, a payload release may not depend on the differential properties between the plasma and some cytoplasmic compartments. The release of a payload can occur after internalization of the conjugate of Structure (II) via antigen-mediated endocytosis and delivery to lysosomal compartment, where the targeting moiety (or binding fragment thereof) can be degraded to the level of amino acids through intracellular proteolytic degradation. This process can release a payload or payload derivative. A payload or payload derivative can be more hydrophilic and less membrane permeable, which can lead to less bystander effects and less non-specific toxicities compared to conjugates with a cleavable linker. Conjugates with non-cleavable linkers can have greater stability in circulation than conjugates with cleavable linkers. Non-cleavable linkers can include alkylene chains, or can be polymeric, such as, for example, based upon polyalkylene glycol polymers, amide polymers, or can include segments of alkylene chains, polyalkylene glycols and/or amide polymers. The linker can contain a polyethylene glycol segment having from 1 to 6 ethylene glycol units. In some embodiments, -L¹-R¹ or L²-R² comprises a linker that is non-cleavable in vivo.

In some embodiments, a trigger element and an immolative element together comprise one of the following structures:

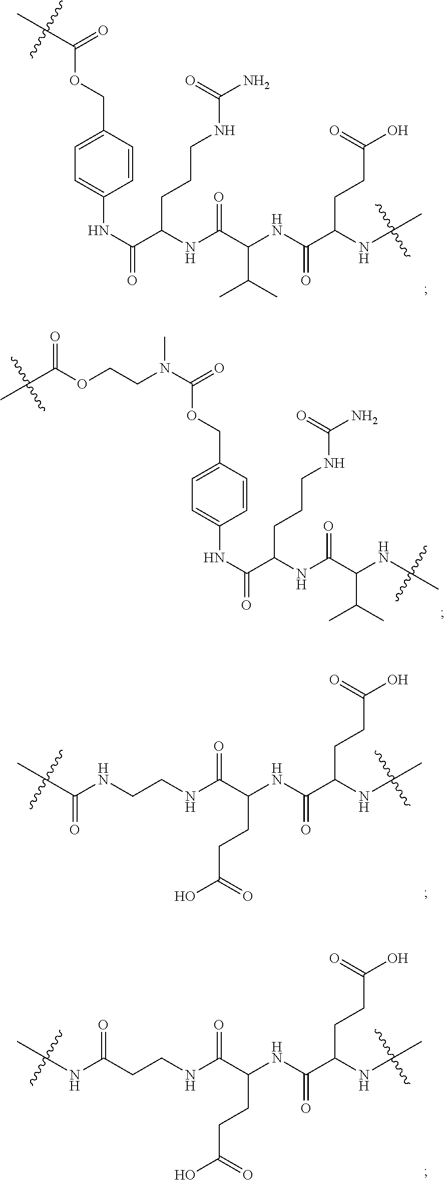 [SMILES: *NC(CCC(=O)O)C(=O)NC(C(=O)NC(CCCNC(N)=O)C(=O)Nc1ccc(COC(*)=O)cc1)C(C)C ; *NC(C(=O)NC(CCCNC(N)=O)C(=O)Nc1ccc(COC(=O)N(C)CCOC(*)=O)cc1)C(C)C ; *NC(CCC(=O)O)C(=O)NC(CCC(=O)O)C(=O)NCCNC(*)=O ; *NC(=O)CCNC(=O)C(CCC(=O)O)NC(=O)C(CCC(=O)O)N*]

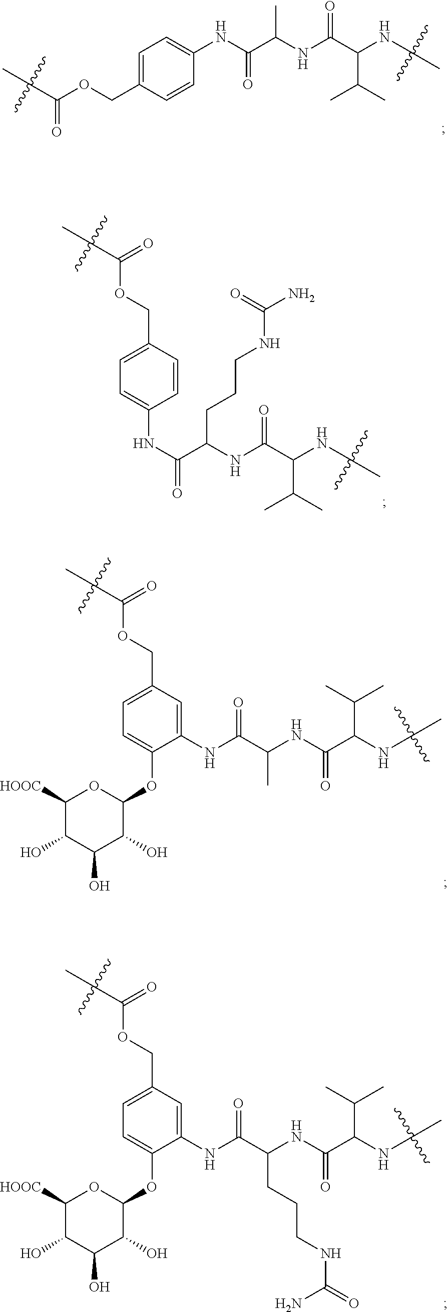 [SMILES: *NC(C(=O)NC(C)C(=O)Nc1ccc(COC(*)=O)cc1)C(C)C ; *NC(C(=O)NC(CCCNC(N)=O)C(=O)Nc1ccc(COC(*)=O)cc1)C(C)C ; *NC(C(=O)NC(C)C(=O)Nc1cc(COC(*)=O)ccc1O[C@@H]1O[C@H](C(=O)O)[C@@H](O)[C@H](O)[C@H]1O)C(C)C ; *NC(C(=O)NC(CCCNC(N)=O)C(=O)Nc1cc(COC(*)=O)ccc1O[C@@H]1O[C@H](C(=O)O)[C@@H](O)[C@H](O)[C@H]1O)C(C)C]

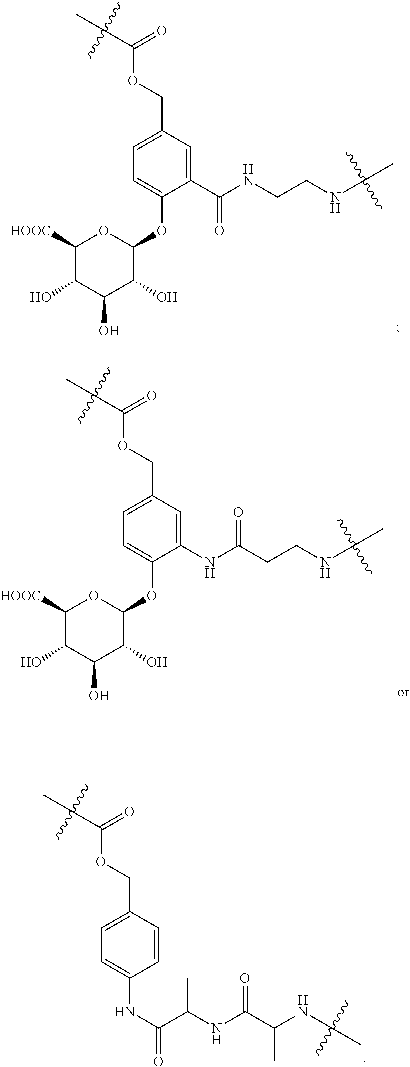 [SMILES: *NCCNC(=O)c1cc(COC(*)=O)ccc1O[C@@H]1O[C@H](C(=O)O)[C@@H](O)[C@H](O)[C@H]1O ; *NCCC(=O)Nc1cc(COC(*)=O)ccc1O[C@@H]1O[C@H](C(=O)O)[C@@H](O)[C@H](O)[C@H]1O ; *NC(C)C(=O)NC(C)C(=O)Nc1ccc(COC(*)=O)cc1]

In some embodiments, a heteroalkylene element comprises polyethylene glycol or polypropylene glycol. In some embodiments, a heteroalkylene element comprises one of the following structures:

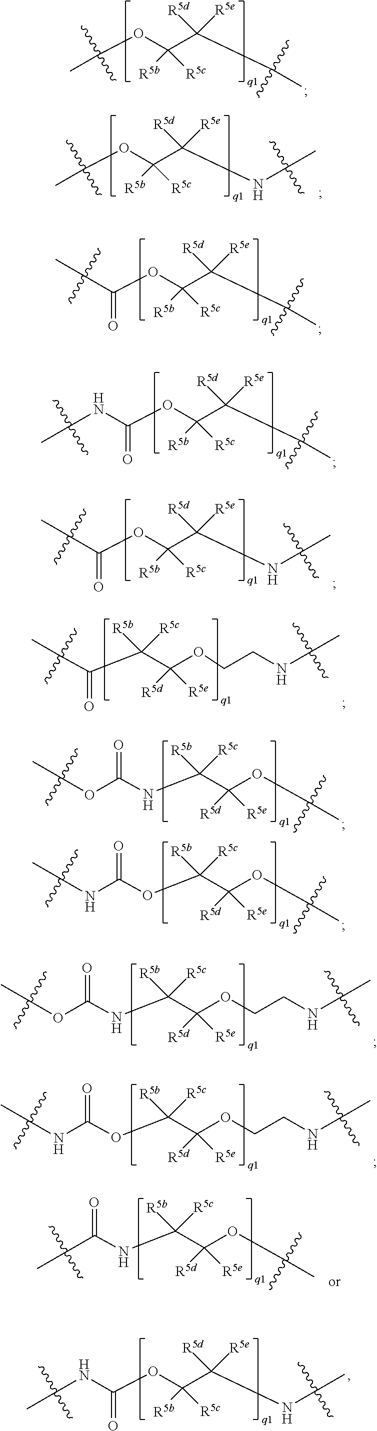

wherein:
each occurrence of R^{5b}, R^{5c} R^{5d}, and R^{5e} is independently selected from the group consisting of hydrogen, deuterium, alkyl, haloalkyl, halo, alkoxy, haloalkoxy, amino, hydroxyl, cyano, nitro, thiol, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, and —P(O)₃H,
each occurrence of q1 is, independently an integer from 1-24.

In some embodiments, R^{5b}, R^{5c} R^{5d}, and R^{5e} are all hydrogen.

In some embodiments, a polar cap comprises one or more charged amino acid, one or more polyol, or combinations thereof. In certain embodiments, a polar cap comprises a diol, a triol, a tetraol, or combinations thereof. In some embodiments, a polar cap comprises glycerol, trimethylolpropane, pentaerythritol, maltitol, sorbitol, xylitol, erythritol, isomalt, or combinations thereof. In certain embodiments, a polar cap comprises one or more natural amino acids. In some embodiments, a polar cap comprises one or more non-natural amino acids. In some embodiments, a polar cap comprises one or more non-natural amino acids and one or more natural amino acids. In certain embodiments, a polar cap comprises serine, threonine, cysteine, proline, asparagine, glutamine, lysine, arginine, histidine, aspartate, glutamate, 4-hydroxyproline, 5-hydroxylysine, homoserine, homocysteine, ornithine, beta-alanine, statine, or gamma aminobutyric acid. In certain embodiments, a polar cap comprises aspartic acid, serine, glutamic acid, serine-beta-glucose, or combinations thereof.

In some embodiments, a polar cap comprises one of the following structures:

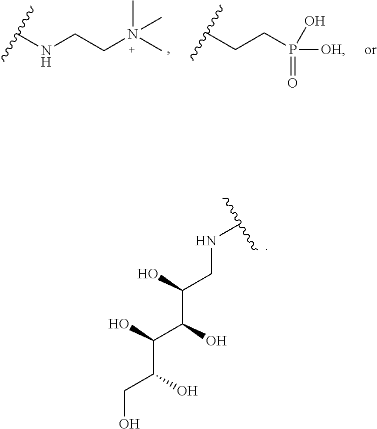

In more embodiments, a polar cap has one of the following structures, including combinations thereof:

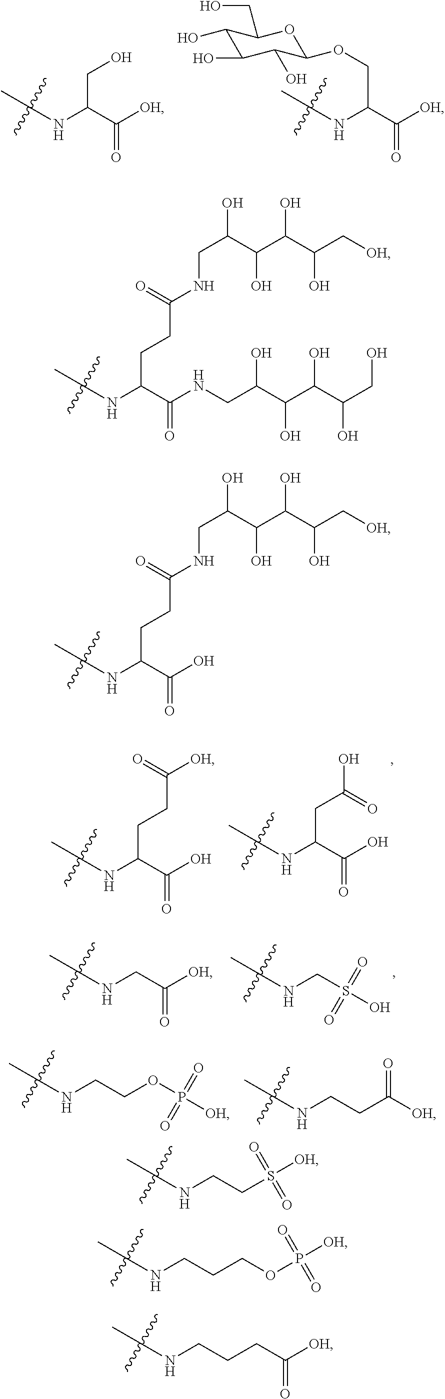

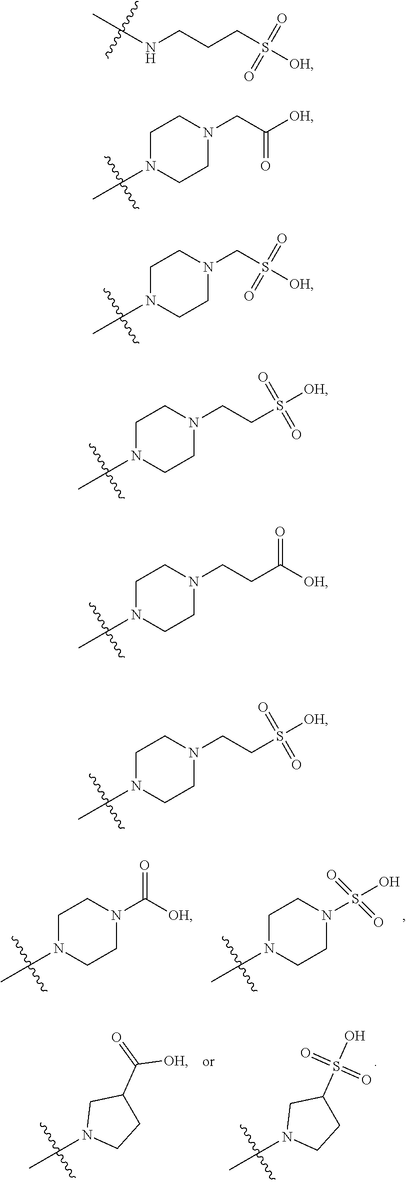

In more embodiments, L¹, L², or L³ comprise a linker selected from the group alkylene, alkylene-L^{a}-, alkenylene, alkenylene-L^{a}-, alkynylene, alkynylene-L^{a}-, -L^{a}-, -L^{a}-alkylene-L^{a}-, -L^{a}-alkenylene-L^{a}-, -L^{a}-alkynylene-L^{a}-, and combinations thereof, wherein each alkylene, alkenylene, and alkynylene is optionally substituted and each occurrence of L^{a} is independently selected from —O—, —S—, —N(R⁷)—, —C(O)—, —C(S)—, —C(O)O—, —OC(O)—, —OC(O)O—, —C(O)N(R⁷)—, —N(R⁷)C(O)—, —C(O)N(R⁷)C(O)—, —C(O)N(R⁷)C(O)N(R⁷), —N(R⁷)C(O)N(R⁷)—, —N(R⁷)C(O)O—, —OC(O)N(R⁷)—, —C(NR⁷)—, —N(R⁷)C(NR⁷)—, —C(NR⁷)N(R⁷)—, —N(R⁷)C(NR⁷)N(R⁷)—, —S(O)₂—, —OS(O)—, —S(O)O—, —S(O), —OS(O)₂—, —S(O)₂O, —N(R⁷)S(O)₂—, —S(O)₂N(R⁷)—, —N(R⁷)S(O)—, —S(O)N(R⁷)—, —N(R⁷)S(O)₂N(R⁷)—, and —N(R⁷)S(O)N(R⁷)— and R⁷ is independently selected at each occurrence from hydrogen, —NH₂, —C(O)OCH₂C₆H₅; and C₁₋₁₀ alkyl, C₂₋₁₀ alkenyl, C₂₋₁₀ alkynyl, C₃₋₁₂ cycloalkyl, and 3- to 12-membered heterocycle, each of which is optionally substituted with one or more substituents independently selected from halo, hydroxyl, cyano, nitro, amino, oxo, thioxo, —C(O)OCH₂C₆H₅, —NHC(O)OCH₂C₆H₅, C₁₋₁₀ alkyl, C₁₋₁₀ haloalkyl, C₁₋₁₀ alkoxy, C₂₋₁₀ alkenyl, C₂₋₁₀ alkynyl, C₃₋₁₂ cycloalkyl, and 3- to 12-membered heterocyclyl.

In some embodiments, each L¹, L², or L³ is optionally substituted with one or more substituents selected from alkyl, alkenyl, alkynyl, halo, hydroxyl, cyano, —OR⁸, —SR⁸, amino, aminyl, amido, cycloalkyl, aryl, heterocyclyl, heteroaryl, cycloclkylalkyl, arylalkyl, heterocyclylalkyl, heteroarylalkyl, —C(O)R⁸, —C(O)N(R⁸)₂, —N(R⁸)C(O)R⁸, —C(O)OR⁸, —OC(O)R⁸, —S(O)R⁸, —S(O)₂R⁸, —P(O)(OR⁸)₂, —OP(O)(OR⁸)₂, nitro, oxo, thioxo, ═N(R⁸), or cyano, and R⁸ is hydrogen, alkyl, alkenyl, alkynyl, cycloalkyl, aryl, heterocyclyl, heteroaryl, cycloalkylalkyl, arylalkyl, heterocyclylalkyl, or heteroarylalkyl.

In some embodiments, L¹, L², or L³ are independently selected from the following structures:

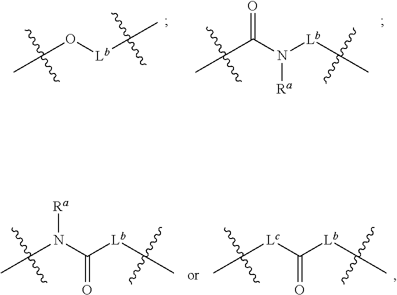

wherein:
R^{a} is hydrogen or alkyl;
each occurrence of L^{b} is independently a direct bond, an optionally substituted alkylene linker, an optionally substituted heteroalkylene linker, a heteroatomic linker, or a combination thereof; and each occurrence of L^{c} is independently an optionally substituted alkylene linker and provided that at least one of L¹, L², or L³ has the following structure:

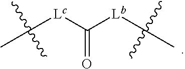

In some embodiments, L¹ and L² are independently selected from the following structures:

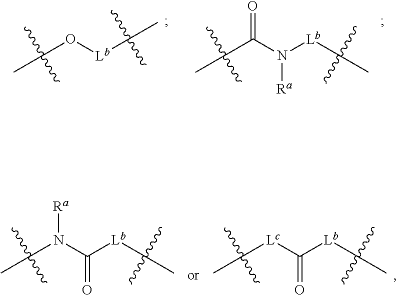

wherein:
R^{a} is hydrogen or alkyl;
each occurrence of L^{b} is independently a direct bond, an optionally substituted alkylene linker, an optionally substituted heteroalkylene linker, a heteroatomic linker, or a combination thereof;
each occurrence of L^{c} is independently an optionally substituted alkylene linker; provided that at least one of L¹ or L² has the following structure:

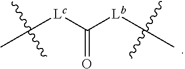

In more embodiments, L² has the following structure:

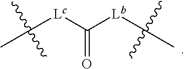

In some embodiments, L^{c} is unsubstituted. In some embodiments, L^{c} is a C₁-C₆ alkylene. In some more embodiments, L^{c} is a C₂-C₄ alkylene. In some embodiments, L^{c} is a straight C₁-C₆ alkylene. In more embodiments, L^{c} is a straight, unsubstituted C₁-C₆ alkylene. In more embodiments, L^{c} is a straight, unsubstituted C₂-C₄ alkylene.

In more embodiments, L¹, L², and L³ each independently have one of the following structures:

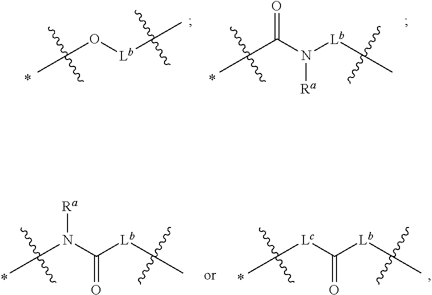

wherein:
* indicates a direct bond to a substitutable position on the phenyl group of following structure:

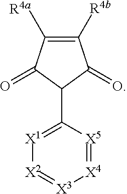

In some embodiments, L¹ or L² has one of the following structures:

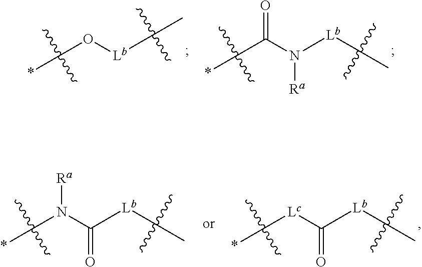

wherein:
* indicates a direct bond to a substitutable position on the phenyl group of following structure:

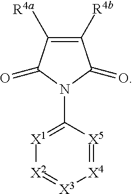

In some embodiments, L^{c} is substituted with one or more substituents selected from the group consisting of halo, haloalkyl, alkoxy, cyano, nitro, carboxy, sulfonamide, sulfonic acid, or combinations thereof.

In some embodiments, the compound has one of the following Structures (Ia) or (Ib):

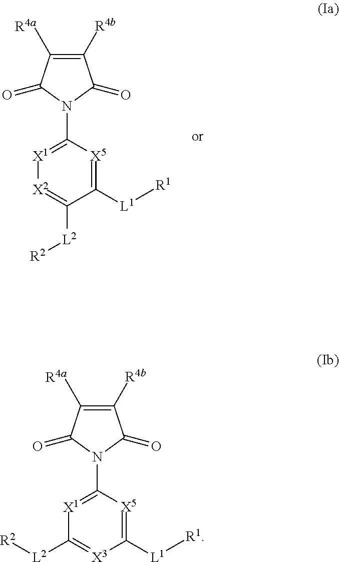

In some embodiments, the compound has one of the following Structures (Ic′) or (Ic″):

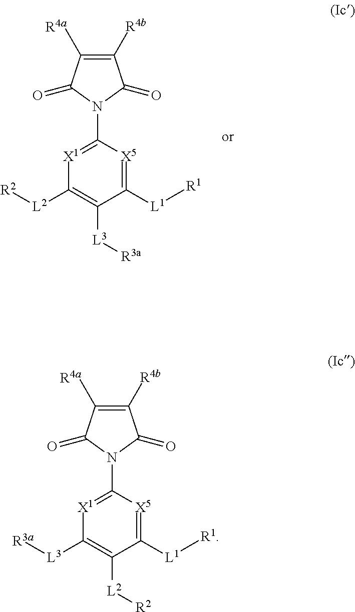

In certain embodiments, X², X³, or both are C—H, or C—F. In some embodiments, X¹, X⁵, or both are C—R³ and R³ is H or halo. In some embodiments, X¹ and X⁵ are both C—R³ and R³ is H or halo. In some embodiments, X¹ is C—R³ and R³ is halo. In certain embodiments, X⁵ is C—R³ and R³ is halo. In some embodiments, halo is fluoro. In some embodiments, X¹ is C—F. In some embodiments, X¹ is C—H. In some embodiments, X⁵ is C—H. In some embodiments, X⁵ is C—F. In some embodiments, X³ is C—R³ and R³ is H. In some embodiments, X³ is C—R³ and R³ is halo (e.g., fluoro).

In some embodiments, the compound has one of the following structures (Ia-1), (Ia-2), (Ia-3), (Ia-4), (Ia-5), or (Ia-6):

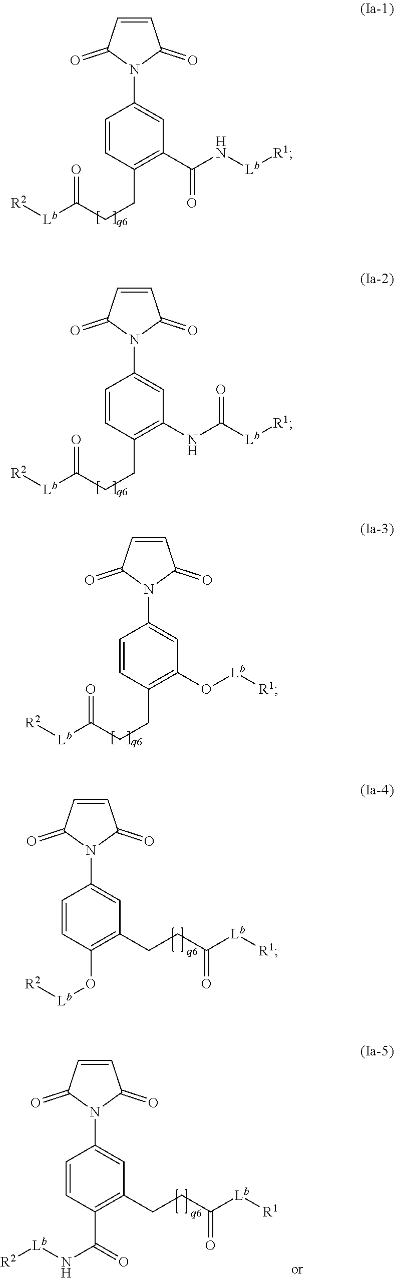

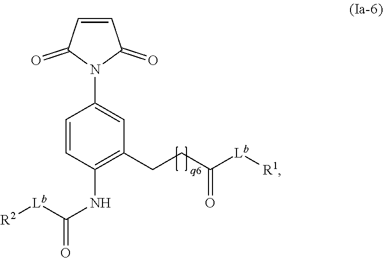

wherein:
each occurrence of L^{b} is independently a direct bond, an optionally substituted alkylene linker, an optionally substituted heteroalkylene linker, a heteroatomic linker, or combinations thereof; and
q6 is 0, 1, or 2.

In some embodiments, the compound has one of the following structures (Ia-1), (Ia-2), (Ia-3), (Ia-4), (Ia-5), (Ia-6), (Ia-7), or (Ia-8):

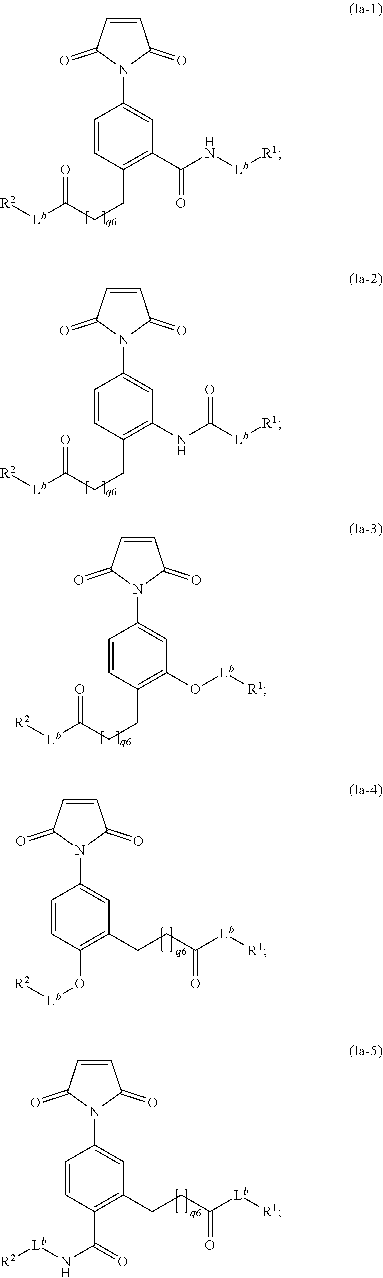

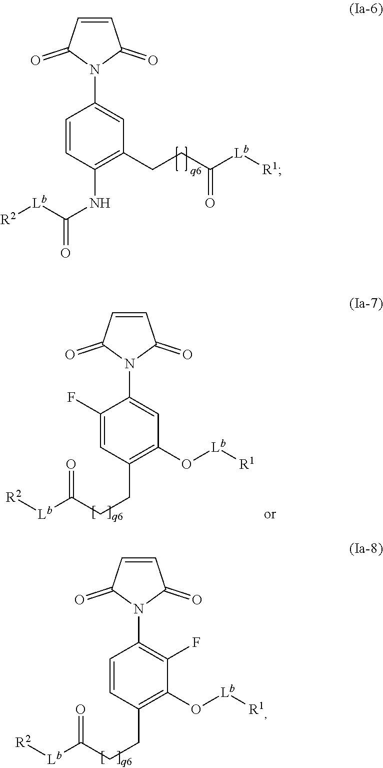

wherein:
each occurrence of L^{b} is independently a direct bond, an optionally substituted alkylene linker, an optionally substituted heteroalkylene linker, a heteroatomic linker, or combinations thereof; and
q6 is 0, 1, or 2.

In some embodiments, q6 is 1 and L^{b} is gly-gly.

In certain embodiments, the compound has one of the following Structures (Ic-1), (Ic-2), (Ic-3), or (Ic-4):

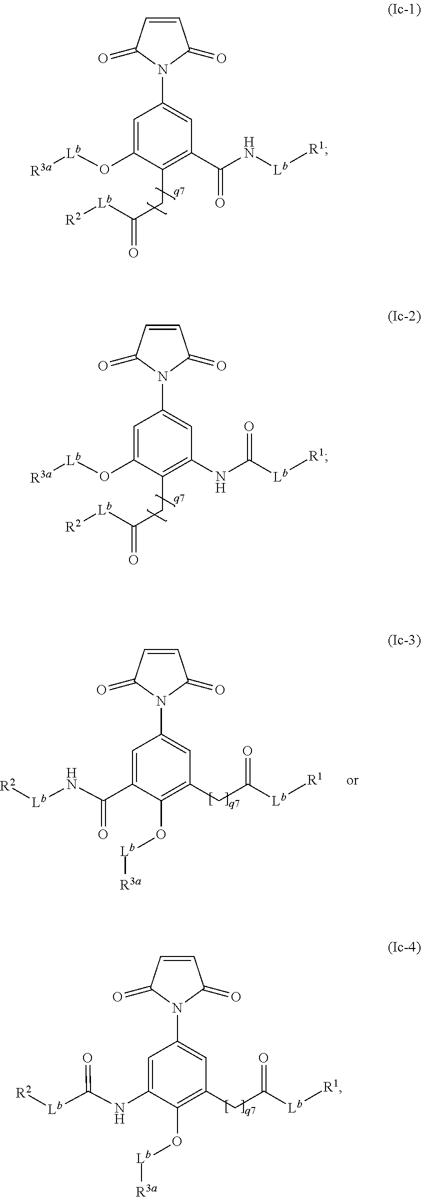

wherein:
each occurrence of L^{b} is independently a direct bond, an optionally substituted alkylene linker, an optionally substituted heteroalkylene linker, a heteroatomic linker, or combinations thereof; and
q7 is 1, 2, or 3.

In some embodiments, q7 is 2.

In some embodiments, the compound has the following Structure (Id), (Ie), (If), or (Ig):

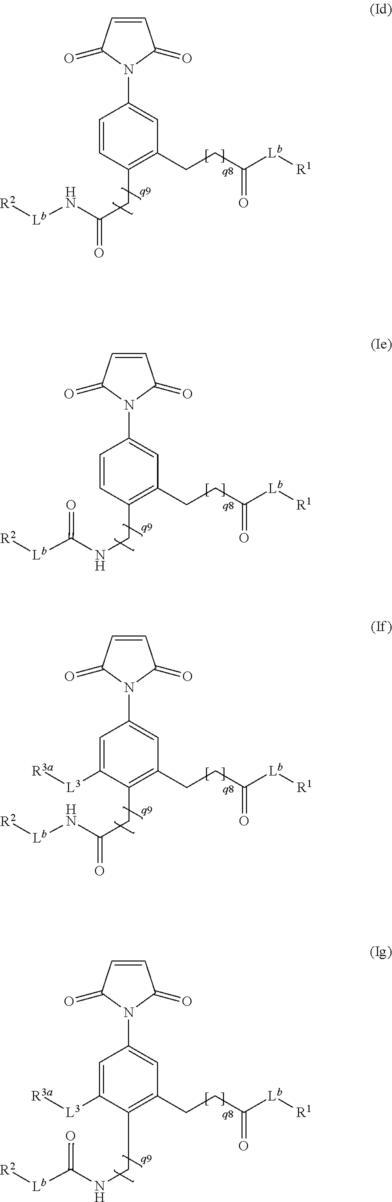

wherein:
each occurrence of L^{b} is independently a direct bond, an optionally substituted alkylene linker, an optionally substituted heteroalkylene linker, a heteroatomic linker, or combinations thereof;
q8 is 0, 1, or 2; and
q9 is 0, 1, or 2.

In some embodiments, q9 is 0 and q8 is 1.

In some embodiments, the compound has one of the following Structures (Ih) or (Ii):

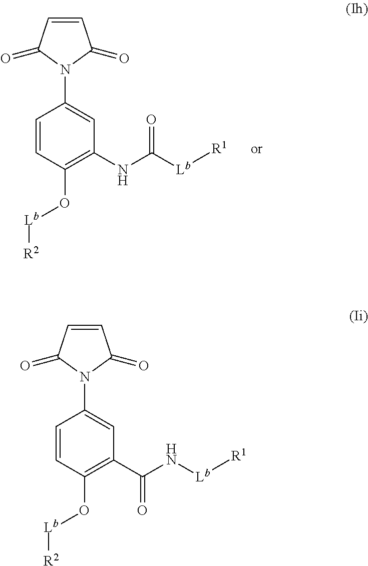

wherein:
each occurrence of L^{b} is independently a direct bond, an optionally substituted alkylene linker, an optionally substituted heteroalkylene linker, a heteroatomic linker, or combinations thereof.

In some embodiments, L^{b} is a direct bond, an optionally substituted alkylene linker or an optionally substituted heteroalkylene linker.

In some embodiments, L^{b} is a direct bond or has one of the following structures:

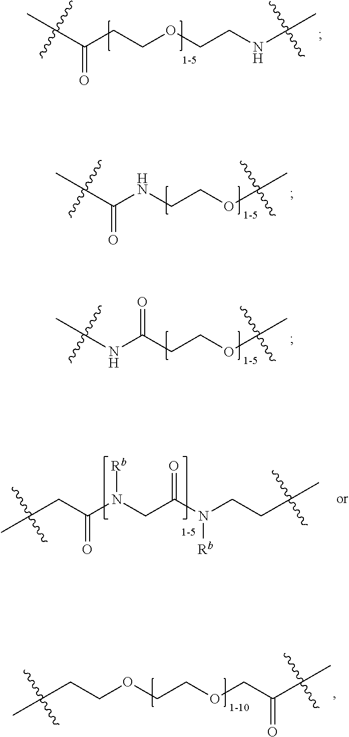

wherein:
each occurrence of R^{b} is independently hydrogen, alkyl, hydroxyalkyl, or alkoxyalkyl.

In some embodiments, each occurrence of R^{b} is —CH₃.

In some embodiments, L¹ or L² has the following structure:

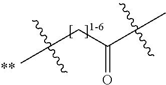

wherein:
** shows a bond to X¹, X², X³, X⁴ or X⁵.

In certain embodiments, X² is C-L¹-R¹ and X³ is C-L²-R². In some embodiments, X³ is C-L¹-R¹ and X² is C-L²-R². In some embodiments, X¹, X⁴, and X⁵ are all CR³. In some embodiments, X¹, X⁴, and X⁵ are all CH.

In some embodiments, the compound has one of the following structures:

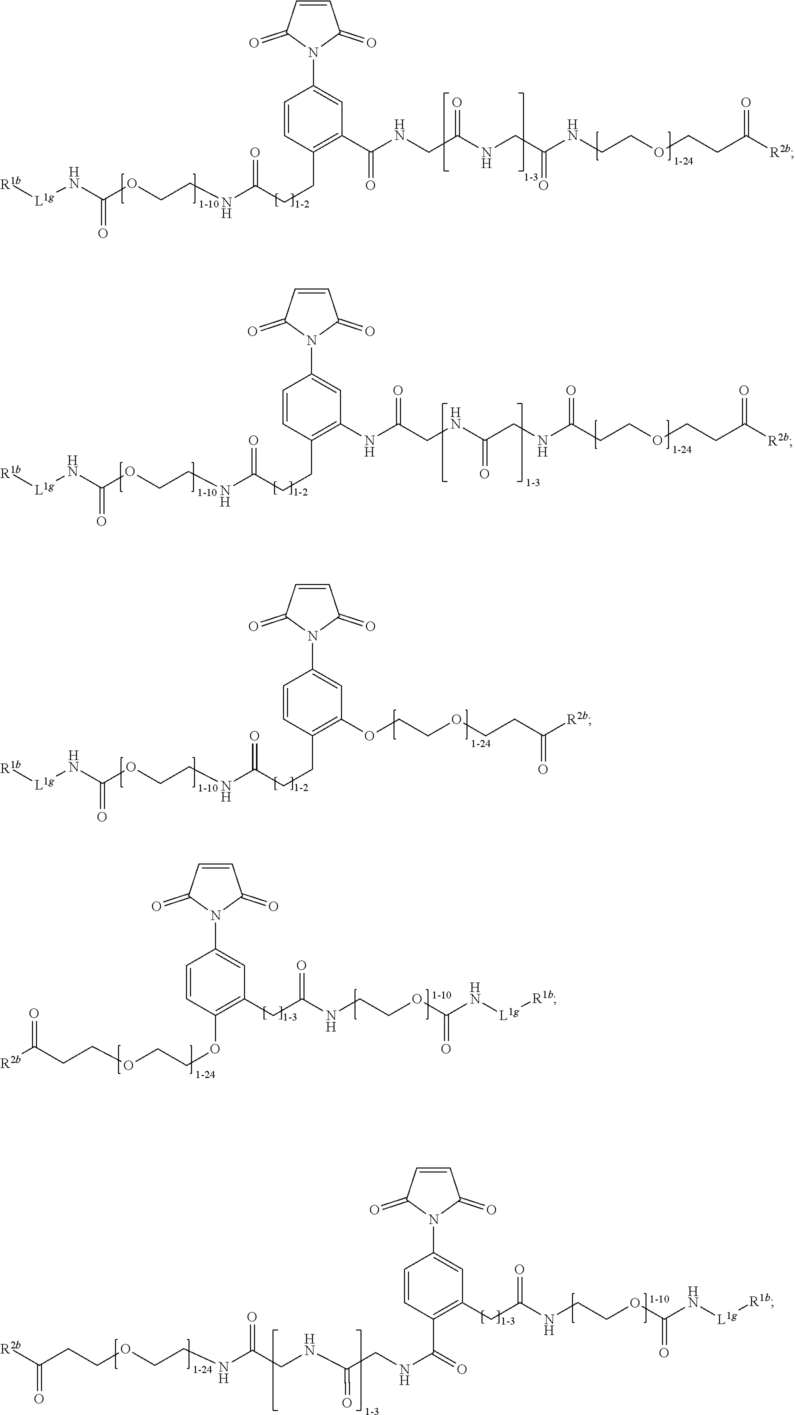

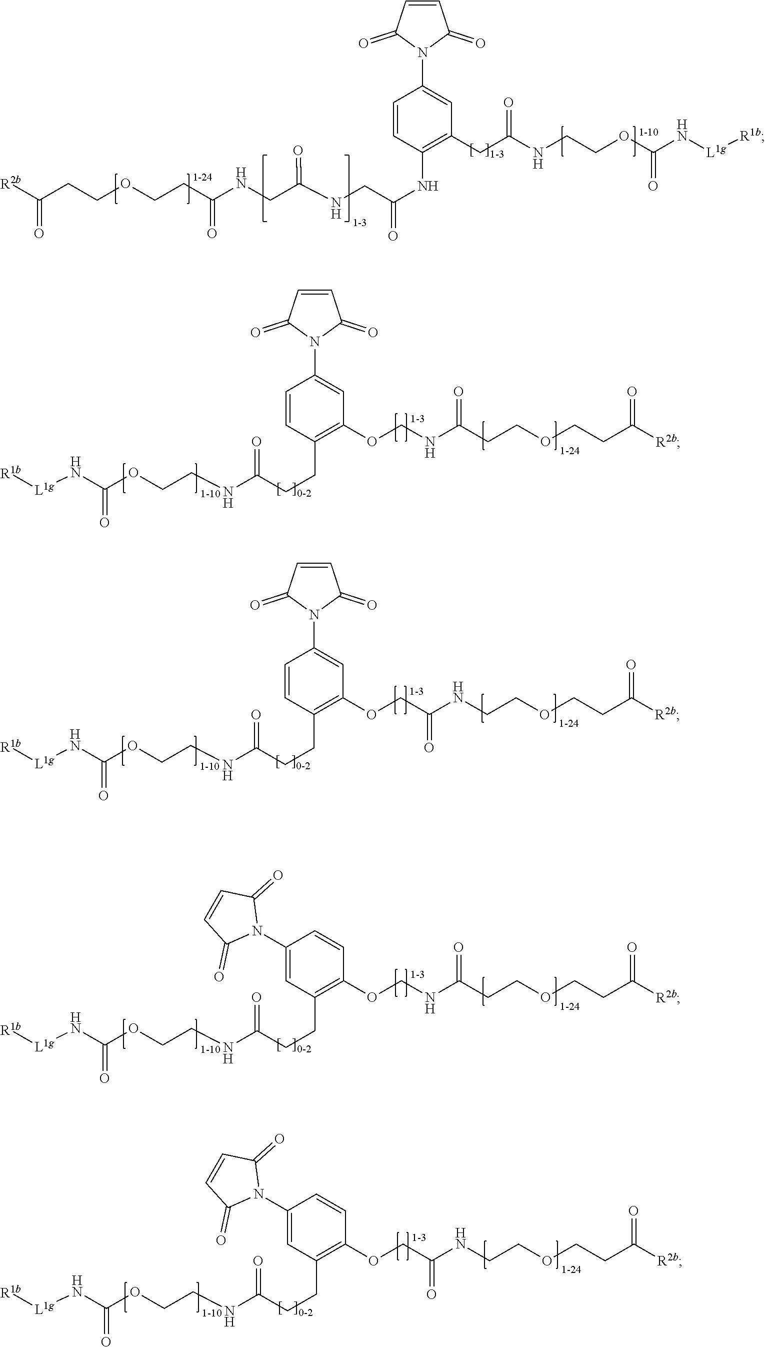

wherein:
R^{1b} is a chemotherapeutic, a cytotoxic agent, or a myeloid cell agonist;
R^{2b} has one of the following structures:

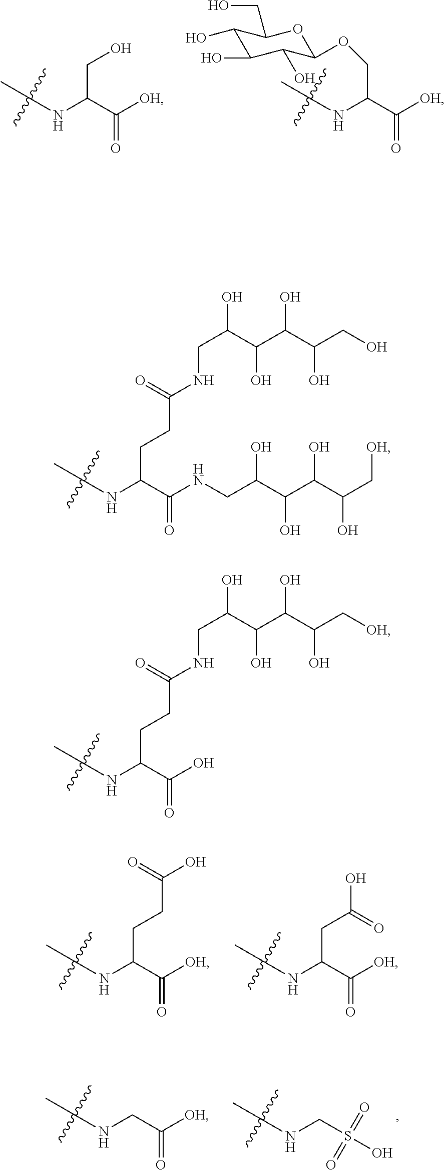

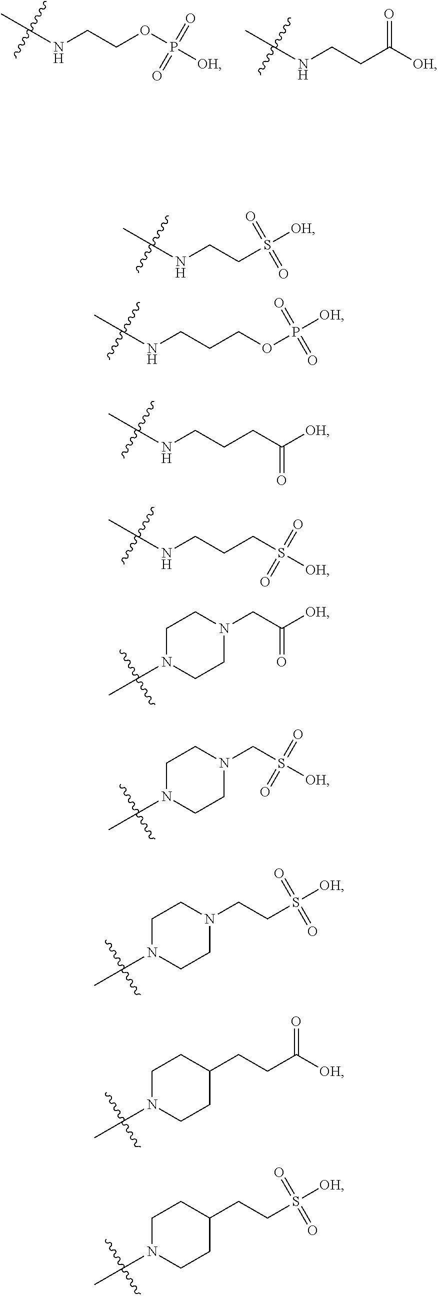

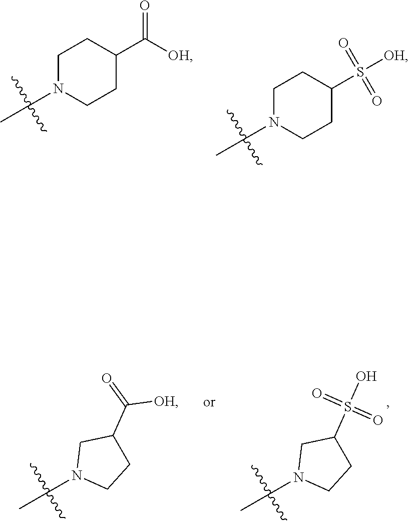 [SMILES: *N1CCC(C(=O)O)CC1 ; *N1CCC(S(=O)(=O)O)CC1 ; *N1CCC(C(=O)O)C1 ; *N1CCC(S(=O)(=O)O)C1]

L^{1g} has one of the following structures:

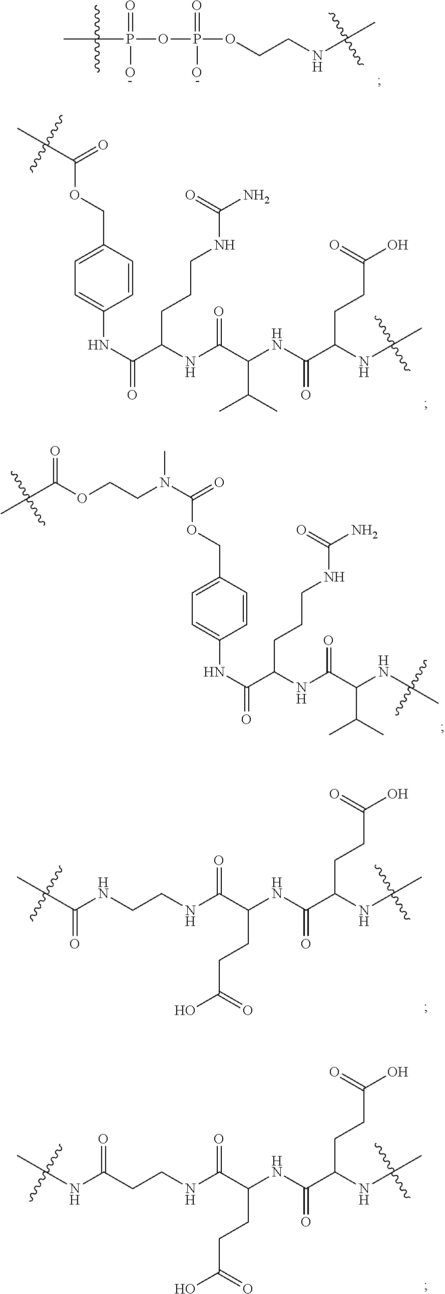 [SMILES: *NCCOP(=O)(O)OP(*)(=O)O ; *NC(CCC(=O)O)C(=O)NC(C(=O)NC(CCCNC(N)=O)C(=O)Nc1ccc(COC(*)=O)cc1)C(C)C ; *NC(C(=O)NC(CCCNC(N)=O)C(=O)Nc1ccc(COC(=O)N(C)CCOC(*)=O)cc1)C(C)C ; *NC(CCC(=O)O)C(=O)NC(CCC(=O)O)C(=O)NCCNC(*)=O ; *NC(=O)CCNC(=O)C(CCC(=O)O)NC(=O)C(CCC(=O)O)N*]

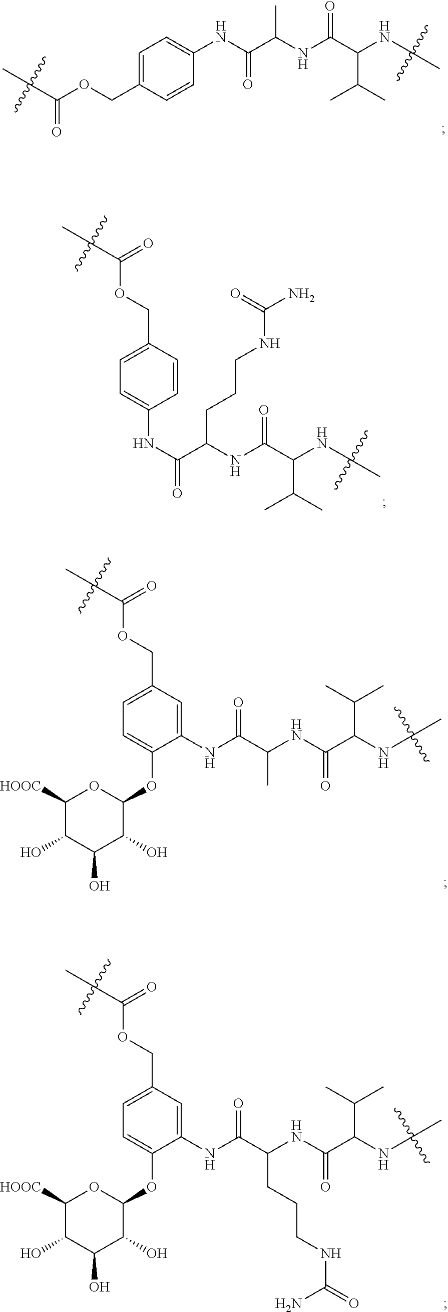 [SMILES: *NC(C(=O)NC(C)C(=O)Nc1ccc(COC(*)=O)cc1)C(C)C ; *NC(C(=O)NC(CCCNC(N)=O)C(=O)Nc1ccc(COC(*)=O)cc1)C(C)C ; *NC(C(=O)NC(C)C(=O)Nc1cc(COC(*)=O)ccc1O[C@@H]1O[C@H](C(=O)O)[C@@H](O)[C@H](O)[C@H]1O)C(C)C ; *NC(C(=O)NC(CCCNC(N)=O)C(=O)Nc1cc(COC(*)=O)ccc1O[C@@H]1O[C@H](C(=O)O)[C@@H](O)[C@H](O)[C@H]1O)C(C)C]

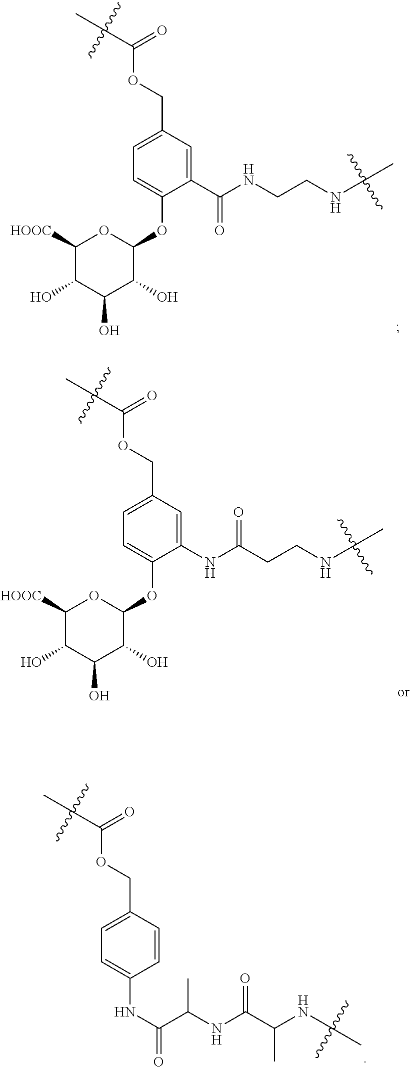 [SMILES: *NCCNC(=O)c1cc(COC(*)=O)ccc1O[C@@H]1O[C@H](C(=O)O)[C@@H](O)[C@H](O)[C@H]1O ; *NCCC(=O)Nc1cc(COC(*)=O)ccc1O[C@@H]1O[C@H](C(=O)O)[C@@H](O)[C@H](O)[C@H]1O ; *NC(C)C(=O)NC(C)C(=O)Nc1ccc(COC(*)=O)cc1]

In some embodiments, the compound has one of the following structures:

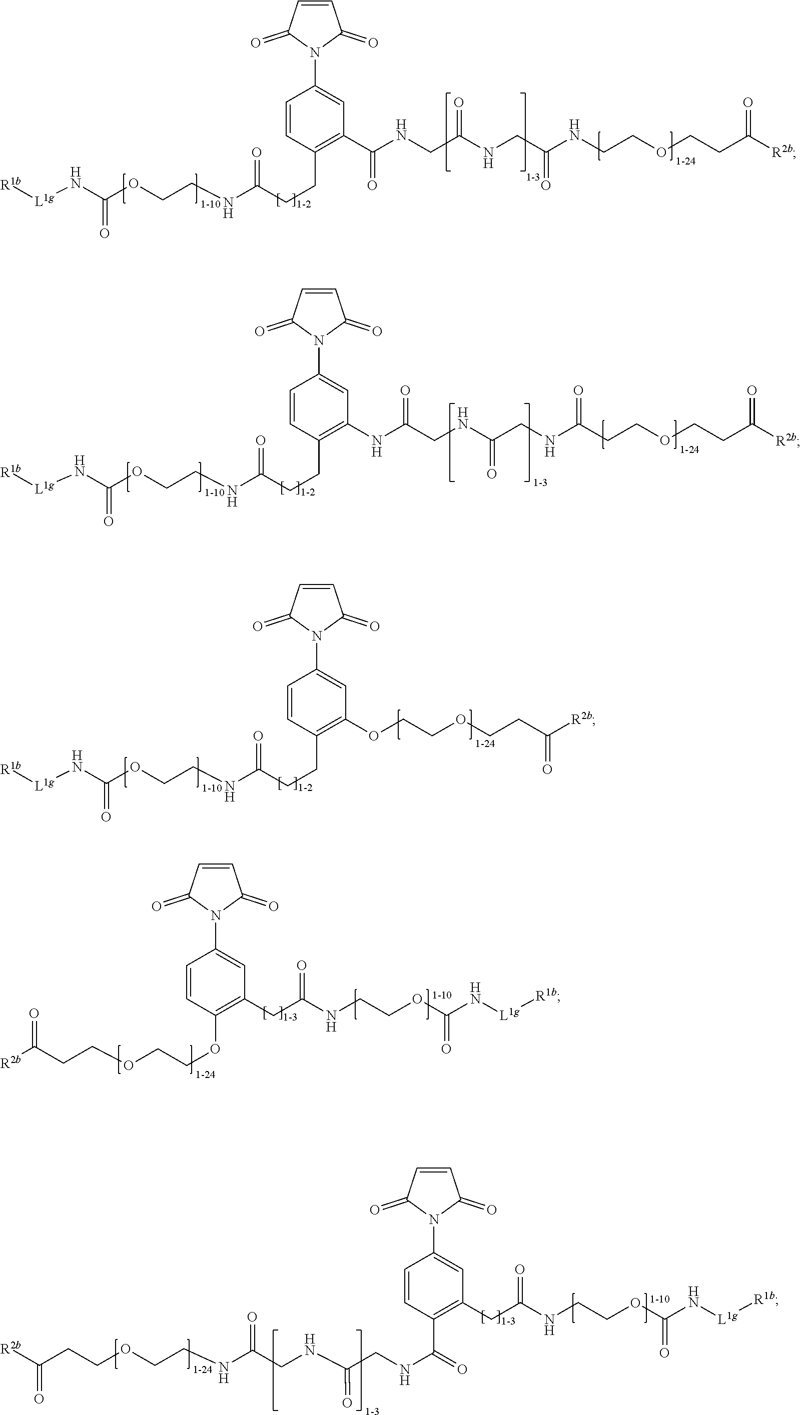

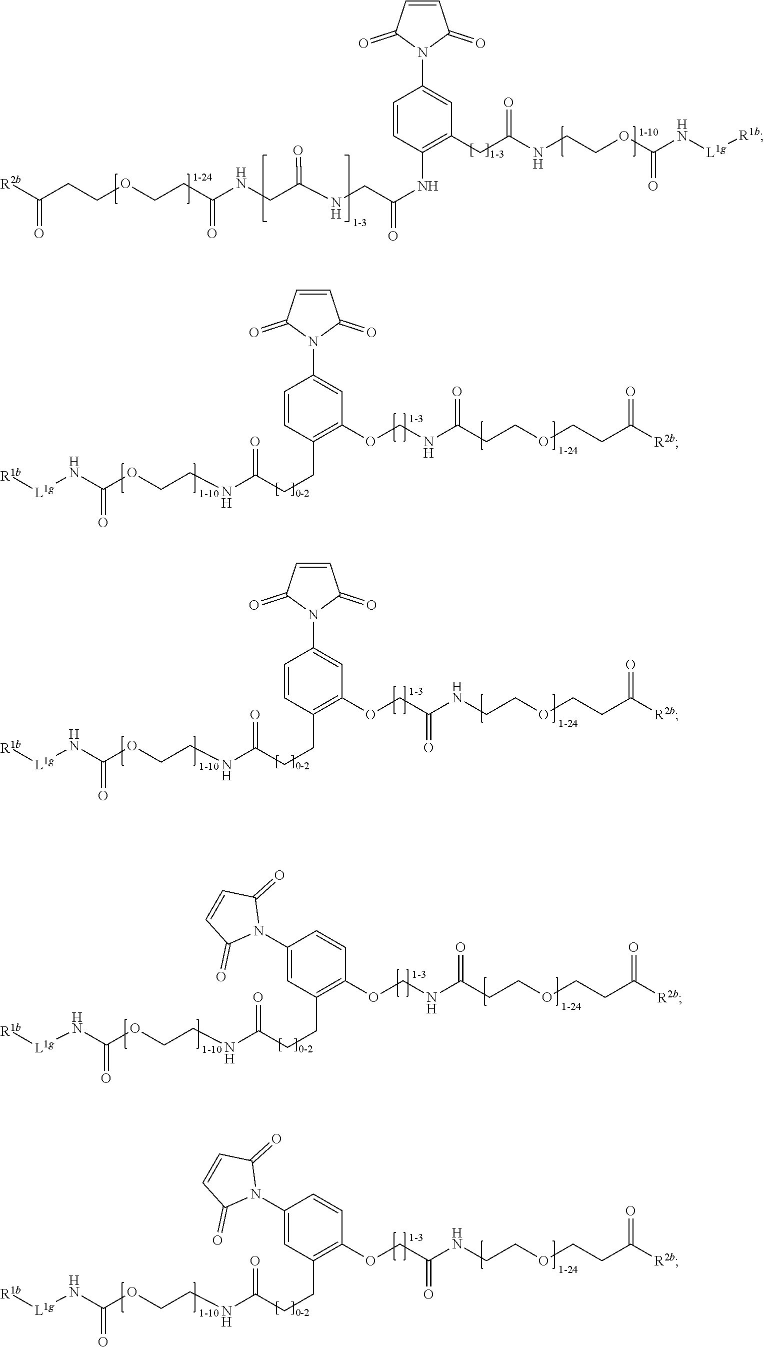

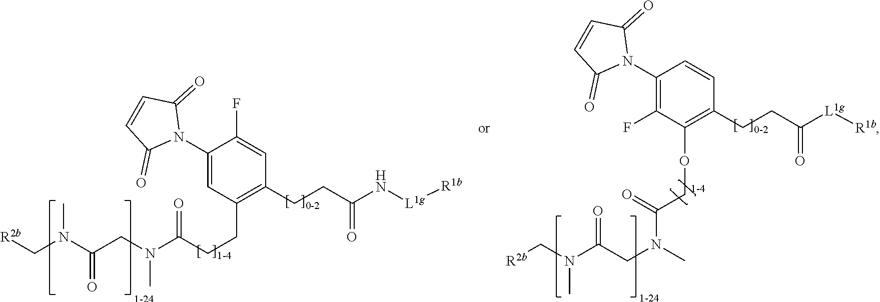

wherein:
R^{1b} is a chemotherapeutic, a cytotoxic agent, or a myeloid cell agonist;
R^{2b} has one of the following structures:

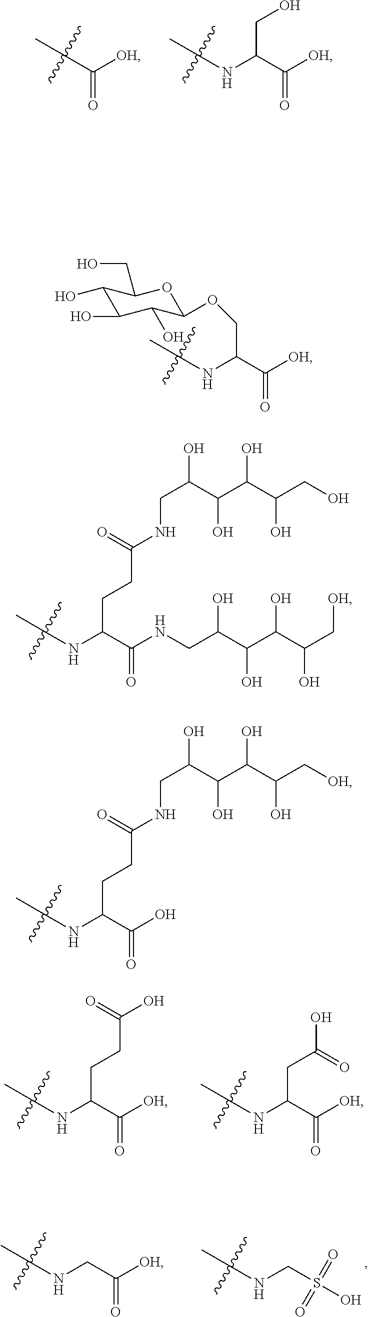

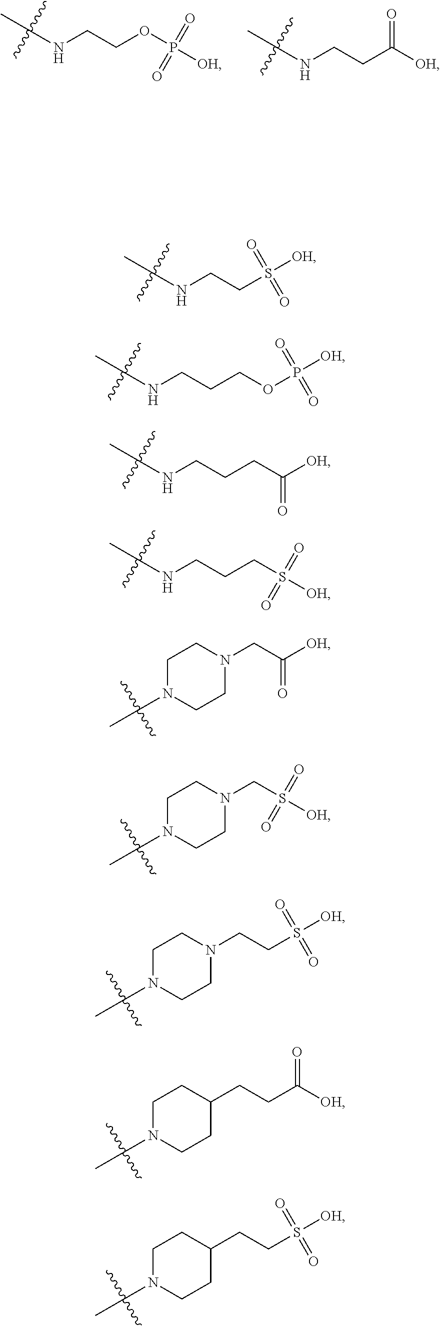

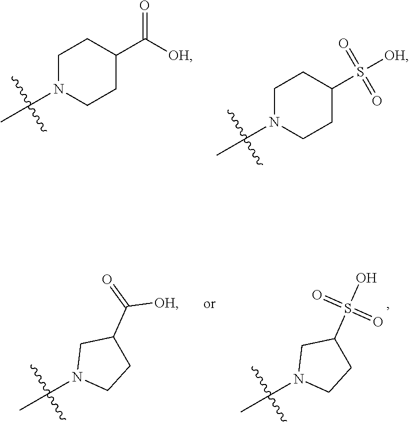 [SMILES: *N1CCC(C(=O)O)CC1 ; *N1CCC(S(=O)(=O)O)CC1 ; *N1CCC(C(=O)O)C1 ; *N1CCC(S(=O)(=O)O)C1]

L^{1g} has one of the following structures:

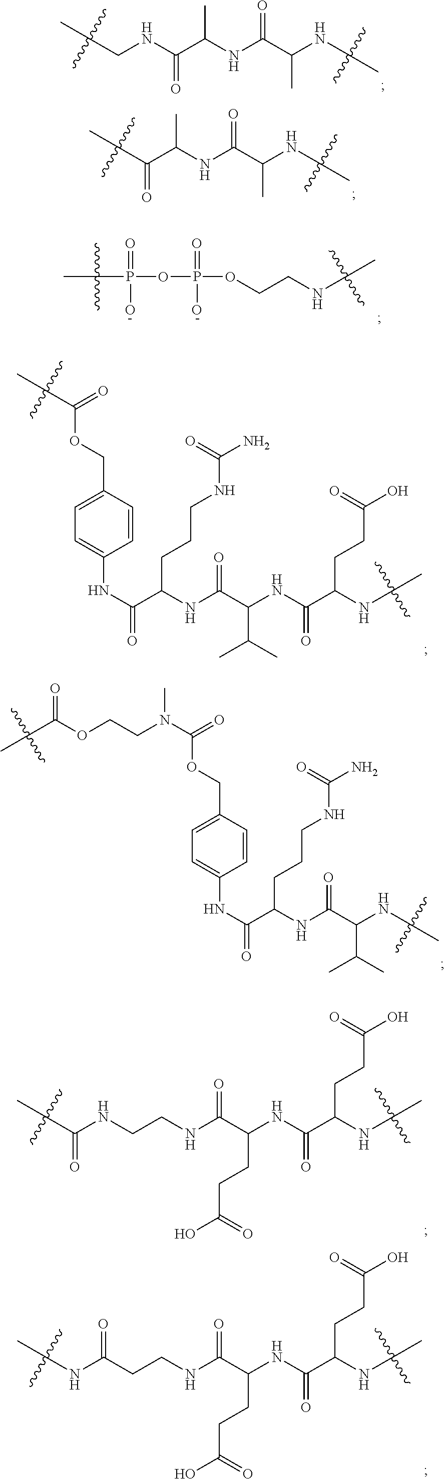

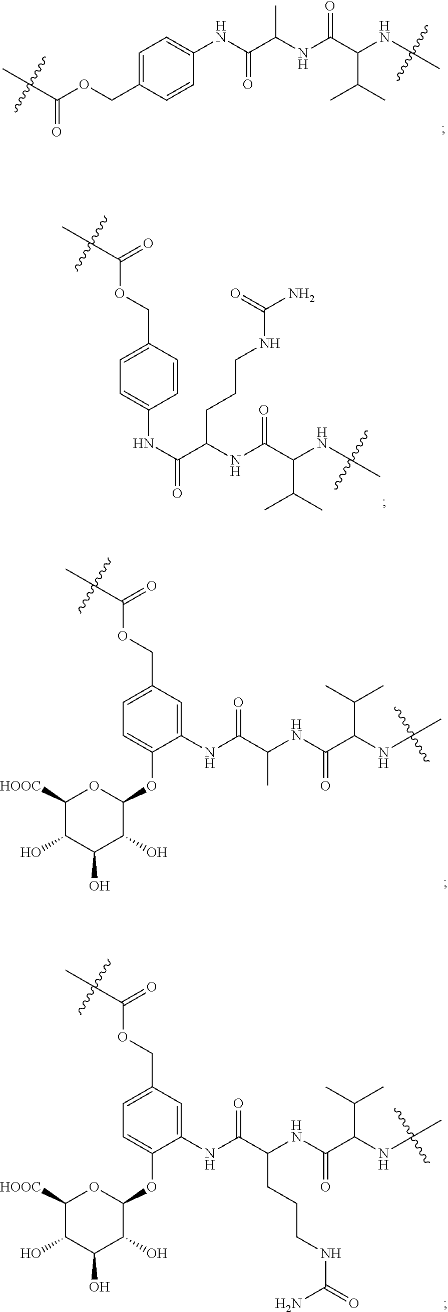 [SMILES: *NC(C(=O)NC(C)C(=O)Nc1ccc(COC(*)=O)cc1)C(C)C ; *NC(C(=O)NC(CCCNC(N)=O)C(=O)Nc1ccc(COC(*)=O)cc1)C(C)C ; *NC(C(=O)NC(C)C(=O)Nc1cc(COC(*)=O)ccc1O[C@@H]1O[C@H](C(=O)O)[C@@H](O)[C@H](O)[C@H]1O)C(C)C ; *NC(C(=O)NC(CCCNC(N)=O)C(=O)Nc1cc(COC(*)=O)ccc1O[C@@H]1O[C@H](C(=O)O)[C@@H](O)[C@H](O)[C@H]1O)C(C)C]

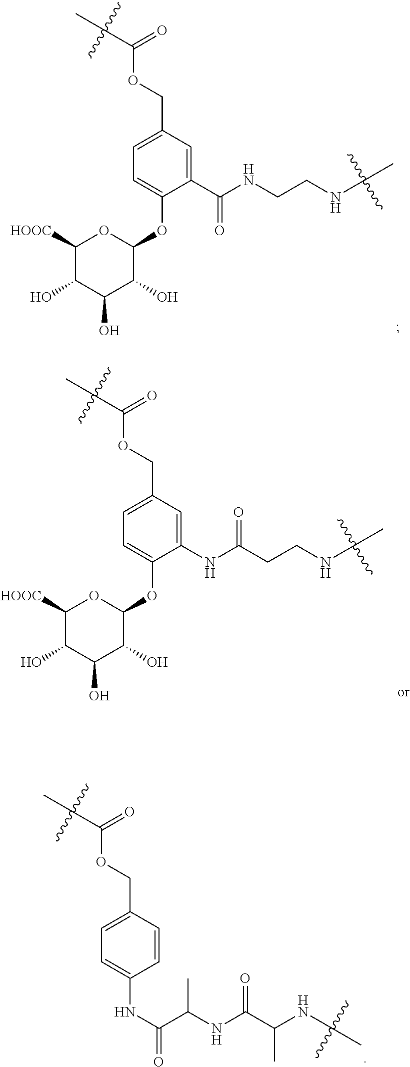 [SMILES: *NCCNC(=O)c1cc(COC(*)=O)ccc1O[C@@H]1O[C@H](C(=O)O)[C@@H](O)[C@H](O)[C@H]1O ; *NCCC(=O)Nc1cc(COC(*)=O)ccc1O[C@@H]1O[C@H](C(=O)O)[C@@H](O)[C@H](O)[C@H]1O ; *NC(C)C(=O)NC(C)C(=O)Nc1ccc(COC(*)=O)cc1]

In some embodiments, the compound has one of the following structures:

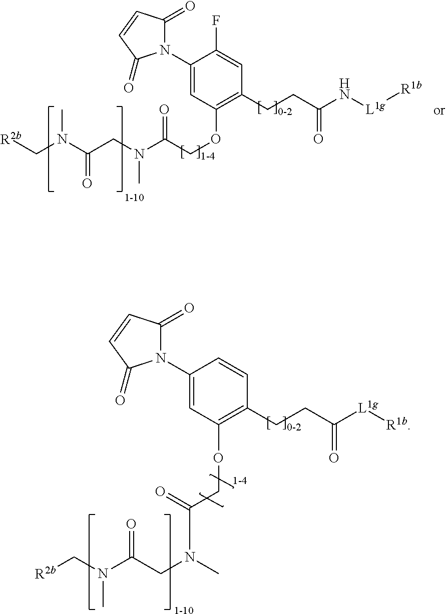

In some embodiments, the compound has one of the following structures:

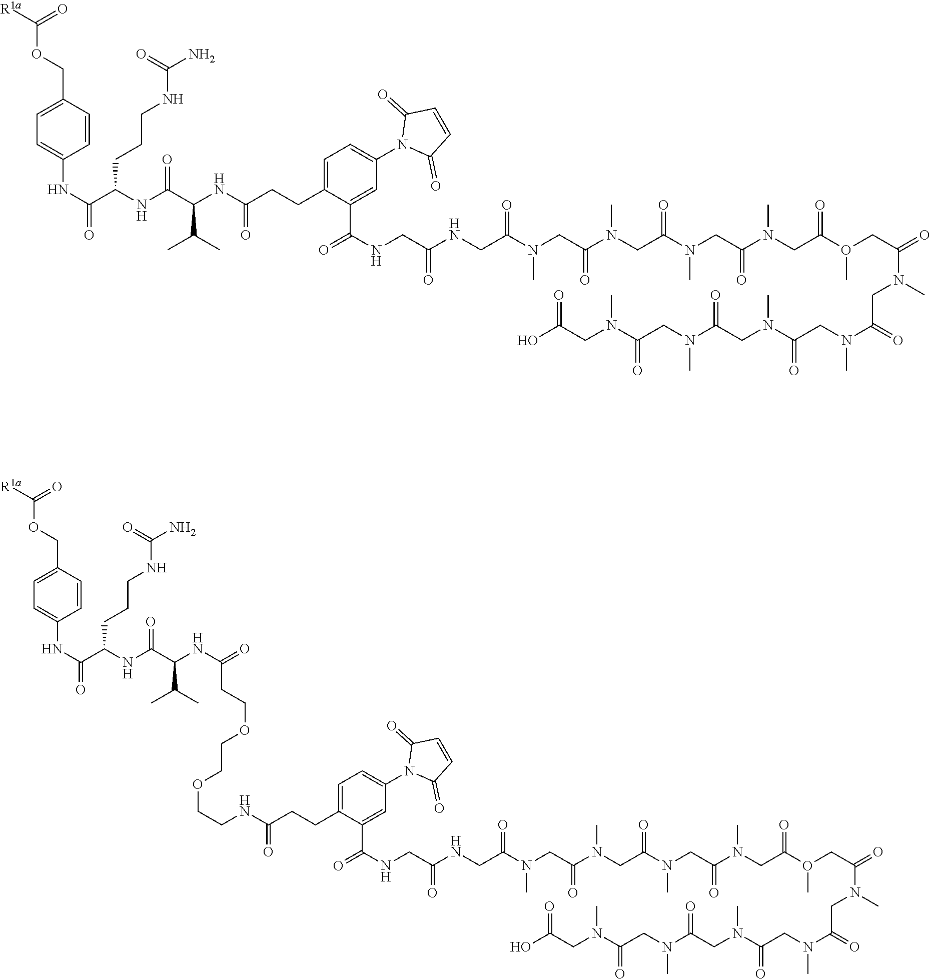

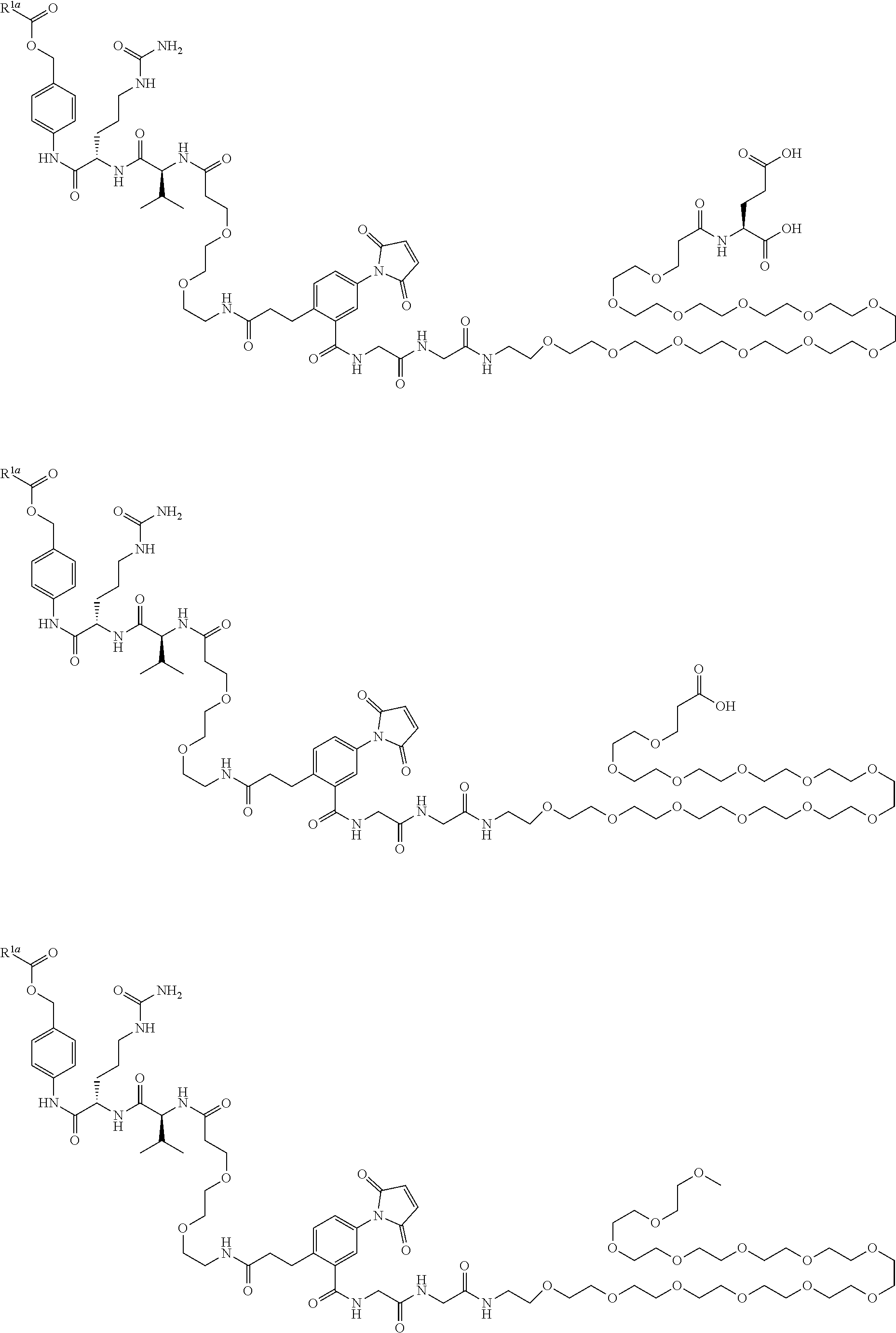

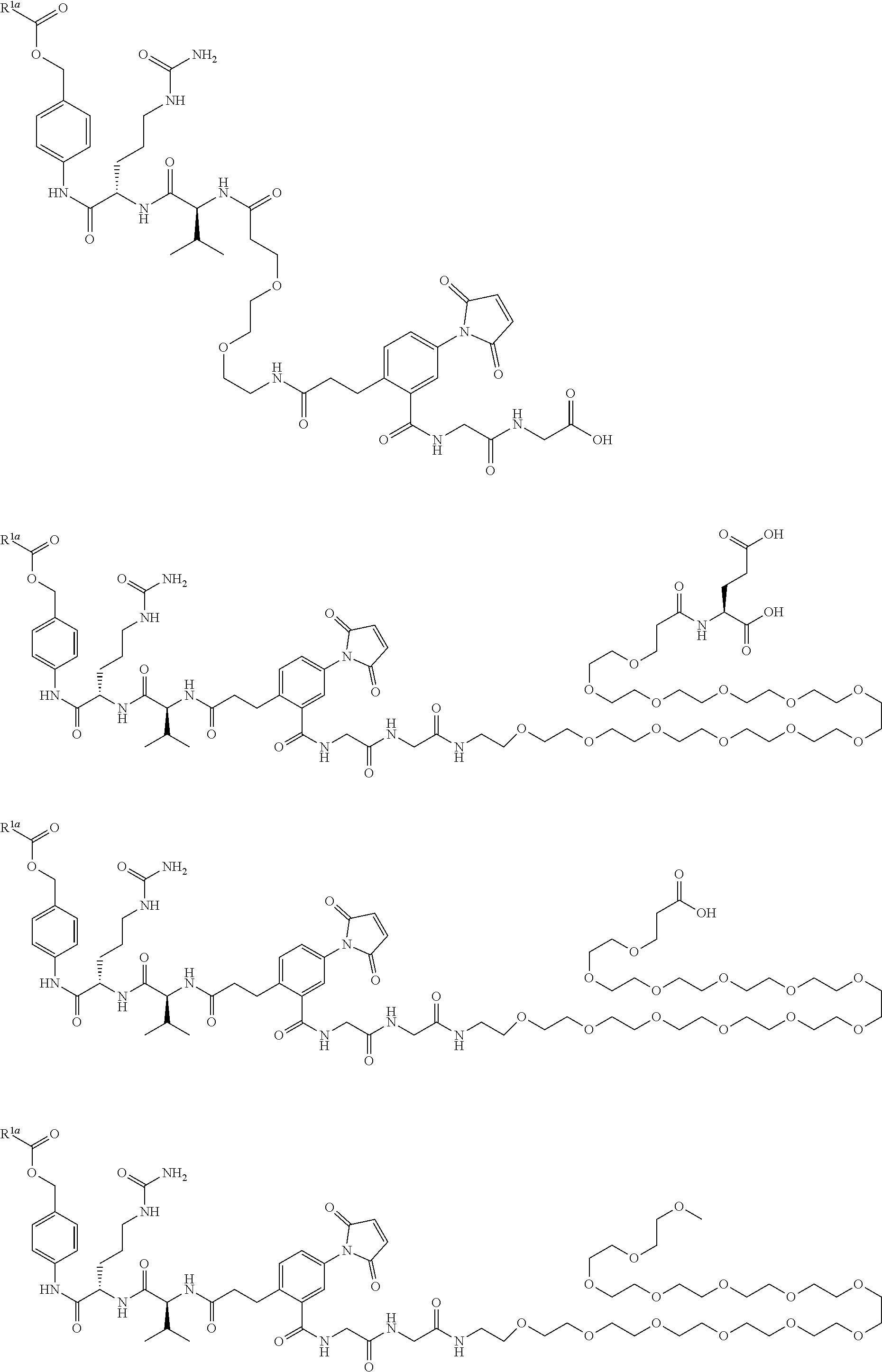

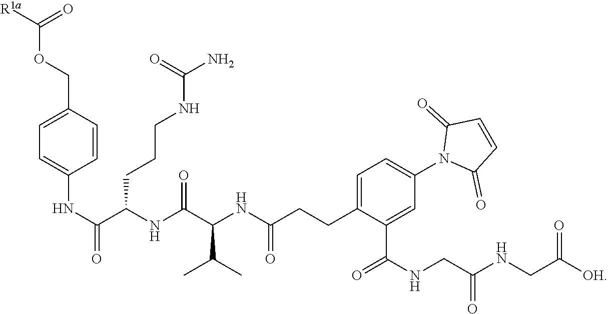

In some embodiments, the compound has one of the following structures:

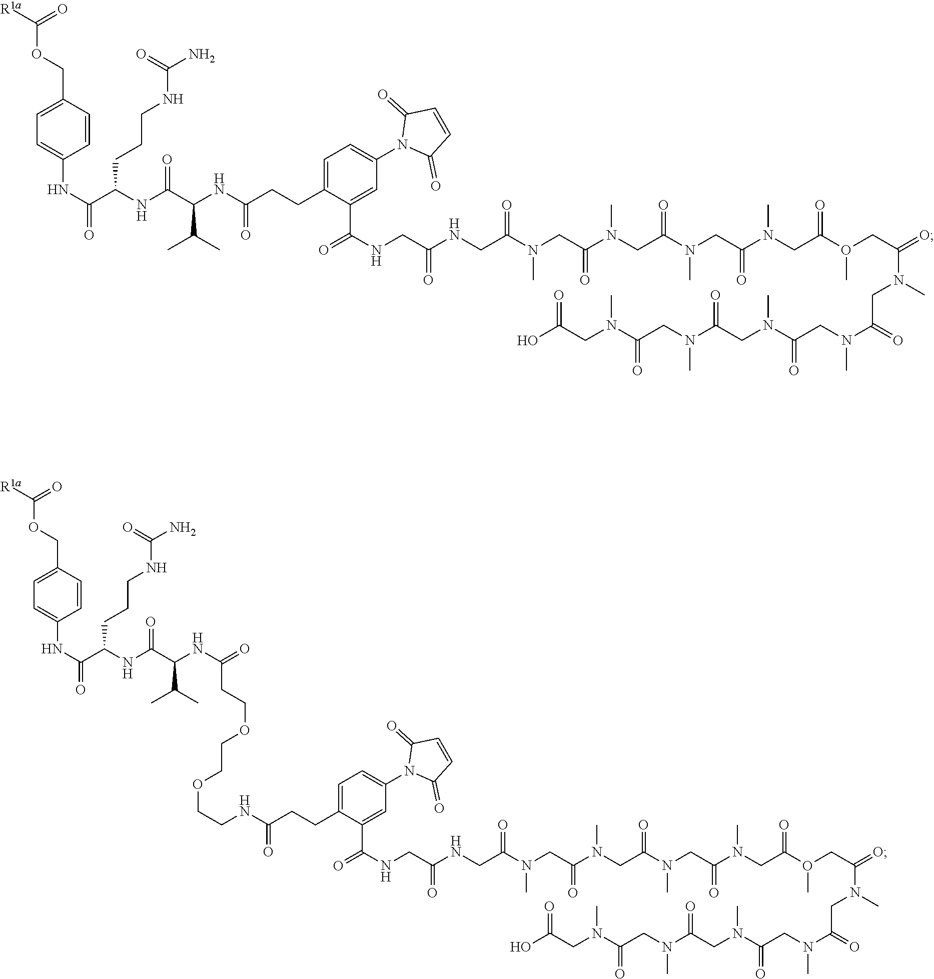

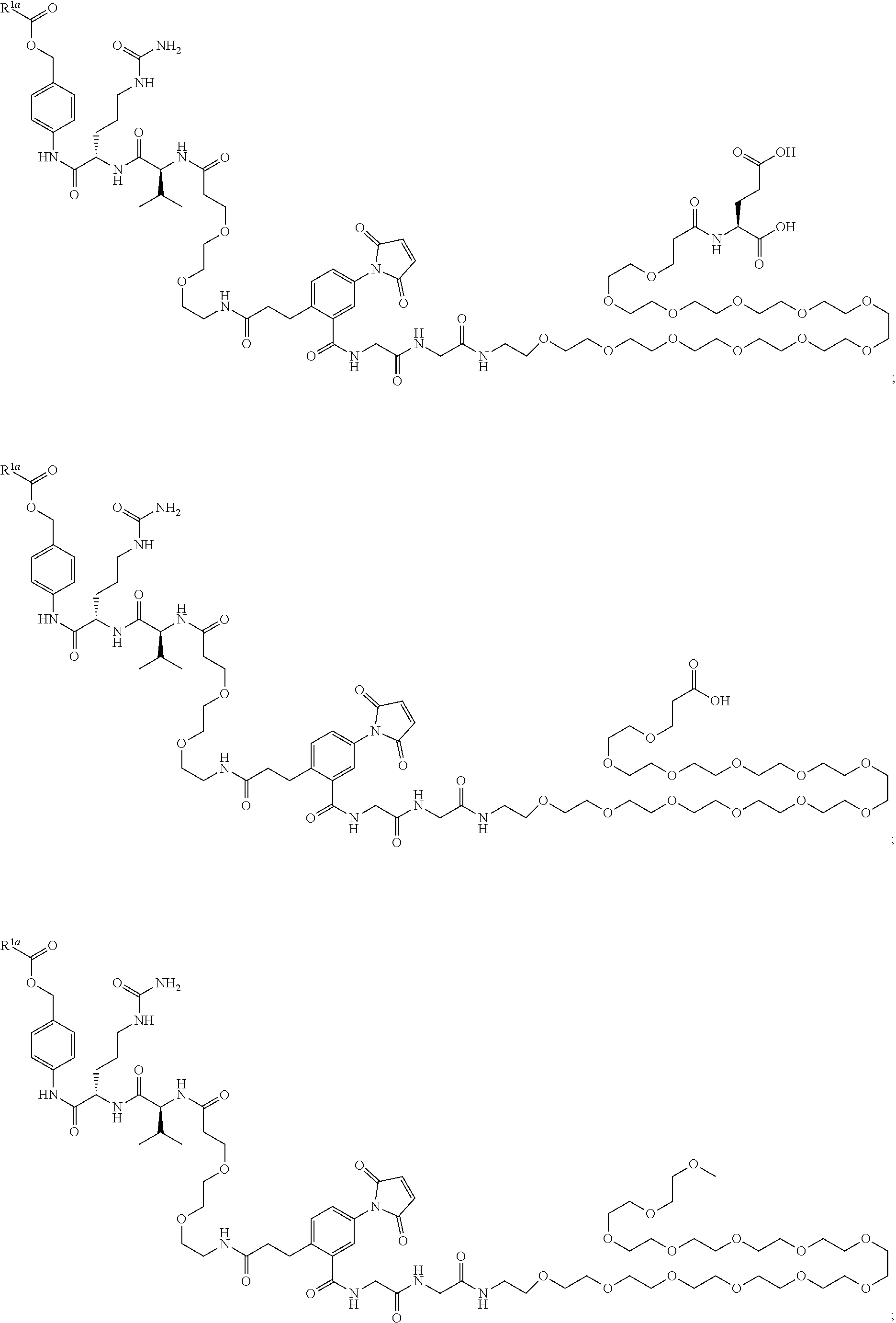

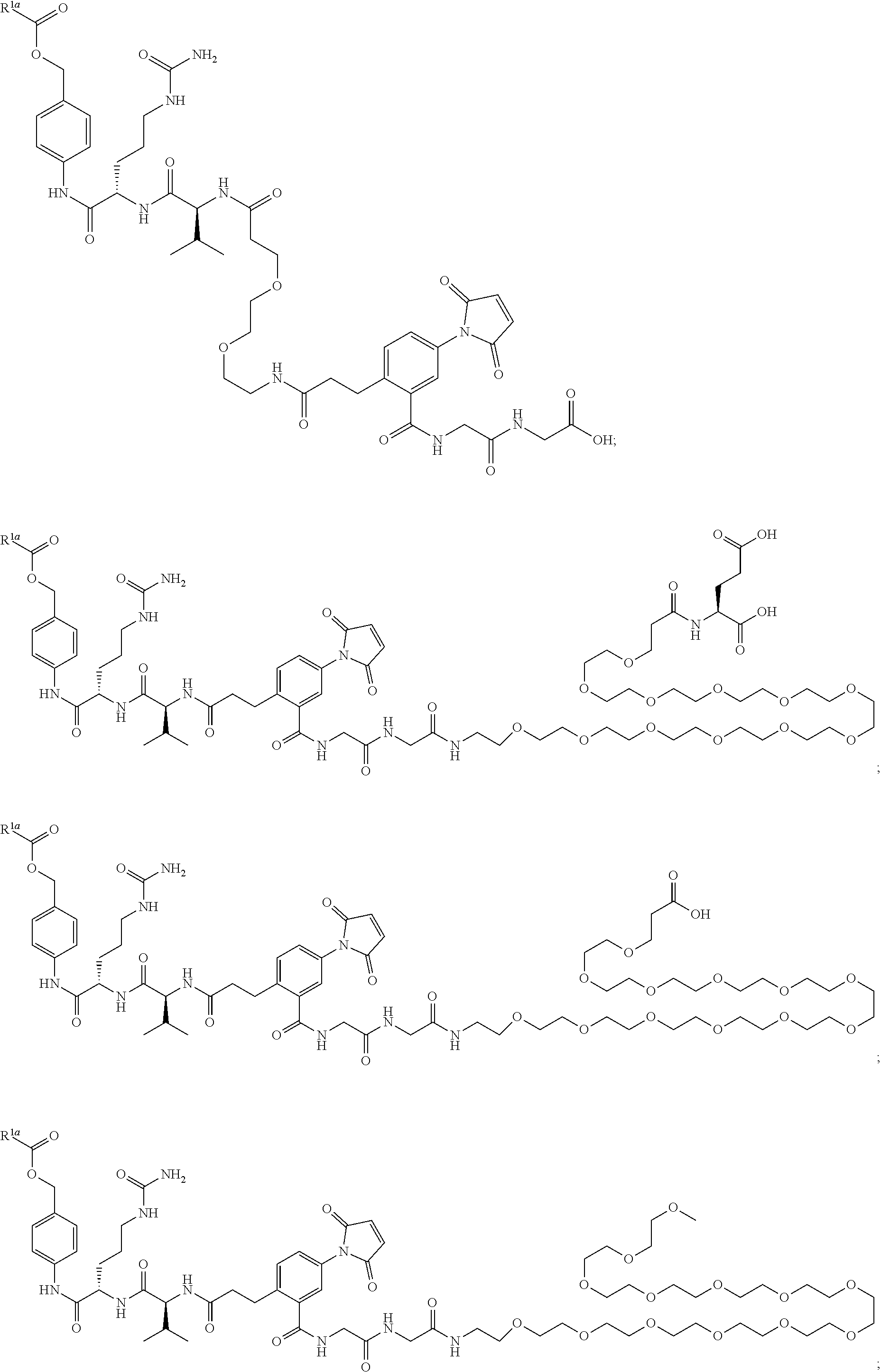

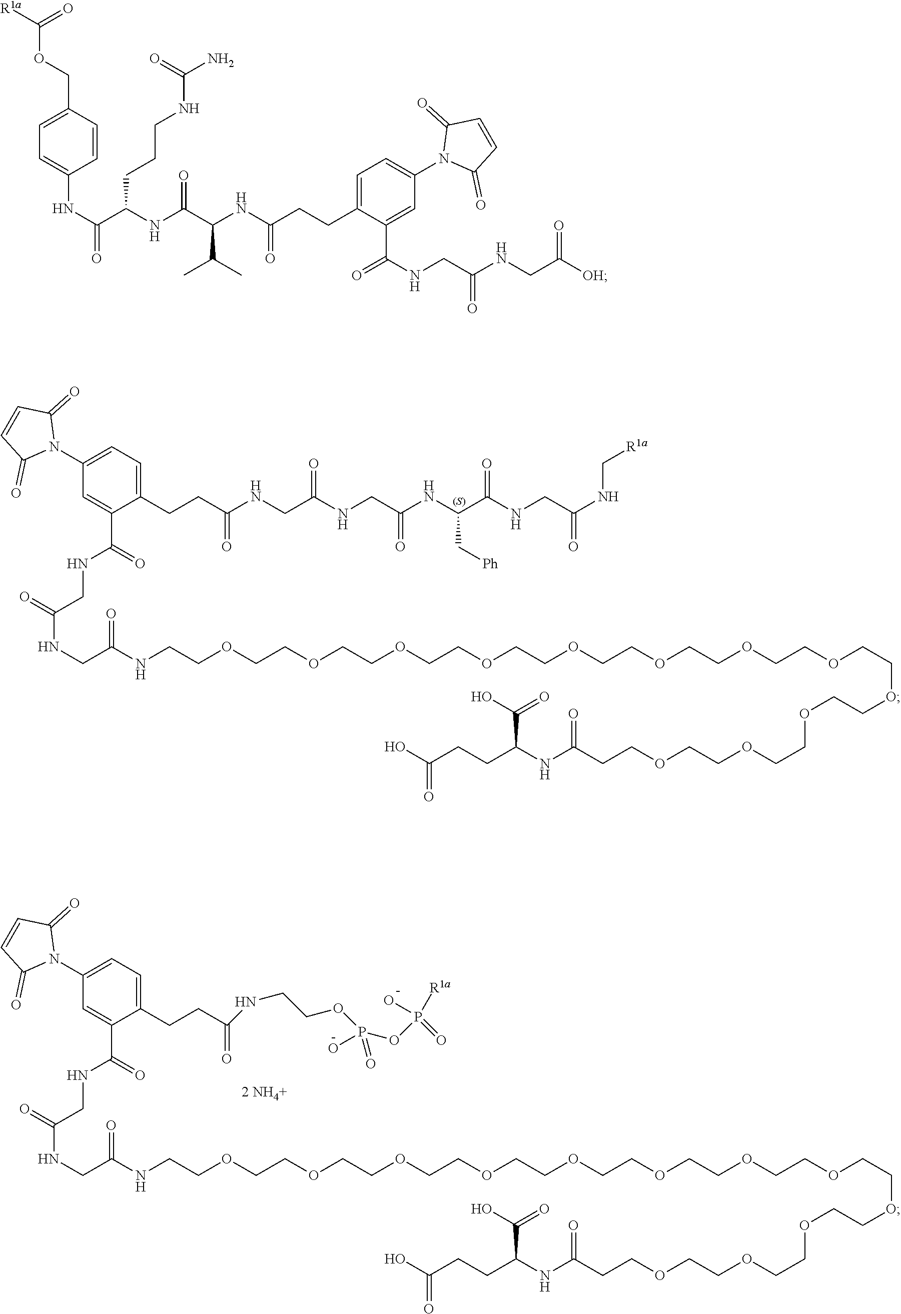

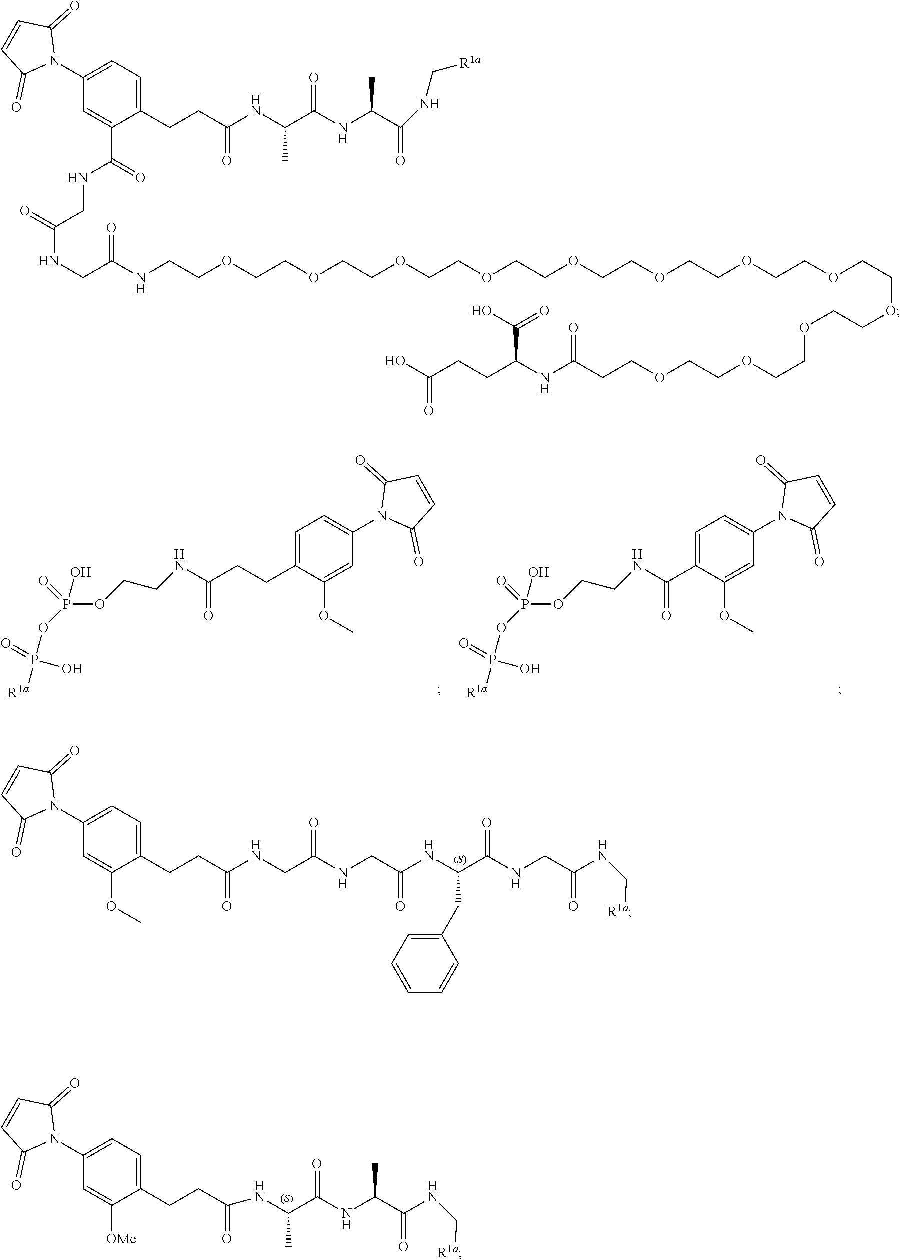

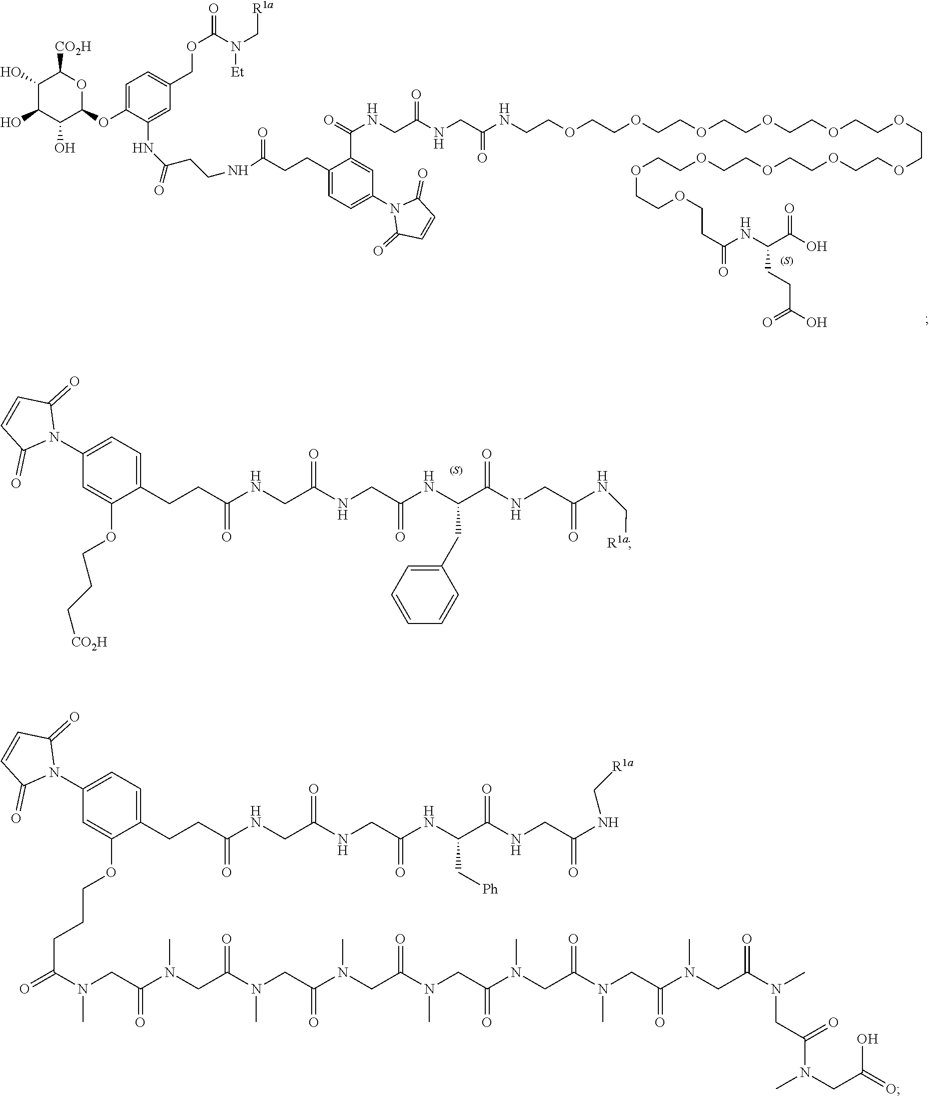

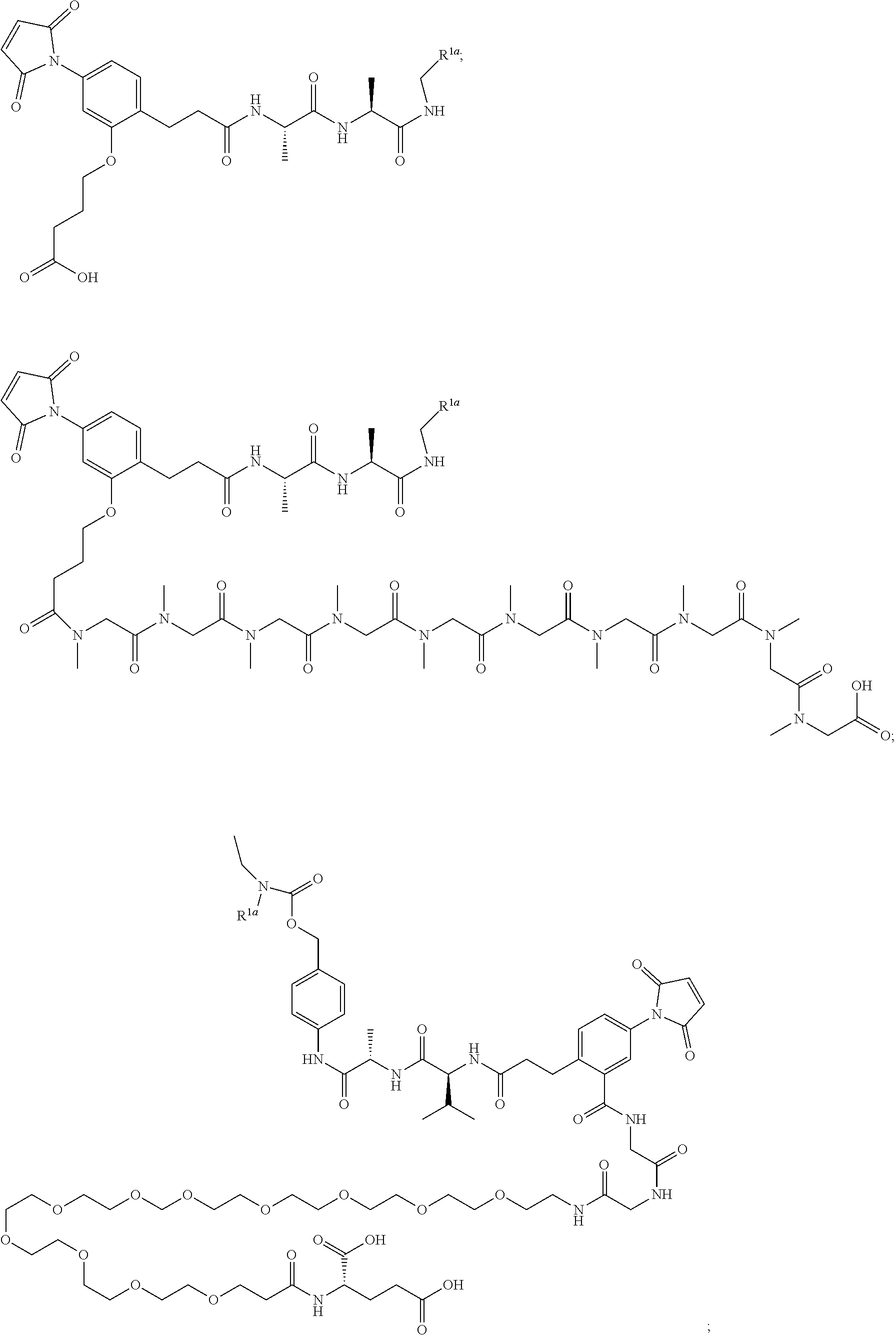

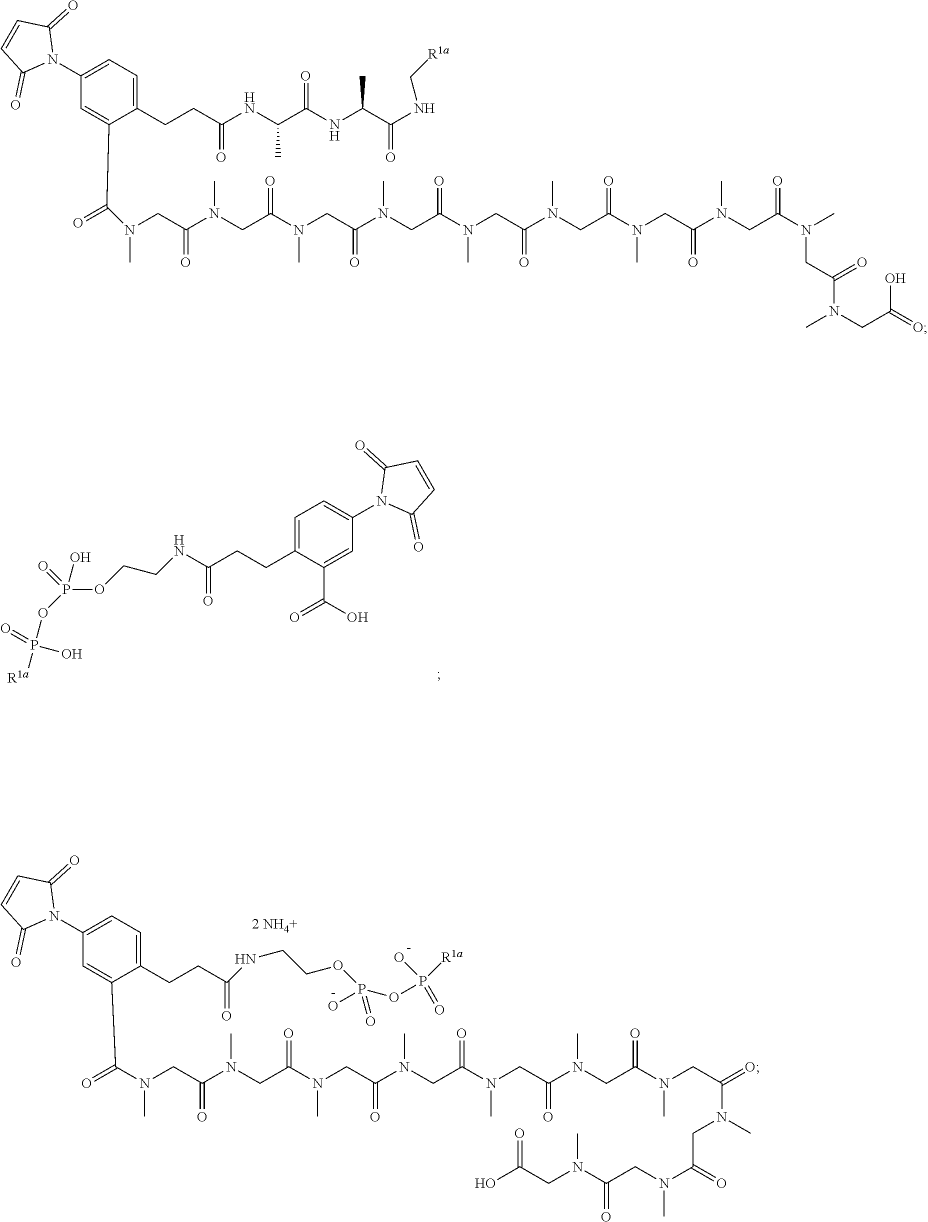

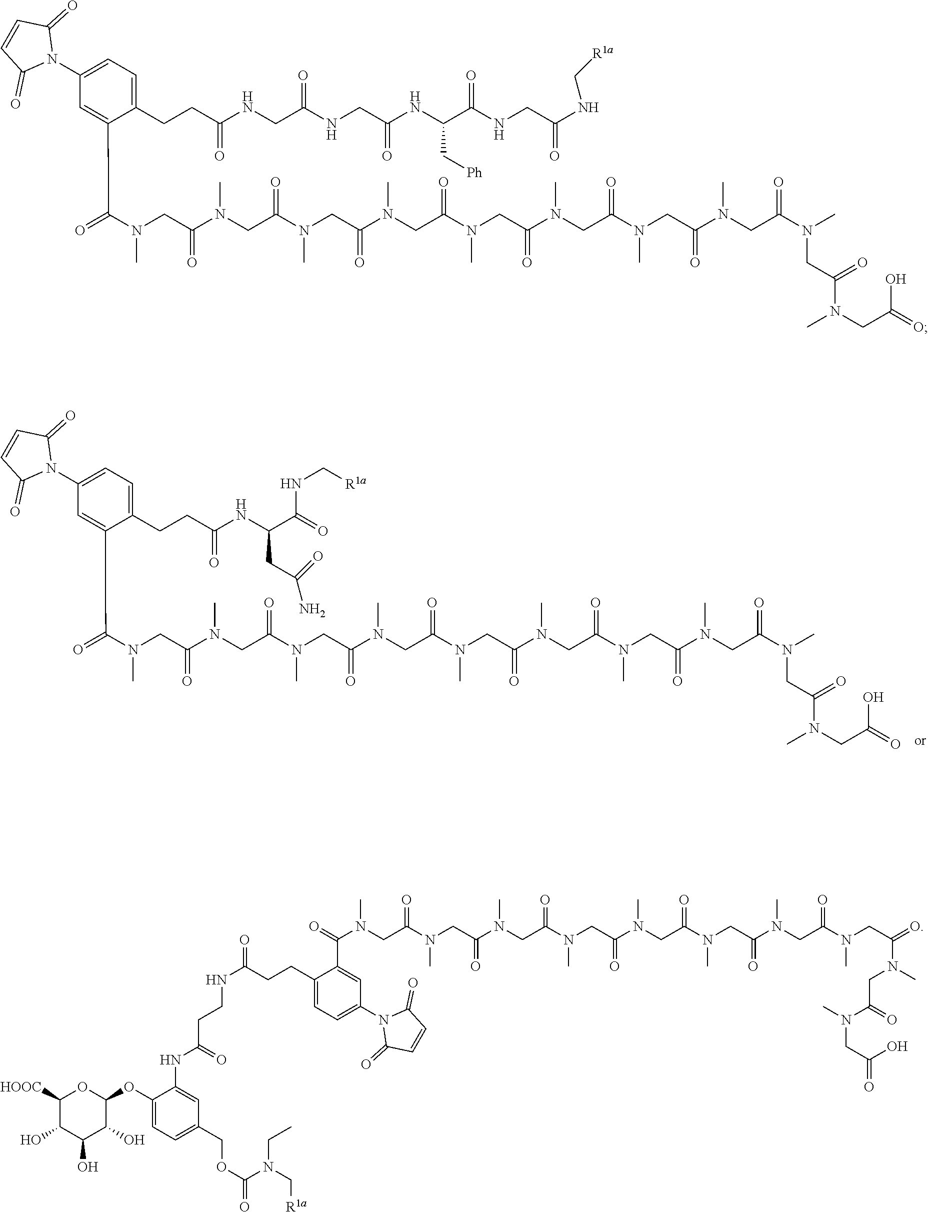

In some embodiments, the compound has one of the following structures.

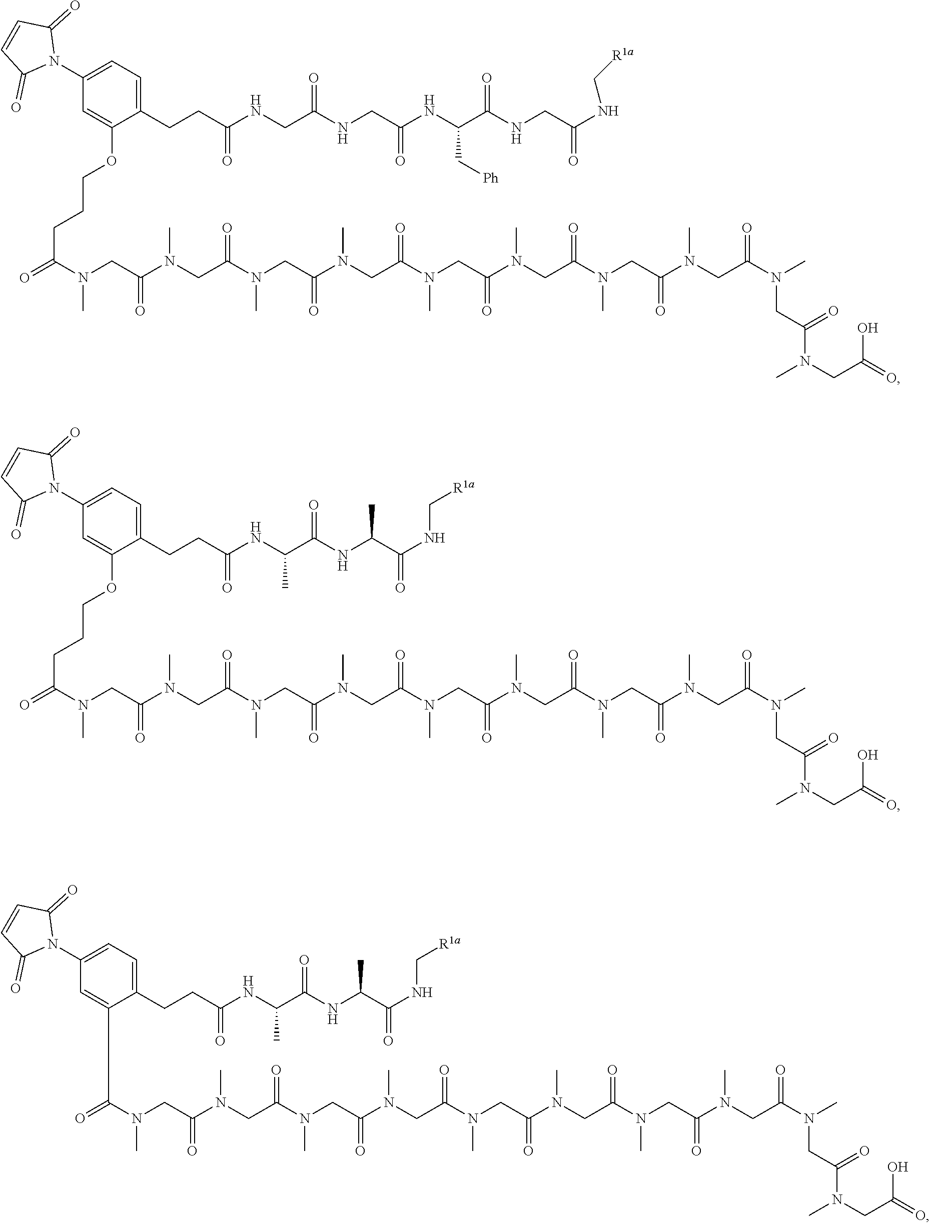

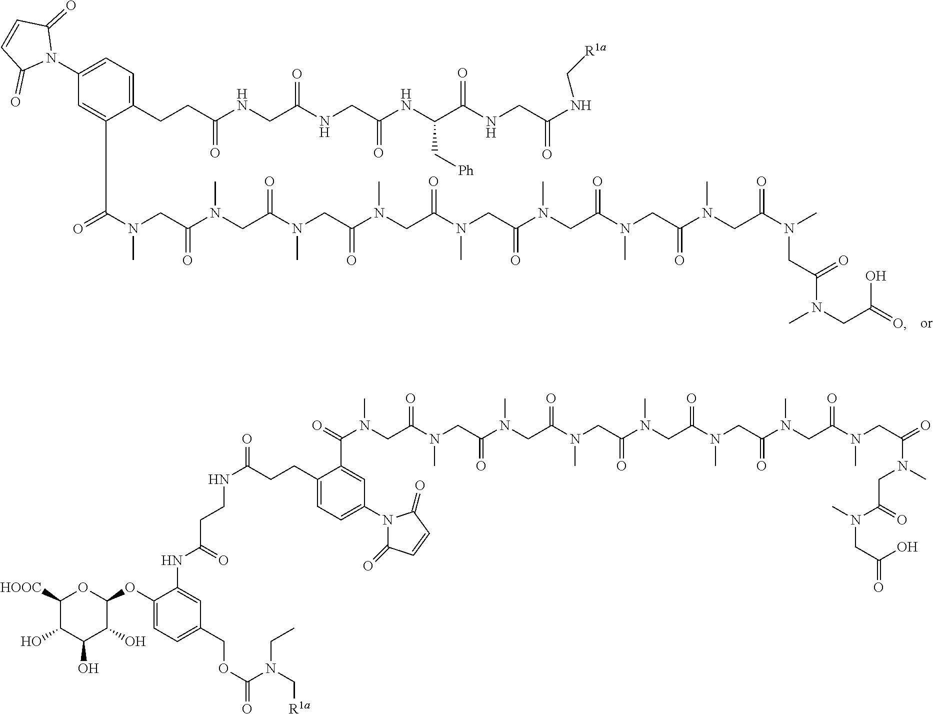

as a stereoisomer, enantiomer or tautomer thereof or a mixture thereof; or a pharmaceutically acceptable salt, solvate or prodrug thereof.

In some embodiments, the compound has one of the following structures:

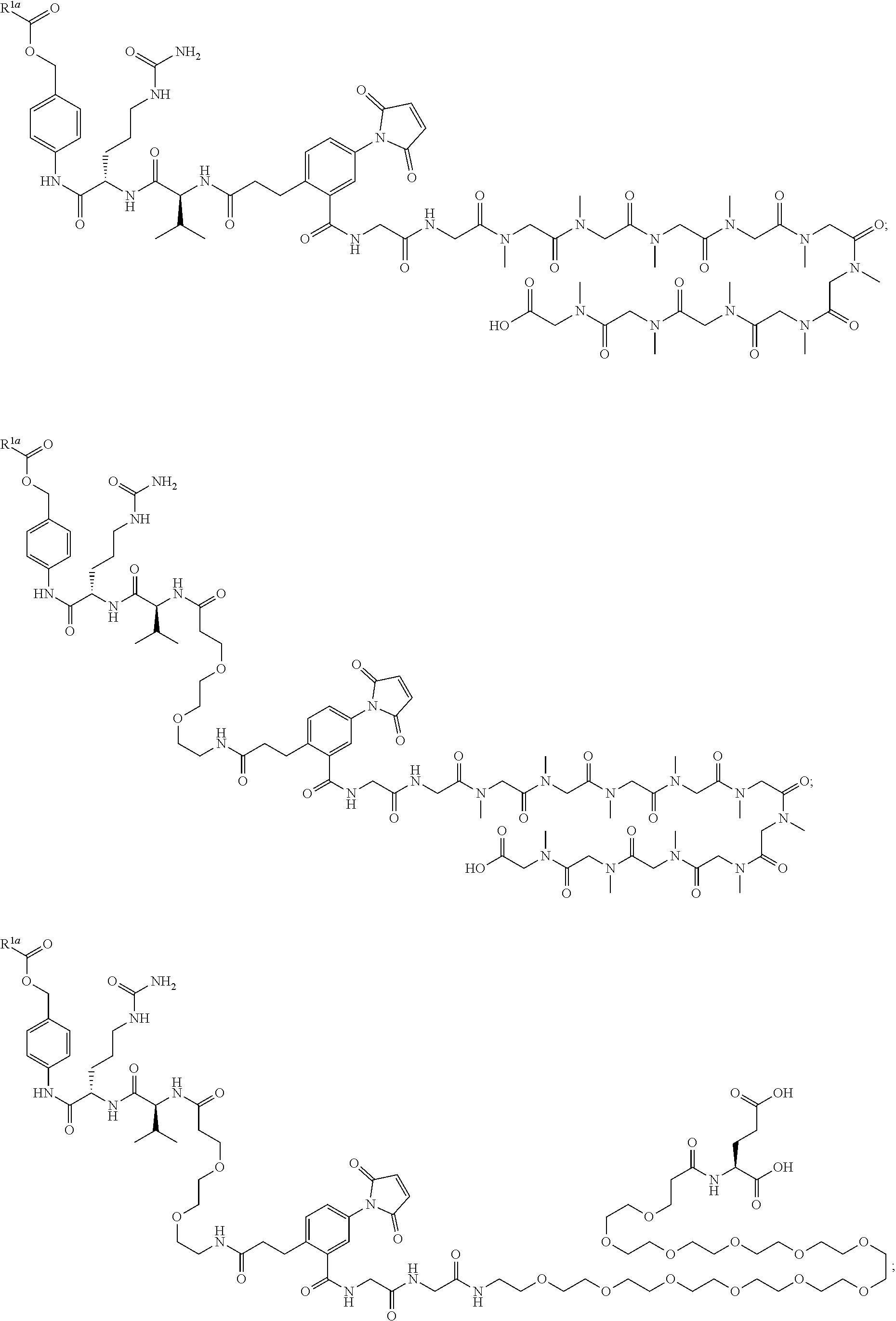

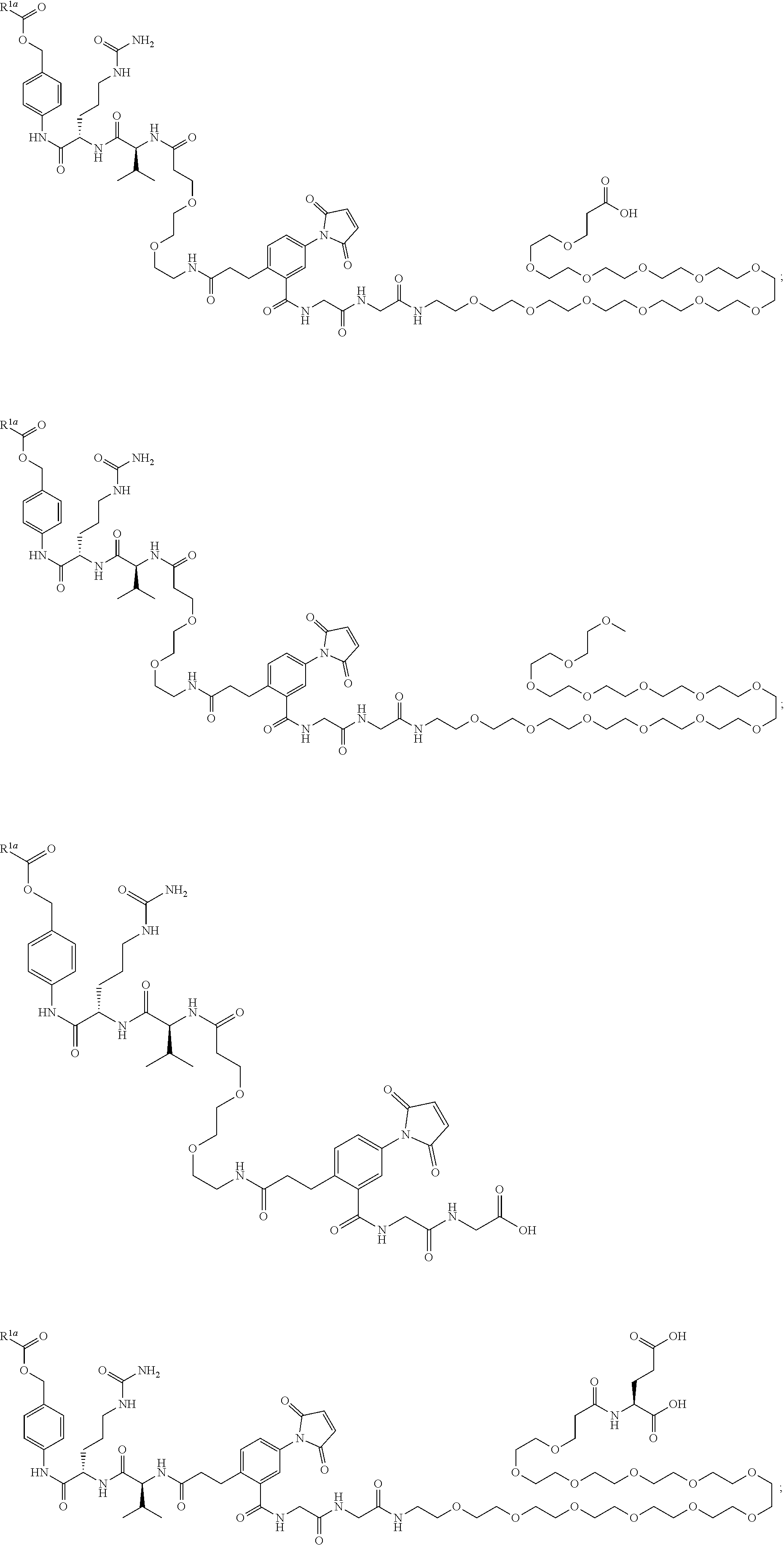

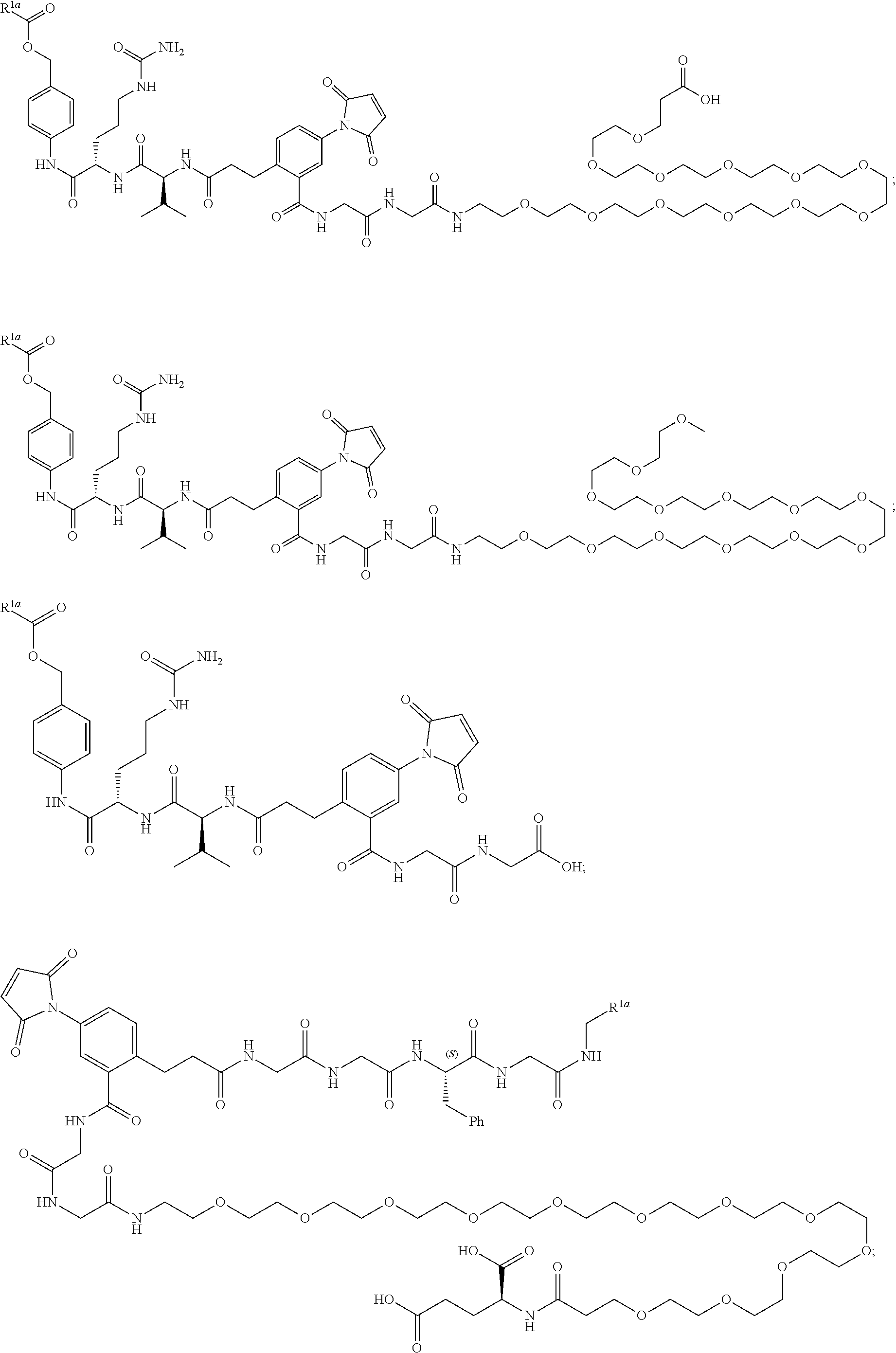

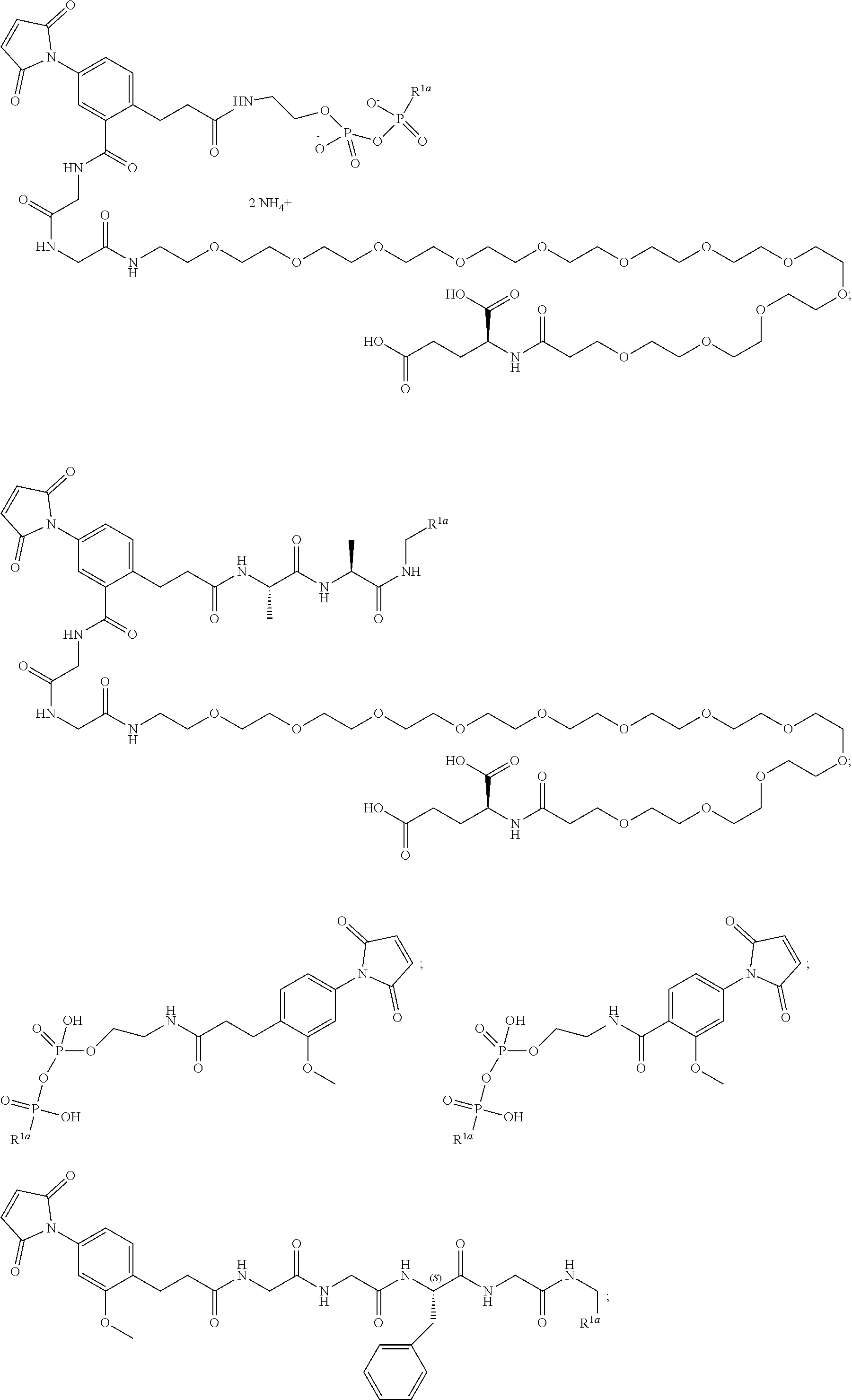

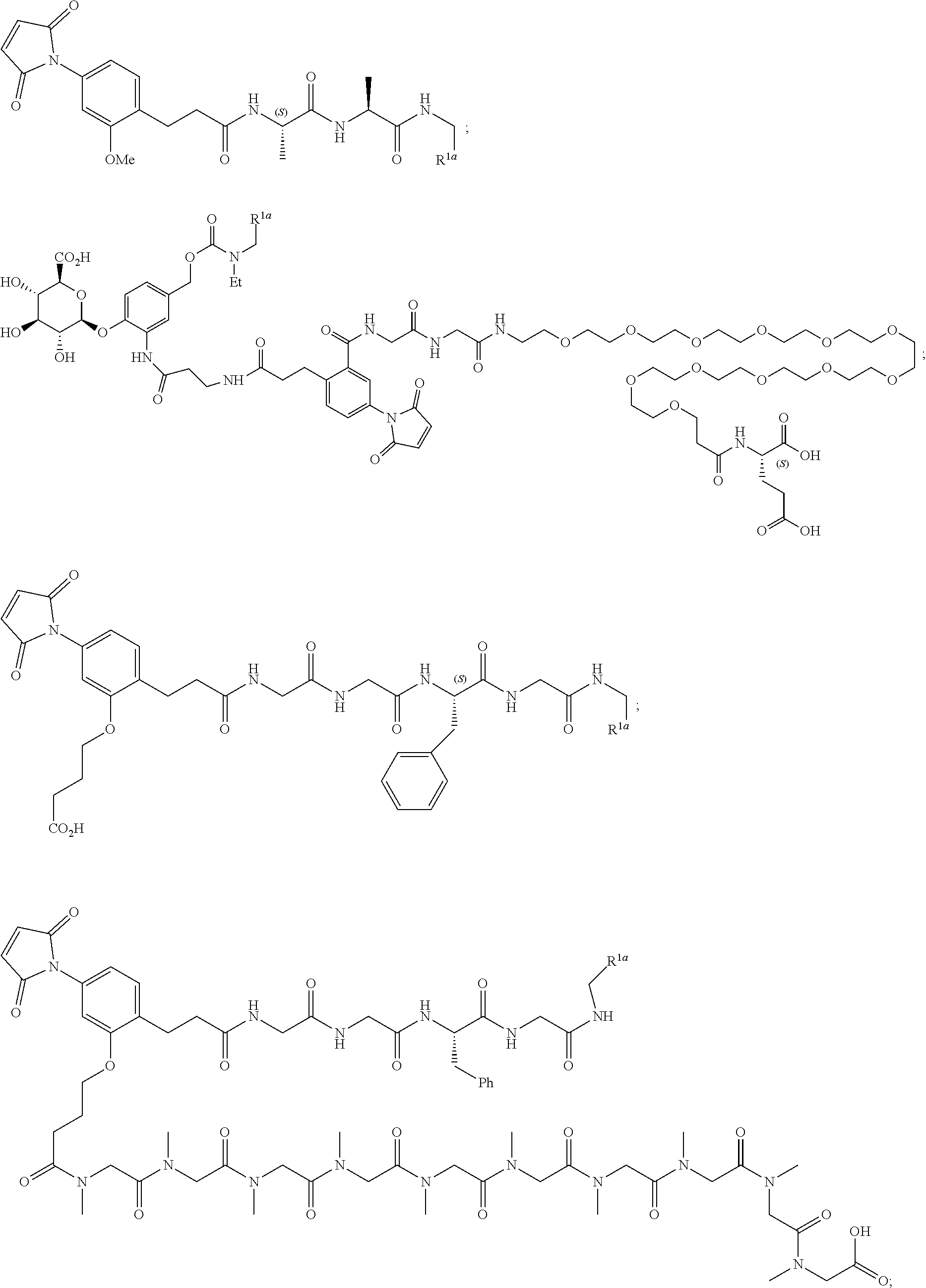

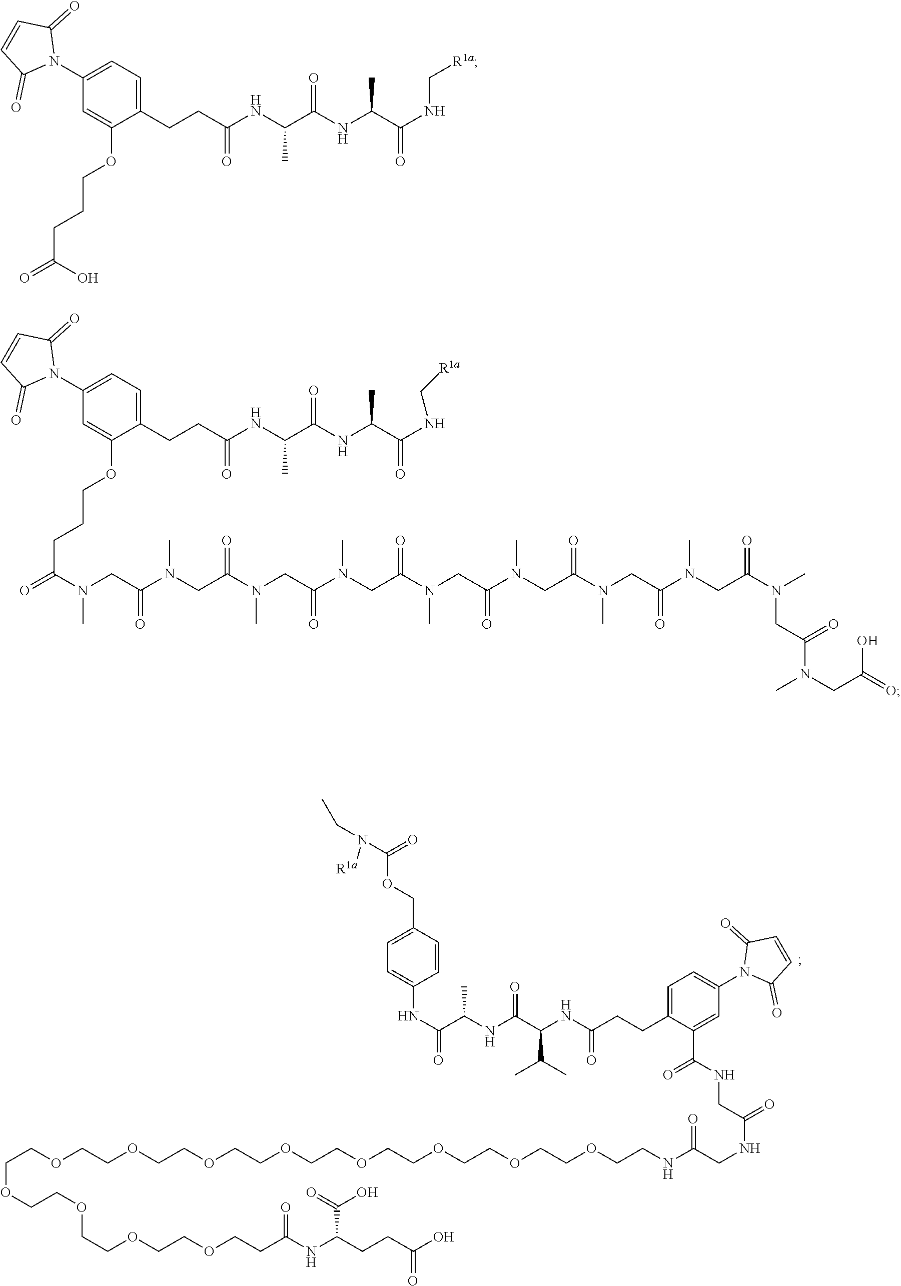

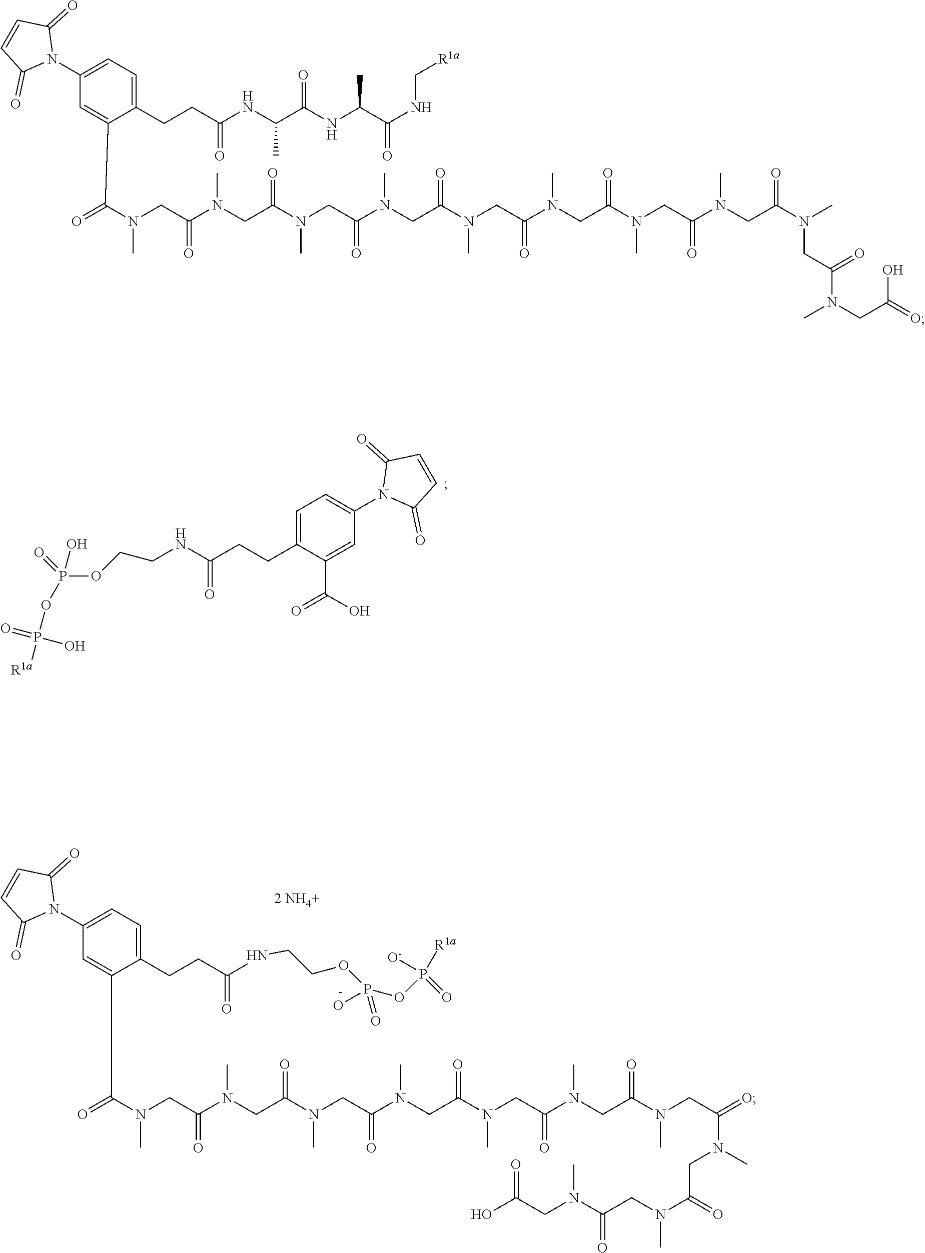

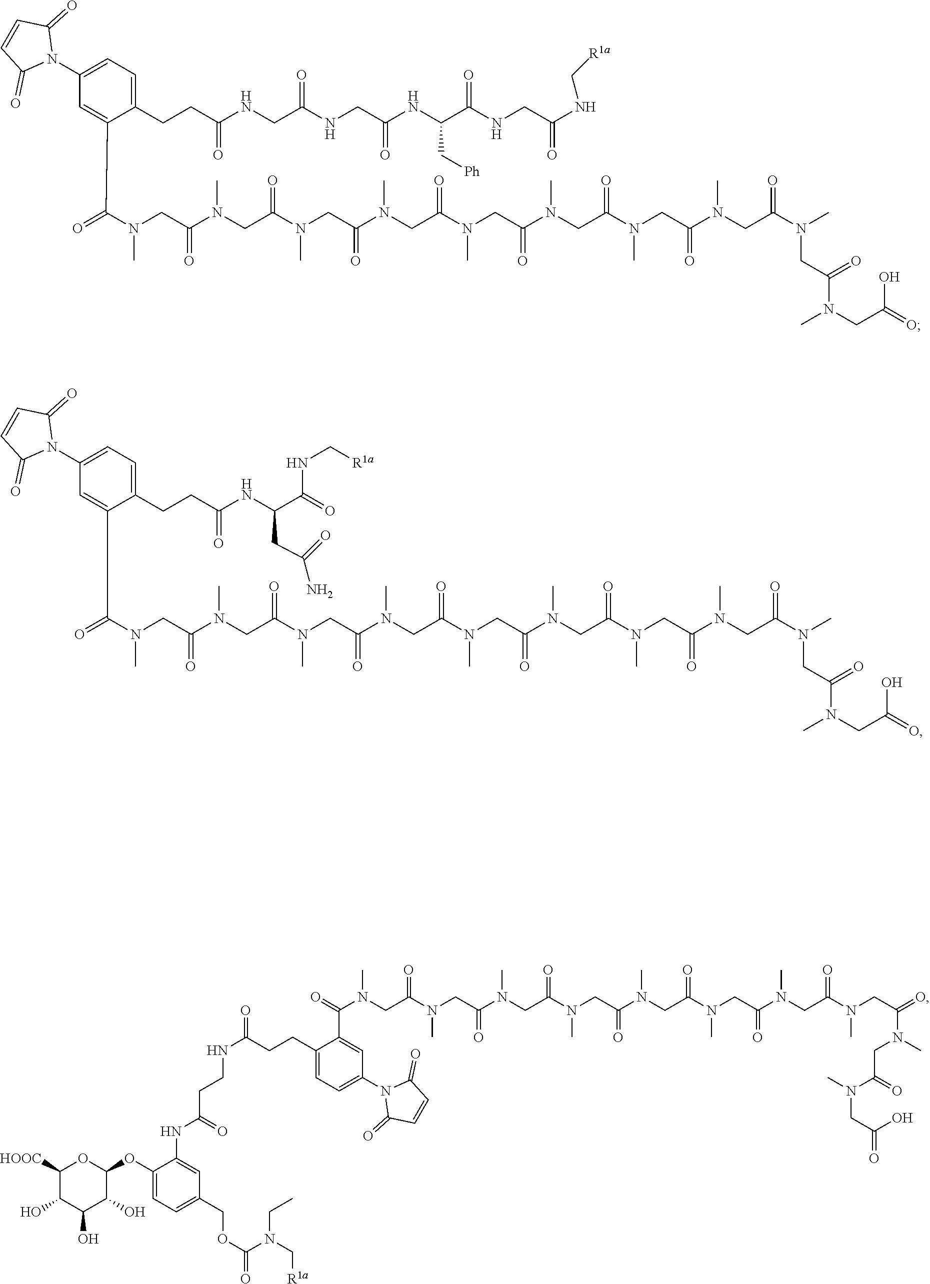

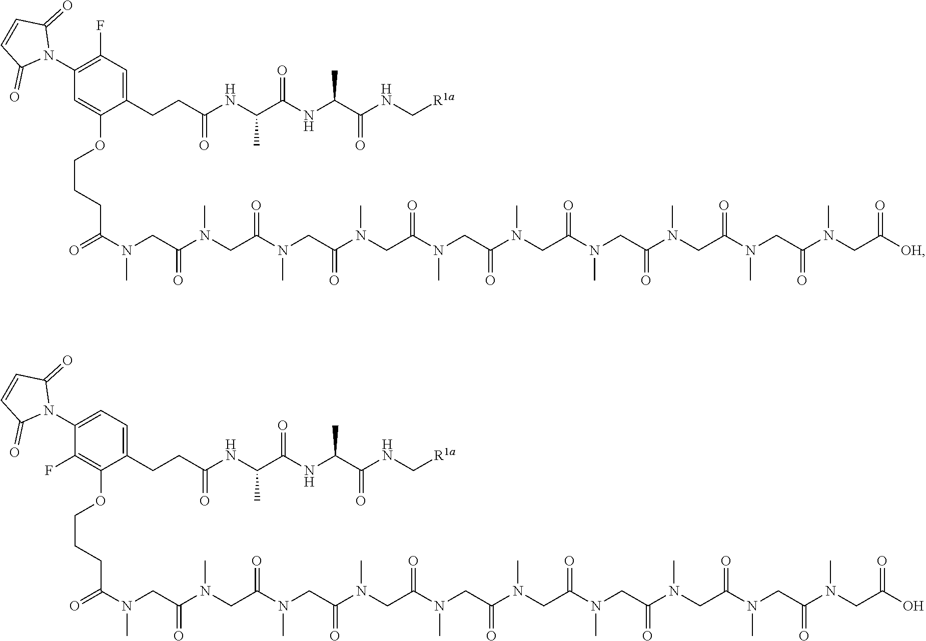

as a stereoisomer, enantiomer or tautomer thereof or a mixture thereof; or a pharmaceutically acceptable salt, solvate or prodrug thereof.

In some embodiments, a payload is a chemotherapeutic, a cytotoxic agent, or a myeloid cell agonist.

In some embodiments, a payload is a chemotherapeutic selected from the group consisting of camptothecin, paclitaxel, doxorubicin, vinblastine, dacarbazine, irinotecan, topotecan, silatecan, cositecan, Exatecan, Lurtotecan, SN-38, Dxd (i.e., CAS No. 1599440-33-1), gimatecan, Belotecan, and Rubitecan.

In certain embodiments, a payload is a cytotoxic agent selected from the group consisting of calicheamicin, anthramycin, abbeymycin, chicamycin, DC-81, mazethramycin, neothramycin A, neothramycin B, porothramycin prothracarcin, sibanomicin, sibiromycin, tomamycin, auristatin F, monomethyl auristatin F, auristatin E, monomethyl auristatin E, dolastatin, monomethyl dolastatin, mertansine, and emtansine.

In certain embodiments, a payload is a myeloid cell agonist selected from the group consisting of a STING agonist, a ligand of TLR2, a ligand of TLR3, a ligand of TLR4, a ligand of TLR5, a ligand of TLR6, a ligand of TLR7, a ligand of TLR8, a ligand of TLR9, a ligand of TLR10, a ligand of nucleotide-oligomerization domain (NOD), a ligand of an RIG-I-Like Receptors (RLR), a ligand of a C-type lectin receptor (CLR), a ligand of a Cytosolic DNA Sensor (CDS) and a ligand of an inflammasome inducer, preferably wherein the myeloid cell agonist is selected from the group consisting of selgantolimod, motolimod, resiquimod, 3M-051, 3M-052, MCT-465, IMO-4200, VTX-763, VTX-1463, RG7854, ADU-S100, MK-1454, MK-2118, BMS-986301, GSK3745417, SB-11285, and IMSA-101.

In some embodiments, a payload is an alkylating agent, an antimetabolite, a microtubule inhibitor, a topoisomerase inhibitor, a myeloid agonist, a glucocorticoid receptor agonist, or a cytotoxic antibiotic. In certain embodiments, a payload is a nitrogen mustard, a nitrosourea, a tetrazine, an aziridine, a cisplatin or cisplatin derivative, or a non-classical alkylating agent.

In certain embodiments, a payload is mechlorethamine, cyclophosphamide, melphalan, chlorambucil, ifosfamide, busulfan, N-nitroso-N-methylurea (MNU), carmustine (BCNU), lomustine (CCNU), semustine (MeCCNU), fotemustine, streptozotocin, dacarbazine, mitozolomide, temozolomide, thiotepa, mytomycin, diaziquone (AZQ), cisplatin, carboplatin, oxaliplatin, procarbazine, or hexamethylmelamine. In some embodiments, a payload is an anti-folate, a fluoropyrimidines, a deoxynucleoside analogue, or a thiopurine.

In certain embodiments, a payload is methotrexate, pemetrexed, fluorouracil, capecitabine, cytarabine, gemcitabine, decitabine, azacitidine, fludarabine, nelarabine, cladribine, clofarabine, pentostatin, thioguanine, and mercaptopurine. In some embodiments, a payload is an auristatin, a Vinca alkaloid, or a taxane. In certain embodiments, a payload is auristatin F, auristatin E, vincristine, vinblastine, vinorelbine, vindesine, vinflunine, paclitaxel, docetaxel, etoposide, or teniposide. In further embodiments, a payload is a hydrocortisone (e.g., prednisone, fluocinolone), an acetonide (e.g., budesonide, fluocinonide), a methasone-type (e.g., dexamethasone, betamethasone, fluticasone).

In some embodiments, a payload has a molecular weight greater than 150 g/mole, greater than 200 g/mole, greater than 300 g/mole, greater than 400 g/mole, greater than 500 g/mole, greater than 600 g/mole, greater than 700 g/mole, greater than 800 g/mole, greater than 900 g/mole, or greater than 1000 g/mole. In some embodiments, a payload has a molecular weight less than 150 g/mole, less than 200 g/mole, less than 300 g/mole, less than 400 g/mole, less than 500 g/mole, less than 600 g/mole, less than 700 g/mole, less than 800 g/mole, less than 900 g/mole, or less than 1000 g/mole.

Payload moieties are known to those of ordinary skill in the art. For example, payloads can be found in PCT Publication No. WO 2021/207701, WO 2019/217591, WO 2018/089373, WO 2019/136487 and U.S. Pat. No. 11,179,473, and references cited therein, the payloads from each of which are incorporated by reference herein in their entirety.

In some embodiments, the payload is a STING agonist having the following Structure (III):

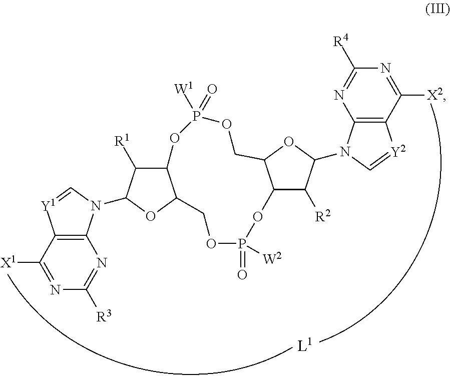

wherein:
L¹ is a bivalent linker having a first terminal end covalently bound by a single bond to X¹ and a second terminal end covalently bound by a single bond to X², wherein L¹ is C₂-C₈ alkylene, C₂-C₈ alkenylene, C₂-C₈ alkynylene, C₂-C₈ heteroalkylene, C₂-C₈ heteroalkenylene, C₂-C₈ heteroalkynylene or -(L^{1a})ₓ-L^{1b}-(L^{1c})_{y}-, each of which is optionally substituted with one or more R, wherein:
   L^{1a} and L^{1c} are each independently C₁-C₆ alkylene, C₁-C₆ alkenylene, C₂-C₆ alkynylene, C₁-C₆ heteroalkylene, C₂-C₆ heteroalkenylene, C₂-C₆ heteroalkynylene, each of which is optionally substituted with one or more R;
   L^{1b} is C₆-C₁₀ arylene, C₃-C₆ cycloalkylene, 3-6 membered heterocyclene or 5-6 membered heteroarylene, each of which is optionally substituted with one or more R; and
   x and y are each independently 0 or 1;
   R is, at each occurrence, independently R*, D, —OR^{a}, —N(R^{a})₂, —NR^{a}C(═O)R^{b}, —C(═O)N(R^{a})₂, —CO₂R^{a}, —OC(═O)R^{b}, —OC(═O)N(Rₐ)₂, —OC(═O)N(Rₐ)₂, —SR^{a}, —S(O)₂N(R^{a})₂, —S(O)₂Rₐ, C₁-C₆ alkyl, C₁-C₆ alkoxy, C₆-C₁₀ aryl, C₃-C₆ cycloalkyl, 3-6 membered heterocyclyl or 5-6 membered heteroaryl, wherein each C₁-C₆ alkyl, C₁-C₆ alkoxy, C₆-C₁₀ aryl, C₃-C₆ cycloalkyl, 3-6 membered heterocyclyl and 5-6 membered heteroaryl is independently optionally substituted with one or more substituents selected from D, —OR^{a}, —N(R^{a}), —NR^{a}C(═O)R^{b}, —C(═O)N(R^{a})₂, —CO₂R^{a}, —OC(═O)R^{b}, —OC(═O)N(Rₐ)₂, —OC(═O)N(Rₐ)₂, —SR^{a}, —S(═O)₂N(R^{a})₂ or —S(O)₂Rₐ;
X¹ and X² are each independently O or NR^{a};
   (i) W¹ and W² are independently OR^{c} or SR^{c};
      Y¹ and Y² are each N;
      R¹ is H, CN, OH, Cl, Br, N₃, C₁-C₆ alkyl or C₁-C₆ alkoxy; and
      R² is H, CN, OH, F, Cl, Br, N₃, C₁-C₆ alkyl or C₁-C₆ alkoxy,
   (ii) W¹ and W² are independently OR^{c}, SR^{c} or BHR^{c};
      at least one of Y¹ and Y² is CH, CF or CCl, and the other of Y¹ and Y² is CH, CF, CCl or N; and
      R¹ and R² are each independently H, CN, OH, F, Cl, Br, N₃, C₁-C₆ alkyl or C₁-C₆ alkoxy, or
   (iii) W¹ and W² are independently SR^{c} or BHR^{c};
      Y¹ and Y² are each N; and
      R¹ is H, CN, Cl, Br, N₃, C₁-C₆ alkyl or C₁-C₆ alkoxy; and
   R² is H, CN, OH, F, Cl, Br, N₃, C₁-C₆ alkyl or C₁-C₆ alkoxy;
   R³ and R⁴ are each independently H or —N(R^{a})₂;
   R^{a} is, at each occurrence, independently H, C₁-C₆ alkyl, C₂-C₆ alkenyl, C₁-C₆ aminylalkyl, C₂-C₆ aminylalkenyl, C₃-C₆ cycloalkyl, C₃-C₆ aminylcycloalkyl, C₃-C₆ cycloalkyl-C₁-C₆ alkyl, C₃-C₆ aminylcycloalkyl-C₁-C₆ alkyl, 3-6 membered heterocyclyl, 3-6 membered aminylheterocyclyl, 3-6 membered heterocyclyl-C₁-C₆ alkyl or 3-6 membered aminylheterocyclyl-C₁-C₆ alkyl, wherein each substitutable R^{a} is optionally substituted with R*;
   R^{b} is, at each occurrence, independently, C₁-C₆ alkyl, C₂-C₆ alkenyl, C₁-C₆ aminylalkyl, C₂-C₆ aminylalkenyl, C₃-C₆ cycloalkyl, C₃-C₆ aminylcycloalkyl, C₃-C₆ cycloalkyl-C₁-C₆ alkyl, C₃-C₆ aminylcycloalkyl-C₁-C₆ alkyl, 3-6 membered heterocyclyl, 3-6 membered aminylheterocyclyl, 3-6 membered heterocyclyl-C₁-C₆ alkyl or 3-6 membered aminylheterocyclyl-C₁-C₆ alkyl, wherein each R^{b} is optionally substituted with R*;
   R^{c} is, at each occurrence, independently H, C₁-C₆ alkyl, —CH₂OC(═O)R^{d} or —R^{e}R^{f}, wherein each substitutable R^{c} is optionally substituted with R*;
   R^{d} is, at each occurrence, independently C₁-C₆ alkyl, wherein each R^{d} is optionally substituted with R*;
   R^{e} is, at each occurrence, independently absent, C₁-C₆ alkylene or C₁-C₆ heteroalkylene, wherein each alkylene and heteroalkyl is optionally substituted with one or more R^{g};
   R^{f} is, at each occurrence, independently H, C₁-C₂₀ alkyl C₁-C₂₀ heteroalkyl —OC(O)OC₁-C₂₀ alkyl, cycloalkyl, heterocyclyl, aryl, or heteroaryl, wherein each alkyl, heteroalkyl, cycloalkyl, heterocyclyl, aryl, and heteroaryl is optionally substituted with one or more R^{h};
   R^{g} is, at each occurrence, independently R*, halo, —CN, C₁-C₂₀ alkyl, —ORⁱ, oxo, cycloalkyl, heterocyclyl, aryl, or heteroaryl, wherein each alkyl, cycloalkyl, heterocyclyl, aryl, and heteroaryl is optionally substituted with one or more R^{j};
   R^{h} is, at each occurrence, independently R*, C₁-C₂₀ alkyl, C₁-C₂₀ heteroalkyl, —C(═O)C₁-C₂₀ alkyl, —OC(═O)C₁-C₂₀ alkyl, —C(═O)OC₁-C₂₀ alkyl, —OC(═O)OC₁-C₂₀ alkyl, —C(═O)N(R^{k})C₁-C₂₀ alkyl, —N(R^{k})C(═O)C₁-C₂₀ alkyl, —OC(═O)N(R^{k})C₁-C₂₀ alkyl, —C(═O)C₁-C₂₀ heteroalkyl, —OC(═O)C₁-C₂₀ heteroalkyl, —C(═O)OC₁-C₂₀ heteroalkyl, —OC(═O)OC₁-C₂₀ heteroalkyl, —C(═O)N(R^{k})C₁-C₂₀ heteroalkyl, —N(R^{k})C(═O)C₁-C₂₀ heteroalkyl, —OC(═O)N(R)C₁-C₂₀ heteroalkyl, —Oaryl, —Oheteroaryl, —C(═O)aryl, —C(═O)heteroaryl, —O(═O)aryl, —C(═O)Oaryl, —OC(═O)heteroaryl, —C(═O)Oheteroaryl, —C(═O)N(R^{k})aryl, —C(═O)N(R^{k})heteroaryl, —N(R^{k})C(═O)aryl, —N(R^{k})C(═O)heteroaryl, —OC(═O)N(R^{k})aryl, —OC(═O)N(R^{k})heteroaryl, —N(R^{k})C(═O)Oaryl, —N(R^{k})C(═O)Oheteroaryl, —S(═O)₂N(R^{k})aryl, —S(═O)₂N(R^{k})heteroaryl or —N(R^{k})-*peptide-L², wherein * is the C-terminal of the peptide, and L² is absent, —C₁-C₁₂ alkyl, —C₁-C₁₂ heteroalkyl, —C(═O)C₁-C₁₂ alkyl or —C(═O)C₁-C₁₂ heteroalkyl, and wherein each alkyl, heteroalkyl, aryl, and heteroaryl is optionally substituted by one or more R^{j};
   Rⁱ is, at each occurrence, independently H, C₁-C₂₀ alkyl, C₁-C₂₀ heteroalkyl cycloalkyl, heterocyclyl, aryl, or heteroaryl, wherein each alkyl, cycloalkyl, heterocyclyl, aryl, and heteroaryl is optionally substituted with one or more R^{j};
   R^{j} is, at each occurrence, independently R*, C₁-C₂₀ alkyl, —OC₁-C₂₀ alkyl, C₁-C₂₀ heteroalkyl, halo, —CN, —OH, oxo, aryl, heteroaryl, —Oaryl or —Oheteroaryl;
   R^{k} is, at each occurrence, independently H or C₁-C₂₀ alkyl; and
R* is is a bivalent linker having a first terminal end covalently bound by a single bond to the remainder of Structure (I) or Structure (II) and a second terminal end covalently bound by a single bond to at least one of L¹, X¹, X², R³, R⁴, W¹ or W² is substituted with R*.

In other embodiments, the STING agonist has the following Structure (IIIA):

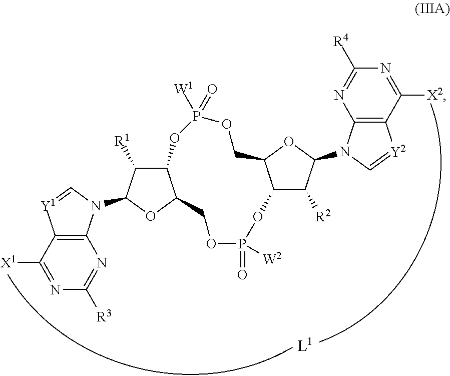

In still different embodiments, the STING agonist is a compound having the following Structure (IIIA′):

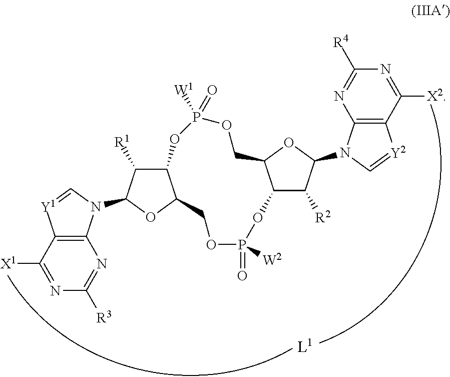

In other embodiments, the disclosure provides a compound having activity as a STING agonist and having the following Structure (IV):

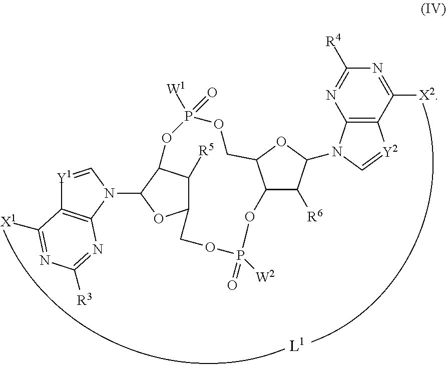

or a pharmaceutically acceptable salt, tautomer, prodrug or stereoisomer thereof, wherein:
L¹ is a bivalent linker having a first terminal end covalently bound by a single bond to X¹ and a second terminal end covalently bound by a single bond to X², wherein L¹ is C₂-C₈ alkylene, C₂-C₈ alkenylene, C₂-C₈ alkynylene, C₂-C₈ heteroalkylene, C₂-C₈ heteroalkenylene, C₂-C₈ heteroalkynylene or -(L^{1a})ₓ-L^{1b}-(L^{1c})_{y}-, each of which is optionally substituted with one or more R, wherein:
   L^{1a} and L^{1c} are each independently C₁-C₆ alkylene, C₁-C₆ alkenylene, C₂-C₆ alkynylene, C₁-C₆ heteroalkylene, C₂-C₆ heteroalkenylene, C₂-C₆ heteroalkynylene, each of which is optionally substituted with one or more R;
   L^{1b} is C₆-C₁₀ arylene, C₃-C₆ cycloalkylene, 3-6 membered heterocyclene or 5-6 membered heteroarylene, each of which is optionally substituted with one or more R; and
   x and y are each independently 0 or 1;
R is, at each occurrence, independently R*, D, —OR^{a}, —N(R^{a})₂, —NR^{a}C(═O)R^{b}, —C(═O)N(R^{a})₂, —CO₂R^{a}, —OC(═O)R^{b}, —OC(═O)N(Rₐ)₂, —OC(═O)N(Rₐ)₂, —SR^{a}, —S(O)₂N(R^{a})₂, —S(O)₂Rₐ, C₁-C₆ alkyl, C₁-C₆ alkoxy, C₆-C₁₀ aryl, C₃-C₆ cycloalkyl, 3-6 membered heterocyclyl or 5-6 membered heteroaryl, wherein each C₁-C₆ alkyl, C₁-C₆ alkoxy, C₆-C₁₀ aryl, C₃-C₆ cycloalkyl, 3-6 membered heterocyclyl and 5-6 membered heteroaryl is independently optionally substituted with one or more substituents selected from D, —OR^{a}, —N(R^{a})₂, —NR^{a}C(═O)R^{b}, —C(═O)N(R^{a})₂, —CO₂R^{a}, —OC(═O)R^{b}, —OC(═O)N(Rₐ)₂, —OC(═O)N(Rₐ)₂, —SR^{a}, —S(═O)₂N(R^{a})₂ or —S(O)₂Rₐ;
W¹ and W² are each independently OR^{c}, SR^{c} or BHR^{c};
X¹ and X² are each independently O or NR^{a};
   (i) Y¹ and Y² are each N;
      R⁵ is H, CN, OH, F, Cl, Br, N₃, C₁-C₆ alkyl or C₁-C₆ alkoxy; and
      R⁶ is H, CN, F, Cl, Br, N₃, C₁-C₆ alkyl or C₁-C₆ alkoxy, or,
   (ii) at least one of Y¹ and Y² is CH, CF or CCl, and the other of Y¹ and Y² is CH, CF, CCl or N; and
      R⁵ and R⁶ are each independently H, CN, OH, F, Cl, Br, N₃, C₁-C₆ alkyl or C₁-C₆ alkoxy,
R³ and R⁴ are each independently H or —N(R^{a})₂;
R^{a} is, at each occurrence, independently H, C₁-C₆ alkyl, C₂-C₆ alkenyl, C₁-C₆ aminylalkyl, C₂-C₆ aminylalkenyl, C₃-C₆ cycloalkyl, C₃-C₆ aminylcycloalkyl, C₃-C₆ cycloalkyl-C₁-C₆ alkyl, C₃-C₆ aminylcycloalkyl-C₁-C₆ alkyl, 3-6 membered heterocyclyl, 3-6 membered aminylheterocyclyl, 3-6 membered heterocyclyl-C₁-C₆ alkyl or 3-6 membered aminylheterocyclyl-C₁-C₆ alkyl, wherein each substitutable R^{a} is optionally substituted with R*;
R^{b} is, at each occurrence, independently, C₁-C₆ alkyl, C₂-C₆ alkenyl, C₁-C₆ aminylalkyl, C₂-C₆ aminylalkenyl, C₃-C₆ cycloalkyl, C₃-C₆ aminylcycloalkyl, C₃-C₆ cycloalkyl-C₁-C₆ alkyl, C₃-C₆ aminylcycloalkyl-C₁-C₆ alkyl, 3-6 membered heterocyclyl, 3-6 membered aminylheterocyclyl, 3-6 membered heterocyclyl-C₁-C₆ alkyl or 3-6 membered aminylheterocyclyl-C₁-C₆ alkyl, wherein each R^{b} is optionally substituted with R*;
R^{c} is, at each occurrence, independently H, C₁-C₆ alkyl, —CH₂OC(═O)R^{d} or —R^{e}R^{f}, wherein each substitutable R^{c} is optionally substituted with R*;
R^{d} is, at each occurrence, independently C₁-C₆ alkyl, wherein each R^{d} is optionally substituted with R*;
R^{e} is, at each occurrence, independently absent, C₁-C₆ alkylene or C₁-C₆ heteroalkylene, wherein each alkylene and heteroalkyl is optionally substituted with one or more R^{g};
R^{f} is, at each occurrence, independently H, C₁-C₂₀ alkyl C₁-C₂₀ heteroalkyl —OC(O)OC₁-C₂₀ alkyl, cycloalkyl, heterocyclyl, aryl, or heteroaryl, wherein each alkyl, heteroalkyl, cycloalkyl, heterocyclyl, aryl, and heteroaryl is optionally substituted with one or more R^{h};
R^{g} is, at each occurrence, independently R*, halo, —CN, C₁-C₂₀ alkyl, —ORⁱ, oxo, cycloalkyl, heterocyclyl, aryl, or heteroaryl, wherein each alkyl, cycloalkyl, heterocyclyl, aryl, and heteroaryl is optionally substituted with one or more R^{j};
R^{h} is, at each occurrence, independently R*, C₁-C₂₀ alkyl, C₁-C₂₀ heteroalkyl, —C(═O)C₁-C₂₀ alkyl, —OC(═O)C₁-C₂₀ alkyl, —C(═O)OC₁-C₂₀ alkyl, —OC(═O)OC₁-C₂₀ alkyl, —C(═O)N(R^{k})C₁-C₂₀ alkyl, —N(R^{k})C(═O)C₁-C₂₀ alkyl, —OC(═O)N(R^{k})C₁-C₂₀ alkyl, —C(═O)C₁-C₂₀ heteroalkyl, —OC(═O)C₁-C₂₀ heteroalkyl, —C(═O)OC₁-C₂₀ heteroalkyl, —OC(═O)OC₁-C₂₀ heteroalkyl, —C(═O)N(R^{k})C₁-C₂₀ heteroalkyl, —N(R^{k})C(═O)C₁-C₂₀ heteroalkyl, —OC(═O)N(R^{k})C₁-C₂₀ heteroalkyl, —Oaryl, —Oheteroaryl, —C(═O)aryl, —C(═O)heteroaryl, —OC(═O)aryl, —C(═O)Oary, —OC(═O)heteroaryl, —C(═O)Oheteroaryl, —C(═O)N(R^{k})aryl, —C(═O)N(R^{k})heteroaryl, —N(R^{k})C(═O)aryl, —N(R^{k})C(═O)heteroaryl, —OC(═O)N(R^{k})aryl, —OC(═O)N(R^{k})heteroaryl, —N(R^{k})C(═O)Oaryl, —N(R^{k})C(═O)Oheteroaryl, —S(═O)₂N(R^{k})aryl, —S(═O)₂N(R^{k})heteroaryl or —N(R^{k})-*peptide-L², wherein * is the C-terminal of the peptide, and L² is absent, —C₁-C₁₂ alkyl, —C₁-C₁₂ heteroalkyl, —C(═O)C₁-C₁₂ alkyl or —C(═O)C₁-C₁₂ heteroalkyl, and wherein each alkyl, heteroalkyl, aryl, and heteroaryl is optionally substituted by one or more R^{j};
Rⁱ is, at each occurrence, independently H, C₁-C₂₀ alkyl, C₁-C₂₀ heteroalkyl cycloalkyl, heterocyclyl, aryl, or heteroaryl, wherein each alkyl, cycloalkyl, heterocyclyl, aryl, and heteroaryl is optionally substituted with one or more R^{j};
R^{j} is, at each occurrence, independently R*, C₁-C₂₀ alkyl, —OC₁-C₂₀ alkyl, C₁-C₂₀ heteroalkyl, halo, —CN, —OH, oxo, aryl, heteroaryl, —Oaryl or —Oheteroaryl;
R^{k} is, at each occurrence, independently H or C₁-C₂₀ alkyl; and
R* is is a bivalent linker having a first terminal end covalently bound by a single bond to the remainder of Structure (I) or Structure (II) and a second terminal end covalently bound by a single bond to at least one of L¹, X¹, X², R³, R⁴, W¹ or W² is substituted with R*.

In other embodiments, a STING agonist of the disclosure has the following Structure (IVA):

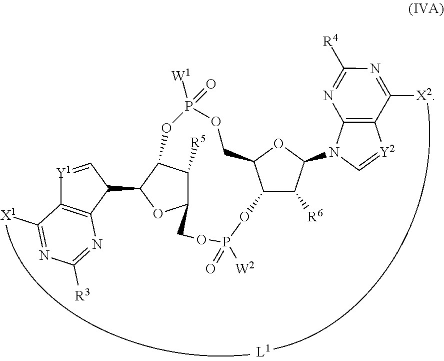

In other embodiments, the STING agonist has the following Structure (IVA′):

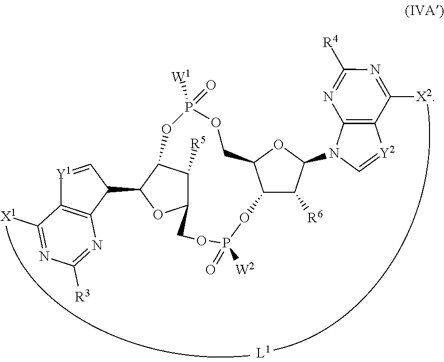

In some embodiments, R^{1a} has one of the following structures:

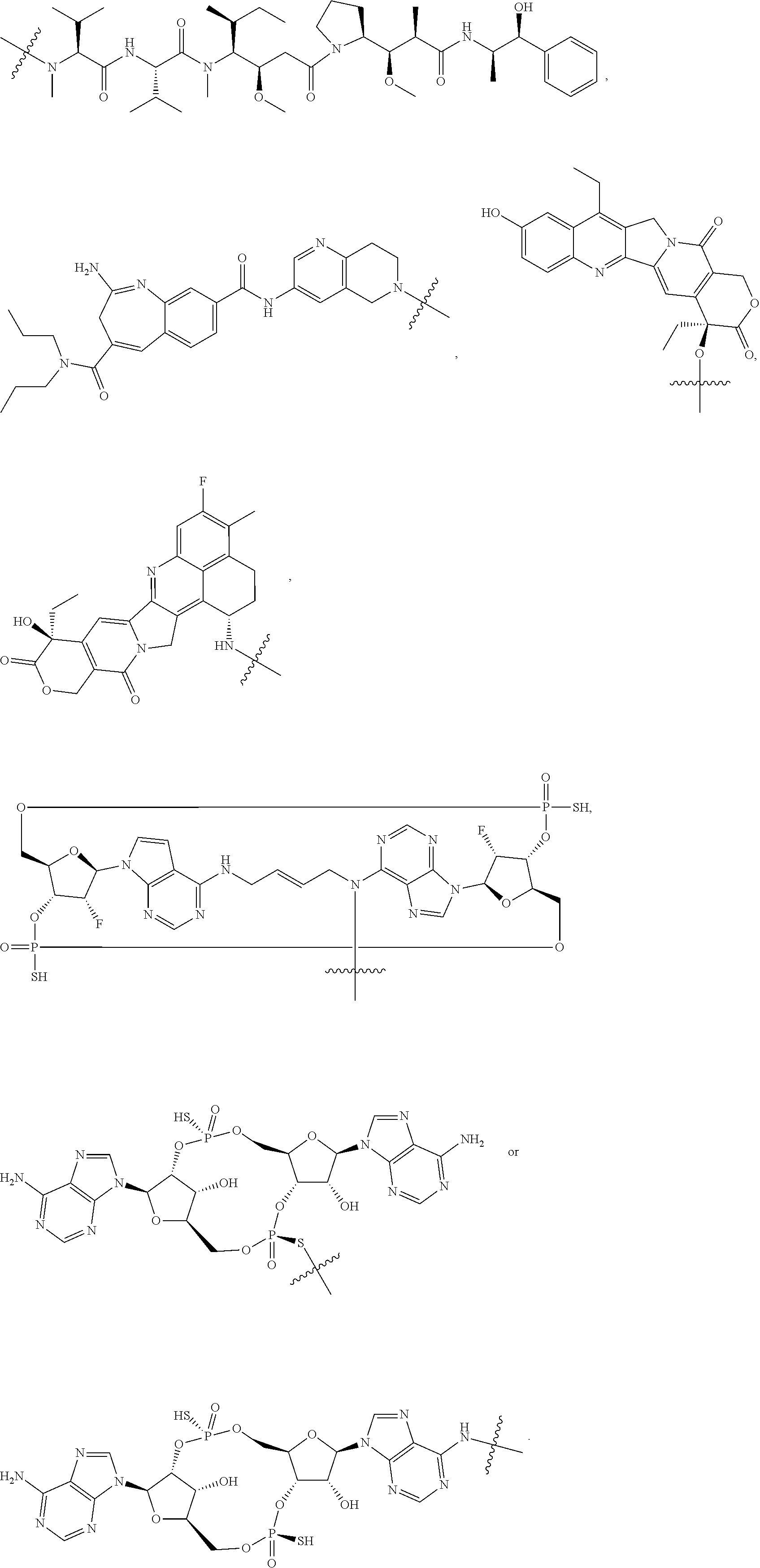

In some embodiments, R^{1a} has one of the following structures:

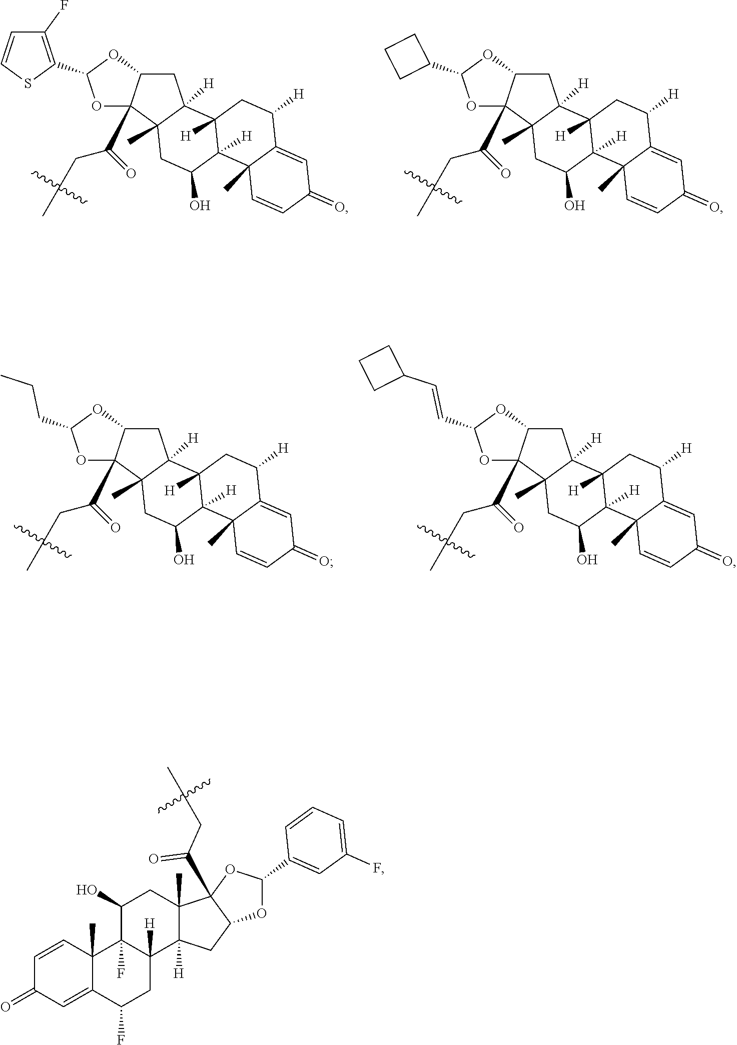

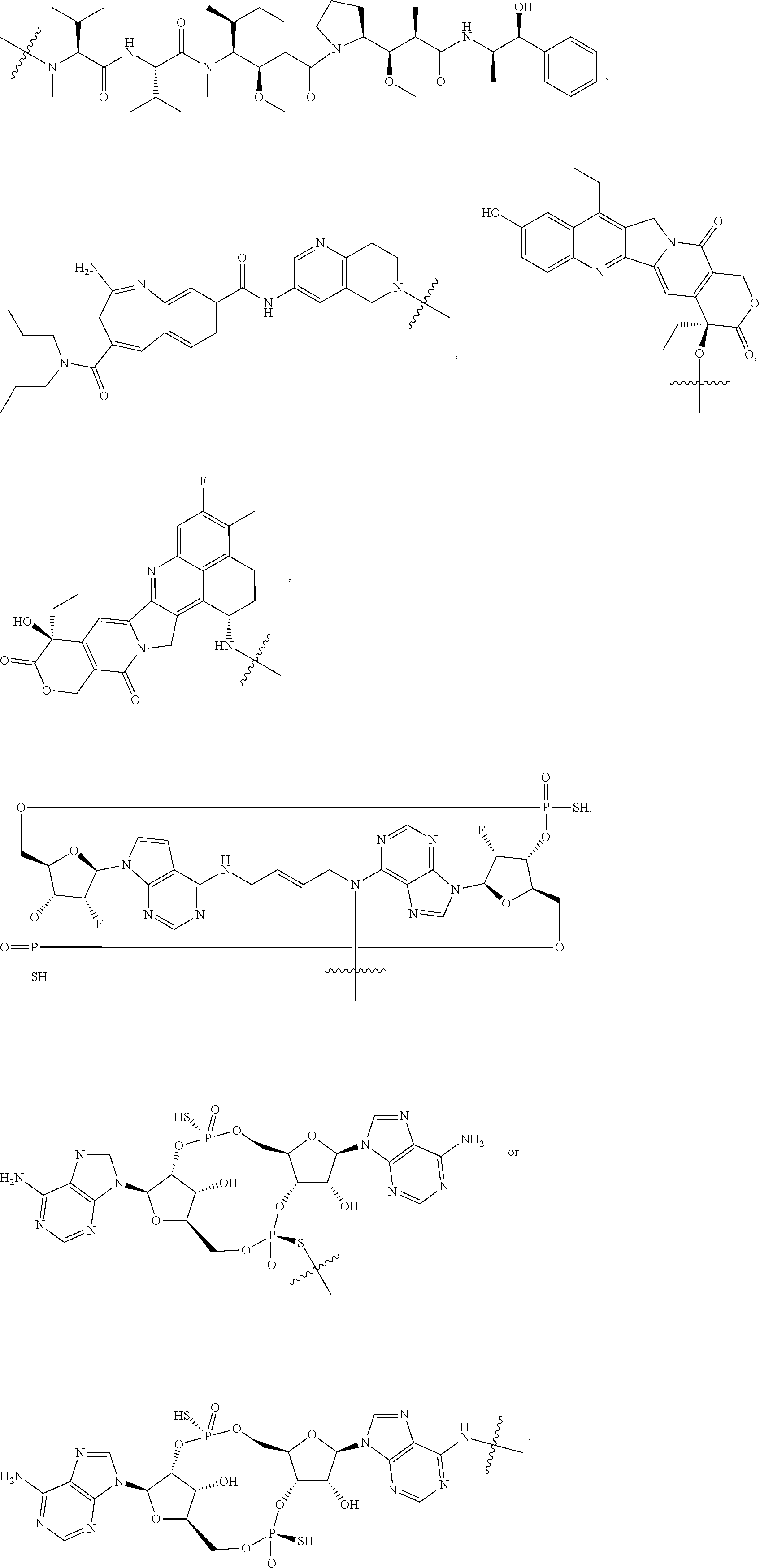

In some embodiments, R^{1a} has one of the following structures.

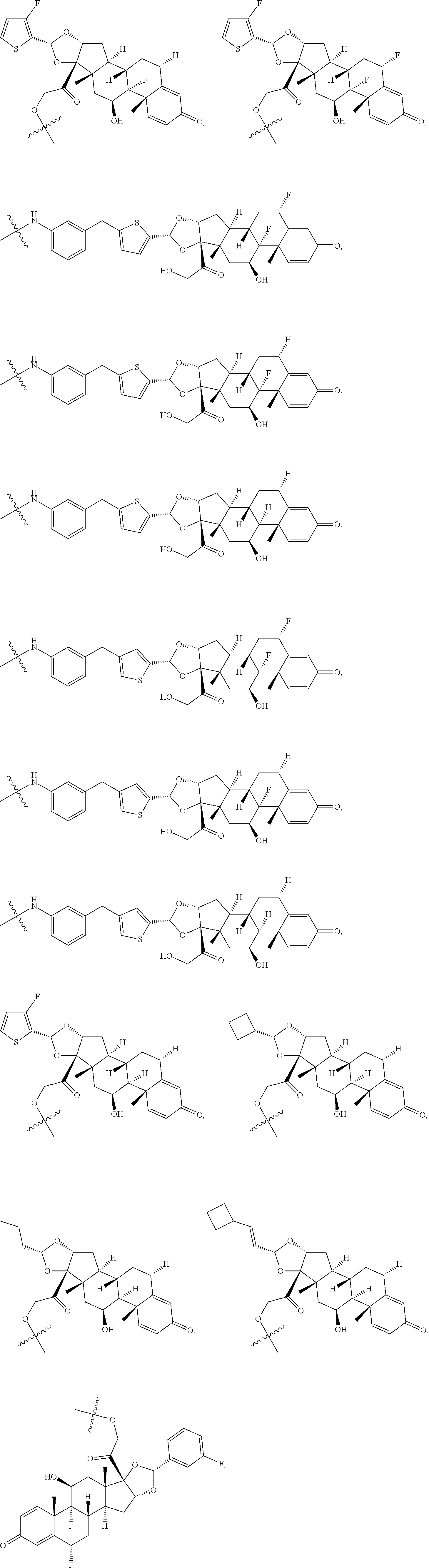

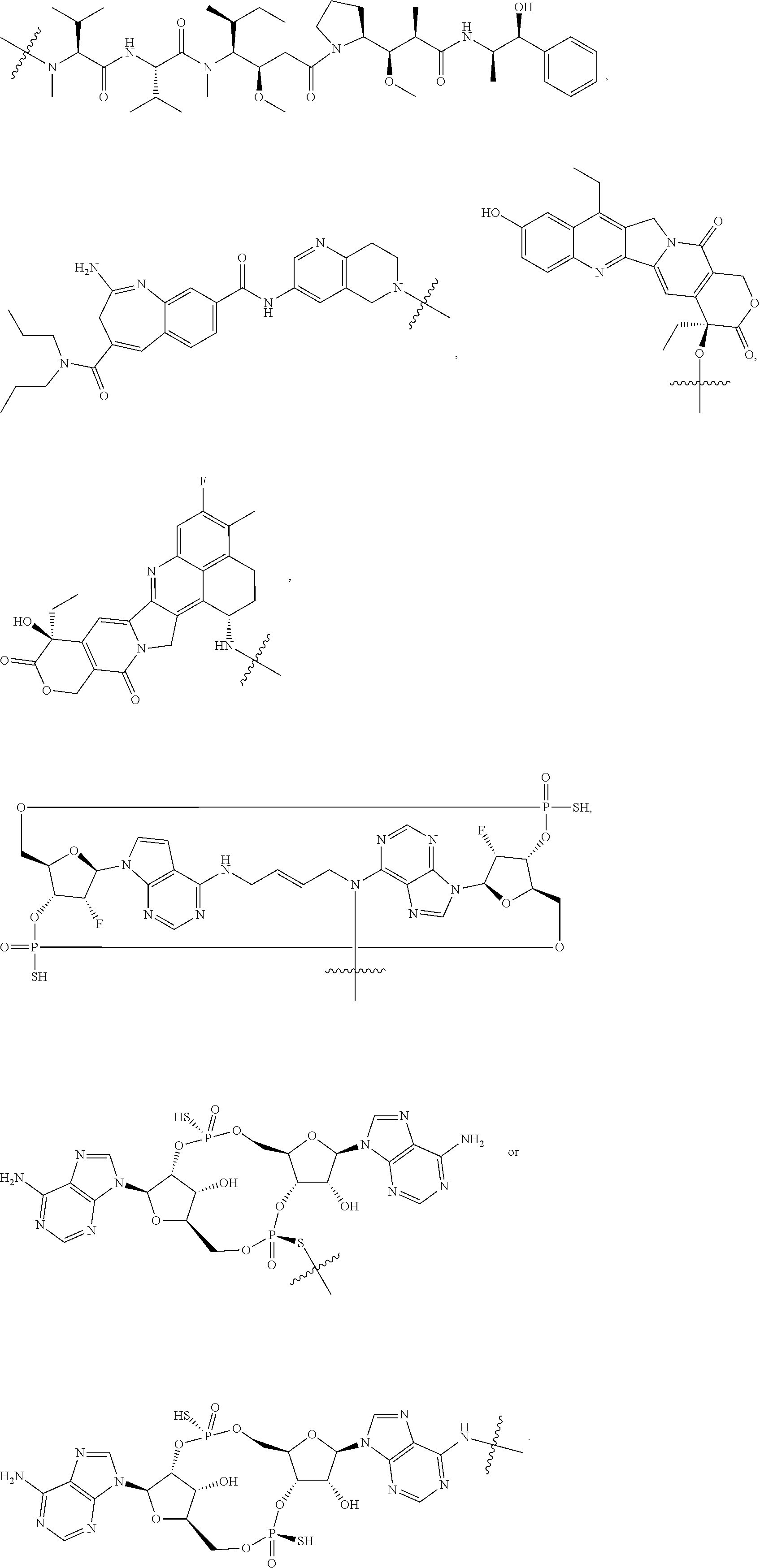

In some embodiments, a payload or R^{1a} has one of the following structures:

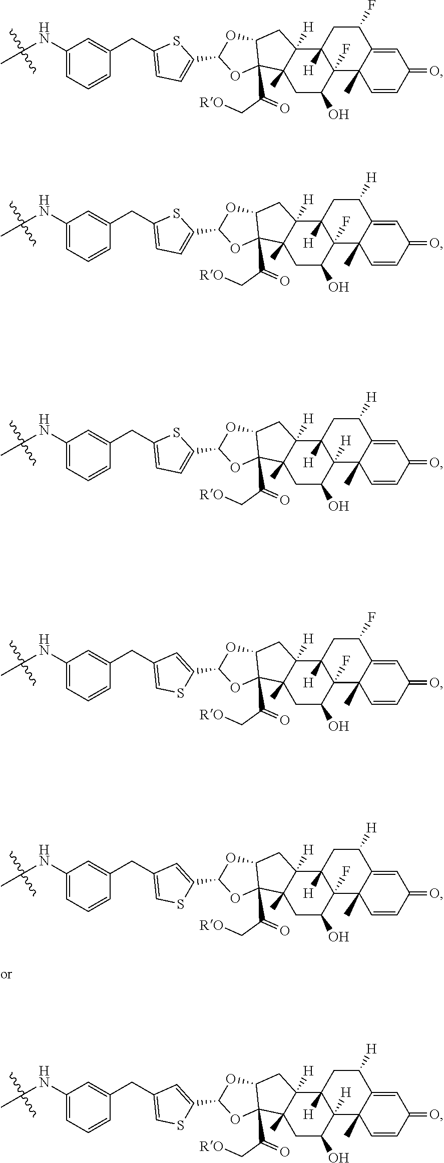

wherein:
R′ is hydrogen or has one of the following structures:

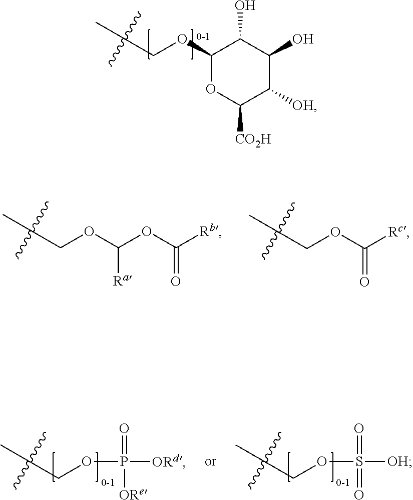

wherein:
R^{a}′ is H or C₁₋₆ alkyl;
R^{b}′ is C₁₋₆ alkyl or C₁₋₆ alkoxy;
R^{c}′ is H, C₁₋₆ alkyl, —CH₂OH, or C₁₋₆ alkoxy;
R^{d}′ is H or C₁₋₆ alkyl; or
R^{e}′ is H or C₁₋₆ alkyl.

In some embodiments, a payload has the following structure:

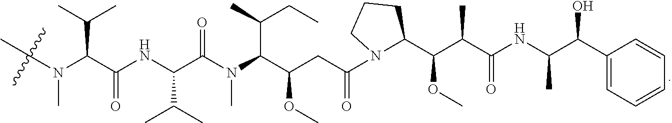 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

In some embodiments, a payload has the following structure:

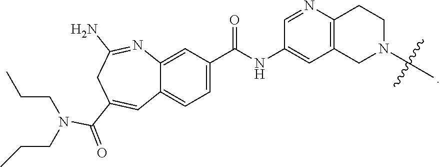 [SMILES: *N1CCc2ncc(NC(=O)c3ccc4c(c3)N=C(N)CC(C(=O)N(CCC)CCC)=C4)cc2C1]

In some embodiments, a payload has the following structure:

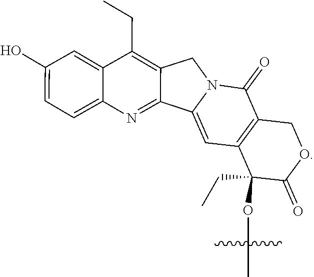 [SMILES: *O[C@]1(CC)C(=O)OCc2c1cc1n(c2=O)Cc2c-1nc1ccc(O)cc1c2CC]

In certain embodiments, a payload has the following structure:

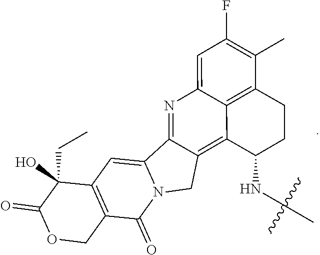 [SMILES: *N[C@H]1CCc2c(C)c(F)cc3nc4c(c1c23)Cn1c-4cc2c(c1=O)COC(=O)[C@]2(O)CC]

In some embodiments, a payload has the following structure:

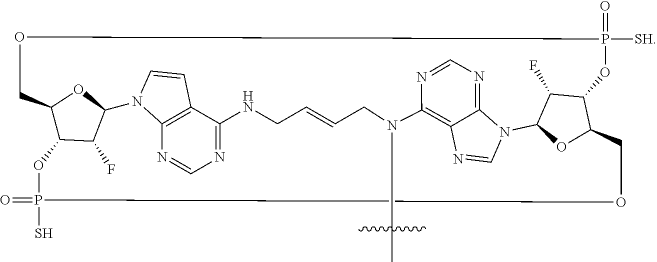 [SMILES: *N1C/C=C/CNc2ncnc3c2ccn3[C@@H]2O[C@@H]3COP(=O)(S)O[C@H]4[C@@H](F)[C@@H](O[C@@H]4COP(=O)(S)O[C@H]3[C@H]2F)n2cnc3c1ncnc32]

In certain embodiments, a payload has the following structure:

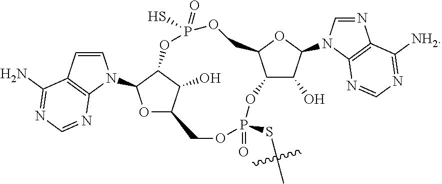 [SMILES: *S[P@@]1(=O)OC[C@H]2O[C@@H](n3ccc4c(N)ncnc43)[C@H](O[P@](=O)(S)OC[C@H]3O[C@@H](n4cnc5c(N)ncnc54)[C@H](O)[C@@H]3O1)[C@@H]2O]

In some embodiments, a payload has the following structure:

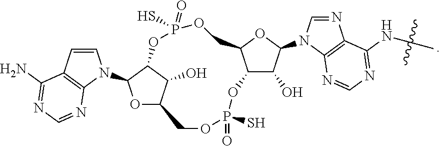 [SMILES: *Nc1ncnc2c1ncn2[C@@H]1O[C@@H]2CO[P@@](=O)(S)O[C@@H]3[C@H](O)[C@@H](CO[P@](=O)(S)O[C@H]2[C@H]1O)O[C@H]3n1ccc2c(N)ncnc21]

In certain embodiments, a payload has the following structure:

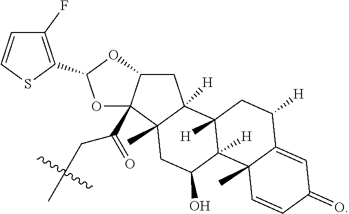

In some embodiments, a payload has the following structure:

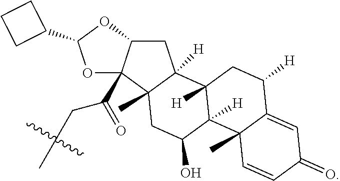

In certain embodiments, a payload has the following structure:

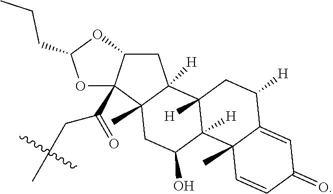

In some embodiments, a payload has the following structure:

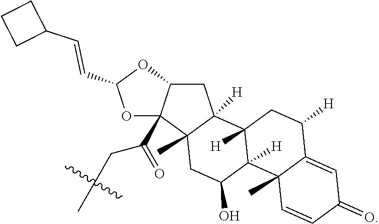

In certain embodiments, a payload has the following structure:

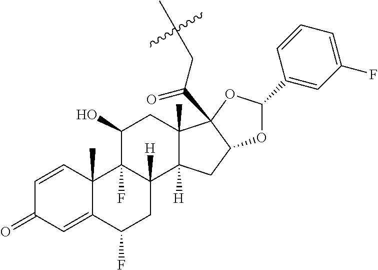

In certain embodiments, a payload has the following structure:

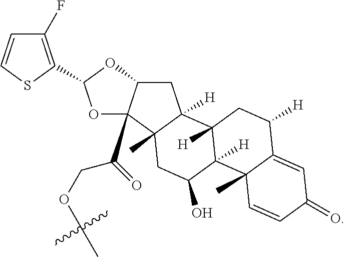

In some embodiments, a payload has the following structure:

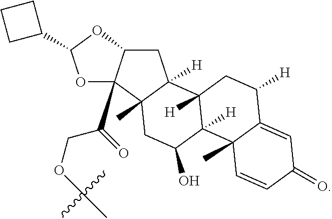

In certain embodiments, a payload has the following structure:

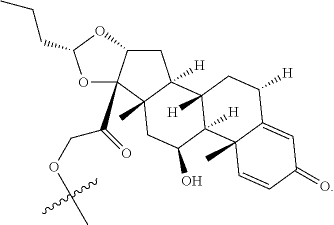

In some embodiments, a payload has the following structure:

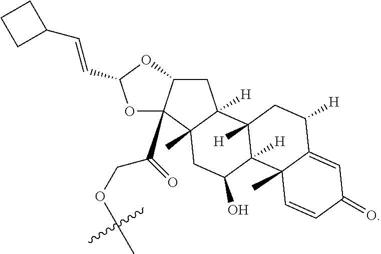

In certain embodiments, a payload has the following structure:

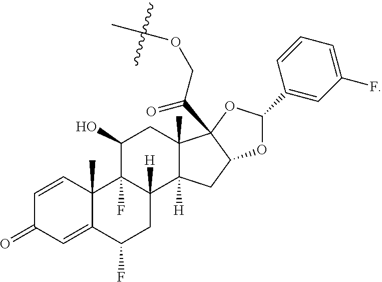

One particular embodiment provides a compound having a structure selected from Table 1 (below), as a stereoisomer, enantiomer or tautomer thereof or a mixture thereof; or a pharmaceutically acceptable salt, solvate or prodrug thereof.

TABLE 1
Representative compounds of Structure (I)

| Compound | |
|---|---|
| No. | Structure |
| I-1 | 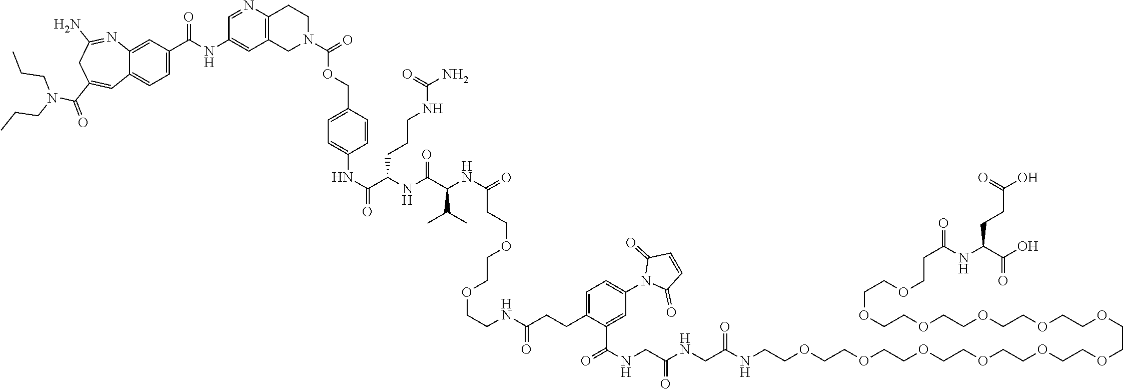 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCOCCOCCNC(=O)CCc5ccc(N6C(=O)C=CC6=O)cc5C(=O)NCC(=O)NCC(=O)NCCOCCOCCOCCOCCOCCOCCOCCOCCOCCOCCOCCOCCC(=O)N[C@@H](CCC(=O)O)C(=O)O)C(C)C)cc3)CC4)cc2N=C(N)C1] |
| I-2 | 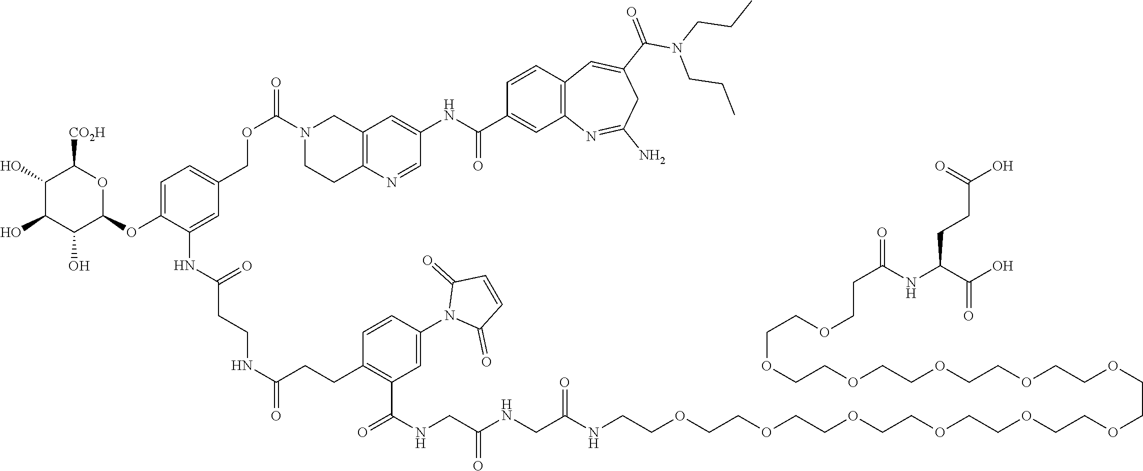 [SMILES: CCCN(CCC)C(=O)C1=Cc2ccc(C(=O)Nc3cnc4c(c3)CN(C(=O)OCc3ccc(O[C@@H]5O[C@H](C(=O)O)[C@@H](O)[C@H](O)[C@H]5O)c(NC(=O)CCNC(=O)CCc5ccc(N6C(=O)C=CC6=O)cc5C(=O)NCC(=O)NCC(=O)NCCOCCOCCOCCOCCOCCOCCOCCOCCOCCOCCOCCOCCC(=O)N[C@@H](CCC(=O)O)C(=O)O)c3)CC4)cc2N=C(N)C1] |
| I-3 | 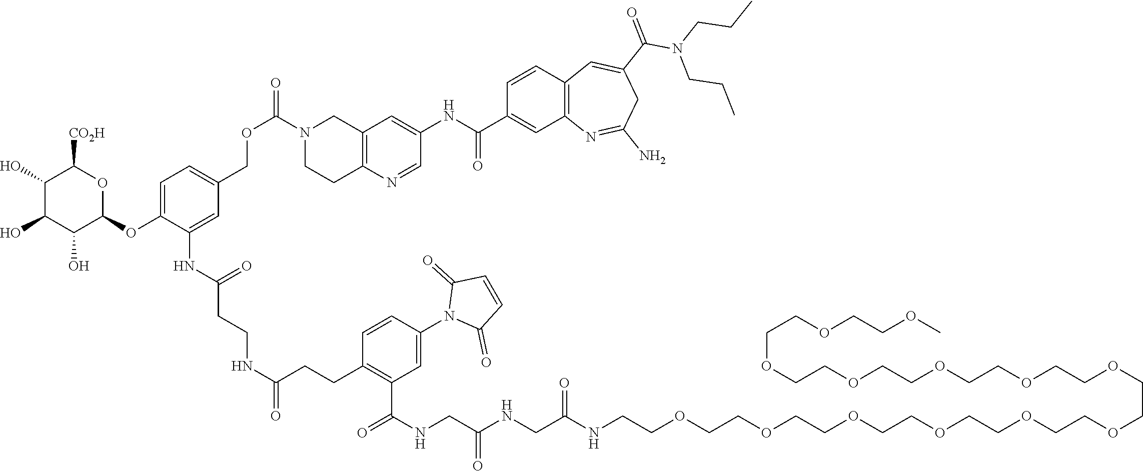 [SMILES: CCCN(CCC)C(=O)C1=Cc2ccc(C(=O)Nc3cnc4c(c3)CN(C(=O)OCc3ccc(O[C@@H]5O[C@H](C(=O)O)[C@@H](O)[C@H](O)[C@H]5O)c(NC(=O)CCNC(=O)CCc5ccc(N6C(=O)C=CC6=O)cc5C(=O)NCC(=O)NCC(=O)NCCOCCOCCOCCOCCOCCOCCOCCOCCOCCOCCOCCOCCOC)c3)CC4)cc2N=C(N)C1] |
| I-4 | 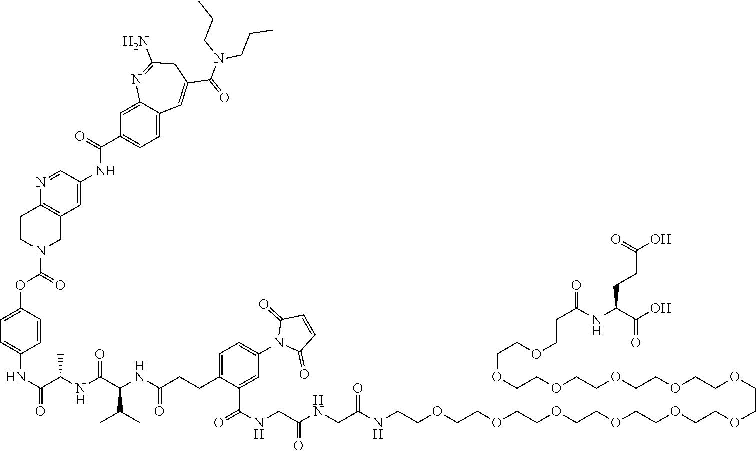 [SMILES: CCCN(CCC)C(=O)C1=Cc2ccc(C(=O)Nc3cnc4c(c3)CN(C(=O)Oc3ccc(NC(=O)[C@H](C)NC(=O)[C@@H](NC(=O)CCc5ccc(N6C(=O)C=CC6=O)cc5C(=O)NCC(=O)NCC(=O)NCCOCCOCCOCCOCCOCCOCCOCCOCCOCCOCCOCCOCCC(=O)N[C@@H](CCC(=O)O)C(=O)O)C(C)C)cc3)CC4)cc2N=C(N)C1] |
| I-5 | 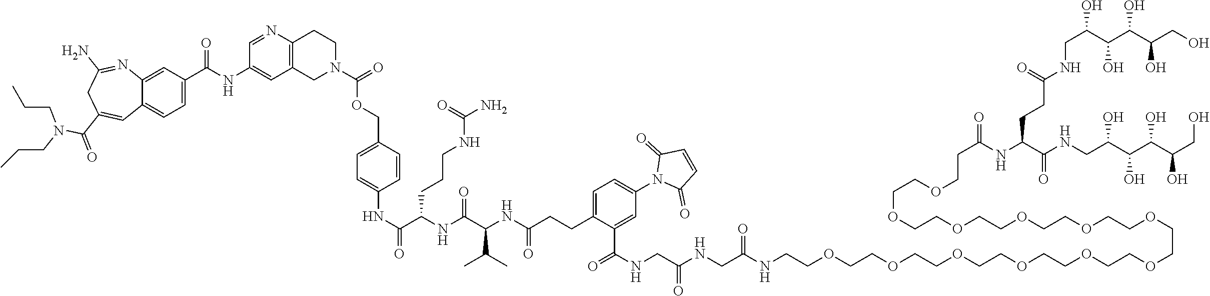 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCc5ccc(N6C(=O)C=CC6=O)cc5C(=O)NCC(=O)NCC(=O)NCCOCCOCCOCCOCCOCCOCCOCCOCCOCCOCCOCCOCCC(=O)N[C@@H](CCC(=O)NC[C@H](O)[C@@H](O)[C@H](O)[C@H](O)CO)C(=O)NC[C@H](O)[C@@H](O)[C@H](O)[C@H](O)CO)C(C)C)cc3)CC4)cc2N=C(N)C1] |
| I-6 | 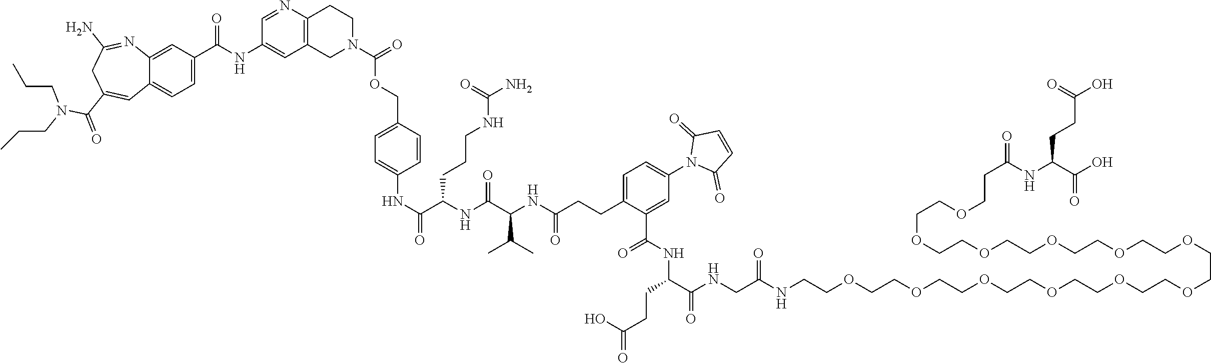 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCc5ccc(N6C(=O)C=CC6=O)cc5C(=O)N[C@@H](CCC(=O)O)C(=O)NCC(=O)NCCOCCOCCOCCOCCOCCOCCOCCOCCOCCOCCOCCOCCC(=O)N[C@@H](CCC(=O)O)C(=O)O)C(C)C)cc3)CC4)cc2N=C(N)C1] |
| I-7 | 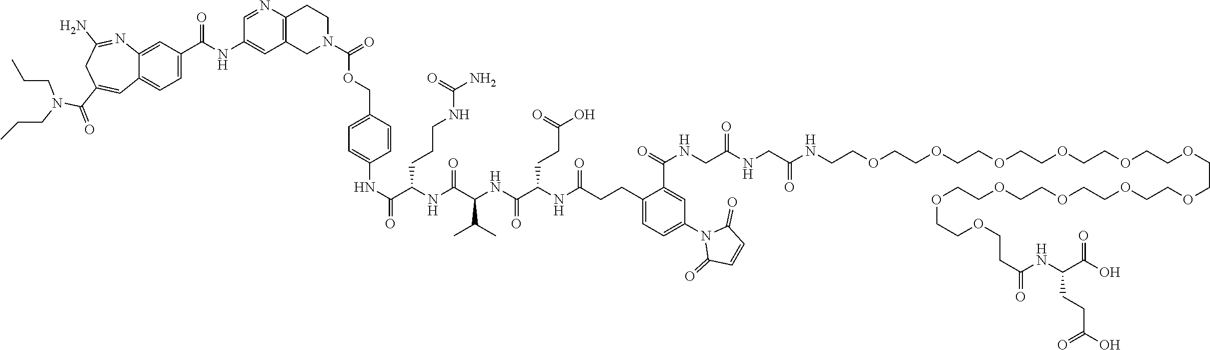 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)[C@H](CCC(=O)O)NC(=O)CCc5ccc(N6C(=O)C=CC6=O)cc5C(=O)NCC(=O)NCC(=O)NCCOCCOCCOCCOCCOCCOCCOCCOCCOCCOCCOCCOCCC(=O)N[C@@H](CCC(=O)O)C(=O)O)C(C)C)cc3)CC4)cc2N=C(N)C1] |
| I-8 | 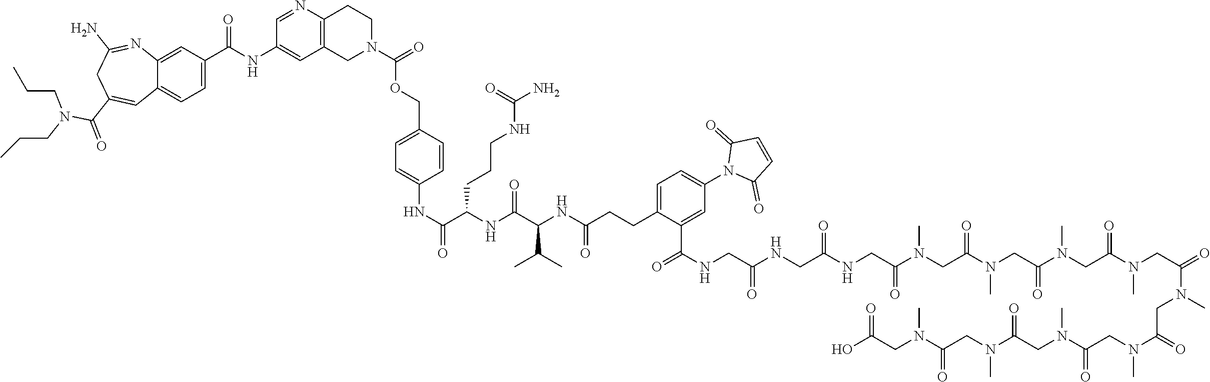 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCc5ccc(N6C(=O)C=CC6=O)cc5C(=O)NCC(=O)NCC(=O)NCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C(C)C)cc3)CC4)cc2N=C(N)C1] |
| I-9 | 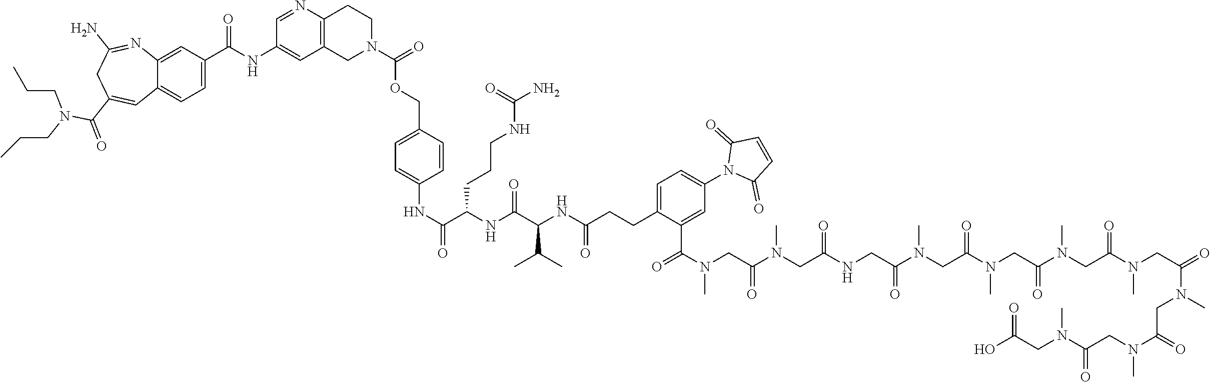 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCc5ccc(N6C(=O)C=CC6=O)cc5C(=O)N(C)CC(=O)N(C)CC(=O)NCC(=O)N(C)CC(=O)N(C)CC(=O)N(C)CC(=O)N(C)CC(=O)N(C)CC(=O)N(C)CC(=O)N(C)CC(=O)O)C(C)C)cc3)CC4)cc2N=C(N)C1] |
| I-10 | 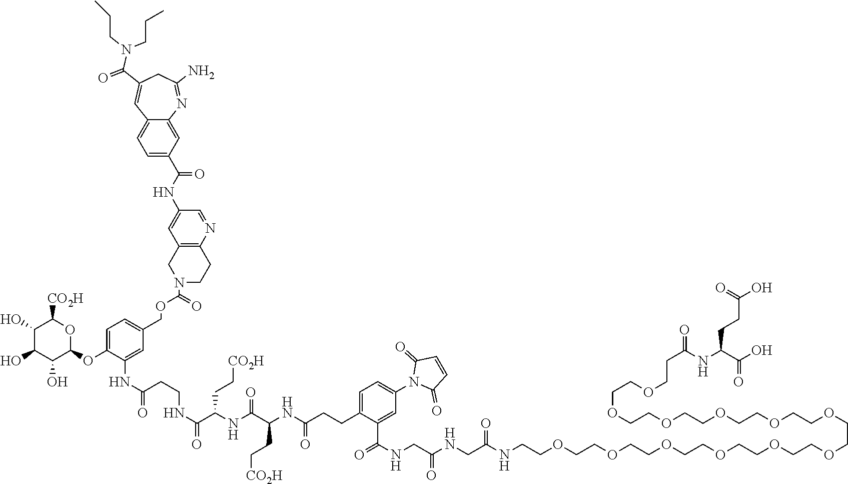 [SMILES: CCCN(CCC)C(=O)C1=Cc2ccc(C(=O)Nc3cnc4c(c3)CN(C(=O)OCc3ccc(O[C@@H]5O[C@H](C(=O)O)[C@@H](O)[C@H](O)[C@H]5O)c(NC(=O)CCNC(=O)[C@H](CCC(=O)O)NC(=O)[C@H](CCC(=O)O)NC(=O)CCc5ccc(N6C(=O)C=CC6=O)cc5C(=O)NCC(=O)NCC(=O)NCCOCCOCCOCCOCCOCCOCCOCCOCCOCCOCCOCCOCCC(=O)N[C@@H](CCC(=O)O)C(=O)O)c3)CC4)cc2N=C(N)C1] |
| I-11 | 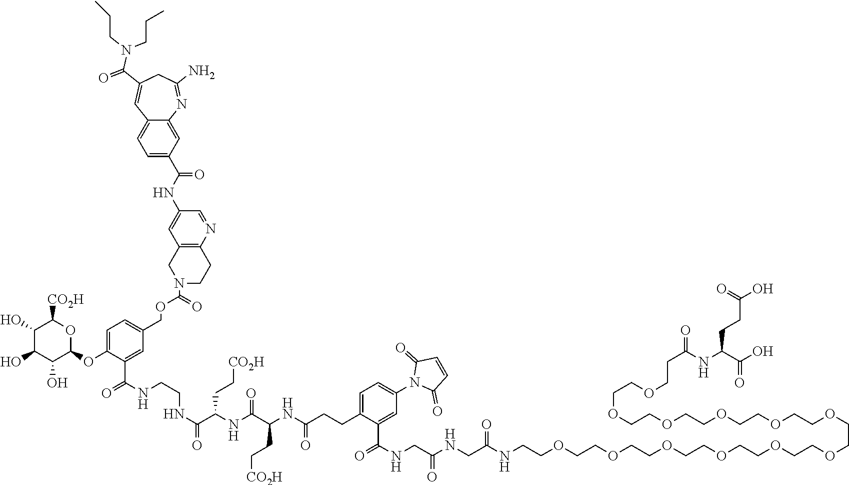 [SMILES: CCCN(CCC)C(=O)C1=Cc2ccc(C(=O)Nc3cnc4c(c3)CN(C(=O)OCc3ccc(O[C@@H]5O[C@H](C(=O)O)[C@@H](O)[C@H](O)[C@H]5O)c(C(=O)NCCNC(=O)[C@H](CCC(=O)O)NC(=O)[C@H](CCC(=O)O)NC(=O)CCc5ccc(N6C(=O)C=CC6=O)cc5C(=O)NCC(=O)NCC(=O)NCCOCCOCCOCCOCCOCCOCCOCCOCCOCCOCCOCCOCCC(=O)N[C@@H](CCC(=O)O)C(=O)O)c3)CC4)cc2N=C(N)C1] |
| I-12 | 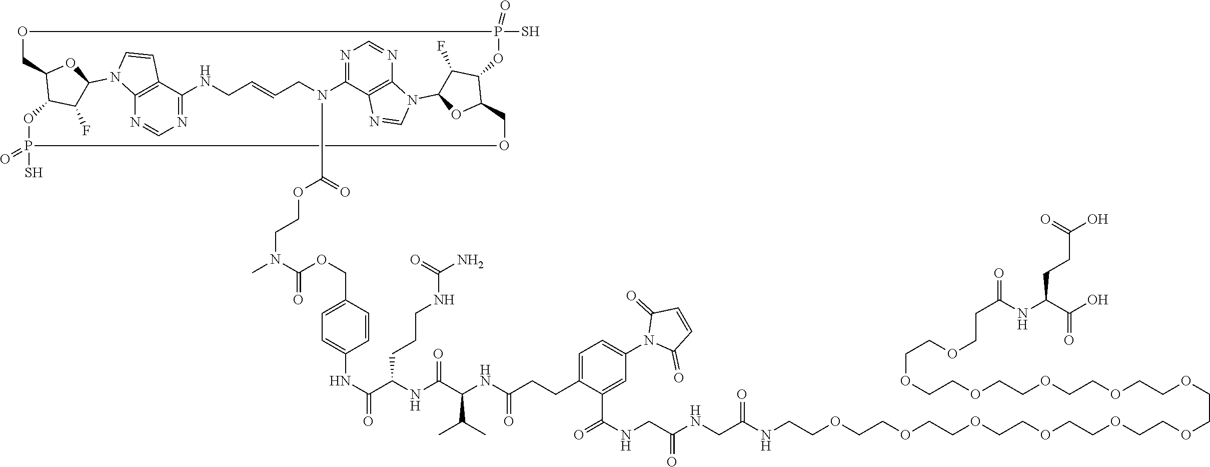 [SMILES: CC(C)[C@H](NC(=O)CCc1ccc(N2C(=O)C=CC2=O)cc1C(=O)NCC(=O)NCC(=O)NCCOCCOCCOCCOCCOCCOCCOCCOCCOCCOCCOCCOCCC(=O)N[C@@H](CCC(=O)O)C(=O)O)C(=O)N[C@@H](CCCNC(N)=O)C(=O)Nc1ccc(COC(=O)N(C)CCOC(=O)N2C/C=C/CNc3ncnc4c3ccn4[C@@H]3O[C@@H]4COP(=O)(S)O[C@H]5[C@@H](F)[C@@H](O[C@@H]5COP(=O)(S)O[C@H]4[C@H]3F)n3cnc4c2ncnc43)cc1] |
| I-13 | 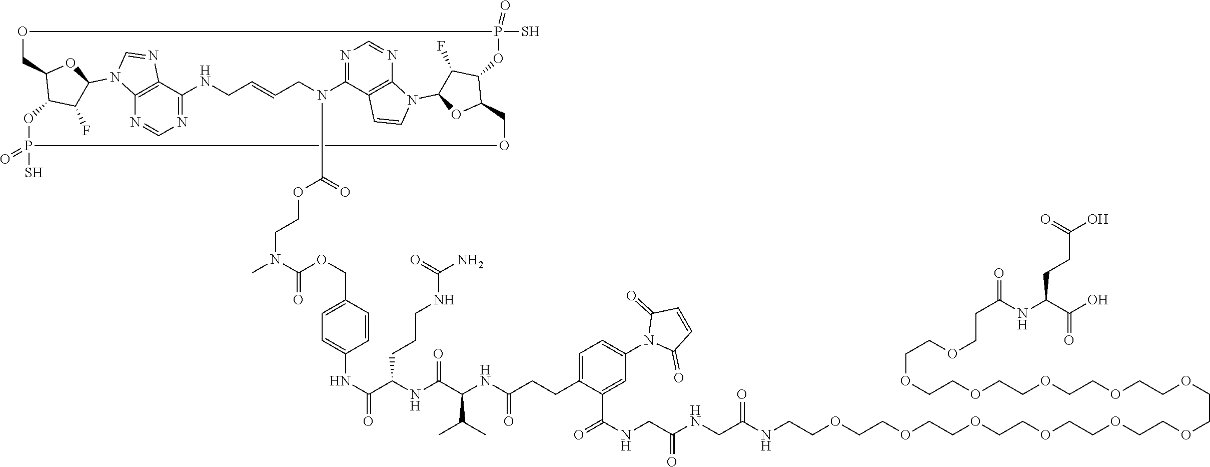 [SMILES: CC(C)[C@H](NC(=O)CCc1ccc(N2C(=O)C=CC2=O)cc1C(=O)NCC(=O)NCC(=O)NCCOCCOCCOCCOCCOCCOCCOCCOCCOCCOCCOCCOCCC(=O)N[C@@H](CCC(=O)O)C(=O)O)C(=O)N[C@@H](CCCNC(N)=O)C(=O)Nc1ccc(COC(=O)N(C)CCOC(=O)N2C/C=C/CNc3ncnc4c3ncn4[C@@H]3O[C@@H]4COP(=O)(S)O[C@H]5[C@@H](F)[C@@H](O[C@@H]5COP(=O)(S)O[C@H]4[C@H]3F)n3ccc4c2ncnc43)cc1] |
| I-14 | 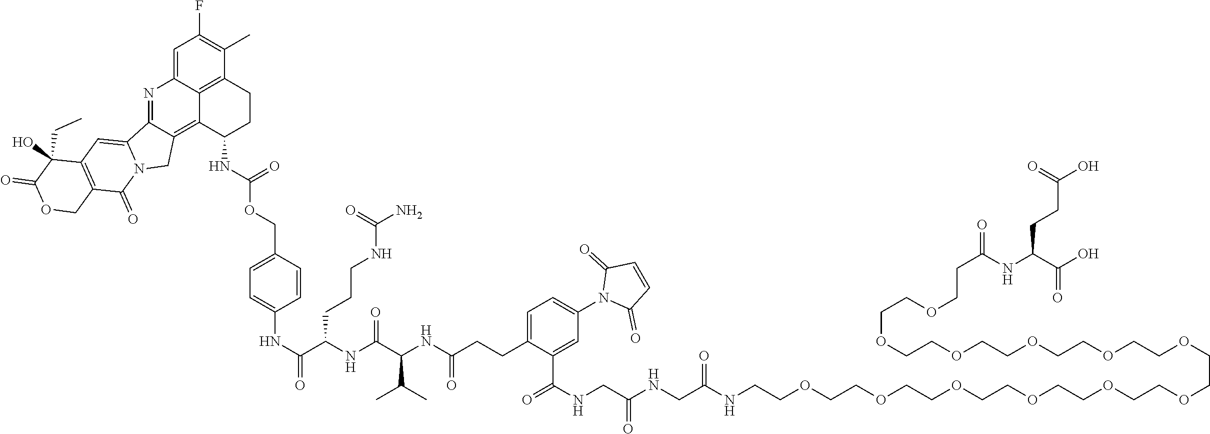 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)CCc2ccc(N4C(=O)C=CC4=O)cc2C(=O)NCC(=O)NCC(=O)NCCOCCOCCOCCOCCOCCOCCOCCOCCOCCOCCOCCOCCC(=O)N[C@@H](CCC(=O)O)C(=O)O)C(C)C)cc1)CC3] |
| I-15 | 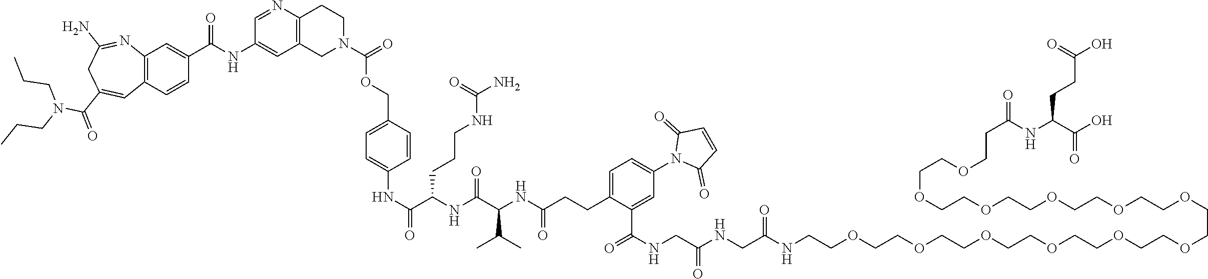 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCc5ccc(N6C(=O)C=CC6=O)cc5C(=O)NCC(=O)NCC(=O)NCCOCCOCCOCCOCCOCCOCCOCCOCCOCCOCCOCCOCCC(=O)N[C@@H](CCC(=O)O)C(=O)O)C(C)C)cc3)CC4)cc2N=C(N)C1] |
| I-16 | 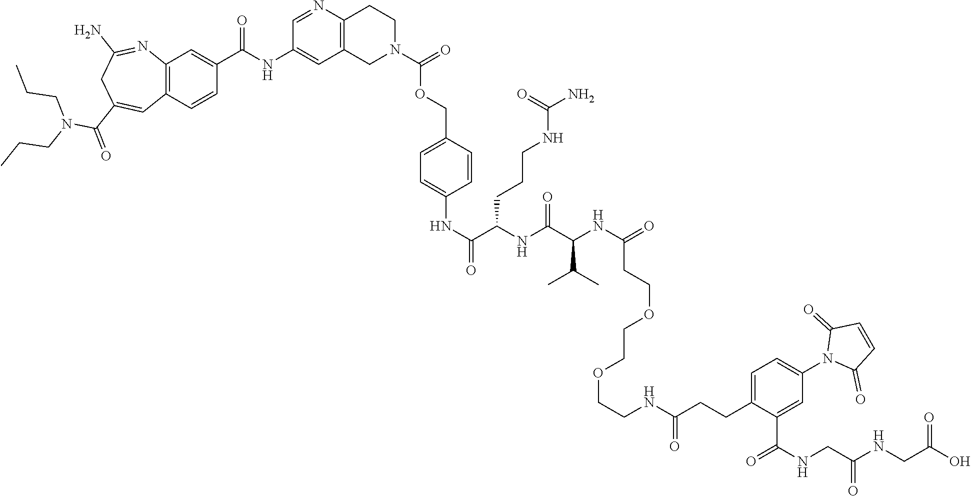 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCOCCOCCNC(=O)CCc5ccc(N6C(=O)C=CC6=O)cc5C(=O)NCC(=O)NCC(=O)O)C(C)C)cc3)CC4)cc2N=C(N)C1] |
| I-17 | 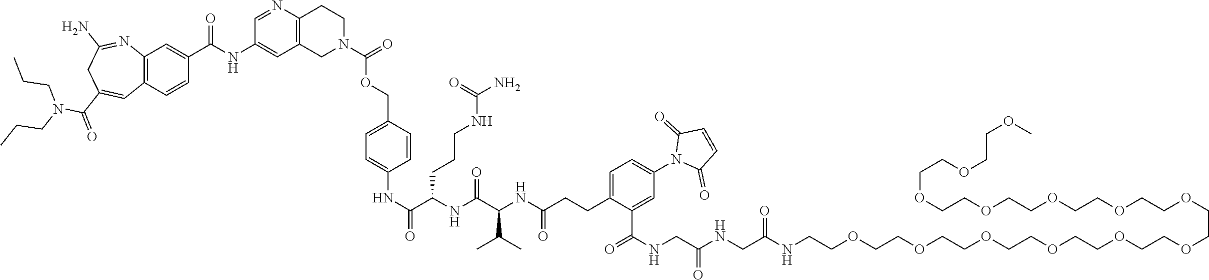 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCc5ccc(N6C(=O)C=CC6=O)cc5C(=O)NCC(=O)NCC(=O)NCCOCCOCCOCCOCCOCCOCCOCCOCCOCCOCCOCCOCCOC)C(C)C)cc3)CC4)cc2N=C(N)C1] |
| I-18 | 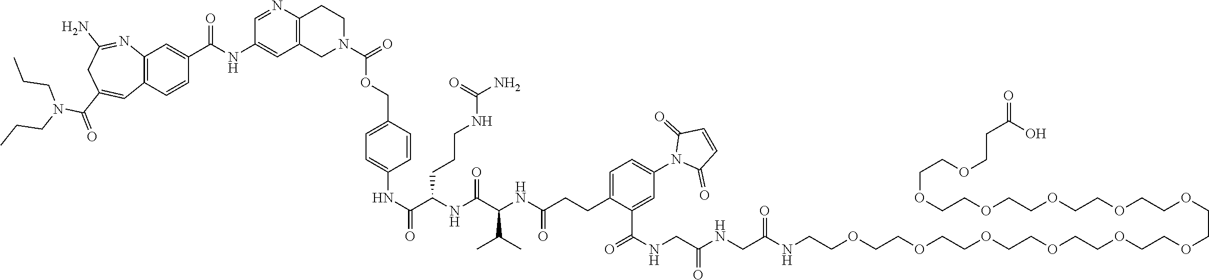 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCc5ccc(N6C(=O)C=CC6=O)cc5C(=O)NCC(=O)NCC(=O)NCCOCCOCCOCCOCCOCCOCCOCCOCCOCCOCCOCCOCCC(=O)O)C(C)C)cc3)CC4)cc2N=C(N)C1] |
| I-19 | 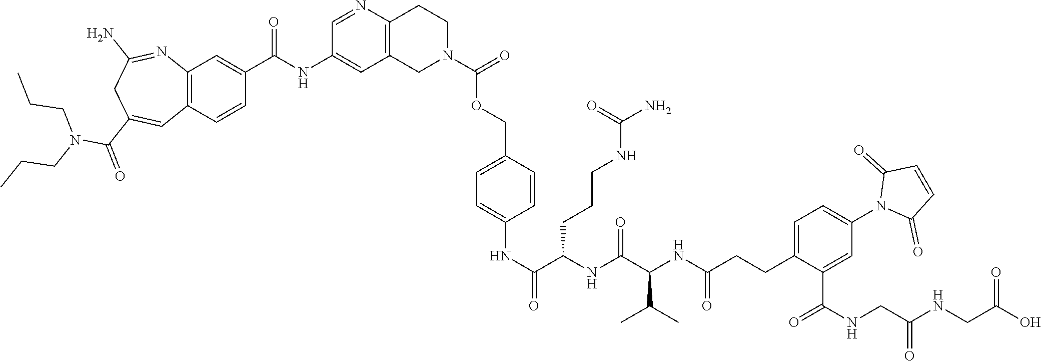 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCc5ccc(N6C(=O)C=CC6=O)cc5C(=O)NCC(=O)NCC(=O)O)C(C)C)cc3)CC4)cc2N=C(N)C1] |
| I-20 | 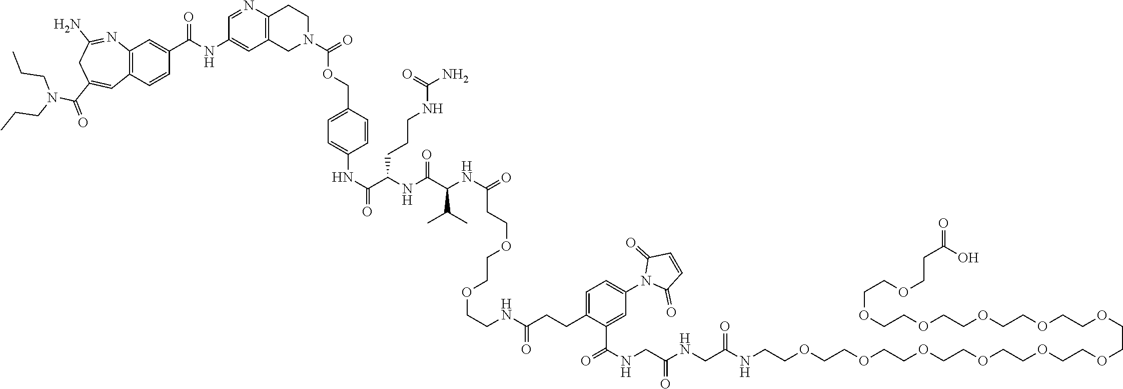 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCOCCOCCNC(=O)CCc5ccc(N6C(=O)C=CC6=O)cc5C(=O)NCC(=O)NCC(=O)NCCOCCOCCOCCOCCOCCOCCOCCOCCOCCOCCOCCOCCC(=O)O)C(C)C)cc3)CC4)cc2N=C(N)C1] |
| I-21 | 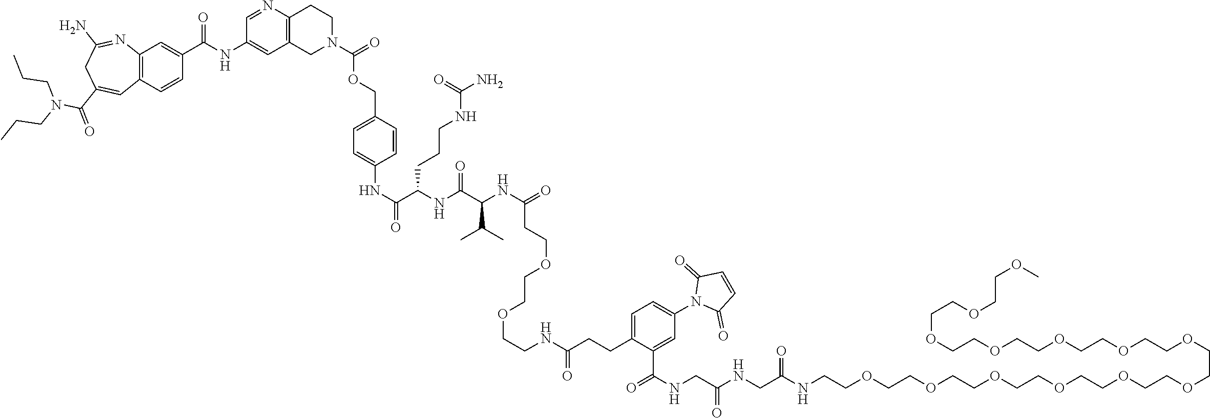 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCOCCOCCNC(=O)CCc5ccc(N6C(=O)C=CC6=O)cc5C(=O)NCC(=O)NCC(=O)NCCOCCOCCOCCOCCOCCOCCOCCOCCOCCOCCOCCOCCOC)C(C)C)cc3)CC4)cc2N=C(N)C1] |
| I-22 | 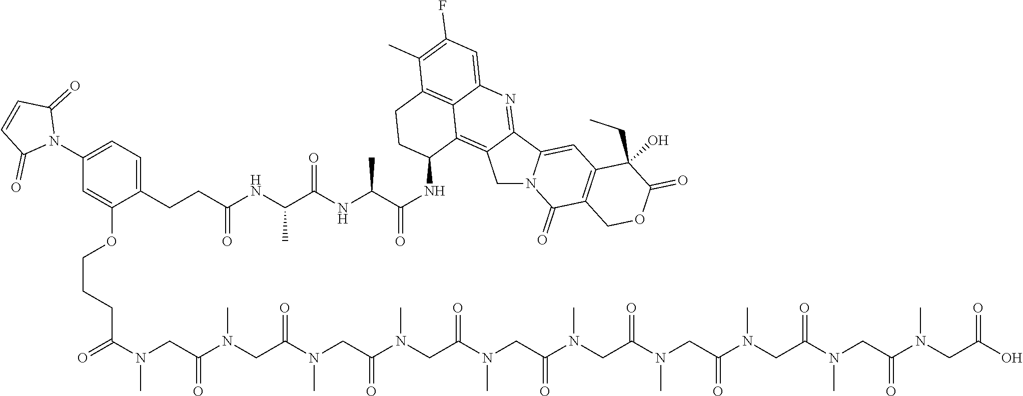 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@H](C)NC(=O)CCc1ccc(N2C(=O)C=CC2=O)cc1OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)CC3] |
| I-23 | 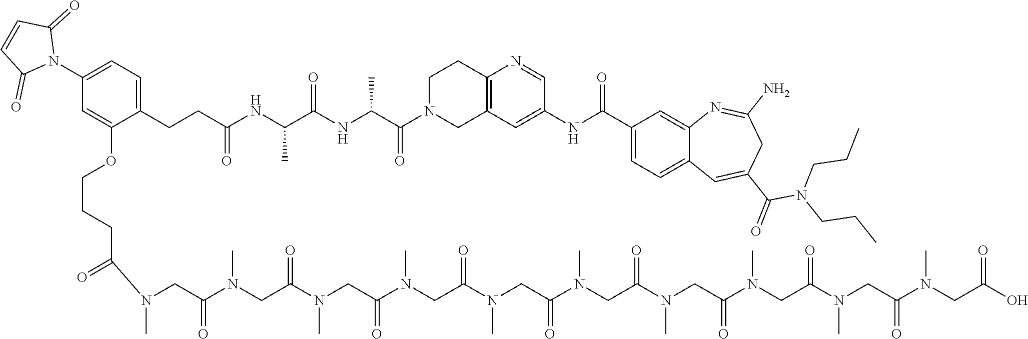 [SMILES: CCCN(CCC)C(=O)C1=Cc2ccc(C(=O)Nc3cnc4c(c3)CN(C(=O)[C@@H](C)NC(=O)[C@H](C)NC(=O)CCc3ccc(N5C(=O)C=CC5=O)cc3OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)CC4)cc2N=C(N)C1] |
| I-24 | 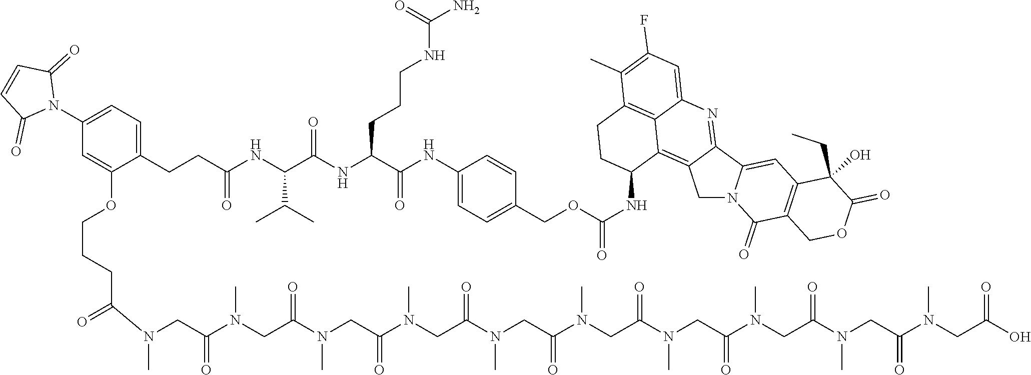 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)CCc2ccc(N4C(=O)C=CC4=O)cc2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C(C)C)cc1)CC3] |
| I-25 | 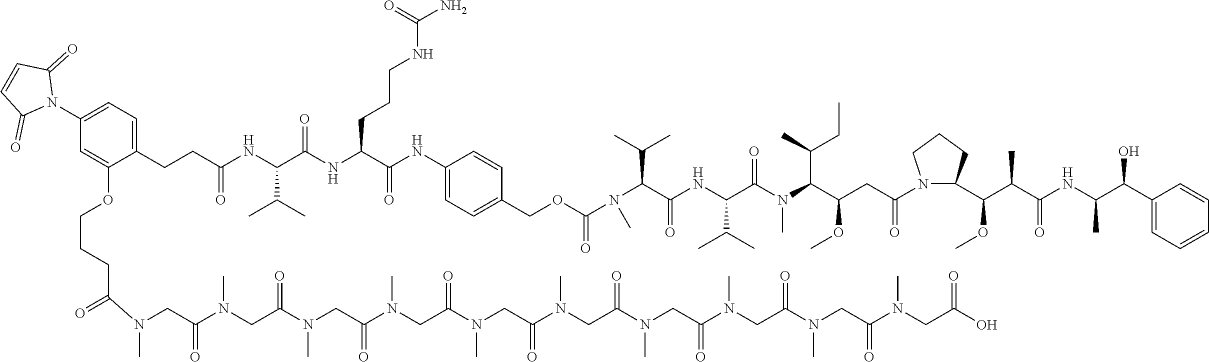 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCc2ccc(N3C(=O)C=CC3=O)cc2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C(C)C)cc1)C(C)C] |
| I-26 | 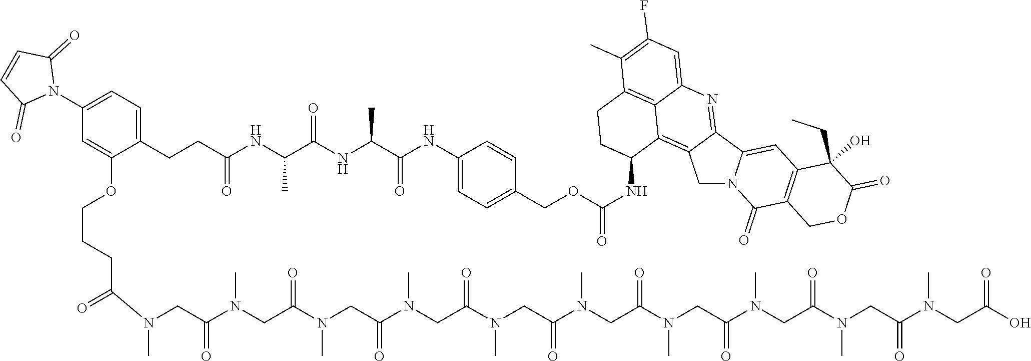 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@H](C)NC(=O)CCc2ccc(N4C(=O)C=CC4=O)cc2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)cc1)CC3] |
| I-27 | 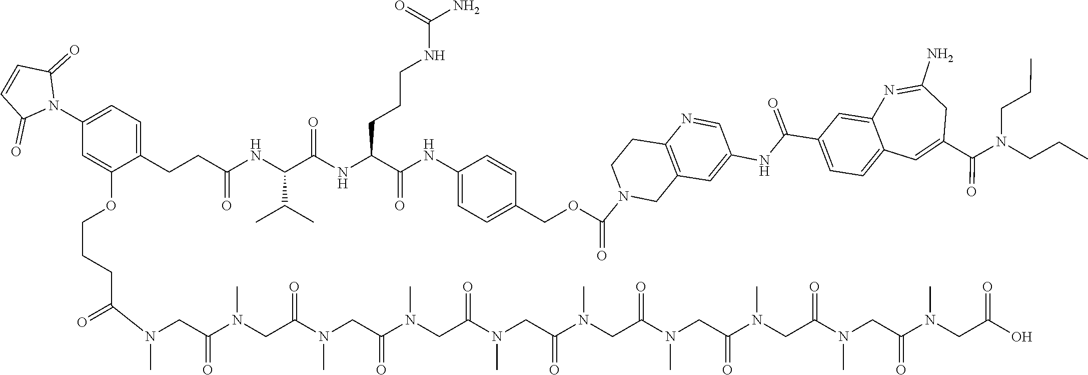 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCc5ccc(N6C(=O)C=CC6=O)cc5OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)C(C)C)cc3)CC4)cc2N=C(N)C1] |
| I-28 | 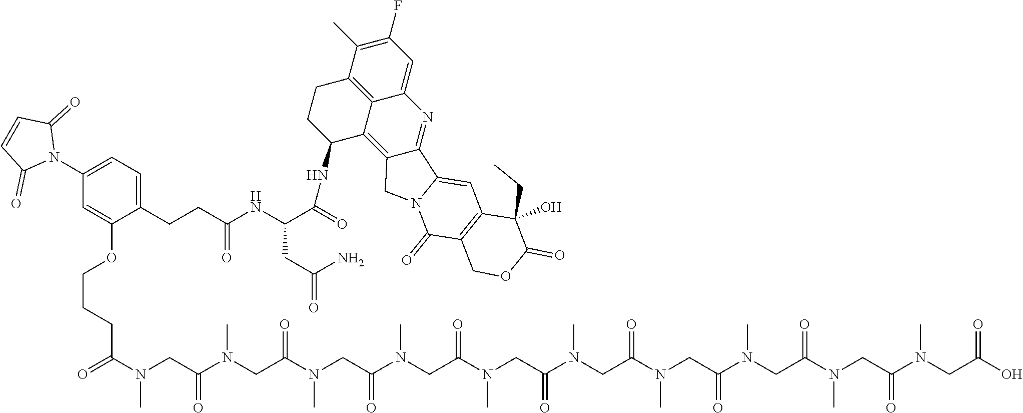 [SMILES: CC[C@@]1(O)C(=O)OCc2c1cc1n(c2=O)Cc2c-1nc1cc(F)c(C)c3c1c2[C@@H](NC(=O)[C@H](CC(N)=O)NC(=O)CCc1ccc(N2C(=O)C=CC2=O)cc1OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)CC3] |
| I-29 | 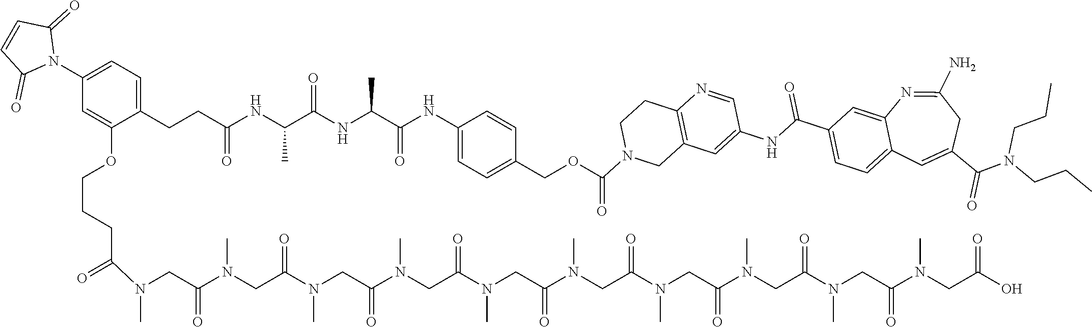 [SMILES: CCCN(CCC)C(=O)C1=Cc2ccc(C(=O)Nc3cnc4c(c3)CN(C(=O)OCc3ccc(NC(=O)[C@H](C)NC(=O)[C@H](C)NC(=O)CCc5ccc(N6C(=O)C=CC6=O)cc5OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)cc3)CC4)cc2N=C(N)C1] |
| I-30 | 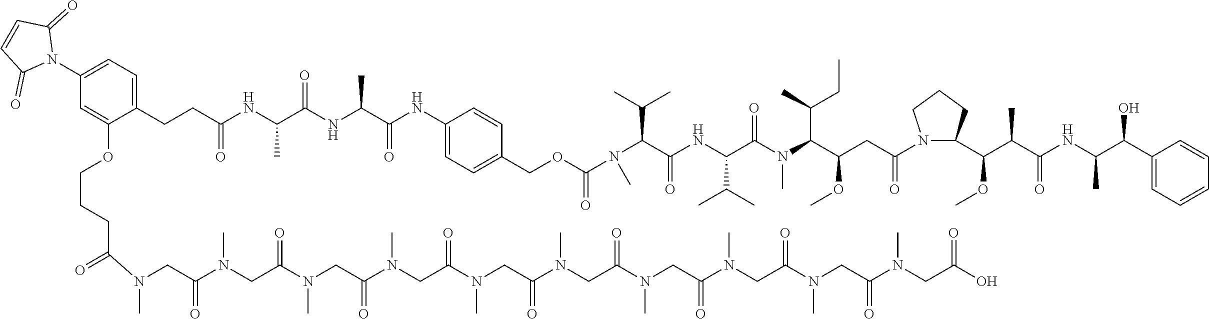 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@H](C)NC(=O)CCc2ccc(N3C(=O)C=CC3=O)cc2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)cc1)C(C)C] |
| I-31 | 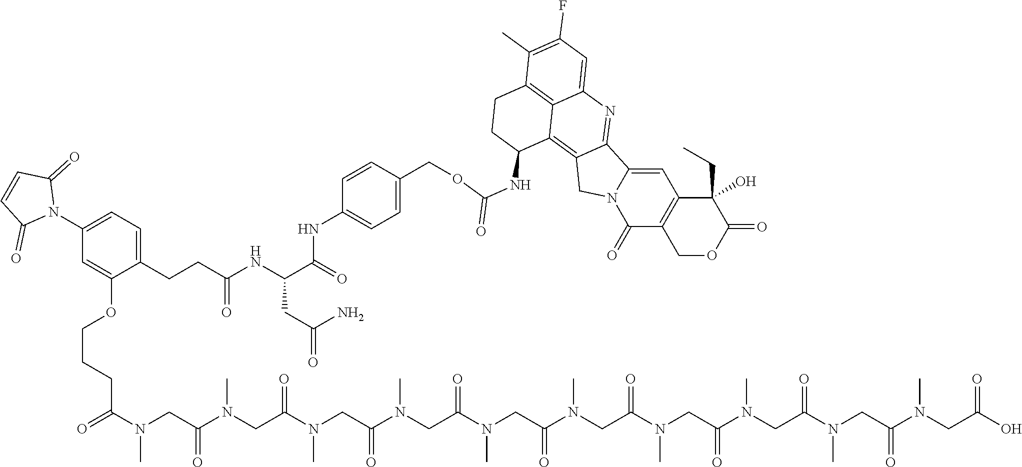 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CC(N)=O)NC(=O)CCc2ccc(N4C(=O)C=CC4=O)cc2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)cc1)CC3] |
| I-32 | 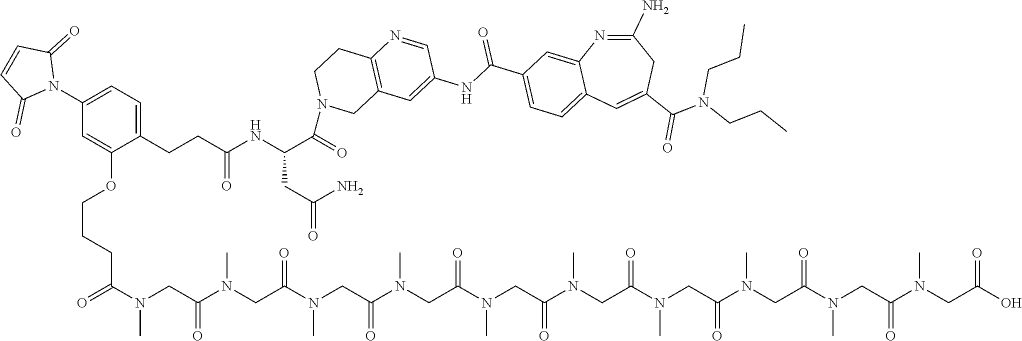 [SMILES: CCCN(CCC)C(=O)C1=Cc2ccc(C(=O)Nc3cnc4c(c3)CN(C(=O)[C@H](CC(N)=O)NC(=O)CCc3ccc(N5C(=O)C=CC5=O)cc3OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)CC4)cc2N=C(N)C1] |
| I-33 | 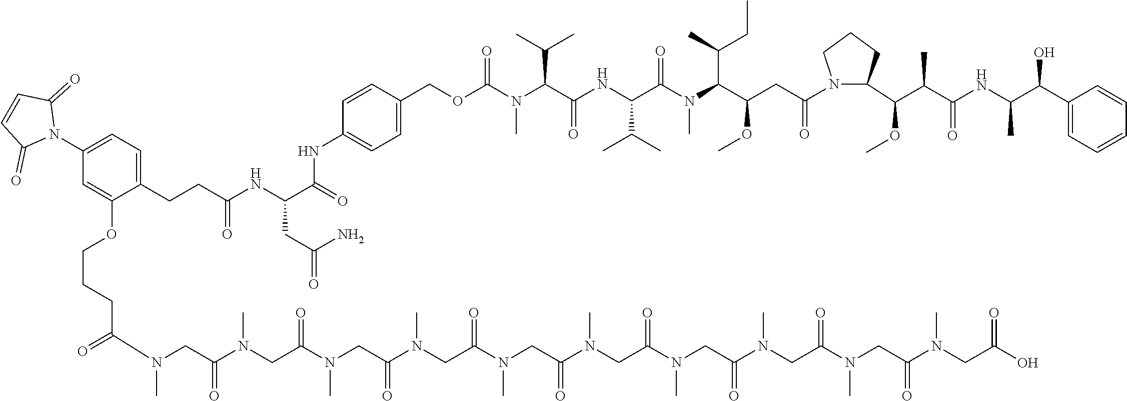 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CC(N)=O)NC(=O)CCc2ccc(N3C(=O)C=CC3=O)cc2OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)cc1)C(C)C] |
| I-34 | 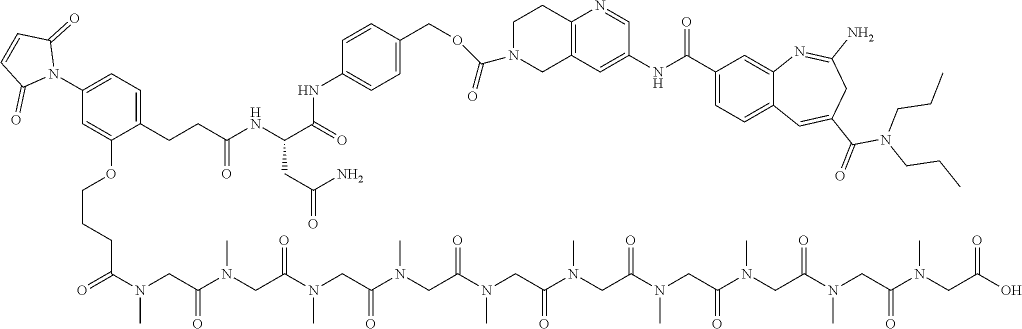 [SMILES: CCCN(CCC)C(=O)C1=Cc2ccc(C(=O)Nc3cnc4c(c3)CN(C(=O)OCc3ccc(NC(=O)[C@H](CC(N)=O)NC(=O)CCc5ccc(N6C(=O)C=CC6=O)cc5OCCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)cc3)CC4)cc2N=C(N)C1] |
| I-35 | 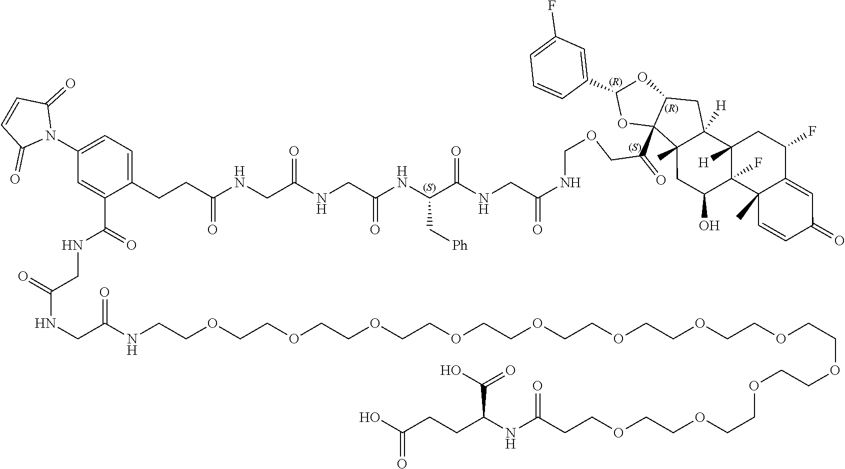 |
| I-36 | 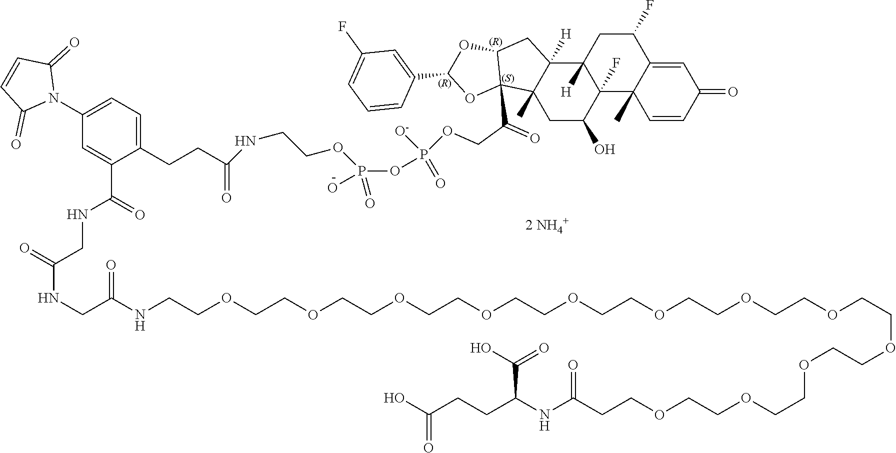 |
| I-37 | 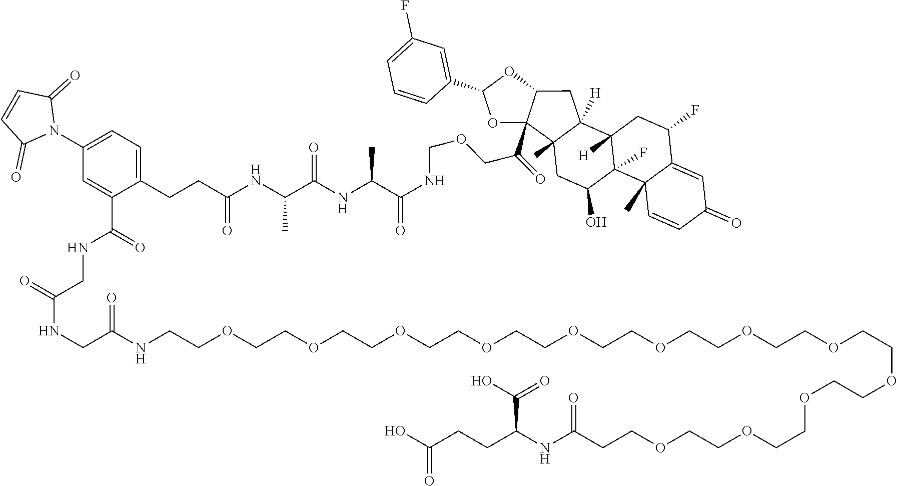 |
| I-38 | 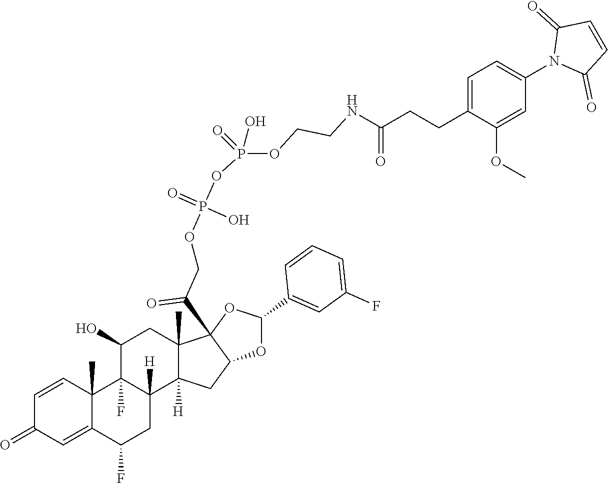 |
| I-39 | 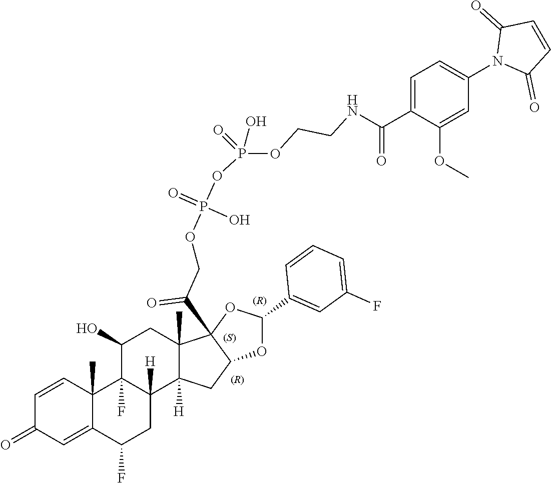 |
| I-40 | 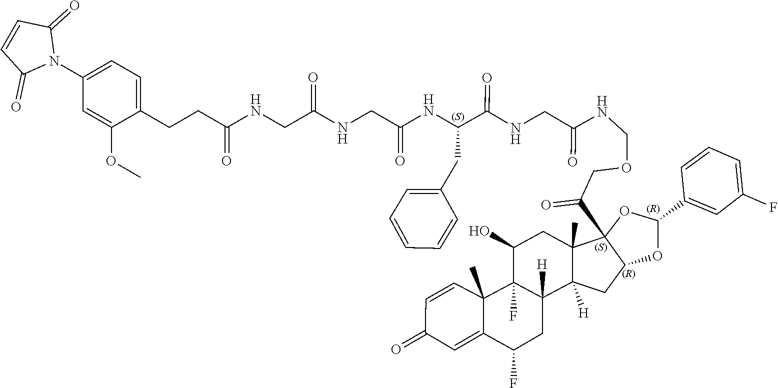 |
| I-41 | 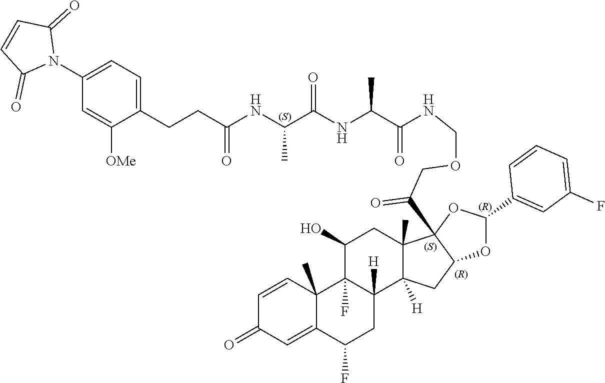 |
| I-42 | 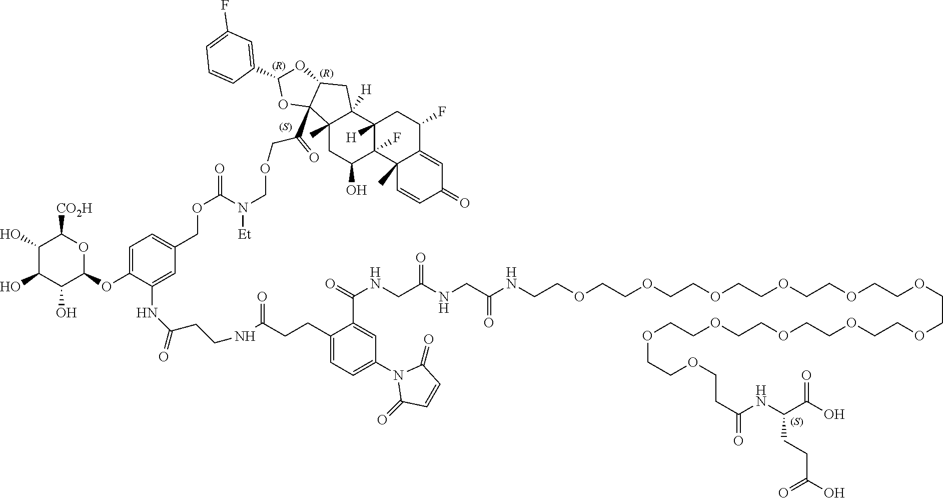 |
| I-43 | 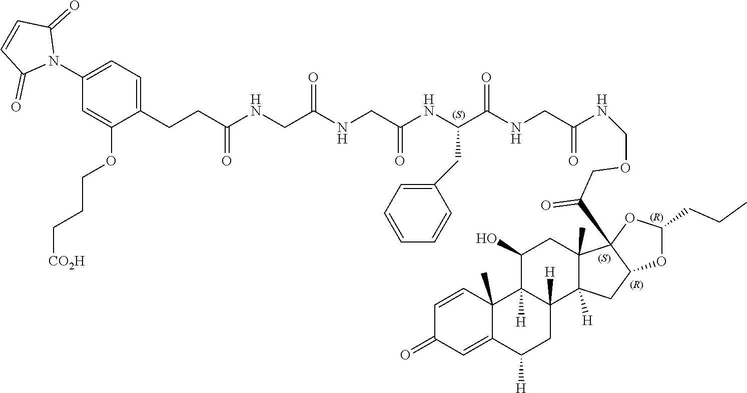 |
| I-44 | 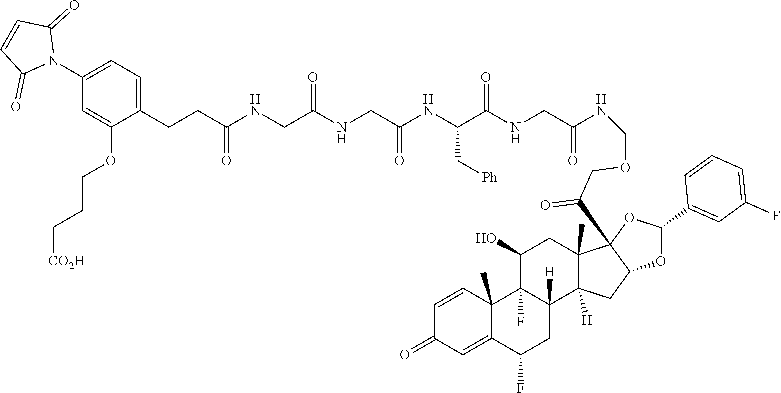 |
| I-45 | 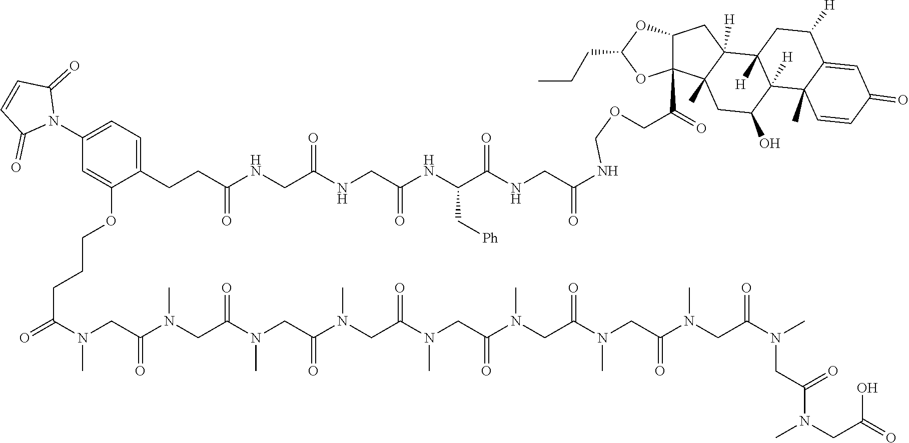 |
| I-46 | 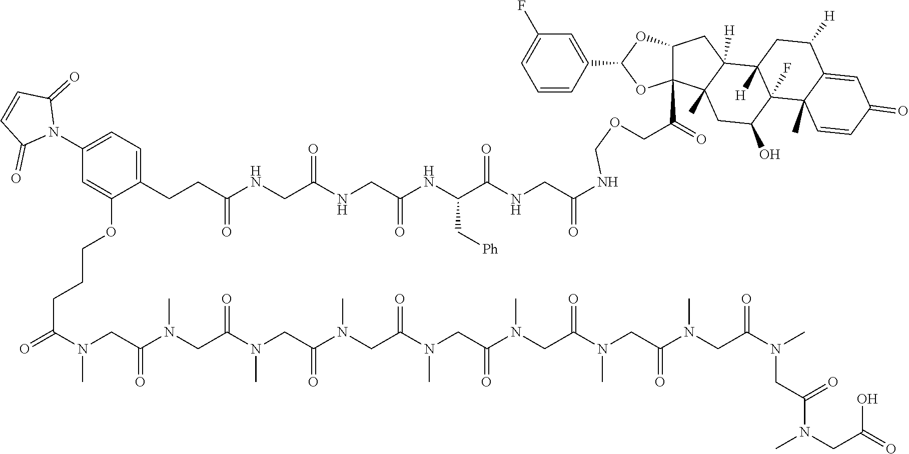 |
| I-47 | 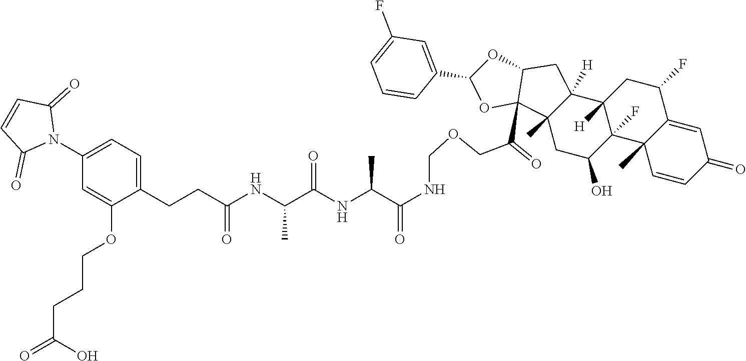 |
| I-48 | 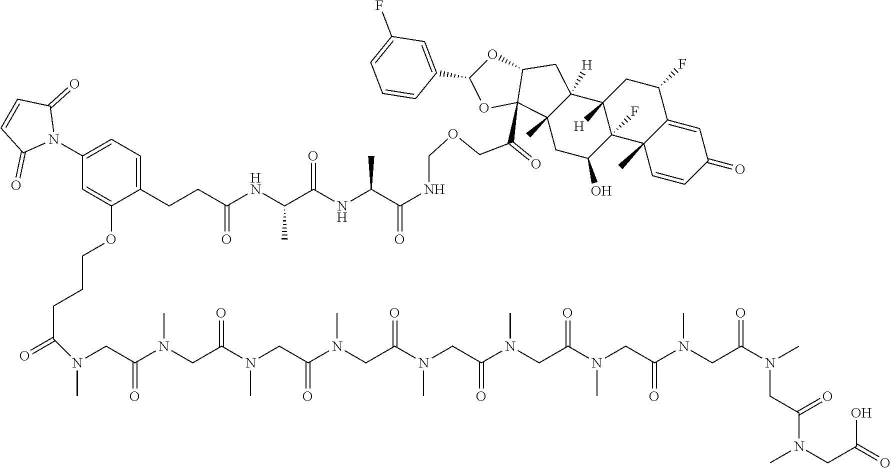 |
| I-49 | 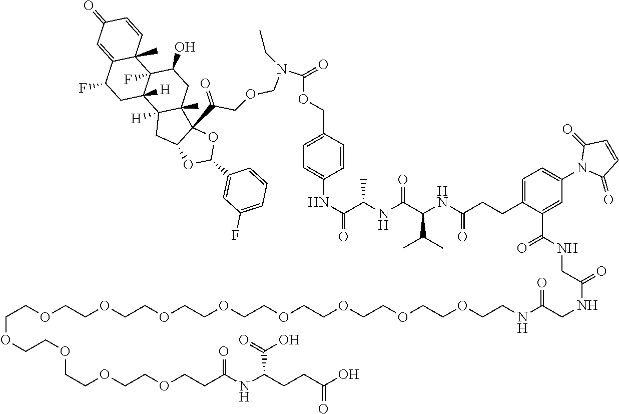 |
| I-50 | 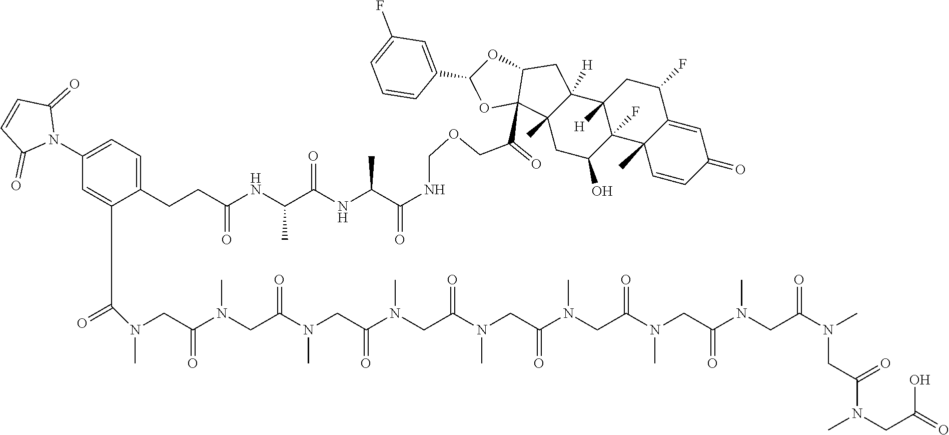 |
| I-51 | 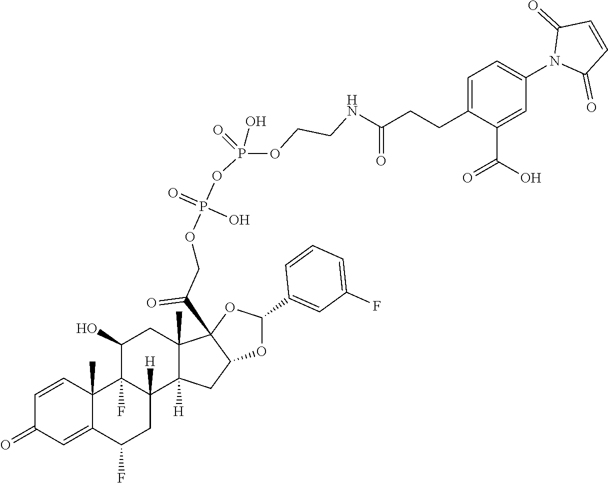 |
| I-52 | 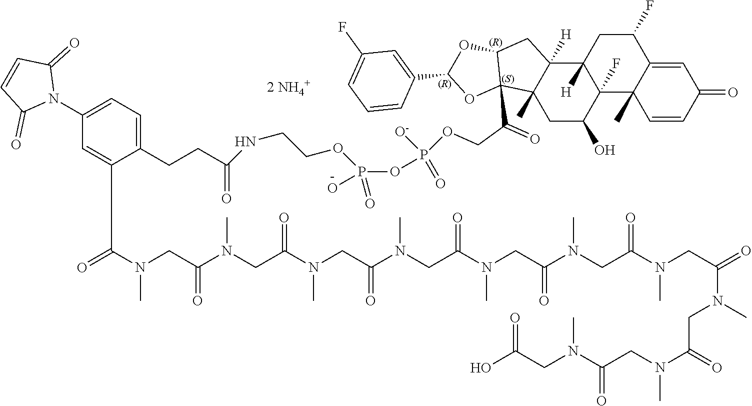 |
| I-53 | 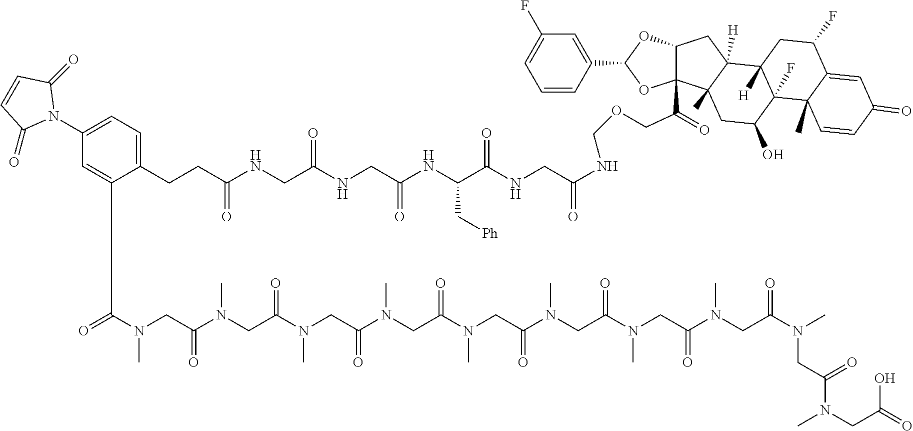 |
| I-54 | 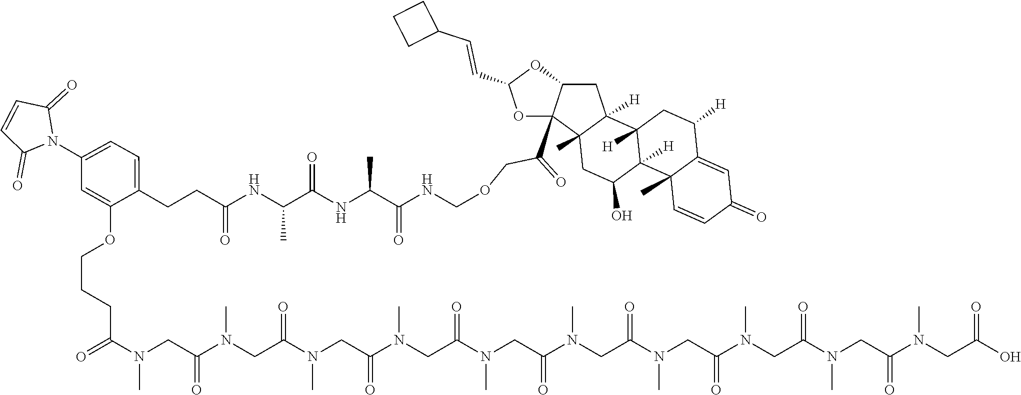 |
| I-55 | 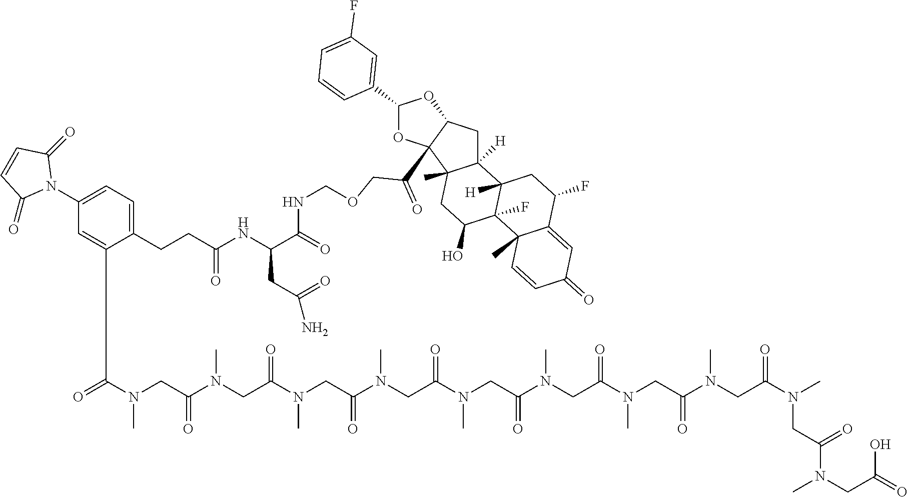 |
| I-56 | 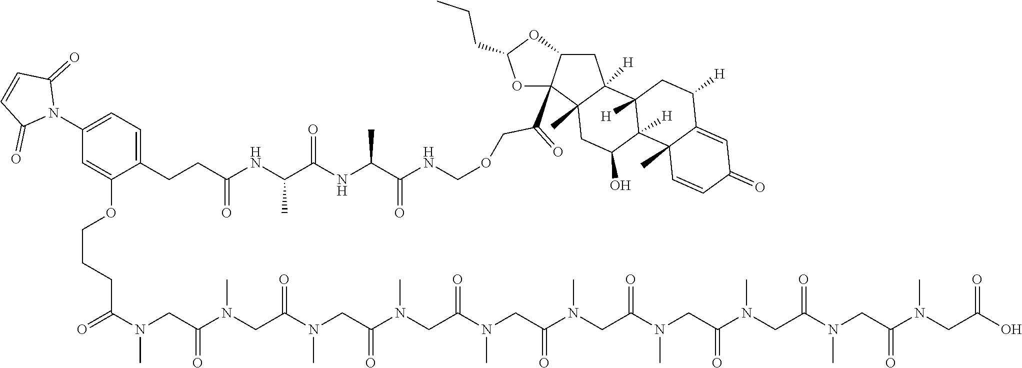 |
| I-57 | 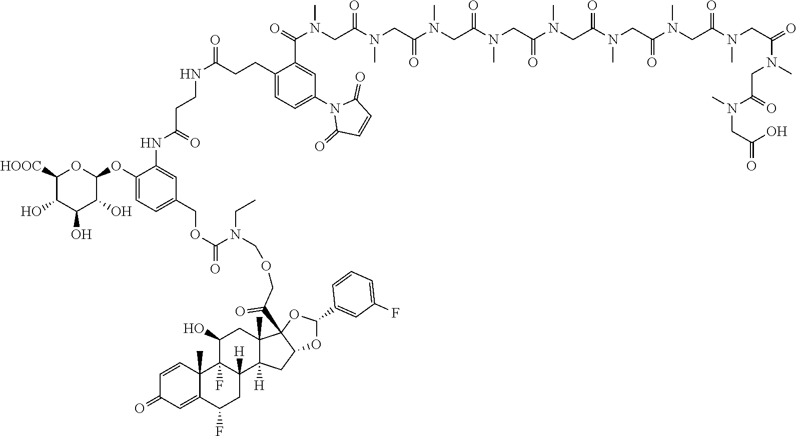 |
| I-58 | 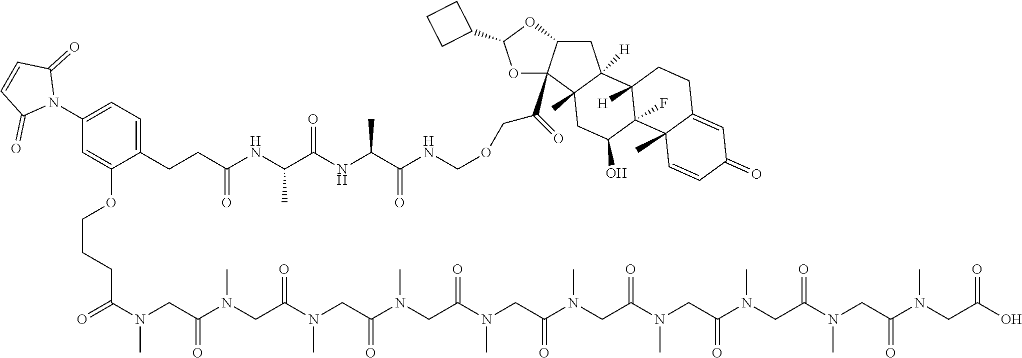 |
| I-59 | 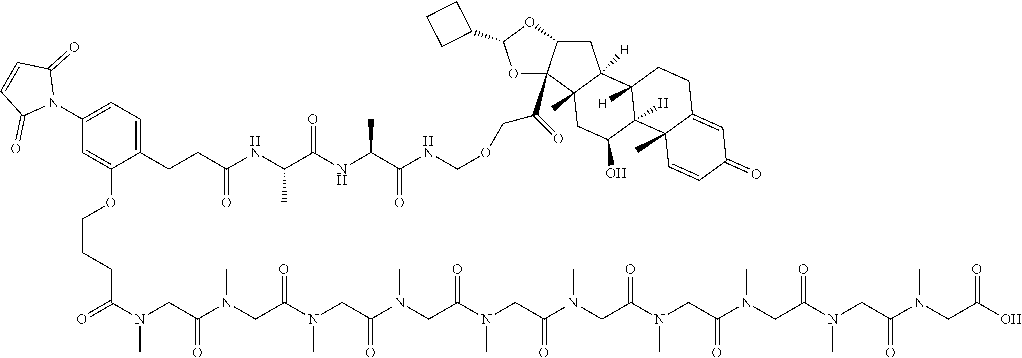 |
| I-60 | 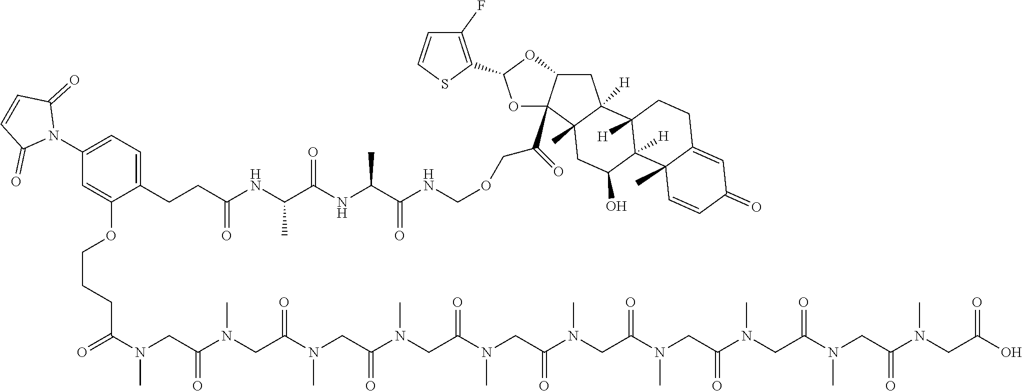 |
| I-61 | 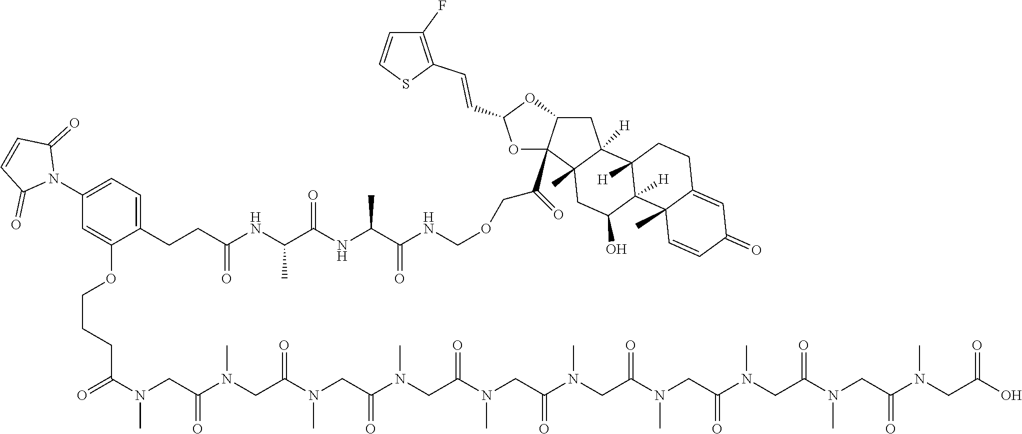 |
| I-62 | 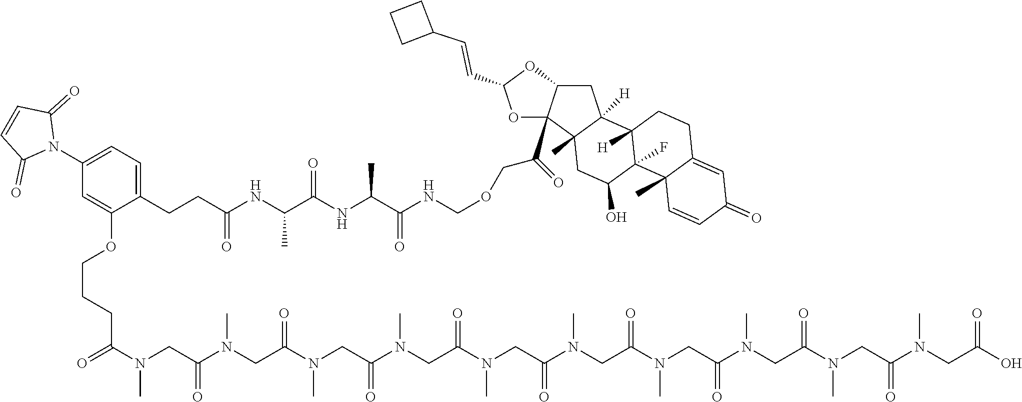 |
| I-63 | 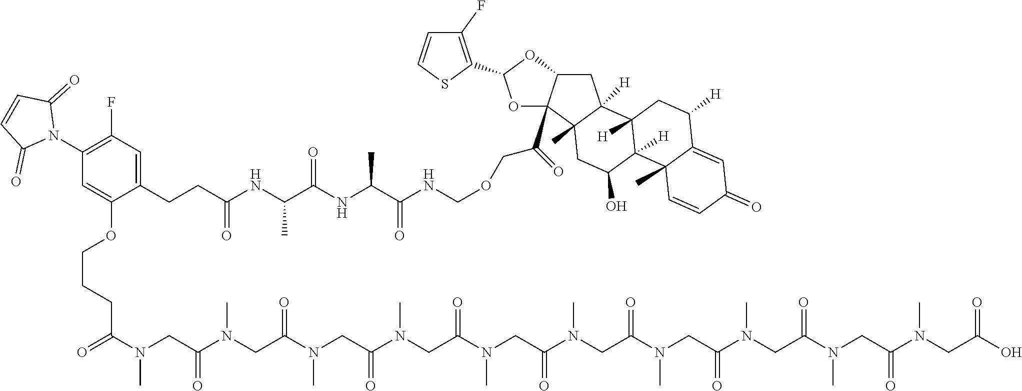 |
| I-64 | 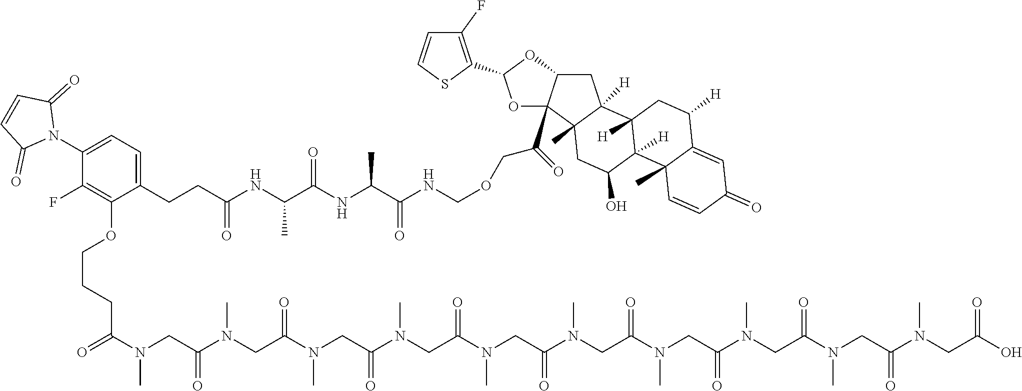 |
| I-65 | 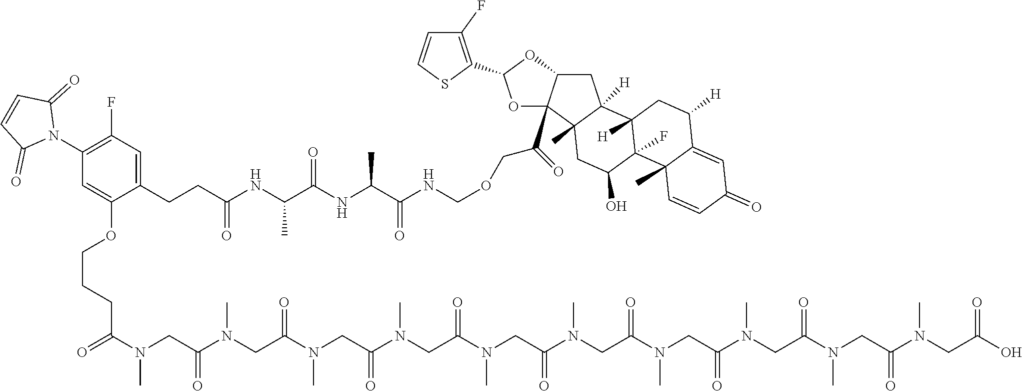 |
| I-66 | 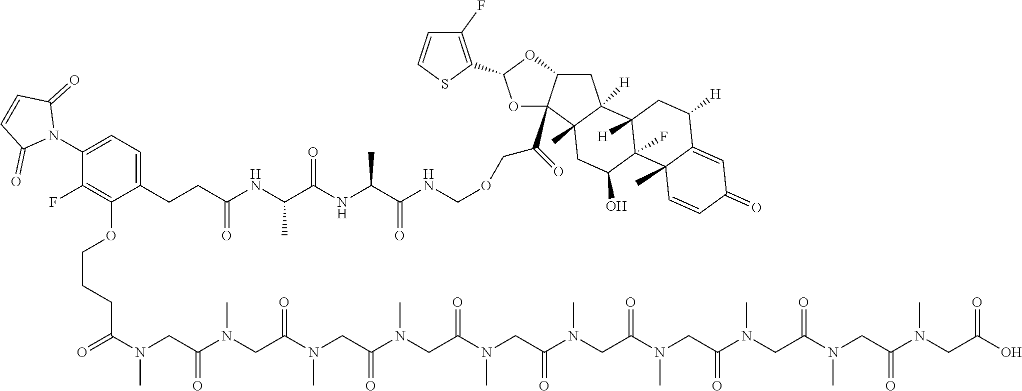 |
| I-67 | 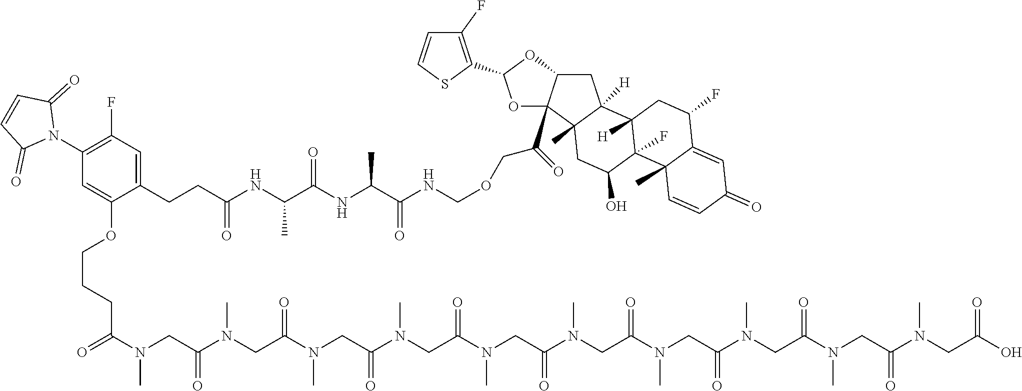 |
| I-68 | 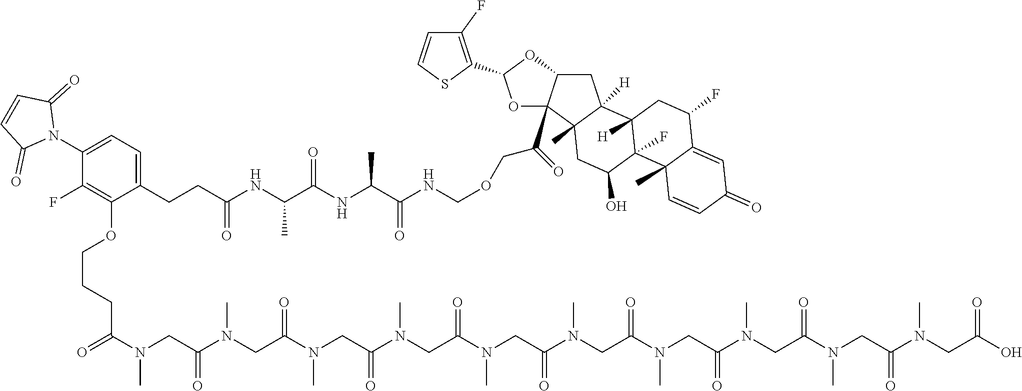 |
| I-69 | 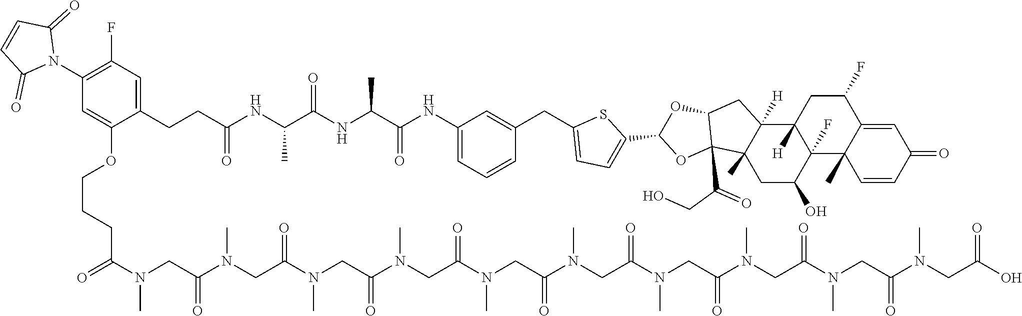 |
| I-70 | 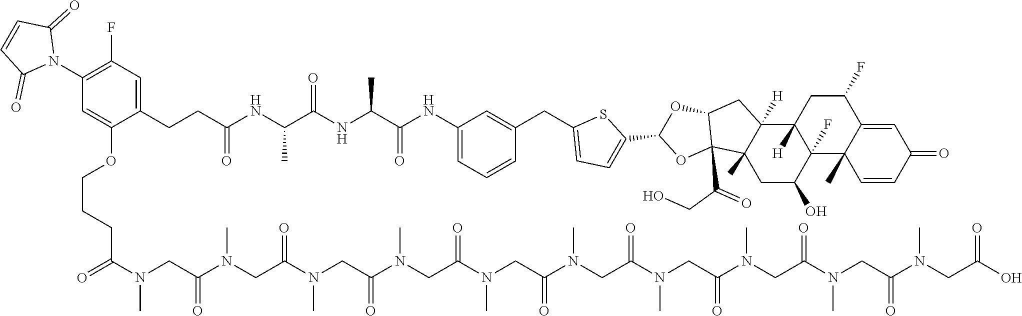 |
| I-71 | 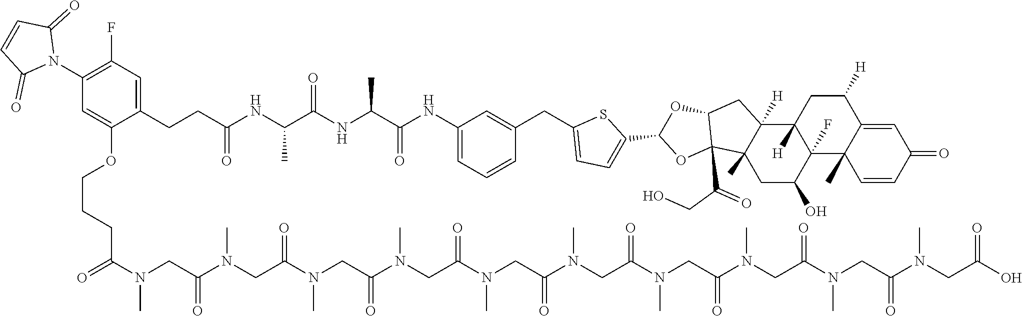 |
| I-72 | 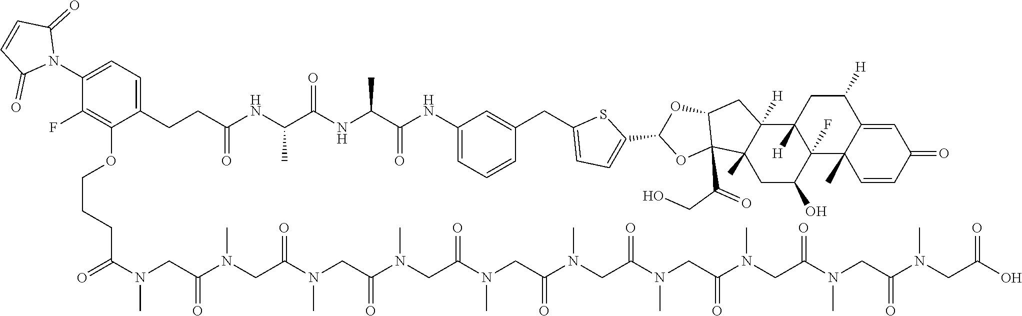 |
| I-73 | 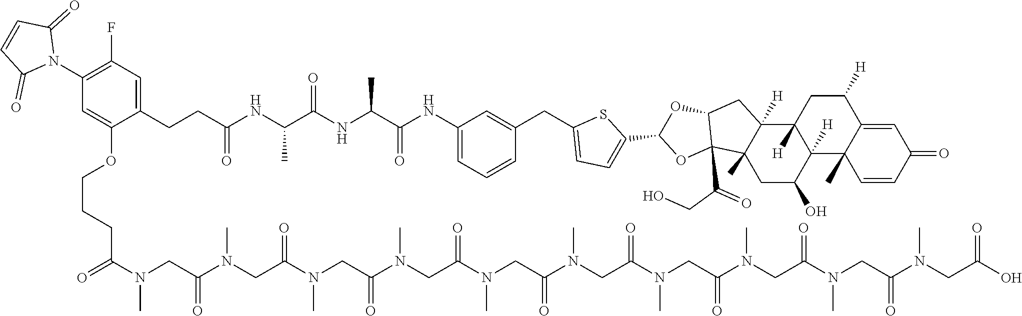 |
| I-74 | 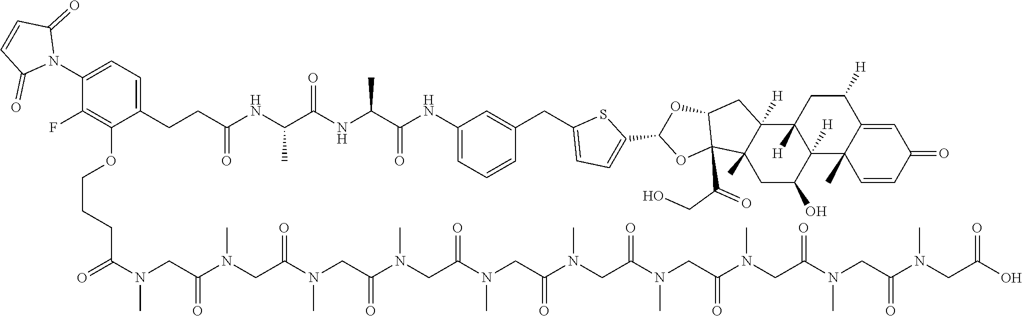 |
| I-75 | 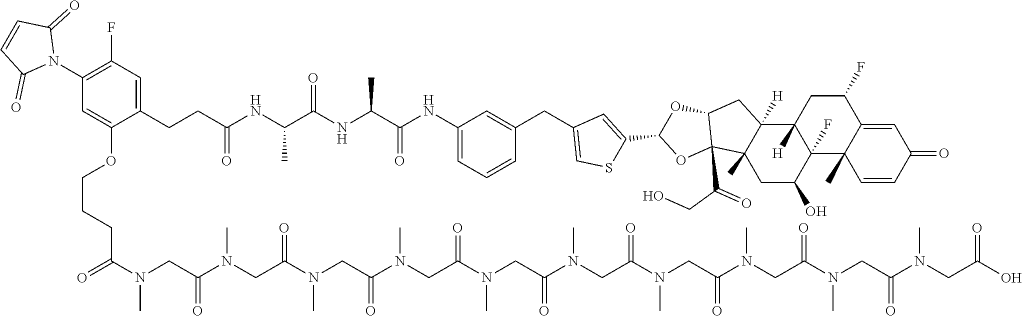 |
| I-76 | 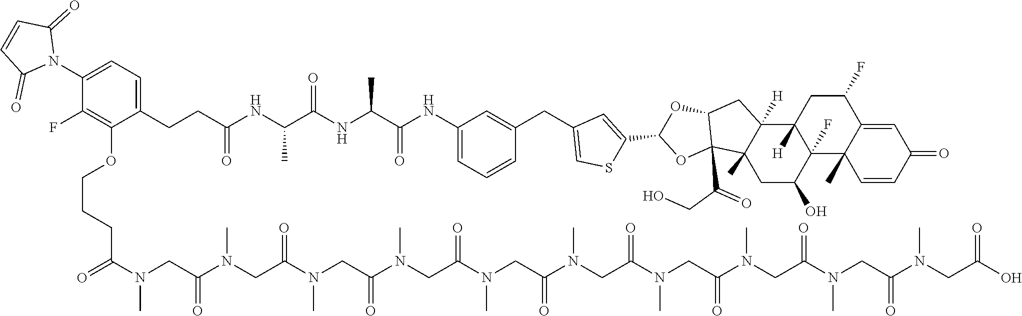 |
| I-77 | 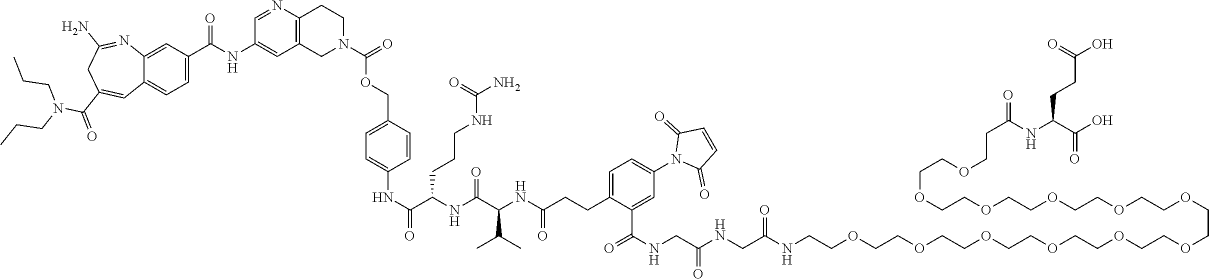 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCc5ccc(N6C(=O)C=CC6=O)cc5C(=O)NCC(=O)NCC(=O)NCCOCCOCCOCCOCCOCCOCCOCCOCCOCCOCCOCCOCCC(=O)N[C@@H](CCC(=O)O)C(=O)O)C(C)C)cc3)CC4)cc2N=C(N)C1] |

^{‡} Characterization data for Compounds I-35-55 and I-57-62 is as follows:

Compound I-35

MS m/z [M−H]⁻ (ESI): 1977.90; ¹H NMR (300 MHz, Methanol-d₄) δ 0.79 (d, J=7.1 Hz, 3H), 0.89 (s, 3H), 1.08 (t, J=7.1 Hz, 2H), 1.13-1.37 (m, 7H), 1.47 (s, 4H), 1.62-1.76 (m, 3H), 1.73-1.89 (m, 1H), 2.11 (d, J=12.7 Hz, 3H), 2.24 (t, J=7.2 Hz, 3H), 2.31-2.45 (m, 2H), 2.49-2.62 (m, 2H), 2.78-3.16 (m, 4H), 3.28 (d, J=5.4 Hz, 2H), 3.40-3.48 (m, 2H), 3.48-3.58 (m, 36H), 3.56-3.74 (m, 6H), 3.79 (d, J=11.7 Hz, 3H), 3.98 (s, 2H), 4.15-4.30 (m, 2H), 4.30-4.45 (m, 2H), 4.55-4.77 (m, 4H), 4.95 (d, J=4.3 Hz, 1H), 5.39 (s, 1H), 5.51 (s, 1H), 6.13-6.24 (m, 2H), 6.88 (s, 1H), 7.01 (t, J=8.9 Hz, 1H), 7.07-7.25 (m, 7H), 7.23-7.32 (m, 1H), 7.32-7.49 (m, 2H)

Compound I-36

MS m/z [M−H]⁻ (ESI): 1834.50; ¹H NMR (300 MHz, Methanol-d₄) δ: 0.75-1.12 (m, 3H), 1.20-1.43 (m, 1H), 1.51-1.68 (m, 4H), 1.70-1.84 (m, 2H), 1.85-2.06 (m, 2H), 2.05-2.42 (m, 6H), 2.45-2.61 (m, 4H), 2.69 (d, J=31.9 Hz, 1H), 3.08 (s, 2H), 3.41 (t, J=5.7 Hz, 2H), 3.53-3.59 (m, 2H), 3.59-3.66 (m, 43H), 3.74 (t, J=6.2 Hz, 2H), 3.90 (d, J=21.1 Hz, 4H), 4.11 (d, J=4.1 Hz, 2H), 4.23 (dd, J=7.9 Hz, 4.2 Hz, 1H), 4.32 (d, J=9.7 Hz, 1H), 4.92-5.16 (m, 4H), 5.34-5.54 (m, 1H), 5.60 (s, 1H), 5.90-6.03 (m, 1H), 6.26-6.63 (m, 3H), 6.82-7.46 (m, 6H), 7.56-7.66 (m, 1H), 7.73-7.88 (m, 1H)

Compound I-37

MS m/z [M−H]⁻ (ESI): 1801.60; ¹H NMR (300 MHz, Methanol-d₄) δ: 0.98 (s, 3H), 1.27-1.34 (m, 6H), 1.57 (s, 4H), 1.80-1.91 (m, 4H), 2.10-2.20 (m, 2H), 2.37-2.39 (m, 4H), 2.63-2.70 (m, 5H), 3.13-3.20 (m, 2H), 3.37-3.40 (m, 3H), 3.52-3.59 (m, 6H), 3.61-3.62 (m, 37H), 3.72-3.74 (m, 2H), 3.92 (s, 3H), 4.09 (s, 2H), 4.17-4.23 (m, 3H), 4.26-4.43 (m, 2H), 4.66-4.75 (m, 4H), 5.03-5.04 (m, 1H), 5.50-5.58 (m, 2H), 6.31-6.34 (m, 2H), 6.97 (s, 2H), 7.10-7.20 (m, 2H), 7.32-7.47 (m, 6H).

Compound I-38

MS m/z [M−H]⁻ (ESI): 977.30; ¹H NMR (300 MHz, Methanol-d₄) 0.99 (s, 3H), 1.56-1.60 (m, 4H), 1.76-1.78 (m, 2H), 1.91 (d, J=13.4 Hz, 1H), 2.31-2.34 (m, 3H), 2.49-2.53 (m, 2H), 2.84-2.93 (m, 3H), 3.41-3.44 (m, 2H), 3.80 (s, 3H), 4.02-4.04 (m, 2H), 4.30-4.33 (m, 1H), 4.86-4.99 (m, 1H), 5.02-5.05 (m, 2H), 5.50-5.59 (m, 2H), 6.30-6.34 (m, 3H), 6.81-6.93 (m, 3H), 7.01-7.21 (m, 3H), 7.33-7.38 (m, 3H).

Compound I-39

MS m/z [M−H]⁻ (ESI): 949.10; ¹H NMR (300 MHz, Methanol-d₄) δ: 0.99 (s, 3H), 1.20-1.30 (m, 1H), 1.57 (s, 4H), 1.70-1.78 (m, 2H), 1.90-2.00 (m, 1H), 2.15-2.35 (m, 3H), 2.60-2.70 (m, 1H), 3.60-3.66 (m, 2H), 3.97-3.99 (m, 3H), 4.13-4.15 (m, 2H), 4.30-4.35 (m, 1H), 5.01-5.05 (m, 2H), 5.50-5.59 (m, 2H), 6.30-6.34 (m, 3H), 6.52-6.60 (m, 1H), 7.16-7.34 (m, 6H), 7.61 (s, 1H), 7.89-7.92 (m, 1H).

Compound I-40

MS m/z [M−H]⁻ (ESI): 1121.35; ¹H NMR (300 MHz, Methanol-d₄) δ: 0.98 (s, 3H), 1.48-1.62 (m, 4H), 1.72-1.88 (m, 3H), 2.12-2.27 (m, 1H), 2.28-2.39 (m, 2H), 2.46-2.79 (m, 3H), 2.81-2.92 (m, 2H), 2.93-3.03 (m, 1H), 3.14-3.26 (m, 1H), 3.69-3.99 (m, 9H), 4.21-4.54 (m, 3H), 4.64-4.82 (m, 3H), 4.99-5.09 (m, 1H), 5.38-5.70 (m, 2H), 6.19-6.36 (m, 3H), 6.43-6.59 (m, 1H), 6.89-7.02 (m, 1H), 7.03-7.16 (m, 2H), 7.17-7.42 (m, 10H).

Compound I-41

MS m/z [M−H]− (ESI): 945.30; 1H NMR (400 MHz, Methanol-d4) δ: 0.99 (s, 3H), 1.27-1.29 (m, 3H), 1.35-1.36 (m, 3H), 1.56 (s, 4H), 1.78-1.81 (m, 3H), 2.15-2.25 (m, 1H), 2.30-2.34 (m, 2H), 2.50-2.54 (m, 2H), 2.60-2.70 (m, 1H), 2.91-2.99 (m, 2H), 3.81 (s, 3H), 4.21-4.28 (m, 3H), 4.39-4.44 (m, 1H), 4.68-4.77 (m, 3H), 5.04-5.05 (m, 1H), 5.50-5.58 (m, 2H), 6.30-6.33 (m, 2H), 6.79-6.81 (m, 1H), 6.89-6.94 (m, 3H), 7.11-7.18 (m, 1H), 7.20-7.29 (m, 2H), 7.31-7.39 (m, 3H).

Compound I-42

MS m/z [M/2-H]⁻ (ESI): 1050.05; ¹H NMR (300 MHz, Methanol-d₄) δ: 0.71-0.93 (m, 4H), 1.08 (t, J=7.1 Hz, 4H), 1.17-1.31 (m, 1H), 1.47 (s, 4H), 1.67 (s, 3H), 1.76-1.95 (m, 2H), 2.00-2.17 (m, 2H), 2.19-2.33 (m, 4H), 2.32-2.55 (m, 6H), 3.04 (t, J=7.3 Hz, 2H), 3.25-3.39 (m, 6H), 3.47-3.57 (m, 42H), 3.59-3.72 (m, 4H), 3.82 (s, 2H), 4.00 (s, 2H), 4.15-4.37 (m, 4H), 4.63 (s, 2H), 4.76-5.07 (m, 6H), 5.41 (q, J=23.0, 21.6 Hz, 3H), 6.24 (d, J=19.0 Hz, 3H), 6.80 (s, 2H), 6.88-7.09 (m, 3H), 7.11-7.39 (m, 7H), 8.09 (s, 1H)

Compound I-43

MS m/z [M−H]⁻ (ESI): 1105.40; ¹H NMR (300 MHz, Methanol-d₄) δ: 0.88-0.98 (m, 6H), 0.99-1.02 (m, 1H), 1.03-1.19 (m, 1H), 1.27 (s, 1H), 1.39-1.44 (m, 2H), 1.47 (s, 3H), 1.51-1.64 (m, 4H), 1.66-1.73 (m, 1H), 1.81-1.89 (m, 3H), 2.02-2.33 (m, 4H), 2.31-2.39 (m, 1H), 2.46-2.53 (m, 2H), 2.58-2.69 (m, 3H), 2.91-3.09 (m, 3H), 3.11-3.19 (m, 1H), 3.21-3.29 (m, 1H), 3.71-2.91 (m, 6H), 3.92-4.11 (m, 2H), 4.39-4.41 (m, 1H), 4.48-4.58 (m, 2H), 4.61-4.66 (m, 1H), 4.69-4.79 (m, 2H), 5.99 (s, 1H), 6.12-6.28 (m, 1H), 6.76-6.88 (m, 1H), 6.89-6.99 (m, 3H), 7.11-7.31 (m, 6H), 7.32-7.44 (m, 1H)

Compound I-44

[M−H]⁻ (ESI): 1193.40. ¹H NMR (300 MHz, Methanol-d4) δ 0.98 (s, 3H), 1.57 (s, 4H), 1.64-1.90 (m, 3H), 1.99-2.23 (m, 3H), 2.24-2.4 (m, 2H), 2.42-2.82 (m, 5H), 2.86-3.08 (m, 3H), 3.20 (dd, J=13.9 Hz, 5.8 Hz, 1H), 3.68-3.95 (m, 6H), 4.02 (t, J=6.1 Hz, 2H), 4.24-4.38 (m, 1H), 4.39-4.52 (m, 2H), 4.62 (d, J=28.1 Hz, 1H), 4.70-4.81 (m, 2H), 5.04 (d, J=4.4 Hz, 1H), 5.37-5.71 (m, 2H), 6.16-6.35 (m, 2H), 6.81 (dd, J=8.0 Hz, 1.9 Hz, 1H), 6.88-6.98 (m, 3H), 7.03-7.17 (m, 1H), 7.14-7.44 (m, 10H).

Compound I-45

MS m/z [M−H]⁻ (ESI): 1816.80; ¹H NMR (400 MHz, Methanol-d₄) δ: 0.79-0.88 (m, 6H), 0.89-0.92 (m, 1H), 0.94-1.04 (m, 1H), 1.13-1.24 (m, 5H), 1.26-1.34 (m, 3H), 1.37 (s, 3H), 1.42-1.51 (m, 4H), 1.58-1.62 (m, 1H), 1.69-1.71 (m, 2H), 1.91-2.19 (m, 5H), 2.21-2.29 (m, 1H), 2.46-2.61 (m, 4H), 2.79-2.88 (m, 15H), 2.89-2.99 (m, 13H), 3.11-3.18 (m, 1H), 3.66-3.81 (m, 6H), 3.91-4.01 (m, 4H), 4.02-4.18 (m, 7H), 4.21-4.24 (m, 3H), 4.26-4.32 (m, 5H), 4.39-4.48 (m, 2H), 4.49-4.58 (m, 3H), 4.61-4.68 (m, 3H), 4.71-4.76 (m, 2H), 5.89 (s, 1H), 5.99-6.22 (m, 1H), 6.61-6.78 (m, 1H), 6.79-6.98 (m, 3H), 7.01-7.18 (m, 2H), 7.19-7.24 (m, 4H), 7.26-7.38 (m, 1H)

Compound I-46

[M−H]⁻ (ESI): 1904.75; ¹H NMR (300 MHz, Methanol-d₄) δ 0.98 (s, 4H), 1.29 (s, 1H), 1.56 (s, 4H), 1.79 (d, J=13.8 Hz, 3H), 2.01-2.24 (m, 4H), 2.33 (s, 2H), 2.52-2.78 (m, 5H), 2.81-3.24 (m, 33H), 3.61-3.90 (m, 6H), 3.90-4.23 (m, 12H), 4.24-4.53 (m, 11H), 4.66-4.81 (m, 3H), 5.03 (d, J=4.4 Hz, 1H), 5.60 (s, 2H), 6.28 (d, J=11.7 Hz, 2H), 6.74-7.16 (m, 5H), 7.16-7.42 (m, 10H).

Compound I-47

MS m/z [M+H]⁺ (ESI): 1019.38

Compound I-48

MS m/z [M+H]⁺ (ESI): 1728.05; ¹H NMR (400 MHz, Methanol-d₄) δ: 0.86-0.90 (m, 1H), 1.02 (s, 3H), 1.22-1.40 (m, 6H), 1.56 (s, 3H), 1.56-1.70 (m, 1H), 1.75-1.9 (m, 3H), 2.05-2.40 (m, 3H), 2.25-2.45 (m, 2H), 2.50-2.61 (m, 2H), 2.62-2.75 (m, 2H), 2.90-3.15 (m, 33H), 3.95-4.55 (m, 24H), 4.61-4.82 (m, 3H), 5.05-5.11 (m, 1H), 5.40-5.70 (m, 2H), 6.25-6.40 (m, 2H), 6.70-6.82 (m, 1H), 6.85-7.00 (m, 3H), 7.05-7.28 (m, 3H), 7.31-7.5 (m, 3H).

Compound I-49

MS m/z [M+H]⁺ (ESI): 2008.9; ¹H NMR (400 MHz, DMSO-d₆) δ 9.97-9.80 (m, 1H), 8.70-8.62 (m, 1H), 8.27-8.16 (m, 2H), 7.92-7.85 (m, 2H), 7.64-7.16 (m, 12H), 7.14-7.08 (m, 2H), 6.29 (dd, J=1.6, 10.4 Hz, 1H), 6.12 (s, 1H), 5.72-5.47 (m, 2H), 5.05-4.89 (m, 3H), 4.86-4.57 (m, 3H), 4.38-4.24 (m, 2H), 4.19-4.17 (m, 2H), 4.12 (t, J=7.6 Hz, 1H), 3.92-3.86 (m, 2H), 3.74-3.69 (m, 2H), 3.60-3.54 (m, 3H), 3.49-3.44 (m, 44H), 3.38 (t, J=5.6 Hz, 3H), 3.31-3.27 (m, 1H), 3.22-3.19 (m, 2H), 3.01-2.91 (m, 2H), 2.38-2.26 (m, 4H), 2.20-2.09 (m, 1H), 1.99-1.87 (m, 3H), 1.78-1.64 (m, 4H), 1.47 (s, 3H), 1.27 (d, J=7.2 Hz, 3H), 1.06 (t, J=6.8 Hz, 3H), 0.87-0.74 (m, 9H).

Compound I-50

MS m/z [M−H]⁻ (ESI): 1670.0; ¹H NMR (400 MHz, DMSO-d₆) δ 8.75-8.57 (m, 1H), 8.39-7.95 (m, 1H), 7.50-7.19 (m, 6H), 7.18-6.97 (m, 2H), 6.28 (d, J=10.4 Hz, 1H), 6.12 (s, 1H), 5.75-5.50 (m, 2H), 4.93 (d, J=4.0 Hz, 1H), 4.74-4.50 (m, 3H), 4.43-3.86 (m, 23H), 3.05-2.59 (m, 32H), 2.60-2.54 (m, 1H), 2.43 (s, 2H), 2.35-2.27 (m, 1H), 2.23-2.10 (m, 1H), 2.04-1.91 (m, 1H), 1.80-1.61 (m, 3H), 1.58-1.38 (m, 4H), 1.29-1.09 (m, 6H), 0.85 (s, 3H)

Compound I-51

MS m/z [M+H]⁺ (ESI): 993.4; ¹H NMR (DMSO-d₆, 400 MHz) δ 7.73 (d, J=1.6 Hz, 1H), 7.46-7.38 (m, 3H), 7.31-7.19 (m, 4H), 7.12 (s, 2H), 6.27 (dd, J=1.6, 10.0 Hz, 1H), 6.12 (s, 1H), 5.74-5.52 (m, 2H), 5.05 (dd, J=9.6, 18.8 Hz, 1H), 4.94 (d, J=4.0 Hz, 1H), 4.74 (dd, J=8.4, 18.8 Hz, 1H), 4.19 (d, J=8.8 Hz, 1H), 3.91 (q, J=6.0 Hz, 2H), 3.25 (t, J=5.6 Hz, 2H), 3.13 (t, J=8.0 Hz, 2H), 2.45-2.38 (m, 2H), 2.31-2.26 (m, 1H), 2.20-2.12 (m, 1H), 2.02-1.92 (m, 1H), 1.82 (d, J=14.0 Hz, 1H), 1.77-1.66 (m, 2H), 1.47 (s, 4H), 0.88 (s, 3H)

Compound I-52

MS m/z [M−H]⁻ (ESI): 1701.5896; ¹H NMR (DMSO-d₆, 400 MHz) δ 7.46-7.38 (m, 2H), 7.31-7.14 (m, 6H), 7.08-7.01 (m, 2H), 6.26 (d, J=9.6 Hz, 1H), 6.13 (s, 1H), 5.70-5.48 (m, 2H), 5.07-4.92 (m, 2H), 4.73 (dd, J=8.8 Hz, 18.0 Hz, 1H), 4.32-3.91 (m, 25H), 3.00-2.74 (m, 34H), 2.43 (t, J=7.6 Hz, 2H), 2.25-2.15 (m, 1H), 2.04 (d, J=14.0 Hz, 1H), 1.89 (d, J=14.0 Hz, 1H), 1.72 (d, J=8.0 Hz, 2H), 1.50 (s, 4H), 0.92 (s, 3H)

Compound I-53

MS m/z [M−H]⁻ (ESI): 1846.1; ¹H NMR (400 MHz, DMSO-d₆) δ 8.58 (s, 1H), 8.39-8.28 (m, 1H), 8.21-8.03 (m, 2H), 8.20-8.03 (m, 2H), 7.33-7.07 (m, 11H), 6.25 (d, J=9.6 Hz, 1H), 6.11 (s, 1H), 5.75-5.53 (m, 2H), 4.94 (d, J=4.8 Hz, 1H), 4.72-4.56 (m, 3H), 4.50-3.85 (m, 22H), 3.78-3.65 (m, 6H), 3.11-2.68 (m, 30H), 2.54-2.52 (m, 1H), 2.27-2.25 (m, 1H), 2.23-2.10 (m, 2H), 1.99 (d, J=12.8 Hz, 1H), 1.82-1.63 (m, 3H), 1.48 (s, 4H), 0.86 (s, 3H).

Compound I-54

MS m/z [M−H]⁻ (ESI): 1678.1; ¹H NMR (400 MHz, DMSO-d₆) δ7.31 (d, J=10.0 Hz, 1H), 7.22-7.13 (m, 1H), 7.05 (s, 2H), 6.85 (d, J=10.4 Hz, 1H), 6.78-6.70 (m, 1H), 6.21-6.11 (m, 1H), 6.05-5.96 (m, 1H), 5.92 (s, 1H), 5.32-5.22 (m, 1H), 4.91 (d, J=6.8 Hz, 1H), 4.71 (d, J=3.6 Hz, 1H), 4.65-4.40 (m, 3H), 4.38-3.96 (m, 24H), 3.01-2.66 (m, 36H), 2.45-2.26 (m, 4H), 2.09-1.90 (m, 6H), 1.82-1.64 (m, 6H), 1.61-1.47 (m, 3H), 1.35 (s, 3H), 1.27-1.11 (m, 7H), 1.05-0.88 (m, 2H), 0.79 (s, 3H).

Compound I-55

MS m/z [M+Na]⁺ (ESI): 1665.8; ¹H NMR (400 MHz, DMSO-d₆) δ 8.72-8.59 (m, 1H), 7.45-7.35 (m, 1H), 7.34-7.10 (m, 7H), 7.02 (s, 2H), 6.31-6.23 (m, 1H), 6.13 (s, 1H), 5.69-5.60 (m, 1H), 5.57-5.52 (m, 1H), 4.89 (s, 1H), 4.63-4.50 (m, 4H), 4.32-4.15 (m, 12H), 2.98-2.67 (m, 35H), 2.44-2.28 (m, 5H), 2.18-2.05 (m, 1H), 1.73-1.63 (m, 3H), 1.46-143 (m, 4H), 0.82 (s, 3H)

Compound I-57

MS m/z [M+H]⁺ (ESI): 1969.9; ¹H NMR (400 MHz, DMSO-d₆) δ 8.16 (s, 1H), 7.52-6.93 (m, 11H), 6.29 (dd, J=1.6, 10.0 Hz, 1H), 6.12 (s, 1H), 5.75-5.47 (m, 2H), 5.09-4.90 (m, 3H), 4.89-4.59 (m, 4H), 4.44-3.77 (m, 22H), 3.39-3.25 (m, 7H), 3.03-2.63 (m, 31H), 2.62-2.53 (m, 4H), 2.44-2.26 (m, 4H), 2.23-2.12 (m, 1H), 2.02-1.97 (m, 1H), 1.77-1.65 (m, 3H), 1.52-1.45 (m, 4H), 1.15-1.02 (m, 3H), 0.86 (s, 3H)

Compound I-58

MS m/z [M+H]⁺ (ESI): 1672.0; ¹H NMR (400 MHz, DMSO-d₆) δ 8.76-8.59 (m, 1H), 7.33-7.24 (m, 1H), 7.22-7.15 (m, 1H), 7.11 (s, 2H), 6.88 (br d, J=8.8 Hz, 1H), 6.76 (br dd, J=1.6, 7.6 Hz, 1H), 6.22 (dd, J=1.6, 10.0 Hz, 1H), 6.01 (s, 1H), 4.74 (br s, 1H), 4.69-4.42 (m, 4H), 4.38-4.14 (m, 13H), 4.13-3.86 (m, 12H), 3.06-2.67 (m, 32H), 2.61 (dt, J=5.2, 13.2 Hz, 1H), 2.47-2.37 (m, 3H), 2.36-2.21 (m, 2H), 2.07-1.64 (m, 13H), 1.55 (br d, J=8.4 Hz, 2H), 1.46 (s, 3H), 1.36-1.25 (m, 1H), 1.25-1.19 (m, 3H), 1.19-1.12 (m, 3H), 0.81 (s, 3H).

Compound I-59

MS m/z [M+Na]⁺ (ESI): 1675.9; ¹H NMR (400 MHz, DMSO-d₆) δ 8.75-8.57 (m, 1H), 7.30 (br d, J=10.0 Hz, 1H), 7.24-7.14 (m, 1H), 7.08 (s, 2H), 6.87 (br d, J=9.2 Hz, 1H), 6.75 (br dd, J=1.6, 7.6 Hz, 1H), 6.20-6.10 (m, 1H), 5.91 (s, 1H), 4.72 (br d, J=3.6 Hz, 1H), 4.67-4.39 (m, 4H), 4.37-4.14 (m, 13H), 4.12-3.82 (m, 12H), 3.06-2.62 (m, 33H), 2.47-2.37 (m, 3H), 2.34-2.19 (m, 2H), 2.13-1.66 (m, 13H), 1.65-1.43 (m, 3H), 1.35 (s, 3H), 1.25-1.18 (m, 3H), 1.18-1.10 (m, 3H), 1.01-0.82 (m, 2H), 0.79 (s, 3H).

Compound I-60

MS m/z [M+Na]⁺ (ESI): 1722.0; ¹H NMR (400 MHz, DMSO-d₆) δ8.74-8.60 (m, 1H), 7.57 (s, 1H), 7.32 (d, J=9.6 Hz, 1H), 7.18 (dd, J=3.2, 10.4 Hz, 1H), 7.12 (s, 2H), 6.97-6.86 (m, 2H), 6.80-6.71 (m, 1H), 6.21-6.14 (m, 1H), 5.96 (d, J=18.8 Hz, 2H), 5.73 (s, 1H), 4.87 (d, J=1.6 Hz, 1H), 4.71-4.50 (m, 4H), 4.31 (s, 6H), 4.22 (d, J=14.4 Hz, 9H), 2.94-2.75 (m, 35H), 2.43-2.38 (m, 3H), 2.34-2.29 (m, 2H), 2.06 (s, 5H), 1.98-1.88 (m, 3H), 1.82-1.68 (m, 4H), 1.65 (s, 3H), 1.38 (s, 4H), 1.24-1.11 (m, 9H), 0.85 (s, 4H).

Compound I-61

MS m/z [M−H]⁻ (ESI): 1724.1; ¹H NMR (400 MHz, DMSO-d₆) δ7.51-7.43 (m, 1H), 7.30 (d, J=10.0 Hz, 1H), 7.21-7.12 (m, 1H), 7.06 (s, 2H), 6.95 (d, J=6.0 Hz, 1H), 6.90 (d, J=16.0 Hz, 1H), 6.84 (d, J=10.0 Hz, 1H), 6.76-6.65 (m, 1H), 6.14 (d, J=9.6 Hz, 1H), 5.91 (s, 1H), 5.87-5.76 (m, 1H), 5.12 (d, J=6.4 Hz, 1H), 4.84-4.72 (m, 1H), 4.64-4.48 (m, 3H), 4.31-4.24 (m, 5H), 4.24-4.13 (m, 9H), 4.11-3.92 (m, 11H), 2.97-2.71 (m, 34H), 2.44-2.36 (m, 2H), 2.34-2.23 (m, 2H), 2.11-1.86 (m, 5H), 1.72 (s, 2H), 1.64-1.52 (m, 3H), 1.35 (s, 3H), 1.22-1.13 (m, 6H), 1.04-0.91 (m, 2H), 0.80 (s, 3H).

Compound I-62

MS m/z [M−H]⁻ (ESI): 1696.1; ¹H NMR (400 MHz, DMSO-d₆) δ7.33-7.27 (m, 1H), 7.24-7.17 (m, 1H), 7.10 (s, 2H), 6.88 (d, J=9.6 Hz, 1H), 6.83-6.72 (m, 1H), 6.28-6.20 (m, 1H), 6.09-6.00 (m, 2H), 5.27-5.17 (m, 1H), 4.99-4.89 (m, 1H), 4.82-4.74 (m, 1H), 4.67-4.44 (m, 3H), 4.32 (s, 4H), 4.28-4.15 (m, 10H), 4.14-3.90 (m, 12H), 3.03-2.72 (m, 34H), 2.6-2.58 (m, 1H), 2.45-2.29 (m, 4H), 2.07-1.89 (m, 6H), 1.88-1.51 (m, 9H), 1.50-1.46 (m, 3H), 1.41-1.29 (m, 1H), 1.27-1.15 (m, 6H), 0.82 (s, 3H).

### Conjugates

Compounds of the present disclosure (e.g., compounds of Structure (I)) are useful partly because they may be attached to a targeting moiety or targeting fragment thereof (e.g., an antibody). Such an attachment may be made by reducing a disulfide bond of the protein with an appropriate reagent (e.g., TCEP) and coupling with a compound of Structure (I) under appropriate conditions.

Accordingly, an additional embodiment provides a conjugate having the following Structure (II):

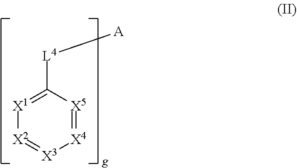

wherein.
A is a targeting moiety or a binding fragment thereof;
L⁴ has one of the following structures:

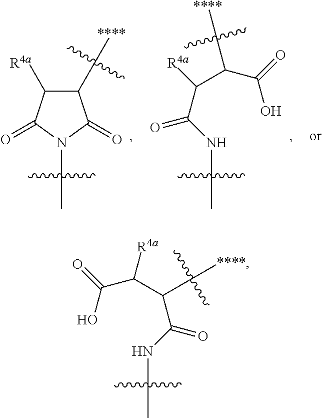

*** indicates an attachment point to A;
g is an integer from 1-20;
one of X¹, X², X³, X⁴ and X⁵ is C-L¹-R¹, another one of X¹, X², X³, X⁴ and X⁵ is C-L²-R², and the remaining three of X¹, X², X³, X⁴ and X⁵ are each independently N, C—R³, or C-L³-R^{3a};
R¹, R², and R^{3a} each independently comprise one or more moieties selected from an amino acid element, a charged element, a heteroalkylene element, a hydrophilic element, a trigger element, an immolative element, a polar cap, a payload, and combinations thereof; provided that at least one of R¹ and R² comprises a payload;
each occurrence of R³ is independently selected from the group consisting of hydrogen, deuterium, alkyl, haloalkyl, halo, alkoxy, haloalkoxy, amino, aminyl, amidyl, aldehyde, hydroxyl, cyano, nitro, thiol, carboxy, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, —P(O)₃H, alkyl-O—P(O)₃-alkyl, alkyl-P(O)₃-alkyl, —O-alkyl-S(O)₃-alkyl, —O-alkyl-O—P(O)₃-alkyl, —O-alkyl-P(O)₃-alkyl, —S(O)₃-alkyl, —OP(O)₃-alkyl, —P(O)₃-alkyl, sulfamide, sulfinimide, and a carbohydrate;
R^{4a} is hydrogen, deuterium, halo, or —S—R^{4c} wherein R^{4c} is substituted or unsubstituted C₆-C₁₀ aryl or substituted or unsubstituted 5-12 membered heteroaryl; and
L¹, L², and L³ are each independently a linker comprising an optionally substituted alkylene, an optionally substituted alkenylene, an optionally substituted alkynylene, an optionally substituted heteroalkylene, an optionally substituted heteroalkenylene, an optionally substituted heteroalkynylene, a heteroatomic linker, an optionally substituted cycloalkylene, an optionally substituted arylene, an optionally substituted heterocyclylene, an optionally substituted heteroarylene, or combinations thereof, as a stereoisomer, enantiomer or tautomer thereof or a mixture thereof; or a pharmaceutically acceptable salt, solvate or prodrug thereof.

An additional embodiment provides a conjugate having the following Structure (II):

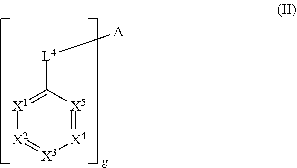

wherein:
A is a targeting moiety or a binding fragment thereof;
L⁴ has one of the following structures:

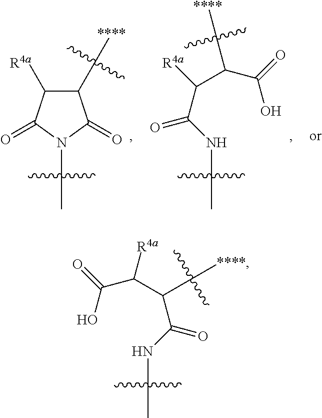

*** indicates an attachment point to A;
g is an integer from 1-20;
one of X¹, X², X³, X⁴ and X⁵ is C-L¹-R¹, another one of X¹, X², X³, X⁴ and X⁵ is C-L²-R², and the remaining three of X¹, X², X³, X⁴ and X⁵ are each independently N, C—R³, or C-L³-R^{3a};
R¹, R², and R^{3a} each independently comprise one or more moieties selected from an amino acid element, a charged element, a heteroalkylene element, a hydrophilic element, a trigger element, an immolative element, a polar cap, a payload, and combinations thereof; provided that at least one of R¹ and R² comprises a payload;
each occurrence of R³ is independently selected from the group consisting of hydrogen, deuterium, alkyl, haloalkyl, halo, alkoxy, haloalkoxy, amino, aminyl, amidyl, aldehyde, hydroxyl, cyano, nitro, thiol, carboxy, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, —P(O)₃H, alkyl-O—P(O)₃-alkyl, alkyl-P(O)₃-alkyl, —O-alkyl-S(O)₃-alkyl, —O-alkyl-O—P(O)₃-alkyl, —O-alkyl-P(O)₃-alkyl, —S(O)₃-alkyl, —OP(O)₃-alkyl, —P(O)₃-alkyl, sulfamide, sulfinimide, and a carbohydrate;
R^{4a} is hydrogen, deuterium, halo, or —S—R^{4c} wherein R^{4c} is substituted or unsubstituted C₆-C₁₀ aryl or substituted or unsubstituted 5-12 membered heteroaryl; and
L¹, L², and L³ are each independently a direct bond or a linker comprising an optionally substituted alkylene, an optionally substituted alkenylene, an optionally substituted alkynylene, an optionally substituted heteroalkylene, an optionally substituted heteroalkenylene, an optionally substituted heteroalkynylene, a heteroatomic linker, an optionally substituted cycloalkylene, an optionally substituted arylene, an optionally substituted heterocyclylene, an optionally substituted heteroarylene, or combinations thereof, as a stereoisomer, enantiomer or tautomer thereof or a mixture thereof; or a pharmaceutically acceptable salt, solvate or prodrug thereof.

In some embodiments, R¹ has the following structure:

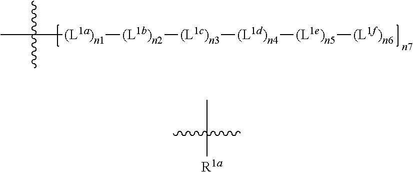

wherein:
L^{1a} is an amino acid element;
L^{1b} is a charged element;
L^{1c} is a heteroalkylene element;
L^{1d} is a hydrophilic element;
L^{1e} is a trigger element; and
L^{1f} is an immolative element;
   wherein one or more occurrence of L^{1a}, L^{1b}, L^{1c}, L^{1d}, L^{1e}, and L^{1f} optionally joins with one or more of another of L^{1a}, L^{1b}, L^{1c}, L^{1d}, L^{1e}, and L^{1f} to form one or more ring;
each occurrence of n1, n2, n3, n4, n5, and n6 is independently an integer from 0-3, provided that n1+n2+n3+n4+n5+n6=1 or more;
n7 is 1, 2, 3, 4, 5, or 6; and
R^{1a} is a payload that is covalently bound to one occurrence of L^{1a}, L^{1b}, L^{1c}, L^{1d}, L^{1e}, or L^{1f} and the payload is optionally substituted with a polar cap.

In certain embodiments, R¹ has the following structure:

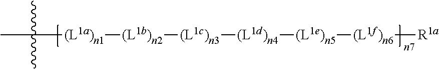

wherein:
L^{1a} is an amino acid element;
L^{1b} is a charged element;
L^{1c} is a heteroalkylene element;
L^{1d} is a hydrophilic element;
L^{1e} is a trigger element; and
L^{1f} is an immolative element;
   wherein one or more occurrence of L^{1a}, L^{1b}, L^{1c}, L^{1d}, L^{1e}, and L^{1f} optionally joins with one or more of another of L^{1a}, L^{1b}, L^{1c}, L^{1d}, L^{1e}, and L^{1f} to form one or more ring;
each occurrence of n1, n2, n3, n4, n5, and n6 is independently an integer from 0-3, provided that n1+n2+n3+n4+n5+n6=1 or more;
n7 is 1, 2, 3, 4, 5, or 6; and
R^{1a} is a payload optionally substituted with a polar cap.

In some embodiments, R² has the following structure:

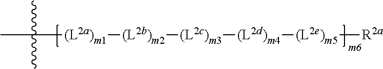

wherein:
L^{2a} is an amino acid element;
L^{2b} is a charged element;
L^{2c} is a heteroalkylene element;
L^{2d} is a hydrophilic element;
L^{2e} is a trigger element;
each occurrence of m1, m2, m3, m4, and m5 is independently an integer from 0-3, provided that m1+m2+m3+m4+m5=1 or more;
m6 is 1, 2, or 3; and
R^{2a} is hydrogen, alkyl, a payload, or a polar cap.

In certain embodiments, R^{3a} has the following structure:

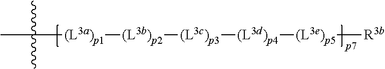

wherein:
L^{3a} is an amino acid element;
L^{3b} is a charged element;
L^{3c} is a heteroalkylene element;
L^{3d} is a hydrophilic element;
L^{3e} is a trigger element;
each occurrence of p1, p2, p3, p4, and p5 is independently an integer from 0-3, provided that p1+p2+p3+p4+p5=1 or more;
p6 is 1, 2, or 3; and
R^{3b} is hydrogen, alkyl, or a polar cap.

In some embodiments, n7 is 1 and each of n1 through n6 are 1. In certain embodiments, n7 is 1 and each of n1 through n4 are 0, n5 is 1, and n6 is 1. In some embodiments, n7 is 1 and n1 is 0, n2 is 0, n3 is 1, n4 is 0, n5 is 1, and n6 is 1.

In some embodiments, m6 is 1 and each of m1 through m5 are 1. In certain embodiments, m6 is 1, m1 is 1, m2 is 0, m3 is 0, m4 is 1, and m5 is 0.

In certain embodiments, p6 is 1 and each of p1 through p5 is 1. In some embodiments, p6 is 1, p1 is 1, p2 is 0, p3 is 0, p4 is 1, and p5 is 0.

In some embodiments, an amino acid element comprises one or more amino acids selected from the group consisting of glycine, alanine, serine, threonine, cysteine, valine, leucine, isoleucine, methionine, proline, phenylalanine, tyrosine, tryptophan, aspartic acid, glutamic acid, asparagine, glutamine, histidine, lysine, arginine, sarconsine, and beta-alanine. In more embodiments, an amino acid element comprises one or more amino acids selected from the group consisting of glycine, sarcosine, beta-alanine, and glutamic acid.

In some embodiments, an amino acid element comprises a dipeptide, a tripeptide, a tetrapeptide, or a pentapeptide. In some embodiments, an amino acid element has one of the following structures:

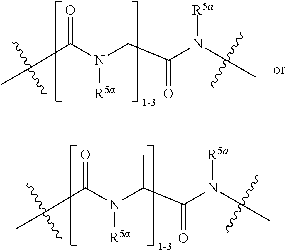

wherein:
each occurrence of R^{5a} is independently hydrogen, alkyl, hydroxyalkyl, or alkoxyalkyl.

In some embodiments, a charged element comprises moieties with a negative charge at pH 7.4 (i.e., a range from 6.3 to 8.5). In some embodiments, a charged element comprises moieties with a positive charge at pH 7.4 (i.e., a range from 6.3 to 8.5).

In some embodiments, a charged element comprises one or more charged amino acid, one or more carboxylic acid, one or more sulfonic acid, one or more sulfonamide, one or more sulfate, one or more phosphate, one or more quaternary amine, one or more sulfamide, one or more sulfinimide, or combinations thereof.

In some embodiments, a charged amino acid is aspartic acid, glutamic acid, histidine, lysine, or arginine.

In some embodiments, a heteroalkylene element comprises polyethylene glycol or polypropylene glycol. In some embodiments, a heteroalkylene element comprises one of the following structures:

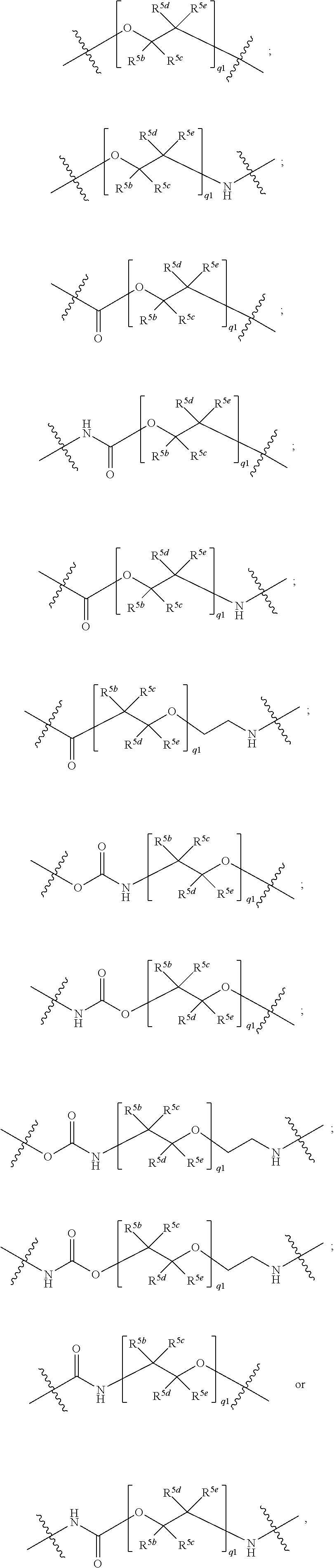

wherein:
each occurrence of R^{5b}, R^{5c} R^{5d}, and R^{5e} is independently selected from the group consisting of hydrogen, deuterium, alkyl, haloalkyl, halo, alkoxy, haloalkoxy, amino, hydroxyl, cyano, nitro, thiol, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, and —P(O)₃H,
each occurrence of q1 is, independently an integer from 1-24.

In some embodiments, q1 is 8, 10, 12, or 14.

In some embodiments, R¹, R², or R^{3a} comprises a non-cleavable linker.

In more embodiments, R¹, R², or R^{3a} comprises one of the following structures:

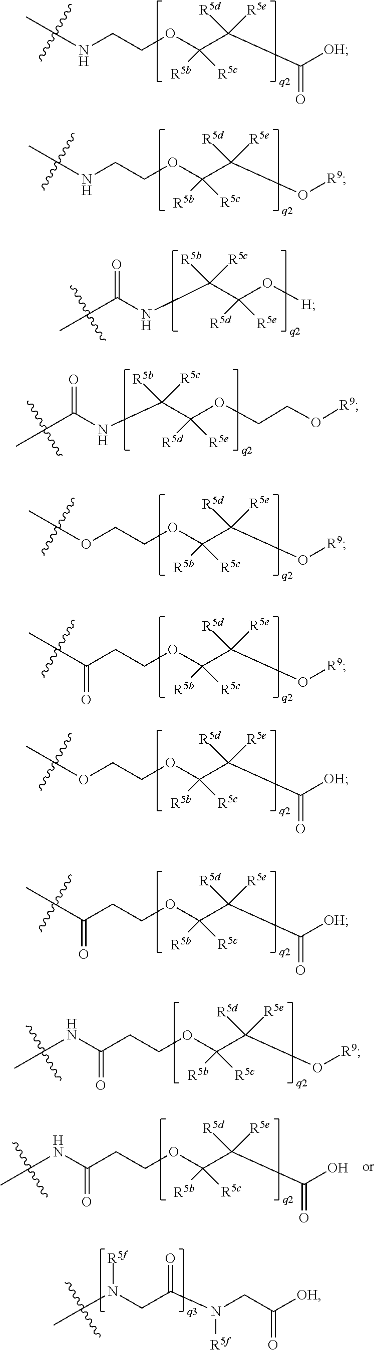

wherein:
each occurrence of R^{5b}, R^{5c} R^{5d}, and R^{5e} is independently selected from the group consisting of hydrogen, deuterium, alkyl, haloalkyl, halo, alkoxy, haloalkoxy, amino, hydroxyl, cyano, nitro, thiol, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, —P(O)₃H, alkyl-O—P(O)₃-alkyl, alkyl-P(O)₃-alkyl, —O-alkyl-S(O)₃-alkyl, —O-alkyl-O—P(O)₃-alkyl, —O-alkyl-P(O)₃-alkyl, —S(O)₃-alkyl, —OP(O)₃-alkyl, —P(O)₃-alkyl, sulfamide, sulfinimide;
each occurrence of R^{5f} is independently hydrogen, alkyl, hydroxyalkyl, or alkoxyalkyl;
each occurrence of R⁹ is independently hydrogen or alkyl;
each occurrence of q2 is independently an integer from 1-25; and
each occurrence of q3 is independently an integer from 5-15.

In some embodiments, q2 is 8, 10, 12, or 14. In some embodiments, q3 is 6, 8, 10, 12, or 14.

In some embodiments, a hydrophilic element comprises polyethylene glycol, polysarcosine, cyclodextrin, c-glycosides, or combinations thereof. In some embodiments, a hydrophilic element comprises one of the following structures:

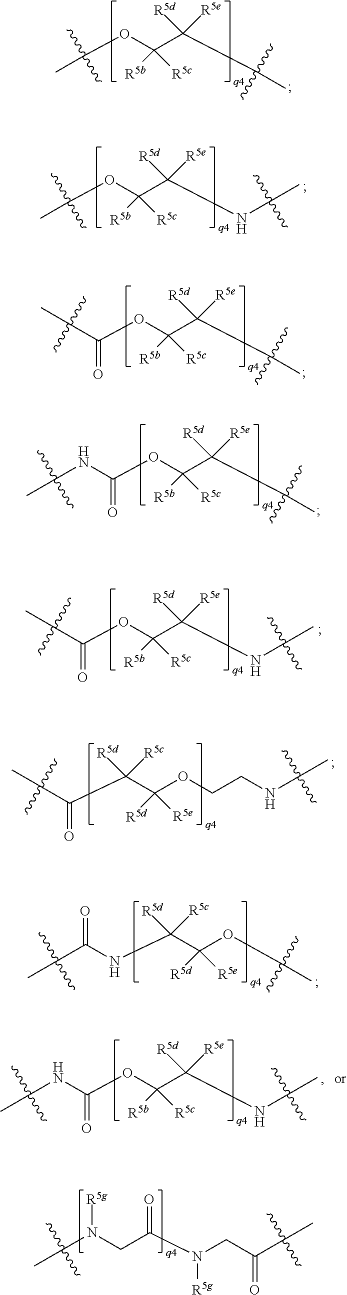

wherein:
each occurrence of R^{5b}, R^{5c} R^{5d}, and R^{5e} is independently selected from the group consisting of hydrogen, deuterium, alkyl, haloalkyl, halo, alkoxy, haloalkoxy, amino, hydroxyl, cyano, nitro, thiol, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, and —P(O)₃H,
each occurrence of R^{5g} is independently hydrogen, alkyl, hydroxyalkyl, or alkoxyalkyl; and
each occurrence of q4 is, independently an integer from 1-24.

In some embodiments, R^{5g} is —CH₃ at each occurrence.

In some embodiments, a trigger element comprises a dipeptide, a tripeptide, a tetrapeptide, a pentapeptide, a glucuronide, a disulfide, a phosphate, a diphosphate, a triphosphate, a hydrazone, or combinations thereof. In some embodiments, a trigger element comprises beta-glucuronic acid. In certain embodiments, a trigger element comprises a dipeptide, a tripeptide, a tetrapeptide, or a pentapeptide. In certain embodiments, a trigger element comprises two or more amino acids selected from the group consisting of valine, citrulline, alanine, glycine, phenylalanine, lysine, or combinations thereof. In some embodiments, a trigger element comprises a sequence of amino acids selected from the group consisting of valine-citrulline, valine-alanine, glycine-glycine-phenylalanine-glycine, and combinations thereof. In certain embodiments, a trigger element comprises one of the following structures, including combinations thereof:

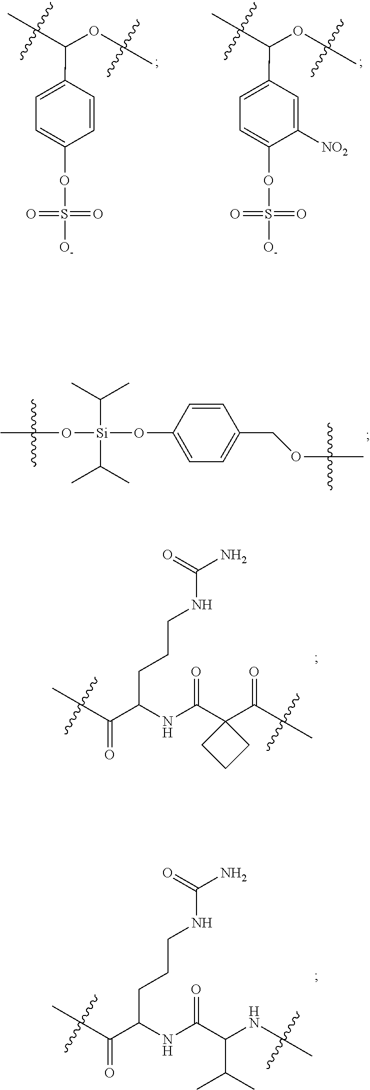

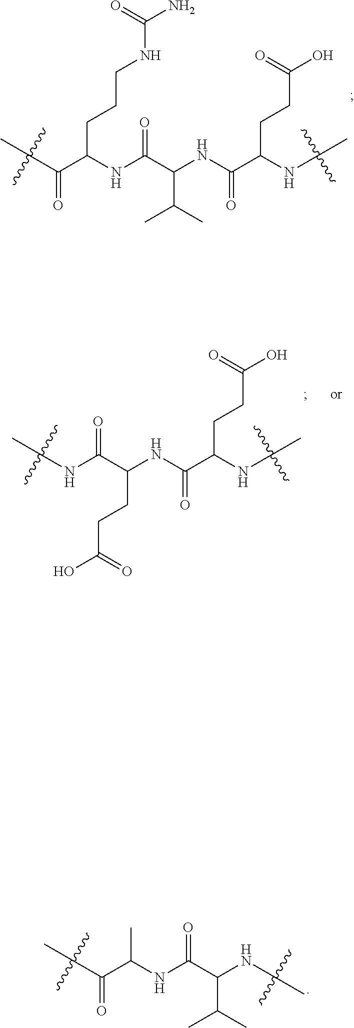 [SMILES: *NC(CCC(=O)O)C(=O)NC(C(=O)NC(CCCNC(N)=O)C(*)=O)C(C)C ; *NC(=O)C(CCC(=O)O)NC(=O)C(CCC(=O)O)N* ; *NC(C(=O)NC(C)C(*)=O)C(C)C]

In some embodiments, an immolative element comprises para-aminobenzyloxycarbonyl, an aminal, a hydrazine, a disulfide, an amide, an ester, a hydrazine, a phosphotriester, a diester, a β-glucuronide, a double bond, a triple bond, an ether bond, a ketone, a diol, a cyano, a nitro, a quaternary amine, or combinations thereof. In certain embodiments, an immolative element comprises a paramethoxybenzyl, a dialkyldialkoxysilane, a diaryldialkoxysilane, an orthoester, an acetal, an optionally substituted β-thiopropionate, a ketal, a phosphoramidate, a hydrazone, a vinyl ether, an imine, an aconityl, a trityl, a polyketal, a bis-arylhydrazone, a diazobenzene, a vivinal diol, a pyrophosphate diester, or combinations thereof.

In some embodiments, an immolative element comprises one of the following structures:

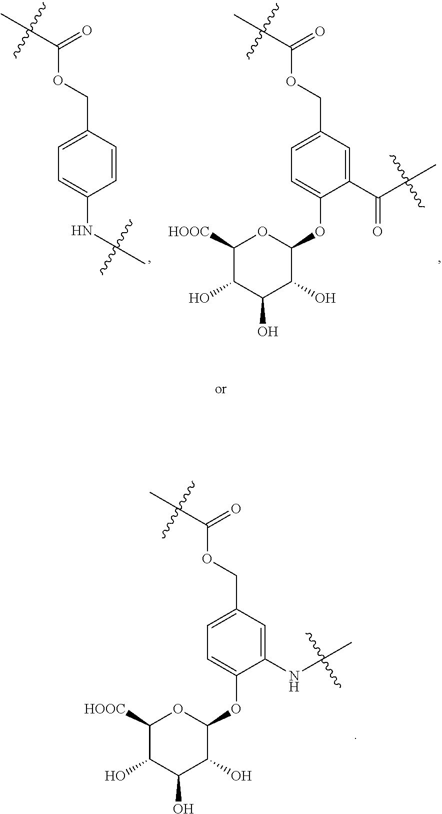 [SMILES: *Nc1ccc(COC(*)=O)cc1 ; *C(=O)OCc1ccc(O[C@@H]2O[C@H](C(=O)O)[C@@H](O)[C@H](O)[C@H]2O)c(C(*)=O)c1 ; *Nc1cc(COC(*)=O)ccc1O[C@@H]1O[C@H](C(=O)O)[C@@H](O)[C@H](O)[C@H]1O]

In certain embodiments, an immolative element comprises the following structure:

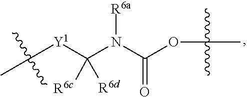

wherein:
R^{6a}, R^{6b}, R^{6c}, and R^{6d} are independently hydrogen, an optionally substituted alkyl, an optionally substituted aryl, or optionally substituted heteroaryl, or
R^{6a} and R^{6c} together with the nitrogen and carbon atoms to which they are attached form azetidinyl, pyrrolodinyl, piperidinyl, or homopiperidinyl and R^{6d} is hydrogen; and
Y¹ is —O—, —S—, or —NR^{6b}—.

In some embodiments, an immolative element comprises the following structure:

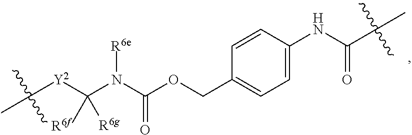

wherein:
R^{6e}, R^{6f}, R^{6g}, and R^{6h} are independently hydrogen, an optionally substituted alkyl, an optionally substituted aryl, or optionally substituted heteroaryl, or
R^{6a} and R^{6c} together with the nitrogen and carbon atoms to which they are attached form azetidinyl, pyrrolodinyl, piperidinyl, or homopiperidinyl and R^{6d} is hydrogen; and
Y² is —O—, —S—, or —NR^{6f}—.

In some embodiments, an immolative element comprises the following structure:

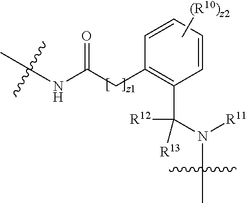

wherein:
each occurrence of R¹⁰ is independently alkyl, alkoxy, or halo;
R¹¹ is hydrogen, alkyl, or —(CH₂CH₂O)_{z3}—CH₃;
R¹² is hydrogen or alkyl;
R¹³ is hydrogen or alkyl;
z1 is 0 or 1;
z2 is 0, 1, 2, 3, or 4; and
z3 is 0, 1, 2, 3, 4, 5, 6, 7, 8, 9, or 10.

In some embodiments, an immolative element comprises one of the following structures:

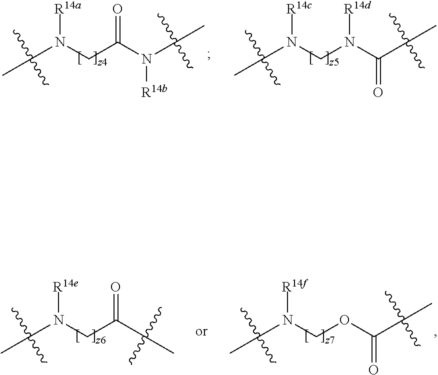

wherein:
R^{14a}, R^{14b}, R^{14c}, R^{14d}, R^{14e}, and R^{14f} are each independently hydrogen, alkyl, hydroxyalkyl, or alkoxyalkyl;
z4, z5, z6, and z7 are each independently 1, 2, 3, 4, 5, or 6.

In certain embodiments, an immolative element comprises one of the following structures:

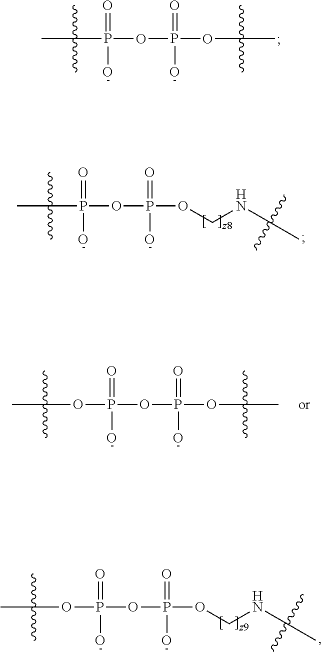

wherein:
z8 and z9 are each independently 1, 2, 3, 4, 5, or 6; or

In some embodiments, an immolative element comprises one of the following structures:

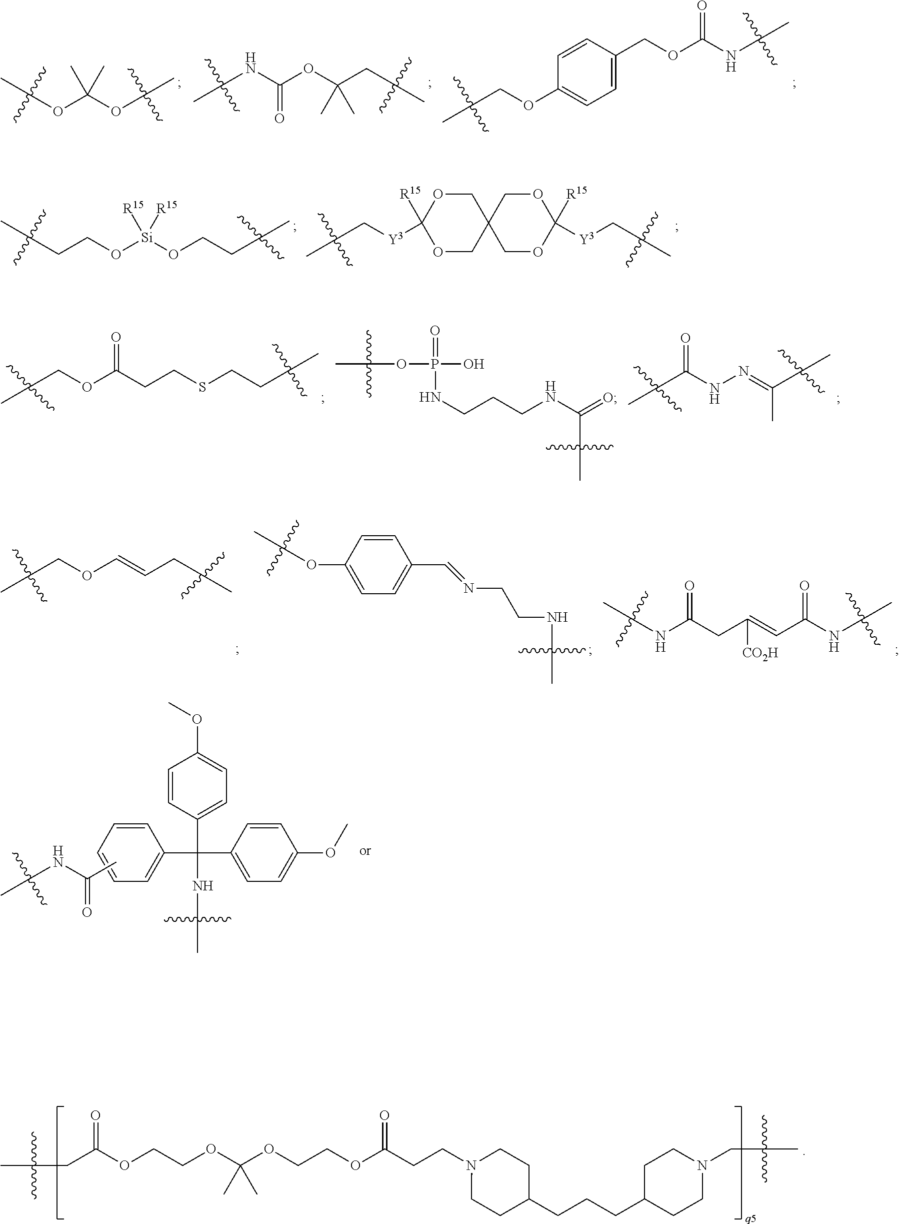

### Wherein:

each occurrence of R¹⁵ is independently H, methyl, ethyl, isopropyl, tert-butyl, or phenyl;
Y³ is O or CH₂; and
q5 is an integer ranging from 1-5.

In some embodiments, R^{4a} is hydrogen and ---- is a single bond. In some embodiments, R^{4a} is hydrogen and ---- is absent.

In some embodiments, a trigger element and an immolative element together comprise one of the following structures:

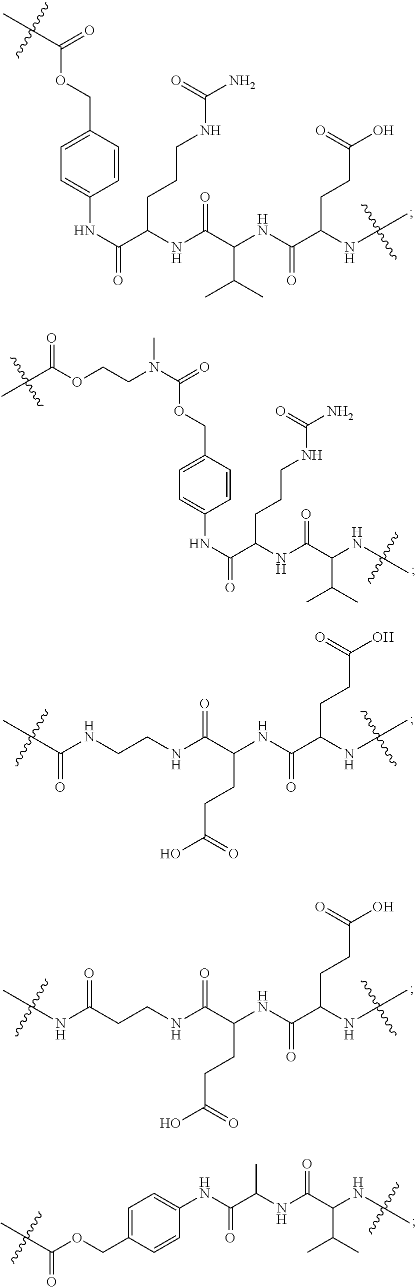

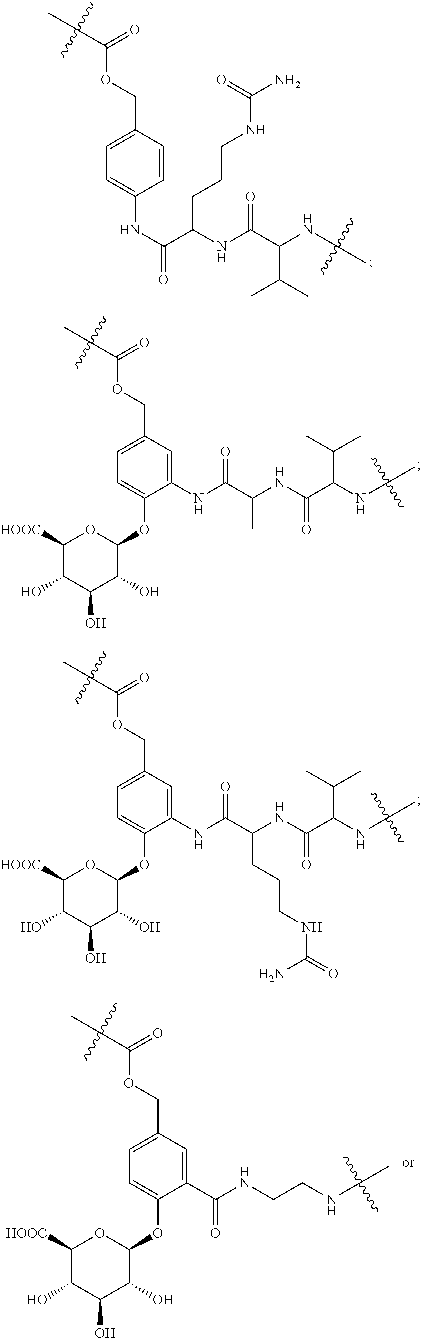 [SMILES: *NC(C(=O)NC(CCCNC(N)=O)C(=O)Nc1ccc(COC(*)=O)cc1)C(C)C ; *NC(C(=O)NC(C)C(=O)Nc1cc(COC(*)=O)ccc1O[C@@H]1O[C@H](C(=O)O)[C@@H](O)[C@H](O)[C@H]1O)C(C)C ; *NC(C(=O)NC(CCCNC(N)=O)C(=O)Nc1cc(COC(*)=O)ccc1O[C@@H]1O[C@H](C(=O)O)[C@@H](O)[C@H](O)[C@H]1O)C(C)C ; *NCCNC(=O)c1cc(COC(*)=O)ccc1O[C@@H]1O[C@H](C(=O)O)[C@@H](O)[C@H](O)[C@H]1O]

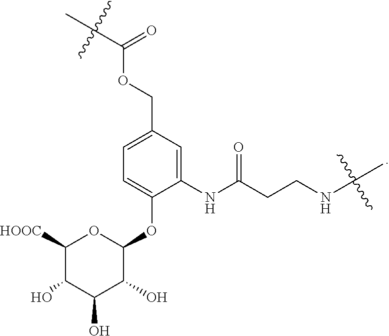 [SMILES: *NCCC(=O)Nc1cc(COC(*)=O)ccc1O[C@@H]1O[C@H](C(=O)O)[C@@H](O)[C@H](O)[C@H]1O]

In some embodiments, a polar cap comprises one or more charged amino acid, one or more polyol, or combinations thereof. In certain embodiments, a polar cap comprises a diol, a triol, a tetraol, or combinations thereof. In some embodiments, a polar cap comprises glycerol, trimethylolpropane, pentaerythritol, maltitol, sorbitol, xylitol, erythritol, isomalt, or combinations thereof. In certain embodiments, a polar cap comprises one or more natural amino acids. In some embodiments, a polar cap comprises one or more non-natural amino acids. In certain embodiments, a polar cap comprises one or more non-natural amino acids and one or more natural amino acids. In some embodiments, a polar cap comprises serine, threonine, cysteine, proline, asparagine, glutamine, lysine, arginine, histidine, aspartate, glutamate, 4-hydroxyproline, 5-hydroxylysine, homoserine, homocysteine, ornithine, beta-alanine, statine, or gamma aminobutyric acid. In certain embodiments, a polar cap comprises aspartic acid, serine, glutamic acid, serine-beta-glucose, or combinations thereof.

In some embodiments, a polar cap has one of the following structures, including combinations thereof:

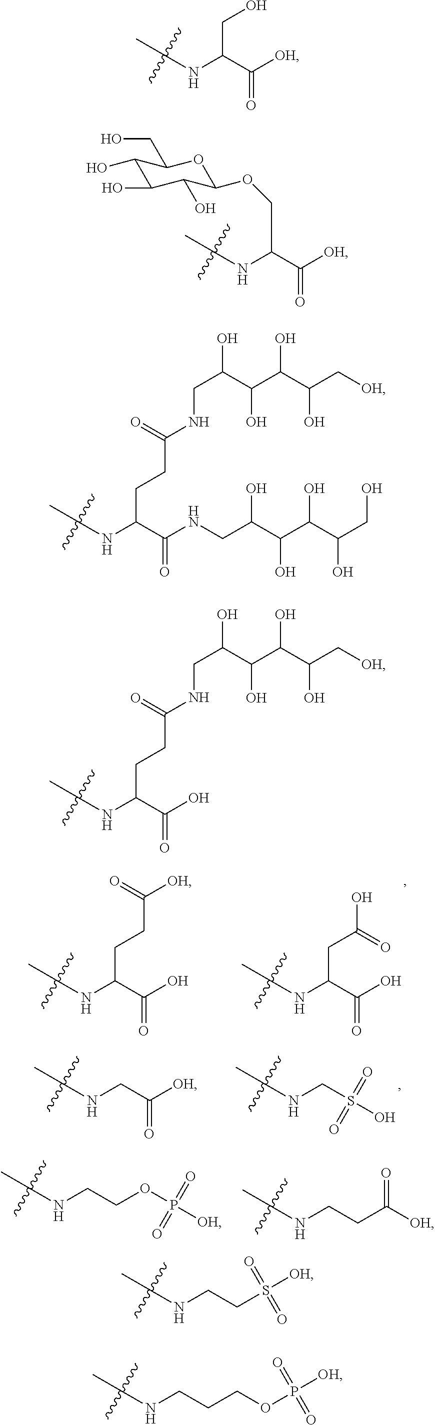

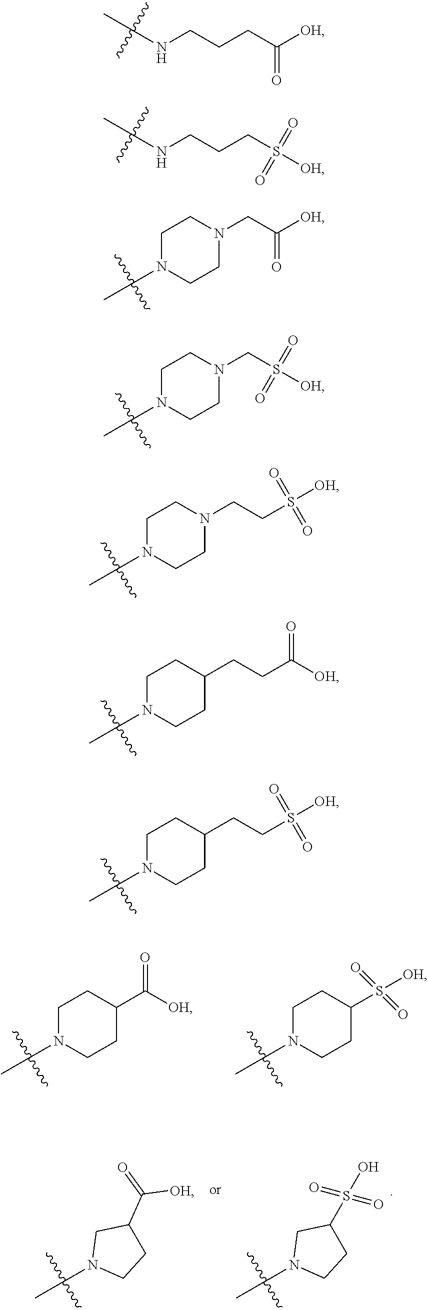

In some embodiments, a payload is a chemotherapeutic, a cytotoxic agent, or a myeloid cell agonist.

In some embodiments, a payload is a chemotherapeutic selected from the group consisting of camptothecin, paclitaxel, doxorubicin, vinblastine, dacarbazine, irinotecan, topotecan, silatecan, cositecan, Exatecan, Lurtotecan, SN-38, Dxd, gimatecan, Belotecan, and Rubitecan.

In still other embodiments, a payload is a cytotoxic agent selected from the group consisting of calicheamicin, anthramycin, abbeymycin, chicamycin, DC-81, mazethramycin, neothramycin A, neothramycin B, porothramycin prothracarcin, sibanomicin, sibiromycin, tomamycin, auristatin F, monomethyl auristatin F, auristatin E, monomethyl auristatin E, dolastatin, monomethyl dolastatin, mertansine, and emtansine.

In some embodiments, a payload is a myeloid cell agonist selected from a STING agonist, a ligand of TLR2, a ligand of TLR3, a ligand of TLR4, a ligand of TLR5, a ligand of TLR6, a ligand of TLR7, a ligand of TLR8, a ligand of TLR9, a ligand of TLR10, a ligand of nucleotide-oligomerization domain (NOD), a ligand of an RIG-I-Like Receptors (RLR), a ligand of a C-type lectin receptor (CLR), a ligand of a Cytosolic DNA Sensor (CDS) and a ligand of an inflammasome inducer, preferably wherein the myeloid cell agonist is selected from the group consisting of selgantolimod, motolimod, resiquimod, 3M-051, 3M-052, MCT-465, IMO-4200, VTX-763, VTX-1463, RG7854, ADU-S100, MK-1454, MK-2118, BMS-986301, GSK3745417, SB-11285, and IMSA-101.

In some embodiments, L¹, L², or L³ comprise a linker selected from the group alkylene, alkylene-L^{a}-, alkenylene, alkenylene-L^{a}-, alkynylene, alkynylene-L^{a}-, -L^{a}-, -L^{a}-alkylene-L^{a}-, -L^{a}-alkenylene-L^{a}-, -L^{a}-alkynylene-L^{a}-, and combinations thereof, wherein each alkylene, alkenylene, and alkynylene is optionally substituted;
each occurrence of L^{a} is independently selected from —O—, —S—, —N(R⁷)—, —C(O)—, —C(S)—, —C(O)O—, —OC(O)—, —OC(O)O—, —C(O)N(R⁷)—, —N(R⁷)C(O)—, —C(O)N(R⁷)C(O)—, —C(O)N(R⁷)C(O)N(R⁷), —N(R⁷)C(O)N(R⁷)—, —N(R⁷)C(O)O—, —OC(O)N(R⁷)—, —C(NR⁷)—, —N(R⁷)C(NR⁷)—, —C(NR⁷)N(R⁷)—, —N(R⁷)C(NR⁷)N(R⁷)—, —S(O)₂—, —OS(O)—, —S(O)O—, —S(O), —OS(O)₂—, —S(O)₂O, —N(R⁷)S(O)₂—, —S(O)₂N(R⁷)—, —N(R⁷)S(O)—, —S(O)N(R⁷)—, —N(R⁷)S(O)₂N(R⁷)—, and —N(R⁷)S(O)N(R⁷)—;
R⁷ is independently selected at each occurrence from hydrogen, —NH₂, —C(O)OCH₂C₆H₅; and C₁₋₁₀ alkyl, C₂₋₁₀ alkenyl, C₂₋₁₀ alkynyl, C₃₋₁₂ cycloalkyl, and 3- to 12-membered heterocycle, each of which is optionally substituted with one or more substituents independently selected from halo, hydroxyl, cyano, nitro, amino, oxo, thioxo, —C(O)OCH₂C₆H₅, —NHC(O)OCH₂C₆H₅, C₁₋₁₀ alkyl, C₁₋₁₀ haloalkyl, C₁₋₁₀ alkoxy, C₂₋₁₀ alkenyl, C₂₋₁₀ alkynyl, C₃₋₁₂ cycloalkyl, and 3- to 12-membered heterocyclyl.

In some embodiments, each L¹, L², or L³ is optionally substituted with one or more substituents selected from alkyl, alkenyl, alkynyl, halo, hydroxyl, cyano, —OR⁸, —SR⁸, amino, aminyl, amido, cycloalkyl, aryl, heterocyclyl, heteroaryl, cycloalkylalkyl, arylalkyl, heterocyclylalkyl, heteroarylalkyl, —C(O)R⁸, —C(O)N(R⁸)₂, —N(R⁸)C(O)R⁸, —C(O)OR⁸, —OC(O)R⁸, —S(O)R⁸, —S(O)₂R⁸, —P(O)(OR⁸)₂, —OP(O)(OR⁸)₂, nitro, oxo, thioxo, ═N(R⁸), or cyano; and
R⁸ is hydrogen, alkyl, alkenyl, alkynyl, cycloalkyl, aryl, heterocyclyl, heteroaryl, cycloalkylalkyl, arylalkyl, heterocyclylalkyl, or heteroarylalkyl.

In some embodiments, L¹, L², or L³ are independently selected from the following structures:

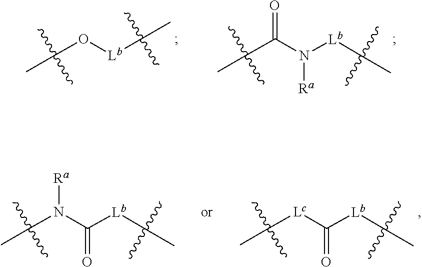

wherein:
R^{a} is hydrogen or alkyl;
each occurrence of L^{b} is independently a direct bond, an optionally substituted alkylene linker, an optionally substituted heteroalkylene linker, a heteroatomic linker, or a combination thereof;
each occurrence of L^{c} is independently an optionally substituted alkylene linker; provided that at least one of L¹, L², or L³ has the following structure:

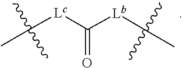

In some embodiments, L¹ and L² are independently selected from the following structures:

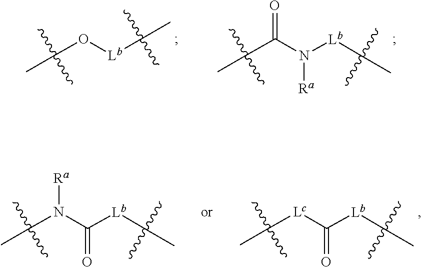

wherein:
R^{a} is hydrogen or alkyl;
each occurrence of L^{b} is independently a direct bond, an optionally substituted alkylene linker, an optionally substituted heteroalkylene linker, a heteroatomic linker, or a combination thereof;
each occurrence of L^{c} is independently an optionally substituted alkylene linker; provided that at least one of L¹ or L² has the following structure:

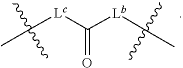

In some embodiments, L² has the following structure:

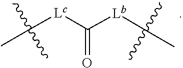

In some embodiments, L¹, L², and L³ each independently have one of the following structures:

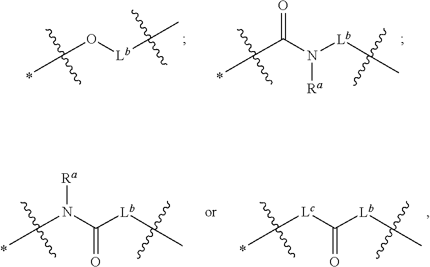

wherein:
* indicates a direct bond to a substitutable position on the phenyl group of following structure:

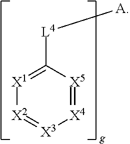

In some embodiments, L¹ or L² has one of the following structures:

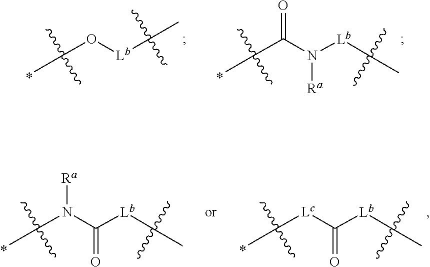

wherein:
* indicates a direct bond to a substitutable position on the phenyl group of following structure:

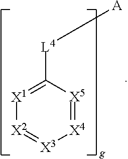

In some embodiments, L^{c} is C₁-C₆ alkylene.

In some embodiments, L^{c} is substituted with one or more substituents selected from the group consisting of halo, haloalkyl, alkoxy, cyano, nitro, carboxy, sulfonamide, sulfonic acid, or combinations thereof. In some embodiments, L^{c} is unsubstituted.

In some embodiments, the conjugate has one of the following Structures (IIa) or (IIb):

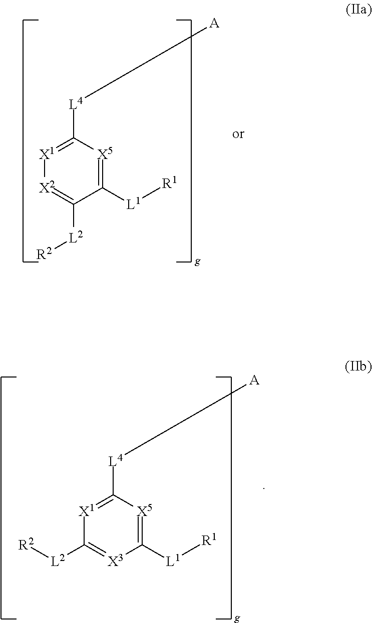

In some embodiments, the conjugate has one of the following Structures (IIc′) or (IIc″):

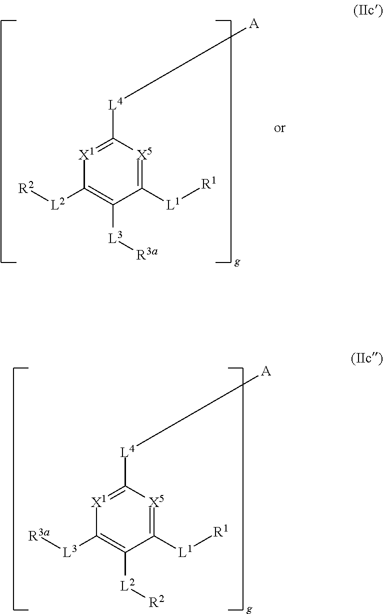

In certain embodiments, X², X³, or both are C—H, or C—F. In some embodiments, X¹, X⁵, or both are C—R³ and R³ is H or halo. In some embodiments, X¹ and X⁵ are both C—R³ and R³ is H or halo. In some embodiments, X¹ is C—R³ and R³ is halo. In certain embodiments, X⁵ is C—R³ and R³ is halo. In some embodiments, halo is fluoro. In some embodiments, X¹ is C—F. In some embodiments, X¹ is C—H. In some embodiments, X⁵ is C—H. In some embodiments, X⁵ is C—F. In some embodiments, X³ is C—R³ and R³ is H. In some embodiments, X³ is C—R³ and R³ is halo (e.g., fluoro).

In some embodiments, the conjugate has one of the following structures (IIa-1), (IIa-2), (IIa-3), (IIa-4), (IIa-5), or (IIa-6):

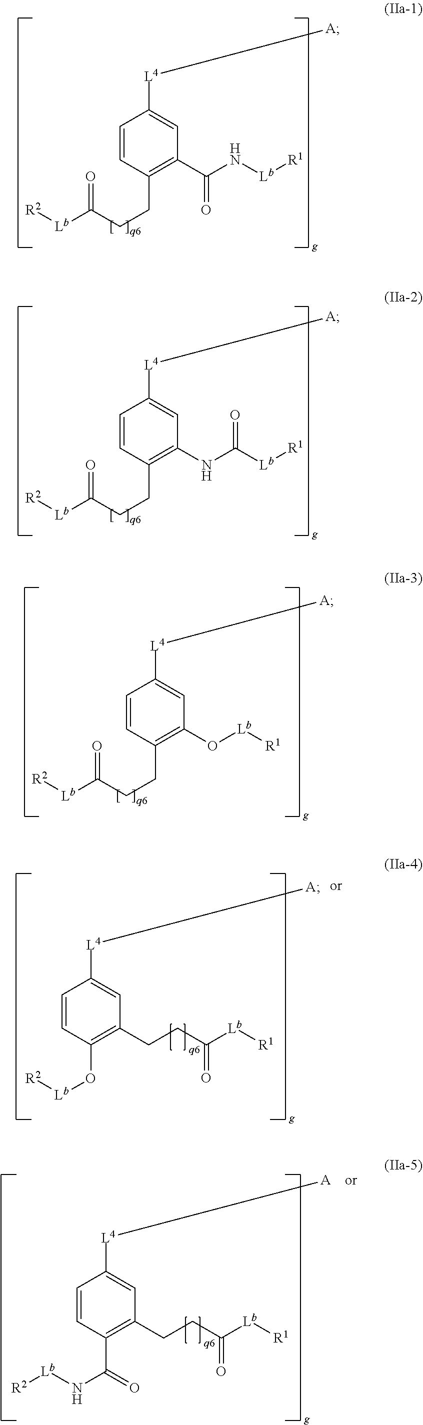

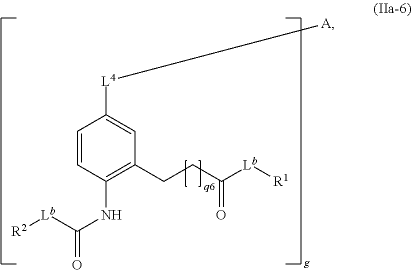

wherein:
each occurrence of L^{b} is independently a direct bond, an optionally substituted alkylene linker, an optionally substituted heteroalkylene linker, a heteroatomic linker, or combinations thereof; and
q6 is 0, 1, or 2.

In some embodiments, the conjugate has one of the following structures (IIa-1), (IIa-2), (IIa-3), (IIa-4), (IIa-5), (IIa-6), (IIa-7), or (IIa-8):

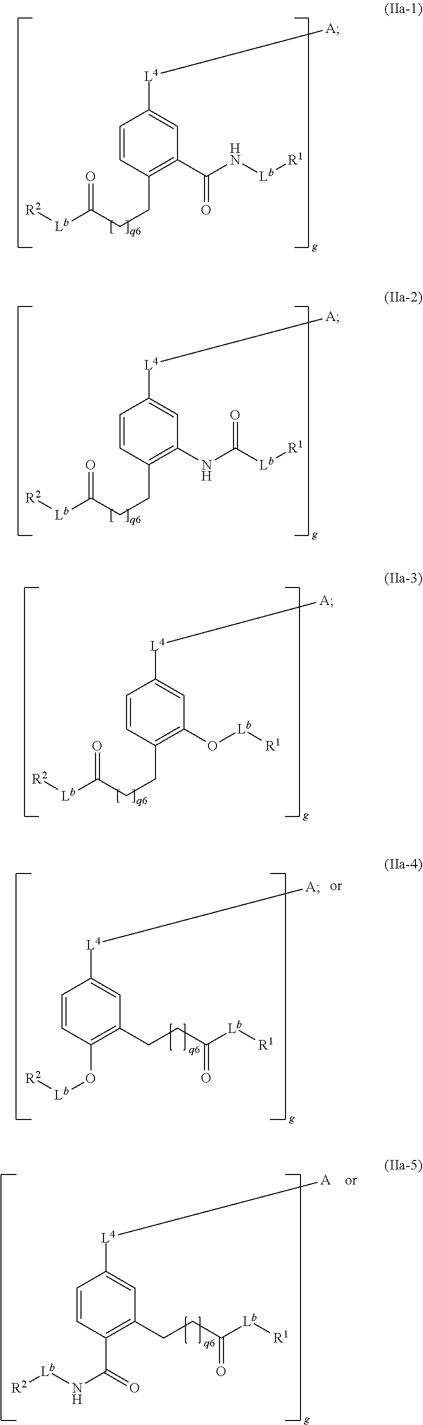

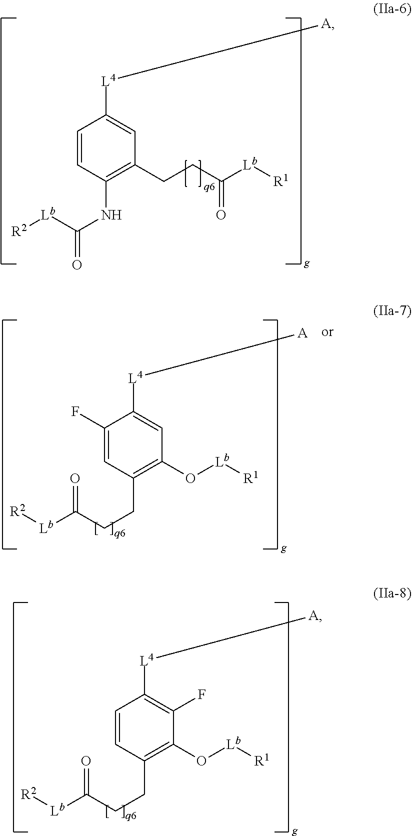

wherein:
each occurrence of L^{b} is independently a direct bond, an optionally substituted alkylene linker, an optionally substituted heteroalkylene linker, a heteroatomic linker, or combinations thereof; and
q6 is 0, 1, or 2.

In certain embodiments, the conjugate has one of the following Structures (IIc-1), (IIc-2), (IIc-3), or (IIc-4):

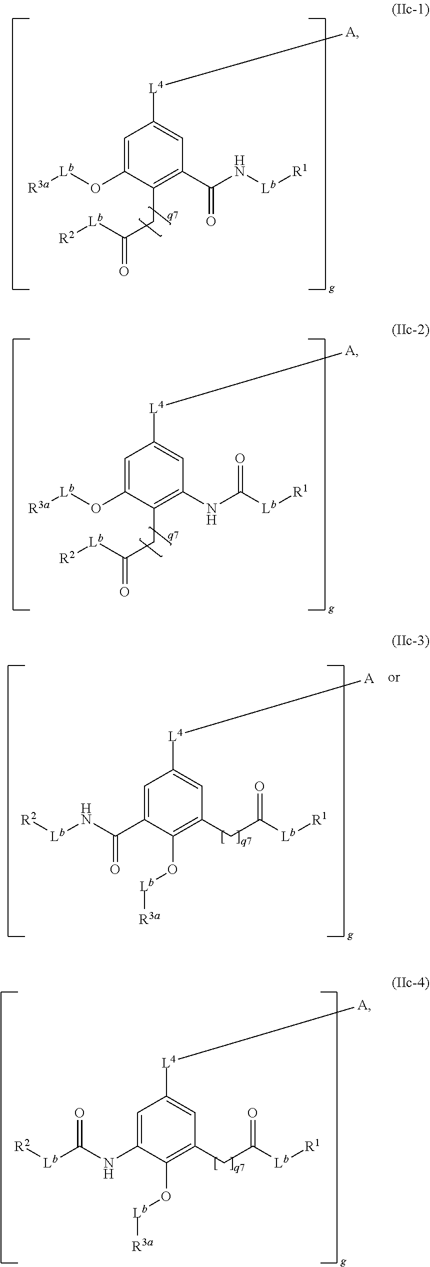

wherein:
each occurrence of L^{b} is independently a direct bond, an optionally substituted alkylene linker, an optionally substituted heteroalkylene linker, a heteroatomic linker, or combinations thereof; and
q7 is 1, 2, or 3.

In some embodiments, the conjugate has the following Structure (IId), (IIe), (IIf), or (IIg):

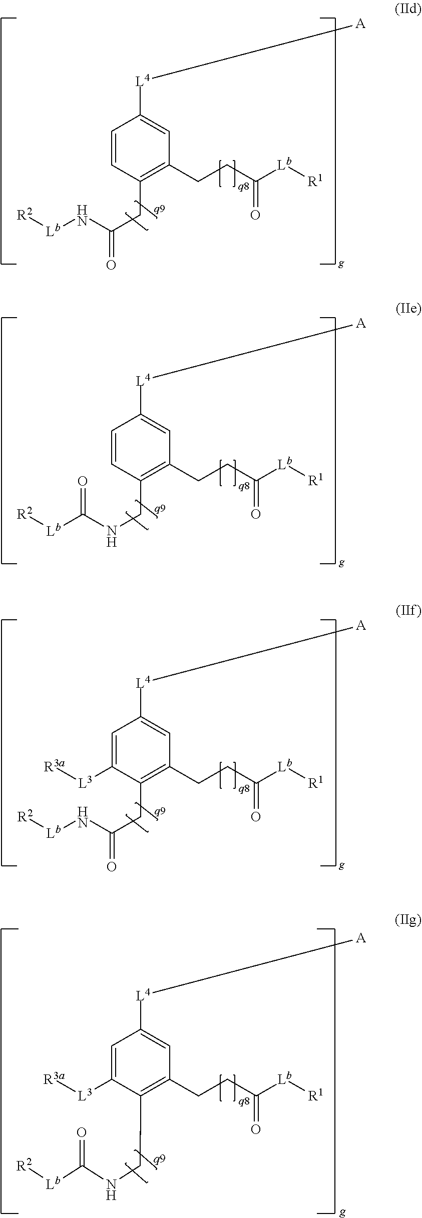

wherein:
each occurrence of L^{b} is independently a direct bond, an optionally substituted alkylene linker, an optionally substituted heteroalkylene linker, a heteroatomic linker, or combinations thereof;
q8 is 0, 1, or 2; and
q9 is 0, 1, or 2.

In some embodiments, the conjugate has one of the following Structures (IIh) or (IIi):

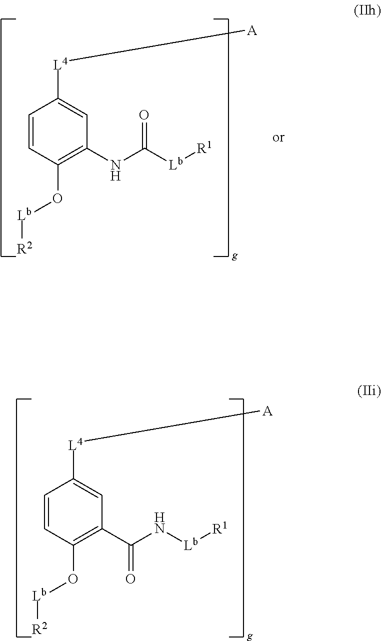

wherein:
each occurrence of L^{b} is independently a direct bond, an optionally substituted alkylene linker, an optionally substituted heteroalkylene linker, a heteroatomic linker, or combinations thereof.

In some embodiments, L^{b} is a direct bond, an optionally substituted alkylene linker or an optionally substituted heteroalkylene linker.

In certain embodiments, L^{b} is a direct bond or has one of the following structures:

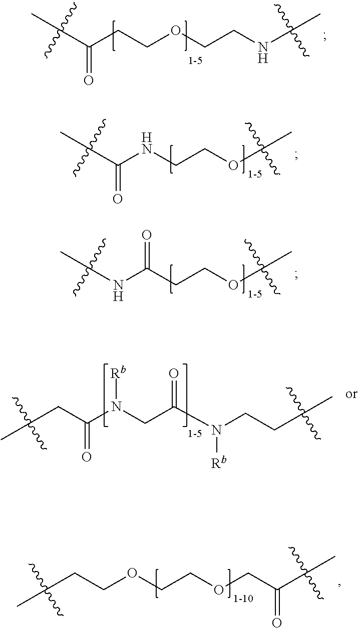

wherein:
each occurrence of R^{b} is independently hydrogen, alkyl, hydroxyalkyl, or alkoxyalkyl.

In some embodiments, the conjugate has one of the following structures:

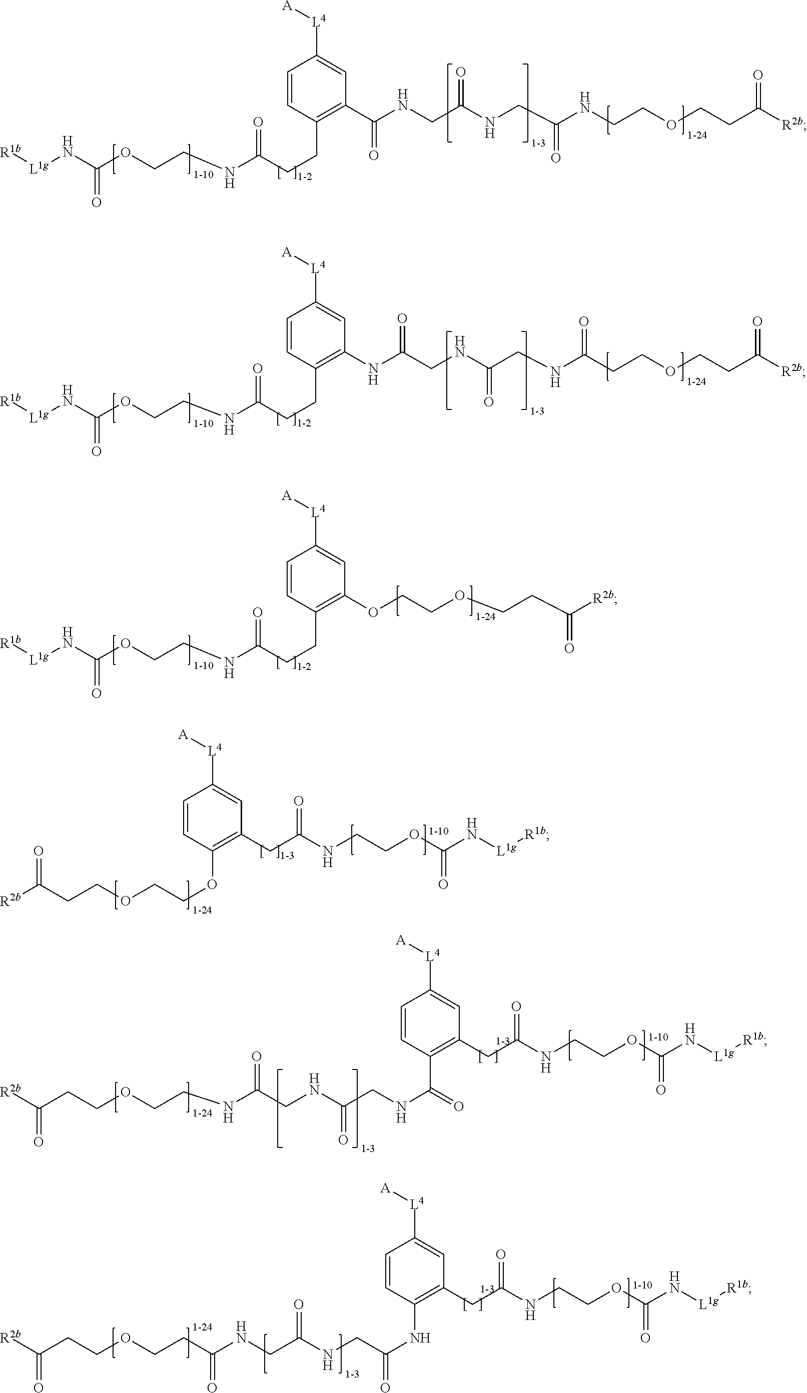

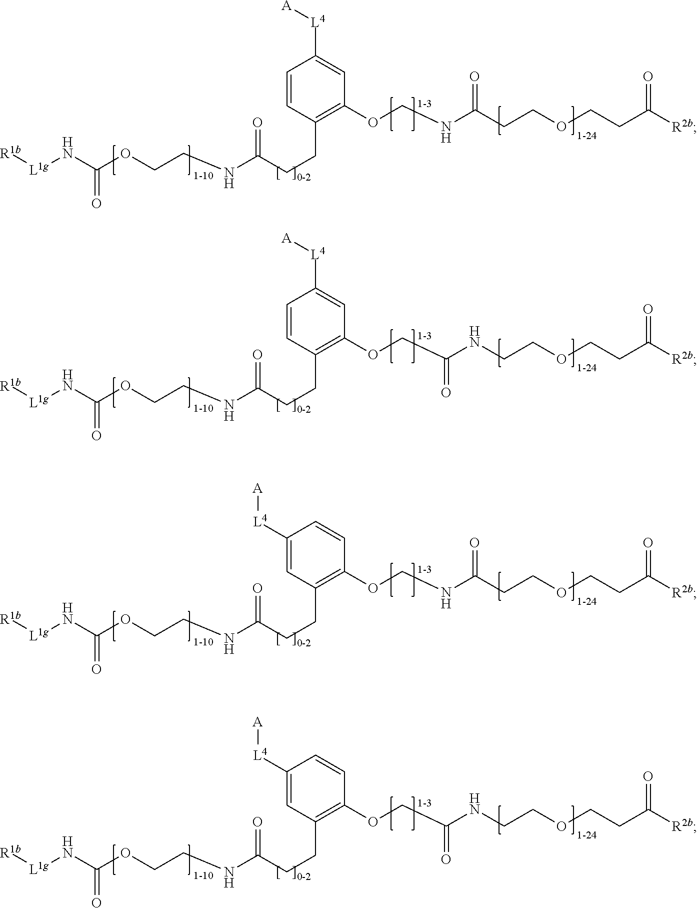

wherein:
R^{1b} is a chemotherapeutic, a cytotoxic agent, or a myeloid cell agonist;
R^{2b} has one of the following structures:

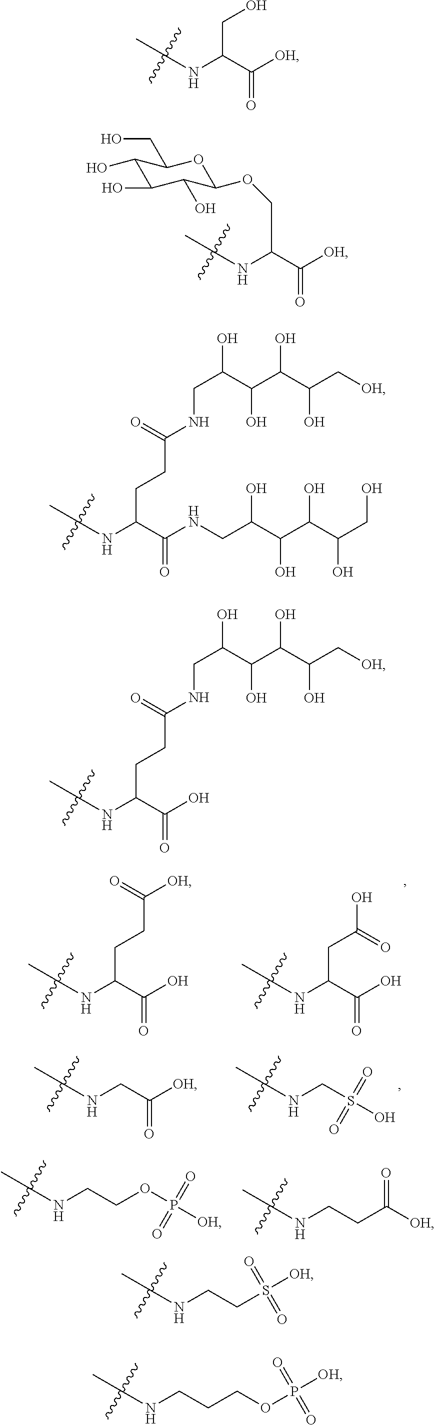

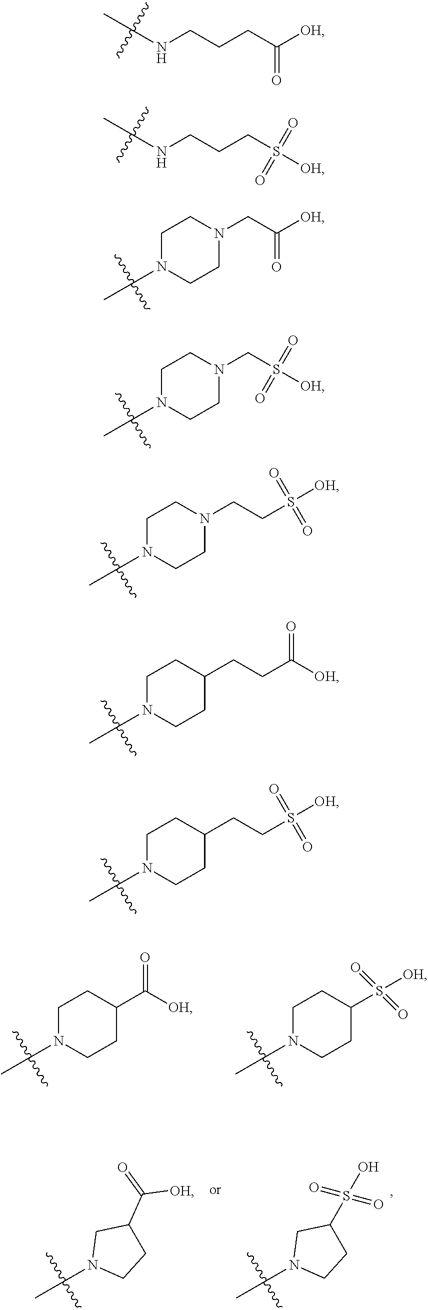

L^{1g} has one of the following structures:

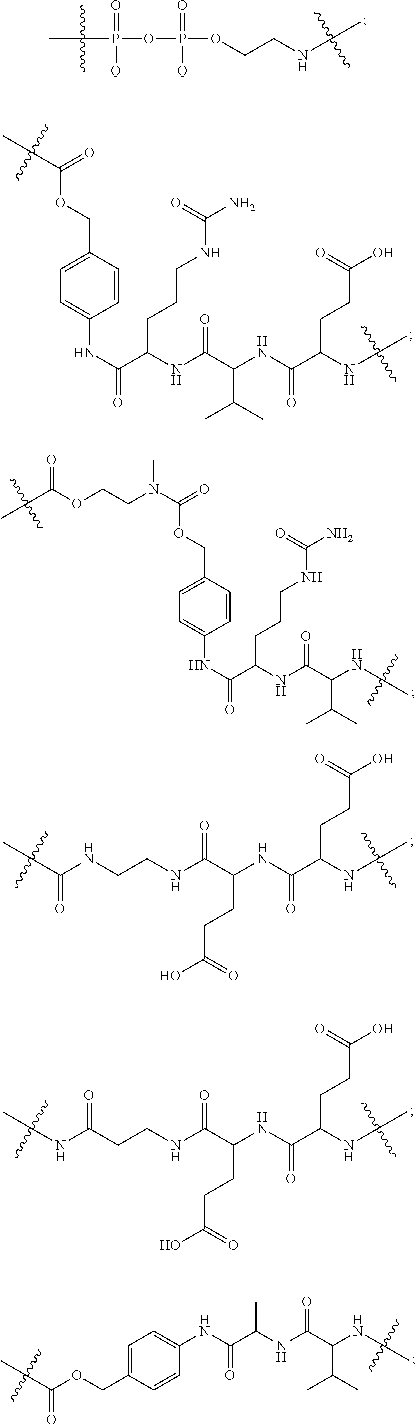

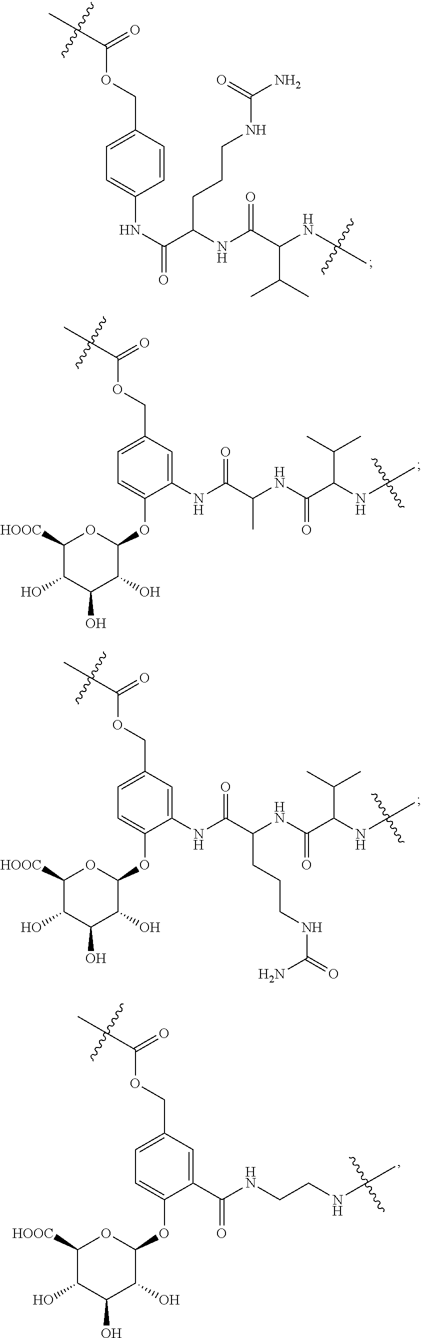 [SMILES: *NC(C(=O)NC(CCCNC(N)=O)C(=O)Nc1ccc(COC(*)=O)cc1)C(C)C ; *NC(C(=O)NC(C)C(=O)Nc1cc(COC(*)=O)ccc1O[C@@H]1O[C@H](C(=O)O)[C@@H](O)[C@H](O)[C@H]1O)C(C)C ; *NC(C(=O)NC(CCCNC(N)=O)C(=O)Nc1cc(COC(*)=O)ccc1O[C@@H]1O[C@H](C(=O)O)[C@@H](O)[C@H](O)[C@H]1O)C(C)C ; *NCCNC(=O)c1cc(COC(*)=O)ccc1O[C@@H]1O[C@H](C(=O)O)[C@@H](O)[C@H](O)[C@H]1O]

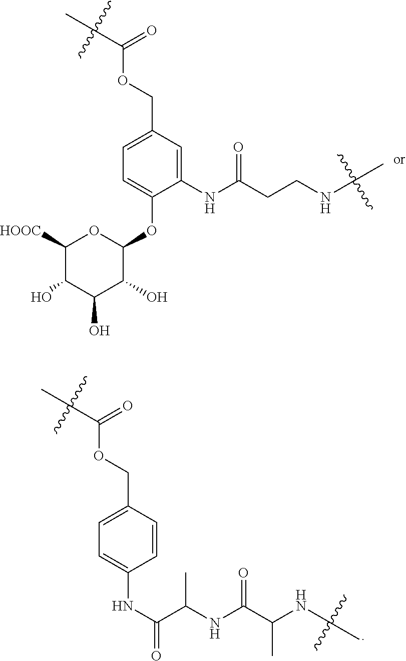 [SMILES: *NCCC(=O)Nc1cc(COC(*)=O)ccc1O[C@@H]1O[C@H](C(=O)O)[C@@H](O)[C@H](O)[C@H]1O ; *NC(C)C(=O)NC(C)C(=O)Nc1ccc(COC(*)=O)cc1]

In some embodiments, the conjugate has one of the following structures:

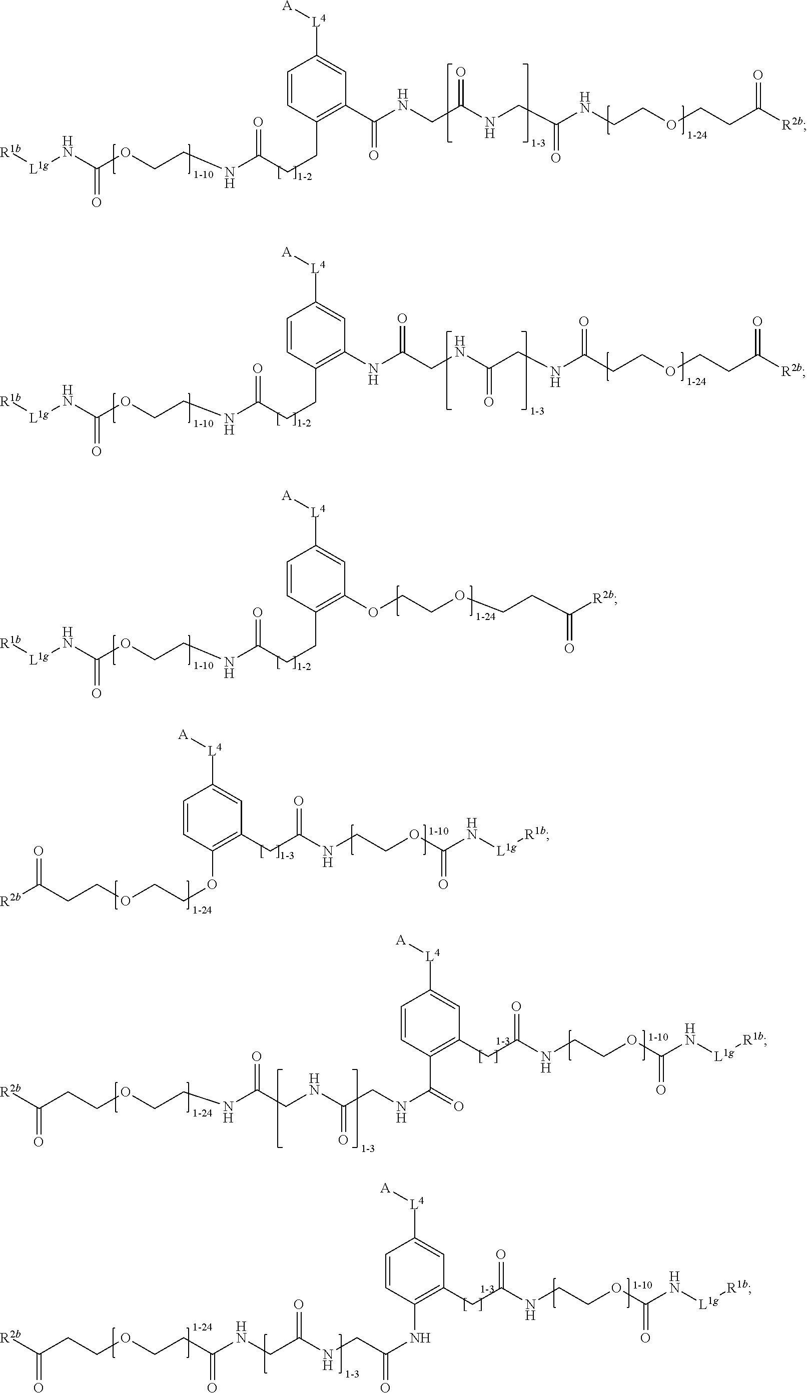

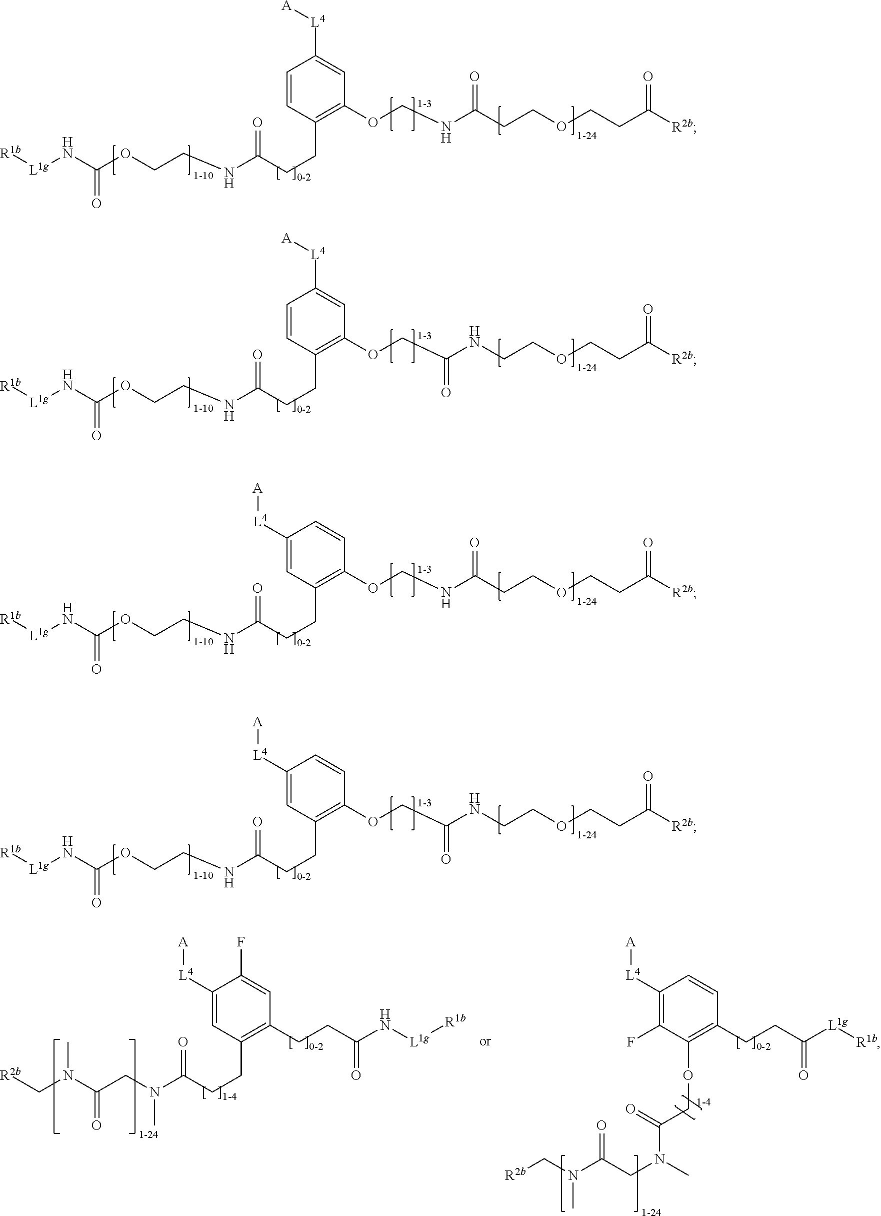

wherein:
R^{1b} is a chemotherapeutic, a cytotoxic agent, or a myeloid cell agonist;
R^{2b} has one of the following structures:

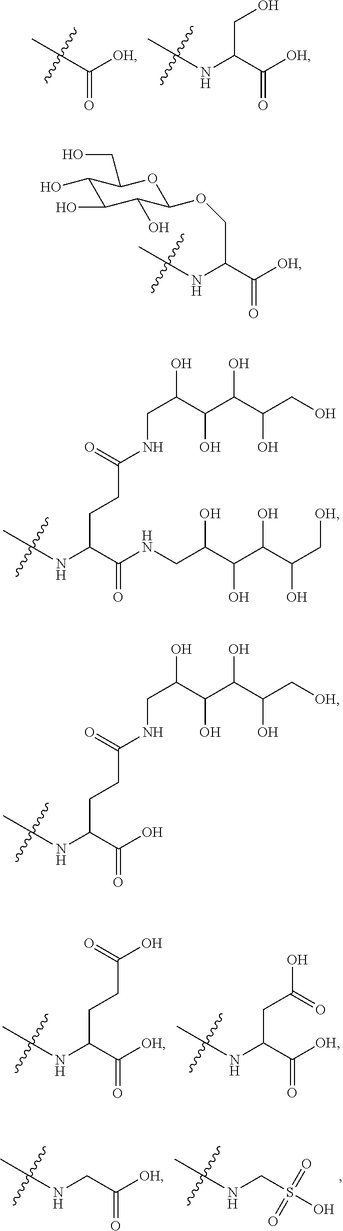

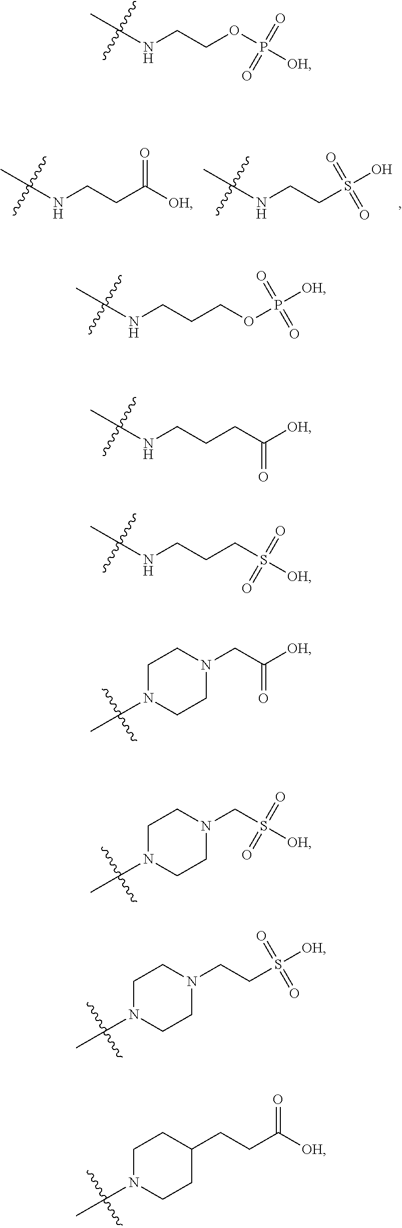

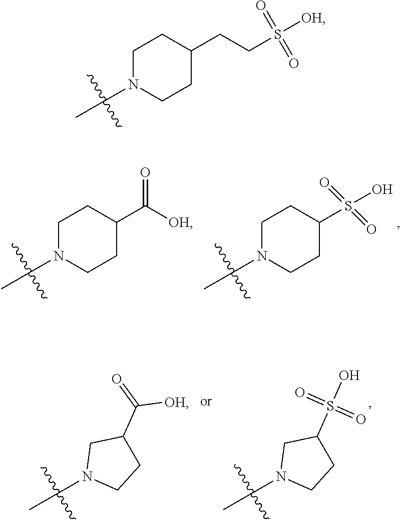 [SMILES: *N1CCC(CCS(=O)(=O)O)CC1 ; *N1CCC(C(=O)O)CC1 ; *N1CCC(S(=O)(=O)O)CC1 ; *N1CCC(C(=O)O)C1 ; "or" ; *N1CCC(S(=O)(=O)O)C1]

L^{1g} has one of the following structures:

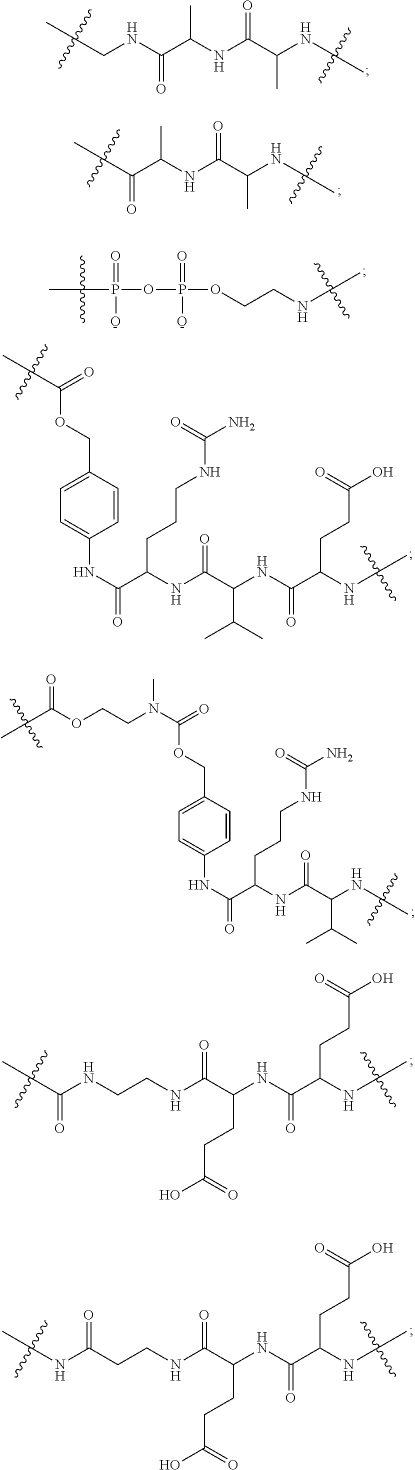

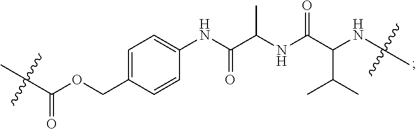 [SMILES: *NC(C(=O)NC(C)C(=O)Nc1ccc(COC(*)=O)cc1)C(C)C]

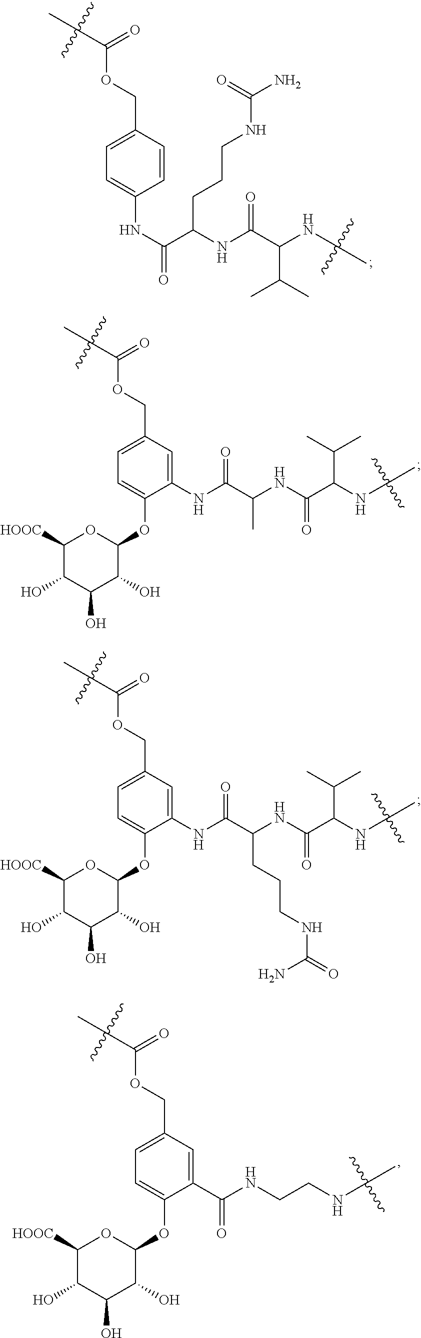 [SMILES: *NC(C(=O)NC(CCCNC(N)=O)C(=O)Nc1ccc(COC(*)=O)cc1)C(C)C ; *NC(C(=O)NC(C)C(=O)Nc1cc(COC(*)=O)ccc1O[C@@H]1O[C@H](C(=O)O)[C@@H](O)[C@H](O)[C@H]1O)C(C)C ; *NC(C(=O)NC(CCCNC(N)=O)C(=O)Nc1cc(COC(*)=O)ccc1O[C@@H]1O[C@H](C(=O)O)[C@@H](O)[C@H](O)[C@H]1O)C(C)C ; *NCCNC(=O)c1cc(COC(*)=O)ccc1O[C@@H]1O[C@H](C(=O)O)[C@@H](O)[C@H](O)[C@H]1O]

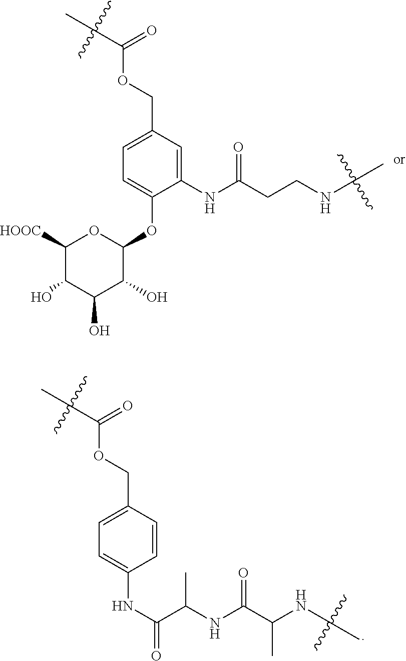 [SMILES: *NCCC(=O)Nc1cc(COC(*)=O)ccc1O[C@@H]1O[C@H](C(=O)O)[C@@H](O)[C@H](O)[C@H]1O ; *NC(C)C(=O)NC(C)C(=O)Nc1ccc(COC(*)=O)cc1]

In certain embodiments, the conjugate has one of the following structures:

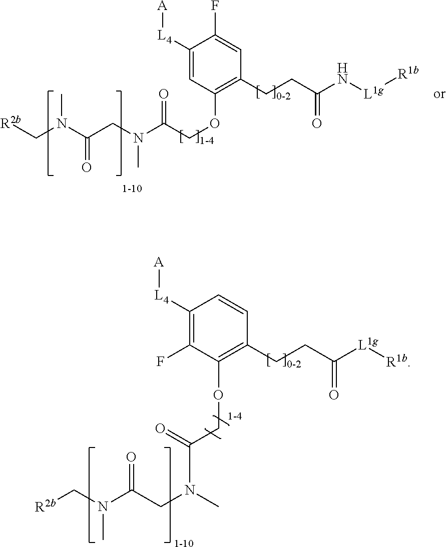

In some embodiments, the conjugate has one of the following structures:

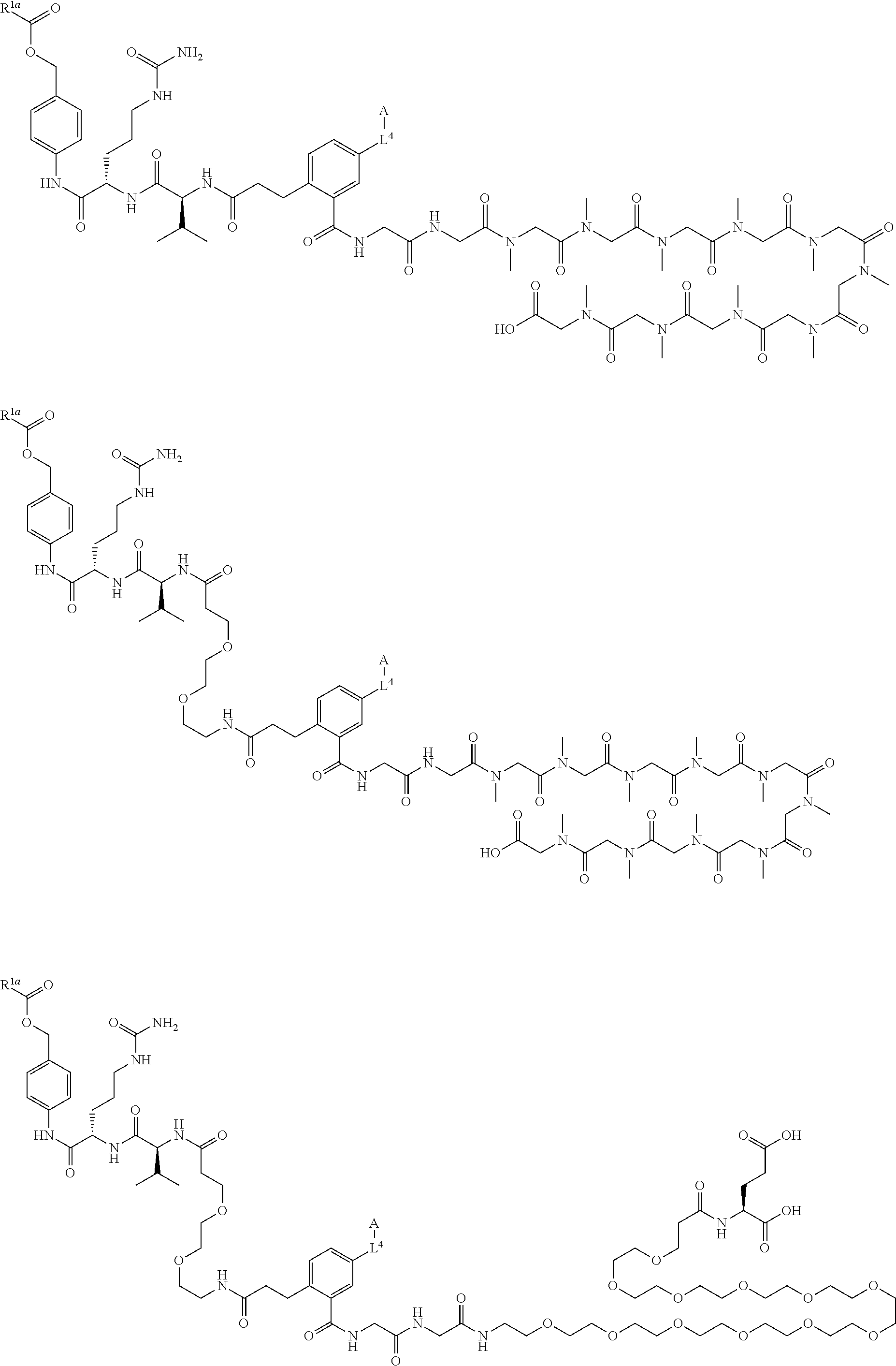

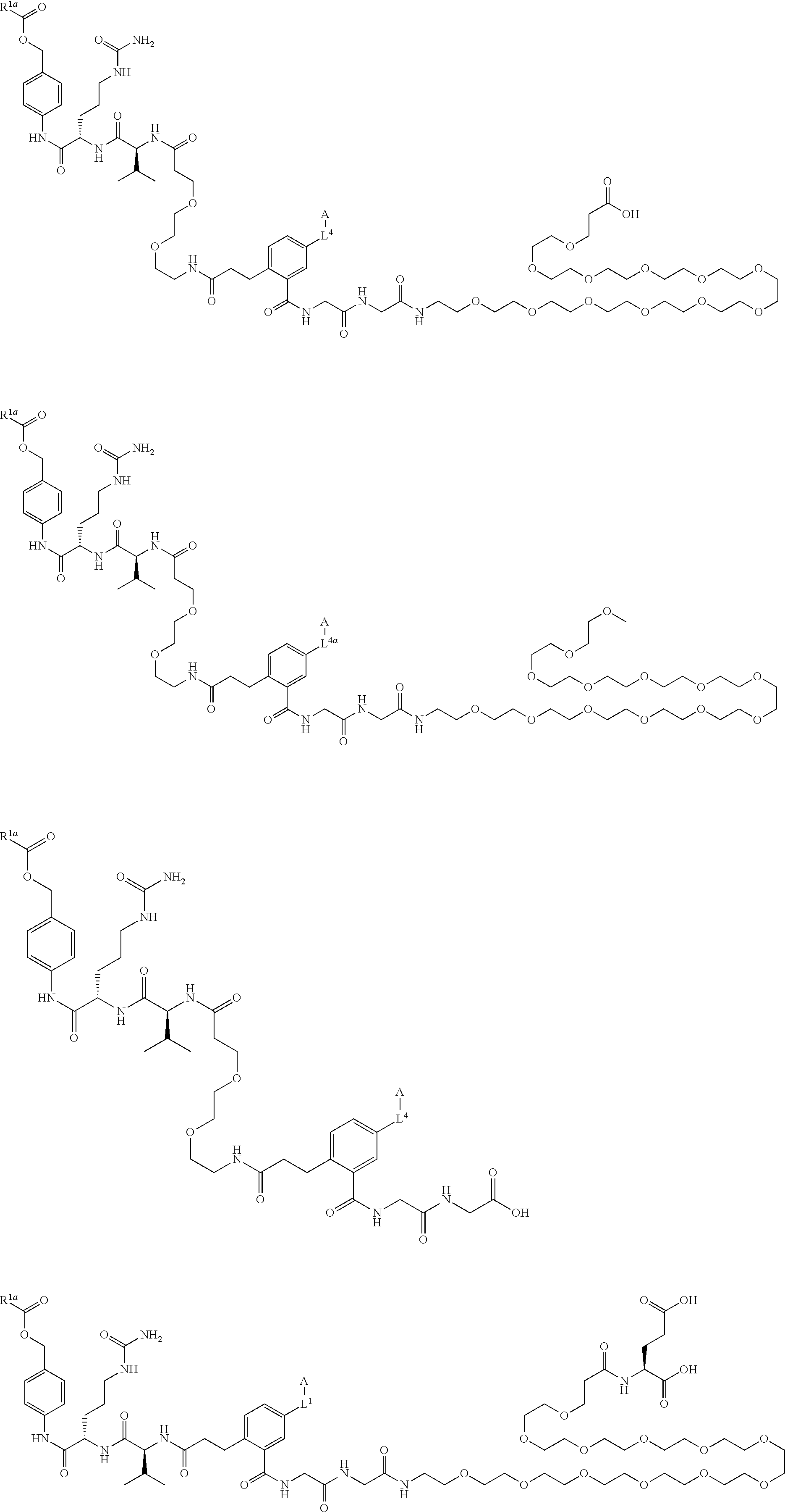

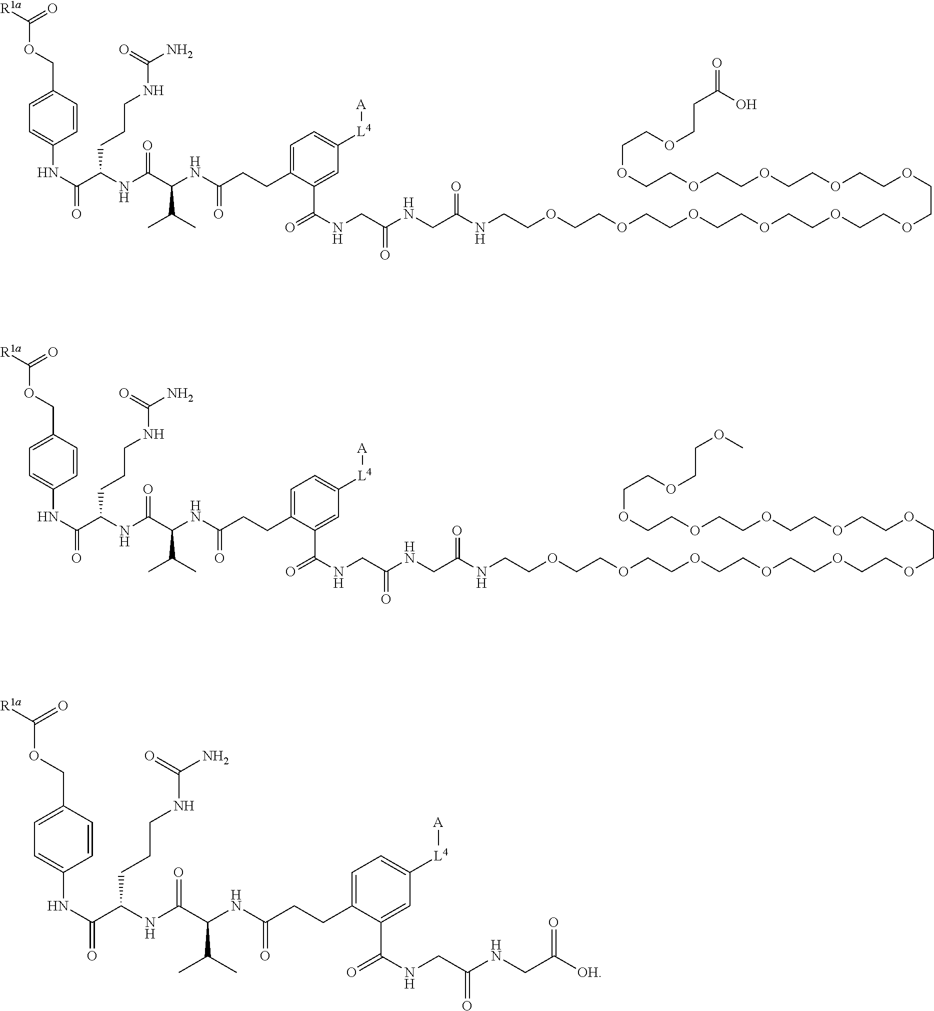

In some embodiments, a conjugate has one of the following structures:

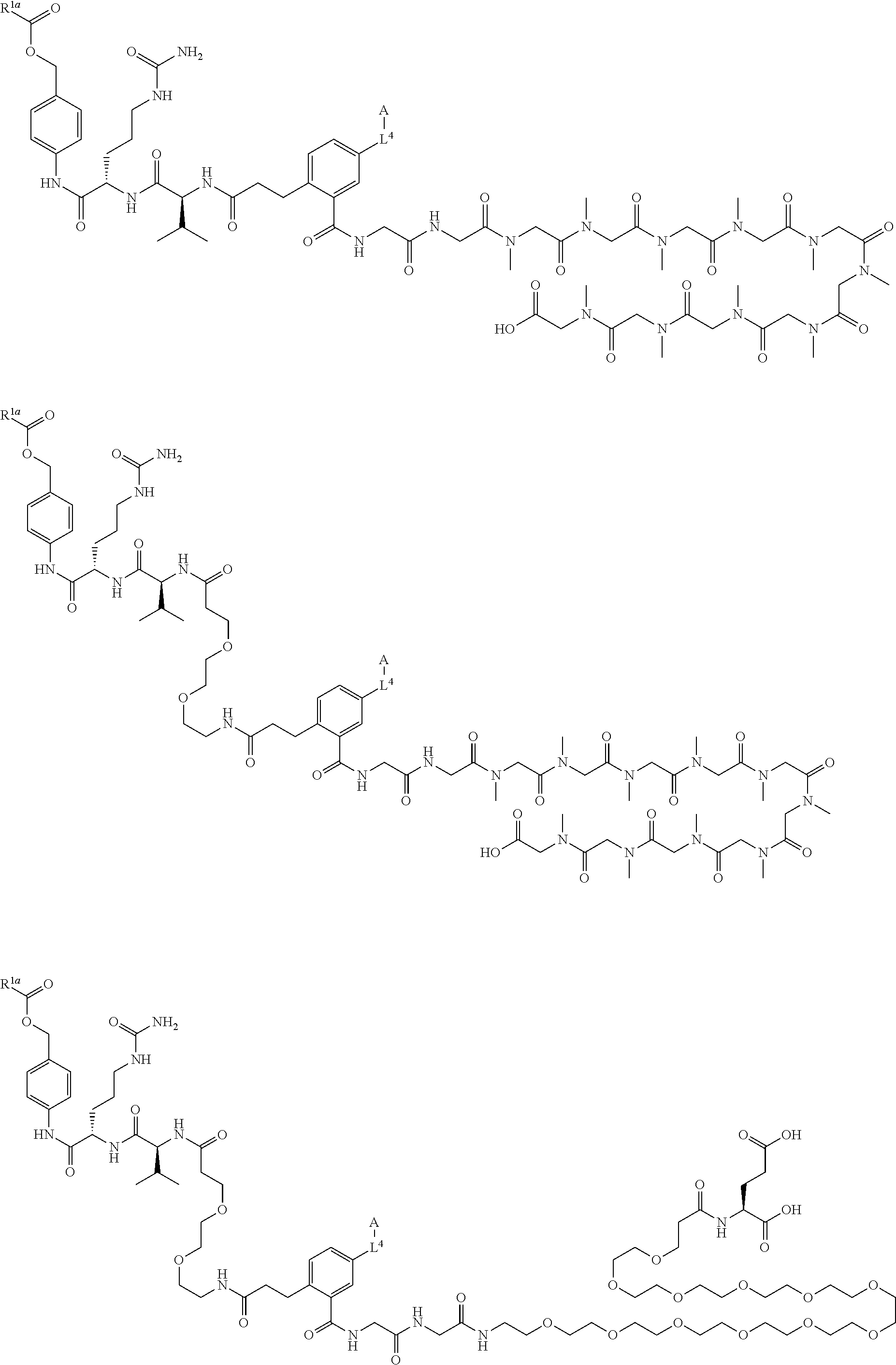

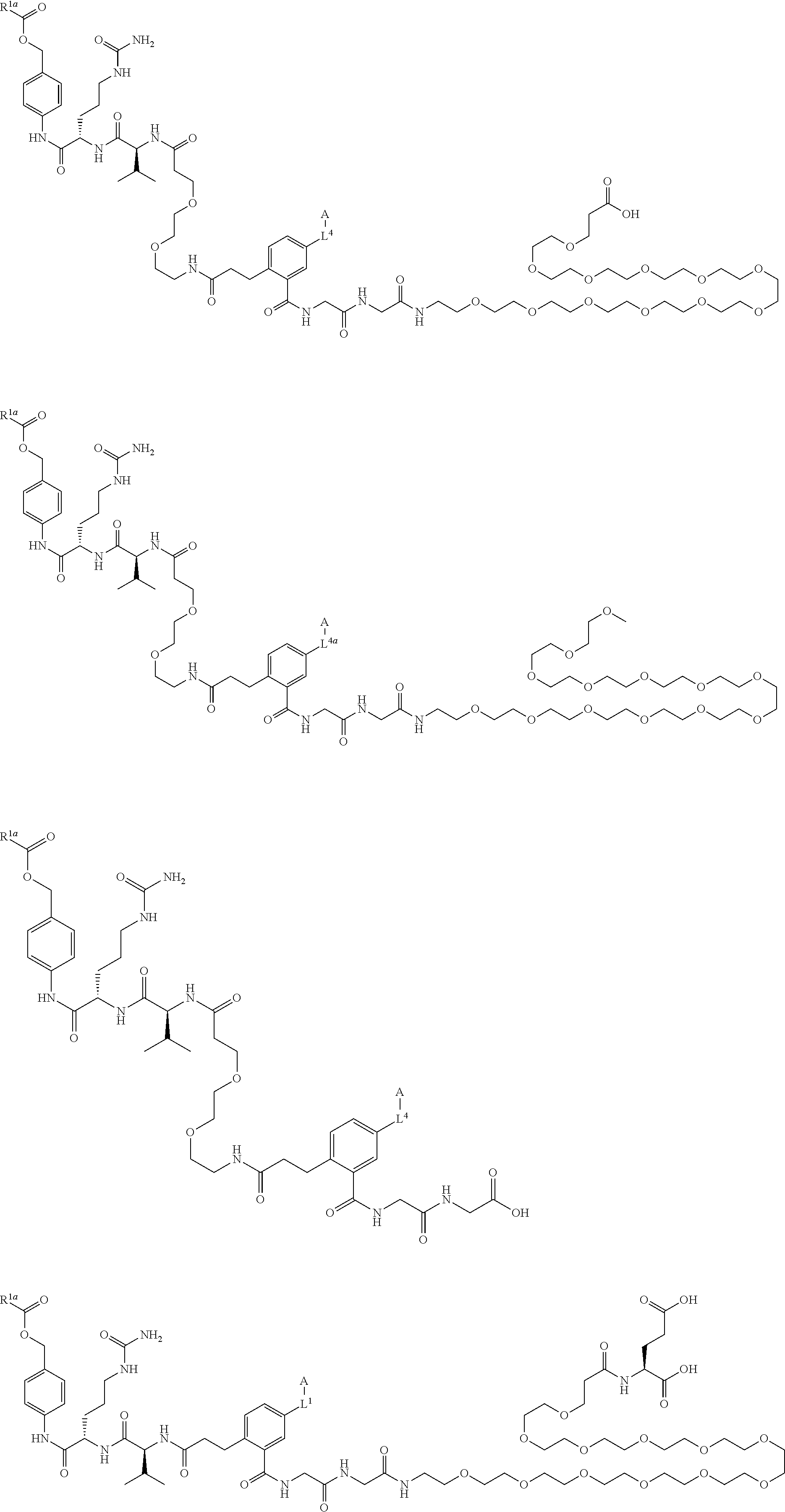

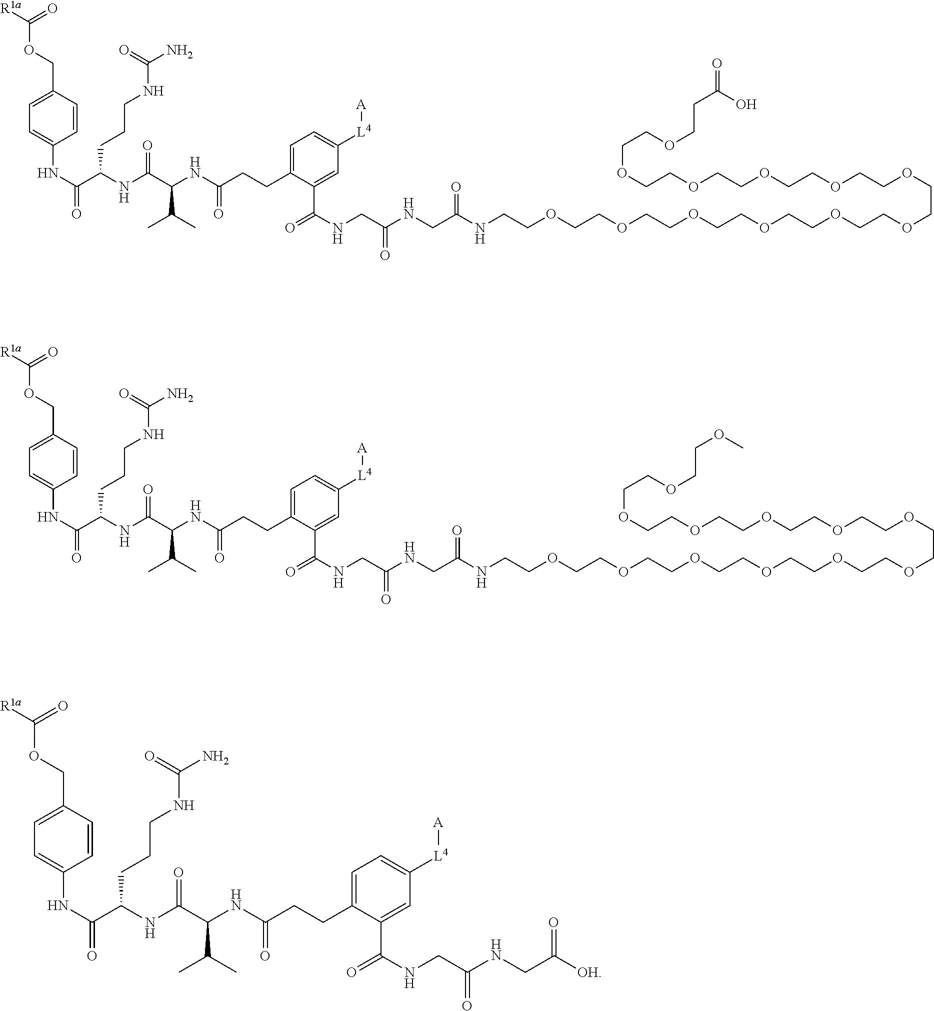

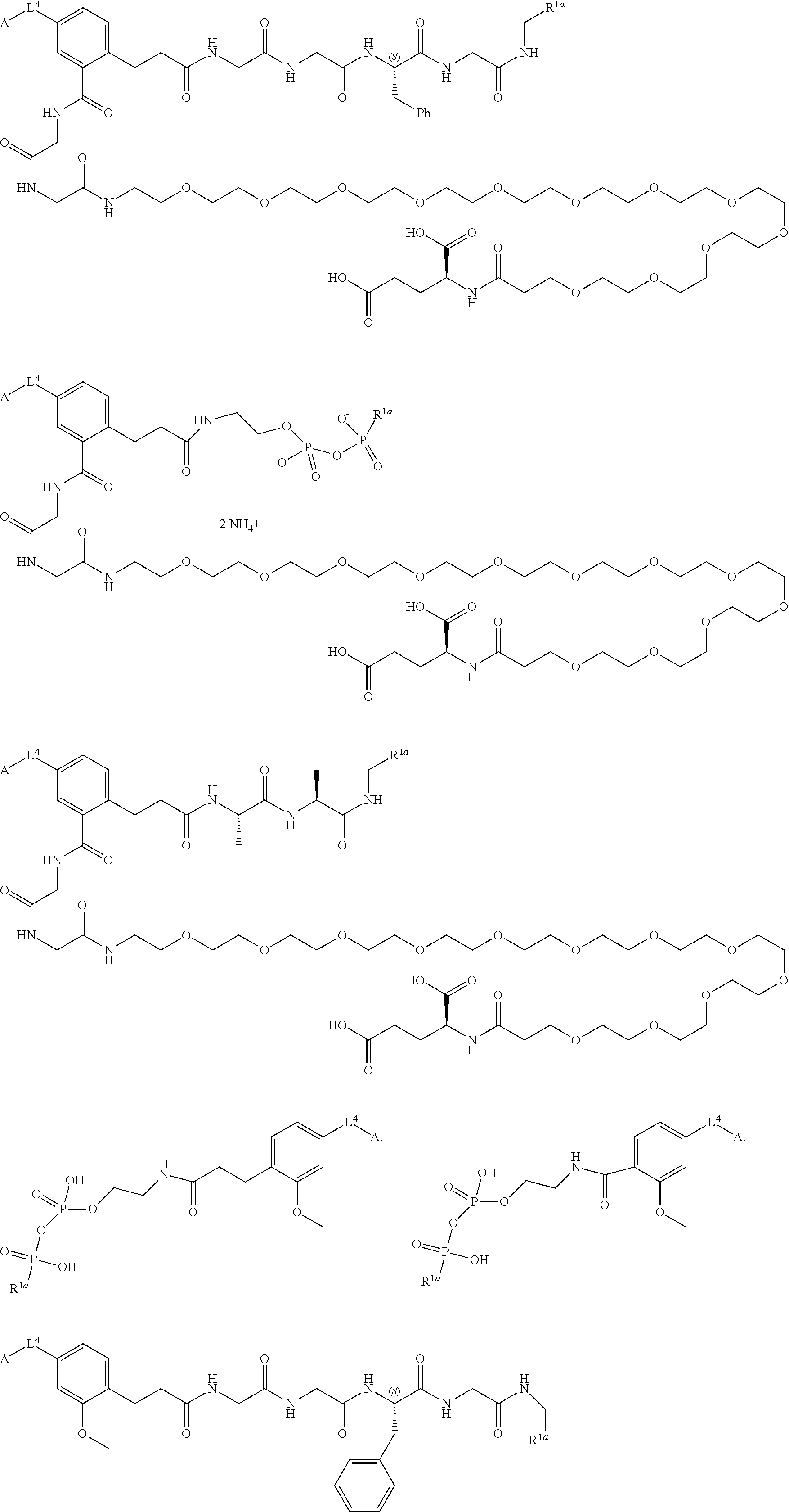

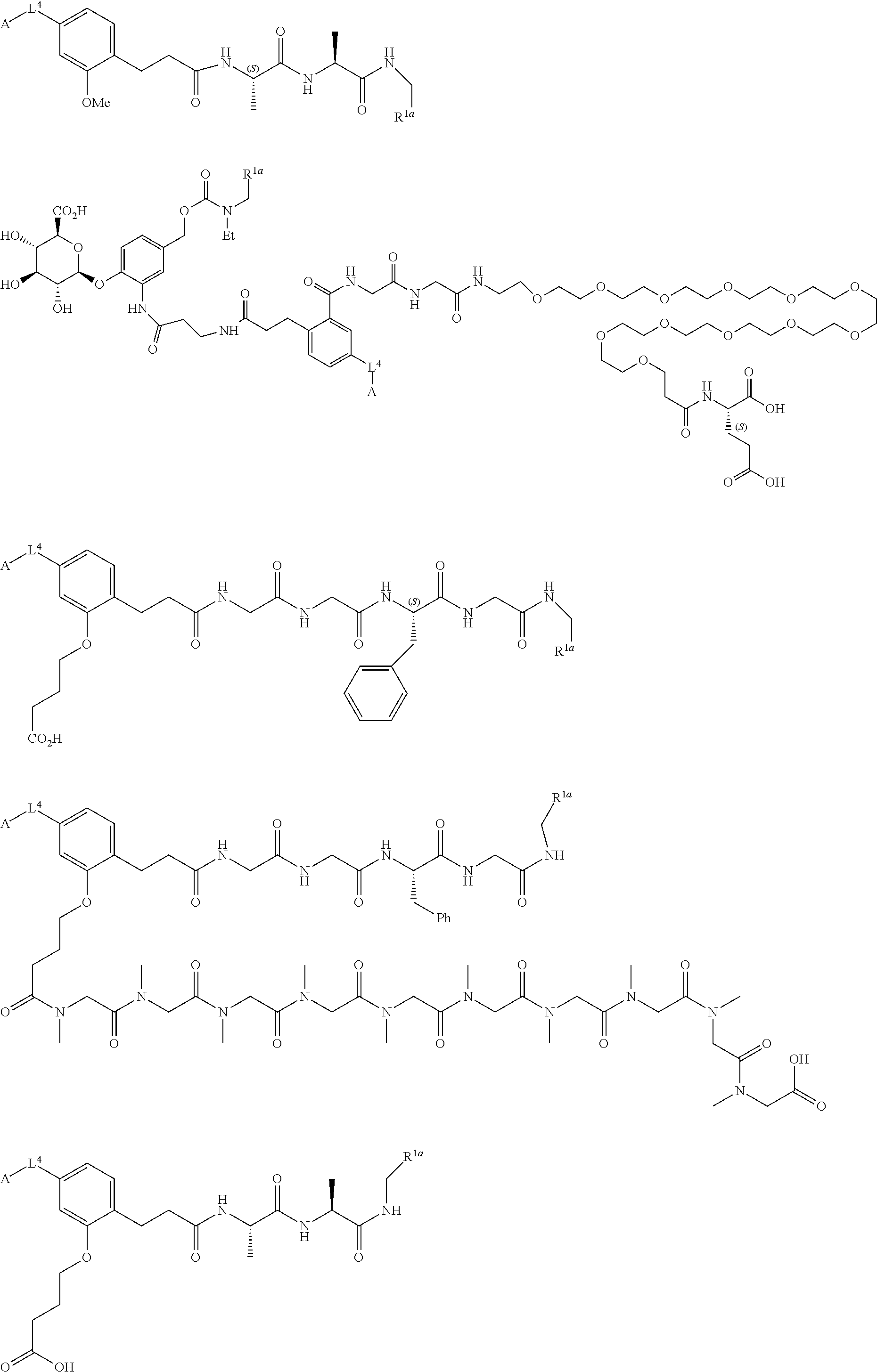

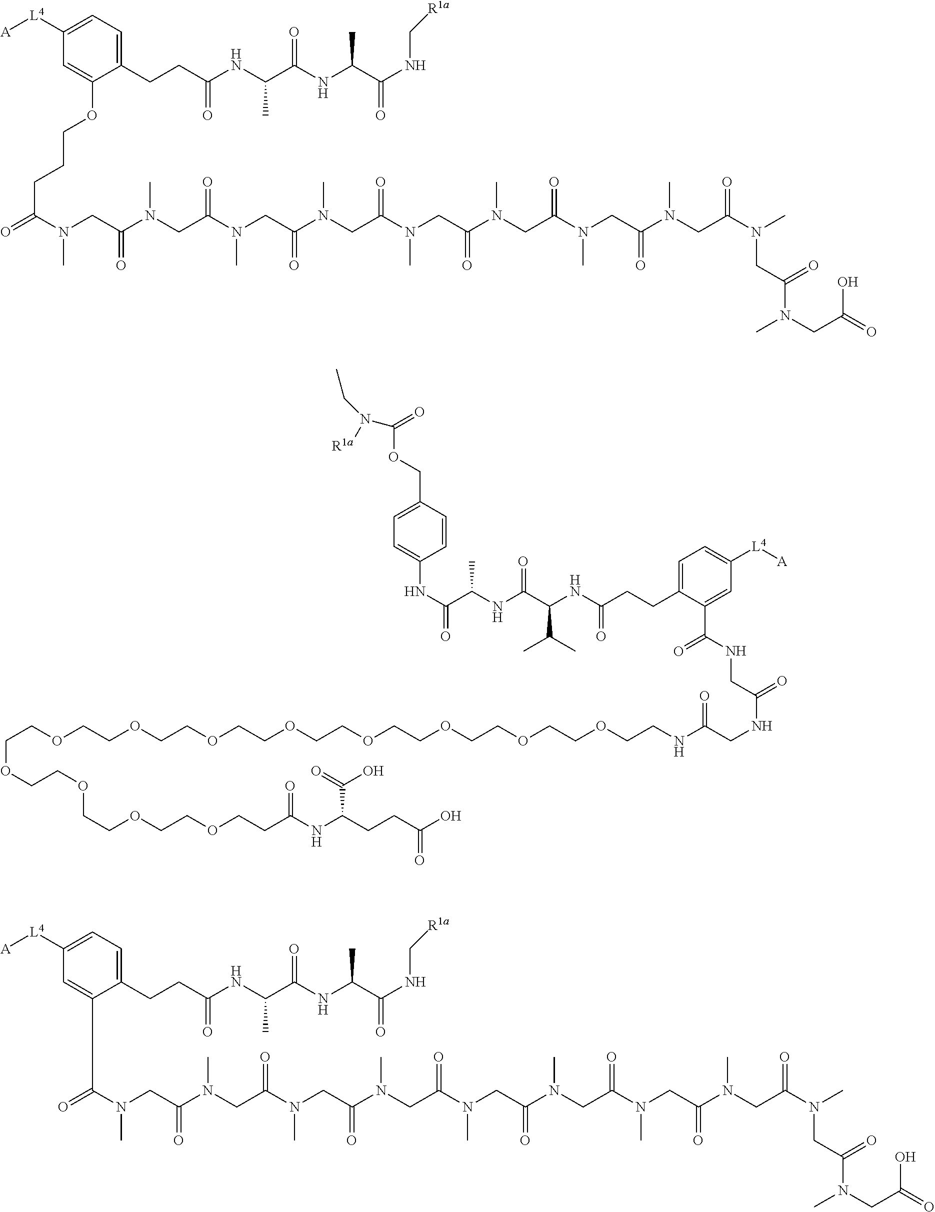

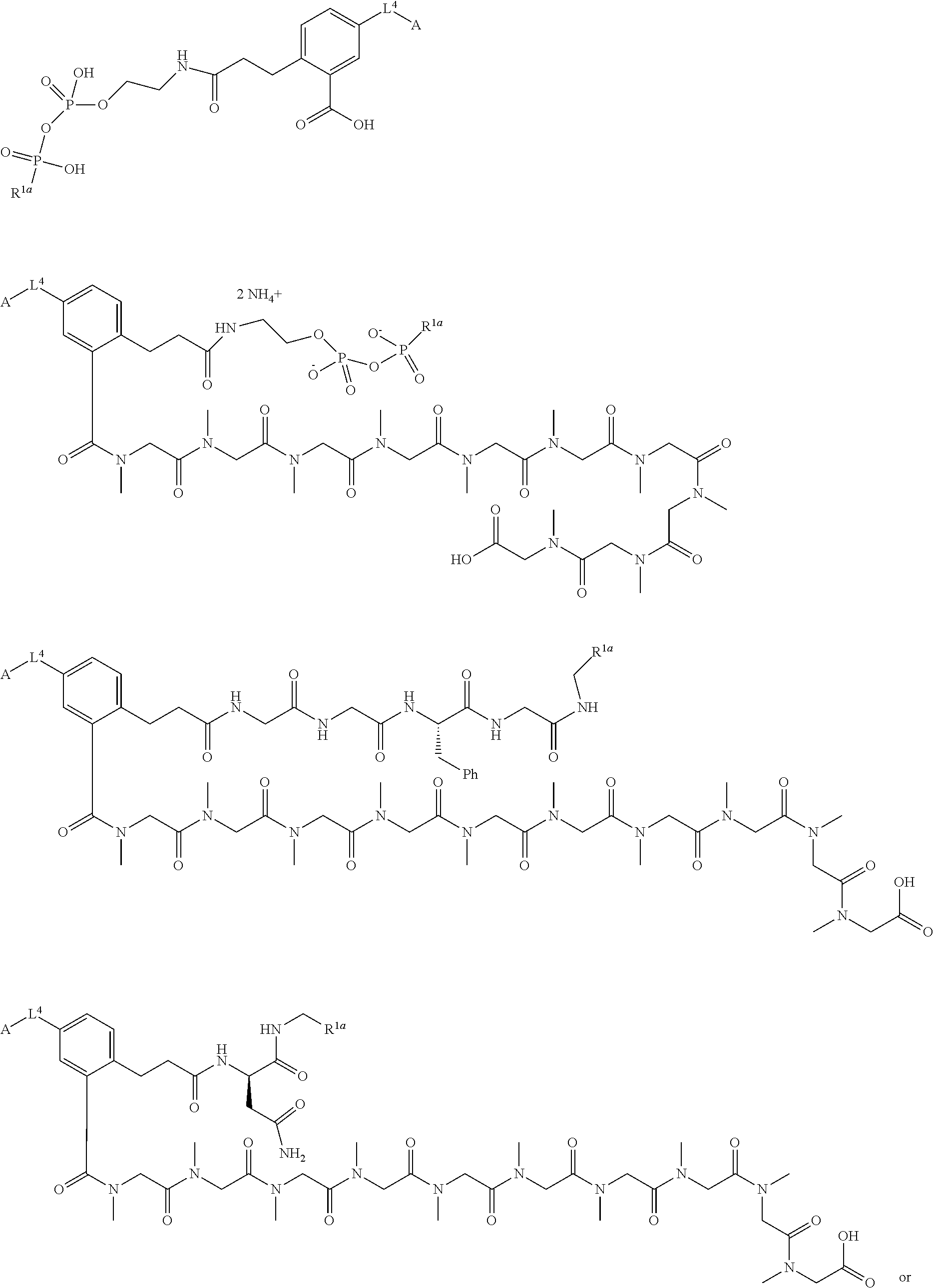

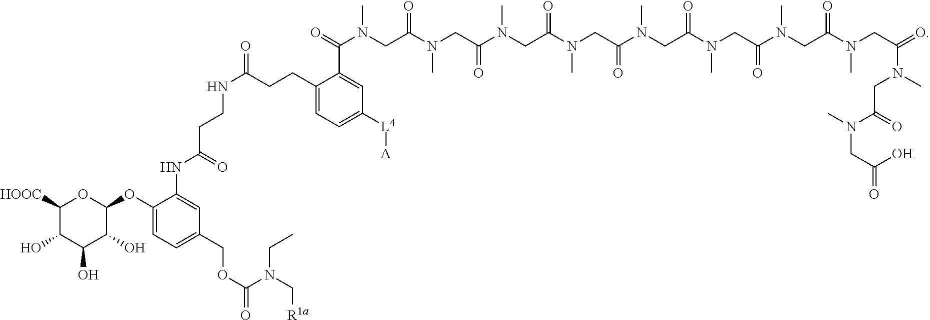

In some embodiments, a conjugate has one of the following structures:

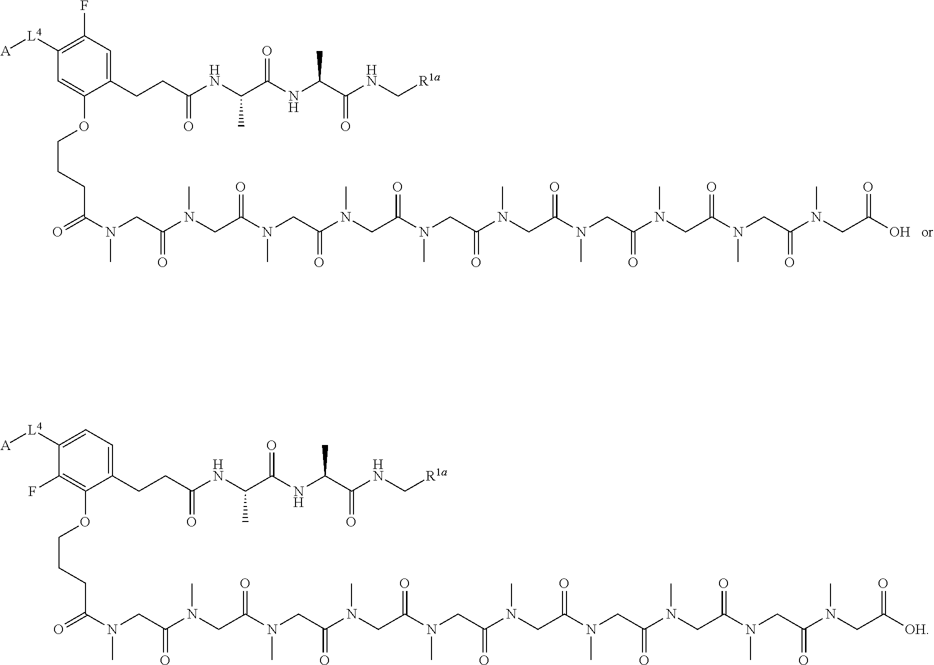

In certain embodiments, a conjugate has one of the following structures:

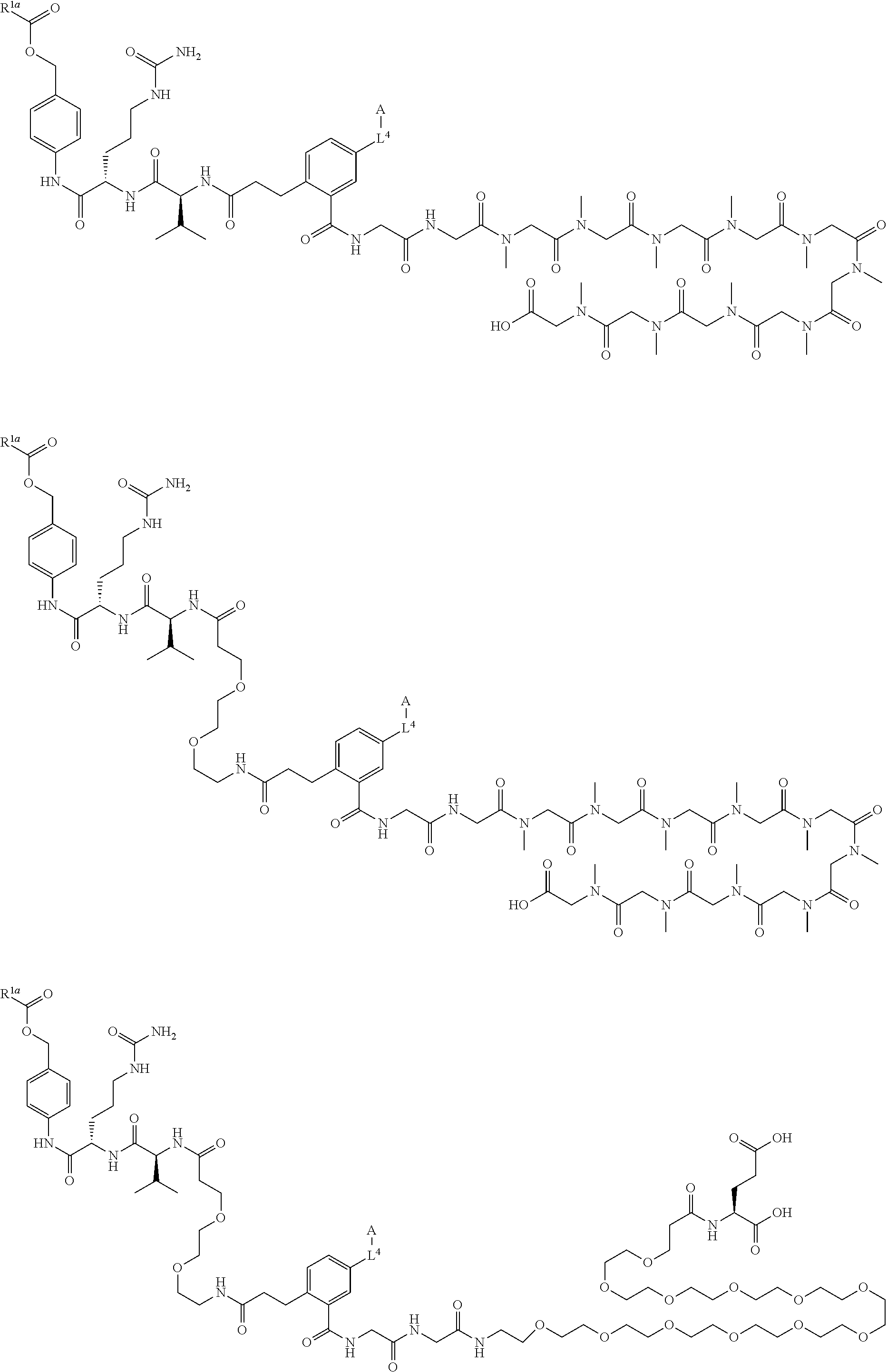

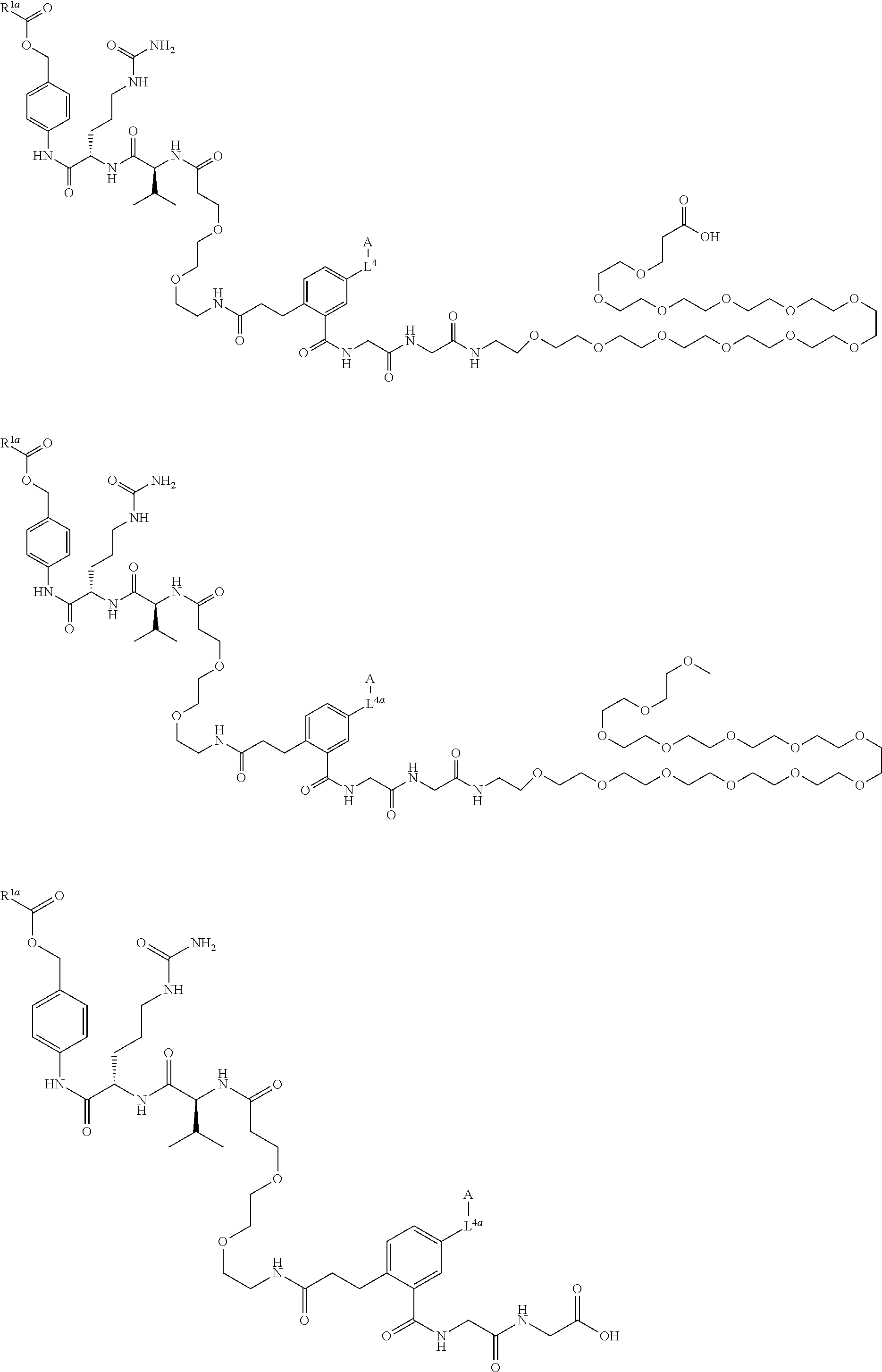

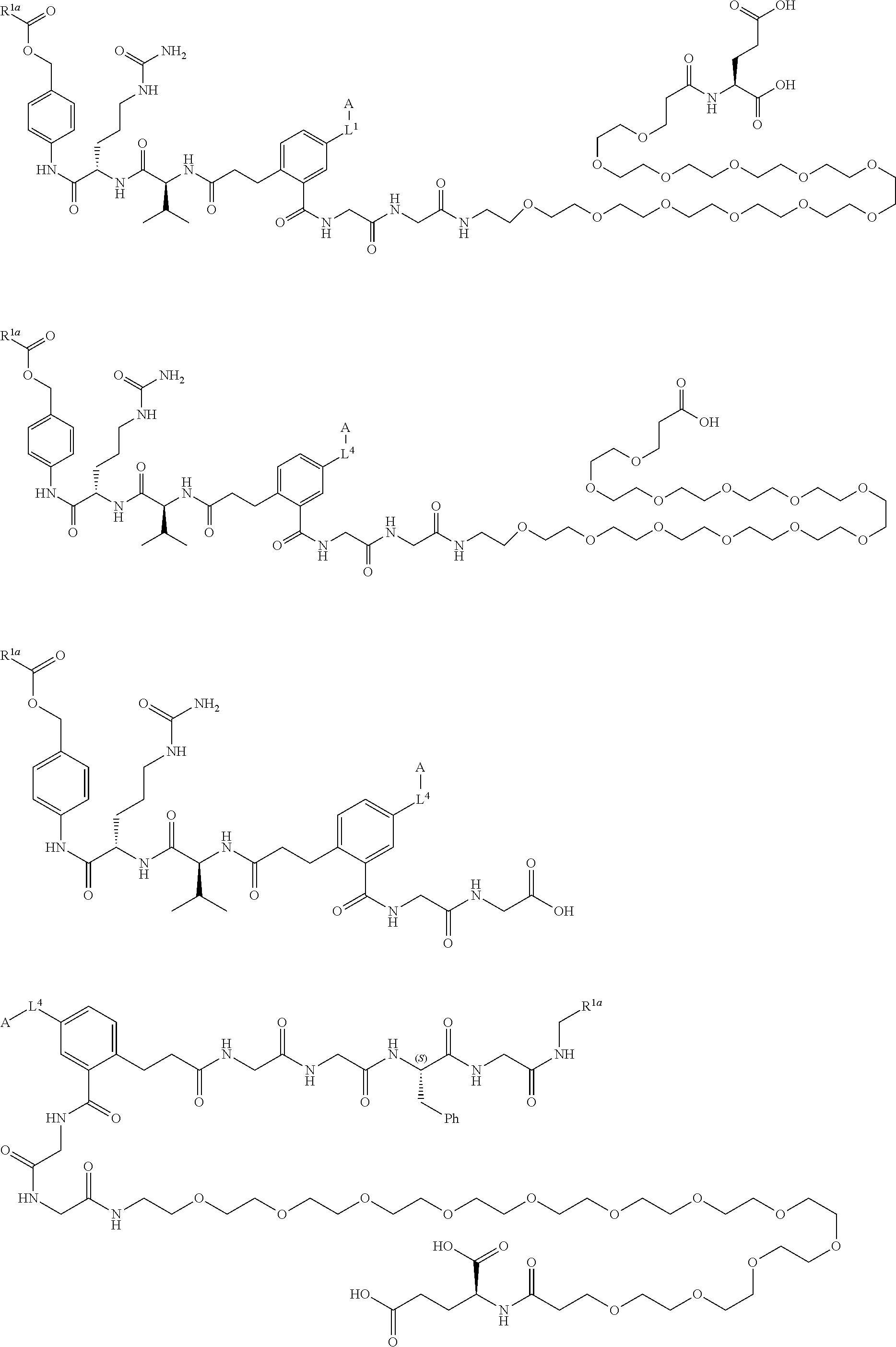

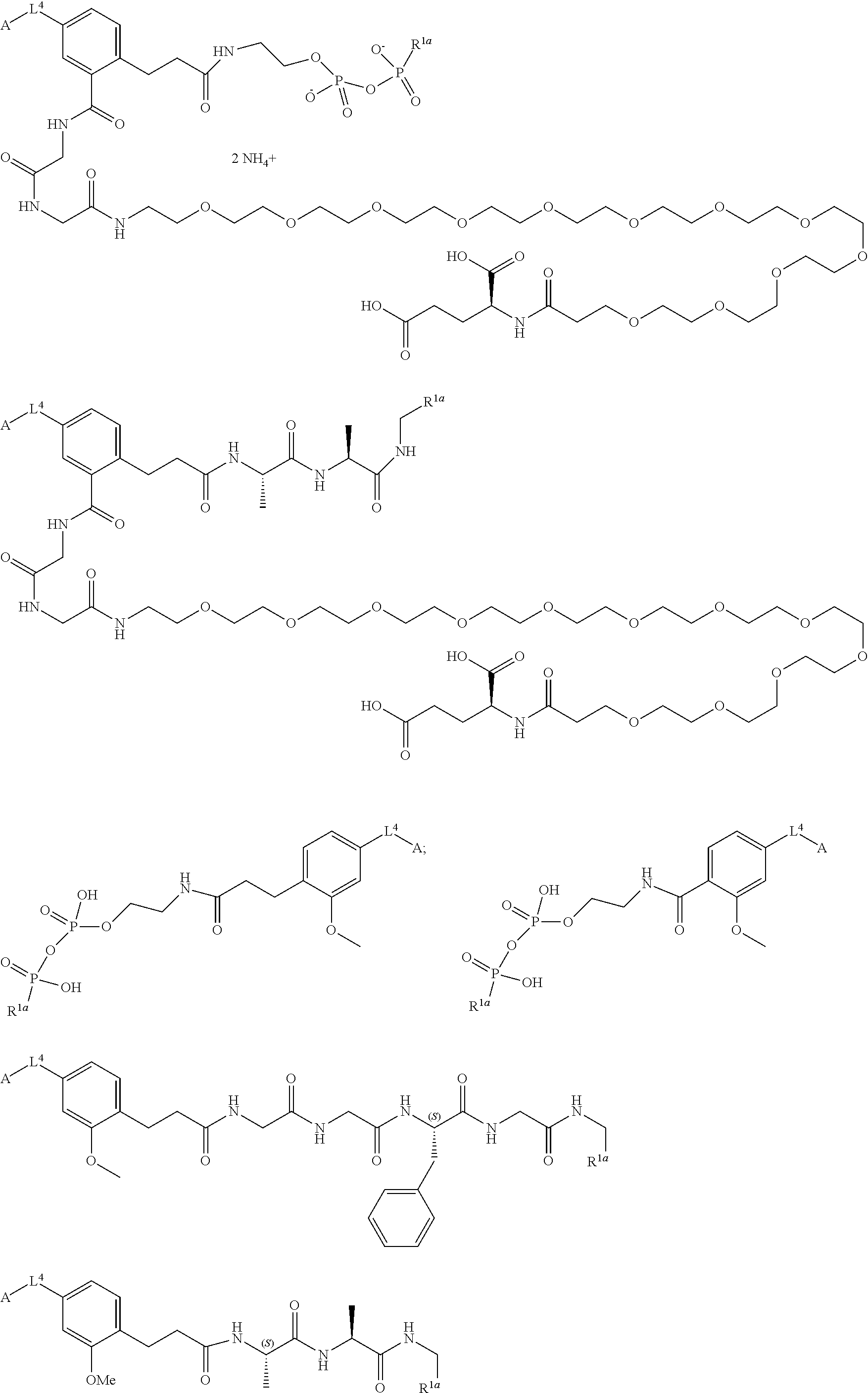

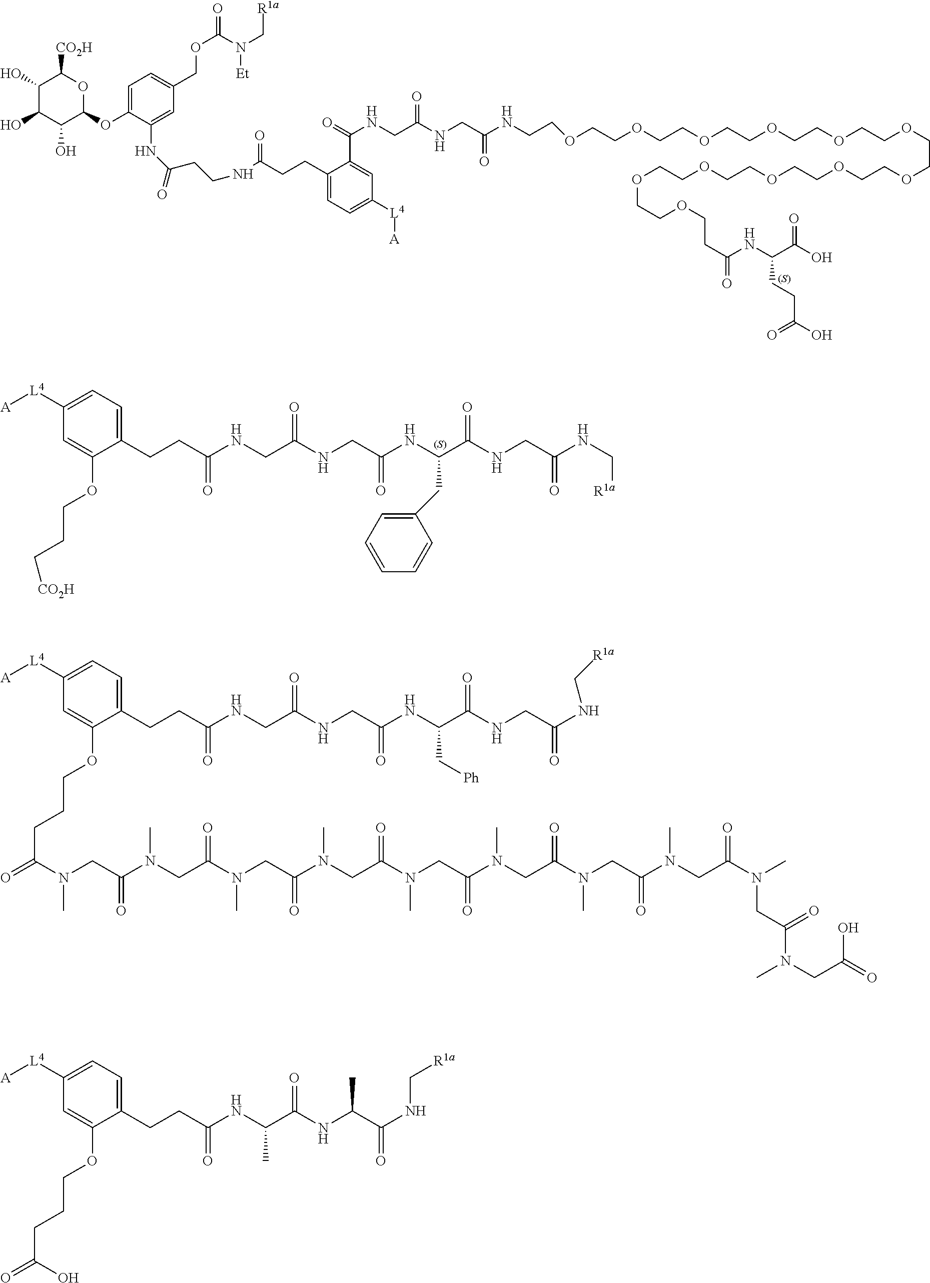

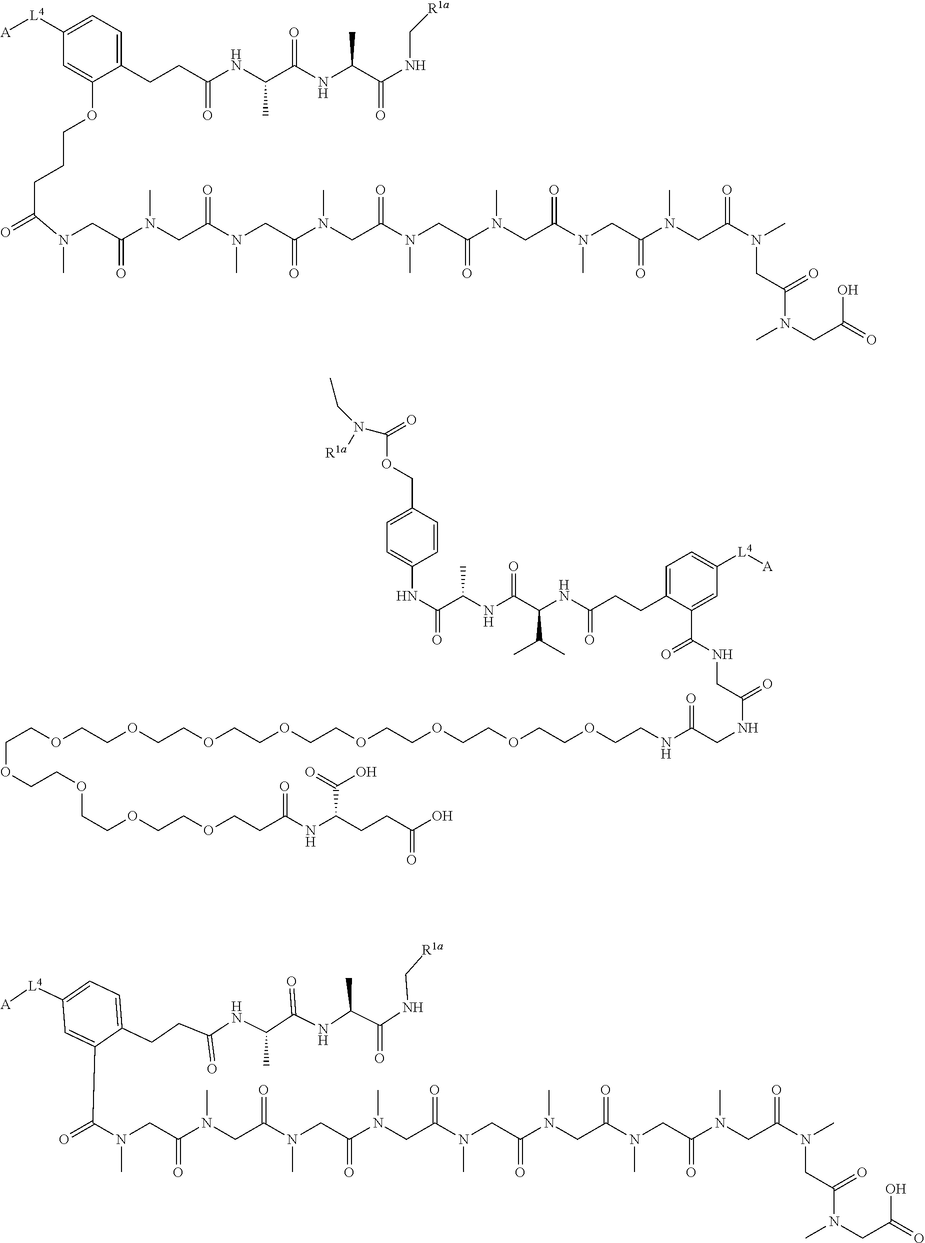

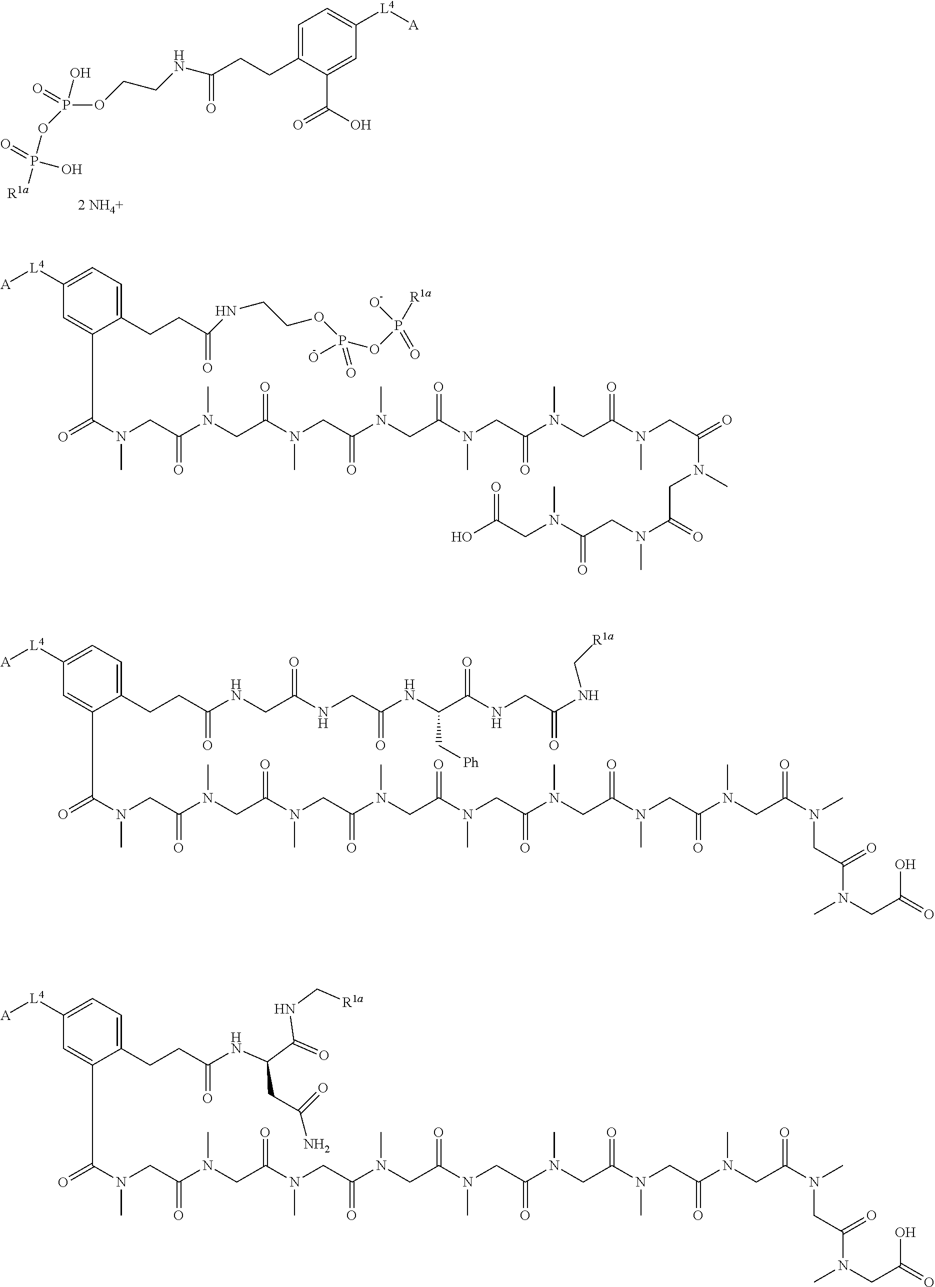

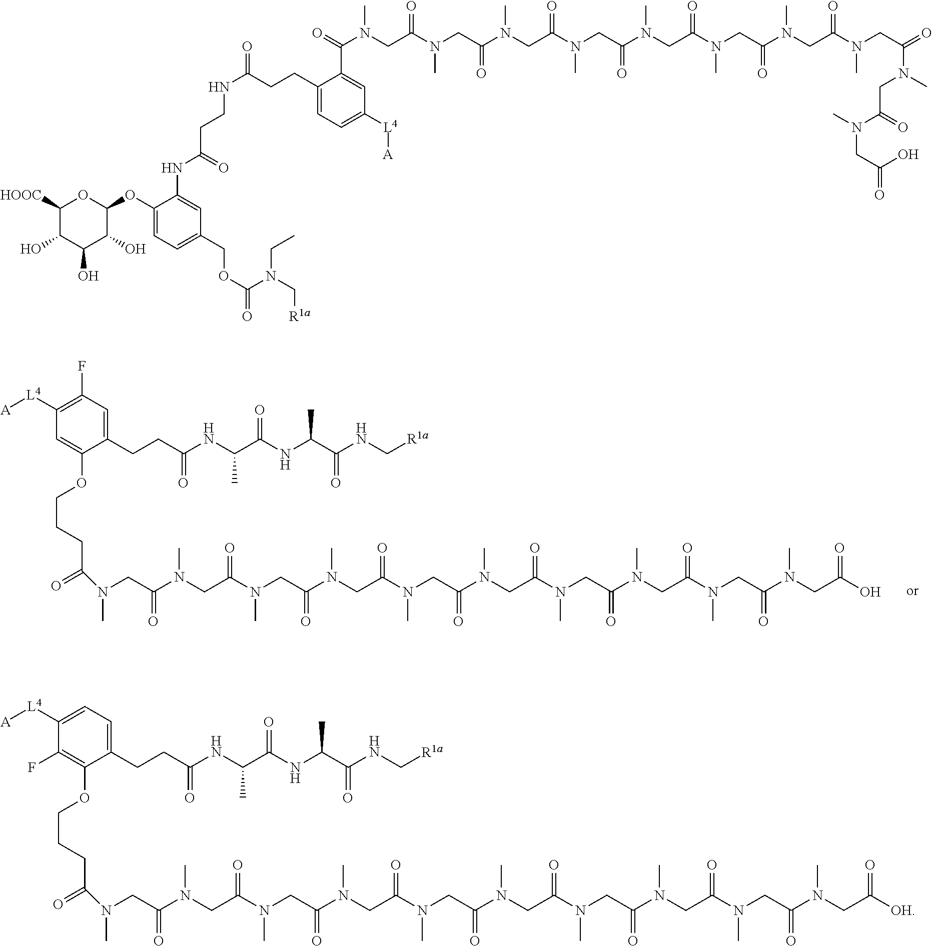

In certain embodiments, R^{1a} has one of the following structures:

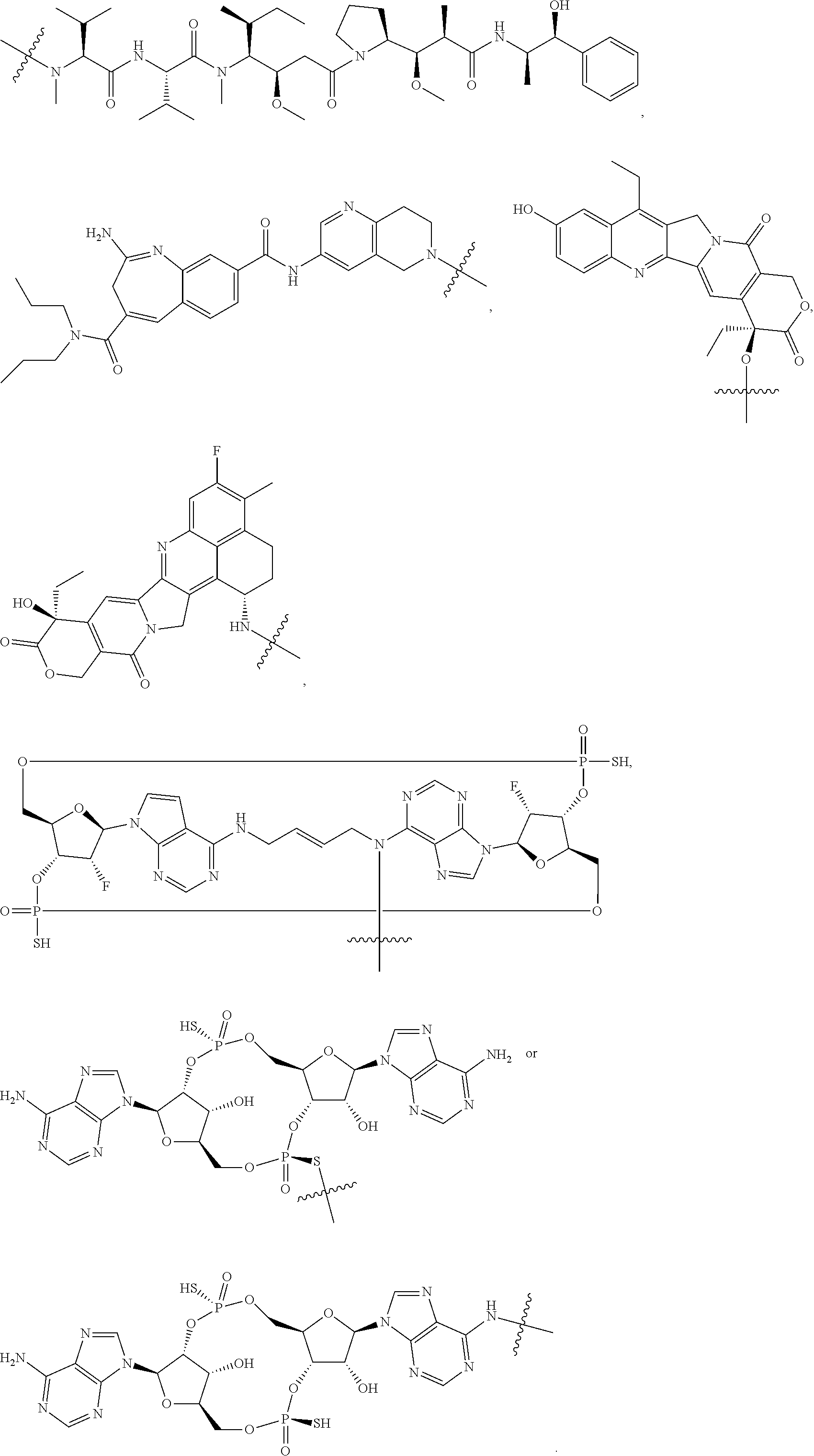

In some embodiments, R^{1a} has one of the following structures:

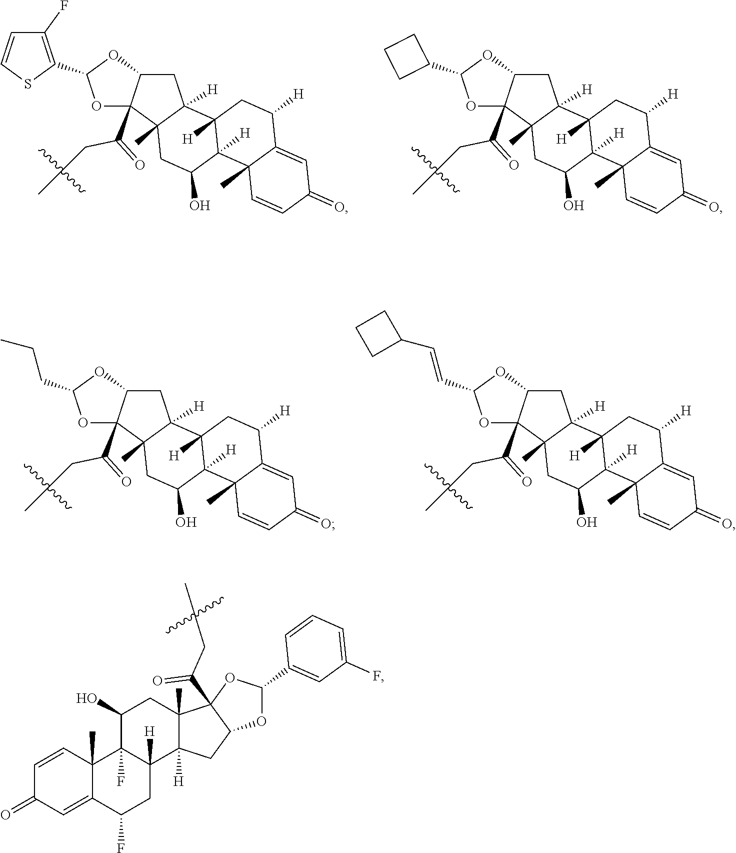

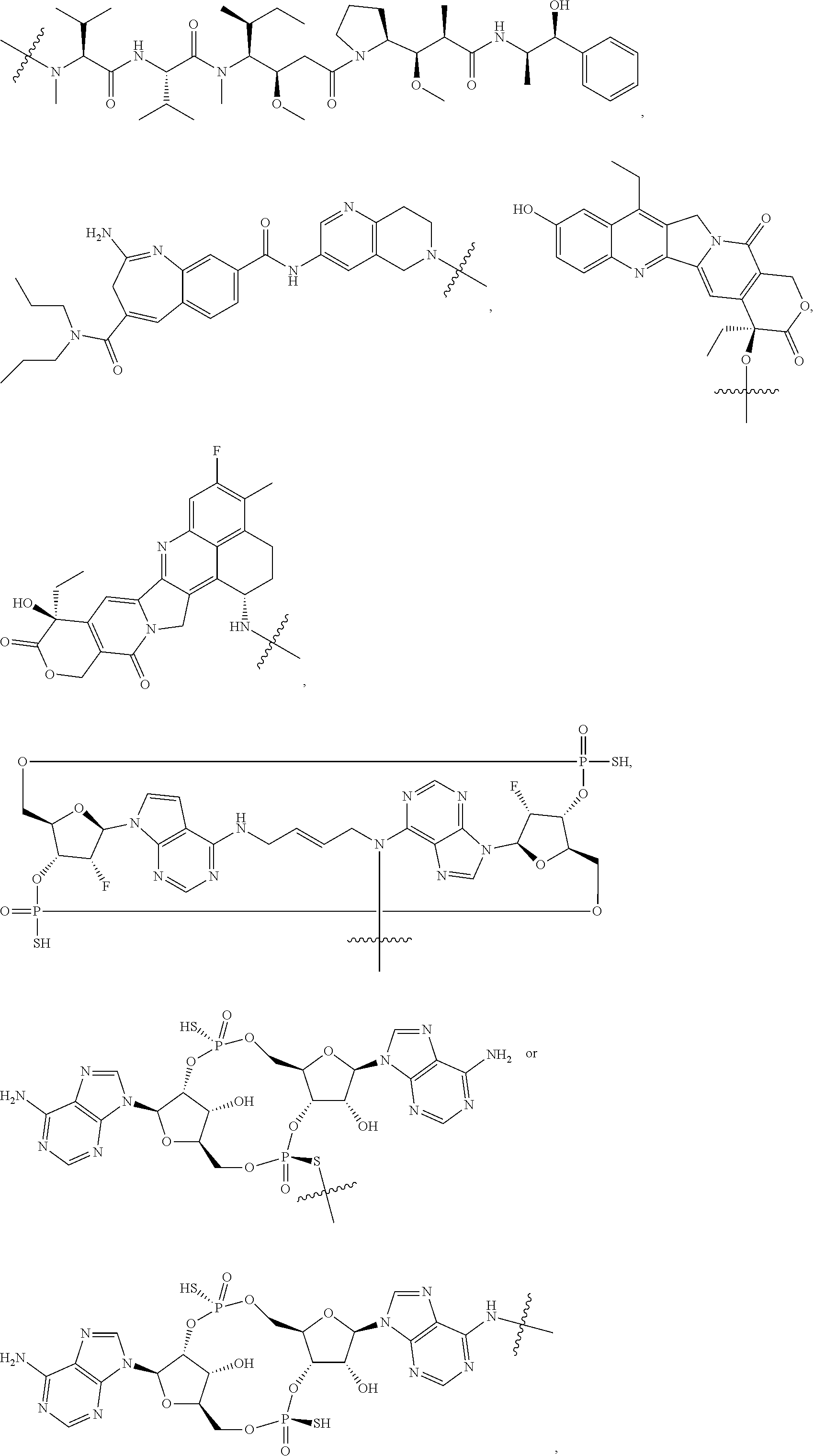

as a stereoisomer, enantiomer or tautomer thereof or a mixture thereof; or a pharmaceutically acceptable salt, solvate or prodrug thereof.

In some embodiments, R^{1a} has one of the following structures:

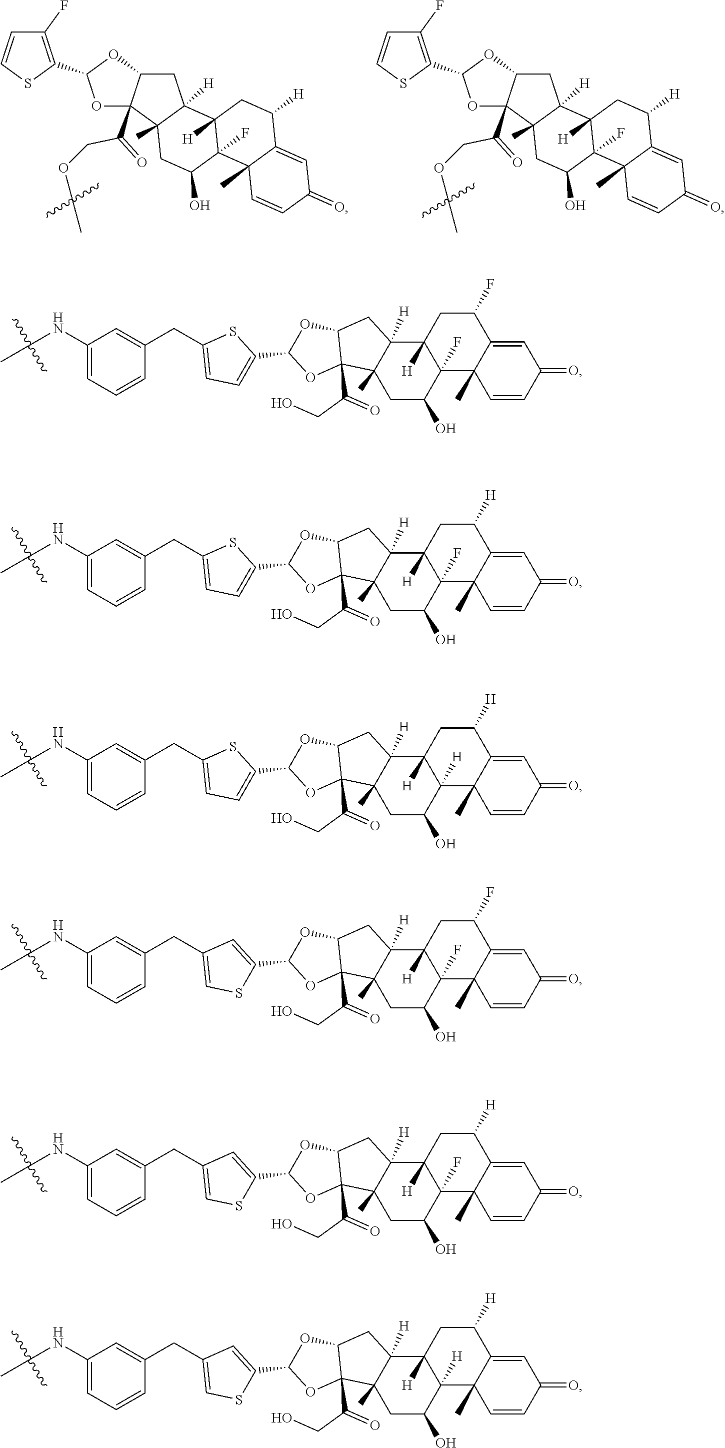

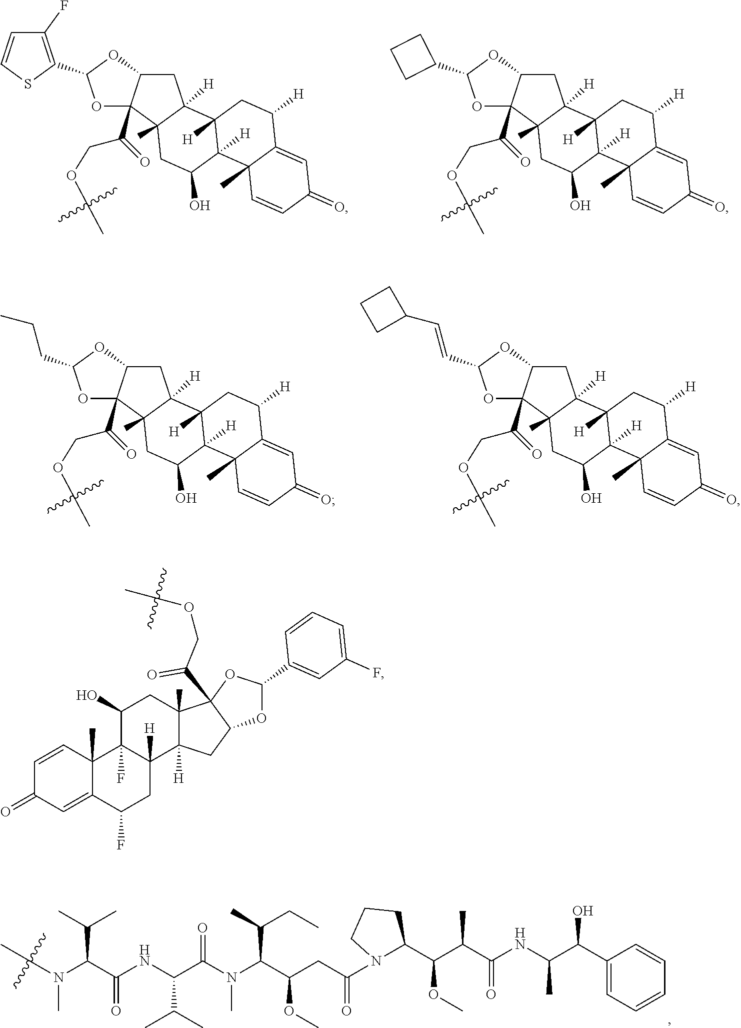

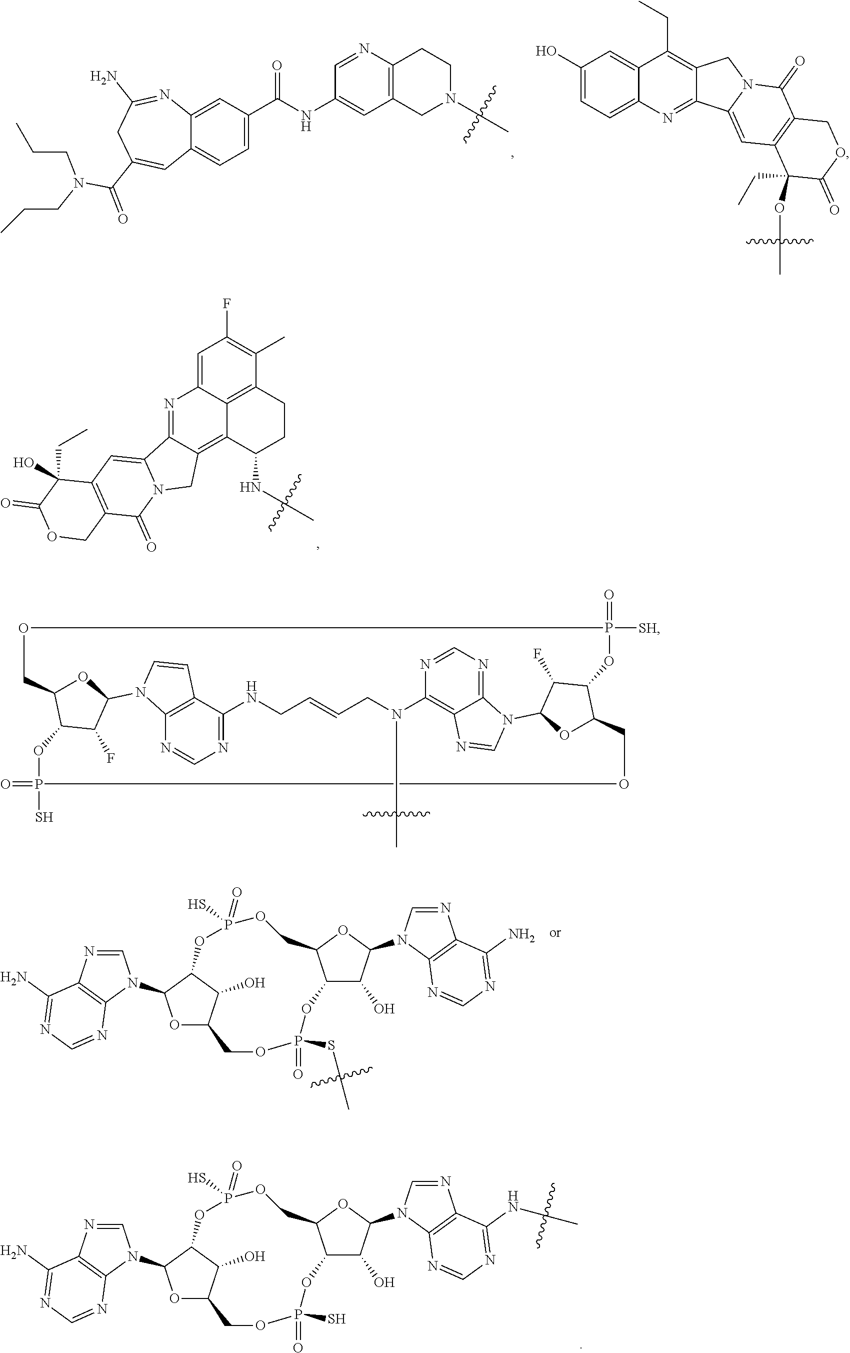

In some embodiments, a payload or R^{1a} has one of the following structures:

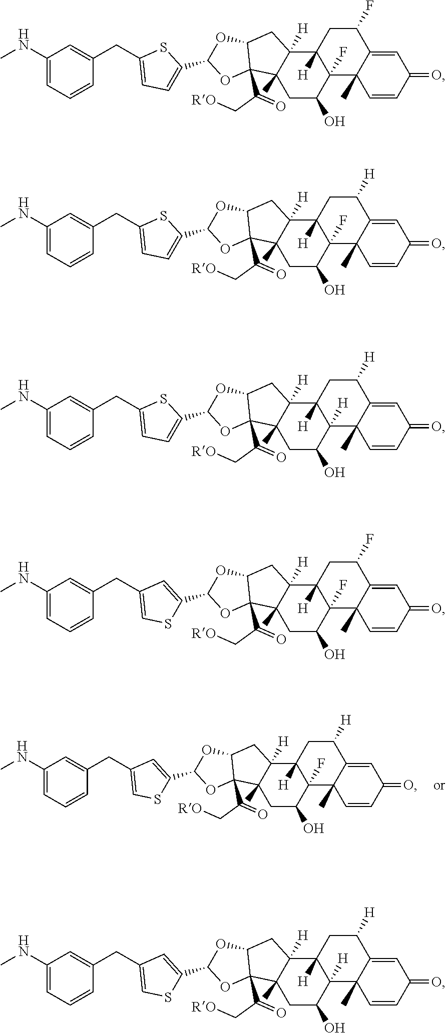

wherein:
R′ is hydrogen or has one of the following structures:

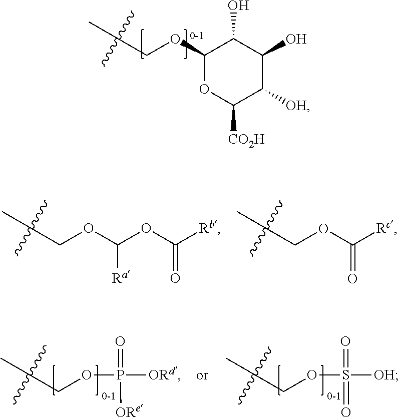

wherein:
R^{a}′ is H or C₁₋₆ alkyl;
R^{b}′ is C₁₋₆ alkyl or C₁₋₆ alkoxy;
R^{c}′ is H, C₁₋₆ alkyl, —CH₂OH, or C₁₋₆ alkoxy;
R^{d}′ is H or C₁₋₆ alkyl; or
R^{e}′ is H or C₁₋₆ alkyl.

In certain embodiments, L¹ or L² has the following structure:

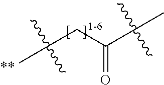

wherein:
** shows a bond to X¹, X², X³, X⁴ or X⁵.

In some embodiments, X² is C-L¹-R¹ and X³ is C-L²-R². In some embodiments, X³ is C-L¹-R¹ and X² is C-L²-R².

In some embodiments, L⁴ has the following structure:

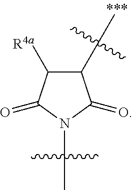

In some embodiments, L⁴ has the following structure:

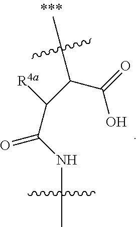

In certain embodiments, L⁴ has the following structure:

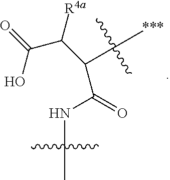

In some embodiments, a targeting moiety is an anti-CD40 antibody, an antibody selected from an anti-LRRC15 antibody, an anti-CTSK antibody, an anti-ADAM12 antibody, an anti-ITGA11 antibody, an anti-FAP antibody, an anti-NOX4 antibody, an anti-SGCD antibody, an anti-SYNDIG1 antibody, an anti-CDH11 antibody, an anti-PLPP4 antibody, an anti-SLC24A2 antibody, an anti-PDGFRB antibody, an anti-THY1 antibody, an anti-ANTXR1 antibody, an anti-GAS1 antibody, an anti-CALHM5 antibody, an anti-SDC1 antibody, an anti-HER2 antibody, an anti-TROP2 antibody, an anti-MSLN antibody, an anti-Nectin4 antibody, an anti-ASGR1 antibody, and an anti-MUC16 antibody.

In some embodiments, the antibody is a full-length antibody. In certain embodiments, the antibody is an antigen binding fragment. In some embodiments, the antibody is a humanized antibody. In some embodiments, g is 1, 2, 3, 4, 5, 6, 7, 8, 9 or 10.

Certain embodiments provide a conjugate having one of the structures in Table 2, wherein A is a targeting moiety or binding fragment thereof as a stereoisomer, enantiomer or tautomer thereof or a mixture thereof; or a pharmaceutically acceptable salt, solvate or prodrug thereof.

TABLE 2
Representative conjugates of Structure (II)

| Compound | |
|---|---|
| No. | Structure |
| II-1 | 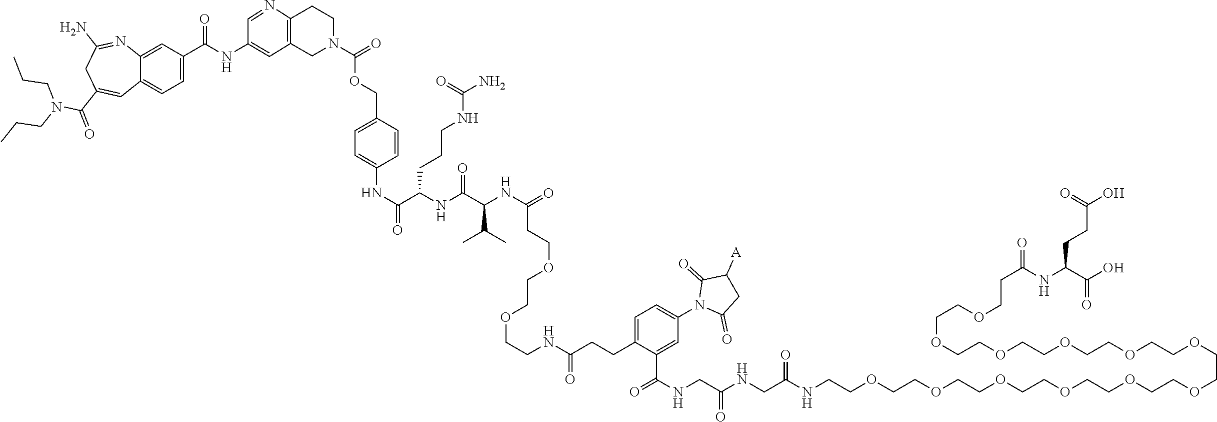 |
| II-2 | 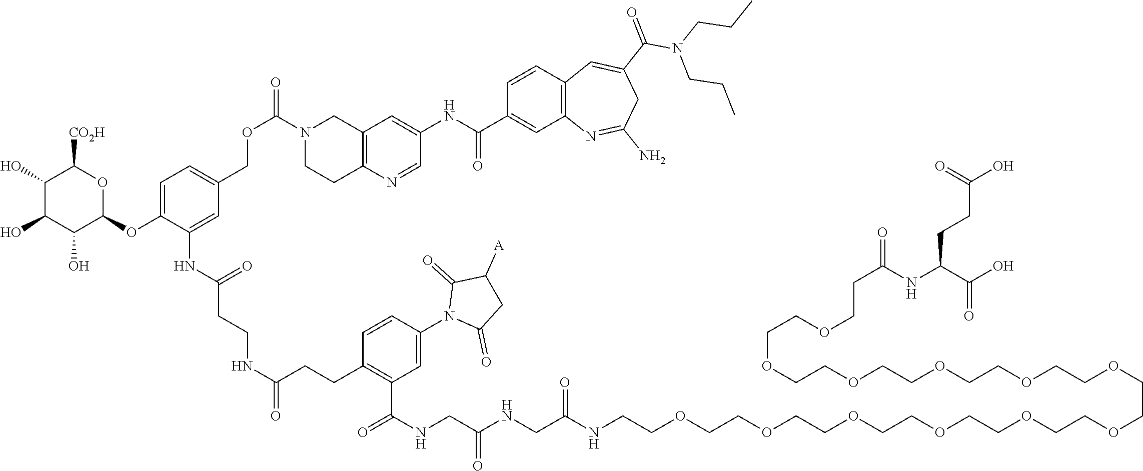 |
| II-3 | 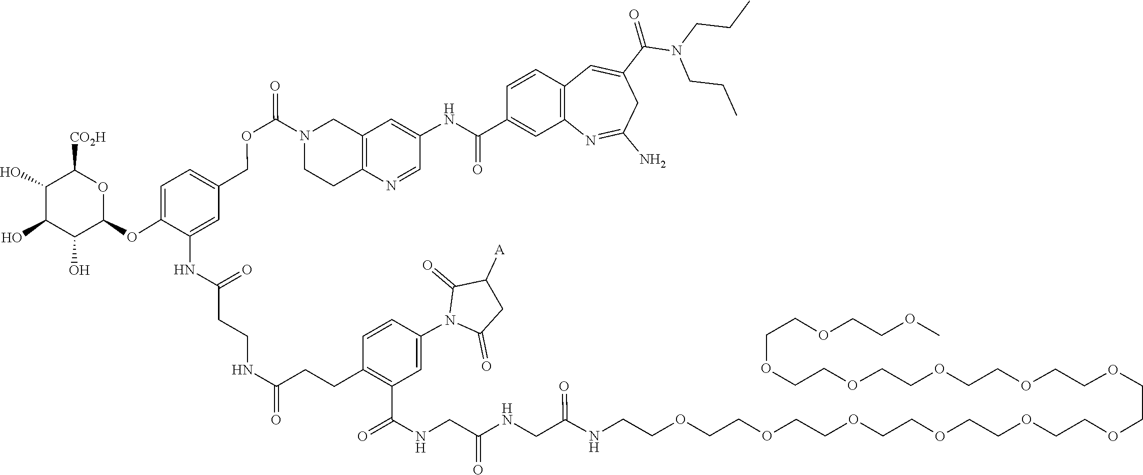 |
| II-4 | 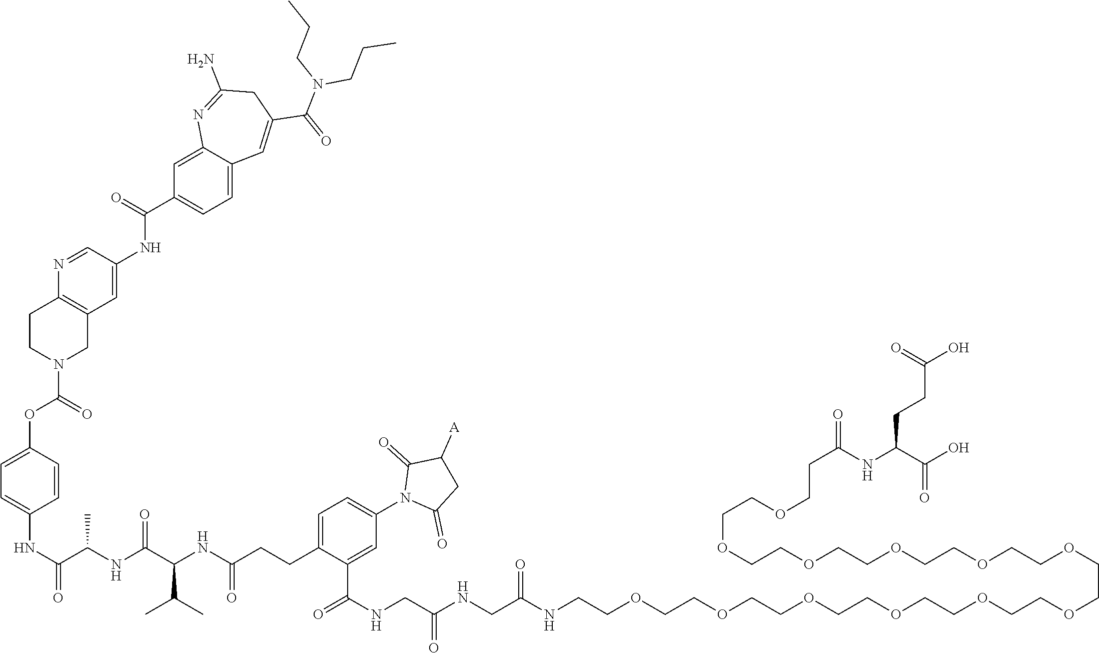 |
| II-5 | 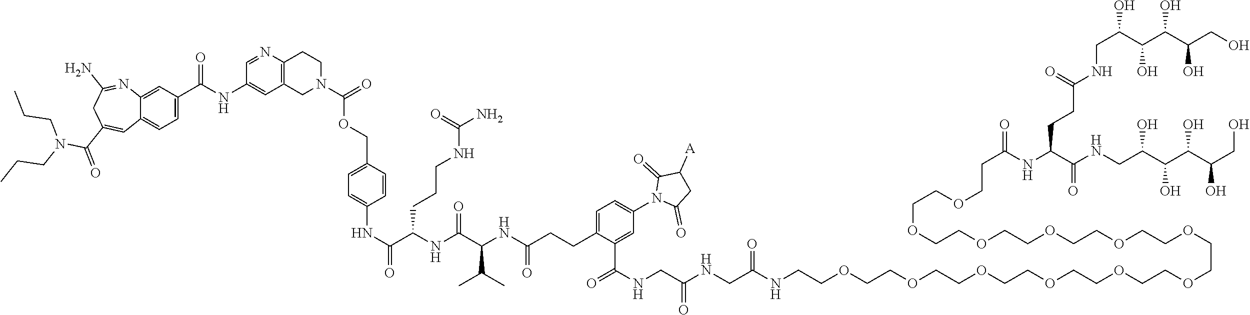 |
| II-6 | 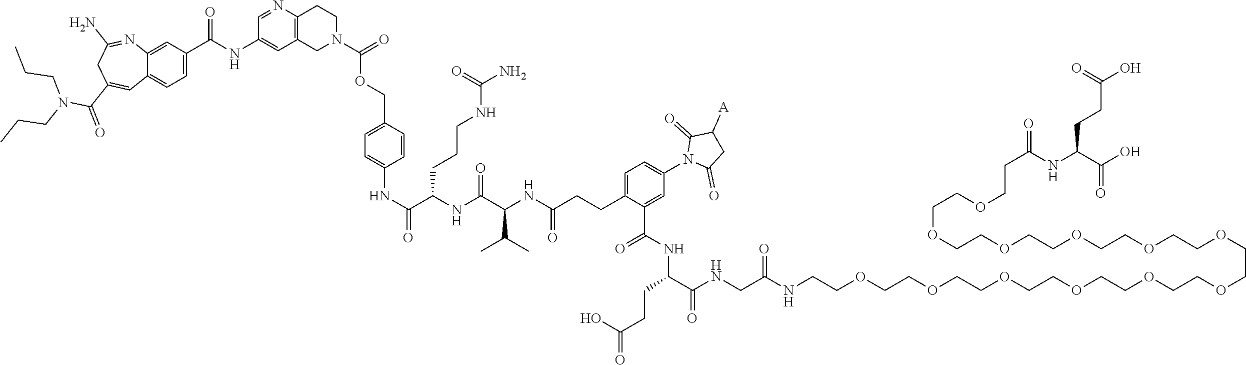 |
| II-7 | 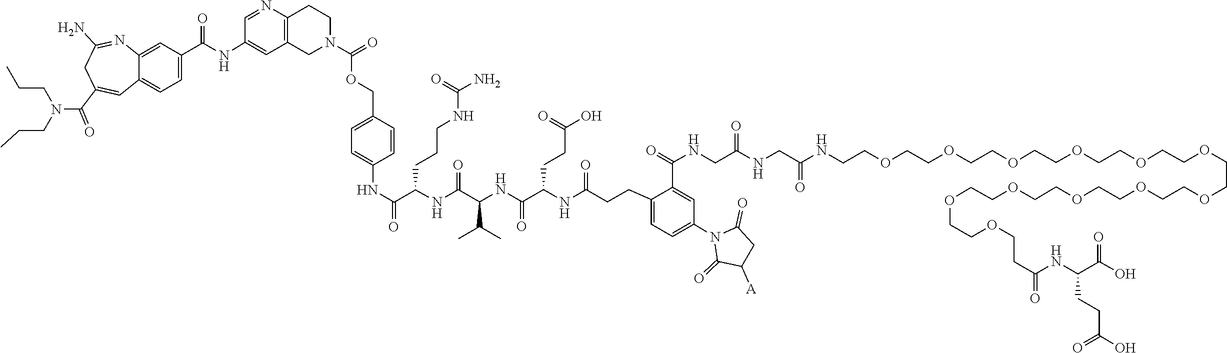 |
| II-8 | 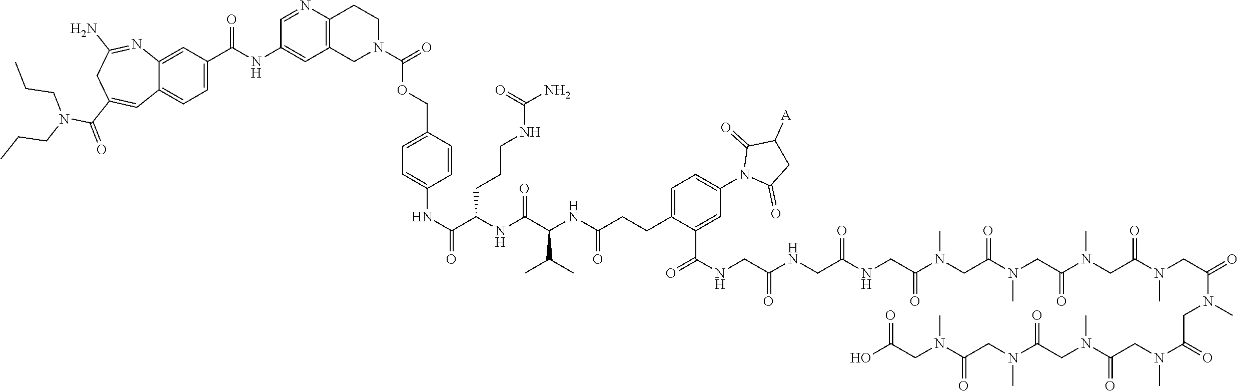 |
| II-9 | 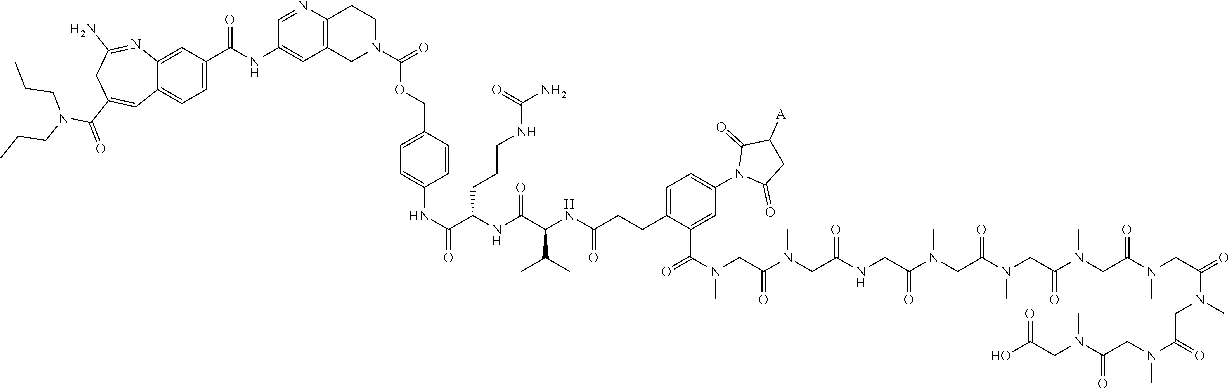 |
| II-10 | 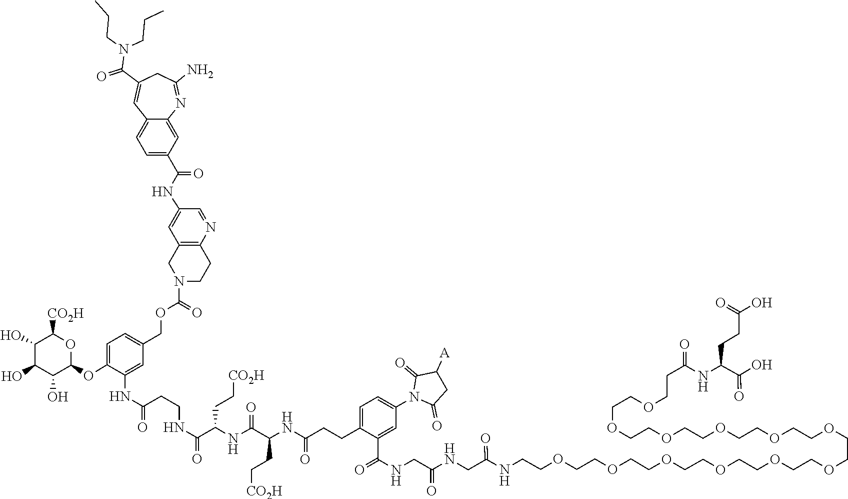 |
| II-11 | 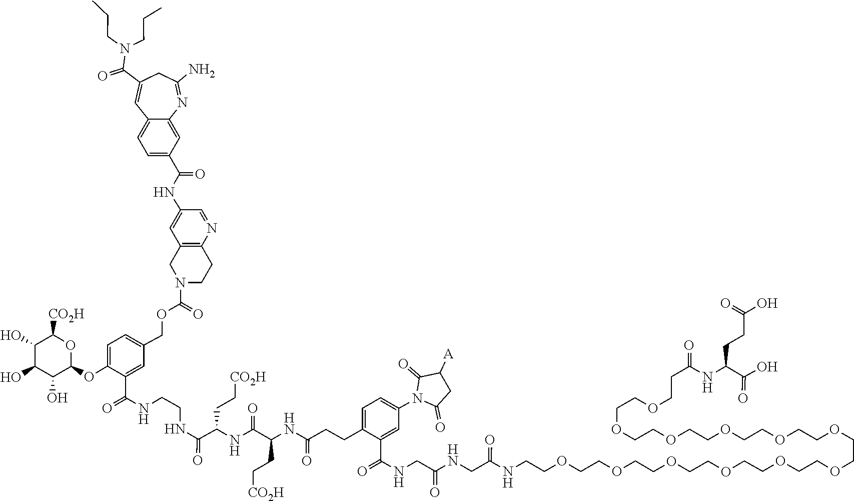 |
| II-12 | 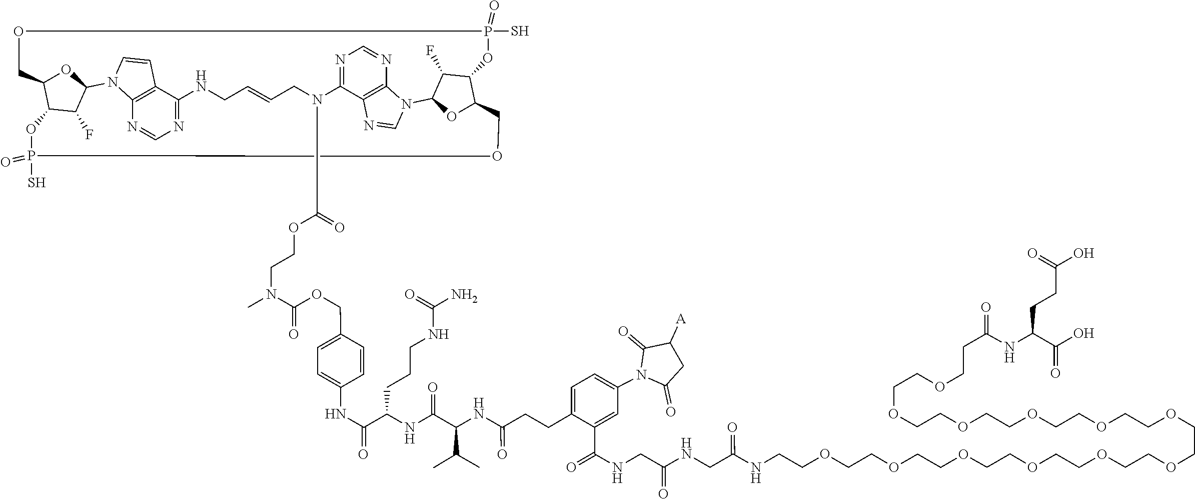 |
| II-13 | 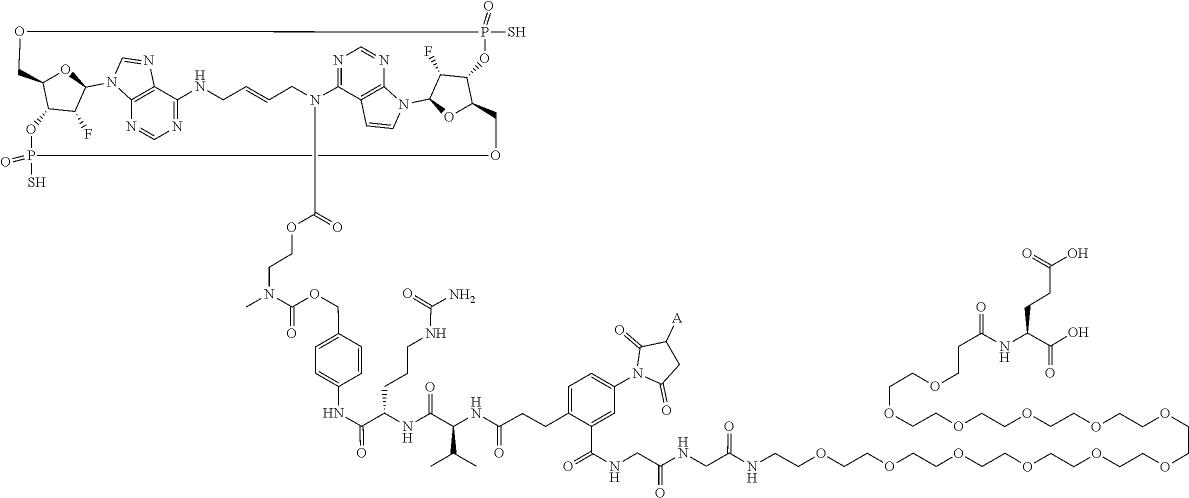 |
| II-14 | 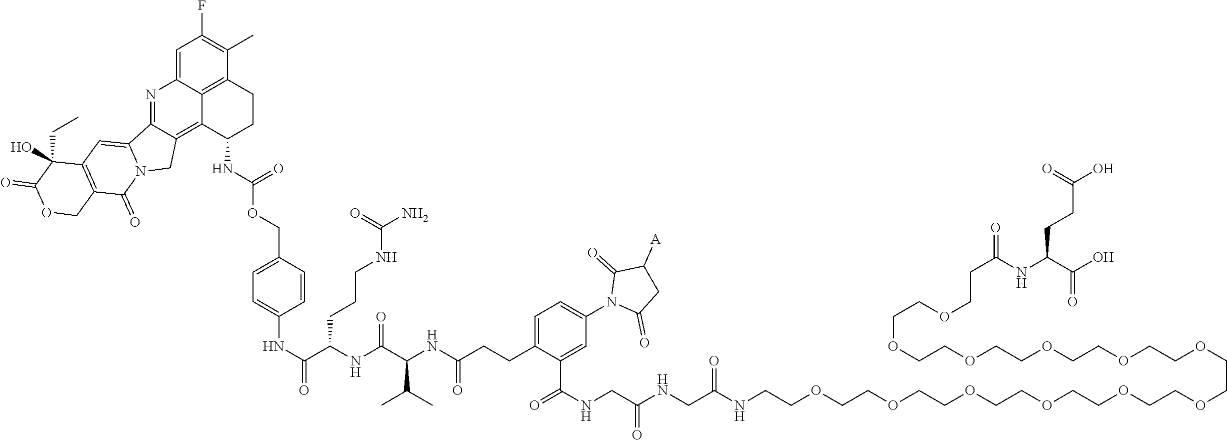 |
| II-15 | 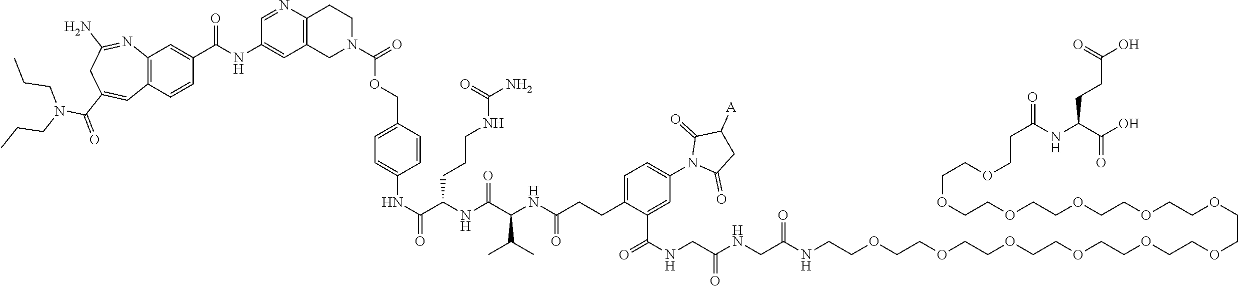 |
| II-16 | 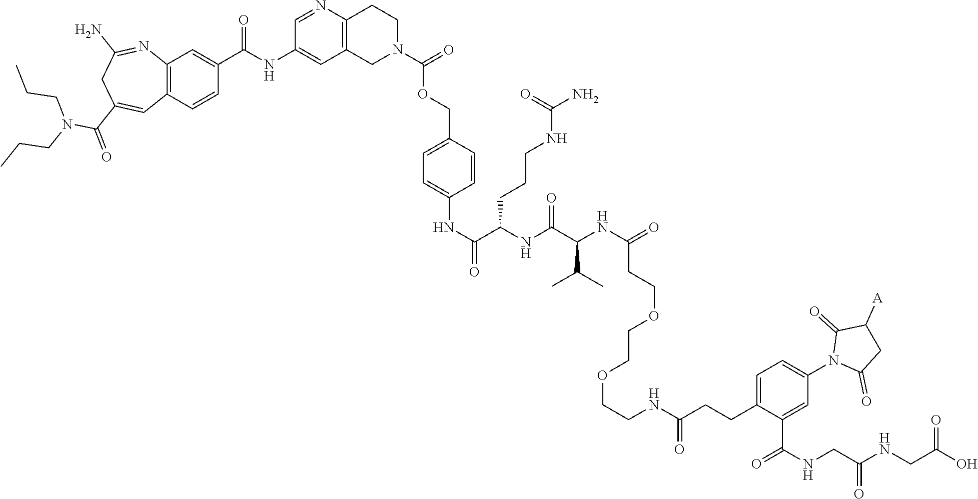 |
| II-17 | 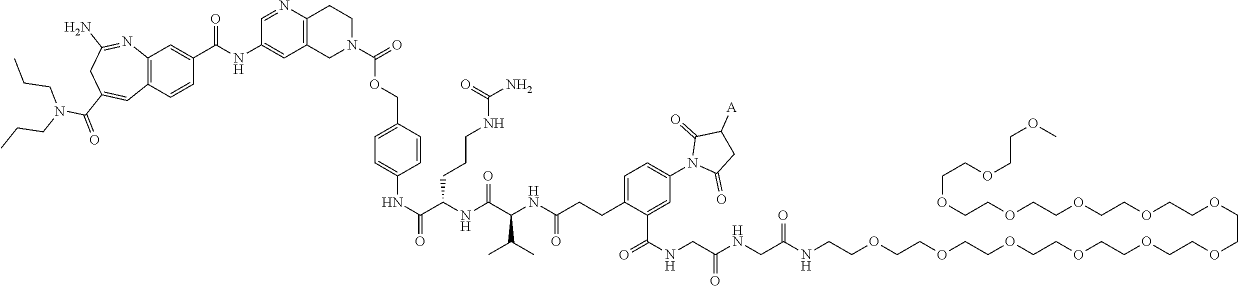 |
| II-18 | 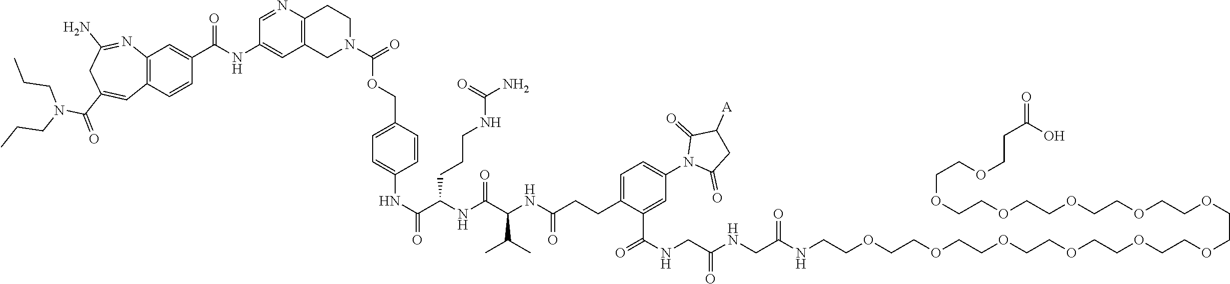 |
| II-19 | 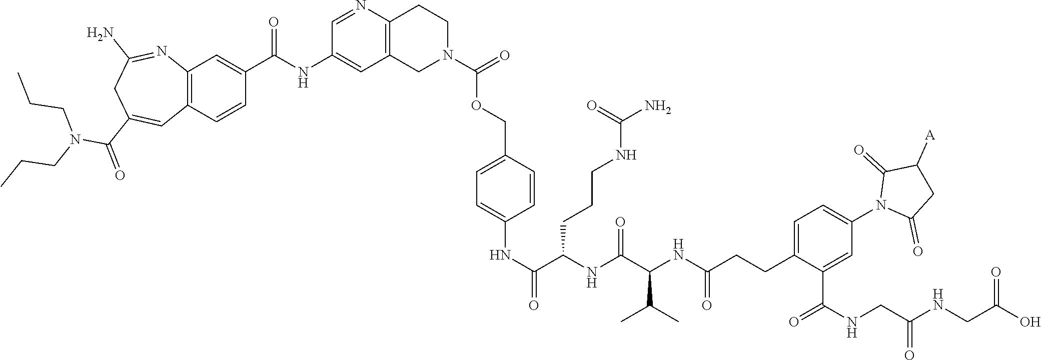 |
| II-20 | 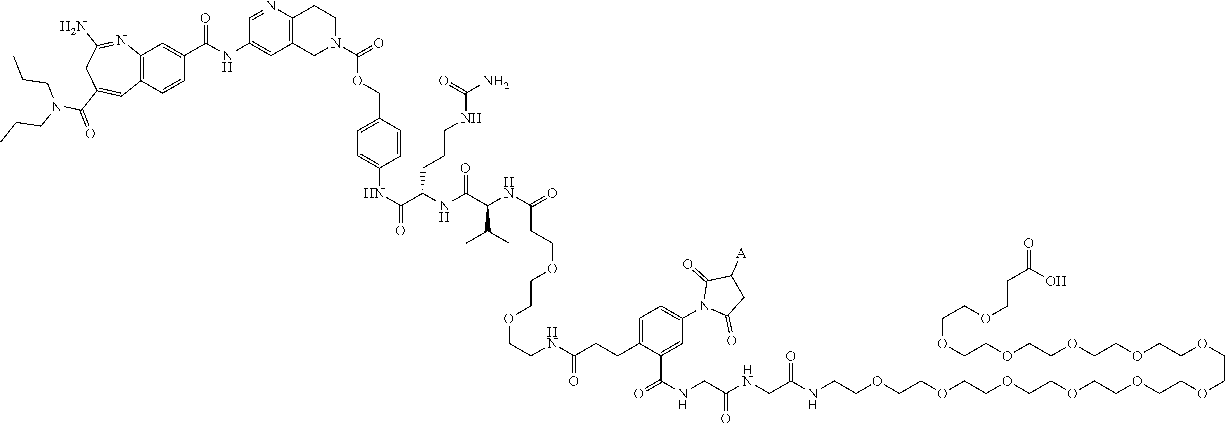 |
| II-21 | 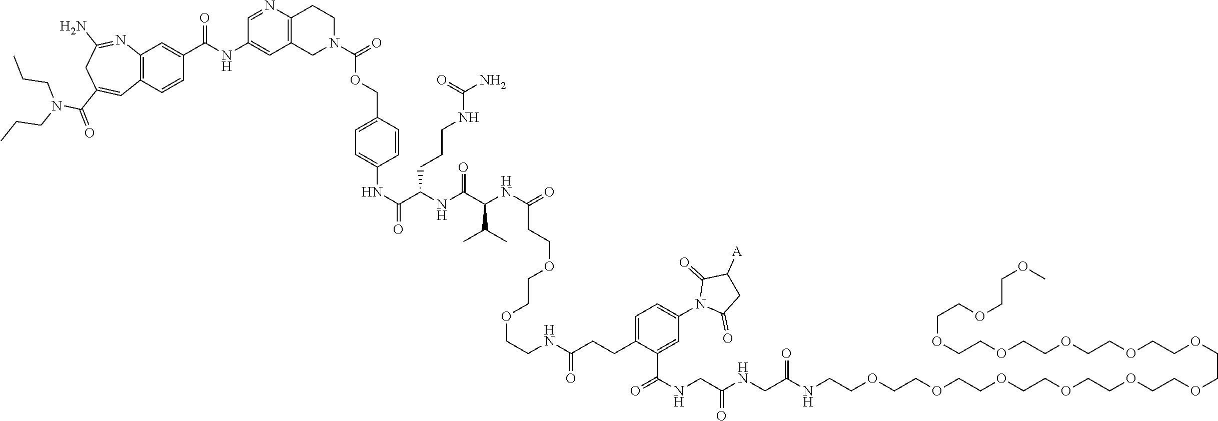 |
| II-22 | 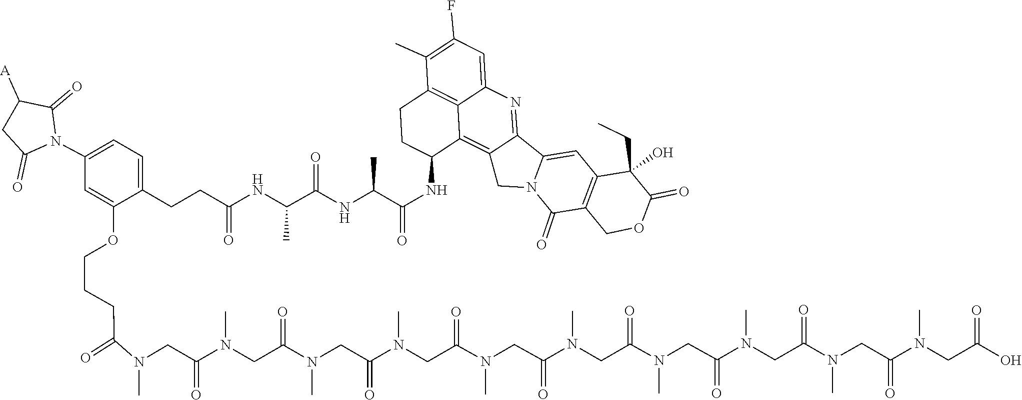 |
| II-23 | 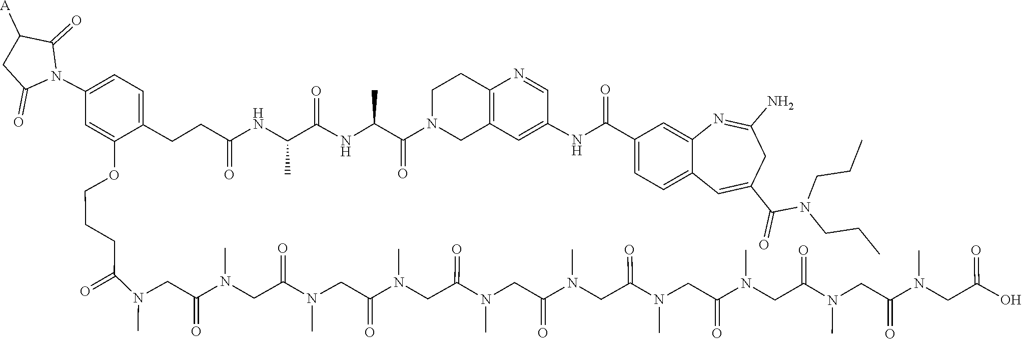 |
| II-24 | 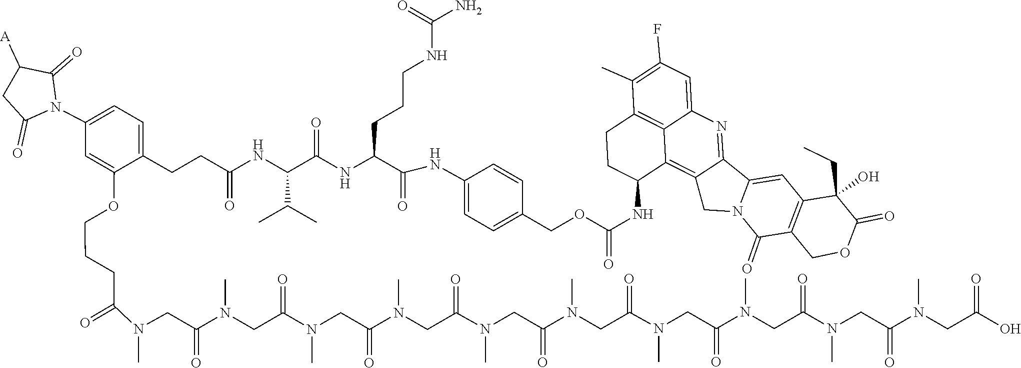 |
| II-25 | 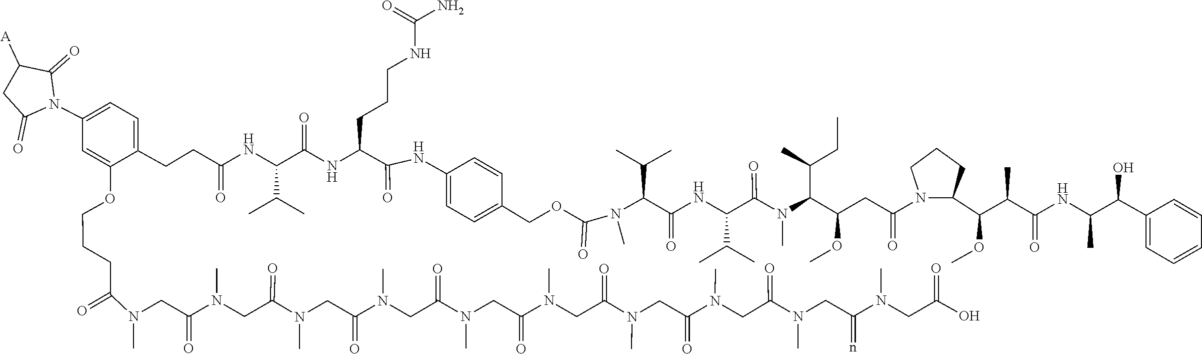 |
| II-26 | 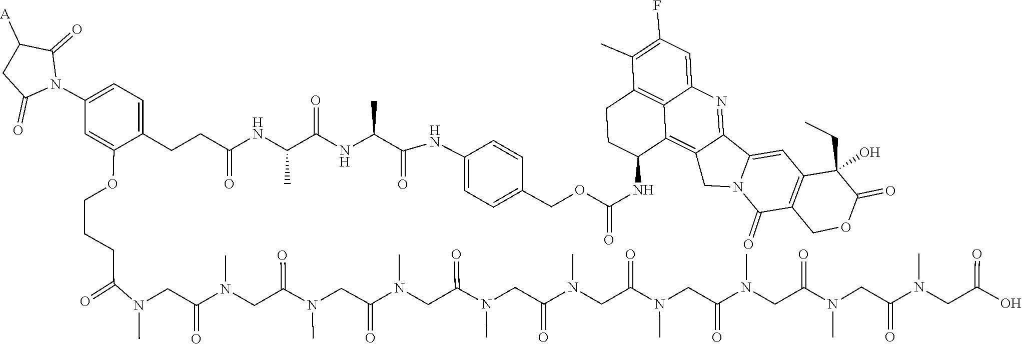 |
| II-27 | 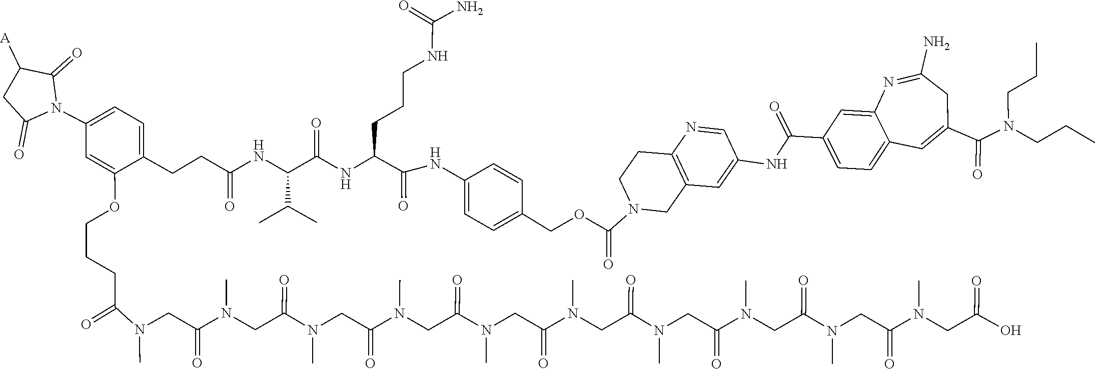 |
| II-28 | 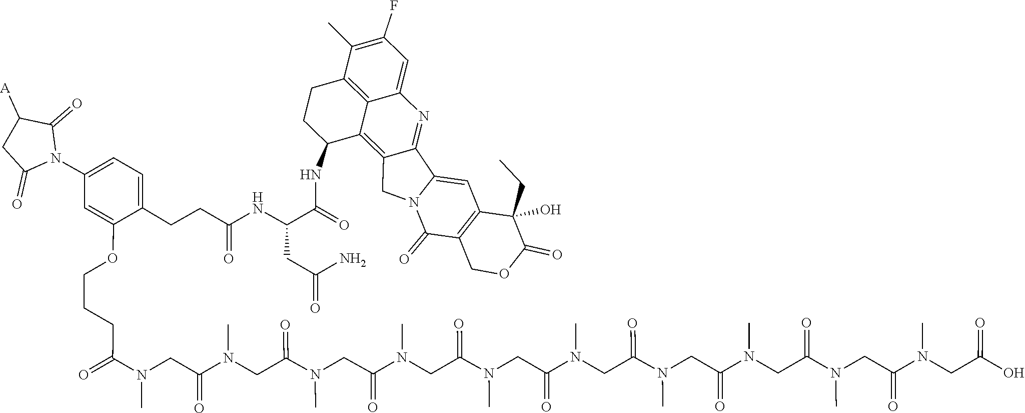 |
| II-29 | 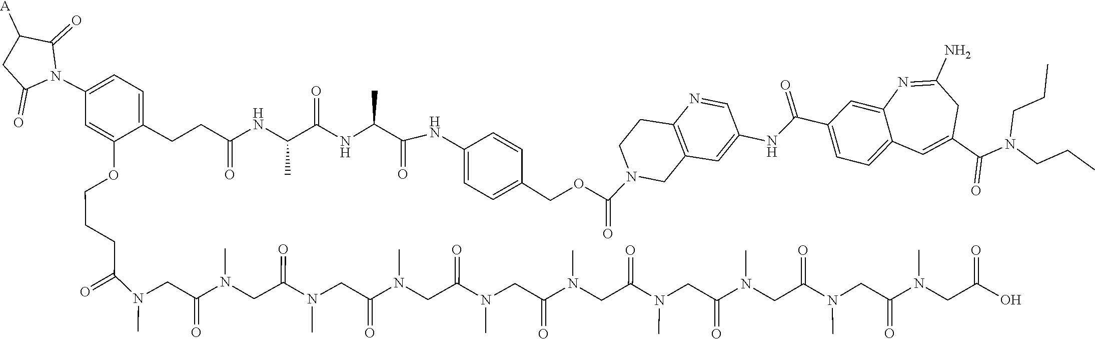 |
| II-30 | 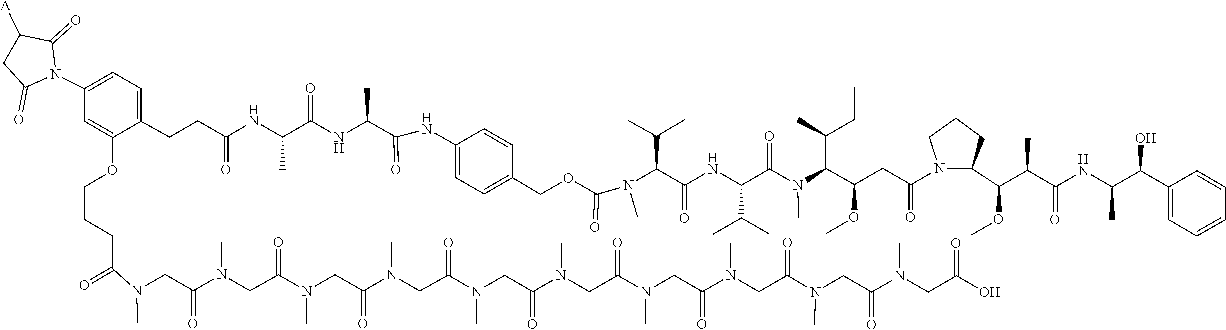 |
| II-31 | 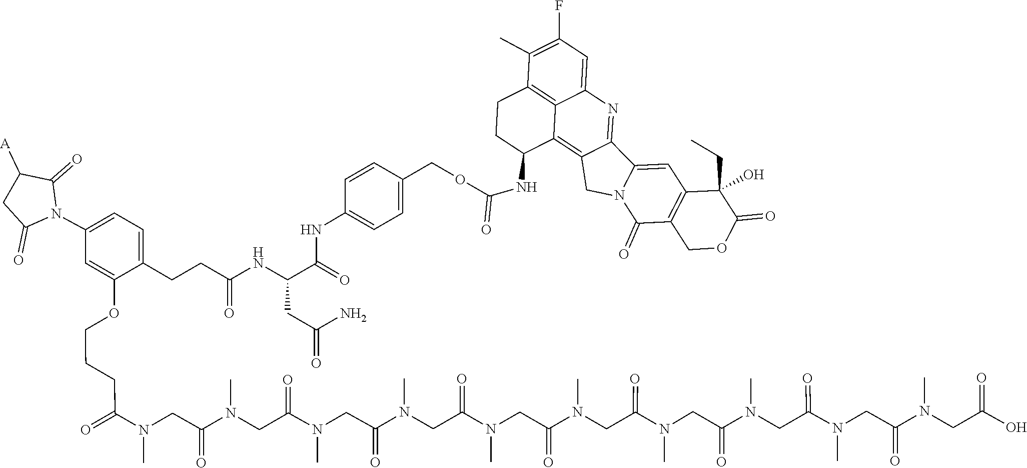 |
| II-32 | 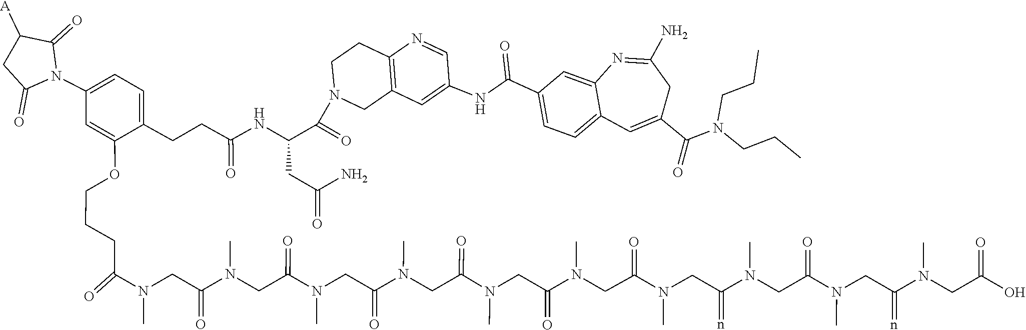 |
| II-33 | 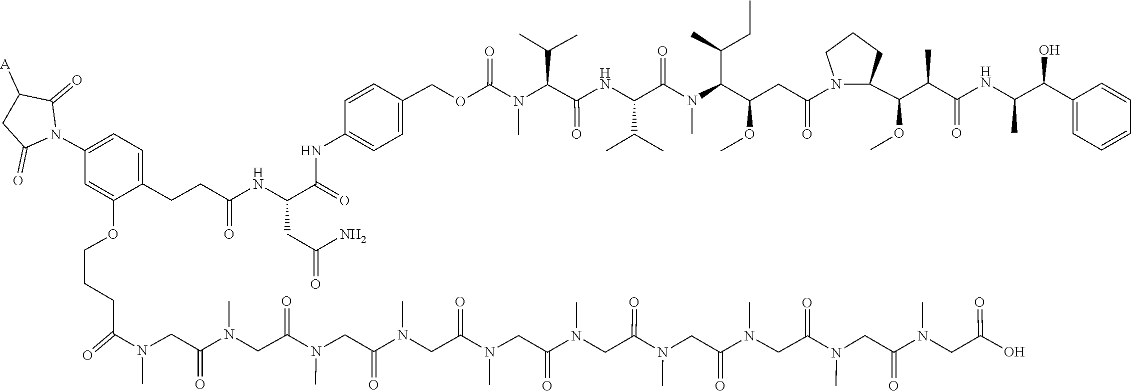 |
| II-34 | 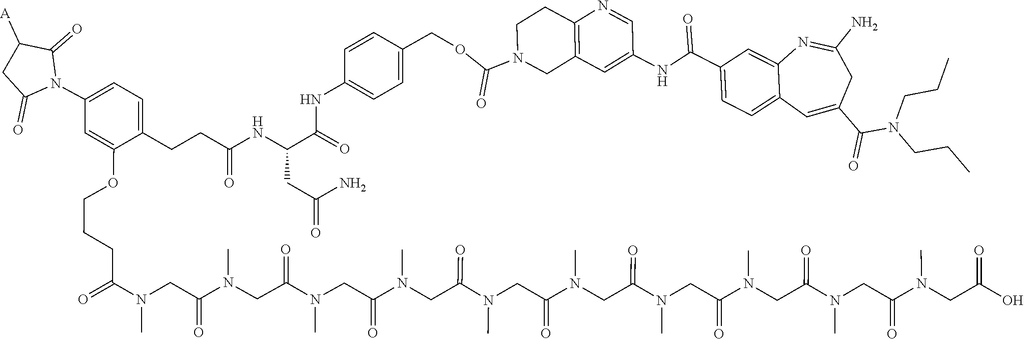 |
| II-35 | 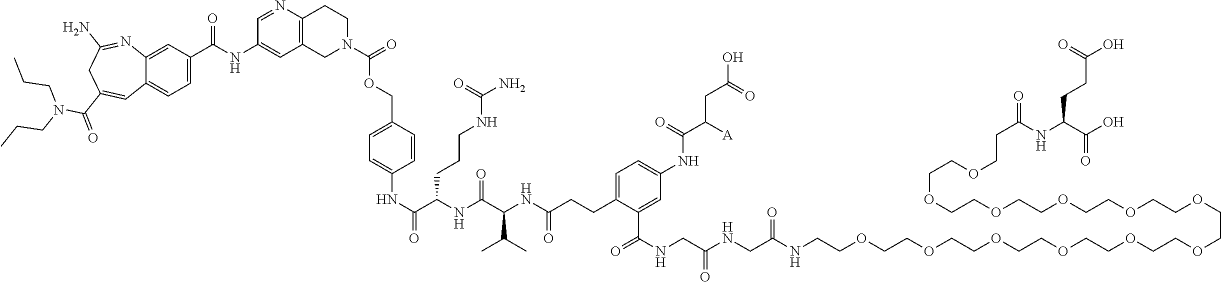 |
| II-36 | 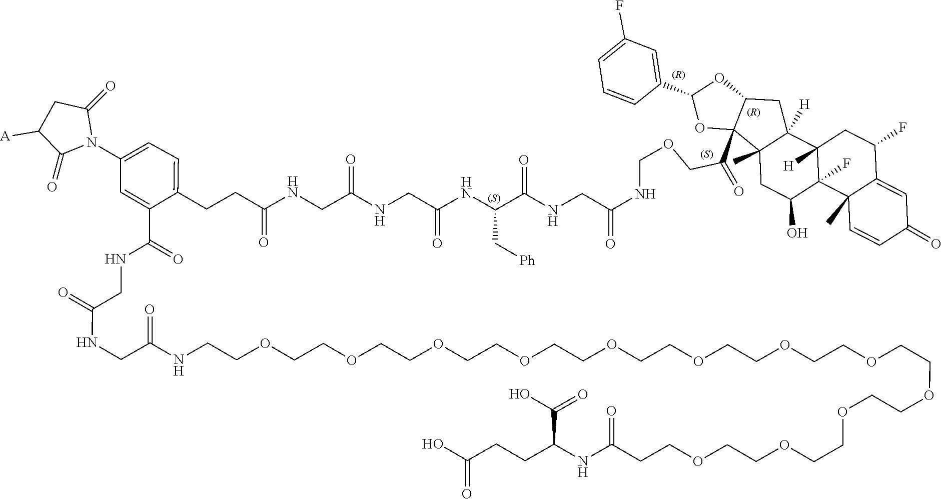 |
| II-37 | 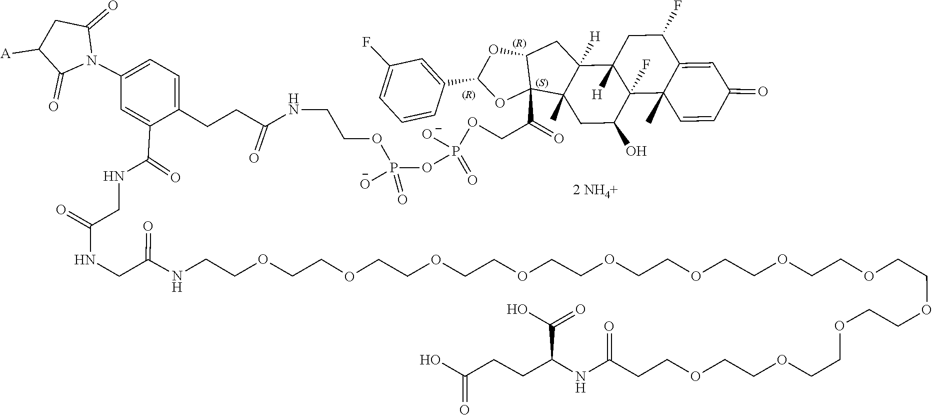 |
| II-38 | 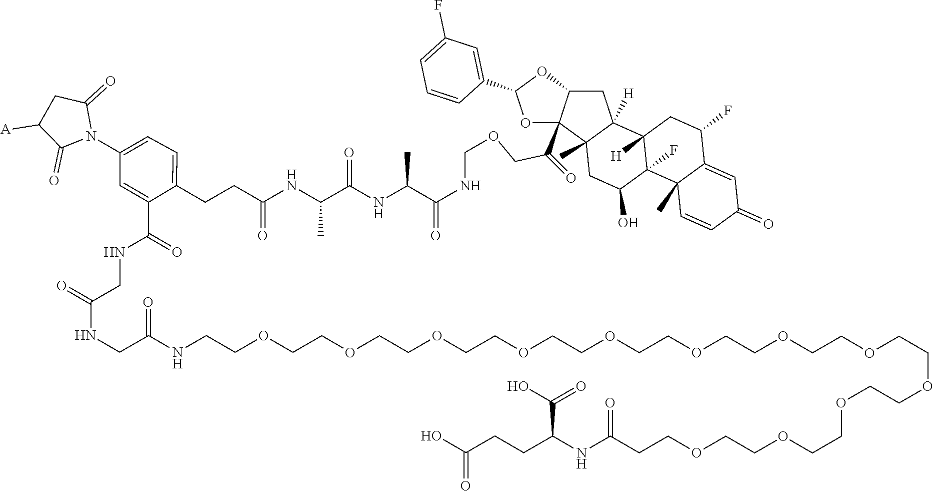 |
| II-39 | 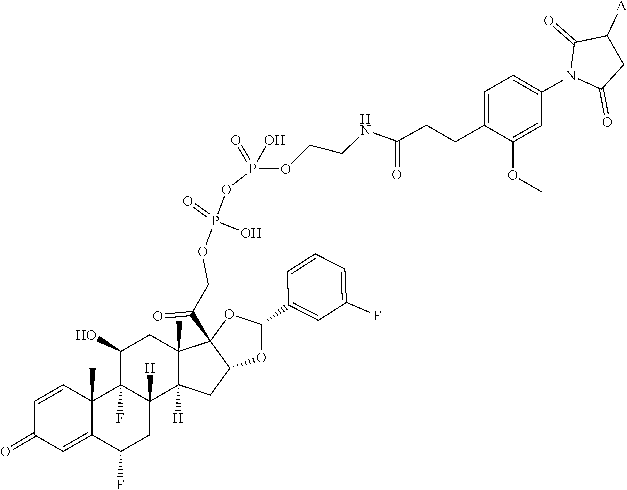 |
| II-40 | 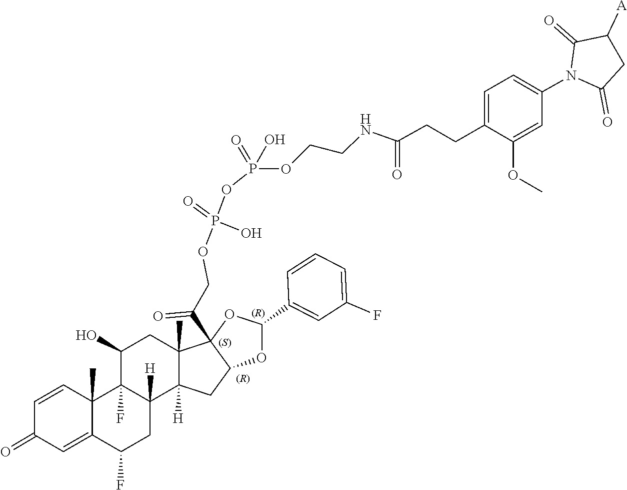 |
| II-41 | 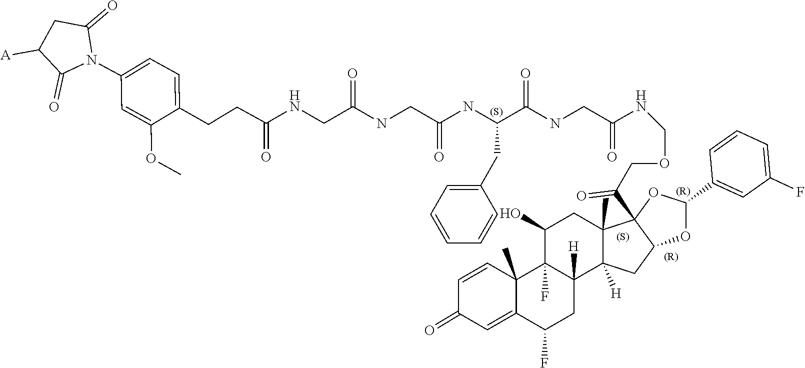 |
| II-42 | 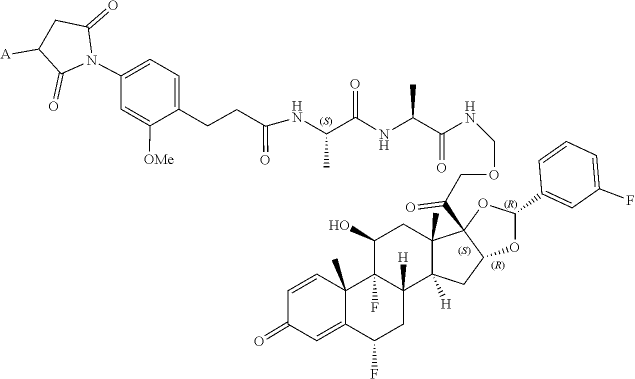 |
| II-43 | 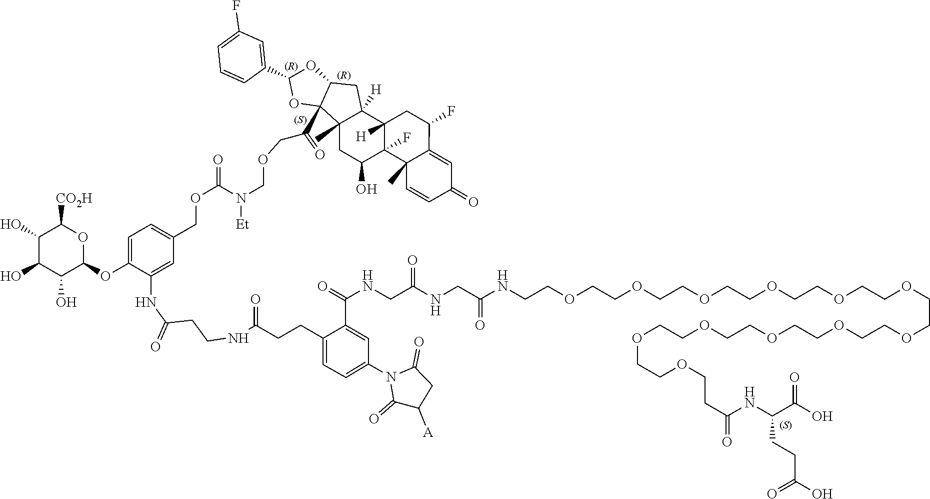 |
| II-44 | 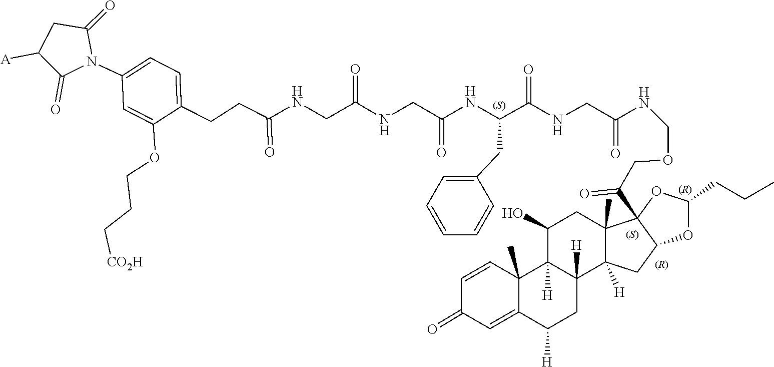 |
| II-45 | 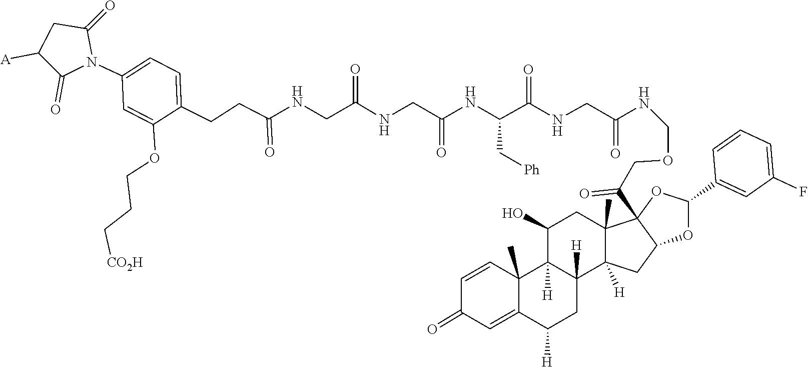 |
| II-46 | 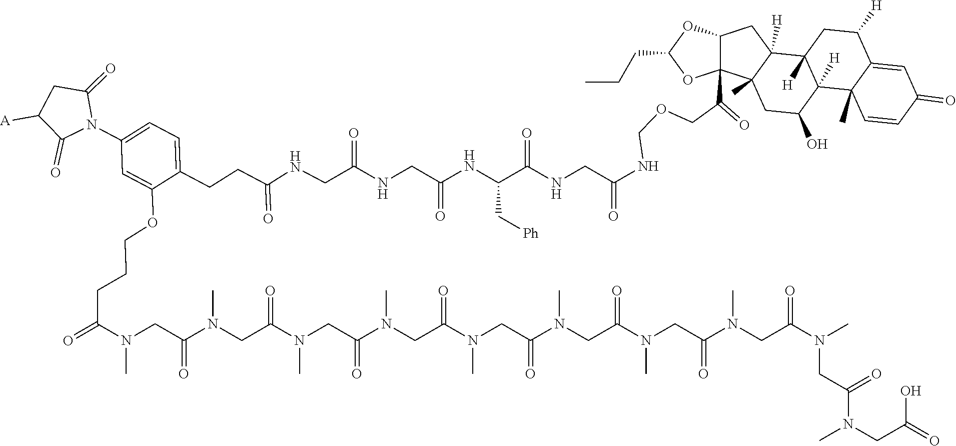 |
| II-47 | 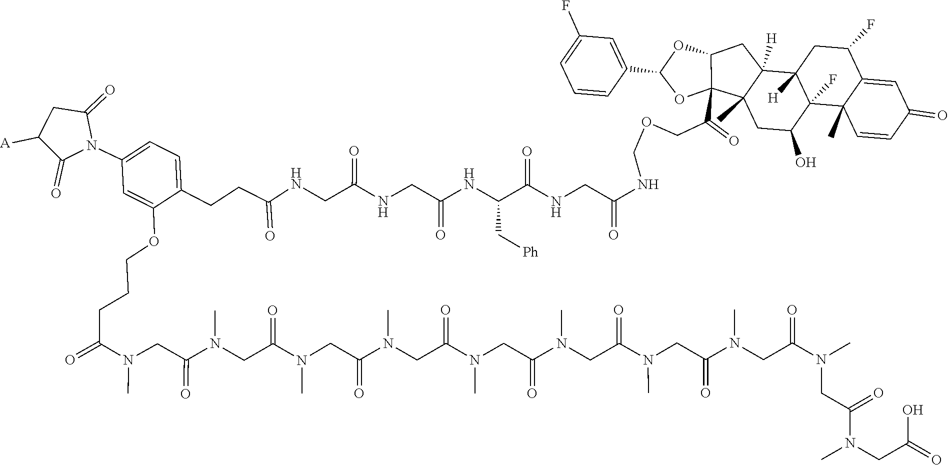 |
| II-48 | 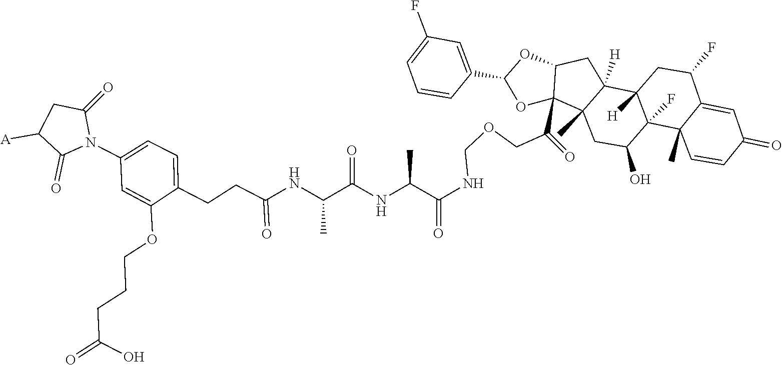 |
| II-49 | 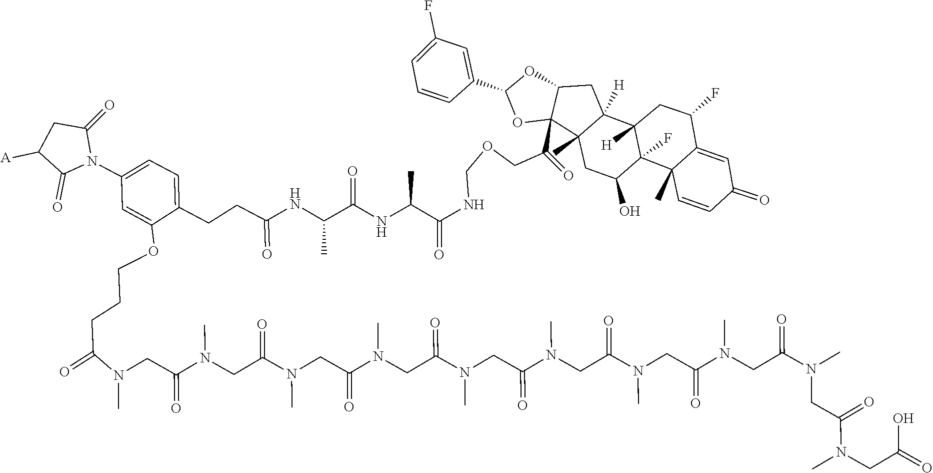 |
| II-50 | 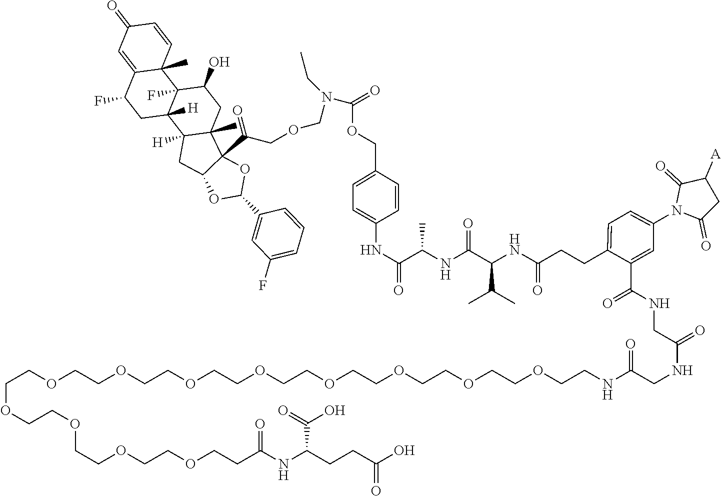 |
| II-51 | 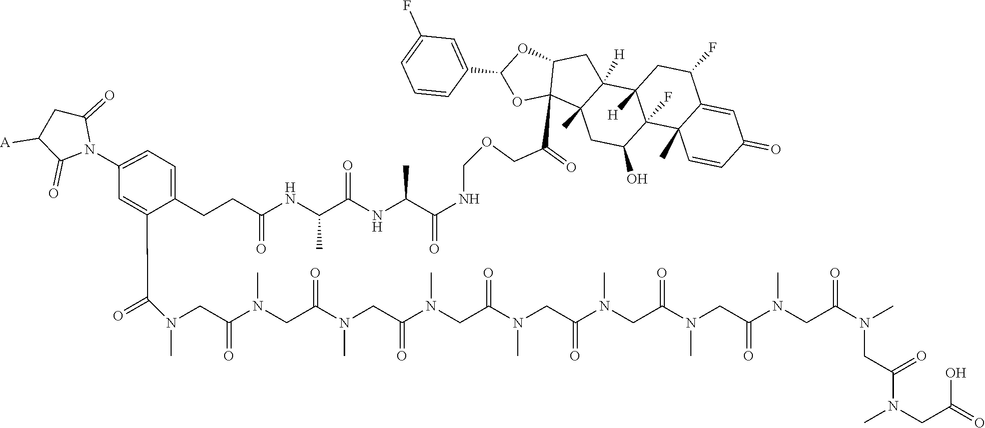 |
| II-52 | 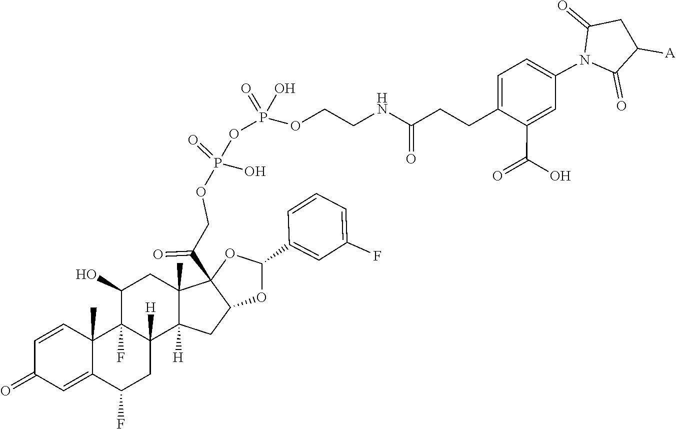 |
| II-53 | 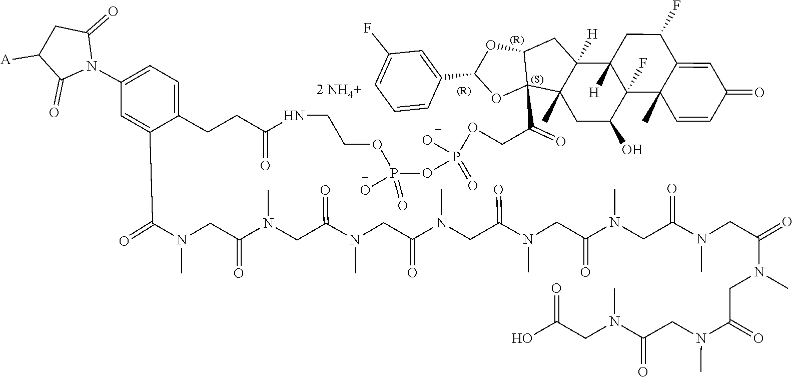 |
| II-54 | 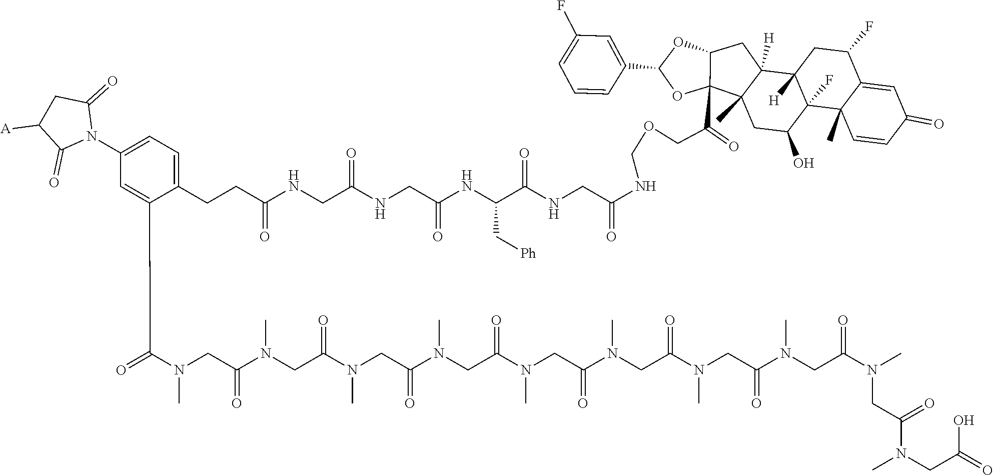 |
| II-55 | 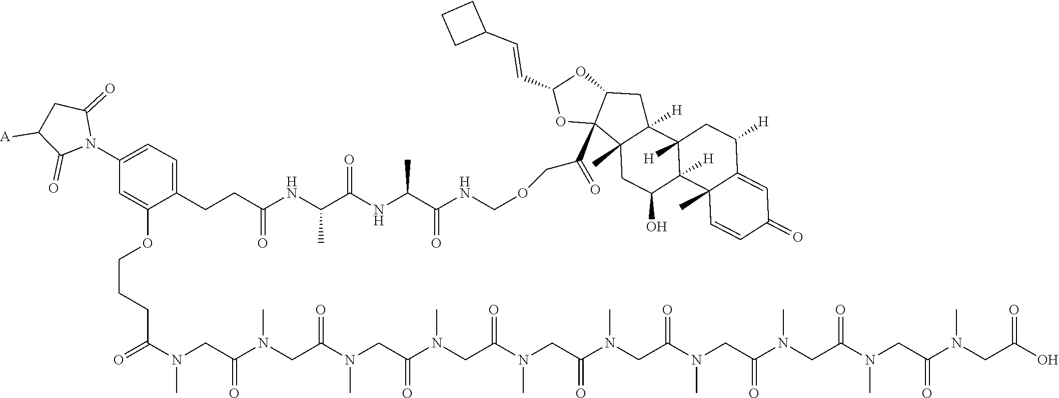 |
| II-56 | 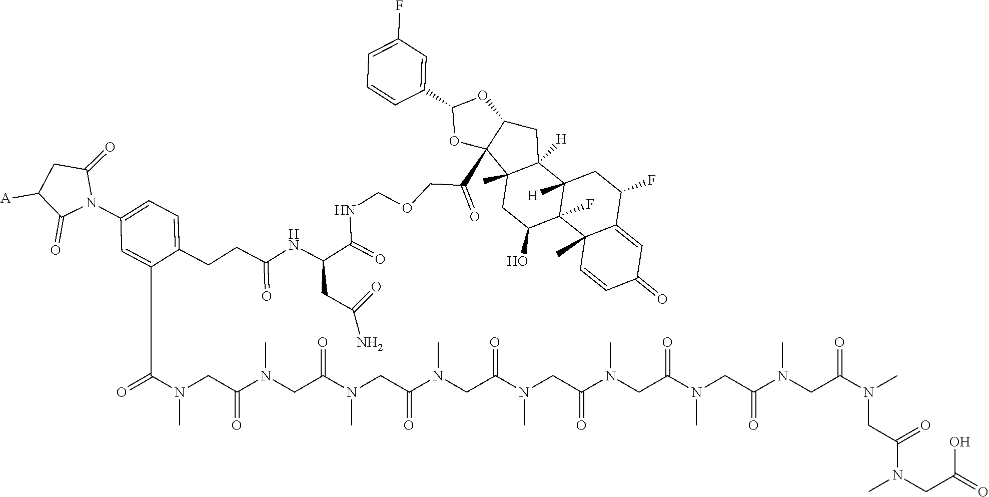 |
| II-57 | 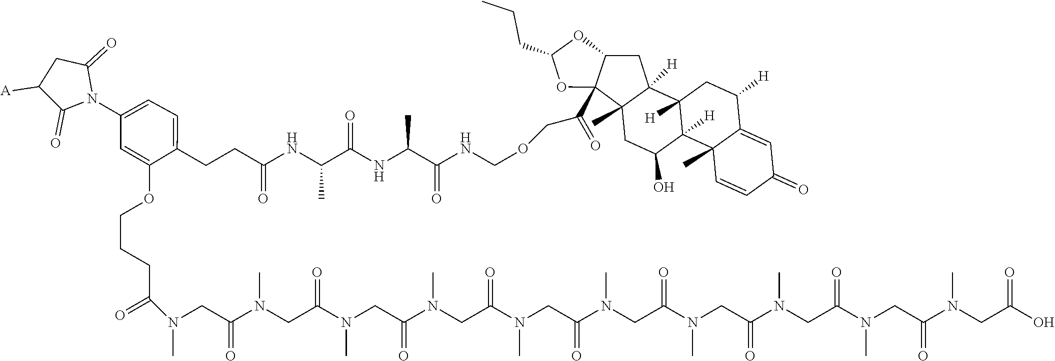 |
| II-58 | 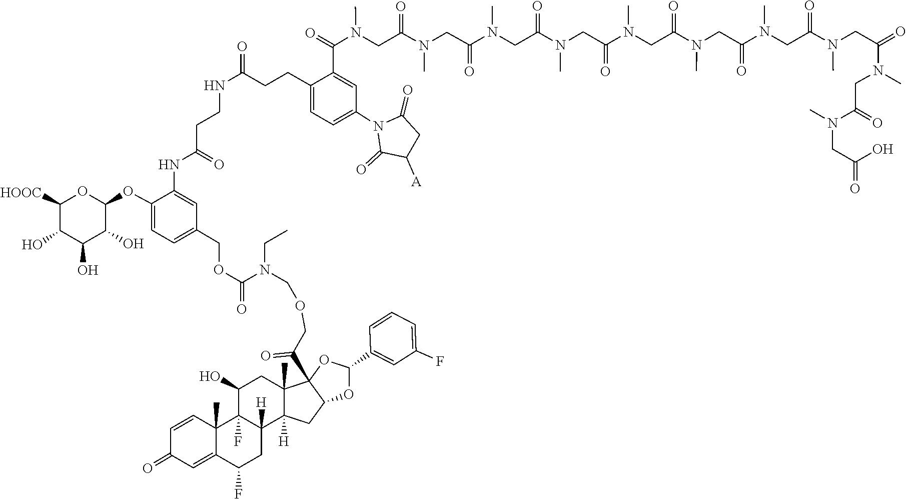 |
| II-59 | 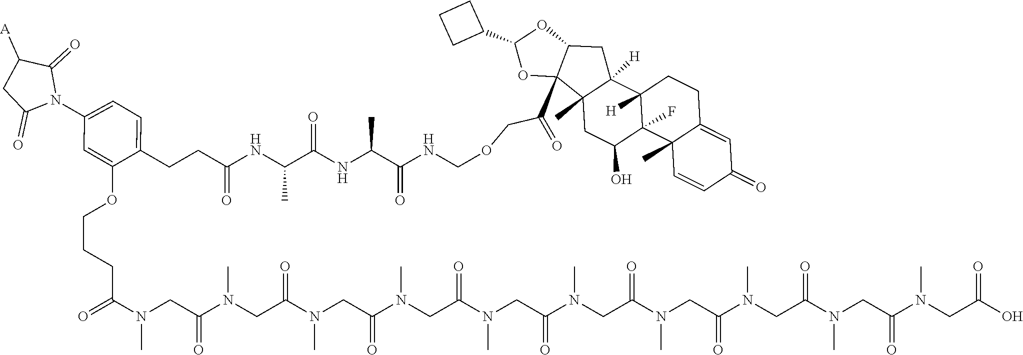 |
| II-60 | 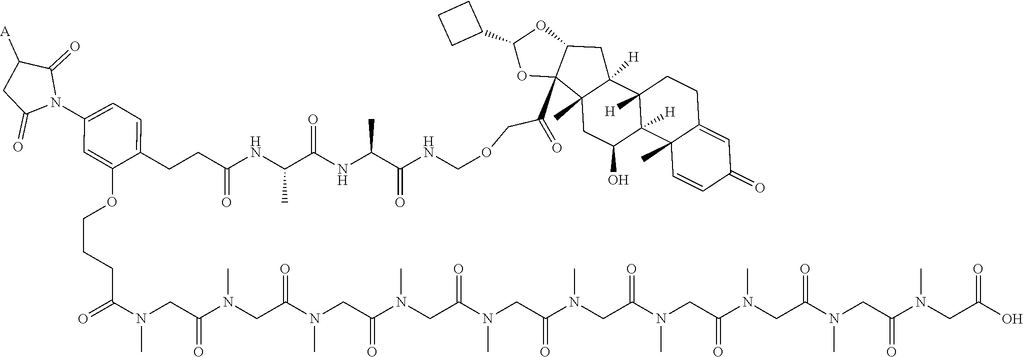 |
| II-61 | 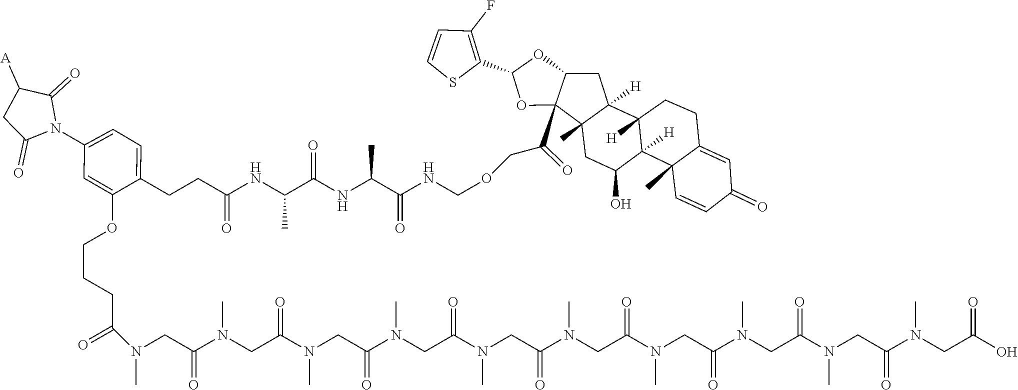 |
| II-62 | 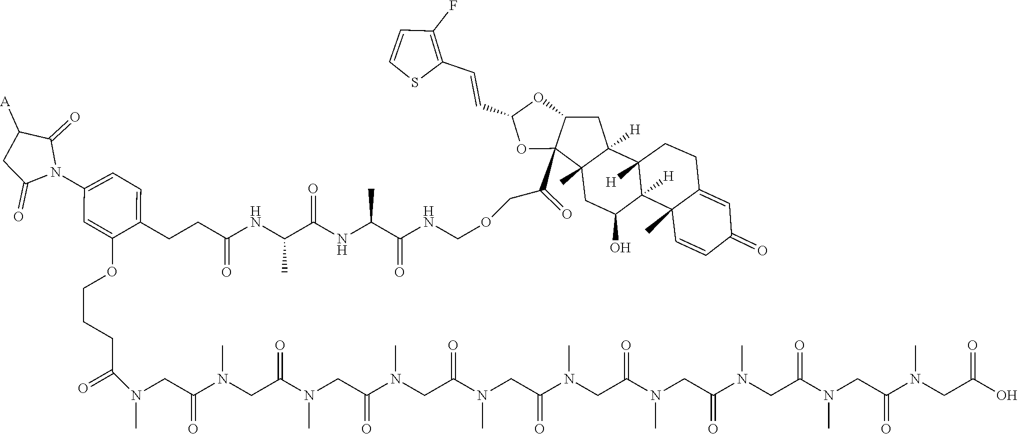 |
| II-63 | 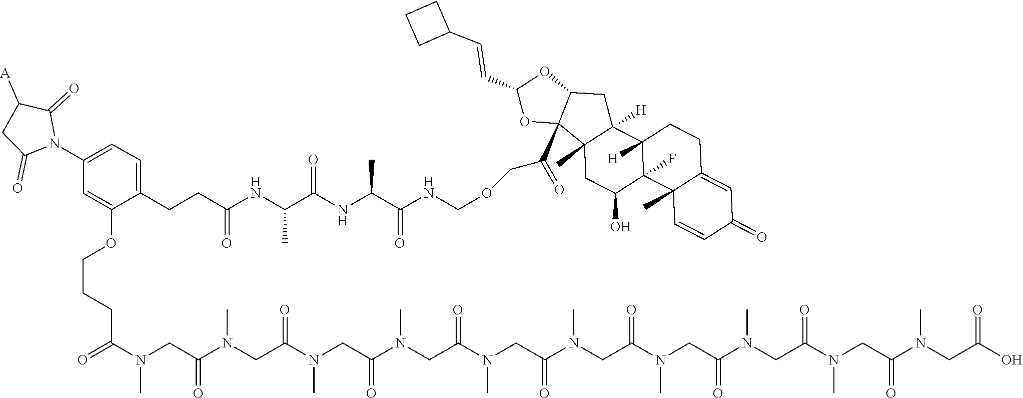 |
| II-64 | 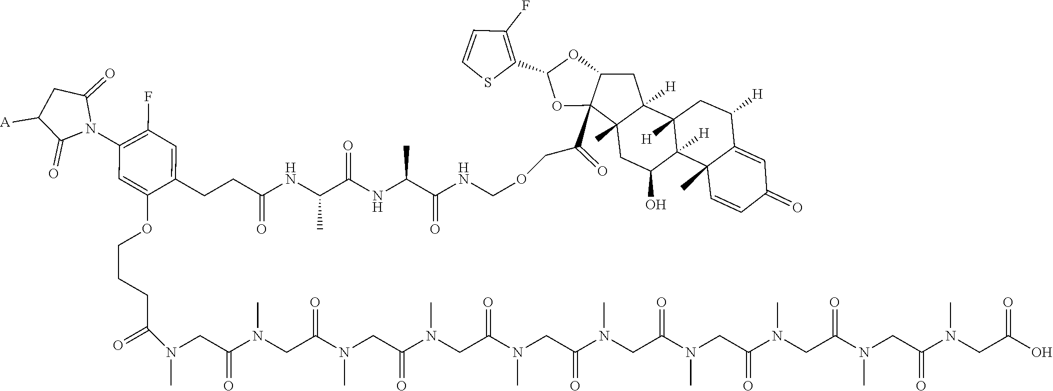 |
| II-65 | 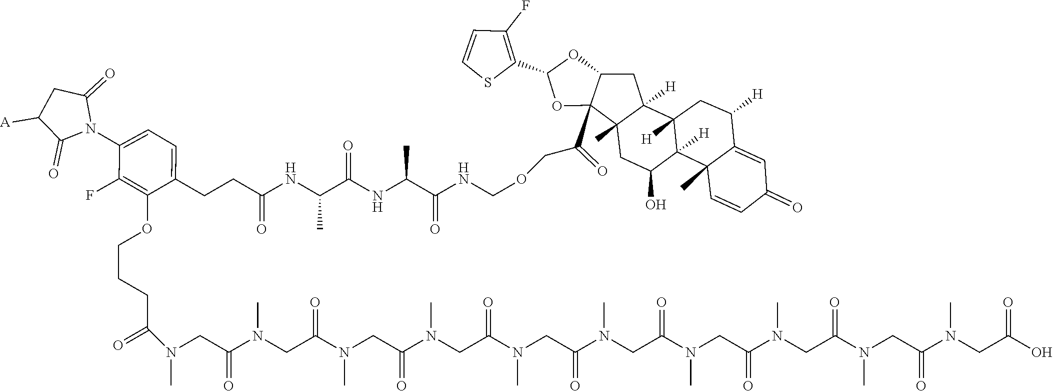 |
| II-66 | 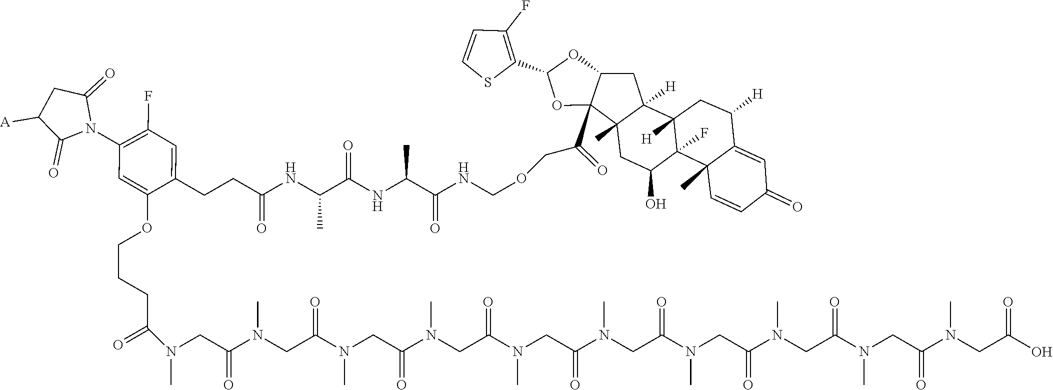 |
| II-67 | 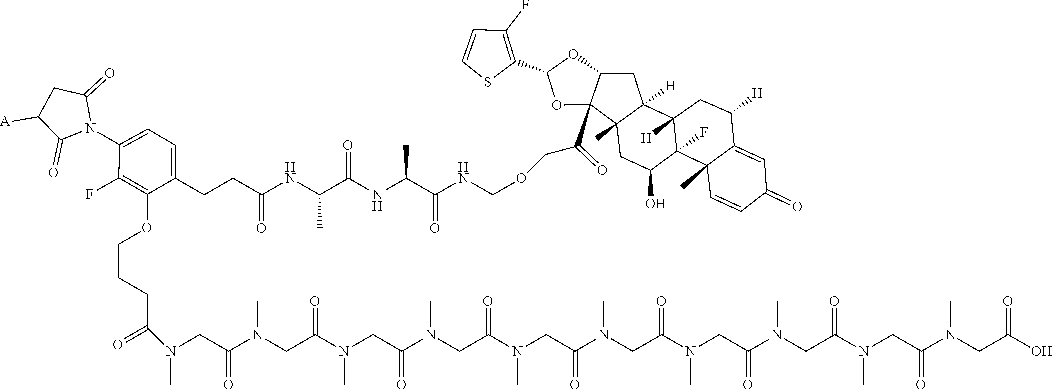 |
| II-68 | 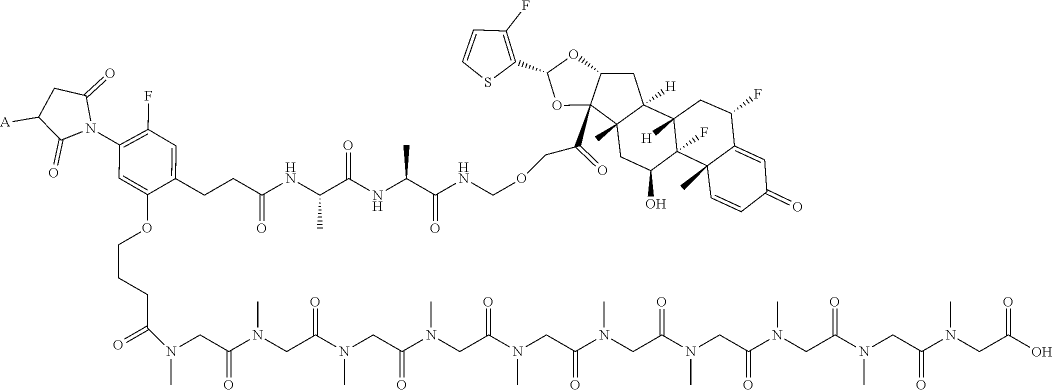 |
| II-69 | 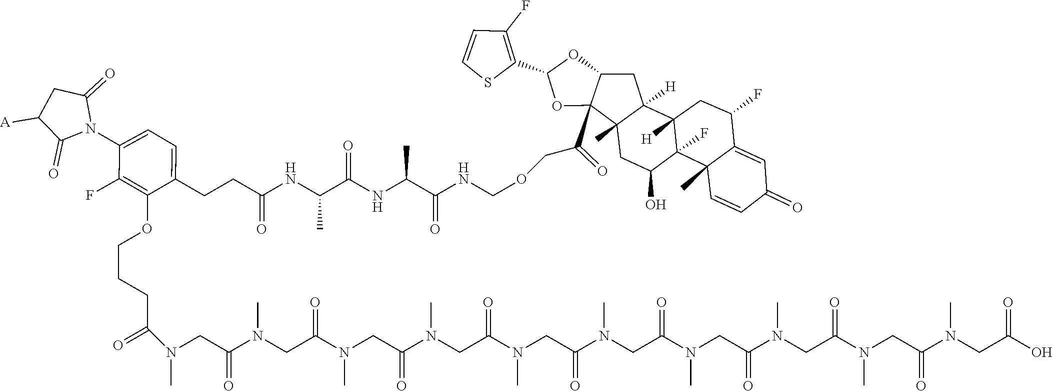 |
| II-70 | 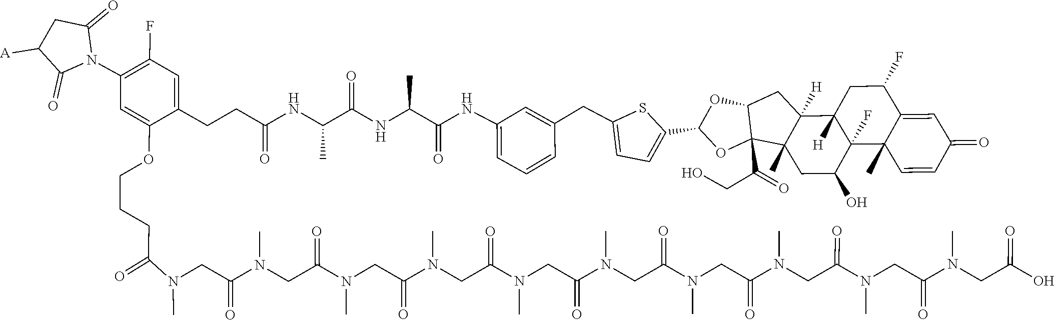 |
| II-71 | 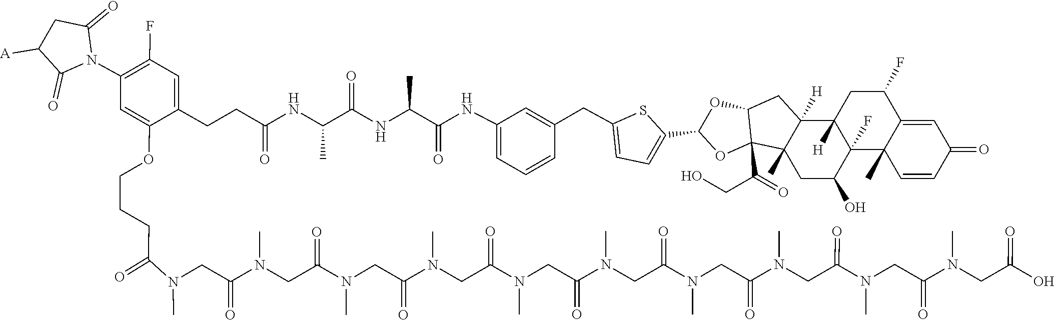 |
| II-72 | 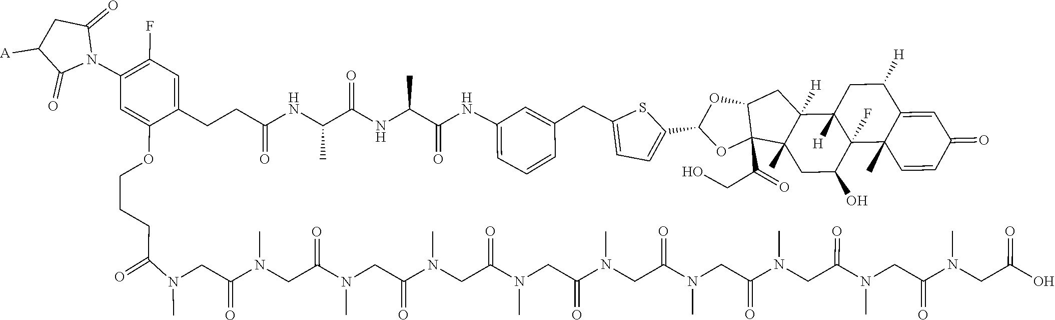 |
| II-73 | 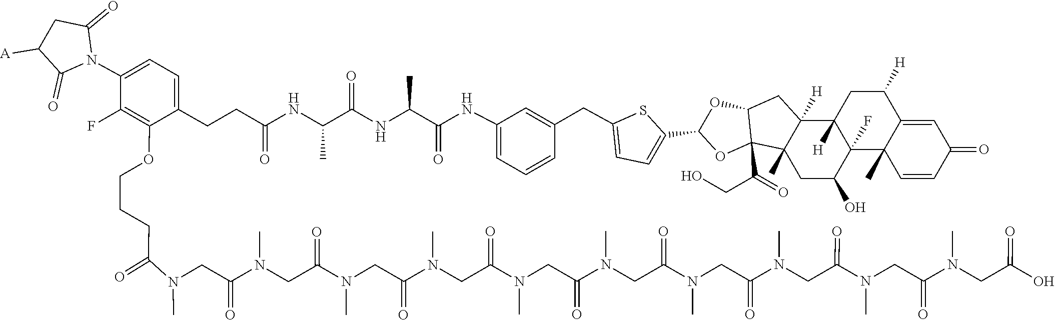 |
| II-74 | 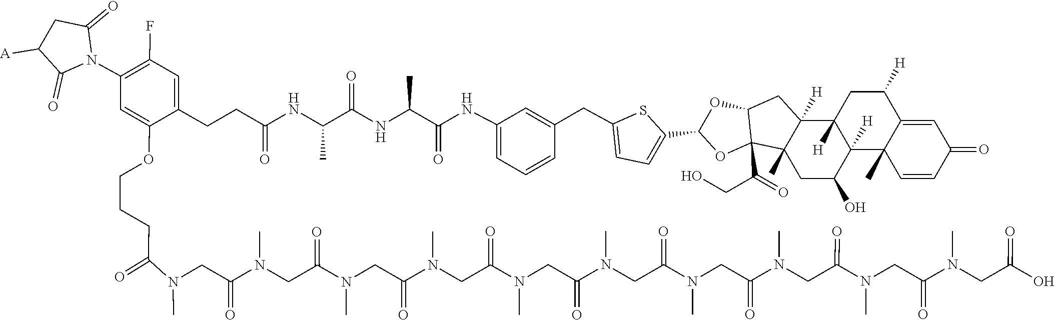 |
| II-75 | 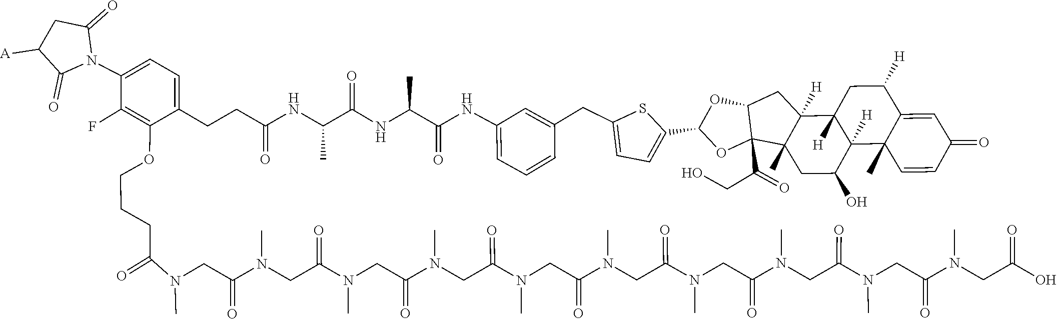 |
| II-76 | 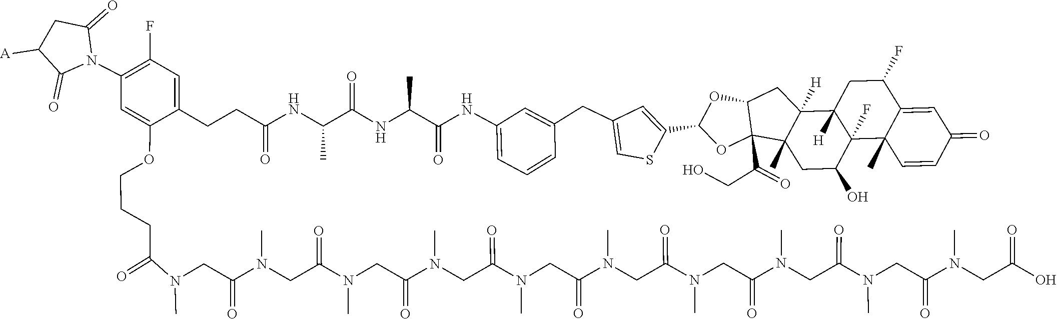 |
| II-77 | 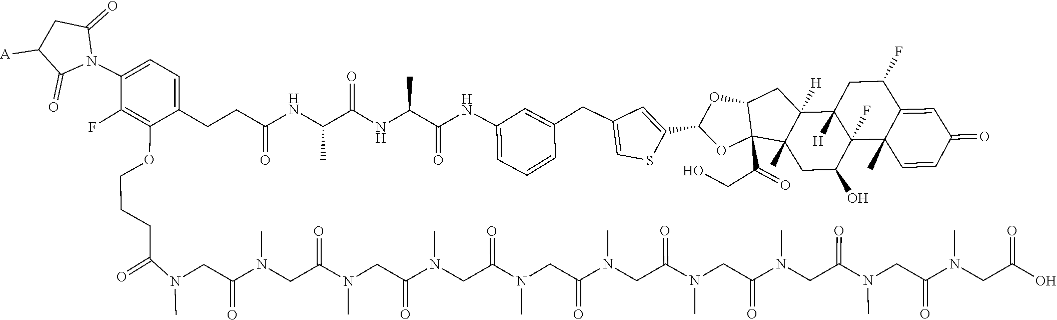 |

In some embodiments, a targeting moiety is an antibody. An antibody may be of any class, e.g., IgA, IgD, IgE, IgG, and IgM. Several of these classes may be further subdivided into isotypes, e.g., IgG1, IgG2, IgG3, IgG4, IgA1, and IgA2. The heavy-chain constant regions (Fc) that corresponds to the different classes of immunoglobulins may be α, δ, ε, γ, or μ. The light chains may be one of either kappa (κ) or lambda (λ), based on the amino acid sequences of the constant domains. Antibody constructs may also include an antibody fragment or a recombinant form thereof, including single chain variable fragments (scFvs).

In particular embodiments, an antibody construct or targeting moiety may comprise an antigen-binding antibody fragment. An antibody fragment may include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; and (iii) a Fv fragment consisting of the VL and VH domains of a single arm of an antibody. Although the two domains of the Fv fragment, VL and VH, may be coded for by separate genes, they may be linked by a synthetic linker to be made as a single protein chain in which the VL and VH regions pair to form monovalent molecules.

In other embodiments, an antibody construct or targeting moiety may contain, for example, two, three, four, five, six, seven, eight, nine, ten, or more antigen binding domains. An antibody construct or targeting moiety may contain two antigen binding domains in which each antigen binding domain can recognize the same antigen. An antibody construct or targeting moiety may contain two antigen binding domains in which each antigen binding domain can recognizes a different antigen. In some embodiments, an antibody construct or targeting moiety may comprise an Fc fusion protein. In further embodiments, the antibody construct comprises an antigen binding domain and an Fc region or domain. In still further embodiments, the antibody is a chimeric, humanized, or human antibody.

As used throughout this disclosure, “human antibodies” can include antibodies having, for example, the amino acid sequence of a human immunoglobulin and include antibodies isolated from human immunoglobulin libraries or from animals transgenic for one or more human immunoglobulins and that typically do not express endogenous immunoglobulins. Human antibodies can be produced using transgenic mice incapable of expressing functional endogenous immunoglobulins, but capable of expressing human immunoglobulin genes. Completely human antibodies that recognize a selected epitope can be generated using guided selection. In this approach, a selected non-human monoclonal antibody, e.g., a mouse antibody, is used to guide the selection of a completely human antibody recognizing the same epitope.

An antibody or antigen binding fragment thereof described herein can be derivatized or otherwise modified. For example, derivatized antibodies can be modified by glycosylation, acetylation, pegylation, phosphorylation, amidation, derivatization by known protecting/blocking groups, proteolytic cleavage, or the like.

An antibody, antibody construct, or targeting moiety may be a derivatized antibody. For example, derivatized antibodies may be modified by glycosylation, acetylation, pegylation, phosphorylation, amidation, derivatization by known protecting/blocking groups, proteolytic cleavage, linkage to a cellular ligand or other protein.

An antibody construct may comprise a light chain of an amino acid sequence having at least one, two, three, four, five, six, seven, eight, nine or ten modifications and in some embodiments, not more than 40, 35, 30, 25, 20, 15 or 10 modifications of the amino acid sequence relative to the natural or original amino acid sequence. An antibody construct may comprise a heavy chain of an amino acid sequence having at least one, two, three, four, five, six, seven, eight, nine or ten modifications and in some embodiments, not more than 40, 35, 30, 25, 20, 15 or 10 modifications of the amino acid sequence relative to the natural or original amino acid sequence.

In any of the embodiments disclosed herein, an antigen binding domain may specifically bind to a tumor antigen. In some embodiments a tumor antigen is ASGR2, LRRC15, mesothelin (MSLN), HER2, CEA, TROP2, EPHA2, p-cadherin, UPK1B, FOLH1, LYPD3, or PVRL4 (Nectin-4). An antigen binding domain may specifically bind to a molecule on an antigen presenting cell (APC). In certain embodiments, the antigen binding domain specifically binds to a human tumor antigen.

In some embodiments, an antibody (or antigen binding domain thereof) specifically binds to ASGR1, CTLA4 (also known as CD152), PD-1 (or CD279), PD-L1 (or CD274), TNFR2 (or TNFRSF1B), OX40 (or TNFRSF4), CD27, IL2RA, TNFRSF18, LAG-3, GARP, 4-1BB, ICOS, CD70, PDGFRβ, CD73, CD38, Integrin αvβ, CD248, FAP, Integrin αv, or Integrin αvβ6.

In some related embodiments, a targeting moiety is an antibody (e.g., pertuzumab, brentuximab, gemtuzumab, trastuzumab, inotuzumab, polatuzumab, enfortumab, trastuzumab, sacituzumab, belantamab, or moxetumomab).

In some embodiments, a targeting moiety is an anti-CD40 antibody, an antibody selected from an anti-LRRC15 antibody, an anti-CTSK antibody, an anti-ADAM12 antibody, an anti-ITGA11 antibody, an anti-FAP antibody, an anti-NOX4 antibody, an anti-SGCD antibody, an anti-SYNDIG1 antibody, an anti-CDH11 antibody, an anti-PLPP4 antibody, an anti-SLC24A2 antibody, an anti-PDGFRB antibody, an anti-THY1 antibody, an anti-ANTXR1 antibody, an anti-GAS1 antibody, an anti-CALHM5 antibody, an anti-SDC1 antibody, an anti-HER2 antibody, an anti-TROP2 antibody, an anti-MSLN antibody, an anti-Nectin4 antibody, an anti-ASGR1 antibody, an anti-Nectin-4 antibody, and an anti-MUC16 antibody.

In some embodiments, a targeting moiety is an anti-MSR1 antibody (e.g., as disclosed in PCT Publication No. WO 2019/217591, which is incorporated herein by reference)

Other exemplary antibodies within the scope of this disclosure will be apparent to those of ordinary skill in the art. For example, exemplary targeting moieties (e.g., antibodies) can be found in PCT Publication Nos. WO 2019/217591, WO 2018/089373, PCT/US2021/054296, or U.S. Pat. No. 11,179,473.

An antibody construct can be conjugated to a linker via cysteine-based bio-conjugation. An antibody construct can be exchanged into an appropriate buffer, for example, phosphate, borate, PBS, histidine, Tris-Acetate at a concentration of about 2 mg/mL to about 10 mg/mL with an appropriate number of equivalents of a reducing agent, for example, dithiothreitol (DTT) or tris(2-carboxyethyl)phosphine (TCEP). The resultant solution can be stirred for an appropriate amount of time and temperature to effect the desired reduction. A compound of Structure (I) can be added as a solution with stirring. Dependent on the physical properties of the compound of Structure (I), a co-solvent can be introduced prior to the addition of the compound of Structure (I) to facilitate solubility. The reaction can be stirred at room temperature for about 1 hour to about 12 hours depending on the observed reactivity. The progression of the reaction can be monitored by liquid chromatography-mass spectrometry (LC-MS). Once the reaction is deemed complete, the remaining free compound of Structure (I) can be removed by applicable methods and the conjugate of Structure (II) can be exchanged into the desired formulation buffer. Such conjugates can be synthesized starting with a targeting moiety (e.g., an antibody or mAb) and compound of Structure (I), e.g., 7 equivalents, using the conditions described in the Conjugation Reaction Scheme below. Monomer content and drug-antibody ratios can be determined by methods described herein and known in the art.

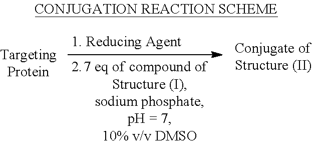

### Pharmaceutical Compositions

The compositions and methods described herein may be considered useful as pharmaceutical compositions for administration to a subject in need thereof. Pharmaceutical compositions may comprise at least the compositions described herein and one or more pharmaceutically acceptable carriers, diluents, excipients, stabilizers, dispersing agents, suspending agents, and/or thickening agents. The composition may comprise the conjugate of Structure (II). In some embodiments, a targeting moiety is an anti-LRRC15 antibody. In some embodiments, a targeting moiety is an anti-ASGR1 antibody. A pharmaceutical composition can comprise the conjugate of Structure (II) and one or more of buffers, antibiotics, steroids, carbohydrates, drugs (e.g., chemotherapy drugs), radiation, polypeptides, chelators, adjuvants and/or preservatives.

Pharmaceutical compositions may be formulated using one or more physiologically-acceptable carriers comprising excipients and auxiliaries. Formulation may be modified depending upon the route of administration chosen. Pharmaceutical compositions comprising a conjugate may be manufactured, for example, by lyophilizing the conjugate, mixing, dissolving, emulsifying, encapsulating or entrapping the conjugate. The pharmaceutical compositions may also include the conjugates in a free-base form or pharmaceutically-acceptable salt form.

Methods for formulation of the conjugates may include formulating any of the conjugates with one or more inert, pharmaceutically acceptable excipients or carriers to form a solid, semi-solid, or liquid composition. Solid compositions may include, for example, powders, tablets, dispersible granules and capsules, and in some embodiments, the solid compositions further contain nontoxic, auxiliary substances, for example wetting or emulsifying agents, pH buffering agents, and other pharmaceutically acceptable additives. Alternatively, the conjugates of Structure (II) may be lyophilized or in powder form for re-constitution with a suitable vehicle, e.g., sterile pyrogen-free water, before use.

Pharmaceutical compositions of the conjugates of Structure (II) may comprise at least one active ingredient (e.g., a compound, salt or conjugate and other agents). The active ingredients may be entrapped in microcapsules prepared, for example, by co-acervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

Pharmaceutical compositions as often further may comprise more than one active compound as necessary for the particular indication being treated. The active compounds may have complementary activities that do not adversely affect each other. For example, the composition may further comprise a chemotherapeutic agent, cytotoxic agent, cytokine, growth-inhibitory agent, anti-hormonal agent, anti-angiogenic agent, and/or cardioprotectant. Such molecules may be present in combination in amounts that are effective for the purpose intended.

The compositions and formulations may be sterilized. Sterilization may be accomplished by filtration through sterile filtration.

The compositions may be formulated for administration as an injection. Non-limiting examples of formulations for injection may include a sterile suspension, solution or emulsion in oily or aqueous vehicles. Suitable oily vehicles may include lipophilic solvents or vehicles such as fatty oils or synthetic fatty acid esters, or liposomes. Aqueous injection suspensions may contain substances which increase the viscosity of the suspension. The suspension may also contain suitable stabilizers. Injections may be formulated for bolus injection or continuous infusion. Alternatively, the compositions may be lyophilized or in powder form for reconstitution with a suitable vehicle, e.g., sterile pyrogen-free water, before use.

The phrases “parenteral administration” and “administered parenterally” as used throughout this disclosure means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used throughout this disclosure means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (a) sugars, such as lactose, glucose and sucrose; (b) starches, such as corn starch and potato starch; (c) cellulose, and its derivatives, such as sodium carboxymethyl cellulose, ethyl cellulose and cellulose acetate; (d) powdered tragacanth; (e) malt; (f) gelatin; (g) talc; (h) excipients, such as cocoa butter and suppository waxes; (i) oils, such as peanut oil, cottonseed oil, safflower oil, sesame oil, olive oil, corn oil and soybean oil; (j) glycols, such as propylene glycol; (k) polyols, such as glycerin, sorbitol, mannitol and polyethylene glycol; (l) esters, such as ethyl oleate and ethyl laurate; (m) agar; (n) buffering agents, such as magnesium hydroxide and aluminum hydroxide; (o) alginic acid; (p) pyrogen-free water; (q) isotonic saline; (r) Ringer's solution; (s) ethyl alcohol; (t) phosphate buffer solutions; and (u) other non-toxic compatible substances employed in pharmaceutical formulations.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, or the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, or the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, or the like. Organic bases from which salts can be derived include, for example, primary, secondary, and tertiary amines, substituted amines including naturally occurring substituted amines, cyclic amines, basic ion exchange resins, or the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

For parenteral administration, the compounds, salts or conjugates may be formulated in a unit dosage injectable form (e.g., solution, suspension, emulsion) in association with a pharmaceutically acceptable parenteral vehicle. Such vehicles may be inherently non-toxic, and non-therapeutic. Vehicles may be water, saline, Ringer's solution, dextrose solution, and 5% human serum albumin. Non-aqueous vehicles such as fixed oils and ethyl oleate may also be used. Liposomes may be used as carriers. The vehicle may contain minor amounts of additives such as substances that enhance isotonicity and chemical stability (e.g., buffers and preservatives).

Sustained-release preparations may be also be prepared. Examples of sustained-release preparations may include semipermeable matrices of solid hydrophobic polymers that may contain the compound, salt or conjugate, and these matrices may be in the form of shaped articles (e.g., films or microcapsules). Examples of sustained-release matrices may include polyesters, hydrogels (e.g., poly(2-hydroxyethyl-methacrylate), or poly(vinyl alcohol)), polylactides, copolymers of L-glutamic acid and γ ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations may be prepared for storage by mixing conjugates of Structure (II) with a pharmaceutically acceptable carrier, excipient, and/or a stabilizer. This formulation may be a lyophilized formulation or an aqueous solution. Acceptable carriers, excipients, and/or stabilizers may be nontoxic to recipients at the dosages and concentrations used. Acceptable carriers, excipients, and/or stabilizers may include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

Pharmaceutical formulations of the conjugates may have an average payload-antibody construct ratio (“DAR”) selected from about 1 to about 20, from about 1 to about 10, from about 1 to about 5, from about 1 to about 2, or from about 1 to about 1. In some embodiments, the average DAR of the formulation is from about 2 to about 8, from about 3 to about 8, from about 3 to about 7, from about 3 to about 5, or about 2. In some embodiments, a pharmaceutical formulation has an average DAR of about 3, about 3.5, about 4, about 4.5 or about 5.

### Method of Treatment

Conjugates of the present disclosure are useful for treating disease (i.e., conjugates of Structure (II)). Those conjugates disclosed herein offer a targeted approach to drug delivery strategies. Accordingly, certain embodiments provide a method of treating a disease (or the symptoms thereof) comprising administering to a mammal (e.g., a human) in need thereof a therapeutically effective amount of or conjugate of Structure (II) or a pharmaceutical composition comprising the same.

Treat and/or treating refer to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition or the like, are experienced by a patient. Treat can be used herein to refer to a method that results in some level of treatment or amelioration of the disease or condition, and can contemplate a range of results directed to that end, including but not restricted to prevention of the condition entirely.

Prevent, preventing or the like refer to the prevention of the disease or condition, e.g., tumor formation, in the patient. For example, if an individual at risk of developing a tumor or other form of cancer is treated with the methods of this disclosure and does not later develop the tumor or other form of cancer, then the disease has been prevented, at least over a period of time, in that individual. Preventing can also refer to preventing re-occurrence of a disease or condition in a patient that has previously been treated for the disease or condition, e.g., by preventing relapse.

A therapeutically effective amount (also referred to as an effective amount) can be the amount of a composition comprising a conjugate of Structure (II) sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment and can be ascertainable by one skilled in the art using known techniques and the teachings provided herein.

The conjugates that can be used in therapy can be formulated and dosages established in a fashion consistent with good medical practice taking into account the disease or condition to be treated, the condition of the individual patient, the site of delivery of the composition, the method of administration and other factors known to practitioners. The compositions can be prepared according to the description of preparation described herein.

Pharmaceutical compositions can be used in the methods described herein and can be administered to a subject in need thereof using a technique known to one of ordinary skill in the art which can be suitable as a therapy for the disease or condition affecting the subject. One of ordinary skill in the art would understand that the amount, duration and frequency of administration of a pharmaceutical composition to a subject in need thereof depends on several factors including, for example, the health of the subject, the specific disease or condition of the patient, the grade or level of a specific disease or condition of the patient, the additional treatments the subject is receiving or has received, or the like.

The conjugates, compositions, and methods of this disclosure are useful in the treatment or prevention of disease, such as infection, autoimmune disorders (e.g., multiple sclerosis), inflammation (including autoinflammatory disorders), and cancer as single agents. Alternatively, the conjugates, compositions, and methods of this disclosure may be used in combination therapies with second therapeutic agents for treating or preventing diseases, such as infection, autoimmune disorders (e.g., multiple sclerosis), inflammation (including autoinflammatory disorders), and cancer.

Some embodiments provide a pharmaceutical composition comprising the conjugate of Structure (II) and a pharmaceutically acceptable excipient.

Another embodiment provides a method for treatment of cancer comprising administering an effective amount of the conjugate of Structure (II) or a pharmaceutical composition thereof to a subject in need thereof. Still another embodiment provides a method for treatment of infection, inflammation, an autoimmune disorder, or combinations thereof, the method comprising administering an effective amount of the conjugate of Structure (II) or a pharmaceutical composition thereof to a subject in need thereof. In some of the foregoing embodiments, the effective amount of the conjugate is administered intravenously, subcutaneously, or intratumorally.

Antigens targeted by conjugates of Structure (II) may be derived from the following specific conditions and/or families of conditions, including cancers such as cancer associated fibroblasts (CAF), brain cancers, skin cancers, lymphomas, sarcomas, lung cancer, liver cancer, leukemia, uterine cancer, breast cancer (including triple negative breast cancer), ovarian cancer, cervical cancer, uterine cancer, bladder cancer, gastric cancer, esophageal cancer, kidney cancer, hemangiosarcomas, bone cancer, blood cancer, testicular cancer, prostate cancer, stomach cancer, colon cancer, intestinal cancer, pancreatic cancer, head and neck cancer (including head and neck squamous cell carcinoma), mesothelioma, melanoma, and other types of cancers as well as pre-cancerous conditions such as hyperplasia or the like.

Non-limiting examples of further cancers include acute lymphoblastic leukemia (ALL); acute myeloid leukemia (AML); adrenocortical carcinoma; astrocytoma, childhood cerebellar or cerebral; basal-cell carcinoma; Bone tumor, osteosarcoma/malignant fibrous histiocytoma; Brain cancer; Brain tumors (e.g., cerebellar astrocytoma, malignant glioma, ependymoma, medulloblastoma, visual pathway and hypothalamic glioma); Brainstem glioma; Breast cancer (including triple negative breast cancer); Bronchial adenomas/carcinoids; Burkitt's lymphoma; Cerebellar astrocytoma; Cervical cancer; Cholangiocarcinoma; Chondrosarcoma; Chronic lymphocytic leukemia; Chronic myelogenous leukemia; Chronic myeloproliferative disorders; Colon cancer; Cutaneous T-cell lymphoma; Endometrial cancer; Ependymoma; Esophageal cancer; Eye cancers, such as, intraocular melanoma and retinoblastoma; Gallbladder cancer; Gastric cancer; Glioma; Hairy cell leukemia; Head and neck cancer (including head and neck squamous cell carcinoma); Heart cancer; Hepatocellular (liver) cancer; Hodgkin lymphoma; Hypopharyngeal cancer; Islet cell carcinoma (endocrine pancreas); Kaposi sarcoma; Kidney cancer (renal cell cancer); Laryngeal cancer; Leukaemia, such as, acute lymphoblastic, acute myeloid, chronic lymphocytic, chronic myelogenous and, hairy cell; Lip and oral cavity cancer; Liposarcoma; Lung cancer, such as, non-small cell and small cell; Lymphoma, such as, AIDS-related, Burkitt; Lymphoma, cutaneous T-Cell, Hodgkin and Non-Hodgkin, Macroglobulinemia, Malignant fibrous histiocytoma of bone/osteosarcoma; Melanoma (including cutaneous melanoma); Merkel cell cancer; Mesothelioma; Multiple myeloma/plasma cell neoplasm; Mycosis fungoides; Myelodysplastic syndromes; Myelodysplastic/myeloproliferative diseases; Myeloproliferative disorders, chronic; Nasal cavity and paranasal sinus cancer; Nasopharyngeal carcinoma; Neuroblastoma; Oligodendroglioma; Oropharyngeal cancer; Osteosarcoma/malignant fibrous histiocytoma of bone; Ovarian cancer; Pancreatic cancer; Parathyroid cancer; Pharyngeal cancer; Pheochromocytoma; Pituitary adenoma; Plasma cell neoplasia; Pleuropulmonary blastoma; Prostate cancer; Rectal cancer; Renal cell carcinoma (kidney cancer); Renal pelvis and ureter, transitional cell cancer; Rhabdomyosarcoma; Salivary gland cancer; Sarcoma, Ewing family of tumors; Sarcoma, Kaposi; Sarcoma, soft tissue; Sarcoma, uterine; Sezary syndrome; Skin cancer (non-melanoma); Skin carcinoma; Small intestine cancer; Soft tissue sarcoma; Squamous cell carcinoma; Squamous neck cancer with occult primary, metastatic; Stomach cancer; Testicular cancer; Throat cancer; Thymoma and thymic carcinoma; Thymoma; Thyroid cancer; Thyroid cancer, childhood; Uterine cancer; Vaginal cancer; Waldenström macroglobulinemia; Wilms tumor, or any combination thereof.

Further therapeutic agents that can be combined with a conjugate of this disclosure are found in Goodman and Gilman's “The Pharmacological Basis of Therapeutics” Tenth Edition edited by Hardman, Limbird and Gilman or the Physician's Desk Reference, both of which are incorporated herein by reference in their entirety.

The conjugates of Structure (II) described herein can be used in combination with the agents disclosed herein or other suitable agents, depending on the condition being treated. Hence, in some embodiments the one or more conjugates of this disclosure will be co-administered with other agents as described herein. When used in combination therapy, the conjugates described herein are administered with the second agent simultaneously or separately. This administration in combination can include simultaneous administration of the two agents in the same dosage form, simultaneous administration in separate dosage forms, and separate administration. That is, a conjugate described herein and any of the agents described above can be formulated together in the same dosage form and administered simultaneously. Alternatively, a conjugate of this disclosure and any of the agents described above can be simultaneously administered, wherein both the agents are present in separate formulations. In another alternative, a conjugate of the present disclosure can be administered just followed by and any of the agents described above, or vice versa. In some embodiments of the separate administration protocol, a conjugate of this disclosure and any of the agents described above are administered a few minutes apart, or a few hours apart, or a few days apart.

In some embodiments of this disclosure, including those in which the conjugates, and pharmaceutical compositions of this disclosure are intended for the treatment or prevention of inflammation (including an autoinflammatory disorder) or an autoimmune disorder, the second therapeutic agent comprises an anti-inflammatory composition; a steroid composition, a nonsteroidal anti-inflammatory drug (NSAID) composition, a cyclooxygenase (COX) enzyme (e.g., COX1 and/or COX2 inhibitor) composition; or a regulatory T-cell antagonist composition.

In some embodiments of this disclosure, including those in which the conjugates and pharmaceutical compositions of this disclosure are intended for the treatment or prevention of cancer, the second therapeutic agent comprises one or more of a second conjugate or pharmaceutical composition of this disclosure; a chemotherapy composition; a radiation treatment; an immune conjugate composition having specificity for LRRC15, HER2 or MSLN; an immune conjugate composition having specificity for an antigen other than LRRC15, HER2, or MSLN; or a modified T-cell composition (e.g., a CAR-T and/or TCR-T composition).

In some embodiments, the methods of treatment provided herein comprise administering an additional therapeutic agent to the subject, such as an anti-cancer agent or anti-fibrosis agent. In some embodiments, the additional therapeutic agent is an anti-cancer agent selected from a chemotherapeutic agent, cytotoxic agent, cytokine, growth-inhibitory agent, anti-hormonal agent, anti-angiogenic agent, and/or cardioprotectant.

Examples of chemotherapeutic agents contemplated as further therapeutic agents include alkylating agents, such as nitrogen mustards (e.g., mechlorethamine, cyclophosphamide, ifosfamide (IFEX®), melphalan (Alkeran®), and chlorambucil); bifunctional chemotherapeutics (e.g., bendamustine); nitrosoureas (e.g., carmustine (BCNU, BiCNU®; polifeprosan 20 implant (Gliadel®)), lomustine (CCNU), and semustine (methyl-CCNU)); ethyleneimines and methyl-melamines (e.g., triethylenemelamine (TEM), triethylene thiophosphoramide (thiotepa), and hexamethylmelamine (HMM, altretamine)); alkyl sulfonates (e.g., busulfan (Myleran®), busulfan injection (Busulfex®)); and triazines (e.g., dacabazine (DTIC)); antimetabolites, such as folic acid analogues (e.g., methotrexate (Folex®), trimetrexate, and pemetrexed (multi-targeted antifolate)) and capecitabine (Xeloda®); pyrimidine analogues (such as 5-fluorouracil (5-FU, Adrucil®, Efudex®), fluorodeoxyuridine, tezacitabine, gemcitabine, cytosine arabinoside (AraC, cytarabine (Cytosar-U®); cytarabine liposome injection (DepoCyt®)), 5-azacytidine, and 2,2′-difluorodeoxycytidine); purine analogues (e.g., 6-mercaptopurine (Purinethol®), 6-thioguanine, azathioprine, 2′-deoxycoformycin (pentostatin), erythrohydroxynonyladenine (EHNA), fludarabine phosphate (Fludara®), 2 chlorodeoxyadenosine (cladribine, 2-CdA)); Type I topoisomerase inhibitors such as camptothecin (CPT), topotecan (Hycamptin®), and irinotecan (Camptosar®); natural products, such as epipodophylotoxins (e.g., etoposide (Vepesid®) and teniposide (Vumon®)); vinca alkaloids (e.g., vinblastine (Velban®), vincristine (Oncovin®), and vinorelbine (Navelbine®)); anti-tumor antibiotics such as actinomycin D (dactinomycin, Cosmegan®), doxorubicin hydrochloride (Adriamycin®, Rubex®), mitoxantrone (Novantrone®), and bleomycin sulfate (Blenoxane®); radiosensitizers such as 5-bromodeozyuridine, 5-iododeoxyuridine, and bromodeoxycytidine; platinum coordination complexes such as cisplatin (Platinol®), carboplatin (Paraplatin®), and oxaliplatin (Eloxatin®); substituted ureas, such as hydroxyurea (Hydrea®); microtubule inhibitors such as paclitaxel (Taxol®) and docetaxel (Taxotere®); immunosuppressive agents such as cyclophosphamide (Cytoxan® or Neosar®); hormone-based compound such as anastrozole (Arimidex®), exemestane (Aromasin®), letrozole (Femara®), fulvestrant (Faslodex®), and bicalutamide (Casodex®) and tamoxifen citrate (Nolvadex®); an anti-inflammatory agent such as dexamethasone; an anti-androgen compound such as flutamide (Eulexin®); an anthracycline compound such as idarubicin (Idamycin®, Zavedos®) and epirubicin (Ellence®); bioreductive anti-cancer agent such as tirapazamine (Tirazone®); serine/threonine kinase inhibitors such as CDK4/6 inhibitors abemaciclib (Verzenio®), palbociclib (Ibrance®), and ribociclib (Kisqali®); and methylhydrazine derivatives such as N methylhydrazine (MIH) and procarbazine.

### Method of Preparation

The examples and preparations provided below further illustrate and exemplify the compounds of the present disclosure and methods of preparing such compounds and conjugates thereof. It is to be understood that the scope of the present disclosure is not limited in any way by the scope of the following examples and preparations. In the following examples, and throughout the specification and claims, molecules and moieties with a single stereocenter, unless otherwise noted, exist as a racemic mixture. Those molecules and moieties with two or more stereocenters, unless otherwise noted, exist as a racemic mixture of diastereomers. Single enantiomers/diastereomers may be obtained by methods known to those skilled in the art.

It will also be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, or the like. Suitable protecting groups for amino, amidino and guanidino include t-butoxycarbonyl, benzyloxycarbonyl, or the like. Suitable protecting groups for mercapto include —C(O)—R″ (where R″ is alkyl, aryl or arylalkyl), p-methoxybenzyl, trityl or the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3^{rd} Ed., Wiley. As one of skill in the art would appreciate, the protecting group may also be a polymer resin such as a Wang resin, Rink resin, or a 2-chlorotrityl-chloride resin.

Furthermore, all compounds of this disclosure which exist in free base or acid form can be converted to their salts by treatment with the appropriate inorganic or organic base or acid by methods known to one skilled in the art. Salts of the compounds of this disclosure can be converted to their free base or acid form by standard techniques.

The following Reaction Scheme and Examples illustrate exemplary methods of making compounds of this disclosure. It is understood that one skilled in the art may be able to make these compounds by similar methods or by combining other methods known to one skilled in the art. It is also understood that one skilled in the art would be able to make, in a similar manner as described below, other compounds of Structure (I) not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, e.g., Advanced Organic Chemistry: Reactions, Mechanisms, and Structure, 5^{th} edition (Wiley, December 2000)) or prepared as described in this disclosure. The compound 4-((S)-2-((S)-2-amino-3-methylbutanamido)-5-ureidopentanamido)benzyl 3-(2-amino-4-(dipropylcarbamoyl)-3H-benzo[b]azepine-8-carboxamido)-7,8-dihydro-1,6-naphthyridine-6(5H)-carboxylate can be synthesized according to U.S. Pat. No. 10,239,862, the synthetic procedure for this compound is hereby incorporated by reference.

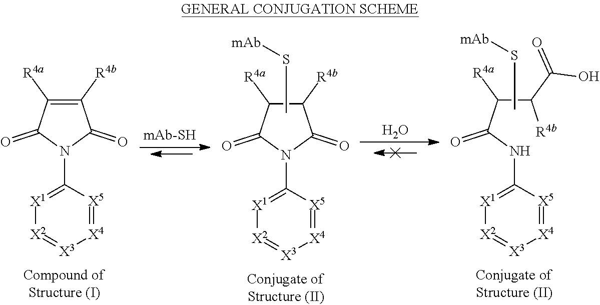

The General Reaction Scheme above is illustrative of a conjugation reaction between a compound of Structure (I) and a targeting moiety or binding fragment thereof wherein mAb-SH represents the targeting moiety or binding fragment thereof (e.g., a monoclonal antibody) having a free thiol (—SH), and X¹, X², X², X⁴, X⁵, R^{4a}, and R^{4b} are as defined herein. A targeting moiety or binding fragment thereof having a free thiol can be prepared and conjugated to a compound of Structure (I) using methods known in the art and as described herein (see, e.g., Conjugation Example 1). Following an initial conjugation reaction, the resultant conjugate of Structure (II) may undergo an irreversible hydrolysis reaction to form an alternative conjugate of Structure (II) as shown.

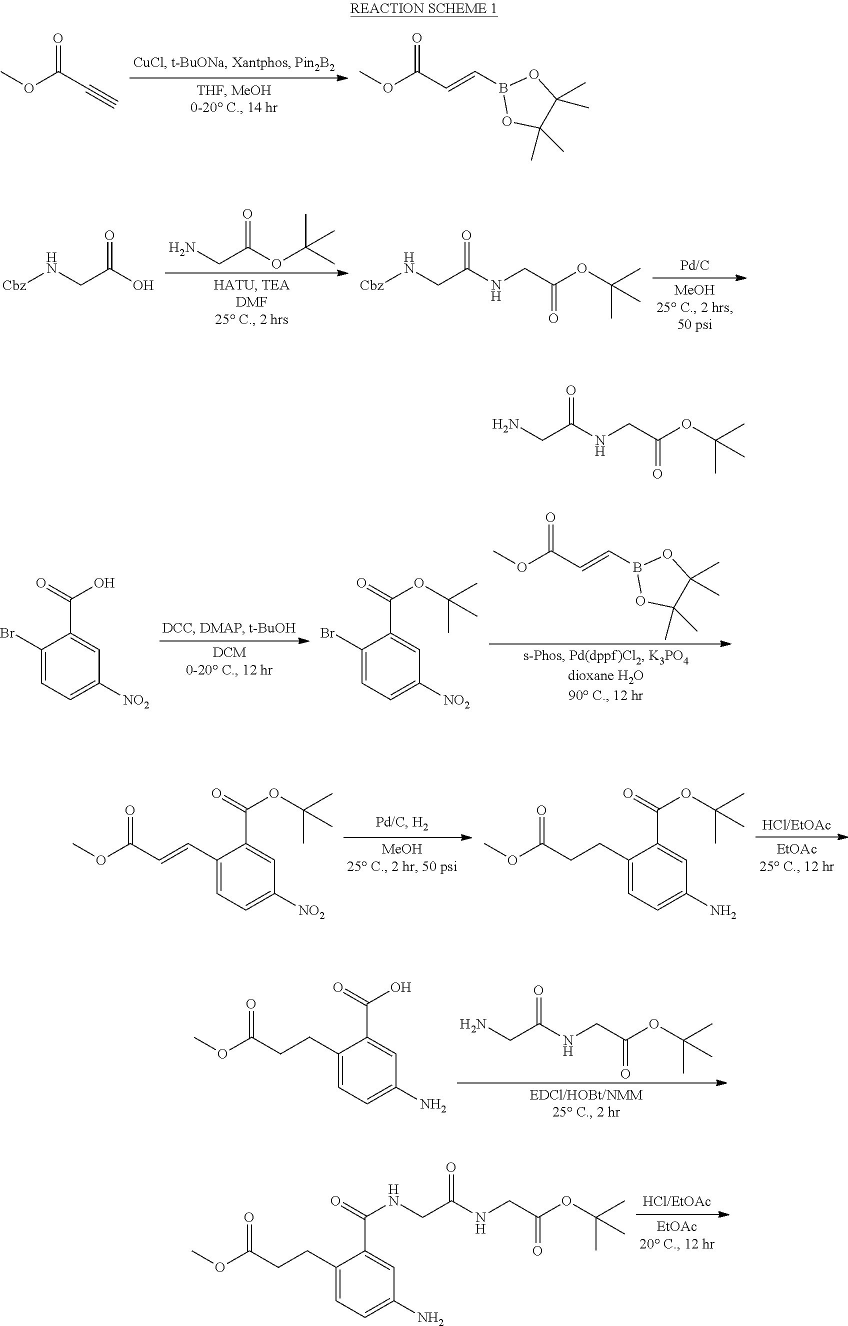

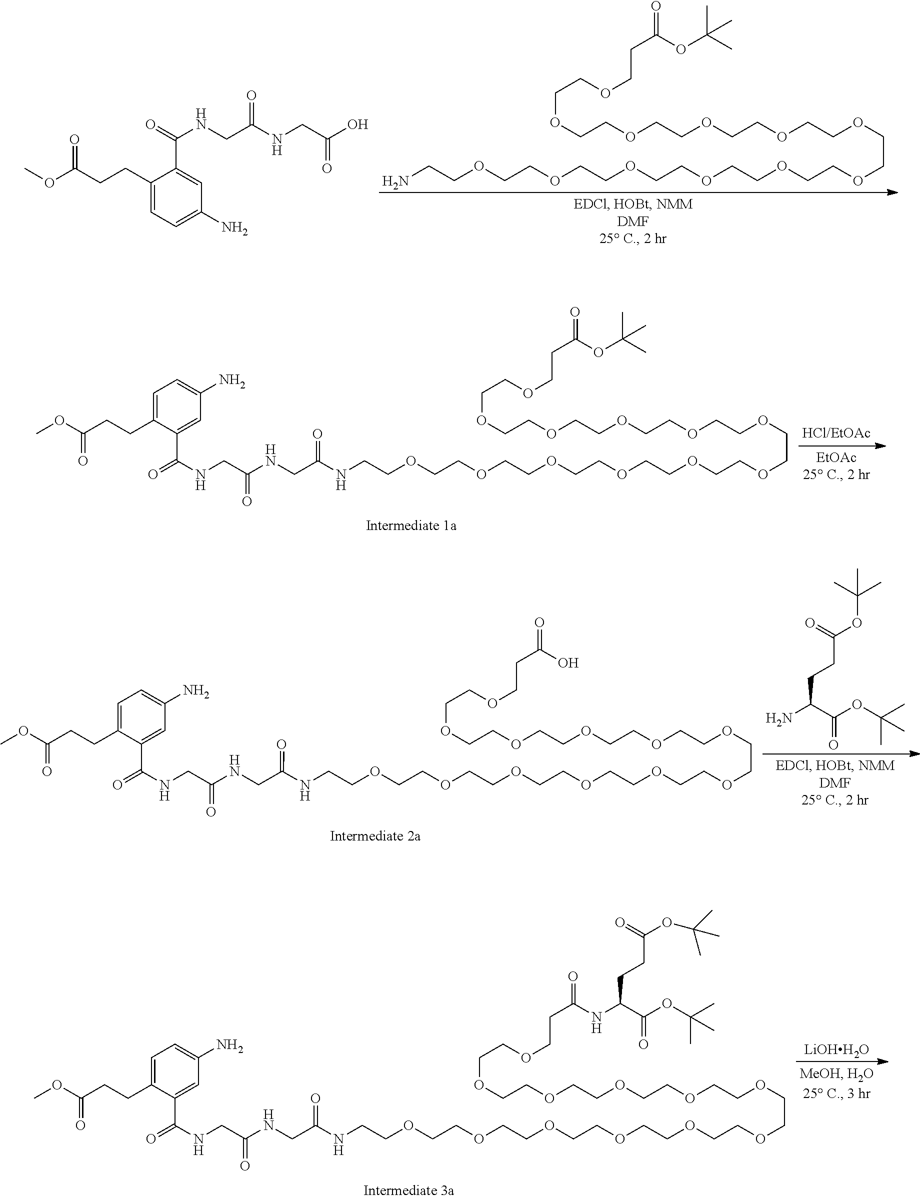

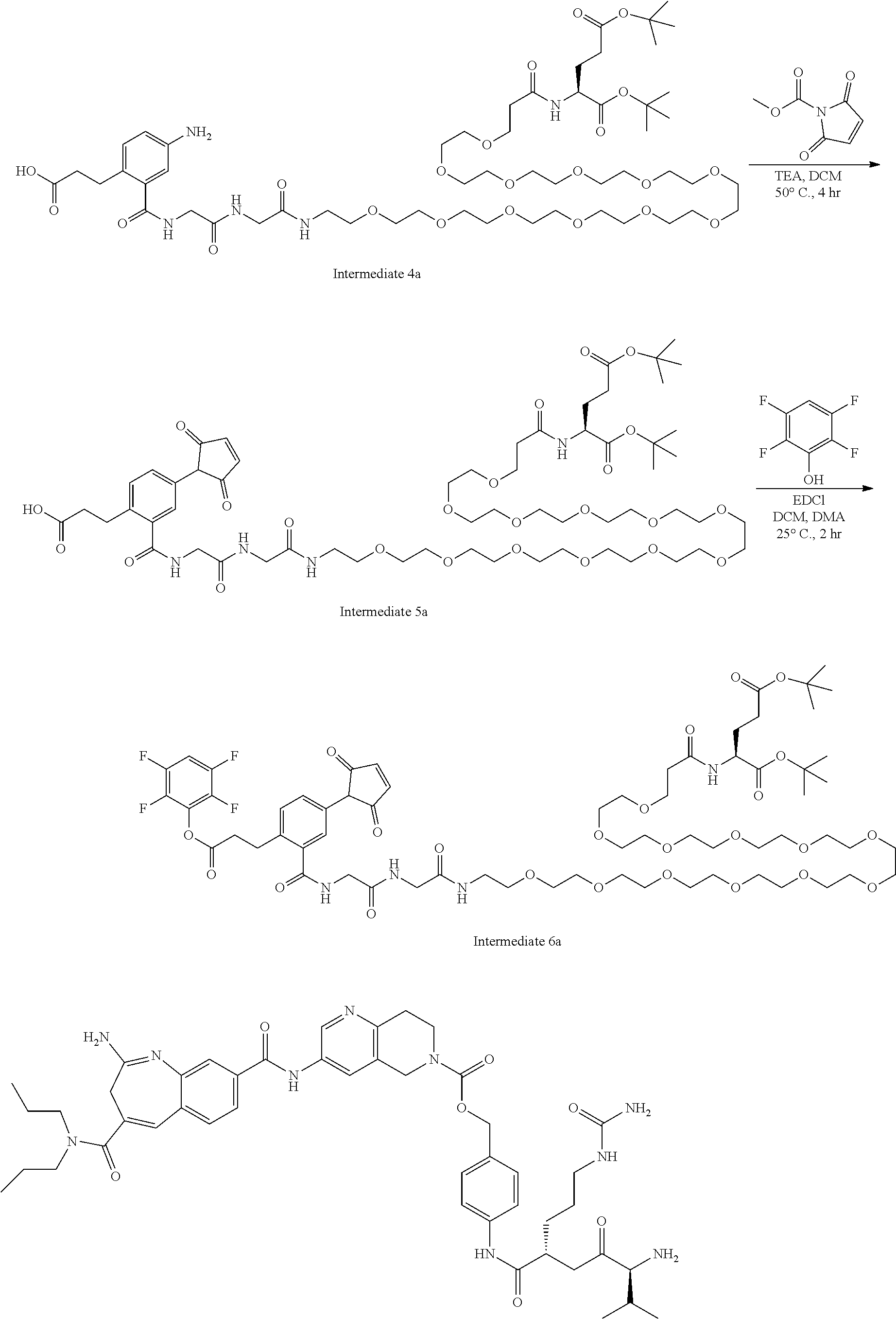

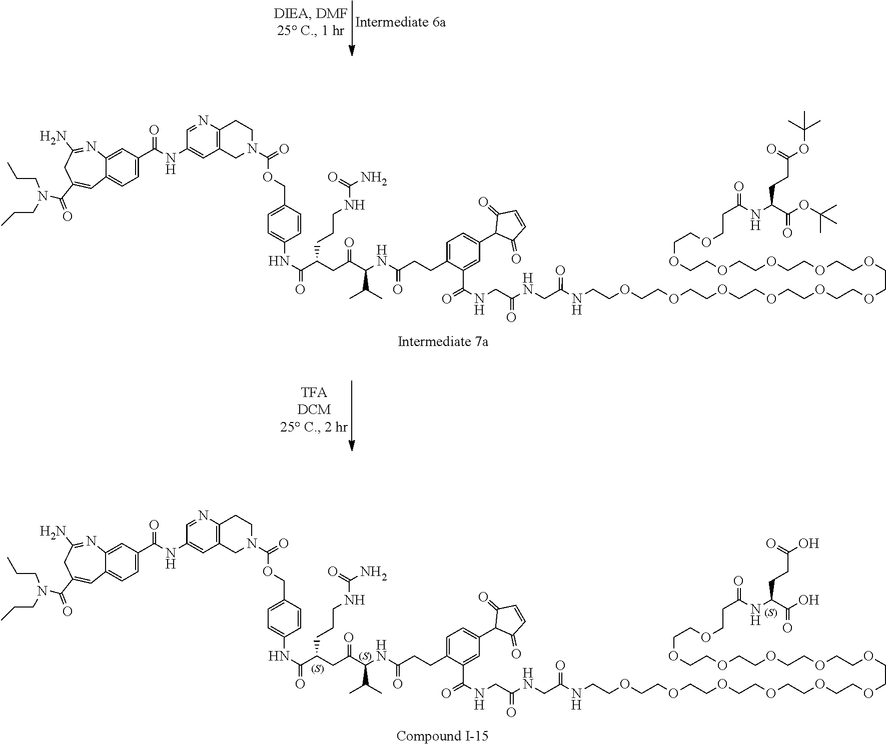

### Intermediate Example 1

### Preparation of tert-butyl 2-[[2-(benzyloxycarbonylamino)acetyl]amino]acetate

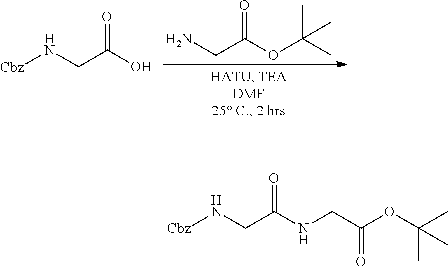

To a solution of 2-(benzyloxycarbonylamino)acetic acid (5.50 g, 26.3 mmol, 1.0 eq) in DMF (100 mL) was added triethylamine (TEA; 7.98 g, 78.9 mmol, 11.0 mL, 3.0 eq), HATU (12.0 g, 31.5 mmol, 1.2 eq) and tert-butyl 2-aminoacetate (3.45 g, 26.3 mmol, 1.0 eq), and then stirred at 25° C. for 2 hours. The reaction mixture was quenched by addition H₂O (150 mL) at 0° C., and then extracted with ethyl acetate (100 mL×3). The combined organic layers were washed with H₂O (80 mL×3), then were washed with brine (100 mL), dried over Na₂SO₄, filtered and concentrated under reduced pressure to give a residue. The residue was purified by silica gel chromatography (column height: 250 mm, diameter: 100 mm, 100-200 mesh silica gel, Ethyl acetate/Methanol=1/1) to give tert-butyl 2-[[2-(benzyloxycarbonylamino)acetyl]amino]acetate (8 g, 24.82 mmol, 94.40% yield) was obtained as a yellow oil.

¹H NMR (CDCl₃, 400 MHz) δ7.40-7.31 (m, 5H), 5.14 (s, 2H), 3.98-3.89 (m, 4H), 1.47 (s, 9H)

### Intermediate Example 2

### Preparation of tert-butyl 2-[(2-aminoacetyl)amino]acetate

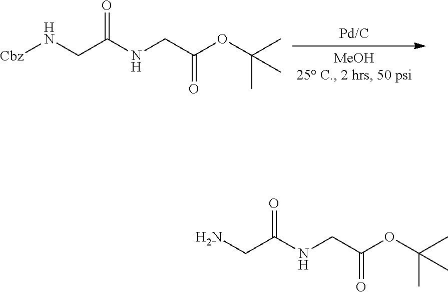 [SMILES: CC(C)(C)OC(=O)CNC(=O)CNC(=O)OCc1ccccc1 ; CC(C)(C)OC(=O)CNC(=O)CN]

To a solution of tert-butyl 2-[[2-(benzyloxycarbonylamino)acetyl]amino]acetate (8.80 g, 27.3 mmol, 1.0 eq) in methanol (150 mL) was added Pd/C (10%, 2.5 g) under N₂ atmosphere. The suspension was degassed and purged with H₂ for 3 times. The mixture was stirred under H₂ (50 Psi) at 25° C. for 2 hours. The reaction mixture was filtered and concentrated under reduced pressure to give a residue. Compound tert-butyl 2-[(2-aminoacetyl)amino]acetate (4.2 g, 22.31 mmol, 81.74% yield) was obtained as a white solid.

¹H NMR (CDCl₃, 400 MHz) δ 7.67 (s, 1H), 3.96 (d, J=5.6 Hz, 2H), 3.39 (s, 2H), 1.75 (s, 2H), 1.46 (s, 9H)

### Intermediate Example 3

### Preparation of methyl (E)-3-(4,4,5,5-tetramethyl-1,3,2-dioxaborolan-2-yl) prop-2-enoate

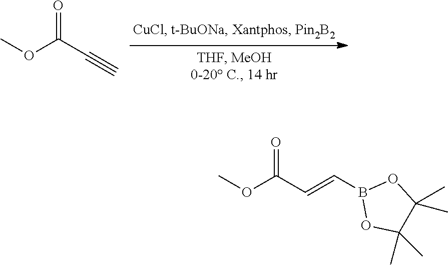

To a solution of CuCl (1.06 g, 10.7 mmol, 256 μL, 0.03 eq) and Xantphos (6.19 g, 10.7 mmol, 0.03 eq) in THF (450 mL) was added t-BuONa (2.06 g, 21.4 mmol, 0.06 eq) at 0° C. The reaction solution was stirred at 0° C. for 1 hour, followed by addition of a solution of Pin₂B₂ (90.6 g, 357 mmol, 1.0 eq) in THF (150 mL). The reaction solution was stirred under nitrogen atmosphere at 20° C. for one hour. Methyl prop-2-ynoate (30.0 g, 357 mmol, 29.7 mL, 1.0 eq) and methanol (22.9 g, 714 mmol, 28.9 mL, 2.0 eq) were added to the above reaction solution. And the reaction solution was stirred at 20° C. for another 12 hours. The result mixture was poured into ice-water (w/w=1/1) (300 mL) and stirred for 5 min. The aqueous phase was extracted with ethyl acetate (200 mL×3). The combined organic phase was washed with brine (50 mL×2), dried with anhydrous Na₂SO₄, filtered and concentrated in vacuo. The residue was purified by silica gel chromatography (column height: 250 mm, diameter: 100 mm, 100-200 mesh silica gel, Petroleum ether/Ethyl acetate=1/0, 3/1) to afford methyl (E)-3-(4,4,5,5-tetramethyl-1,3,2-dioxaborolan-2-yl) prop-2-enoate (70 g, 330 mmol, 92.51% yield) as colorless oil.

¹H NMR (400 MHz, CDCl₃) δ 6.83-6.75 (m, 1H), 6.68-6.59 (m, 1H), 3.77 (s, 3H), 1.29 (s, 12H).

### Intermediate Example 4

### Preparation of tert-butyl 2-bromo-5-nitro-benzoate

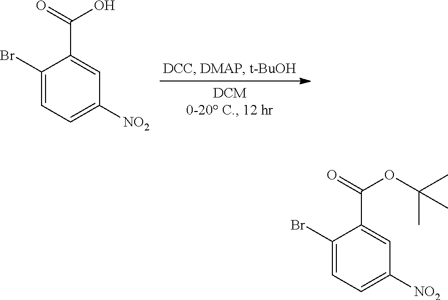 [SMILES: O=C(O)c1cc([N+](=O)[O-])ccc1Br ; CC(C)(C)OC(=O)c1cc([N+](=O)[O-])ccc1Br]

To a mixture of 2-bromo-5-nitro-benzoic acid (9.70 g, 39.4 mmol, 1.0 eq) in DCM (100 mL) was added DCC (8.95 g, 43.4 mmol, 8.77 mL, 1.1 eq) and DMAP (2.41 g, 19.7 mmol, 0.5 eq) at 0° C. The mixture was stirred at 0° C. for 10 min, then t-BuOH (4.38 g, 59.1 mmol, 5.66 mL, 1.5 eq) was added and stirred at 20° C. for 12 hours. The mixture was poured into ice-water (w/w=1/1) (50 mL) and stirred for 10 min. The aqueous phase was extracted with DCM (50 mL×2). The combined organic phase was washed with brine (10 mL×2), dried with anhydrous Na₂SO₄, filtered and concentrated in vacuo. The residue was purified by silica gel chromatography (column height: 250 mm, diameter: 100 mm, 100-200 mesh silica gel, Petroleum ether/Ethyl acetate=1/0, 5/1) to afford tert-butyl 2-bromo-5-nitro-benzoate (9.2 g, 30.5 mmol, 77.23% yield) as white solid.

¹H NMR (400 MHz, CDCl₃) δ 8.52 (d, J=2.8 Hz, 1H), 8.13 (dd, J=2.8, 8.8 Hz, 1H), 7.83 (d, J=8.8 Hz, 1H), 1.65 (s, 9H).

### Intermediate Example 5

### Preparation of tert-butyl 2-[(E)-3-methoxy-3-oxo-prop-1-enyl]-5-nitro-benzoate

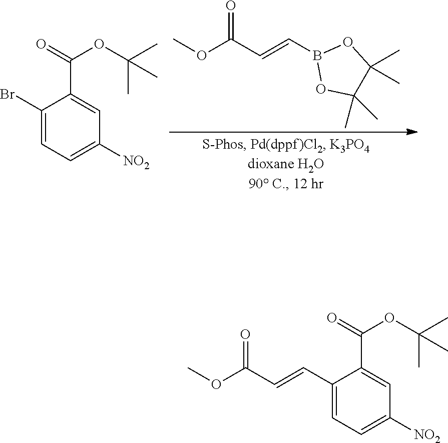

A mixture of methyl (E)-3-(4,4,5,5-tetramethyl-1,3,2-dioxaborolan-2-yl)prop-2-enoate (11.2 g, 53.0 mmol, 2.5 eq), tert-butyl 2-bromo-5-nitro-benzoate (6.40 g, 21.2 mmol, 1.0 eq), K₃PO₄ (6.74 g, 31.8 mmol, 1.5 eq), s-Phos (870 mg, 2.12 mmol, 0.1 eq), Pd₂(dba)₃ (970 mg, 1.06 mmol, 0.05 eq) in dioxane (120 mL) and H₂O (25 mL) was degassed and purged with N₂ for 3 times, and then the mixture was stirred at 90° C. for 12 hours under N₂ atmosphere. The mixture was poured into ice-water (w/w=1/1) (30 mL) and stirred for 10 min. The aqueous phase was extracted with ethyl acetate (50 mL×3). The combined organic phase was washed with brine (10 mL×3), dried with anhydrous Na₂SO₄, filtered and concentrated in vacuo. The residue was purified by silica gel chromatography (column height: 250 mm, diameter: 100 mm, 100-200 mesh silica gel, Petroleum ether/Ethyl acetate=1/0, 3/1) Afford tert-butyl 2-[(E)-3-methoxy-3-oxo-prop-1-enyl]-5-nitro-benzoate (6 g, 19.5 mmol, 92.17% yield) as yellow solid.

¹H NMR (400 MHz, CDCl₃) δ 8.74 (d, J=2.4 Hz, 1H), 8.41 (d, J=15.6 Hz, 1H), 8.34 (dd, J=2.4, 8.4 Hz, 1H), 7.72 (d, J=8.4 Hz, 1H), 6.38 (d, J=15.6 Hz, 1H), 3.85 (s, 3H), 1.65 (s, 9H).

### Intermediate Example 6

### Preparation of tert-butyl 5-amino-2-(3-methoxy-3-oxo-propyl)benzoate

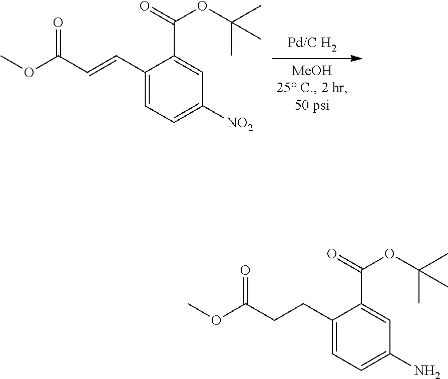 [SMILES: COC(=O)/C=C/c1ccc([N+](=O)[O-])cc1C(=O)OC(C)(C)C ; COC(=O)CCc1ccc(N)cc1C(=O)OC(C)(C)C]

A mixture of tert-butyl 2-[(E)-3-methoxy-3-oxo-prop-1-enyl]-5-nitro-benzoate (4.00 g, 13.0 mmol, 1.0 eq), Pd/C (400 mg, 13.0 mmol, 10% purity, 1.0 eq) in methanol (50 mL) was degassed and purged with H₂ (26.2 mg, 13.0 mmol) for 3 times, and then the mixture was stirred at 25° C. for 4 hours under H₂ atmosphere. The mixture was filtered and concentrated in vacuo to afford tert-butyl 5-amino-2-(3-methoxy-3-oxo-propyl)benzoate (3.50 g, 12.5 mmol, 96.26% yield) as yellow oil.

¹H NMR (400 MHz, CDCl₃) δ 7.14 (d, J=2.4 Hz, 1H), 7.04 (d, J=8.0 Hz, 1H), 6.73 (dd, J=2.4, 8.0 Hz, 1H), 3.67 (s, 3H), 3.12 (t, J=8.0 Hz, 2H), 2.61 (t, J=8.0 Hz, 2H), 1.59 (s, 9H).

### Intermediate Example 7

### Preparation of 5-amino-2-(3-methoxy-3-oxo-propyl)benzoic acid

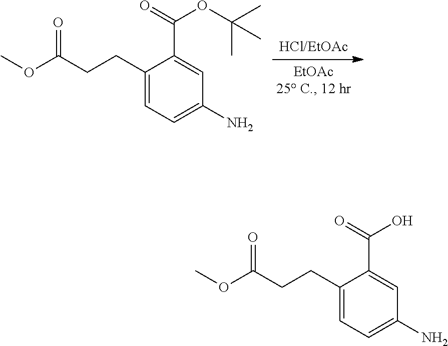

To a solution of tert-butyl 5-amino-2-(3-methoxy-3-oxo-propyl)benzoate (3.50 g, 12.5 mmol, 1.0 eq) in ethyl acetate (10 mL) was added HCl/ethyl acetate (4 M, 50 mL, 16.0 eq), and then stirred at 25° C. for 12 hours. The mixture was concentrated in vacuo to afford 5-amino-2-(3-methoxy-3-oxo-propyl)benzoic acid (3.20 g, 12.3 mmol, 98.35% yield, HCl) as white solid.

¹H NMR (400 MHz, DMSO-d₆) δ 7.69 (d, J=2.0 Hz, 1H), 7.39-7.31 (m, 2H), 3.57 (s, 3H), 3.15 (t, J=7.6 Hz, 2H), 2.59 (t, J=7.6 Hz, 2H).

### Intermediate Example 8

### Preparation of methyl 3-[4-amino-2-[[2-[(2-tert-butoxy-2-oxo-ethyl)amino]-2-oxo-ethyl]carbamoyl]phenyl]propanoate

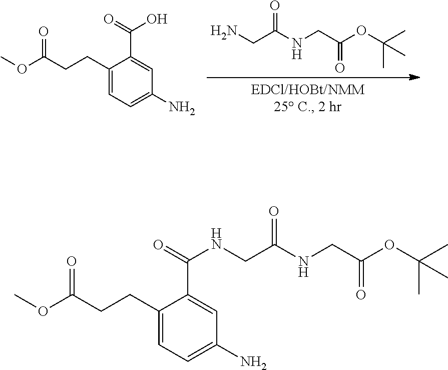 [SMILES: COC(=O)CCc1ccc(N)cc1C(=O)O ; CC(C)(C)OC(=O)CNC(=O)CN ; COC(=O)CCc1ccc(N)cc1C(=O)NCC(=O)NCC(=O)OC(C)(C)C]

To a solution of 5-amino-2-(3-methoxy-3-oxo-propyl)benzoic acid (3.20 g, 12.3 mmol, 1.0 eq, HCl) in DMF (40 mL) was added NMM (3.74 g, 37.0 mmol, 4.06 mL, 3.0 eq), HOBt (833 mg, 6.16 mmol, 0.5 eq), EDCI (4.72 g, 24.7 mmol, 2.0 eq) and tert-butyl 2-[(2-aminoacetyl)amino]acetate (2.78 g, 14.8 mmol, 1.2 eq) at 0° C., and then stirred at 20° C. for 1 hour. The mixture was poured into ice-water (w/w=1/1) (40 mL) and stirred for 10 min. The aqueous phase was extracted with ethyl acetate (50 mL×3). The combined organic phase was washed with brine (20 mL×2), dried with anhydrous Na₂SO₄, filtered and concentrated in vacuo. The residue was purified by silica gel chromatography (column height: 250 mm, diameter: 100 mm, 100-200 mesh silica gel, Petroleum ether/Ethyl acetate=1/0, 0/1) to afford methyl 3-[4-amino-2-[[2-[(2-tert-butoxy-2-oxo-ethyl)amino]-2-oxo-ethyl]carbamoyl]phenyl]propanoate (3.8 g, 9.66 mmol, 78.38% yield) as yellow oil.

¹H NMR (400 MHz, CDCl₃) δ 7.04 (d, J=8.0 Hz, 2H), 6.79-6.66 (m, 3H), 4.15 (d, J=5.6 Hz, 2H), 3.97 (d, J=5.2 Hz, 2H), 3.61 (s, 3H), 2.99 (t, J=7.2 Hz, 2H), 2.68 (t, J=7.2 Hz, 2H), 1.47 (s, 9H).

### Intermediate Example 9

### Preparation of 2-[[2-[[5-amino-2-(3-methoxy-3-oxo-propyl)benzoyl]amino] acetyl]amino]acetic acid

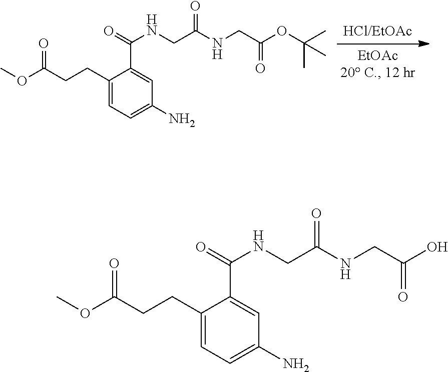

To a solution of methyl 3-[4-amino-2-[[2-[(2-tert-butoxy-2-oxo-ethyl)amino]-2-oxo-ethyl] carbamoyl]phenyl]propanoate (0.60 g, 1.53 mmol, 1.0 eq) in ethyl acetate (5 mL) was added HCl/ethyl acetate (4 M, 10 mL, 26.2 eq), and then stirred at 20° C. for 12 hours. The mixture was concentrated in vacuo to afford 2-[[2-[[5-amino-2-(3-methoxy-3-oxo-propyl)benzoyl]amino]acetyl]amino]acetic acid (550 mg, 1.47 mmol, 96.48% yield, HCl) as white solid.

¹H NMR (400 MHz, DMSO-d₆) δ 8.57 (t, J=6.0 Hz, 1H), 8.25 (t, J=6.4 Hz, 1H), 7.28 (d, J=8.0 Hz, 1H), 7.20-7.10 (m, 2H), 3.89 (d, J=6.0 Hz, 2H), 3.81 (d, J=6.0 Hz, 2H), 3.57 (s, 3H), 2.90 (t, J=8.0 Hz, 2H), 2.60 (t, J=8.0 Hz, 2H).

### Intermediate Example 10

### Preparation of Intermediate 1A

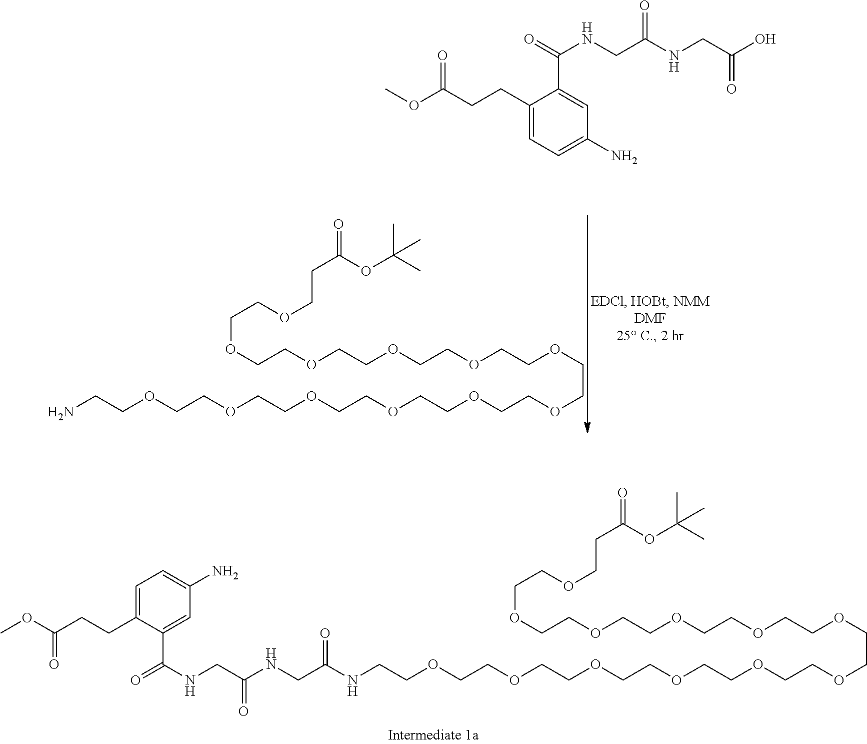 [SMILES: COC(=O)CCc1ccc(N)cc1C(=O)NCC(=O)NCC(=O)O ; CC(C)(C)OC(=O)CCOCCOCCOCCOCCOCCOCCOCCOCCOCCOCCOCCOCCN ; COC(=O)CCc1ccc(N)cc1C(=O)NCC(=O)NCC(=O)NCCOCCOCCOCCOCCOCCOCCOCCOCCOCCOCCOCCOCCC(=O)OC(C)(C)C]

A mixture of 2-[[2-[[5-amino-2-(3-methoxy-3-oxo-propyl)benzoyl]amino]acetyl] amino] acetic acid (320 mg, 856 μmol, 1.0 eq, HCl), tert-butyl 3-[2-[2-[2-[2-[2-[2-[2-[2-[2-[2-[2-(2-aminoethoxy)ethoxy]ethoxy]ethoxy]ethoxy]ethoxy]ethoxy]ethoxy]ethoxy]ethoxy]ethoxy]ethoxy]propanoate (576 mg, 856 μmol, 1.0 eq), EDCI (328 mg, 1.71 mmol, 2.0 eq), HOBt (57.8 mg, 428 μmol, 0.5 eq) and 4-methylmorpholine (259 mg, 2.57 mmol, 282 μL, 3.0 eq) in DMF (5 mL) was stirred at 25° C. for 2 hours. The reaction mixture was quenched by addition H₂O (10 mL) at 0° C., and then extracted with DCM/i-PrOH (v:v=3:1, 10 mL×5). The combined organic layers were concentrated under reduced pressure to give a residue. Desired product (950 mg, crude) was obtained as yellow oil. [M+H]⁺ (ESI): 994.10.

### Intermediate Example 11

### Preparation of Intermediate 2A

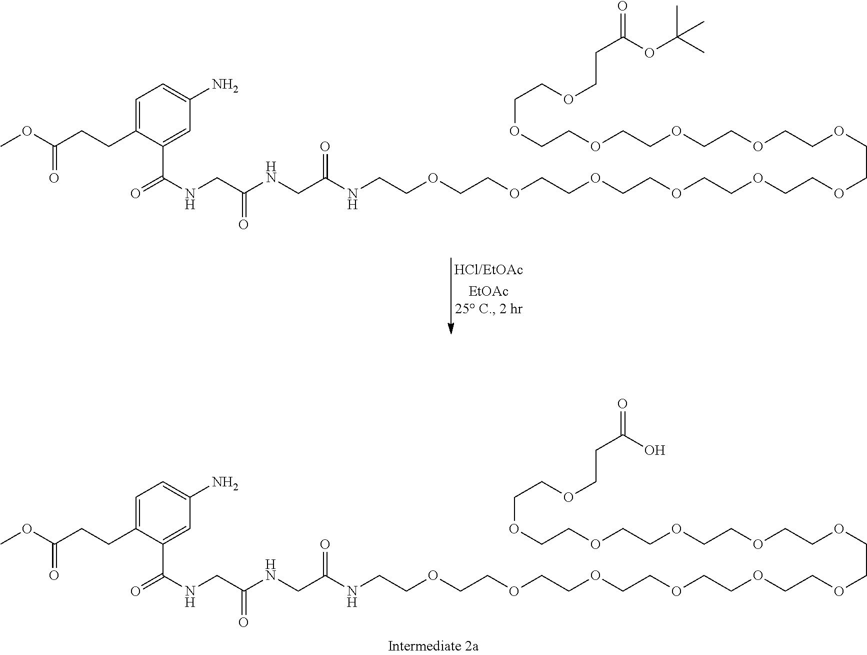 [SMILES: COC(=O)CCc1ccc(N)cc1C(=O)NCC(=O)NCC(=O)NCCOCCOCCOCCOCCOCCOCCOCCOCCOCCOCCOCCOCCC(=O)OC(C)(C)C ; COC(=O)CCc1ccc(N)cc1C(=O)NCC(=O)NCC(=O)NCCOCCOCCOCCOCCOCCOCCOCCOCCOCCOCCOCCOCCC(=O)O]

To a solution of Intermediate 1a (950 mg, 956 μmol, 1.0 eq) in ethyl acetate (5 mL) was added HCl/ethyl acetate (15 mL, 4M). The mixture was stirred at 25° C. for 2 hours. The reaction mixture was concentrated under reduced pressure. Desired product (890 mg, crude) was obtained as a yellow solid.

In another reaction, to a solution of Intermediate 1a (850 mg, 956 μmol, 1.0 eq.) in dioxane (5.0 mL) was added hydrochloride (gas)/ethyl acetate (15 mL, 4 mol/L). The mixture was stirred at 25° C. for 2 hours. The reaction mixture was concentrated under reduced pressure. Intermediate 2a (800 mg, crude) was obtained as a yellow solid. [M+H]⁺ (ESI): 936.20.

### Intermediate Example 12

### Preparation of Intermediate 3A

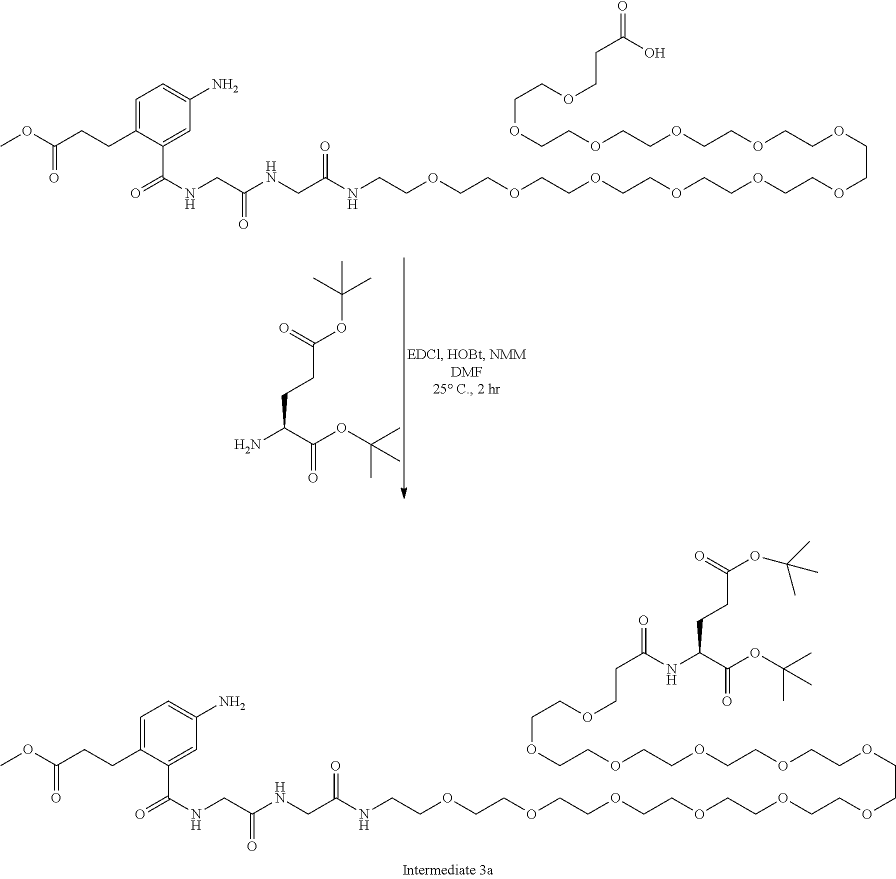 [SMILES: COC(=O)CCc1ccc(N)cc1C(=O)NCC(=O)NCC(=O)NCCOCCOCCOCCOCCOCCOCCOCCOCCOCCOCCOCCOCCC(=O)O ; CC(C)(C)OC(=O)CC[C@H](N)C(=O)OC(C)(C)C ; COC(=O)CCc1ccc(N)cc1C(=O)NCC(=O)NCC(=O)NCCOCCOCCOCCOCCOCCOCCOCCOCCOCCOCCOCCOCCC(=O)N[C@@H](CCC(=O)OC(C)(C)C)C(=O)OC(C)(C)C]

A mixture of Intermediate 2 (880 mg, 903 μmol, 1.0 eq, HCl), ditert-butyl (2S)-2-aminopentanedioate (588 mg, 1.99 mmol, 2.2 eq, HCl), EDCI (346 mg, 1.81 mmol, 2.0 eq), HOBt (61.0 mg, 451 μmol, 0.5 eq) and 4-methylmorpholine (274 mg, 2.71 mmol, 298 μL, 3.0 eq) in DMF (10 mL) was stirred at 25° C. for 2 hours. The reaction mixture was quenched by addition H₂O (20 mL) at 0° C., and then extracted with DCM/i-PrOH (v:v=3:1, 20 mL×5). The combined organic layers were concentrated under reduced pressure to give a residue. Desired product (900 mg, 763 μmol, 84.49% yield) was obtained as yellow oil.

In another reaction, a mixture of Intermediate 2a (800 mg, 0.903 mmol, 1.0 eq.), ditert-butyl (2S)-2-aminopentanedioate (588 mg, 1.99 mmol, 2.2 eq., HCl), 1-ethyl-3(3-dimethylpropylamine) carbodiimide (346 mg, 1.81 mmol, 2.0 eq.), 1-hydroxybenzotriazole (61.0 mg, 0.451 mmol, 0.5 eq.) and 4-methylmorpholine (274 mg, 2.71 mmol, 3.0 eq.) in N,N-dimethylformamide (10.0 mL) was stirred at 25° C. for 2 hours. The reaction mixture was quenched by addition water (20 mL) at 0° C., and then extracted with methylene chloride/isopropyl alcohol (v:v=3:1, 50 mL×5). The combined organic layers were concentrated under reduced pressure to give a residue. Column, C₁₈ silica gel; mobile phase, acetonitrile in water (0.1% trifluoroacetic acid/formic acid), 10% to 70% gradient in 20 min; detector, UV 254 nm. 900 mg (89%) of Intermediate 3a was obtained as yellow oil. [M+H]⁺ (ESI): 1179.05

### Intermediate Example 13

### Preparation of Intermediate 4A

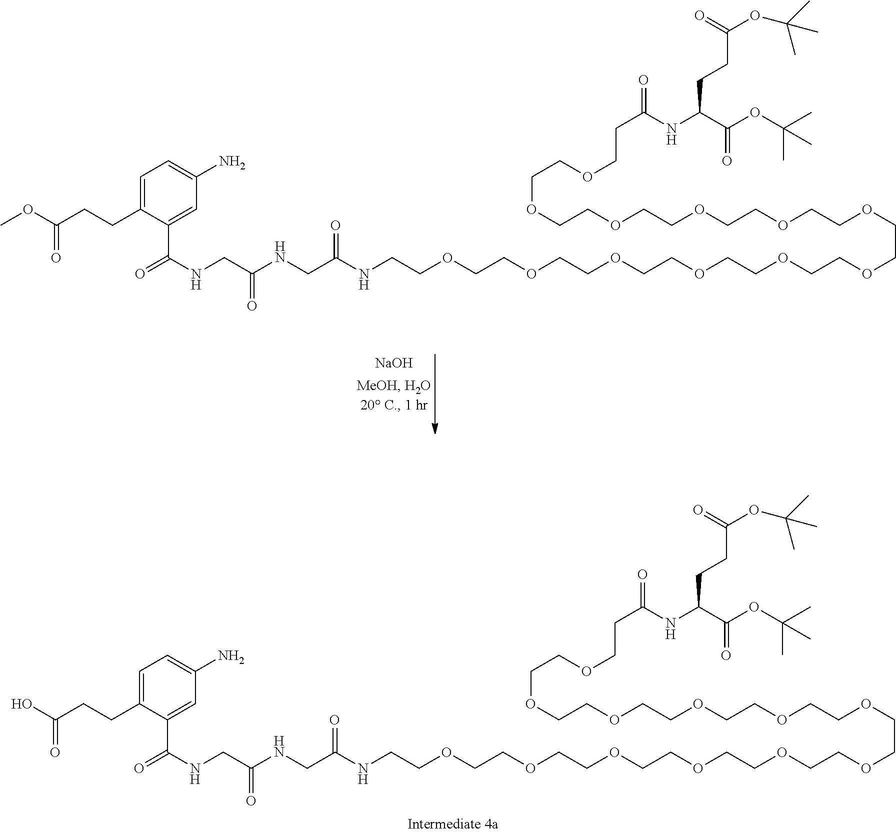 [SMILES: COC(=O)CCc1ccc(N)cc1C(=O)NCC(=O)NCC(=O)NCCOCCOCCOCCOCCOCCOCCOCCOCCOCCOCCOCCOCCC(=O)N[C@@H](CCC(=O)OC(C)(C)C)C(=O)OC(C)(C)C ; "NaOH" ; CC(C)(C)OC(=O)CC[C@H](NC(=O)CCOCCOCCOCCOCCOCCOCCOCCOCCOCCOCCOCCOCCNC(=O)CNC(=O)CNC(=O)c1cc(N)ccc1CCC(=O)O)C(=O)OC(C)(C)C]

To a solution of Intermediate 3a (800 mg, 679 μmol, 1.0 eq) in methanol (2.5 mL) and H₂O (2.5 mL) was added NaOH (136 mg, 3.39 mmol, 5.0 eq), and then stirred at 20° C. for 1 hour. The mixture was concentrated in vacuo to remove methanol, and then diluted with H₂O (20 mL), the pH of the aqueous phase was adjusted to ˜7 with HCl (2M), and extracted with DCM/i-PrOH (v:v=3:1, 15 mL×5). The combined organic layers were dried over Na₂SO₄, filtered and concentrated under reduced pressure to give a residue to afford desired product (700 mg, crude) as red oil.

In another reaction, to a solution of Intermediate 3a (800 mg, 0.68 mmol, 1.0 eq.) in methanol (8.0 mL) and water (2.5 mL) was added sodium hydroxide (136 mg, 3.39 mmol, 5.0 eq.), and then stirred at 0° C. for 2 h. The mixture was concentrated in vacuum to remove methanol, and then diluted with water (20 mL), the pH of the aqueous phase was adjusted to ˜7 with hydrochloric acid (2 mol/L), and extracted with methylene chloride/isopropyl alcohol (v/v=3:1, 30 mL×5). The combined organic layers were dried over sodium sulfate, filtered and concentrated under reduced pressure to give a residue to afford Intermediate 4a (700 mg, crude) as red oil. [M+H]⁺ (ESI): 1165.10.

### Intermediate Example 14

### Preparation of Intermediate 5A

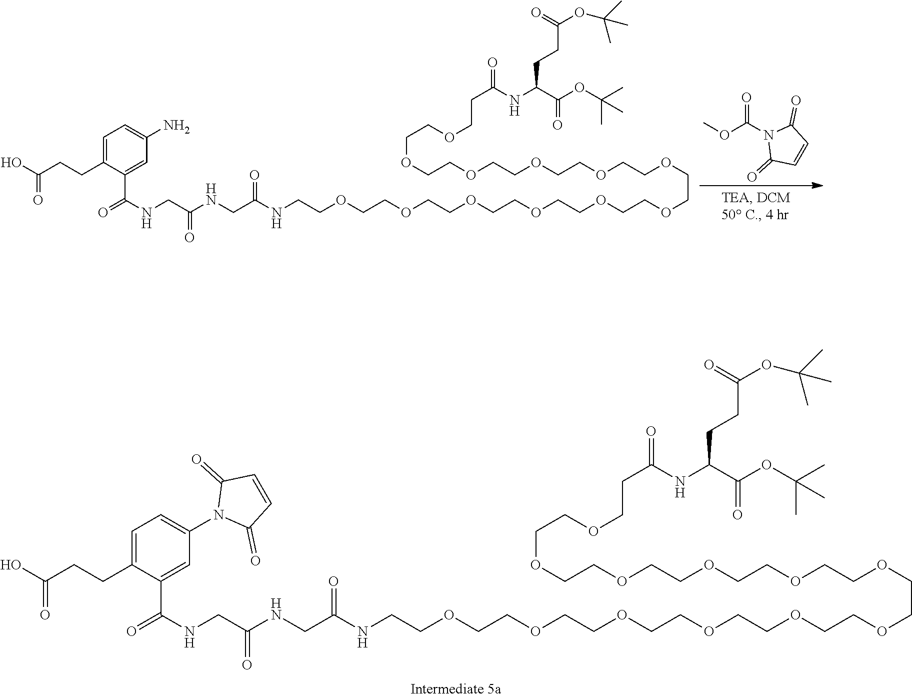 [SMILES: CC(C)(C)OC(=O)CC[C@H](NC(=O)CCOCCOCCOCCOCCOCCOCCOCCOCCOCCOCCOCCOCCNC(=O)CNC(=O)CNC(=O)c1cc(N)ccc1CCC(=O)O)C(=O)OC(C)(C)C ; COC(=O)N1C(=O)C=CC1=O ; CC(C)(C)OC(=O)CC[C@H](NC(=O)CCOCCOCCOCCOCCOCCOCCOCCOCCOCCOCCOCCOCCNC(=O)CNC(=O)CNC(=O)c1cc(N2C(=O)C=CC2=O)ccc1CCC(=O)O)C(=O)OC(C)(C)C]

To a solution of Intermediate 4a (250 mg, 214 μmol, 1.0 eq) and methyl 2,5-dioxopyrrole-1-carboxylate (49.9 mg, 322 μmol, 49.9 μL, 1.5 eq) in DCM (3 mL) was added TEA (65.1 mg, 644 μmol, 89.6 μL, 3.0 eq), and then stirred at 50° C. for 4 hours. The pH of the mixture was adjusted ˜6 with TFA, and diluted with addition H₂O (10 mL) and extracted with DCM:i-PrOH (v:v=3:1, 10 mL×5). The combined organic layers were concentrated under reduced pressure to give a residue. Desired product (275 mg, crude) was obtained as colorless oil.

In another reaction, to a solution of Intermediate 4a (700 mg, 0.60 mmol, 1.0 eq.) and methyl 2,5-dioxopyrrole-1-carboxylate (139.8 mg, 0.92 mmol, 1.5 eq.) in dichloromethane (7.0 mL) was added trimethylamine (182.2 mg, 1.80 mmol, 3.0 eq.), and then stirred at 50° C. for 4 h. The pH of the mixture was adjusted ˜6 with trifluoroacetic acid, and diluted with addition water (10 mL) and extracted with methylene chloride:isopropyl alcohol (v/v=3:1, 30 mL×5). The combined organic layers were concentrated under reduced pressure to give a residue. Column, C₁₈ silica gel; mobile phase, acetonitrile in water (0.1% trifluoroacetic acid/formic acid), 10% to 70% gradient in 20 min; detector, UV 254 nm. 306 mg (41%) of Intermediate 5a was obtained as colorless oil. [M+H]⁺ (ESI): 1245.32.

### Intermediate Example 15

### Preparation of Intermediate 6A

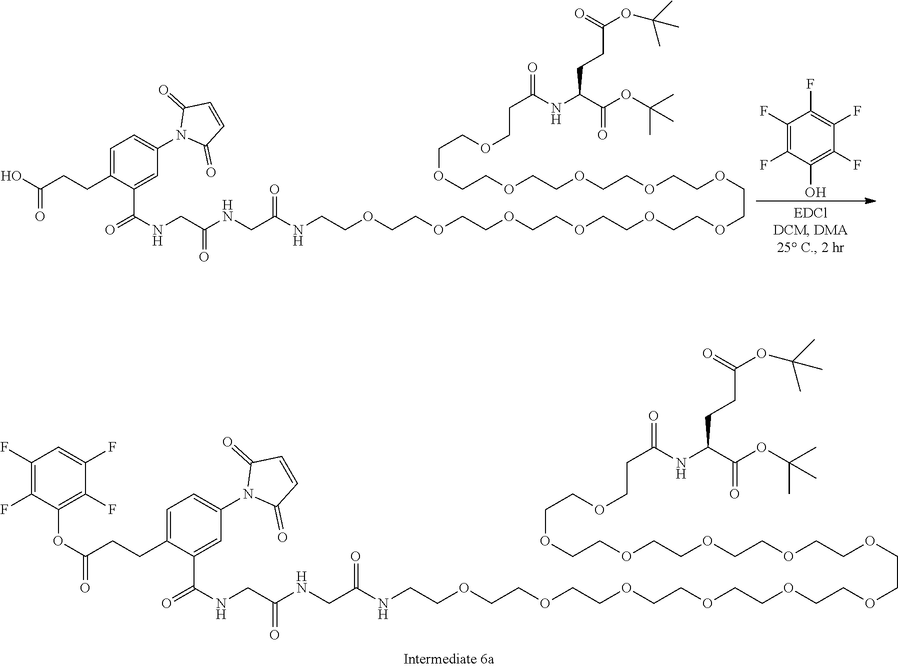

A mixture of Intermediate 5a (270 mg, 216 μmol, 1.0 eq) and EDCI (166 mg, 867 μmol, 4.0 eq) in DCM (2 mL) and DMA (2 mL) was stirred at 25° C. for 2 hours. The reaction mixture was concentrated under reduced pressure to remove DCM. The residue was purified by prep-HPLC (column: Phenomenex Luna 80·30 mm·3 μm; mobile phase: [water (TFA)-acetonitrile]; B %: 35%-70%, 8 min). The eluent was removed under freeze drying. Desired product (90 mg, 64.6 μmol, 29.79% yield) was obtained as colorless oil.

¹H NMR (DMSO-d₆, 400 MHz) δ 7.94-7.84 (m, 1H), 7.50 (d, J=8.4 Hz, 1H), 7.43-7.34 (m, 2H), 7.18 (s, 2H), 4.13 (dd, J=5.2, 9.1 Hz, 1H), 3.89 (s, 2H), 3.70 (s, 2H), 3.60-3.58 (m, 3H), 3.51-3.47 (m, 44H), 3.39 (t, J=6.0 Hz, 2H), 3.22-3.17 (m, 2H), 3.14 (s, 3H), 2.42-2.32 (m, 2H), 2.27-2.19 (m, 2H), 1.95-1.83 (m, 1H), 1.77-1.65 (m, 1H), 1.38 (s, 18H).

In another reaction, a mixture of 2,3,5,6-tetrafluorophenol (108 mg, 0.65 mmol, 3.0 eq.), Intermediate 5a (270 mg, 216 μmol, 1.0 eq.) and 1-ethyl-3(3-dimethylpropylamine) carbodiimide (166 mg, 867 μmol, 4.0 eq.) in N,N-dimethylformamide (3.0 mL) was stirred at 25° C. for 2 h. The reaction mixture was concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 90 mg (30%) of Intermediate 6a was obtained as colorless oil. [M+H]⁺ (ESI): 1394.45.

Additionally, Intermediate 6a can be deprotected according to the following reaction scheme and protocol:

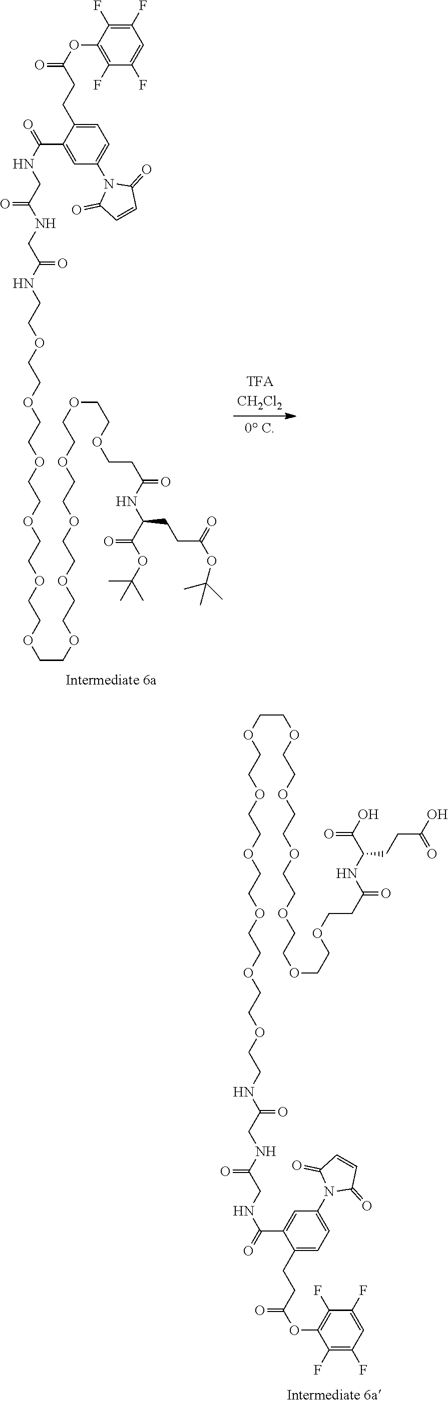 [SMILES: CC(C)(C)OC(=O)CC[C@H](NC(=O)CCOCCOCCOCCOCCOCCOCCOCCOCCOCCOCCOCCOCCNC(=O)CNC(=O)CNC(=O)c1cc(N2C(=O)C=CC2=O)ccc1CCC(=O)Oc1c(F)c(F)cc(F)c1F)C(=O)OC(C)(C)C ; O=C(O)CC[C@H](NC(=O)CCOCCOCCOCCOCCOCCOCCOCCOCCOCCOCCOCCOCCNC(=O)CNC(=O)CNC(=O)c1cc(N2C(=O)C=CC2=O)ccc1CCC(=O)Oc1c(F)c(F)cc(F)c1F)C(=O)O]

A solution of Intermediate 6a (90 mg, 0.07 mmol, 1.0 eq.) in dichloromethane (1.0 mL) with an inert atmosphere of nitrogen, was added trifluoroacetic acid (0.25 mL) at 0° C. The resulting solution was stirred for 4 h at 0° C. and concentrated under vacuum and concentrated under reduced pressure. 90 mg (crude) of Intermediate 6a′ was obtained as a yellow oil. [M+H]⁺ (ESI): 1279.95.

### Intermediate Example 16

### Preparation of Intermediate 7A

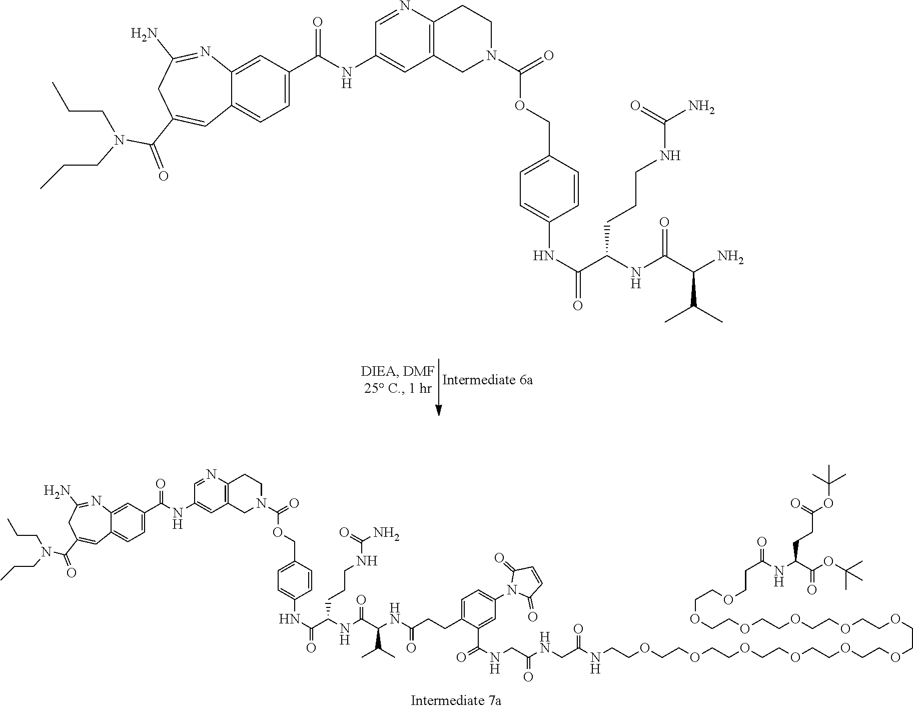

To a solution of [4-[[(2S)-2-[[(2S)-2-amino-3-methyl-butanoyl]amino]-5-ureido-pentanoyl]amino]phenyl]methyl 3-[[2-amino-4-(dipropylcarbamoyl)-3H-1-benzazepine-8-carbonyl]amino]-7,8-dihydro-5H-1,6-naphthyridine-6-carboxylate (20.0 mg, 23.0 μmol, 1.0 eq) in DMF (0.5 mL) was added diisopropylethylamine (DIEA) (8.95 mg, 69.2 μmol, 12.0 μL, 3.0 eq) and Intermediate 6a (32.1 mg, 23.0 μmol, 1.0 eq). The mixture was stirred at 25° C. for 1 hour. The pH of the mixture was adjusted to ˜6 with trifluoroacetic acid (TFA) at 0° C., and diluted with addition H₂O (5 mL) and extracted with dichloromethane (DCM):isopropyl alcohol (i-PrOH) (v:v=3:1, 5 mL×5). The combined organic layers were concentrated under reduced pressure to give a residue. Desired product (55 mg, crude) was obtained as yellow oil.

### Intermediate Example 17

### Synthesis of (19S,22R,23R,23aR,25R,27aR,29R,210R,210aR,212S,214aR,37R,E)-23,210-difluoro-25,212-dimercapto-23,23a,27a,29,210,210a,214,214a-octahydro-19H,22H,27H,37H-4,9-diaza-1(9,6)-purina-3(7,4)-pyrrolo[2,3-d]pyrimidina-2(2,9)-difuro[3,2-d:3′,2′-j][1,3,7,9]tetraoxa[2,8]diphosphacyclododecinacyclononaphan-6-ene 25,212-dioxide

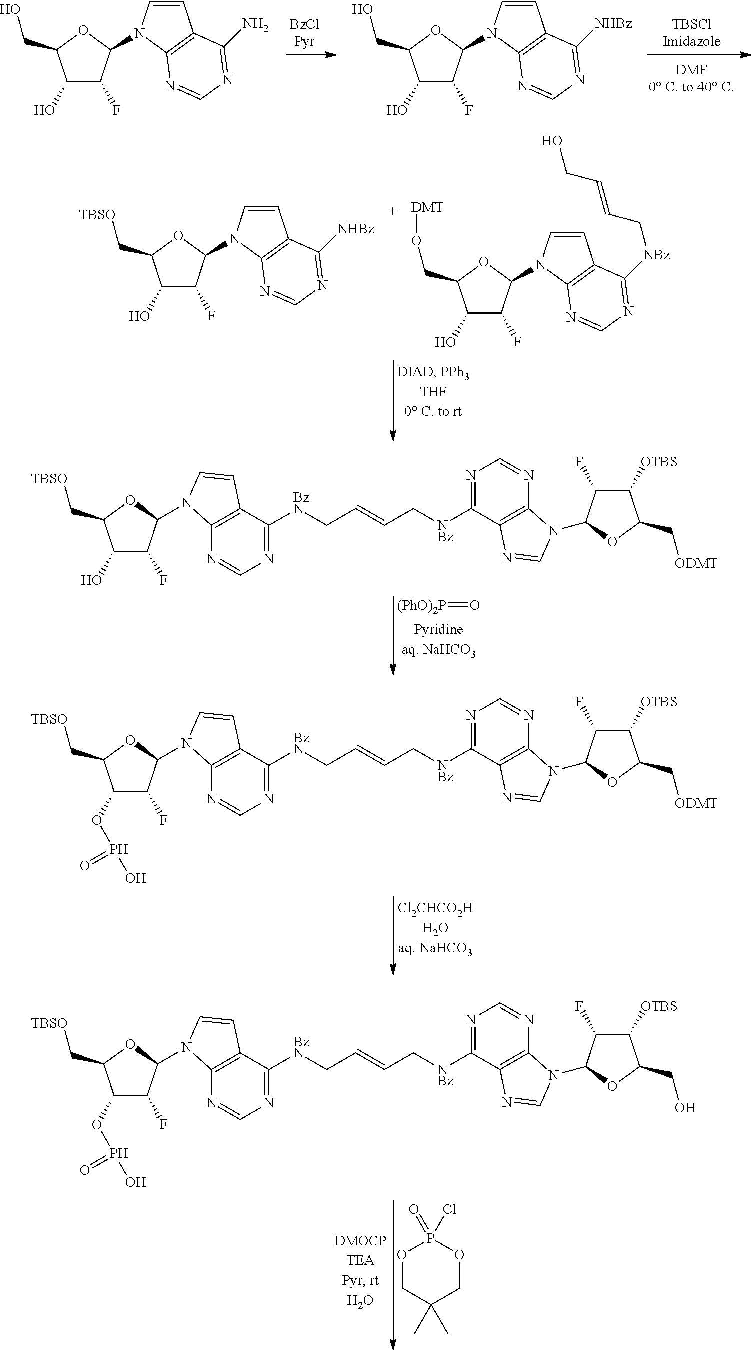

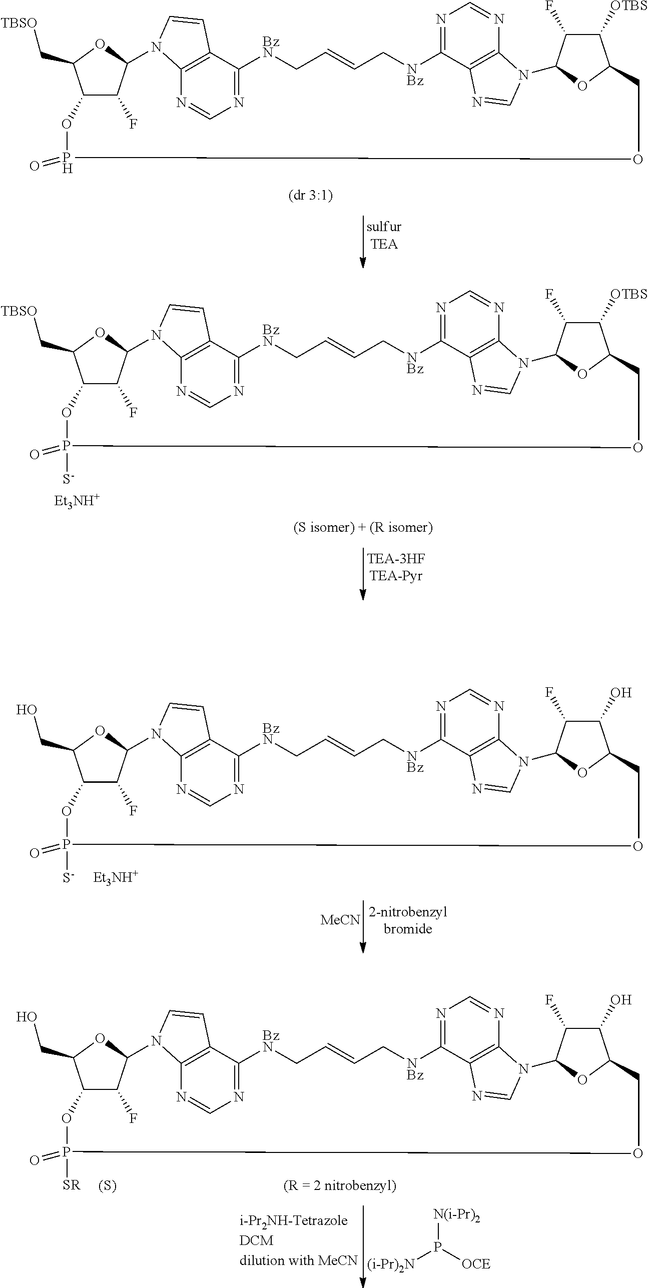

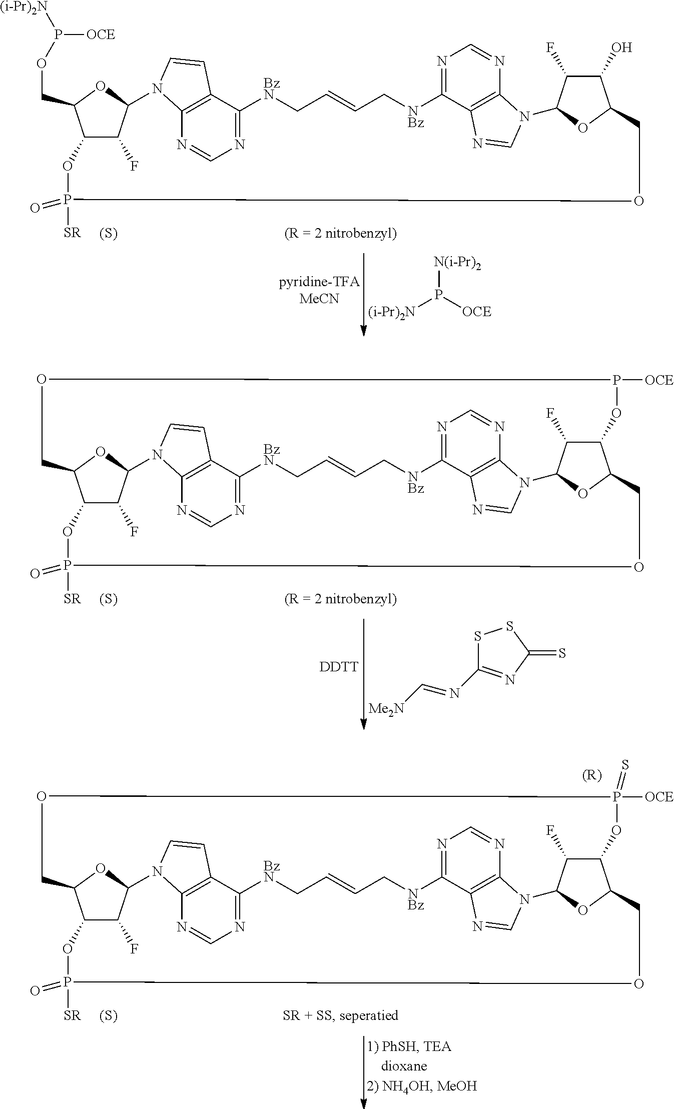

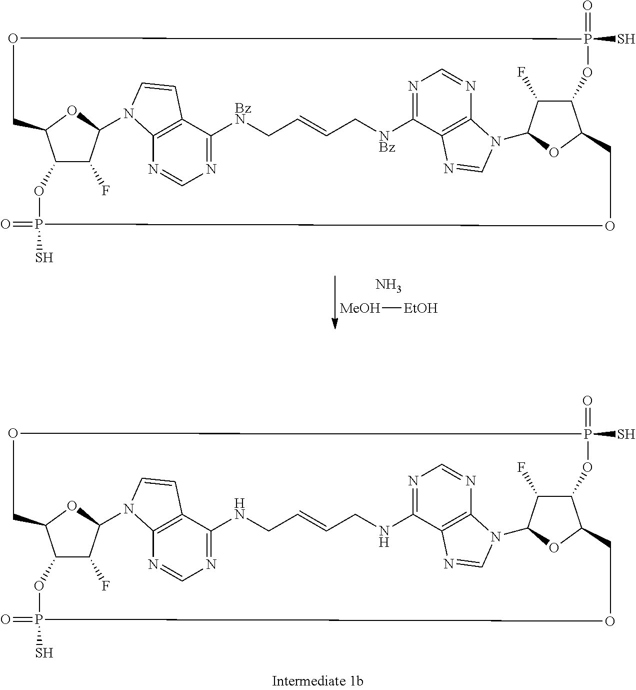

Intermediate 1b (alternatively in its ammonium salt form) is obtained using the reaction conditions and synthetic scheme shown above. Reaction methods, reagents, purification techniques, and the like, are known in the art. For example, certain aspects of method of preparing compounds of the present disclosure can be found in PCT Publication Nos. 2018/152453 and/or 2018/152450, which are hereby incorporated by reference in its entirety.

(2R,3R,4R,5R)-5-(4-amino-7H-pyrrolo[2,3-d]pyrimidin-7-yl)-4-fluoro-2-(hydroxymethyl)tetrahydrofuran-3-ol is protected using Bz-Cl and pyridine to obtain N-(7-((2R,3R,4R,5R)-3-fluoro-4-hydroxy-5-(hydroxymethyl)tetrahydrofuran-2-yl)-7H-pyrrolo[2,3-d]pyrimidin-4-yl)benzamide, which is then protected using TBS-Cl and imidazole in DMF and cooled to 0° C. before warming the reaction mixture to 40° C. The resultant product is then reacted with N-(9-((2R,3R,4R,5R)-5-((bis(4-methoxyphenyl)(phenyl)methoxy)methyl)-4-((tert-butyldimethylsilyl)oxy)-3-fluorotetrahydrofuran-2-yl)-9H-purin-6-yl)-N-((E)-4-hydroxybut-2-en-1-yl)benzamide using DIAD and triphenylphosphine in THF and cooling the reaction to 0° C. and allowing the reaction mixture to warm to room temperature over time. The free OH group of the resultant product is then converted to a phosphonate with reagents shown under basic conditions. The DMT protecting group of the resultant product is then removed with dichloroacetic acid, water and aqueous sodium bicarbonate. The de-protected product is then reacted and forms an intramolecular link with the phosphonate group as shown. The sulfur functional group is then added to the resultant product using sulfur and trimethylamine. The TBS protecting groups are then removed using the reagents as shown. The resultant product is then functionalized with a 2-nitrobenzyl group. The resultant product is then converted to the phosphoramidite in dichloromethane (diluted with acetonitrile). The phosphoramidite product then forms an intramolecular link via the free OH group as shown. The linked product is then converted to the sulfur containing product. The resultant product is reacted using benzenethiol, trimethylamine, and dioxane. The resultant product is then de-protected to afford the desired compound, which is used as the starting material in Reaction Scheme 2, below.

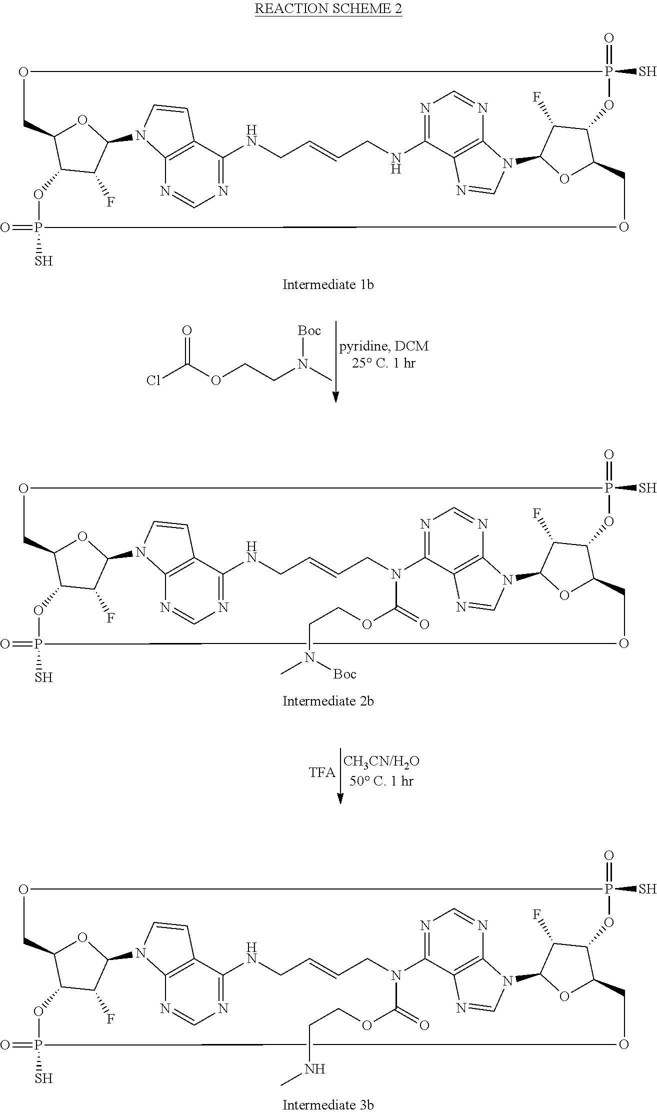 [SMILES: O=[P@]1(S)OC[C@H]2O[C@@H]3[C@H](F)[C@@H]2O[P@](=O)(S)OC[C@H]2O[C@H]([C@H](F)[C@@H]2O1)n1ccc2c(ncnc21)NC/C=C/CNc1ncnc2c1ncn23 ; CN(CCOC(=O)Cl)C(=O)OC(C)(C)C ; CN(CCOC(=O)N1C/C=C/CNc2ncnc3c2ccn3[C@@H]2O[C@@H]3CO[P@@](=O)(S)O[C@H]4[C@@H](F)[C@@H](O[C@@H]4CO[P@](=O)(S)O[C@H]3[C@H]2F)n2cnc3c1ncnc32)C(=O)OC(C)(C)C ; CNCCOC(=O)N1C/C=C/CNc2ncnc3c2ccn3[C@@H]2O[C@@H]3CO[P@@](=O)(S)O[C@H]4[C@@H](F)[C@@H](O[C@@H]4CO[P@](=O)(S)O[C@H]3[C@H]2F)n2cnc3c1ncnc32]

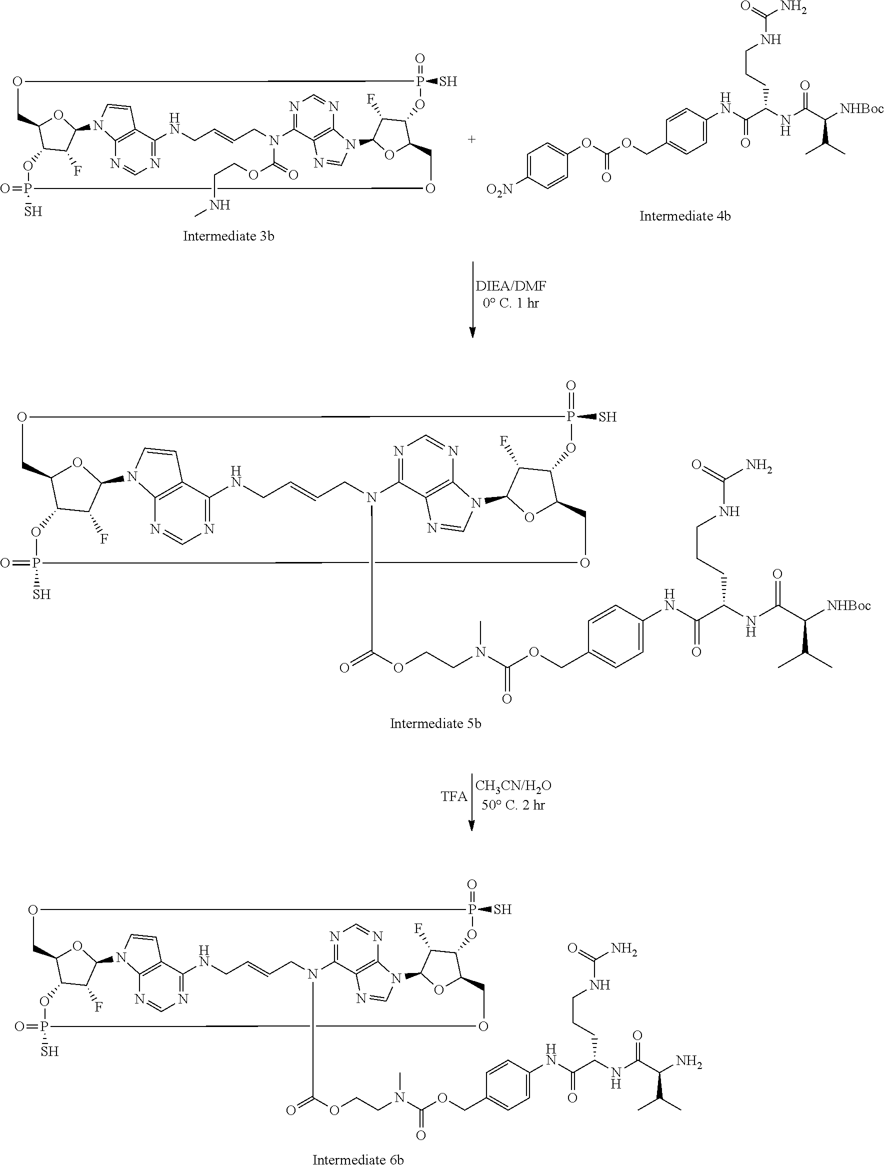

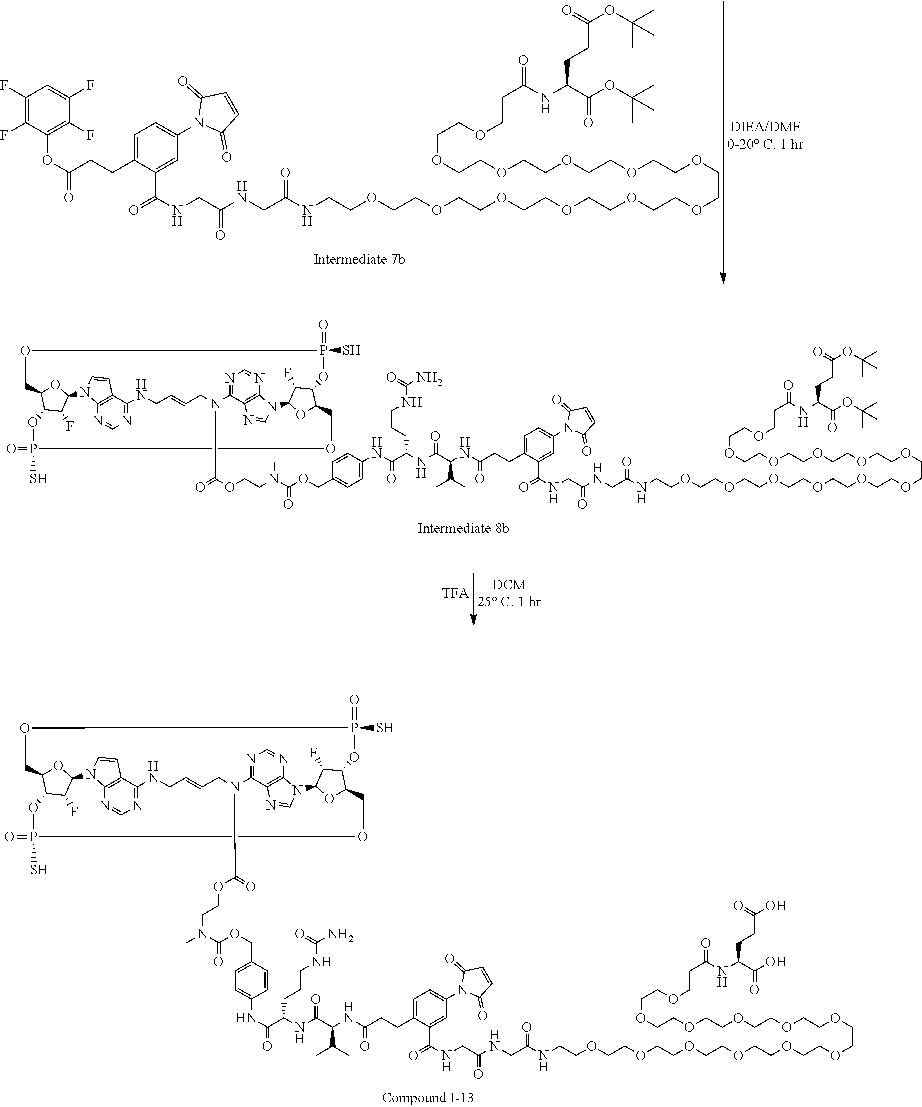

### Intermediate Example 18

### Preparation of Intermediate 2B

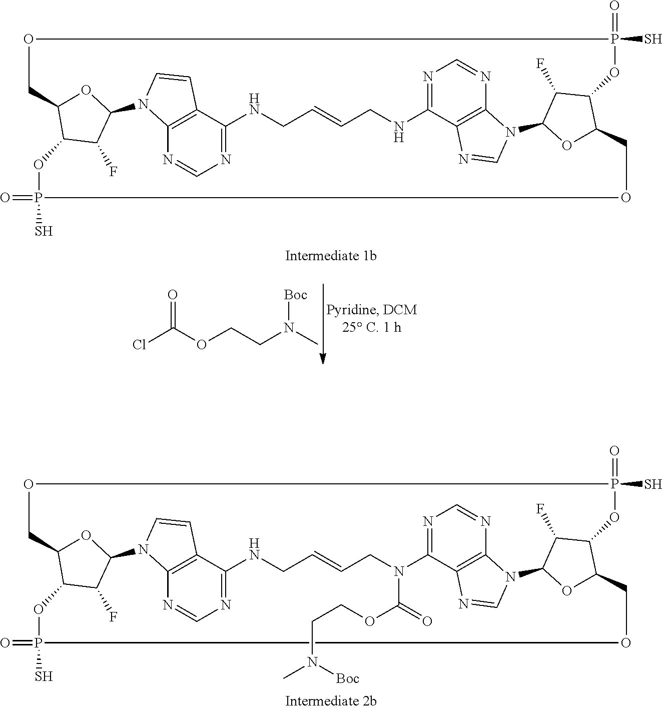

To a solution of Intermediate 1b (100 mg, 134 μmol, 1 eq) in DCM (6 mL) was added pyridine (318 mg, 4.02 mmol, 325 μL, 30 eq) and 2-[tert-butoxycarbonyl(methyl)amino]ethyl carbonochloridate (542 mg, 2.28 mmol, 17 eq). The mixture was stirred at 25° C. for 1 hr. The mixture was concentrated in vacuo. The residue was purified by prep-HPLC (column: C₁₈₋₄ 150·30 mm·5 μm; mobile phase: [A: 0.1 M water (TFA) B: acetonitrile]; B %: 15%-45%, 20 min) to afford desired product (50 mg, 47.7 μmol, 35.57% yield, TEA) as white solid.

### Intermediate Example 19

### Preparation of Intermediate 3B

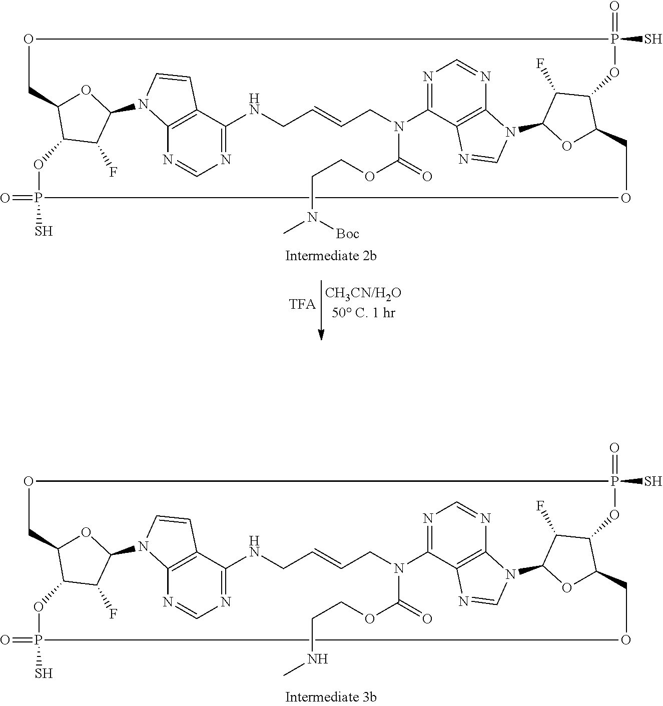

To a solution of Intermediate 2b (100 mg, 95.4 μmol, 1.0 eq, TEA) in acetonitrile (0.5 mL) and H₂O (0.5 mL) was added TFA (544 mg, 4.77 mmol, 353 μL, 50 eq). The mixture was stirred at 50° C. for 1 hr. The mixture was concentrated in vacuo to afford desired product (84 mg, 87.4 μmol, 91.63% yield, TFA) as white solid.

### Intermediate Example 20

### Preparation of Intermediate 5B

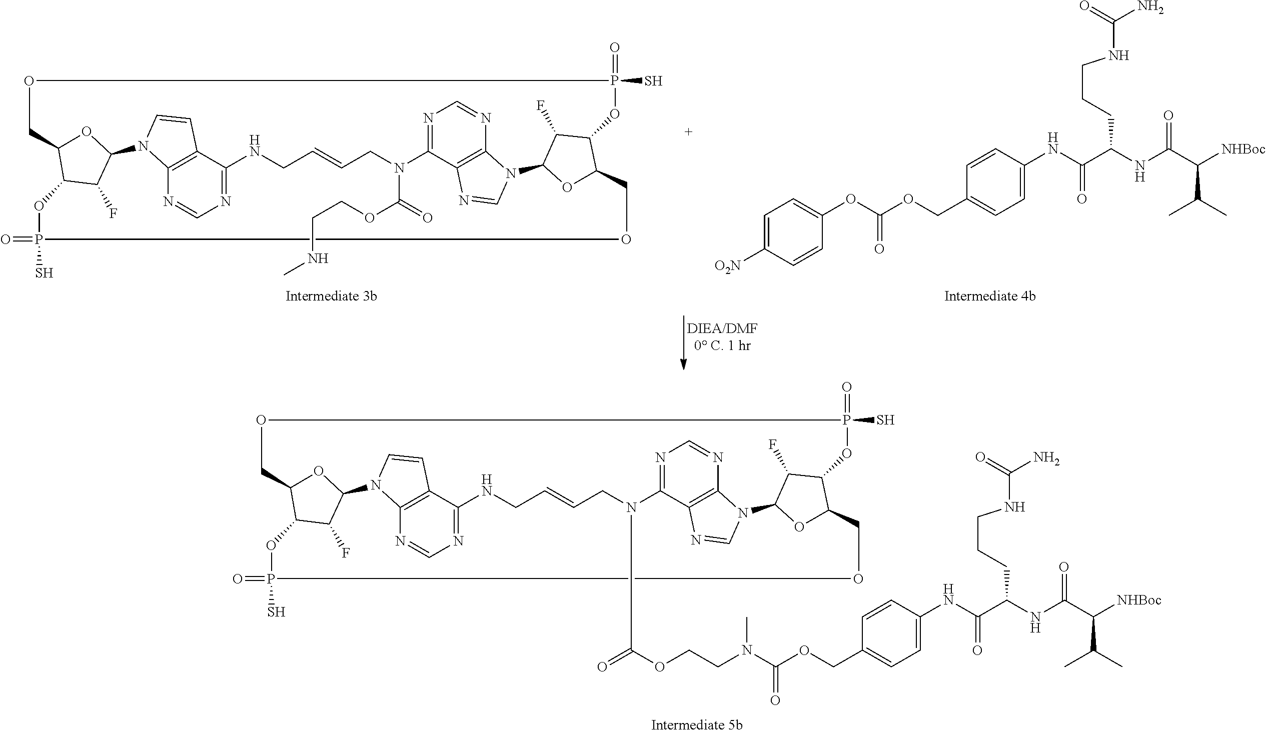

To a solution of Intermediate 3b (40 mg, 41.6 μmol, 1 eq, TFA) in DMF (0.2 mL) was added DIEA (16.1 mg, 125 μmol, 21.8 μL, 3.0 eq) and Intermediate 4b (40.3 mg, 62.5 μmol, 1.5 eq). The mixture was stirred at 0° C. for 1 hr. The pH of the mixture was adjusted to ˜6 with TFA, then concentrated in vacuo. The residue was diluted with H₂O (5 mL), the aqueous phase was extracted with ethyl acetate (5 mL) to remove byproduct. The water phase was freeze-drying to afford desired product (50 mg, 37.0 μmol, 88.81% yield) as white solid.

### Intermediate Example 21

### Preparation of Intermediate 6B

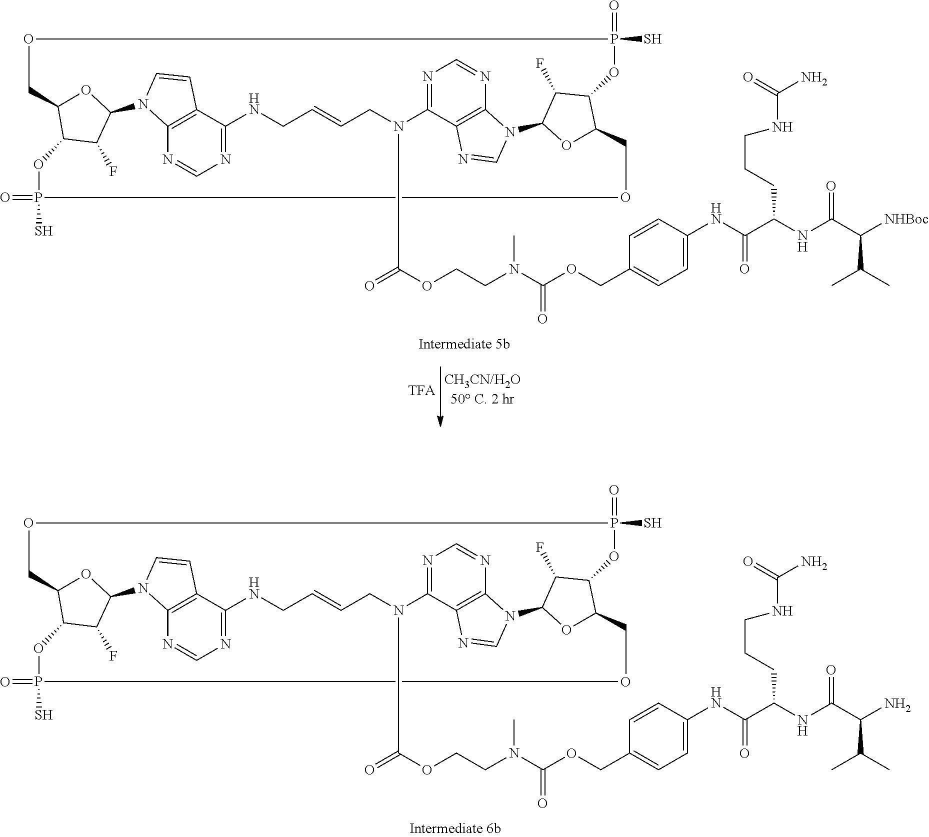

To a solution of Intermediate 5b (50 mg, 37.0 μmol, 1.0 eq) in acetonitrile (0.5 mL) and H₂O (0.5 mL) was added TFA (211 mg, 1.85 mmol, 137 μL, 50 eq). The mixture was stirred at 50° C. for 2 hrs. The mixture was concentrated in vacuo. The residue was purified by prep-HPLC (column: C₁₈₋₄ 150·30 mm·5 μm; mobile phase: [A: 0.1 M water (TFA) B: acetonitrile]; B %: 10%-40%, 20 min) to afford desired product (20 mg, 14.6 μmol, 39.59% yield, TFA) as white solid.

### Intermediate Example 22

### Preparation of Intermediate 8B

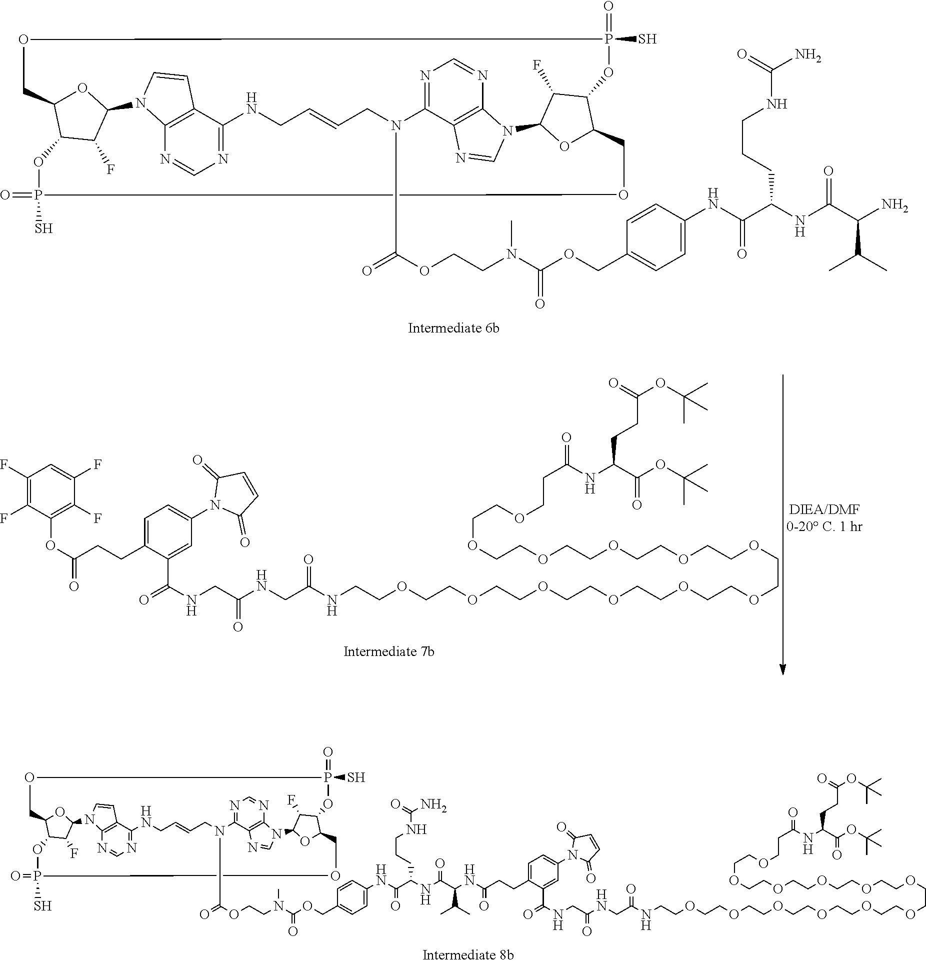

To a solution of Intermediate 6b (9.00 mg, 6.59 μmol, 1.0 eq, TFA) in DMF (0.1 mL) was added DIEA (2.55 mg, 19.8 μmol, 3.44 μL, 3.0 eq) and Intermediate 7b (9.18 mg, 6.59 μmol, 1 eq) at 0° C. The mixture was stirred at 20° C. for 1 hr. The pH of the mixture was adjusted to ˜6 with TFA at 0° C. The mixture was concentrated in vacuo to afford desired product (15 mg crude) as colorless oil.

### Intermediate Example 23

### Synthesis of 4-[[tert-butyl(dimethyl)silyl]oxymethyl]aniline

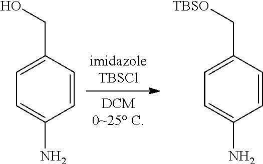 [SMILES: Nc1ccc(CO)cc1 ; CC(C)(C)[Si](C)(C)OCc1ccc(N)cc1]

To a solution of (4-aminophenyl)methanol (5.00 g, 40.6 mmol, 1.0 eq) in DCM (50 mL) was added imidazole (4.15 g, 60.9 mmol, 1.5 eq) and TBSCl (7.34 g, 48.7 mmol, 5.97 mL, 1.2 eq) at 0° C., and then stirred at 25° C. for 1 hr. The reaction mixture was quenched by addition H₂O 100 mL at 0° C., and then extracted with DCM (30 mL×3). The combined organic layers were washed with brine 50 mL, dried over Na₂SO₄, filtered and concentrated under reduced pressure to give a residue. The residue was purified by flash silica gel chromatography (Biotage®; 80 g SepaFlash® Silica Flash Column, Eluent of 50˜70% Ethyl acetate/petroleum ether gradient @ 80 mL/min). 4-[[tert-butyl(dimethyl)silyl]oxymethyl]aniline (9.6 g, 40.44 mmol, 99.60% yield) was obtained as colorless oil: ¹H NMR (400 MHz, CDCl₃) δ 7.13 (d, J=8.0 Hz, 2H), 6.67 (d, J=8.0 Hz, 2H), 4.64 (s, 2H), 0.94 (s, 9H), 0.10 (s, 6H); LC/MS [M+H] 238.2 (calculated); LC/MS [M+H] 238.1 (observed).

### Intermediate Example 24

### Synthesis of tert-butyl N-[(1S)-2-[4-[[tert-butyl(dimethyl)silyl]oxymethyl]anilino]-1-methyl-2-oxo-ethyl]carbamate

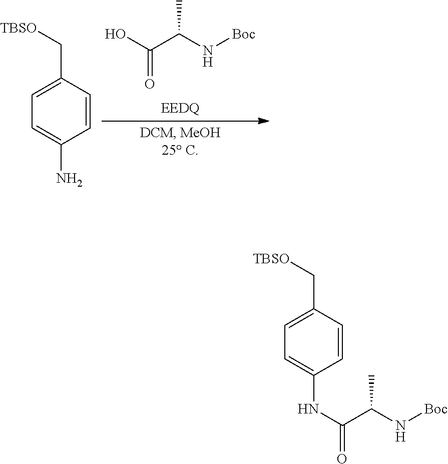 [SMILES: CC(C)(C)[Si](C)(C)OCc1ccc(N)cc1 ; C[C@H](NC(=O)OC(C)(C)C)C(=O)O ; C[C@H](NC(=O)OC(C)(C)C)C(=O)Nc1ccc(CO[Si](C)(C)C(C)(C)C)cc1]

To a solution of 4-[[tert-butyl(dimethyl)silyl]oxymethyl]aniline (9.34 g, 39.3 mmol, 1 eq) and (2S)-2-(tert-butoxycarbonylamino)propanoic acid (8.93 g, 47.2 mmol, 1.2 eq) in DCM (50 mL) and methanol (50 mL) was added EEDQ (29.2 g, 118 mmol, 3.0 eq), and then stirred at 25° C. for 1 hr. The reaction mixture was quenched by addition H₂O 100 mL at 0° C., then extracted with ethyl acetate (50 mL×3). The combined organic layers were washed with brine 50 mL, dried over Na₂SO₄, filtered and concentrated under reduced pressure to give a residue. The residue was purified by flash silica gel chromatography (Biotage®; 120 g SepaFlash® Silica Flash Column, Eluent of 15˜30% Ethyl acetate/petroleum ether gradient @ 80 mL/min). tert-butyl N-[(1S)-2-[4-[[tert-butyl(dimethyl)silyl]oxymethyl]anilino]-1-methyl-2-oxo-ethyl]carbamate (25 g, crude) was obtained as orange oil.

### Intermediate Example 25

### Synthesis of tert-butyl N-[(2S)-2-[bis(tert-butoxycarbonyl)amino]propanoyl]-N-[4-[[tert-butyl(dimethyl)silyl]oxymethyl]phenyl]carbamate

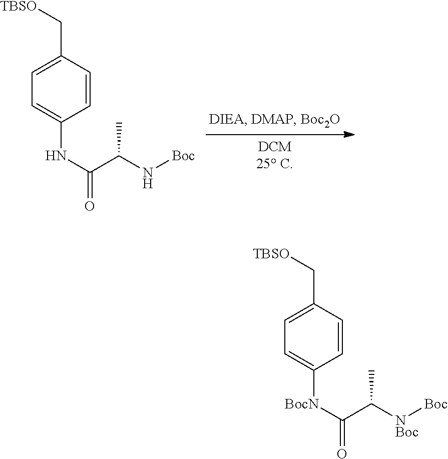 [SMILES: C[C@H](NC(=O)OC(C)(C)C)C(=O)Nc1ccc(CO[Si](C)(C)C(C)(C)C)cc1 ; C[C@@H](C(=O)N(C(=O)OC(C)(C)C)c1ccc(CO[Si](C)(C)C(C)(C)C)cc1)N(C(=O)OC(C)(C)C)C(=O)OC(C)(C)C]

To a solution of tert-butyl N-[(1S)-2-[4-[[tert-butyl(dimethyl)silyl]oxymethyl]anilino]-1-methyl-2-oxo-ethyl]carbamate (10.0 g, 24.5 mmol, 1.0 eq) in DCM (100 mL) was added DMAP (2.99 g, 24.5 mmol, 1.0 eq), DIEA (9.49 g, 73.4 mmol, 12.8 mL, 3.0 eq) and Boc₂O (16.0 g, 73.4 mmol, 16.9 mL, 3.0 eq). The mixture was stirred at 40° C. for 24 hrs. The reaction mixture was quenched by addition H₂O 100 mL at 0° C., and then extracted with DCM (50 mL×2). The combined organic layers were washed with brine 100 mL, dried over Na₂SO₄, filtered and concentrated under reduced pressure to give a residue. The residue was purified by flash silica gel chromatography (Biotage®; 120 g SepaFlash® Silica Flash Column, Eluent of 10˜20% Ethyl acetate/petroleum ether gradient @ 80 mL/min). Tert-butyl N-[(2S)-2-[bis(tert-butoxycarbonyl)amino]propanoyl]-N-[4-[[tert-butyl(dimethyl)silyl]oxymethyl]phenyl]carbamate (4.70 g, 7.72 mmol, 31.54% yield) was obtained as colorless oil: ¹H NMR (CDCl₃, 400 MHz) δ 7.31 (d, J=8.4 Hz, 2H), 7.11 (d, J=8.4 Hz, 2H), 5.48-5.43 (m, 1H), 4.75 (s, 2H), 1.55-1.50 (m, 27H), 1.02 (d, J=6.4 Hz, 3H), 0.94 (s, 9H), 0.08 (s, 6H); LC/MS [M+Na] 631.35 (calculated); LC/MS [M+Na] 631.3 (observed).

### Intermediate Example 26

### Synthesis of tert-butyl N-[(2S)-2-[bis(tert-butoxycarbonyl)amino]propanoyl]-N-[4-(hydroxy methyl)phenyl]carbamate

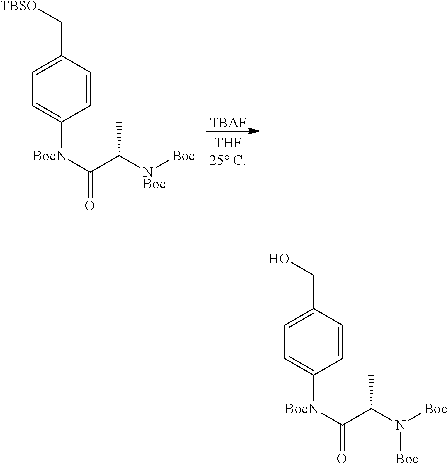 [SMILES: C[C@@H](C(=O)N(C(=O)OC(C)(C)C)c1ccc(CO[Si](C)(C)C(C)(C)C)cc1)N(C(=O)OC(C)(C)C)C(=O)OC(C)(C)C ; "TBAF" ; C[C@@H](C(=O)N(C(=O)OC(C)(C)C)c1ccc(CO)cc1)N(C(=O)OC(C)(C)C)C(=O)OC(C)(C)C]

To a solution of tert-butyl N-[(2S)-2-[bis(tert-butoxycarbonyl)amino]propanoyl]-N-[4-[[tert-butyl(dimethyl)silyl]oxymethyl]phenyl]carbamate (4.68 g, 7.69 mmol, 1.0 eq) in THF (50 mL) was added TBAF (1 M, 15.4 mL, 2.0 eq), and then stirred at 25° C. for 1 hr. The reaction mixture was quenched by addition H₂O (100 mL) at 0° C., then extracted with ethyl acetate (50 mL×3). The combined organic layers were washed with brine 50 mL, dried over Na₂SO₄, filtered and concentrated under reduced pressure to give a residue. The residue was purified by flash silica gel chromatography (Biotage®; 40 g SepaFlash® Silica Flash Column, Eluent of 50˜70% Ethyl acetate/petroleum ether gradient @ 60 mL/min). Tert-butyl N-[(2S)-2-[bis(tert-butoxycarbonyl)amino]propanoyl]-N-[4-(hydroxy methyl)phenyl]carbamate (2.10 g, 4.25 mmol, 55.24% yield) was obtained as colorless oil: ¹H NMR (400 MHz, CDCl₃) δ 7.36 (d, J=8.4 Hz, 2H), 7.15 (d, J=8.4 Hz, 2H), 5.49-5.44 (m, 1H), 4.70 (d, J=2.8 Hz, 2H), 1.53 (s, 21H), 1.35 (s, 9H); LC/MS [M+H] 517.2 (calculated); LC/MS [M+H] 517.2 (observed).

### Intermediate Example 27

### Synthesis of [4-[[(2S)-2-[bis(tert-butoxycarbonyl)amino]propanoyl]-tert-butoxycarbonyl-amino]phenyl]methyl (4-nitrophenyl) carbonate

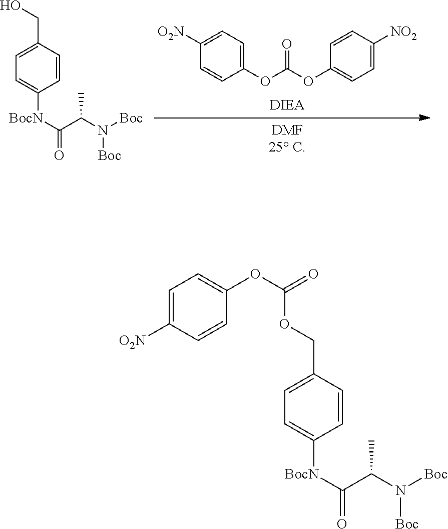 [SMILES: C[C@@H](C(=O)N(C(=O)OC(C)(C)C)c1ccc(CO)cc1)N(C(=O)OC(C)(C)C)C(=O)OC(C)(C)C ; O=C(Oc1ccc([N+](=O)[O-])cc1)Oc1ccc([N+](=O)[O-])cc1 ; C[C@@H](C(=O)N(C(=O)OC(C)(C)C)c1ccc(COC(=O)Oc2ccc([N+](=O)[O-])cc2)cc1)N(C(=O)OC(C)(C)C)C(=O)OC(C)(C)C]

To a solution of tert-butyl N-[(2S)-2-[bis(tert-butoxycarbonyl)amino]propanoyl]-N-[4-(hydroxymethyl)phenyl]carbamate (1.00 g, 2.02 mmol, 1.0 eq) in DCM (15 mL) was added DIEA (784 mg, 6.07 mmol, 1.06 mL, 3.0 eq) and bis(4-nitrophenyl) carbonate (1.23 g, 4.04 mmol, 2.0 eq), and then stirred at 25° C. for 1 hr. The reaction mixture was quenched by addition H₂O (30 mL) at 0° C., and then extracted with DCM (10 mL×3). The combined organic layers were washed with brine 20 mL, dried over Na₂SO₄, filtered and concentrated under reduced pressure to give a residue. The residue was purified by flash silica gel chromatography (Biotage®; 20 g SepaFlash® Silica Flash Column, Eluent of 10˜30% Ethyl acetate/petroleum ether gradient @ 40 mL/min). [4-[[(2S)-2-[bis(tert-butoxycarbonyl)amino]propanoyl]-tert-butoxycarbonyl-amino]phenyl]methyl (4-nitrophenyl) carbonate (1.71 g, crude) was obtained as colorless oil: LC/MS [M+Na] 682.3 (calculated); LC/MS [M+Na] 682.2 (observed).

### Intermediate Example 28

### Synthesis of tert-butyl N-[(2S)-2-[bis(tert-butoxycarbonyl)amino]propanoyl]-N-[4-(ethylcarbamoyloxymethyl)phenyl]carbamate

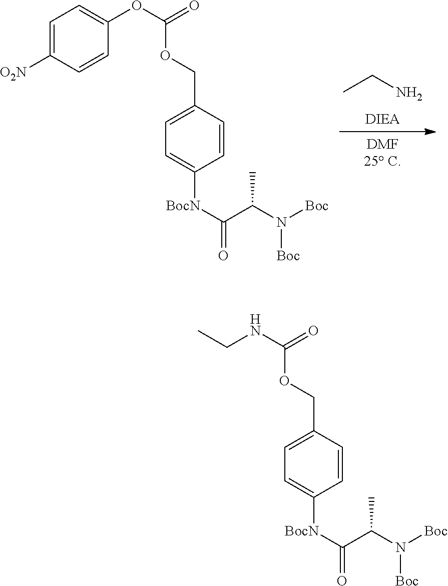

To a solution of ethanamine (315 mg, 3.87 mmol, 457 μL, 1.5 eq, HCl) in DMF (15 mL) was added DIEA (1.67 g, 12.9 mmol, 2.24 mL, 5.0 eq) and [4-[[(2S)-2-[bis(tert-butoxycarbonyl)amino]propanoyl]-tert-butoxycarbonyl-amino]phenyl]methyl (4-nitrophenyl) carbonate (1.70 g, 2.58 mmol, 1.0 eq) at 0° C. The mixture was stirred at 25° C. for 1 hr. The reaction mixture was quenched by addition H₂O (20 mL) at 0° C., then extracted with ethyl acetate (20 mL×3). The combined organic layers were washed with brine 20 mL, dried over Na₂SO₄, filtered and concentrated under reduced pressure to give a residue. The residue was purified by flash silica gel chromatography (Biotage®; 20 g SepaFlash® Silica Flash Column, Eluent of 30˜50% Ethyl acetate/petroleum ether gradient @ 40 mL/min). Tert-butyl N-[(2S)-2-[bis(tert-butoxycarbonyl)amino]propanoyl]-N-[4-(ethylcarbamoyloxymethyl)phenyl]carbamate (1.14 g, 2.02 mmol, 78.21% yield) was obtained as a white solid: LC/MS [M+Na] 588.3 (calculated); LC/MS [M+Na] 588.3 (observed).

### Intermediate Example 29

### Synthesis of tert-butyl N-[(2S)-2-[bis(tert-butoxycarbonyl)amino]propanoyl]-N-[4-[[chloromethyl(ethyl)carbamoyl]oxymethyl]phenyl]carbamate

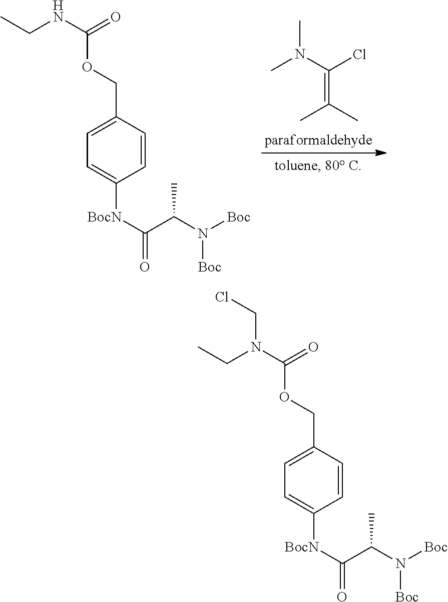

To a solution of tert-butyl N-[(2S)-2-[bis(tert-butoxycarbonyl)amino]propanoyl]-N-[4-(ethylcarbamoyloxymethyl)phenyl]carbamate (1.00 g, 1.77 mmol, 1.0 eq) in toluene (10 mL) was added paraformaldehyde (3.15 g, 2.65 mmol, 1.5 eq) and 1-chloro-N,N,2-trimethyl-prop-1-en-1-amine (1.18 g, 8.84 mmol, 1.17 mL, 5.0 eq). The mixture was stirred at 80° C. for 1 hr. The reaction mixture was filtered and concentrated under reduced pressure to give a residue. Compound tert-butyl N-[(2S)-2-[bis(tert-butoxycarbonyl)amino]propan oyl]-N-[4-[[chloromethyl(ethyl)carbamoyl]oxymethyl]phenyl]carbamate (2 g, crude) was obtained as colorless oil.

### Intermediate Example 30

### Synthesis of tert-butyl N-[(2S)-2-[bis(tert-butoxycarbonyl)amino]propanoyl]-N-[4-[[[2-[(1 S,2S,4R,6R,8S,9S,11S,12R,13S,19S)-12,19-difluoro-6-(3-fluorophenyl)-11-hydroxy-9,13-dimethyl-16-oxo-5,7-dioxapentacyclo[10.8.0.02,9.04,8.013,18]icosa-14,17-dien-8-yl]-2-oxo-ethoxy]methyl-ethyl-carbamoyl]oxymethyl]phenyl]carbamate

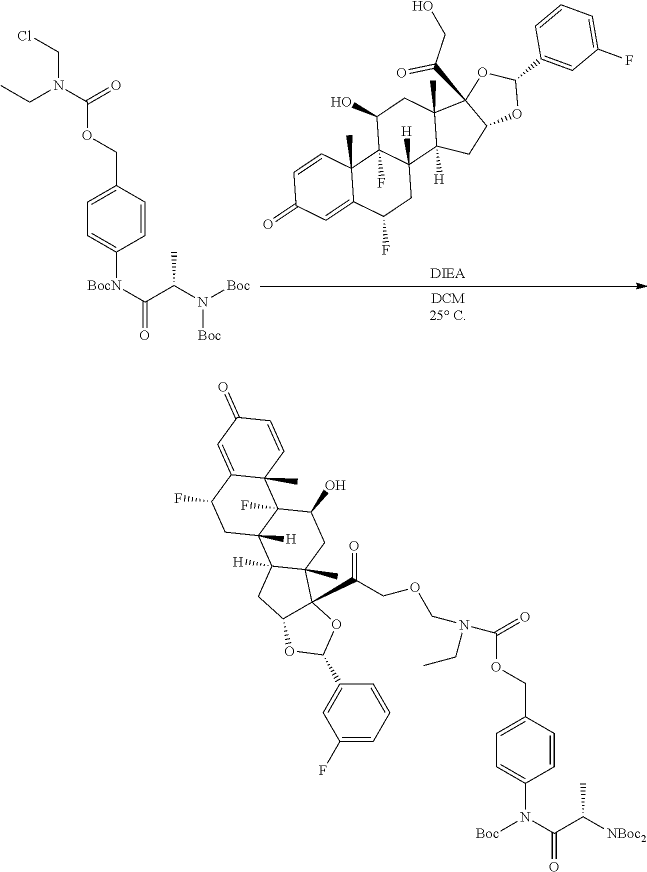

To a solution of tert-butyl N-[(2S)-2-[bis(tert-butoxycarbonyl)amino]propanoyl]-N-[4-[[chloromethyl(ethyl)carbamoyl]oxymethyl]phenyl]carbamate (1.18 g, 1.93 mmol, 2.0 eq) in DCM (15 mL) was added DIEA (371 mg, 2.87 mmol, 0.5 mL, 2.98 eq) and (1S,2S,4R,6R,8S,9S,11S,12R,13S,19S)-12,19-difluoro-6-(3-fluorophenyl)-11-hydroxy-8-(2-hydroxyacetyl)-9,13-dimethyl-5,7-dioxapentacyclo[10.8.0.02,9.04,8.013,18]icosa-14,17-dien-16-one (500 mg, 964.28 μmol, 1.0 eq), and then stirred at 25° C. for 12 hrs. The reaction mixture was quenched by addition H₂O (50 mL) at 0° C., and then extracted with DCM (20 mL×3). The combined organic layers were washed with brine 20 mL, dried over Na₂SO₄, filtered and concentrated under reduced pressure to give a residue. The residue was purified by flash silica gel chromatography (Biotage®; 20 g SepaFlash® Silica Flash Column, Eluent of 50-70% Ethyl acetate/petroleum ether gradient @ 45 mL/min). Tert-butyl N-[(2S)-2-[bis(tert-butoxycarbonyl)amino]propanoyl]-N-[4-[[[2-[(1 S,2S,4R,6R,8S,9S,11S,12R,13S,19S)-12,19-difluoro-6-(3-fluorophenyl)-11-hydroxy-9,13-dimethyl-16-oxo-5,7-dioxapentacyclo[10.8.0.02,9.04,8.013,18]icosa-14,17-dien-8-yl]-2-oxo-ethoxy]methyl-ethyl-carbamoyl]oxymethyl]phenyl]carbamate (658 mg, 600.26 μmol, 62.25% yield) was obtained as colorless oil: LC/MS [M+Na] 1118.5 (calculated); LC/MS [M+Na] 1118.3 (observed).

### Intermediate Example 31

### Synthesis of [4-[[(2S)-2-aminopropanoyl]amino] phenyl]methylN-[[2-[(1S,2S,4R,6R,8S,9S,11S,12R,13S,19S)-12,19-difluoro-6-(3-fluorophenyl)-11-hydroxy-9,13-dimethyl-16-oxo5,7dioxapentacyclo[10.8.0.02,9.04,8.013,18]icosa-14,17-dien-8-yl]-2-oxo-ethoxy]methyl]-N-ethyl-carbamate

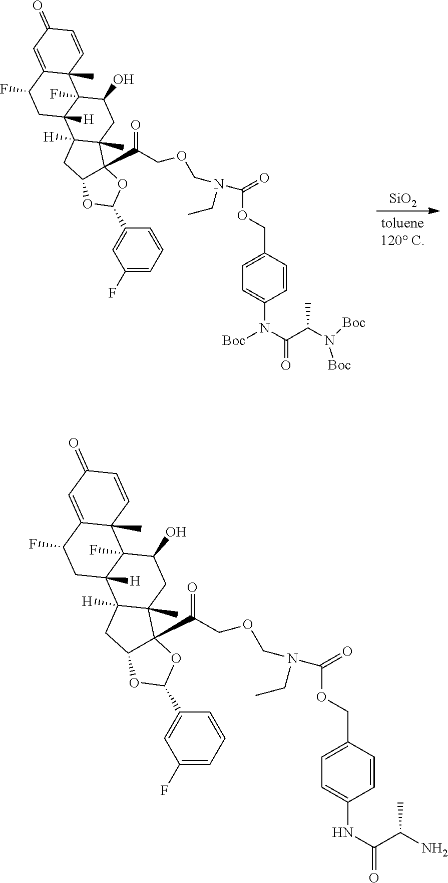

To a solution of tert-butyl N-[(2S)-2-[bis(tert-butoxycarbonyl)amino]propanoyl]-N-[4-[[[2-[(1S,2S,4R,6R,8S,9S,11S,12R,13S,19S)-12,19-difluoro-6-(3-fluorophenyl)-11-hydroxy-9,13-dimethyl-16-oxo-5,7-dioxapentacyclo[10.8.0.02,9.04,8.013,18]icosa-14,17-dien-8-yl]-2-oxo-ethoxy]methyl-ethyl-carbamoyl]oxymethyl]phenyl]carbamate (0.96 g, 875 μmol, 1.0 eq) in toluene (15 mL) was added SiO₂ (10.0 g, 166 mmol, 190 eq). The mixture was stirred at 120° C. for 1 hr. The reaction mixture was filtered, SiO₂ was washed with methanol (20 mL×2), and the filtrate was concentrated under reduced pressure to give a residue. [4-[[(2S)-2-aminopropanoyl]amino] phenyl]methylN-[[2-[(1S,2S,4R,6R,8S,9S,11S,12R,13S,19S)-12,19-difluoro-6-(3-fluorophenyl)-11-hydroxy-9,13-dimethyl-16-oxo5,7dioxapentacyclo[10.8.0.02,9.04,8.013,18]icosa-14,17-dien-8-yl]-2-oxo-ethoxy]methyl]-N-ethyl-carbamate (500 mg, 628.27 μmol, 71.74% yield) was obtained as colorless oil: LC/MS [M+H] 796.3 (calculated); LC/MS [M+H] 796.4 (observed).

### Intermediate Example 32

### Synthesis of [4-[[(2S)-2-[[(2S)-2-amino-3-methyl-butanoyl]amino]propanoyl]amino]phenyl]methyl N-[[2-[(1S,2S,4R,6R,8S,9S,11S,12R,13S,19S)-12,19-difluoro-6-(3-fluorophenyl)-11-hydroxy-9,13-dimethyl-16-oxo-5,7-dioxapentacyclo[10.8.0.02,9.04,8.013,18]icosa-14,17-dien-8-yl]-2-oxo-ethoxy]methyl]-N-ethyl-carbamate

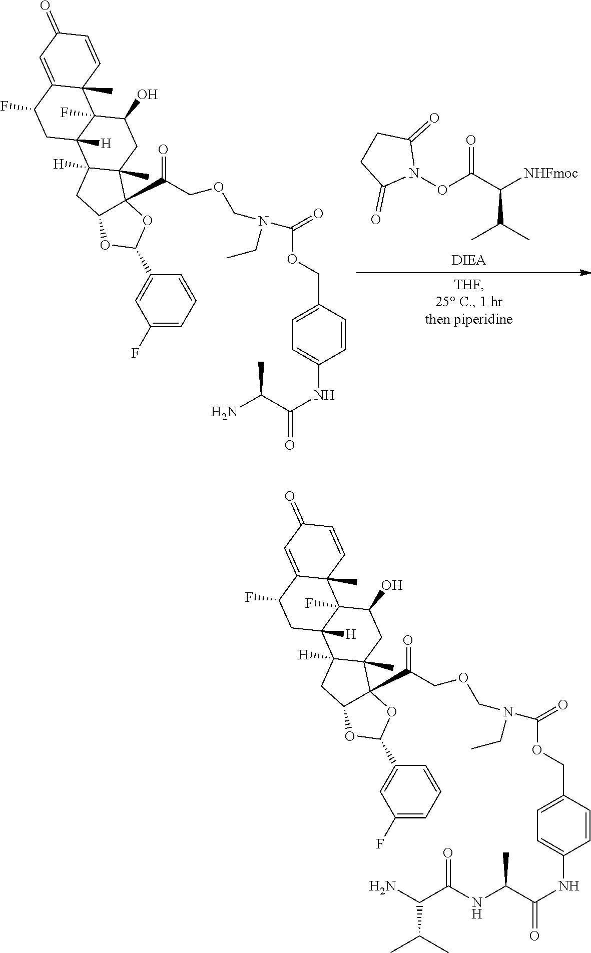

To a solution of [4-[[(2S)-2-aminopropanoyl]amino]phenyl]methyl N-[[2-[(1S,2S,4R,6R,8S,9S,11S,12R,13S,19S)-12,19-difluoro-6-(3-fluorophenyl)-11-hydroxy-9,13-dimeth yl-16-oxo-5,7-dioxapentacyclo[10.8.0.02,9.04,8.013,18]icosa-14,17-dien-8-yl]-2-oxo-ethoxy]methyl]-N-ethyl-carbamate (200 mg, 251 μmol, 1.0 eq) in THF (2 mL) was added DIEA (97.4 mg, 754 μmol, 131 μL, 3.0 eq) and (2,5-dioxopyrrolidin-1-yl) (2S)-2-(9H-fluoren-9-ylmethoxycarbonylamino)-3-methyl-butanoate (164 mg, 377 μmol, 1.5 eq), and then stirred at 25° C. for 1 hr. piperidine (86.2 mg, 1.01 mmol, 0.10 mL, 5.66 eq) was added. The mixture was stirred at 25° C. for another 1 hr. The mixture was purified by prep-HPLC (column: Phenomenex Luna 80×30 mm×3 μm; mobile phase: [water (TFA)-ACN]; B %: 30%-65%, 8 min). [4-[[(2S)-2-[[(2S)-2-amino-3-methyl-butanoyl]amino]propanoyl]amino]phenyl]methyl N-[[2-[(1S,2S,4R,6R,8S,9S,11S,12R,13S,19S)-12,19-difluoro-6-(3-fluorophenyl)-11-hydroxy-9,13-dimethyl-16-oxo-5,7-dioxapentacyclo[10.8.0.02,9.04,8.013,18]icosa-14,17-dien-8-yl]-2-oxo-ethoxy]methyl]-N-ethyl-carbamate (100 mg, 44.69 μmol, 24.97% yield, 40% purity) was obtained as a light yellow solid: LC/MS [M+H] 895.4 (calculated); LC/MS [M+H] 895.5 (observed).

### Intermediate Example 33

### Synthesis of tert-butyl (((9H-fluoren-9-yl)methoxy)carbonyl)-L-alanyl-L-alanylglycinate

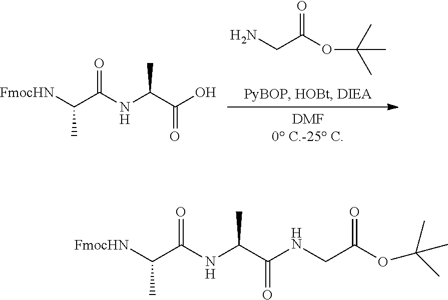

To a stirred solution of (2S)-2-[(2S)-2-{[(9H-fluoren-9-ylmethoxy) carbonyl]amino} propanamido] propanoic acid (1.0 g, 2.6 mmol, 1.0 eq.) in N,N-dimethylformamide (5.0 mL) with an inert atmosphere of nitrogen, was added tert-butyl 2-aminoacetate (343.0 mg, 2.6 mmol, 1.0 eq.), N,N-diisopropylethylamine (675.9 mg, 5.2 mmol, 2.0 eq.), benzotriazol-1-yl-oxytripyrrolidinophosphonium hexafluorophosphate (530.0 mg, 3.9 mmol, 1.5 eq.) at 0° C. The resulting solution was stirred for 4 h at 25° C., diluted with water (100 mL). The resulting mixture was extracted with dichloromethane (3×100 mL) and organic layers were washed with saturated sodium chloride solution (3×100 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 1.0 g (77%) of tert-butyl (((9H-fluoren-9-yl)methoxy)carbonyl)-L-alanyl-L-alanylglycinate was obtained as a white solid. MS m/z [M+H]⁺ (ESI): 496.23.

### Intermediate Example 34

### Synthesis of (((9H-fluoren-9-yl)methoxy)carbonyl)-L-alanyl-L-alanylglycine

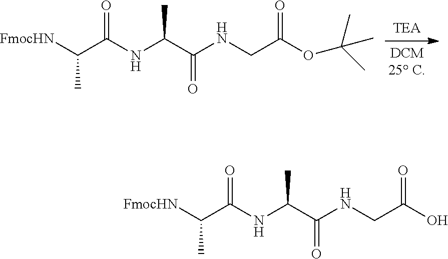

To a solution of tert-butyl (((9H-fluoren-9-yl)methoxy)carbonyl)-L-alanyl-L-alanylglycinate (1.0 g, 2.0 mmol, 1.0 eq.) in dichloromethane (20.0 mL) with an inert atmosphere of nitrogen, was added trifluoroacetic acid (10.0 mL) at 25° C. The resulting solution was stirred for 5 h at 25° C. and concentrated under vacuum. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 800 mg (90%) of (((9H-fluoren-9-yl)methoxy)carbonyl)-L-alanyl-L-alanylglycine was obtained as a yellow oil. MS m/z [M−H]⁻ (ESI): 438.17.

### Intermediate Example 35

### Synthesis of (5S,8S)-1-(9H-fluoren-9-yl)-5,8-dimethyl-3,6,9-trioxo-2-oxa-4,7,10-triazaundecan-11-yl acetate

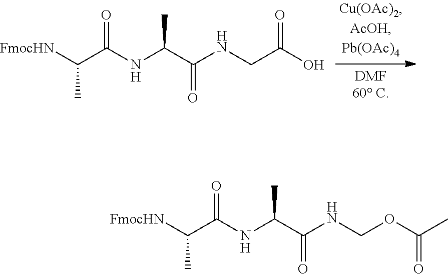

To a stirred solution of (((9H-fluoren-9-yl)methoxy)carbonyl)-L-alanyl-L-alanylglycine (800.0 mg, 1.8 mmol, 1.0 eq.) and cupric acetate monohydrate (33.0 mg, 0.2 mmol, 0.1 eq.) in N,N-Dimethylformamide (10.0 mL) were added acetic acid (164.0 mg, 2.7 mmol, 1.5 eq.) and lead tetraacetate (4.0 g, 9.0 mmol, 5.0 eq.) at room temperature under air atmosphere. The resulting mixture was stirred for additional 5 h at 60° C. The resulting solution was cooled to 25° C., diluted with water (100 mL). The resulting mixture was extracted with dichloromethane (3×100 mL) and organic layers were washed with saturated sodium chloride solution (3×100 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 425 mg (51%) of (5S,8S)-1-(9H-fluoren-9-yl)-5,8-dimethyl-3,6,9-trioxo-2-oxa-4,7,10-triazaundecan-11-yl acetate was obtained as a white solid. MS m/z [M+Na]⁺ (ESI): 476.17.

### Intermediate Example 36

### Synthesis of (5S,8S)-1-(9H-fluoren-9-yl)-5,8-dimethyl-3,6,9-trioxo-2-oxa-4,7,10-triazaundecan-11-yl acetate

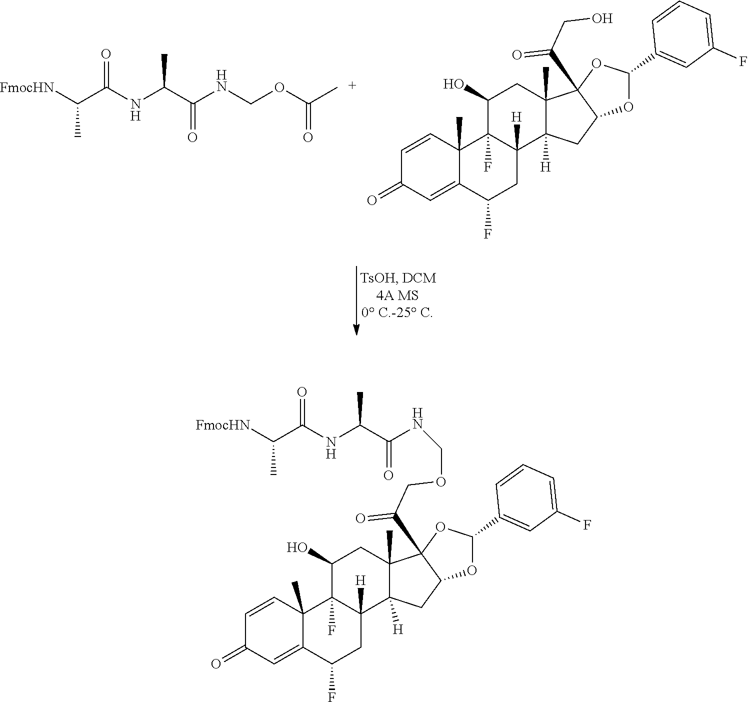

To a stirred solution of (5S,8S)-1-(9H-fluoren-9-yl)-5,8-dimethyl-3,6,9-trioxo-2-oxa-4,7,10-triazaundecan-11-yl acetate (425.0 mg, 0.94 mmol, 1.0 eq.) and (2S,6aS,6bR,7S,8aS,8bS,10R,11aR,12aS,12bS)-2,6b-difluoro-10-(3-fluorophenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (243.0 mg, 0.5 mmol, 0.5 eq.) in dichloromethane (5.0 mL) were added p-toluenesulfonic acid (32.2 mg, 0.2 mmol, 0.20 eq.) in portions at 0° C. under air atmosphere. The resulting mixture was stirred for additional 12 h at 25° C. The crude product was purified by reverse phase flash with the following conditions (column, C₁₈ silica gel; mobile phase, acetonitrile in water (10 mmol/L ammonium bicarbonate), 10% to 100% gradient in 20 min; detector, UV 254 nm.). 200 mg (47%) of (9H-fluoren-9-yl)methyl ((S)-1-(((S)-1-(((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)amino)-1-oxopropan-2-yl)amino)-1-oxopropan-2-yl)carbamate was obtained as a white solid. MS m/z [M+H]⁺ (ESI): 912.36.

### Intermediate Example 37

### Synthesis of (5S,8S)-1-(9H-fluoren-9-yl)-5,8-dimethyl-3,6,9-trioxo-2-oxa-4,7,10-triazaundecan-11-yl acetate

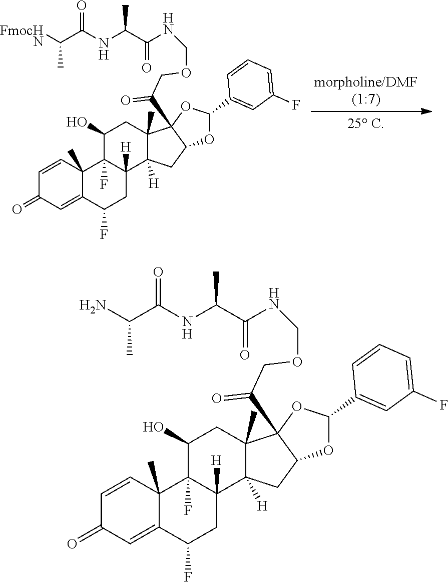

To a stirred solution of (9H-fluoren-9-yl)methyl ((S)-1-(((S)-1-(((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)amino)-1-oxopropan-2-yl)amino)-1-oxopropan-2-yl)carbamate (200 mg, 0.2 mmol, 1.0 eq.) in N, N-dimethylformamide (2.0 mL) were added morpholine (0.3 mL) at 25° C. under air atmosphere. The resulting mixture was stirred for additional 1 h at 25° C. The crude product was purified by reverse phase flash with the following conditions (column, C₁₈ silica gel; mobile phase, acetonitrile in water (0.1% trifluoroacetic acid), 10% to 100% gradient in 20 min; detector, UV 254 nm). 100 mg (66%) of (S)-2-amino-N—((S)-1-(((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)amino)-1-oxopropan-2-yl)propanamide was obtained as a white solid. MS m/z [M+H]⁺ (ESI): 690.29.

### Intermediate Example 38

### Synthesis of tert-butyl (((9H-fluoren-9-yl)methoxy)carbonyl)-L-phenylalanylglycinate

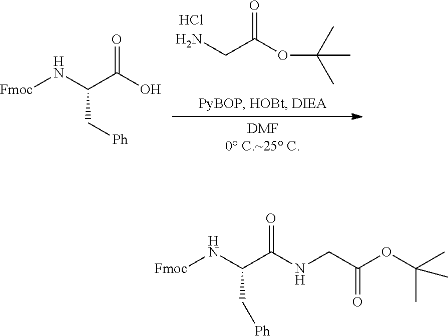

To a solution of (2S)-2-{[(9H-fluoren-9-ylmethoxy)carbonyl]amino}-3-phenylpropanoic acid (5.0 g, 12.9 mmol, 1.0 eq.) in N,N-dimethylformamide (50.0 mL) with an inert atmosphere of nitrogen, was added tert-butyl 2-aminoacetate hydrochloride (2.6 g, 15.5 mmol, 1.2 eq.), benzotriazol-1-yl-oxytripyrrolidinophosphonium hexafluorophosphate (10.1 g, 19.4 mmol, 1.5 eq.), 1-hydroxybenzotriazole (2.6 g, 19.4 mmol, 1.5 eq.), N,N-diisopropylethylamine (4.9 g, 38.7 mmol, 3.0 eq.) at 0° C. The resulting solution was stirred for 4 h at 25° C., diluted with water (1000 mL). The resulting mixture was extracted with dichloromethane (3×1000 mL) and organic layers were washed with saturated sodium chloride solution (3×1000 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 5.2 g (80%) of tert-butyl (((9H-fluoren-9-yl)methoxy)carbonyl)-L-phenylalanylglycinate was obtained as white solid. MS m/z [M+H]⁺ (ESI): 501.23.

### Intermediate Example 39

### Synthesis of tert-butyl L-phenylalanylglycinate

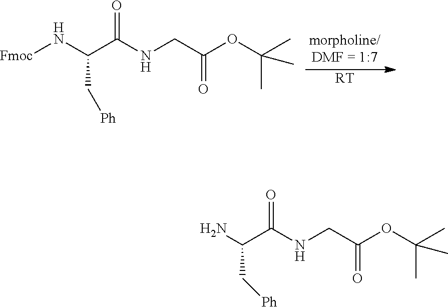

To a solution of tert-butyl (((9H-fluoren-9-yl)methoxy)carbonyl)-L-phenylalanylglycinate (5.2 g, 10.4 mmol, 1.0 eq.) in morpholine/N,N-dimethylformamide (7.0 mL/49.0 mL) with an inert atmosphere of nitrogen, the resulting solution was stirred for 1 h at 25° C., diluted with water (1000 mL). The resulting mixture was extracted with dichloromethane (3×1000 mL) and organic layers were washed with saturated sodium chloride solution (3×1000 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 2.7 g (93%) of tert-butyl L-phenylalanylglycinate was obtained as white solid. MS m/z [M+H]⁺ (ESI): 279.16.

### Intermediate Example 40

### Synthesis of tert-butyl (((9H-fluoren-9-yl)methoxy)carbonyl)glycylglycyl-L-phenylalanylglycinate

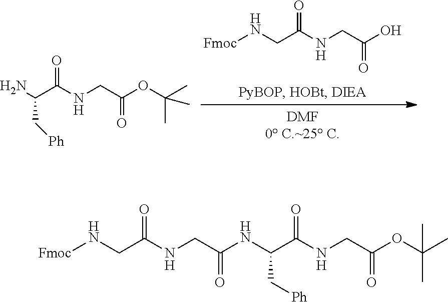

To a solution of tert-butyl L-phenylalanylglycinate (2.7 g, 9.7 mmol, 1.0 eq.) in N,N-dimethylformamide (30.0 mL) with an inert atmosphere of nitrogen, was added (2-{[(9H-fluoren-9-ylmethoxy)carbonyl]amino}acetamido)acetic acid (3.4 g, 9.7 mmol, 1.0 eq.), benzotriazol-1-yl-oxytripyrrolidinophosphonium hexafluorophosphate (7.6 g, 14.6 mmol, 1.5 eq.), 1-hydroxybenzotriazole (1.9 g, 14.6 mmol, 1.5 eq.), N,N-diisopropylethylamine (3.8 g, 29.1 mmol, 3.0 eq.) at 0° C. The resulting solution was stirred for 4 h at 25° C., diluted with water (500 mL). The resulting mixture was extracted with dichloromethane (3×500 mL) and organic layers were washed with saturated sodium chloride solution (3×500 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 3.8 g (64%) of tert-butyl (((9H-fluoren-9-yl)methoxy)carbonyl)glycylglycyl-L-phenylalanylglycinate was obtained as white solid. MS m/z [M+H]⁺ (ESI): 615.27.

### Intermediate Example 40

### Synthesis of tert-butyl (((9H-fluoren-9-yl)methoxy)carbonyl)glycylglycyl-L-phenylalanylglycylglycinate

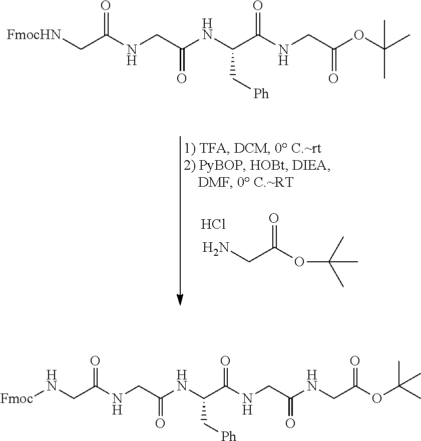

To a solution of tert-butyl (((9H-fluoren-9-yl)methoxy)carbonyl)glycylglycyl-L-phenylalanylglycinate (3.8 g, 6.2 mmol, 1.0 eq.) in dichloromethane (40.0 mL) with an inert atmosphere of nitrogen, was added trifluoroacetic acid (10.0 mL) at 0° C. The resulting solution was stirred for 5 h at 25° C. and concentrated under vacuum. To this solution in N,N-dimethylformamide (30.0 mL) was added tert-butyl 2-aminoacetate hydrochloride (1.0 g, 6.2 mmol, 1.0 eq.), benzotriazol-1-yl-oxytripyrrolidinophosphonium hexafluorophosphate (4.8 g, 9.3 mmol, 1.5 eq.), 1-hydroxybenzotriazole (1.3 g, 9.3 mmol, 1.5 eq.), N,N-diisopropylethylamine (2.4 g, 18.6 mmol, 3.0 eq.) at 0° C. The resulting solution was stirred for 4 h at 25° C., diluted with water (300 mL). The resulting mixture was extracted with dichloromethane (3×500 mL) and organic layers were washed with saturated sodium chloride solution (3×500 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 2.6 g (64%) of tert-butyl (((9H-fluoren-9-yl)methoxy)carbonyl)glycylglycyl-L-phenylalanylglycylglycinate was obtained as white solid. MS m/z [M+H]⁺ (ESI): 672.30; ¹H NMR (300 MHz, DMSO-d₆) δ: 1.39 (s, 9H), 2.75-2.82 (m, 1H), 3.03-3.09 (m, 1H), 3.56-3.80 (m, 8H), 4.19-4.31 (m, 3H), 4.49-4.57 (m, 1H), 7.15-7.24 (m, 7H), 7.25-7.44 (m, 2H), 7.57-7.61 (m, 1H), 7.69-7.72 (m, 2H), 7.88-7.99 (m, 2H), 8.01-8.15 (m, 3H), 8.31-8.35 (m, 1H).

### Intermediate Example 40

### Synthesis of (((9H-fluoren-9-yl)methoxy)carbonyl)glycylglycyl-L-phenylalanylglycylglycine

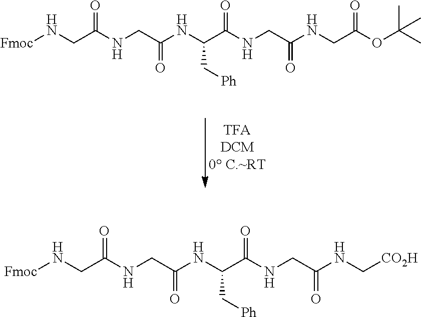

To a solution of tert-butyl (((9H-fluoren-9-yl)methoxy)carbonyl)glycylglycyl-L-phenylalanylglycylglycinate (2.6 g, 3.9 mmol, 1.0 eq.) in dichloromethane (30.0 mL) with an inert atmosphere of nitrogen, was added trifluoroacetic acid (15.0 mL) at 0° C. The resulting solution was stirred for 5 h at 25° C. and concentrated under vacuum. The crude product was purified by Flash-Prep-HPLC with the following conditions (IntelFlash-1): Column, C₁₈ silica gel; mobile phase, water (with 0.1% trifluoroacetic acid) and acetonitrile (10.0% acetonitrile up to 100.0% in 20 min); Detector, UV 254 nm. 2.1 g (88%) of (((9H-fluoren-9-yl)methoxy)carbonyl)glycylglycyl-L-phenylalanylglycylglycine was obtained as white solid. MS m/z [M+H]⁺ (ESI): 616.20.

### Intermediate Example 41

### Synthesis of (S)-11-benzyl-1-(9H-fluoren-9-yl)-3,6,9,12,15-pentaoxo-2-oxa-4,7,10,13,16-pentaazaheptadecan-17-yl acetate

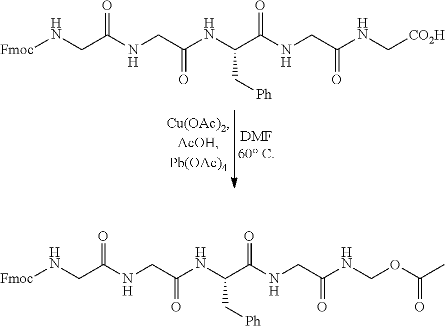

To a solution of (((9H-fluoren-9-yl)methoxy)carbonyl)glycylglycyl-L-phenylalanylglycylglycine (2.1 g, 3.4 mmol, 1.0 eq.) in N,N-dimethylformamide (30.0 mL) with an inert atmosphere of nitrogen, was added cupric acetate anhydrous (254 mg, 1.4 mmol, 0.4 eq.), acetic acid (468 mg, 7.8 mmol, 2.3 eq.), lead tetraacetate (1.8 g, 4.1 mmol, 1.2 eq.) at 25° C. The resulting mixture was stirred for 5 h at 60° C. The mixture was cooled down to 25° C., diluted with water (300 mL), extracted dichloromethane (3×500 mL) and organic layers were washed with saturated sodium chloride solution (3×500 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 870 mg (41%) of (S)-11-benzyl-1-(9H-fluoren-9-yl)-3,6,9,12,15-pentaoxo-2-oxa-4,7,10,13,16-pentaazaheptadecan-17-yl acetate was obtained as white solid. MS m/z [M+H]⁺ (ESI): 630.20.

### Intermediate Example 42

### Synthesis of (9H-fluoren-9-yl)methyl ((S)-10-benzyl-1-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-1,6,9,12,15-pentaoxo-3-oxa-5,8,11,14-tetraazahexadecan-16-yl)carbamate

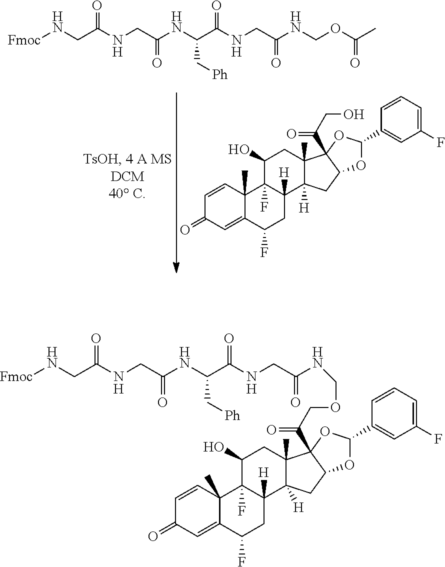

To a solution of (S)-11-benzyl-1-(9H-fluoren-9-yl)-3,6,9,12,15-pentaoxo-2-oxa-4,7,10,13,16-pentaazaheptadecan-17-yl (870.0 mg, 1.38 mmol, 3.0 eq.) in dichloromethane (8.0 mL) was added (2S,6aS,6bR,7S,8aS,8bS,10R,11aR,12aS,12bS)-2,6b-difluoro-10-(3-fluorophenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (238.8 mg, 0.46 mmol, 1.0 eq.) at 25° C. under nitrogen atmosphere followed by the addition of p-toluenesulfonic acid (31.7 mg, 0.18 mmol, 0.4 eq.) at 25° C. The resulting mixture was stirred for 12 h at 40° C. The mixture was cooled down to 25° C., diluted with water (300 mL), extracted dichloromethane (3×500 mL) and organic layers were washed with saturated sodium chloride solution (3×500 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 150.0 mg (30%) of (9H-fluoren-9-yl)methyl ((S)-10-benzyl-1-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-1,6,9,12,15-pentaoxo-3-oxa-5,8,11,14-tetraazahexadecan-16-yl)carbamate was obtained as white solid. MS m/z [M+H]⁺ (ESI): 1088.42.

### Intermediate Example 43

### Synthesis of (S)-2-(2-(2-aminoacetamido)acetamido)-N-(2-(((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)amino)-2-oxoethyl)-3-phenylpropanamide

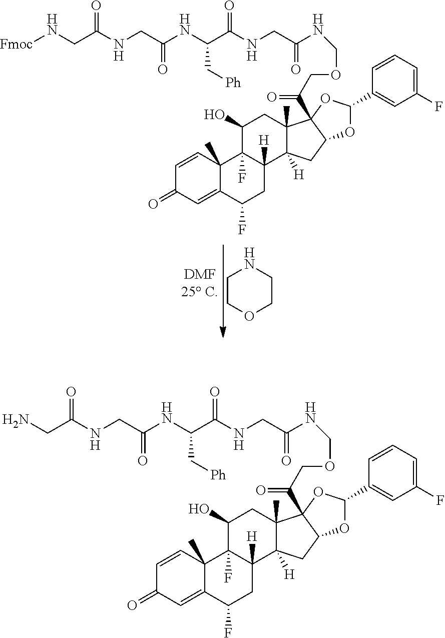

A solution of (9H-fluoren-9-yl)methyl ((S)-10-benzyl-1-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-1,6,9,12,15-pentaoxo-3-oxa-5,8,11,14-tetraazahexadecan-16-yl)carbamate (150.0 mg, 0.14 mmol, 1.0 eq.) in morpholine/N,N-dimethylformamide (0.1 mL/0.7 mL) with an inert atmosphere of nitrogen, the resulting solution was stirred for 1 h at 25° C., diluted with water (10 mL). The resulting mixture was extracted with dichloromethane (3×30 mL) and organic layers were washed with saturated sodium chloride solution (3×30 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 100.6 mg (84%) of (S)-2-(2-(2-aminoacetamido)acetamido)-N-(2-(((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)amino)-2-oxoethyl)-3-phenylpropanamide was obtained as white solid. MS m/z [M+H]⁺ (ESI): 866.35.

### Intermediate Example 44

### Synthesis of (2S,6aS,6bR,7S,8aS,8bS,10R,11aR,12aS,12bS)-2,6b-difluoro-10-(3-fluorophenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

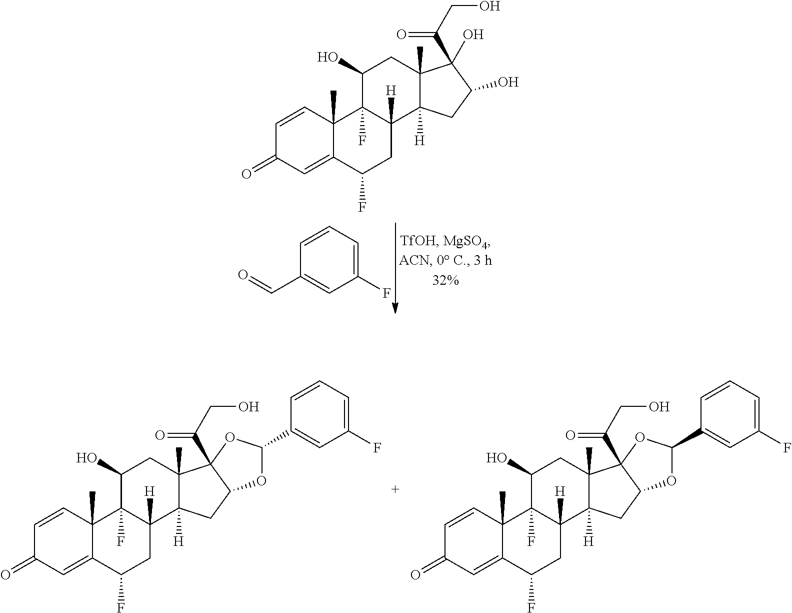

To a solution of fluocinolone (2.0 g, 4.85 mmol, 1.0 eq.) in acetonitrile (20.0 mL) with an inert atmosphere of nitrogen, was added magnesium sulfate (2.0 g, 16.9 mmol, 3.5 eq.) and 3-fluorobenzaldehyde (903.0 mg, 7.28 mmol, 1.5 eq.), followed by the addition of trifluoromethanesulfonic acid (2.2 g, 14.6 mmol, 3.0 eq.) at 0° C. The resulting solution was stirred for 3 h at 0° C., adjusted pH value to 7 with saturated sodium bicarbonate solution (1.0 mL). The mixture was diluted with 300 mL of water and extracted with 3×500 mL of dichloromethane. The organic layers were combined, washed with 3×500 mL of saturated sodium chloride solution, dried over anhydrous sodium sulfate, filtered and concentrated under reduced pressure. The crude product (>1 g) was purified by achiral-SFC with the following conditions: Column, Torus 2-PIC Column 4.6×100 mm, 5 μm; mobile phase: isopropyl alcohol (1% 2 mol/L NH₃-methanol) and CO₂; Detector, UV 254 nm. 800 mg (32%) of (2S,6aS,6bR,7S,8aS,8bS,10R,11aR,12aS,12bS)-2,6b-difluoro-10-(3-fluorophenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one was obtained as a white solid: MS m/z [M+H]⁺ (ESI): 519.20; ¹H NMR (400 MHz, Methanol-d₄) δ: 0.99 (s, 3H), 1.28-1.65 (m, 4H), 1.71-1.83 (m, 3H), 2.24-2.39 (m, 3H), 2.66-2.74 (m, 1H), 4.31-4.36 (m, 2H), 4.65 (d, J=19.6 Hz, 1H), 5.08-5.09 (m, 1H), 5.49-5.62 (m, 2H), 6.32-6.35 (m, 2H), 7.09-7.18 (m, 2H), 7.27-7.33 (m, 2H), 7.38-7.41 (m, 1H).

### Intermediate Example 45

### Synthesis of (9H-fluoren-9-yl)methyl (2-(((((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)(hydroxy)phosphoryl)oxy)(hydroxy)phosphoryl)oxy)ethyl)carbamate

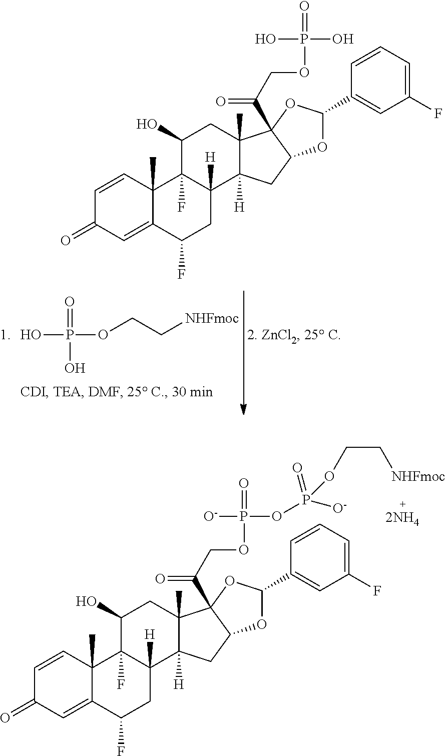

To a solution of (9H-fluoren-9-yl)methyl (2-(phosphonooxy)ethyl)carbamate (327.0 mg, 0.90 mmol, 1.5 eq.) in N,N-dimethylformamide (5.0 mL) with an inert atmosphere of nitrogen, was added trimethylamine (91.0 mg, 0.90 mmol, 1.2 eq.), 1,1′-carbonyldiimidazole (183.0 mg, 1.13 mmol, 1.5 eq.). The resulting mixture was stirred for 30 min at 25° C. To this mixture was added 2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl dihydrogen phosphate (450.0 mg, 0.75 mmol, 1.0 eq.) and zinc chloride (816.0 mg, 6.0 mmol, 8.0 eq.). The resulting mixture was stirred for overnight at 25° C., diluted with methanol (10.0 mL) and filtered. The filtrate was concentrated under reduced pressure. The crude product was purified by Flash with the following conditions: Column, C₁₈ silica gel; mobile phase, water (with 5 mmol/L ammonium hydroxide) and acetonitrile (10.0% acetonitrile up to 100.0% in 20 min); Detector, UV 254 nm. 310 mg of (9H-fluoren-9-yl)methyl (2-(((((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)(hydroxy)phosphoryl)oxy)(hydroxy)phosphoryl)oxy)ethyl)carbamate was obtained as white solid. MS m/z [M−H]⁻ (ESI): 942.20.

### Intermediate Example 46

### Synthesis of (2-(((((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)(hydroxy)phosphoryl)oxy)(hydroxy)phosphoryl)oxy)ethyl)carbamate

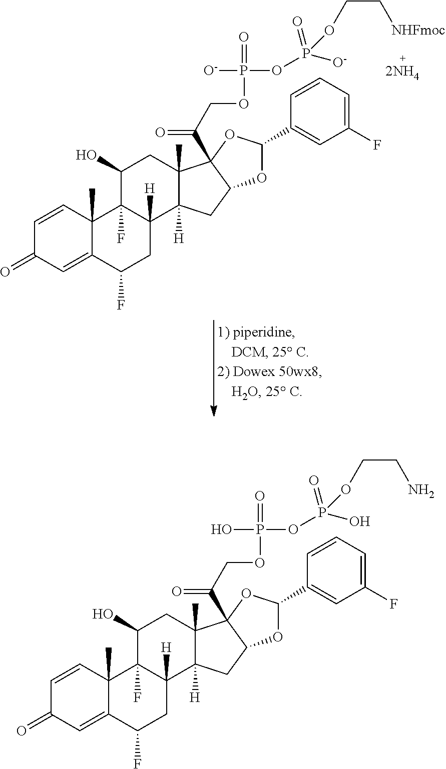

To a solution of (9H-fluoren-9-yl)methyl (2-(((((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)(hydroxy)phosphoryl)oxy)(hydroxy)phosphoryl)oxy)ethyl)carbamate (310.0 mg, 0.33 mmol, 1.0 eq.) in dichloromethane (5.0 mL) with an inert atmosphere of nitrogen, was added piperidine (196.0 mg, 2.31 mmol, 7.0 eq.). The resulting solution was stirred for 3 h at 25° C., concentrated under reduced pressure. The crude product was purified by Flash with the following conditions: Column, C₁₈ silica gel; mobile phase, water (with 5 mmol/L ammonium hydroxide) and acetonitrile (5.0% acetonitrile up to 100.0% in 20 min); Detector, UV 254 nm. The eluent was concentrated under reduced pressure. To the solution of desired product (ammonium salt) in water (5.0 mL) with an inert atmosphere of nitrogen, was added Dowex 50 w×8 (200.0 mg). The resulting solution was stirred for additional 1 h at 25° C., filtered. The filtrate was concentrated under reduced pressure. 115 mg (21% over two steps) of the desired product was obtained as white solid. MS m/z [M−H]⁻ (ESI): 720.20.

### Intermediate Example 47

### Synthesis of benzyl (E)-3-(4,4,5,5-tetramethyl-1,3,2-dioxaborolan-2-yl)acrylate

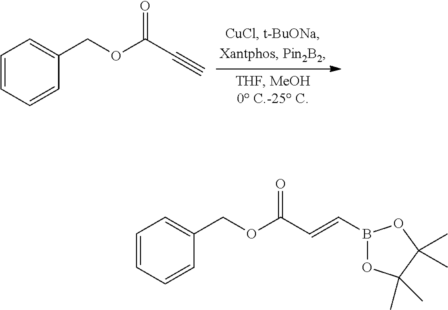 [SMILES: C#CC(=O)OCc1ccccc1 ; CC1(C)OB(/C=C/C(=O)OCc2ccccc2)OC1(C)C]

To a stirred solution of copper(I) chloride (0.09 g, 0.93 mmol, 0.03 eq.) and 4,5-bis(diphenylphosphino)-9,9-dimethylxanthene (0.54 g, 0.93 mmol, 0.03 eq.) in tetrahydrofuran (50 mL) was added sodium tert-butoxide (0.18 g, 1.87 mmol, 0.06 eq.) at 0° C. The reaction solution was stirred at 0° C. for 1 h. To the above mixture was added 4,4,5,5-tetramethyl-2-(4,4,5,5-tetramethyl-1,3,2-dioxaborolan-2-yl)-1,3,2-dioxaborolane (15.85 g, 62.43 mmol, 2.0 eq.) in portions at room temperature. The resulting mixture was stirred for additional 1 h at room temperature. To the above mixture was added benzyl prop-2-ynoate (5.0 g, 31.22 mmol, 1.0 eq.) and methanol (1.5 g, 46.82 mmol, 1.5 eq.) in portions at room temperature. The resulting mixture was stirred for additional 14 h at 25° C. The aqueous layer was extracted with methylene chloride (3×200 mL). The residue was purified by silica gel chromatography (column height: 250 mm, diameter: 100 mm, 100-200 mesh silica gel, petroleum ether/ethyl acetate=1/0, 3/1). 5.0 g (55%) of desired product was obtained as a colorless liquid.

### Intermediate Example 48

### Synthesis of tert-butyl 4-(2-bromo-5-nitrophenoxy)butanoate

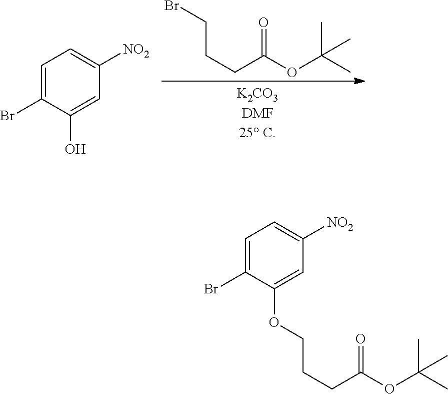 [SMILES: O=[N+]([O-])c1ccc(Br)c(O)c1 ; CC(C)(C)OC(=O)CCCBr ; CC(C)(C)OC(=O)CCCOc1cc([N+](=O)[O-])ccc1Br]

To a stirred solution of 2-bromo-5-nitrophenol (5.0 g, 22.9 mmol, 1.0 eq.) and tert-butyl 4-bromobutanoate (7.7 g, 34.40 mmol, 1.5 eq.) in N,N-dimethylformamide (100.0 mL) was added potassium carbonate (9.5 g, 68.8 mmol, 3 eq.) in portions at 25° C. under nitrogen atmosphere. The resulting mixture was stirred for 3 h at 25° C. under nitrogen atmosphere, diluted with water (500 mL). The resulting mixture was extracted with dichloromethane (3×500 mL) and organic layers were washed with saturated sodium chloride solution (3×500 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 5 g (60%) of tert-butyl 4-(2-bromo-5-nitrophenoxy)butanoate was obtained as a yellow solid.

### Intermediate Example 49

### Synthesis of tert-butyl (E)-4-(2-(3-(benzyloxy)-3-oxoprop-1-en-1-yl)-5-nitrophenoxy)butanoate

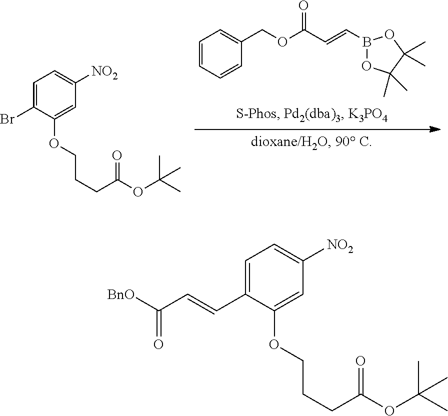 [SMILES: CC(C)(C)OC(=O)CCCOc1cc([N+](=O)[O-])ccc1Br ; CC1(C)OB(/C=C/C(=O)OCc2ccccc2)OC1(C)C ; CC(C)(C)OC(=O)CCCOc1cc([N+](=O)[O-])ccc1/C=C/C(=O)OCc1ccccc1]

To a stirred solution of tert-butyl 4-(2-bromo-5-nitrophenoxy)butanoate (5.0 g, 13.88 mmol, 1.0 eq.), benzyl (2E)-3-(4,4,5,5-tetramethyl-1,3,2-dioxaborolan-2-yl)prop-2-enoate (4.8 g, 16.65 mmol, 1.2 eq.) and potassium phosphate tribasic (5.89 g, 27.76 mmol, 2.0 eq.) in N,N-dimethylformamide (50.0 mL) were added 2-dicyclohexylphosphino-2′,6′-dimethoxy-1,1′-biphenyl (0.28 g, 0.69 mmol, 0.05 eq.) and tris(dibenzylideneacetone)dipalladium (0.64 g, 0.69 mmol, 0.05 eq.) in portions at room temperature under nitrogen atmosphere and the reaction solution was stirred at 90° C. for another 12 hours under nitrogen atmosphere. The resulting mixture was cooled to 25° C., diluted with water (500 mL). The resulting mixture was extracted with dichloromethane (3×500 mL) and organic layers were washed with saturated sodium chloride solution (3×500 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 3 g (49%) of tert-butyl (E)-4-(2-(3-(benzyloxy)-3-oxoprop-1-en-1-yl)-5-nitrophenoxy)butanoate was obtained as a yellow oil.

### Intermediate Example 50

### Synthesis of 3-(4-amino-2-(4-(tert-butoxy)-4-oxobutoxy)phenyl)propanoic acid

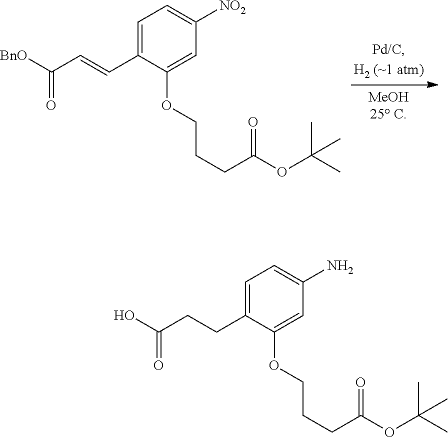 [SMILES: CC(C)(C)OC(=O)CCCOc1cc([N+](=O)[O-])ccc1/C=C/C(=O)OCc1ccccc1 ; CC(C)(C)OC(=O)CCCOc1cc(N)ccc1CCC(=O)O]

To a solution of tert-butyl (E)-4-(2-(3-(benzyloxy)-3-oxoprop-1-en-1-yl)-5-nitrophenoxy)butanoate (3.0 g, 6.80 mmol, 1.0 eq.) in methanol (30.0 mL), was added palladium/carbon (1.0 g). The flask was evacuated and flushed five times with hydrogen. The resulting solution was stirred for 3 h at 25° C. The solids were filtered out and washed with methanol (3×10 mL). The resulting mixture was concentrated under reduced pressure and the crude product without further purification was used at next step directly. 2.5 g (81%) of 3-(4-amino-2-(4-(tert-butoxy)-4-oxobutoxy)phenyl)propanoic acid was obtained as a white solid.

### Intermediate Example 51

### Synthesis of 3-(2-(4-(tert-butoxy)-4-oxobutoxy)-4-(2,5-dioxo-2,5-dihydro-1H-pyrrol-1-yl)phenyl)propanoic acid

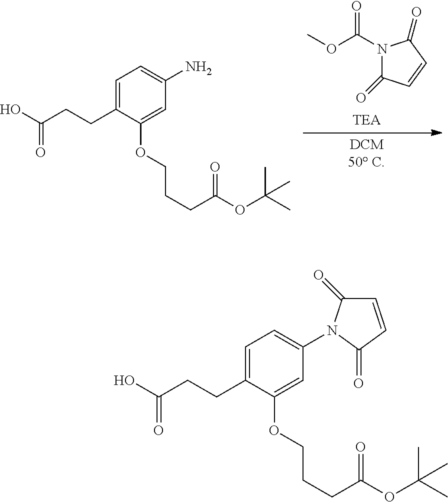 [SMILES: CC(C)(C)OC(=O)CCCOc1cc(N)ccc1CCC(=O)O ; COC(=O)N1C(=O)C=CC1=O ; CC(C)(C)OC(=O)CCCOc1cc(N2C(=O)C=CC2=O)ccc1CCC(=O)O]

To a solution of 3-(4-amino-2-(4-(tert-butoxy)-4-oxobutoxy)phenyl)propanoic acid (2.5 g, 7.73 mmol, 1.0 eq.) and methyl 2,5-dioxopyrrole-1-carboxylate (1.80 g, 11.59 mmol, 1.5 eq.) in dichloromethane (25.0 mL) was added trimethylamine (1.56 g, 15.46 mmol, 2.0 eq.) at room temperature under nitrogen atmosphere. The resulting mixture was stirred for 4 h at 50° C. The resulting mixture was cooled to 25° C. and concentrated under reduced pressure. 1.5 g (48%) of 3-(2-(4-(tert-butoxy)-4-oxobutoxy)-4-(2,5-dioxo-2,5-dihydro-1H-pyrrol-1-yl)phenyl)propanoic acid was obtained as a yellow oil. MS m/z [M−H]⁻ (ESI): 402.16.

### Intermediate Example 52

### Synthesis of tert-butyl 4-(5-(2,5-dioxo-2,5-dihydro-1H-pyrrol-1-yl)-2-(3-oxo-3-(2,3,5,6-tetrafluorophenoxy)propyl)phenoxy)butanoate

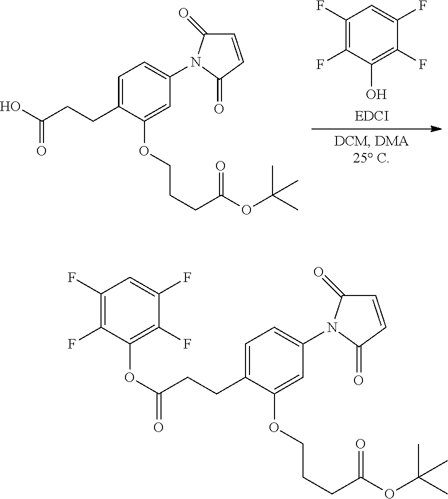 [SMILES: CC(C)(C)OC(=O)CCCOc1cc(N2C(=O)C=CC2=O)ccc1CCC(=O)O ; Oc1c(F)c(F)cc(F)c1F ; CC(C)(C)OC(=O)CCCOc1cc(N2C(=O)C=CC2=O)ccc1CCC(=O)Oc1c(F)c(F)cc(F)c1F]

A solution of 3-(2-(4-(tert-butoxy)-4-oxobutoxy)-4-(2,5-dioxo-2,5-dihydro-1H-pyrrol-1-yl)phenyl)propanoic acid (1.5 g, 3.71 mmol, 1 eq.) in N,N-dimethylformamide (15 mL) was treated with 2,3,5,6-tetrafluor-phenol (1.85 g, 11.15 mmol, 3 eq.) at room temperature under nitrogen atmosphere followed by the addition of 1-ethyl-3(3-dimethylpropylamine) carbodiimide (2.14 g, 11.15 mmol, 3 eq.) at 25° C. The resulting mixture was stirred for 2 h at 25° C. under nitrogen atmosphere. The residue was purified by reversed-phase flash chromatography with the following conditions: column, C₁₈ silica gel; mobile phase, acetonitrile in water (0.1% formic acid), 10% to 100% gradient in 10 min; detector, UV 254 nm. The resulting mixture was concentrated under reduced pressure. 800 mg (39%) of tert-butyl 4-(5-(2,5-dioxo-2,5-dihydro-1H-pyrrol-1-yl)-2-(3-oxo-3-(2,3,5,6-tetrafluorophenoxy)propyl)phenoxy)butanoate was obtained as a colorless oil.

### Intermediate Example 53

### Synthesis of 4-(5-(2,5-dioxo-2,5-dihydro-1H-pyrrol-1-yl)-2-(3-oxo-3-(2,3,5,6-tetrafluorophenoxy)propyl)phenoxy)butanoic acid

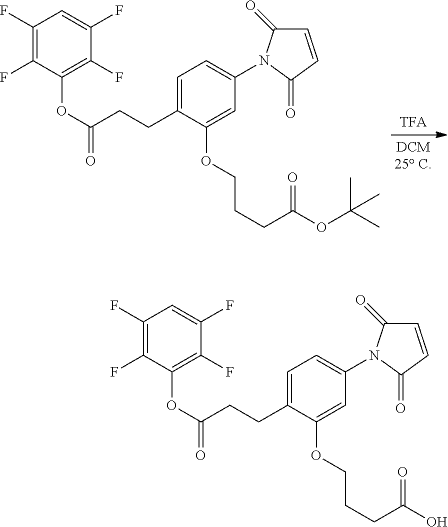 [SMILES: CC(C)(C)OC(=O)CCCOc1cc(N2C(=O)C=CC2=O)ccc1CCC(=O)Oc1c(F)c(F)cc(F)c1F ; O=C(O)CCCOc1cc(N2C(=O)C=CC2=O)ccc1CCC(=O)Oc1c(F)c(F)cc(F)c1F]

A solution of tert-butyl 4-(5-(2,5-dioxo-2,5-dihydro-1H-pyrrol-1-yl)-2-(3-oxo-3-(2,3,5,6-tetrafluorophenoxy)propyl)phenoxy)butanoate (800 mg, 1.45 mmol, 1 eq.) in dichloromethane (6.0 mL) with an inert atmosphere of nitrogen, was added trifluoroacetic acid (2.0 mL) at 25° C. The resulting mixture was stirred for 2 h at 25° C. The resulting mixture was concentrated under reduced pressure. 700 mg (97%) of 4-(5-(2,5-dioxo-2,5-dihydro-1H-pyrrol-1-yl)-2-(3-oxo-3-(2,3,5,6-tetrafluorophenoxy)propyl)phenoxy)butanoic acid was obtained as a yellow oil.

### Intermediate Example 54

### Synthesis of (2,3,5,6-tetrafluorophenyl) 3-[2-chlorocarbonyl-4-(2,5-dioxopyrrol-1-yl)phenyl]propanoate

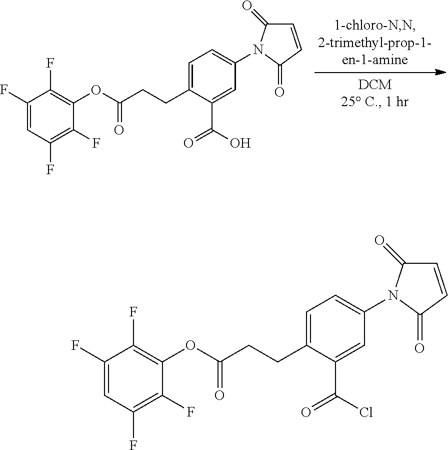 [SMILES: O=C(CCc1ccc(N2C(=O)C=CC2=O)cc1C(=O)O)Oc1c(F)c(F)cc(F)c1F ; O=C(CCc1ccc(N2C(=O)C=CC2=O)cc1C(=O)Cl)Oc1c(F)c(F)cc(F)c1F]

To a solution of 5-(2,5-dioxopyrrol-1-yl)-2-[3-oxo-3-(2,3,5,6-tetrafluorophenoxy)propyl] benzoic acid (1.80 g, 4.12 mmol, 1.0 eq) in DCM (36 mL) was added 1-chloro-N,N,2-trimethyl-prop-1-en-1-amine (1.10 g, 8.23 mmol, 1.09 mL, 2.0 eq). The mixture was stirred at 25° C. for 1 hr. The reaction mixture was concentrated under reduced pressure to remove solvent. Used for next step directly with no further purification. Compound (2,3,5,6-tetrafluorophenyl) 3-[2-chlorocarbonyl-4-(2,5-dioxopyrrol-1-yl)phenyl]propanoate (1.88 g, 4.13 mmol, 100% yield) was obtained as yellow oil.

### Intermediate Example 55

### Synthesis of 2-[[2-[[2-[[2-[[2-[[2-[[2-[[2-[[2-[[2-[[5-(2,5-dioxopyrrol-1-yl)-2-[3-oxo-3-(2,3,5,6-tetrafluorophenoxy)propyl]benzoyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetic acid

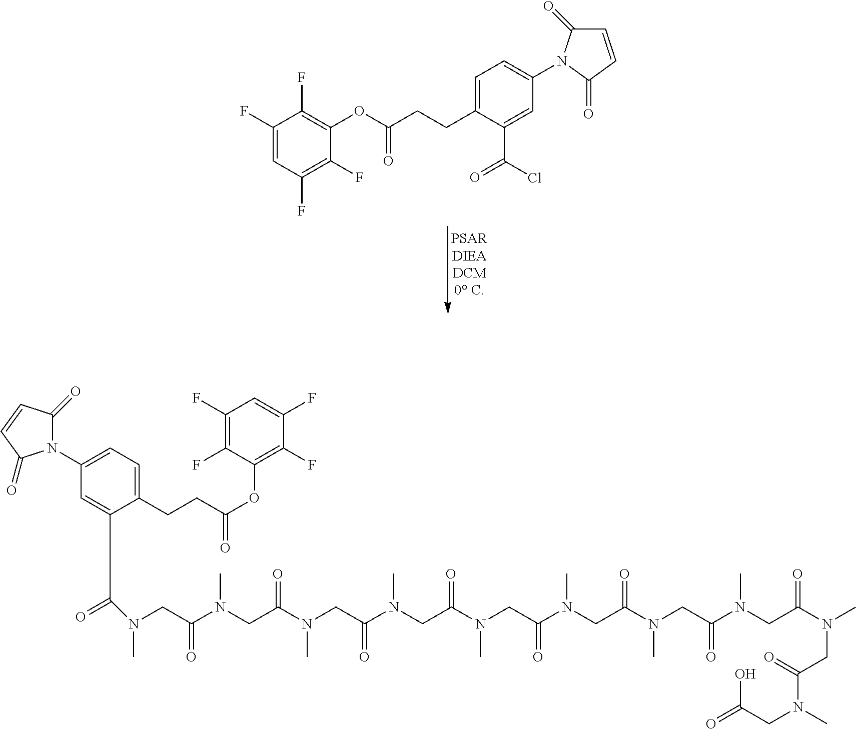 [SMILES: O=C(CCc1ccc(N2C(=O)C=CC2=O)cc1C(=O)Cl)Oc1c(F)c(F)cc(F)c1F ; CN(CC(=O)O)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)c1cc(N2C(=O)C=CC2=O)ccc1CCC(=O)Oc1c(F)c(F)cc(F)c1F]

To a solution of 2-[methyl-[2-[methyl-[2-[methyl-[2-[methyl-[2-[methyl-[2-[methyl-[2-[methyl-[2-[methyl-[2-[methyl-[2-(methylamino)acetyl]amino]acetyl]amino]acetyl]amino]-acetyl]amino]acetyl]amino]acetyl]amino]acetyl]amino]acetyl]amino]acetyl]amino]acetic acid (3.01 g, 4.13 mmol, 1.0 eq) PSAR in DCM (36 mL) was added DIEA (1.60 g, 12.4 mmol, 2.16 mL, 3.0 eq) at 0° C. After 10 min, to a solution of (2,3,5,6-tetrafluorophenyl) 3-[2-chlorocarbonyl-4-(2,5-dioxopyrrol-1-yl)phenyl]propanoate (1.88 g, 4.13 mmol, 1.0 eq) in DCM (18 mL) was added, and then stirred at 0° C. for 1 hr. The pH of the resulting mixture was adjusted to ˜6 with TFA at 0° C., and diluted with addition MeCN (2 mL) and concentrated under reduced pressure to remove DCM. The residue was purified by prep-HPLC (column: Phenomenex Luna C₁₈ (250×70 mm, 15 μm); mobile phase: [water (TFA)-ACN]; B %: 25%-55%, 20 min). 2-[[2-[[2-[[2-[[2-[[2-[[2-[[2-[[2-[[2-[[5-(2,5-dioxopyrrol-1-yl)-2-[3-oxo-3-(2,3,5,6-tetrafluorophenoxy)propyl]benzoyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetic acid (360 mg, 314 μmol, 7.60% yield) was obtained as a white solid: LC/MS [M+H] 1148.4 (calculated); LC/MS [M+H] 1148.5 (observed).

### Intermediate Example 56

### Synthesis of (2S,3S,4S,5R,6S)-2-(methoxycarbonyl)-6-(2-nitro-4-((((4-nitrophenoxy)carbonyl)oxy)methyl)phenoxy)tetrahydro-2H-pyran-3,4,5-triyl triacetate

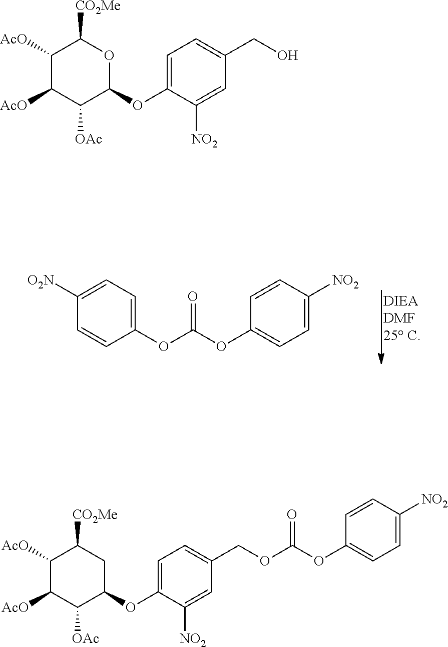 [SMILES: COC(=O)[C@H]1O[C@@H](Oc2ccc(CO)cc2[N+](=O)[O-])[C@H](OC(C)=O)[C@@H](OC(C)=O)[C@@H]1OC(C)=O ; O=C(Oc1ccc([N+](=O)[O-])cc1)Oc1ccc([N+](=O)[O-])cc1 ; COC(=O)[C@H]1C[C@@H](Oc2ccc(COC(=O)Oc3ccc([N+](=O)[O-])cc3)cc2[N+](=O)[O-])[C@H](OC(C)=O)[C@@H](OC(C)=O)[C@@H]1OC(C)=O]

To a stirred solution of methyl (2S,3S,4S,5R,6S)-3,4,5-tris(acetyloxy)-6-[4-(hydroxymethyl)-2-nitrophenoxy]oxane-2-carboxylate (5.0 g, 10.3 mmol, 1.0 eq.) and diisopropylethylamine (4.0 g, 31.0 mmol, 3.0 eq.) in N,N-Dimethylformamide (50.0 mL) was added bis(4-nitrophenyl) carbonate (4.7 g, 15.5 mmol, 1.5 eq.) at room temperature under air atmosphere. The resulting solution was stirred for 16 h at 25° C., diluted with water (500 mL). The resulting mixture was extracted with dichloromethane (3×500 mL) and organic layers were washed with saturated sodium chloride solution (3×500 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 5.5 g (82%) of (2S,3S,4S,5R,6S)-2-(methoxycarbonyl)-6-(2-nitro-4-((((4-nitrophenoxy)carbonyl)oxy)methyl)phenoxy)tetrahydro-2H-pyran-3,4,5-triyl triacetate was obtained as a white solid. MS m/z [M+NH₄]⁺ (ESI): 668.12.

### Intermediate Example 57

### Synthesis of (2S,3R,4S,5S,6S)-2-(4-(((ethylcarbamoyl)oxy)methyl)-2-nitrophenoxy)-6-(methoxycarbonyl)tetrahydro-2H-pyran-3,4,5-triyl triacetate

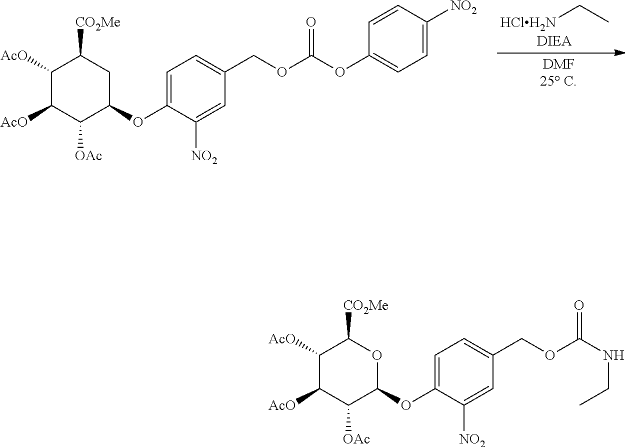

To a stirred solution of (2S,3S,4S,5R,6S)-2-(methoxycarbonyl)-6-(2-nitro-4-((((4-nitrophenoxy)carbonyl)oxy)methyl)phenoxy)tetrahydro-2H-pyran-3,4,5-triyl triacetate (5.5 g, 8.5 mmol, 1.0 eq.) and ethylamine (1.4 g, 16.9 mmol, 2.0 eq.) in N,N-dimethylformamide (50.0 mL) was added diisopropylethylamin (3.3 g, 25.4 mmol, 3.0 eq.) at 25° C. under air atmosphere. The resulting solution was stirred for 2 h at 25° C., diluted with water (500 mL). The resulting mixture was extracted with dichloromethane (3×500 mL) and organic layers were washed with saturated sodium chloride solution (3×500 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 3.5 g (75%) of (2S,3R,4S,5S,6S)-2-(4-(((ethylcarbamoyl)oxy)methyl)-2-nitrophenoxy)-6-(methoxycarbonyl)tetrahydro-2H-pyran-3,4,5-triyl triacetate was obtained as a white solid. MS m/z [M+NH₄]⁺ (ESI): 574.15.

### Intermediate Example 58

### Synthesis of (2S,3R,4S,5S,6S)-2-(4-((((chloromethyl)(ethyl)carbamoyl)oxy)methyl)-2-nitrophenoxy)-6-(methoxycarbonyl)tetrahydro-2H-pyran-3,4,5-triyl triacetate

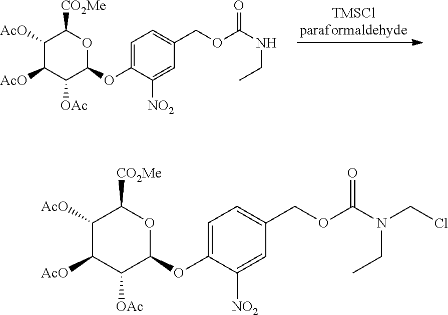 [SMILES: CCNC(=O)OCc1ccc(O[C@@H]2O[C@H](C(=O)OC)[C@@H](OC(C)=O)[C@H](OC(C)=O)[C@H]2OC(C)=O)c([N+](=O)[O-])c1 ; CCN(CCl)C(=O)OCc1ccc(O[C@@H]2O[C@H](C(=O)OC)[C@@H](OC(C)=O)[C@H](OC(C)=O)[C@H]2OC(C)=O)c([N+](=O)[O-])c1]

To a stirred solution of (2S,3R,4S,5S,6S)-2-(4-(((ethylcarbamoyl)oxy)methyl)-2-nitrophenoxy)-6-(methoxycarbonyl)tetrahydro-2H-pyran-3,4,5-triyl triacetate (3.5 g, 6.3 mmol, 1.0 eq.) and paraformaldehyde (1.7 g, 18.9 mmol, 3.0 eq.) in dichloromethane (35.0 mL) was added chlorotrimethylsilane (2.1 g, 18.9 mmol, 3.0 eq.) at 25° C. under air atmosphere. The resulting mixture was stirred for additional 2 h at 25° C. The resulting mixture was concentrated under reduced pressure to afford (2S,3R,4S,5S,6S)-2-(4-((((chloromethyl)(ethyl)carbamoyl)oxy)methyl)-2-nitrophenoxy)-6-(methoxycarbonyl)tetrahydro-2H-pyran-3,4,5-triyl triacetate (3.0 g, 79%) as a white solid.

### Intermediate Example 59

### Synthesis of (2S,3R,4S,5S,6S)-2-(4-(((((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)(ethyl)carbamoyl)oxy)methyl)-2-nitrophenoxy)-6-(methoxycarbonyl)tetrahydro-2H-pyran-3,4,5-triyl triacetate

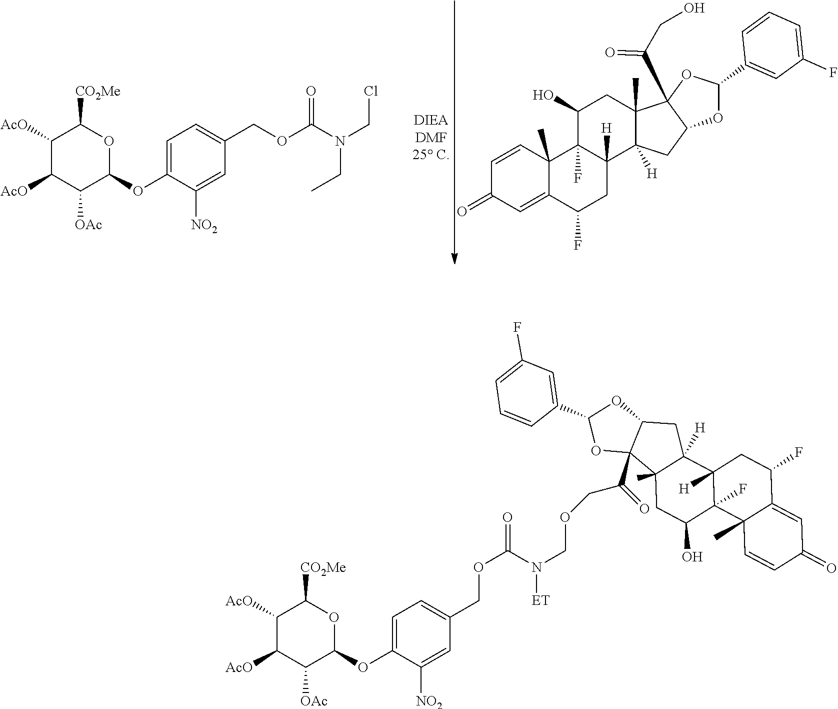

To a stirred solution of (2S,3R,4S,5S,6S)-2-(4-((((chloromethyl)(ethyl)carbamoyl)oxy)methyl)-2-nitrophenoxy)-6-(methoxycarbonyl)tetrahydro-2H-pyran-3,4,5-triyl triacetate (3.0 g, 5.0 mmol, 2 eq.) and diisopropylethylamine (51.28 mg, 0.397 mmol, 3 eq.) in N,N-dimethylformamide (20.0 mL) was added (2S,6aS,6bR,7S,8aS,8bS,10R,11aR,12aS,12bS)-2,6b-difluoro-10-(3-fluorophenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (1.3 g, 2.5 mmol, 1.0 eq.) at 25° C. under air atmosphere. The resulting solution was stirred for 2 h at 25° C., diluted with water (300 mL). The resulting mixture was extracted with dichloromethane (3×300 mL) and organic layers were washed with saturated sodium chloride solution (3×300 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 1.1 g (41%) of (2S,3R,4S,5S,6S)-2-(4-(((((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)(ethyl)carbamoyl)oxy)methyl)-2-nitrophenoxy)-6-(methoxycarbonyl)tetrahydro-2H-pyran-3,4,5-triyl triacetate was obtained as a white solid. MS m/z [M+H]⁺ (ESI): 1087.35.

### Intermediate Example 60

### Synthesis of (2S,3R,4S,5S,6S)-2-(2-amino-4-(((((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)(ethyl)carbamoyl)oxy)methyl)phenoxy)-6-(methoxycarbonyl)tetrahydro-2H-pyran-3,4,5-triyl triacetate

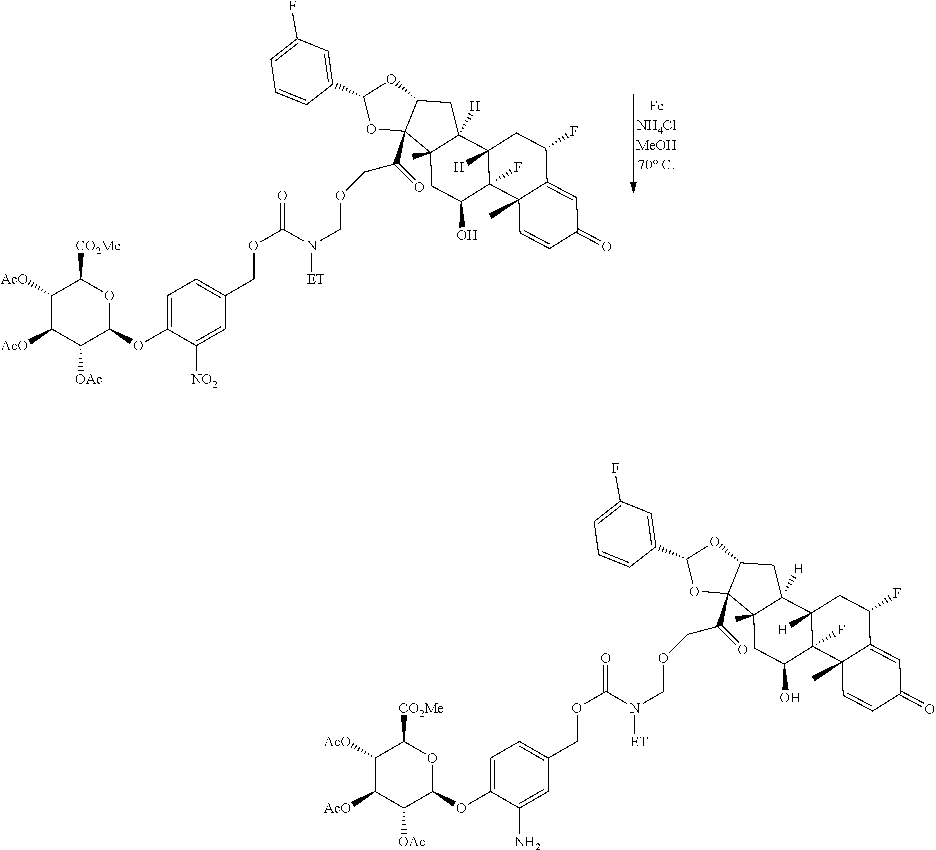

To a stirred solution methyl (2S,3R,4S,5S,6S)-2-(4-(((((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)(ethyl)carbamoyl)oxy)methyl)-2-nitrophenoxy)-6-(methoxycarbonyl)tetrahydro-2H-pyran-3,4,5-triyl triacetate (1.1 g, 1.0 mmol, 1.0 eq.) and ammonium chloride (536.8 mg, 10.1 mmol, 10.0 eq.) in methanol (11.0 mL) was added iron powder (1.1 g, 20.0 mmol, 20.0 eq.) at 25° C. The resulting mixture was stirred for 5 h at 70° C. The solids were filtered out and washed with methanol (3×10 mL). The resulting mixture was concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 810 mg (76%) of (2S,3R,4S,5S,6S)-2-(2-amino-4-(((((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)(ethyl)carbamoyl)oxy)methyl)phenoxy)-6-(methoxycarbonyl)tetrahydro-2H-pyran-3,4,5-triyl triacetate was obtained as a white solid. MS m/z [M+H]⁺ (ESI): 1057.37.

### Intermediate Example 61

### Synthesis of (2S,3R,4S,5S,6S)-2-(2-(3-((((9H-fluoren-9-yl)methoxy)carbonyl)amino)propanamido)-4-(((((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)(ethyl)carbamoyl)oxy)methyl)phenoxy)-6-(methoxycarbonyl)tetrahydro-2H-pyran-3,4,5-triyl triacetate

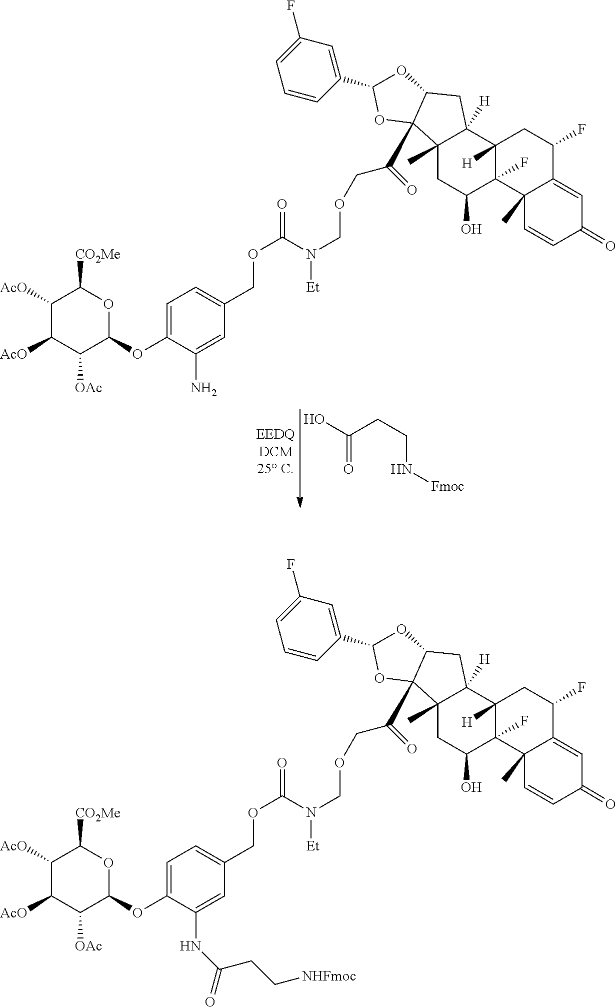

To a stirred solution (2S,3R,4S,5S,6S)-2-(2-amino-4-(((((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)(ethyl)carbamoyl)oxy)methyl)phenoxy)-6-(methoxycarbonyl)tetrahydro-2H-pyran-3,4,5-triyl triacetate (810.0 mg, 0.7 mmol, 1.0 eq.) and ethyl 2-ethoxy-2H-quinoline-1-carboxylate (378.9 mg, 1.5 mmol, 2.0 eq.) in dichloromethane (10.0 mL) was added 3-{[(9H-fluoren-9-ylmethoxy)carbonyl]amino}propanoic acid (357.8 mg, 1.2 mmol, 1.5 eq.) at 25° C. The resulting solution was stirred for 3 h at 25° C., diluted with water (200 mL). The resulting mixture was extracted with dichloromethane (3×300 mL) and organic layers were washed with saturated sodium chloride solution (3×300 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water (10 mmol/L ammonium bicarbonate) and acetonitrile (10.0% acetonitrile up to 100.0% in 20 min); Detector, UV 254 nm. 600.0 mg (58%) of (2S,3R,4S,5S,6S)-2-(2-(3-((((9H-fluoren-9-yl)methoxy)carbonyl)amino)propanamido)-4-(((((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)(ethyl)carbamoyl)oxy)methyl)phenoxy)-6-(methoxycarbonyl)tetrahydro-2H-pyran-3,4,5-triyl triacetate was obtained as a white solid. MS m/z [M+H]⁺ (ESI): 1050.48.

### Intermediate Example 62

### Synthesis of (2S,3S,4S,5R,6S)-6-(2-(3-aminopropanamido)-4-(((((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)(ethyl)carbamoyl)oxy)methyl)phenoxy)-3,4,5-trihydroxytetrahydro-2H-pyran-2-carboxylic acid

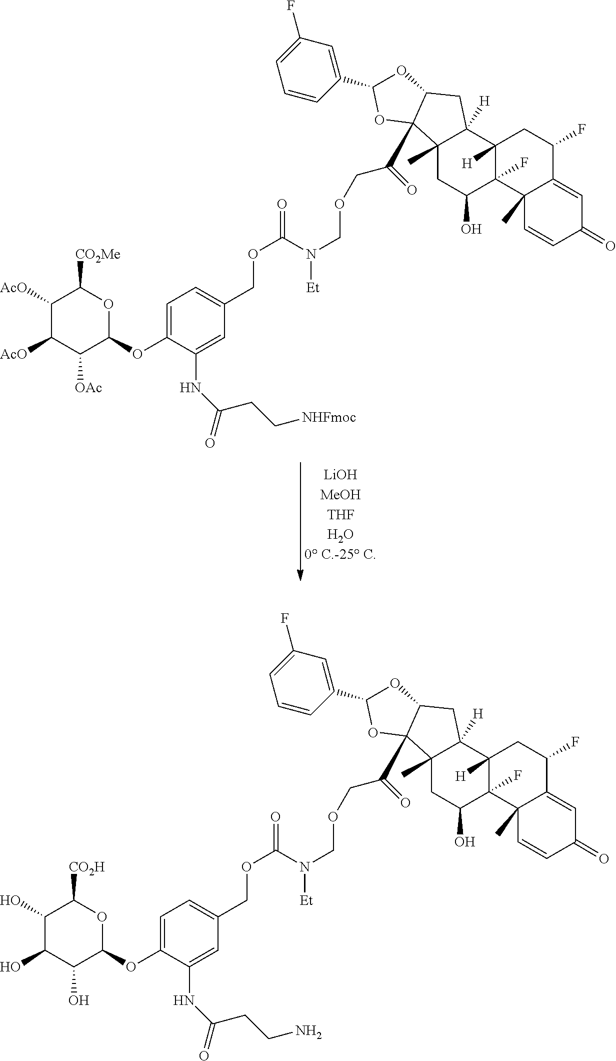

To a stirred solution of (2S,3R,4S,5S,6S)-2-(2-(3-((((9H-fluoren-9-yl)methoxy)carbonyl)amino)propanamido)-4-(((((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)(ethyl)carbamoyl)oxy)methyl)phenoxy)-6-(methoxycarbonyl)tetrahydro-2H-pyran-3,4,5-triyl triacetate (600.0 mg, 0.4 mmol, 1.0 eq.) in methanol (10.0 mL) and water (5.0 mL) with an inert atmosphere of nitrogen, was added lithium hydroxide (31.9 mg, 1.2 mmol, 3.0 eq.) at 0° C. The resulting solution was stirred for 12 h at 25° C., adjusted pH to 7 with hydrochloric acid (1 mol/L). The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 180 mg (41%) of (2S,3S,4S,5R,6S)-6-(2-(3-aminopropanamido)-4-(((((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)(ethyl)carbamoyl)oxy)methyl)phenoxy)-3,4,5-trihydroxytetrahydro-2H-pyran-2-carboxylic acid was obtained as a white solid. MS m/z [M+H]⁺ (ESI): 988.36.

### Intermediate Example 63

### Synthesis of methyl (E)-3-(4,4,5,5-tetramethyl-1,3,2-dioxaborolan-2-yl)acrylate

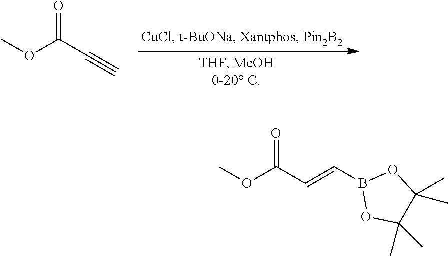

To a solution of copper(I) chloride (1.06 g, 10.7 mmol, 0.03 eq.) and 4,5-Bis(diphenylphosphino)-9,9-dimethylxanthene (6.19 g, 10.7 mmol, 0.03 eq.) in tetrahydrofuran (450.0 mL) was added sodium tert-butoxide (2.06 g, 21.4 mmol, 0.06 eq.) at 0° C. The reaction solution was stirred at 0° C. for 1 hour, followed by addition of a solution of bis(pinacolato)diboron (90.6 g, 357 mmol, 1.0 eq.) in tetrahydrofuran (150.0 mL). The reaction solution was stirred under nitrogen atmosphere at 20° C. for one hour. Methyl prop-2-ynoate (30.0 g, 357.0 mmol, 1.0 eq.) and methanol (22.9 g, 714 mmol, 2.0 eq.) were added to the above reaction solution. And the reaction solution was stirred at 20° C. for another 12 h. The result mixture was poured into ice-water (w/w=1/1) (300 mL) and stirred for 5 min. The aqueous phase was extracted with ethyl acetate (200 mL×3). The combined organic phase was washed with brine (50 mL×2), dried with anhydrous sodium sulfate, filtered and concentrated in vacuum. The residue was purified by silica gel chromatography (column height: 250 mm, diameter: 100 mm, 100-200 mesh silica gel, petroleum ether/ethyl acetate=1/0, 3/1) to afford methyl (E)-3-(4,4,5,5-tetramethyl-1,3,2-dioxaborolan-2-yl)acrylate (70 g, 92%) as colorless oil: ¹H NMR (400 MHz, CDCl₃) δ 6.83-6.75 (m, 1H), 6.68-6.59 (m, 1H), 3.77 (s, 3H), 1.29 (s, 12H).

### Intermediate Example 64

### Synthesis of tert-butyl ((benzyloxy)carbonyl)glycylglycinate

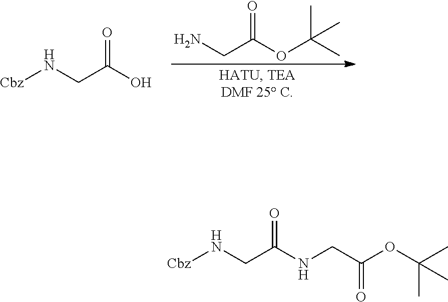

To a solution of 2-(benzyloxycarbonylamino) acetic acid (5.50 g, 26.3 mmol, 1.0 eq.) in N,N-dimethylformamide (100.0 mL) was added triethylamine (7.98 g, 78.9 mmol, 3.0 eq.), 2-(7-Azabenzotriazol-1-yl)-N,N,N′,N′-tetramethyluronium hexafluorophosphate (12.0 g, 31.5 mmol, 1.2 eq.) and tert-butyl 2-aminoacetate (3.45 g, 26.3 mmol, 1.0 eq.), and then stirred at 25° C. for 2 h. The reaction mixture was quenched by addition water (150 mL) at 0° C., and then extracted with ethyl acetate (100 mL×3). The combined organic layers were washed with water (80 mL×3), then were washed with brine (100 mL), dried over sodium sulfate, filtered and concentrated under reduced pressure to give a residue. The residue was purified by silica gel chromatography (column height: 250 mm, diameter: 100 mm, 100-200 mesh silica gel, ethyl acetate/petroleum ether=1/1) to give tert-butyl ((benzyloxy)carbonyl)glycylglycinate (7.7 g, 91%) was obtained as a yellow oil: ¹H NMR (CDCl₃, 400 MHz) δ7.40-7.31 (m, 5H), 5.14 (s, 2H), 3.98-3.89 (m, 4H), 1.47 (s, 9H).

### Intermediate Example 65

### Synthesis of

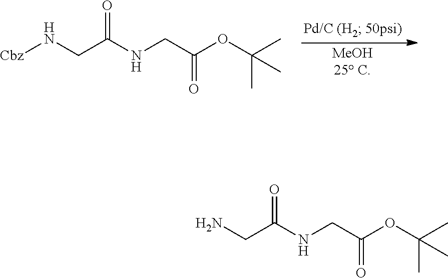 [SMILES: CC(C)(C)OC(=O)CNC(=O)CNC(=O)OCc1ccccc1 ; CC(C)(C)OC(=O)CNC(=O)CN]

To a solution of tert-butyl ((benzyloxy)carbonyl)glycylglycinate (8.8 g, 27.3 mmol, 1.0 eq.) in methanol (150 mL) was added palladium/carbon (10%, 2.5 g) under nitrogen atmosphere. The suspension was degassed and purged with hydrogen for 3 times. The mixture was stirred under hydrogen (50 Psi) at 25° C. for 2 h. The reaction mixture was filtered and concentrated under reduced pressure to give a residue. tert-butyl glycylglycinate (4.2 g, 81%) was obtained as a white solid. MS m/z [M+H]⁺ (ESI): 189.12; ¹H NMR (CDCl₃, 400 MHz) δ 7.67 (s, 1H), 3.96 (d, J=5.6 Hz, 2H), 3.39 (s, 2H), 1.75 (s, 2H), 1.46 (s, 9H).

### Intermediate Example 66

### Synthesis of tert-butyl 2-bromo-5-nitrobenzoate

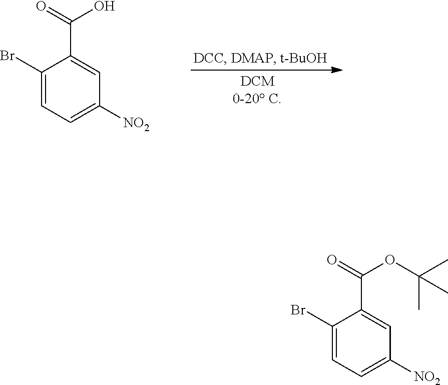 [SMILES: O=C(O)c1cc([N+](=O)[O-])ccc1Br ; CC(C)(C)OC(=O)c1cc([N+](=O)[O-])ccc1Br]

To a mixture of 2-bromo-5-nitro-benzoic acid (9.7 g, 39.4 mmol, 1.0 eq.) in dichloromethane (100 mL) was added dicyclohexylcarbodiimide (8.95 g, 43.4 mmol, 1.1 eq.) and 4-dimethylaminopyridine (2.41 g, 19.7 mmol, 0.5 eq.) at 0° C. The mixture was stirred at 0° C. for 10 min, then tert-butanol (4.4 g, 59.1 mmol, 1.5 eq.) was added and stirred at 20° C. for 12 h. The mixture was poured into ice-water (w/w=1/1) (50 mL) and stirred for 10 min. The aqueous phase was extracted with dichloromethane (500 mL×3). The combined organic phase was washed with brine (100 mL×3), dried with anhydrous sodium sulfate, filtered and concentrated in vacuum. The residue was purified by silica gel chromatography (column height: 250 mm, diameter: 100 mm, 100-200 mesh silica gel, petroleum ether/ethyl acetate=1/0, 5/1) to afford tert-butyl 2-bromo-5-nitrobenzoate (9.2 g, 77%) as white solid: ¹H NMR (400 MHz, CDCl₃) δ 8.52 (d, J=2.8 Hz, 1H), 8.13 (dd, J=2.8, 8.8 Hz, 1H), 7.83 (d, J=8.8 Hz, 1H), 1.65 (s, 9H).

### Intermediate Example 67

### Synthesis of tert-butyl (E)-2-(3-methoxy-3-oxoprop-1-en-1-yl)-5-nitrobenzoate

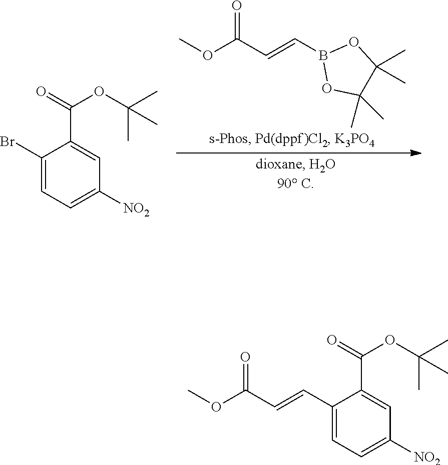

A mixture of methyl (E)-3-(4,4,5,5-tetramethyl-1,3,2-dioxaborolan-2-yl)acrylate (11.2 g, 53.0 mmol, 2.5 eq.), tert-butyl 2-bromo-5-nitrobenzoate (6.40 g, 21.2 mmol, 1.0 eq.), potassium phosphate tribasic (6.74 g, 31.8 mmol, 1.5 eq.), 2-dicyclohexylphosphino-2′,6′-dimethoxy-1,1′-biphenyl (870 mg, 2.12 mmol, 0.1 eq.), tris(dibenzylideneacetone)dipalladium (970 mg, 1.06 mmol, 0.05 eq.) in dioxane (120.0 mL) and water (25.0 mL) was degassed and purged with nitrogen for 3 times, and then the mixture was stirred at 90° C. for 12 h under nitrogen atmosphere. The mixture was poured into ice-water (w/w=1/1) (30 mL) and stirred for 10 min. The aqueous phase was extracted with ethyl acetate (500 mL×3). The combined organic phase was washed with brine (100 mL×3), dried with anhydrous sodium sulfate, filtered and concentrated in vacuum. The residue was purified by silica gel chromatography (column height: 250 mm, diameter: 100 mm, 100-200 mesh silica gel, petroleum ether/ethyl acetate=1/0, 3/1) obtained tert-butyl (E)-2-(3-methoxy-3-oxoprop-1-en-1-yl)-5-nitrobenzoate (6 g, 92%) as yellow solid: ¹H NMR (400 MHz, CDCl₃) δ 8.74 (d, J=2.4 Hz, 1H), 8.41 (d, J=15.6 Hz, 1H), 8.34 (dd, J=2.4, 8.4 Hz, 1H), 7.72 (d, J=8.4 Hz, 1H), 6.38 (d, J=15.6 Hz, 1H), 3.85 (s, 3H), 1.65 (s, 9H).

### Intermediate Example 68

### Synthesis of tert-butyl 5-amino-2-(3-methoxy-3-oxopropyl)benzoate

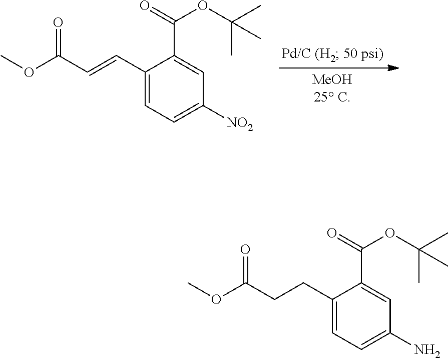 [SMILES: COC(=O)/C=C/c1ccc([N+](=O)[O-])cc1C(=O)OC(C)(C)C ; COC(=O)CCc1ccc(N)cc1C(=O)OC(C)(C)C]

A mixture of tert-butyl (E)-2-(3-methoxy-3-oxoprop-1-en-1-yl)-5-nitrobenzoate (4.0 g, 13.0 mmol, 1.0 eq.), palladium/carbon (10%, 400.0 mg) in methanol (50.0 mL) was degassed and purged with hydrogen for 3 times, and then the mixture was stirred at 25° C. for 3 h under hydrogen atmosphere. The mixture was filtered and concentrated in vacuum to afford tert-butyl 5-amino-2-(3-methoxy-3-oxopropyl)benzoate (3.50 g, 96%) as yellow oil: [M+H]⁺ (ESI): 280.30; TH NMR (400 MHz, CDCl₃) δ 7.14 (d, J=2.4 Hz, 1H), 7.04 (d, J=8.0 Hz, 1H), 6.73 (dd, J=2.4, 8.0 Hz, 1H), 3.67 (s, 3H), 3.12 (t, J=8.0 Hz, 2H), 2.61 (t, J=8.0 Hz, 2H), 1.59 (s, 9H).

### Intermediate Example 69

### Synthesis of 5-amino-2-(3-methoxy-3-oxopropyl)benzoic acid

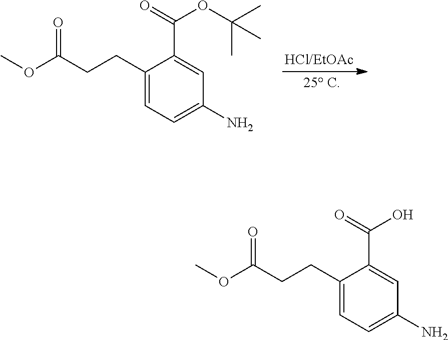

To a solution of tert-butyl 5-amino-2-(3-methoxy-3-oxopropyl)benzoate (3.5 g, 12.5 mmol, 1.0 eq.) in ethyl acetate (10.0 mL) was added hydrochloride (gas)/ethyl acetate (4 mol/L, 50 mL, 16.0 eq.), and then stirred at 25° C. for 12 hours. The mixture was concentrated in vacuum to afford 5-amino-2-(3-methoxy-3-oxopropyl)benzoic acid (3.20 g, 98%) as white solid: [M+H]⁺ (ESI): 224.23; TH NMR (400 MHz, DMSO-d₆) δ 7.69 (d, J=2.0 Hz, 1H), 7.39-7.31 (m, 2H), 3.57 (s, 3H), 3.15 (t, J=7.6 Hz, 2H), 2.59 (t, J=7.6 Hz, 2H).

### Intermediate Example 70

### Synthesis of methyl 3-(4-amino-2-((2-((2-(tert-butoxy)-2-oxoethyl)amino)-2-oxoethyl)carbamoyl)phenyl)propanoate

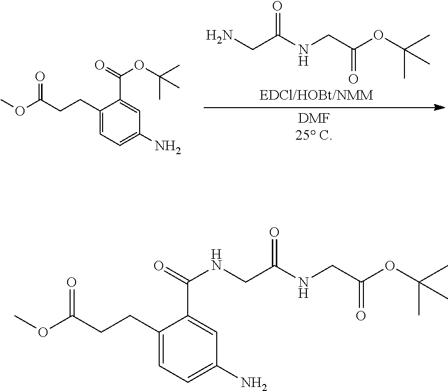 [SMILES: COC(=O)CCc1ccc(N)cc1C(=O)OC(C)(C)C ; CC(C)(C)OC(=O)CNC(=O)CN ; COC(=O)CCc1ccc(N)cc1C(=O)NCC(=O)NCC(=O)OC(C)(C)C]

To a solution of 5-amino-2-(3-methoxy-3-oxopropyl)benzoic acid (3.20 g, 12.3 mmol, 1.0 eq.) in N,N-dimethylformamide (40 mL) was added 4-methylmorpholine (3.74 g, 37.0 mmol, 3.0 eq.), 1-hydroxybenzotriazole (833 mg, 6.16 mmol, 0.5 eq.), N-(3-dimethylaminopropyl)-N′-ethylcarbodiimide hydrochloride (4.72 g, 24.7 mmol, 2.0 eq.) and tert-butyl 2-[(2-aminoacetyl)amino]acetate (2.78 g, 14.8 mmol, 1.2 eq.) at 0° C., and then stirred at 20° C. for 2 h. The mixture was poured into ice-water (w/w=1/1) (40 mL) and stirred for 10 min. The aqueous phase was extracted with ethyl acetate (500 mL×3). The combined organic phase was washed with brine (200 mL×3), dried with anhydrous sodium sulfate, filtered and concentrated in vacuum. The residue was purified by silica gel chromatography (column height: 250 mm, diameter: 100 mm, 100-200 mesh silica gel, petroleum ether/ethyl acetate=1/0, 0/1) to afford methyl 3-(4-amino-2-((2-((2-(tert-butoxy)-2-oxoethyl)amino)-2-oxoethyl)carbamoyl)phenyl)propanoate (3.8 g, 78%) as yellow oil: [M+H]⁺ (ESI): 394.35; ¹H NMR (400 MHz, CDCl₃) δ 7.04 (d, J=8.0 Hz, 2H), 6.79-6.66 (m, 3H), 4.15 (d, J=5.6 Hz, 2H), 3.97 (d, J=5.2 Hz, 2H), 3.61 (s, 3H), 2.99 (t, J=7.2 Hz, 2H), 2.68 (t, J=7.2 Hz, 2H), 1.47 (s, 9H).

### Intermediate Example 71

### Synthesis of (5-amino-2-(3-methoxy-3-oxopropyl)benzoyl)glycylglycine

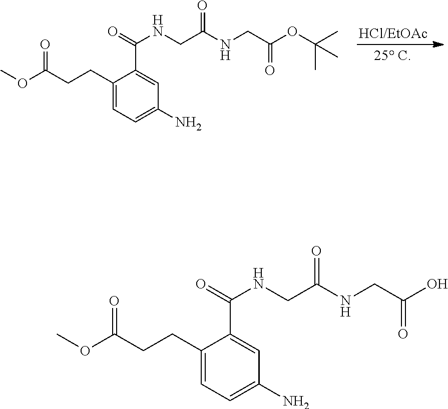

To a solution of methyl 3-(4-amino-2-((2-((2-(tert-butoxy)-2-oxoethyl)amino)-2-oxoethyl)carbamoyl)phenyl)propanoate (0.60 g, 1.53 mmol, 1.0 eq.) in ethyl acetate (5.0 mL) was added hydrochloride (gas)/ethyl acetate (4 mol/L, 10 mL, 26.2 eq.), and then stirred at 25° C. for 12 h. The mixture was concentrated in vacuum to afford (5-amino-2-(3-methoxy-3-oxopropyl)benzoyl)glycylglycine (550 mg, 96% yield) as white solid: [M+H]⁺ (ESI): 338.14; ¹H NMR (400 MHz, DMSO-d₆) δ 8.57 (t, J=6.0 Hz, 1H), 8.25 (t, J=6.4 Hz, 1H), 7.28 (d, J=8.0 Hz, 1H), 7.20-7.10 (m, 2H), 3.89 (d, J=6.0 Hz, 2H), 3.81 (d, J=6.0 Hz, 2H), 3.57 (s, 3H), 2.90 (t, J=8.0 Hz, 2H), 2.60 (t, J=8.0 Hz, 2H).

### Intermediate Example 72

### Synthesis of tert-butyl 1-(5-amino-2-(3-methoxy-3-oxopropyl)phenyl)-1,4,7-trioxo-11,14,17,20,23,26,29,32,35,38,41,44-dodecaoxa-2,5,8-triazaheptatetracontan-47-oate

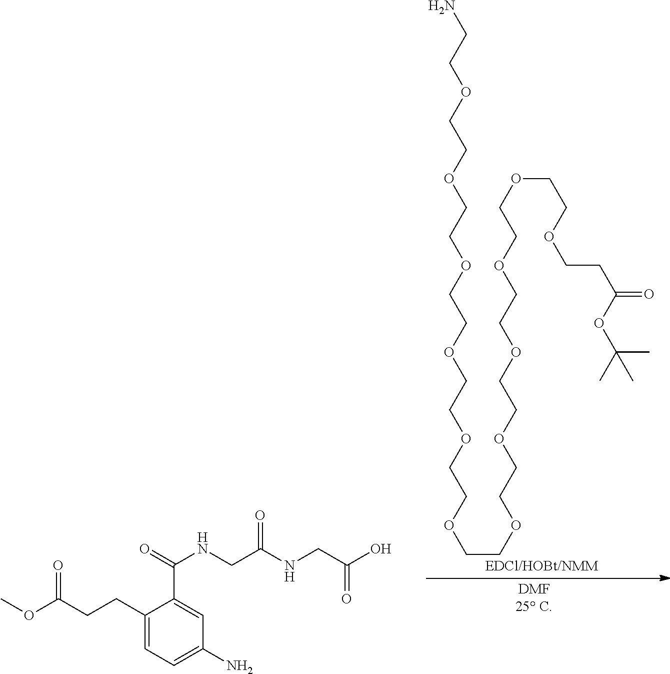 [SMILES: COC(=O)CCc1ccc(N)cc1C(=O)NCC(=O)NCC(=O)O ; CC(C)(C)OC(=O)CCOCCOCCOCCOCCOCCOCCOCCOCCOCCOCCOCCOCCN]

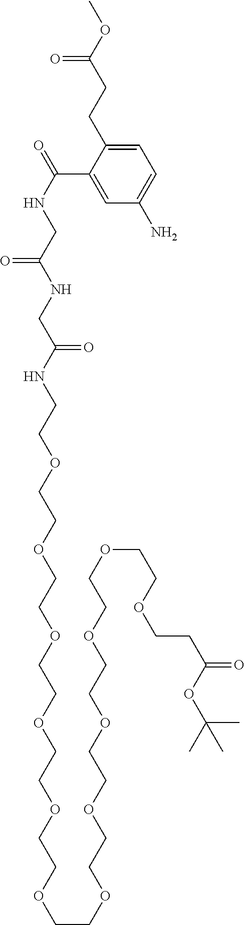 [SMILES: COC(=O)CCc1ccc(N)cc1C(=O)NCC(=O)NCC(=O)NCCOCCOCCOCCOCCOCCOCCOCCOCCOCCOCCOCCOCCC(=O)OC(C)(C)C]

A mixture of (5-amino-2-(3-methoxy-3-oxopropyl)benzoyl)glycylglycine (320.0 mg, 0.86 mmol, 1.0 eq.), tert-butyl 3-[2-[2-[2-[2-[2-[2-[2-[2-[2-[2-[2-(2-aminoethoxy)ethoxy]ethoxy]ethoxy]ethoxy]ethoxy]ethoxy]-ethoxy]ethoxy]ethoxy]ethoxy]ethoxy]propanoate (576.0 mg, 0.86 mmol, 1.0 eq.), N-(3-dimethylaminopropyl)-N′-ethylcarbodiimide hydrochloride (328.0 mg, 1.71 mmol, 2.0 eq.), 1-hydroxybenzotriazole (57.8 mg, 0.43 mmol, 0.5 eq.) and 4-methylmorpholine (259.0 mg, 2.57 mmol, 3.0 eq.) in N,N-dimethylformamide (5.0 mL) was stirred at 25° C. for 2 hours. The reaction mixture was quenched by addition water (10 mL) at 0° C., and then extracted with methylene chloride/isopropyl alcohol (v/v=3:1, 100 mL×5). The combined organic layers were concentrated under reduced pressure to give a residue. Column, C₁₈ silica gel; mobile phase, acetonitrile in water (0.1% trifluoroacetic acid/formic acid), 10% to 70% gradient in 20 min; detector, UV 254 nm. 950 mg (90%) of tert-butyl 1-(5-amino-2-(3-methoxy-3-oxopropyl)phenyl)-1,4,7-trioxo-11,14,17,20,23,26,29,32,35,38,41,44-dodecaoxa-2,5,8-triazaheptatetracontan-47-oate was obtained as yellow oil. [M+H]⁺ (ESI): 994.10.

### Intermediate Example 73

### Synthesis of (S)-3-(4-amino-2-((47-(tert-butoxycarbonyl)-52,52-dimethyl-2,5,45,50-tetraoxo-9,12,15,18,21,24,27,30,33,36,39,42,51-tridecaoxa-3,6,46-triazatripentacontyl)carbamoyl)phenyl)propanoic acid

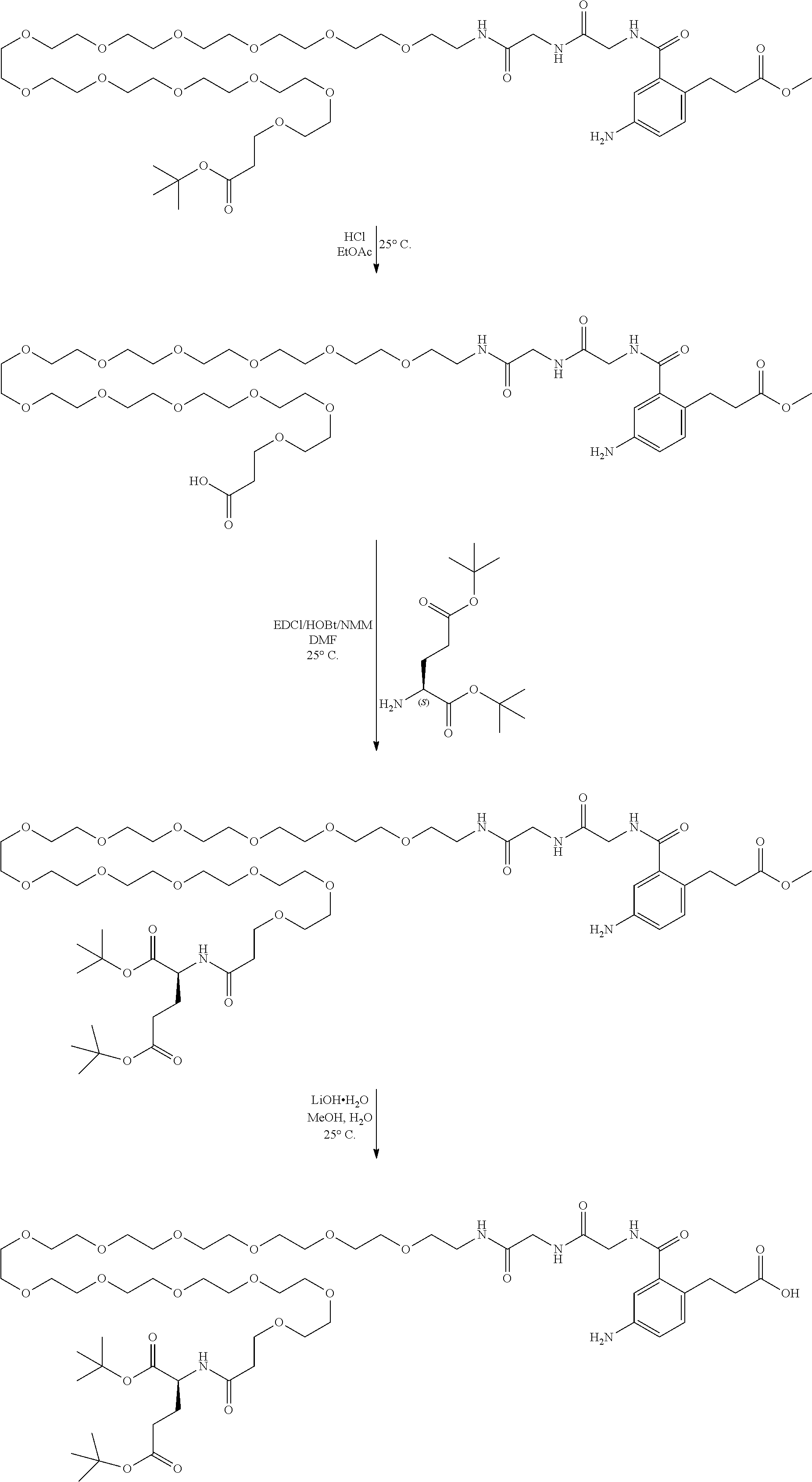

To a solution of tert-butyl 1-(5-amino-2-(3-methoxy-3-oxopropyl)phenyl)-1,4,7-trioxo-11,14,17,20,23,26,29,32,35,38,41,44-dodecaoxa-2,5,8-triazaheptatetracontan-47-oate (850 mg, 956 μmol, 1.0 eq.) in dioxane (5.0 mL) was added hydrochloride (gas)/ethyl acetate (15 mL, 4 mol/L). The mixture was stirred at 25° C. for 2 hours. The reaction mixture was concentrated under reduced pressure. 1-(5-amino-2-(3-methoxy-3-oxopropyl)phenyl)-1,4,7-trioxo-11,14,17,20,23,26,29,32,35,38,41,44-dodecaoxa-2,5,8-triazaheptatetracontan-47-oic acid (800 mg, crude) was obtained as a yellow solid. [M+H]⁺ (ESI): 936.20.

A mixture of 1-(5-amino-2-(3-methoxy-3-oxopropyl)phenyl)-1,4,7-trioxo-11,14,17,20,23,26,29,32,35,38,41,44-dodecaoxa-2,5,8-triazaheptatetracontan-47-oic acid (800 mg, 0.903 mmol, 1.0 eq.), ditert-butyl (2S)-2-aminopentanedioate (588 mg, 1.99 mmol, 2.2 eq., HCl), 1-ethyl-3(3-dimethylpropylamine) carbodiimide (346 mg, 1.81 mmol, 2.0 eq.), 1-hydroxybenzotriazole (61.0 mg, 0.451 mmol, 0.5 eq.) and 4-methylmorpholine (274 mg, 2.71 mmol, 3.0 eq.) in N,N-dimethylformamide (10.0 mL) was stirred at 25° C. for 2 hours. The reaction mixture was quenched by addition water (20 mL) at 0° C., and then extracted with methylene chloride/isopropyl alcohol (v:v=3:1, 50 mL×5). The combined organic layers were concentrated under reduced pressure to give a residue. Column, C₁₈ silica gel; mobile phase, acetonitrile in water (0.1% trifluoroacetic acid/formic acid), 10% to 70% gradient in 20 min; detector, UV 254 nm. 900 mg (89%) of di-tert-butyl (1-(2-(5-amino-2-(3-methoxy-3-oxopropyl)benzamido)acetamido)-2-oxo-6,9,12,15,18,21,24,27,30,33,36,39-dodecaoxa-3-azadotetracontan-42-oyl)-L-glutamate was obtained as yellow oil. [M+H]⁺ (ESI): 1179.05.

To a solution of di-tert-butyl (1-(2-(5-amino-2-(3-methoxy-3-oxopropyl)benzamido)acetamido)-2-oxo-6,9,12,15,18,21,24,27,30,33,36,39-dodecaoxa-3-azadotetracontan-42-oyl)-L-glutamate (800 mg, 0.68 mmol, 1.0 eq.) in methanol (8.0 mL) and water (2.5 mL) was added sodium hydroxide (136 mg, 3.39 mmol, 5.0 eq.), and then stirred at 0° C. for 2 h. The mixture was concentrated in vacuum to remove methanol, and then diluted with water (20 mL), the pH of the aqueous phase was adjusted to ˜7 with hydrochloric acid (2 mol/L), and extracted with methylene chloride/isopropyl alcohol (v/v=3:1, 30 mL×5). The combined organic layers were dried over sodium sulfate, filtered and concentrated under reduced pressure to give a residue to afford (S)-3-(4-amino-2-((47-(tert-butoxycarbonyl)-52,52-dimethyl-2,5,45,50-tetraoxo-9,12,15,18,21,24,27,30,33,36,39,42,51-tridecaoxa-3,6,46-triazatripentacontyl)carbamoyl)phenyl)propanoic acid (700 mg, crude) as red oil. [M+H]⁺ (ESI): 1165.10.

### Intermediate Example 74

### Synthesis of di-tert-butyl (1-(2-(5-(2,5-dioxo-2,5-dihydro-1H-pyrrol-1-yl)-2-(3-oxo-3-(2,3,5,6-tetrafluorophenoxy)propyl)benzamido)acetamido)-2-oxo-6,9,12,15,18,21,24,27,30,33,36,39-dodecaoxa-3-azadotetracontan-42-oyl)-L-glutamate

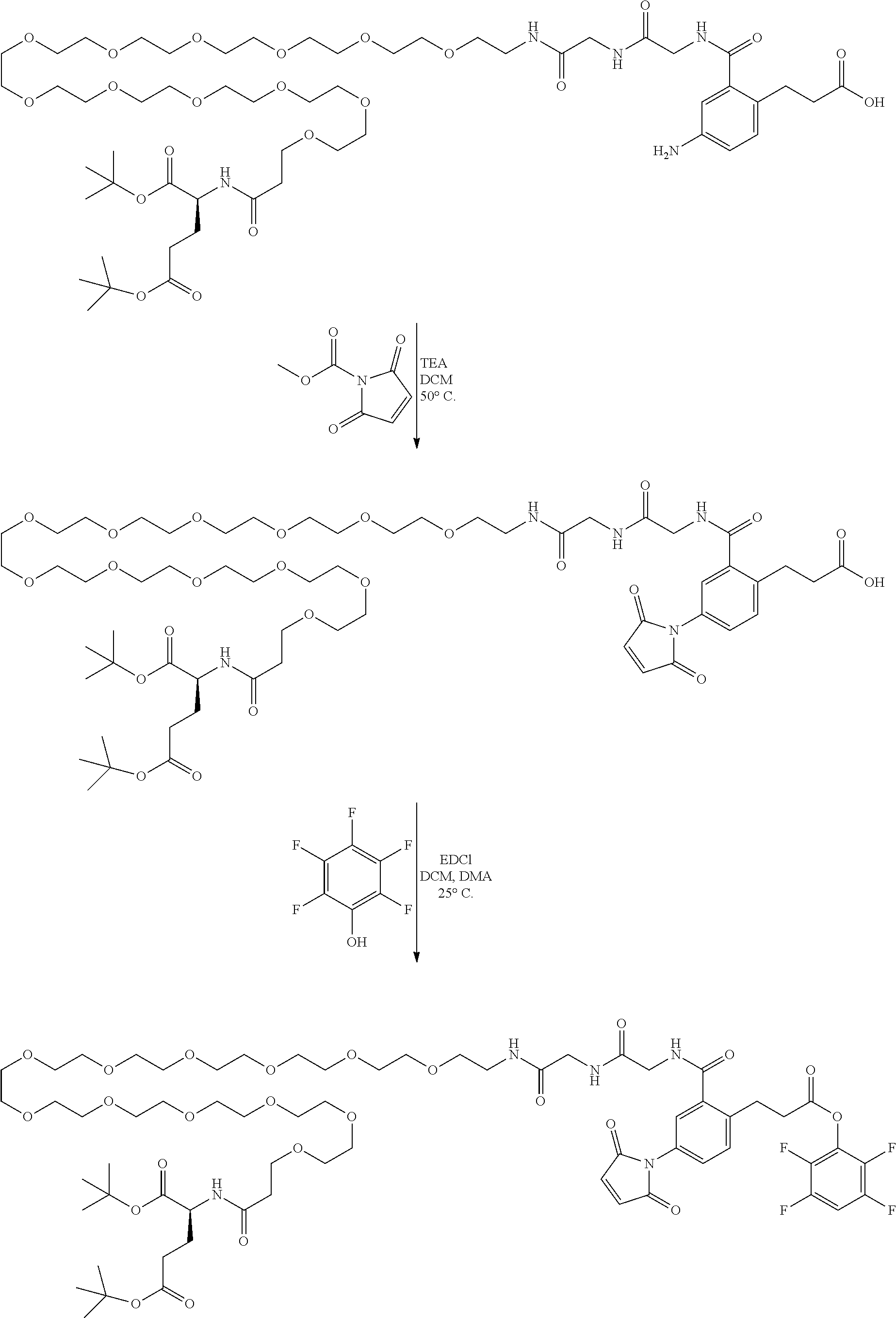 [SMILES: CC(C)(C)OC(=O)CC[C@H](NC(=O)CCOCCOCCOCCOCCOCCOCCOCCOCCOCCOCCOCCOCCNC(=O)CNC(=O)CNC(=O)c1cc(N)ccc1CCC(=O)O)C(=O)OC(C)(C)C ; COC(=O)N1C(=O)C=CC1=O ; CC(C)(C)OC(=O)CC[C@H](NC(=O)CCOCCOCCOCCOCCOCCOCCOCCOCCOCCOCCOCCOCCNC(=O)CNC(=O)CNC(=O)c1cc(N2C(=O)C=CC2=O)ccc1CCC(=O)O)C(=O)OC(C)(C)C ; Oc1c(F)c(F)c(F)c(F)c1F ; CC(C)(C)OC(=O)CC[C@H](NC(=O)CCOCCOCCOCCOCCOCCOCCOCCOCCOCCOCCOCCOCCNC(=O)CNC(=O)CNC(=O)c1cc(N2C(=O)C=CC2=O)ccc1CCC(=O)Oc1c(F)c(F)cc(F)c1F)C(=O)OC(C)(C)C]

To a solution of (S)-3-(4-amino-2-((47-(tert-butoxycarbonyl)-52,52-dimethyl-2,5,45,50-tetraoxo-9,12,15,18,21,24,27,30,33,36,39,42,51-tridecaoxa-3,6,46-triazatripentacontyl)carbamoyl)phenyl)propanoic acid (700 mg, 0.60 mmol, 1.0 eq.) and methyl 2,5-dioxopyrrole-1-carboxylate (139.8 mg, 0.92 mmol, 1.5 eq.) in dichloromethane (7.0 mL) was added trimethylamine (182.2 mg, 1.80 mmol, 3.0 eq.), and then stirred at 50° C. for 4 h. The pH of the mixture was adjusted ˜6 with trifluoroacetic acid, and diluted with addition water (10 mL) and extracted with methylene chloride:isopropyl alcohol (v/v=3:1, 30 mL×5). The combined organic layers were concentrated under reduced pressure to give a residue. Column, C₁₈ silica gel; mobile phase, acetonitrile in water (0.1% trifluoroacetic acid/formic acid), 10% to 70% gradient in 20 min; detector, UV 254 nm. 306 mg (41%) of (S)-3-(2-((47-(tert-butoxycarbonyl)-52,52-dimethyl-2,5,45,50-tetraoxo-9,12,15,18,21,24,27,30,33,36,39,42,51-tridecaoxa-3,6,46-triazatripentacontyl)carbamoyl)-4-(2,5-dioxo-2,5-dihydro-1H-pyrrol-1-yl)phenyl)propanoic acid was obtained as colorless oil. [M+H]⁺ (ESI): 1245.32.

A mixture of 2,3,5,6-tetrafluorophenol (108 mg, 0.65 mmol, 3.0 eq.), (S)-3-(2-((47-(tert-butoxycarbonyl)-52,52-dimethyl-2,5,45,50-tetraoxo-9,12,15,18,21,24,27,30,33,36,39,42,51-tridecaoxa-3,6,46-triazatripentacontyl)carbamoyl)-4-(2,5-dioxo-2,5-dihydro-1H-pyrrol-1-yl)phenyl)propanoic acid (270 mg, 216 μmol, 1.0 eq.) and 1-ethyl-3(3-dimethylpropylamine) carbodiimide (166 mg, 867 μmol, 4.0 eq.) in N,N-dimethylformamide (3.0 mL) was stirred at 25° C. for 2 h. The reaction mixture was concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 90 mg (30%) of di-tert-butyl (1-(2-(5-(2,5-dioxo-2,5-dihydro-1H-pyrrol-1-yl)-2-(3-oxo-3-(2,3,5,6-tetrafluorophenoxy)propyl)benzamido)acetamido)-2-oxo-6,9,12,15,18,21,24,27,30,33,36,39-dodecaoxa-3-azadotetracontan-42-oyl)-L-glutamate was obtained as colorless oil. [M+H]⁺ (ESI): 1394.45; ¹H NMR (DMSO-d₆, 400 MHz) δ 7.94-7.84 (m, 1H), 7.50 (d, J=8.4 Hz, 1H), 7.43-7.34 (m, 2H), 7.18 (s, 2H), 4.13 (dd, J=5.2, 9.1 Hz, 1H), 3.89 (s, 2H), 3.70 (s, 2H), 3.60-3.58 (m, 3H), 3.51-3.47 (m, 44H), 3.39 (t, J=6.0 Hz, 2H), 3.22-3.17 (m, 2H), 3.14 (s, 3H), 2.42-2.32 (m, 2H), 2.27-2.19 (m, 2H), 1.95-1.83 (m, 1H), 1.77-1.65 (m, 1H), 1.38 (s, 18H).

### Intermediate Example 75

### Synthesis of (1-(2-(5-(2,5-dioxo-2,5-dihydro-1H-pyrrol-1-yl)-2-(3-oxo-3-(2,3,5,6-tetrafluorophenoxy)propyl)benzamido)acetamido)-2-oxo-6,9,12,15,18,21,24,27,30,33,36,39-dodecaoxa-3-azadotetracontan-42-oyl)-L-glutamic acid

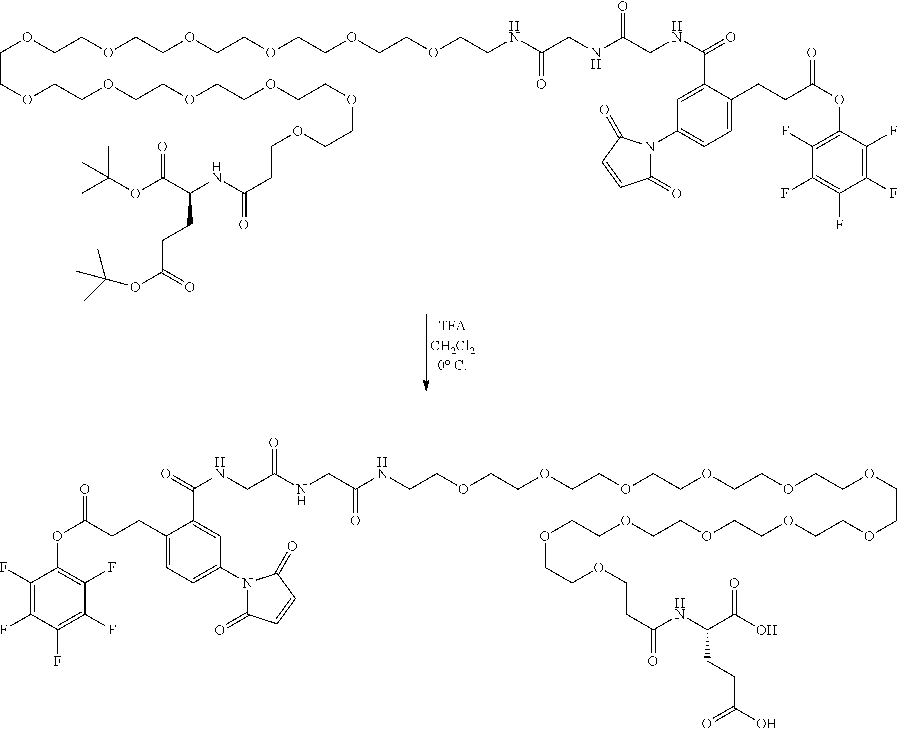 [SMILES: CC(C)(C)OC(=O)CC[C@H](NC(=O)CCOCCOCCOCCOCCOCCOCCOCCOCCOCCOCCOCCOCCNC(=O)CNC(=O)CNC(=O)c1cc(N2C(=O)C=CC2=O)ccc1CCC(=O)Oc1c(F)c(F)c(F)c(F)c1F)C(=O)OC(C)(C)C ; O=C(O)CC[C@H](NC(=O)CCOCCOCCOCCOCCOCCOCCOCCOCCOCCOCCOCCOCCNC(=O)CNC(=O)CNC(=O)c1cc(N2C(=O)C=CC2=O)ccc1CCC(=O)Oc1c(F)c(F)c(F)c(F)c1F)C(=O)O]

A solution of di-tert-butyl (1-(2-(5-(2,5-dioxo-2,5-dihydro-1H-pyrrol-1-yl)-2-(3-oxo-3-(2,3,5,6-tetrafluorophenoxy)propyl)benzamido)acetamido)-2-oxo-6,9,12,15,18,21,24,27,30,33,36,39-dodecaoxa-3-azadotetracontan-42-oyl)-L-glutamate (90 mg, 0.07 mmol, 1.0 eq.) in dichloromethane (1.0 mL) with an inert atmosphere of nitrogen, was added trifluoroacetic acid (0.25 mL) at 0° C. The resulting solution was stirred for 4 h at 0° C. and concentrated under vacuum and concentrated under reduced pressure. 90 mg (crude) of (1-(2-(5-(2,5-dioxo-2,5-dihydro-1H-pyrrol-1-yl)-2-(3-oxo-3-(2,3,5,6-tetrafluorophenoxy)propyl)benzamido)acetamido)-2-oxo-6,9,12,15,18,21,24,27,30,33,36,39-dodecaoxa-3-azadotetracontan-42-oyl)-L-glutamic acid was obtained as a yellow oil. [M+H]⁺ (ESI): 1279.95.

### Synthetic Example 1

### Preparation of Compound I-15

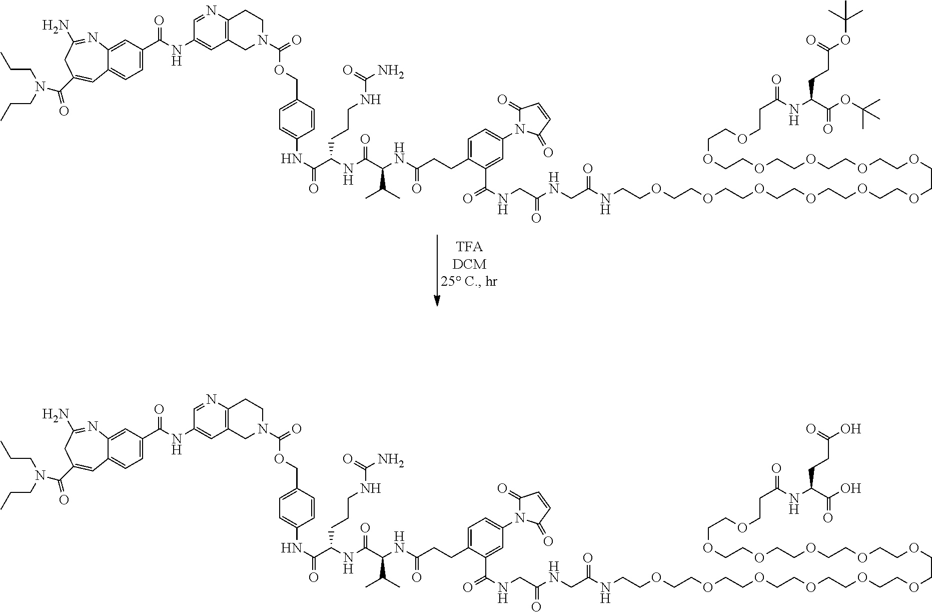 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCc5ccc(N6C(=O)C=CC6=O)cc5C(=O)NCC(=O)NCC(=O)NCCOCCOCCOCCOCCOCCOCCOCCOCCOCCOCCOCCOCCC(=O)N[C@@H](CCC(=O)OC(C)(C)C)C(=O)OC(C)(C)C)C(C)C)cc3)CC4)cc2N=C(N)C1 ; CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCc5ccc(N6C(=O)C=CC6=O)cc5C(=O)NCC(=O)NCC(=O)NCCOCCOCCOCCOCCOCCOCCOCCOCCOCCOCCOCCOCCC(=O)N[C@@H](CCC(=O)O)C(=O)O)C(C)C)cc3)CC4)cc2N=C(N)C1]

To a solution of Intermediate 7 (55.0 mg, 26.2 μmol, 1.0 eq) in DCM (1 mL) was added TFA (1.54 g, 13.5 mmol, 1.00 mL, 513 eq). The mixture was stirred at 25° C. for 2 hours. The reaction mixture was concentrated in vacuo to give a residue, the residue was purified by prep-HPLC (column: Phenomenex Luna 80×30 mm×3 μm; mobile phase: [water (TFA)-acetonitrile]; B %: 15%-45%, 8 min). The eluent was removed under freeze drying. Desired product (8.50 mg, 4.04 μmol, 15.3% yield, 99.4% purity, TFA) was obtained as a white solid.

¹H NMR (D₂O, 400 MHz) δ 9.05 (s, 1H), 8.31 (s, 1H), 7.91-7.76 (m, 2H), 7.63 (d, J=8.6 Hz, 1H), 7.46-7.16 (m, 7H), 7.09 (s, 1H), 6.93 (s, 2H), 5.14 (s, 2H), 4.43-4.34 (m, 1H), 4.32-4.24 (m, 1H), 4.17-4.02 (m, 2H), 3.97-3.79 (m, 5H), 3.72 (t, J=6.0 Hz, 2H), 3.68-3.55 (m, 46H), 3.53 (t, J=6.0 Hz, 2H), 3.48-3.30 (m, 8H), 3.16-3.11 (m, 2H), 3.08-2.97 (m, 4H), 2.66-2.61 (m, 2H), 2.58-2.48 (m, 2H), 2.42 (t, J=7.2 Hz, 2H), 2.21-2.07 (m, 1H), 1.98-1.85 (m, 2H), 1.85-1.75 (m, 1H), 1.73-1.56 (m, 5H), 1.54-1.34 (m, 2H), 0.89 (t, J=7.2 Hz, 3H), 0.79 (t, J=6.0 Hz, 9H)

HPLC: 99.45% (220 nm), 100.00% (254 nm)

MS (ESI): mass calcd. For C₉₅H₁₃₄N₁₆O₃₀ 1978.95, m/z found 1979.9641 [M+H]⁺

### Synthetic Example 2

### Preparation of Compound I-13

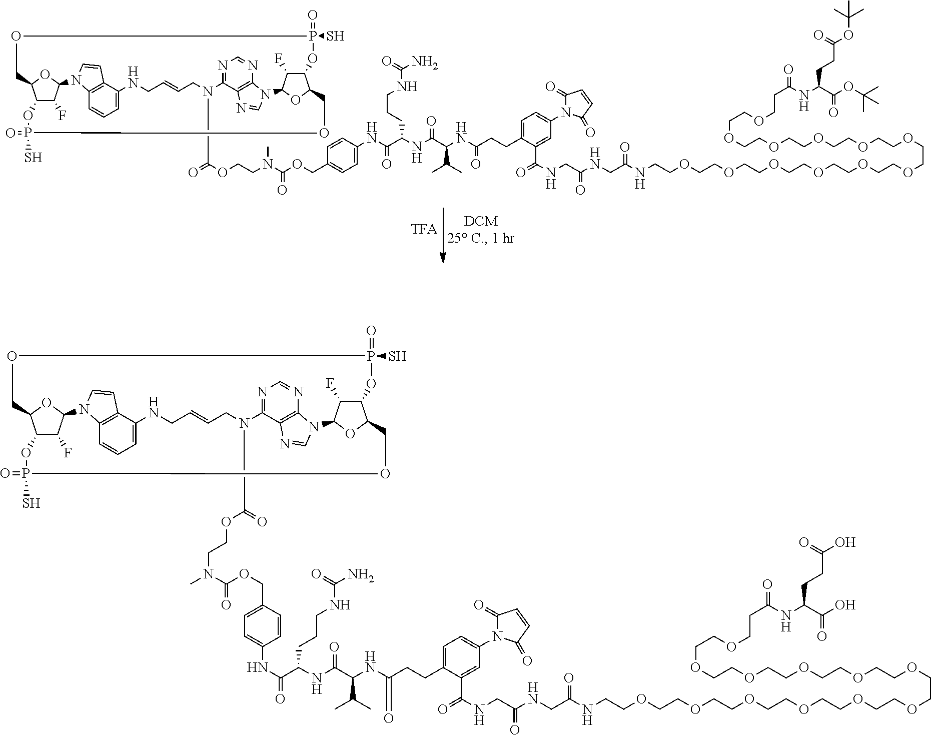

To a solution of Intermediate 8b (14 mg, 5.65 μmol, 1.0 eq) in DCM (0.5 mL) was added TFA (770 mg, 6.75 mmol, 0.5 mL, 1196 eq). The mixture was stirred at 25° C. for 1 hr. The mixture was concentrated in vacuo. The residue was purified by prep-HPLC (column: C₁₈₋₄ 150·30 mm·5 μm; mobile phase: [A: 0.1 M water (TFA) B: acetonitrile]; B %: 15%-45%, 20 min) to afford the desired product (2 mg, 8.45e-1 μmol, 14.96% yield) as white solid.

¹H NMR (400 MHz, DMSO-d₆) δ9.71 (s, 1H), 8.99 (s, 1H), 8.74 (s, 1H), 8.40-8.32 (m, 1H), 8.10-7.92 (m, 1H), 7.84-7.75 (m, 2H), 7.65-7.53 (m, 4H), 7.43-7.29 (m, 3H), 7.21 (d, J=8.4 Hz, 2H), 7.11 (s, 2H), 6.47-6.37 (m, 2H), 5.75-5.61 (m, 2H), 4.90 (s, 2H), 4.87-4.70 (m, 3H), 4.69-4.59 (m, 1H), 4.49-4.40 (m, 1H), 4.39-4.11 (m, 8H), 3.94 (d, J=6.0 Hz, 2H), 3.87-3.80 (m, 1H), 3.78-3.69 (m, 3H), 3.65-3.62 (m, 2H), 3.56-3.49 (m, 44H), 3.47-3.43 (m, 2H), 3.27-3.22 (m, 2H), 2.68-2.66 (m, 2H), 2.60 (s, 3H), 2.40-2.36 (m, 2H), 2.34-2.31 (m, 3H), 2.30-2.24 (m, 3H), 2.04-1.97 (m, 3H), 1.87-1.74 (m, 3H), 1.70-1.60 (m, 1H), 1.49-1.39 (m, 2H), 1.27 (s, 3H), 0.85 (t, J=7.2 Hz, 6H).

MS (ESI): mass calcd. For C₉₈H₁₃₆F₂N₂₀O₃₈P₂S₂ 2364.82, m/z found 1184.1 [M/2+H]⁺

HPLC: 96.65% (220 nm), 95.84% (254 nm)

### Synthetic Example 3

### Preparation of Compound I-49

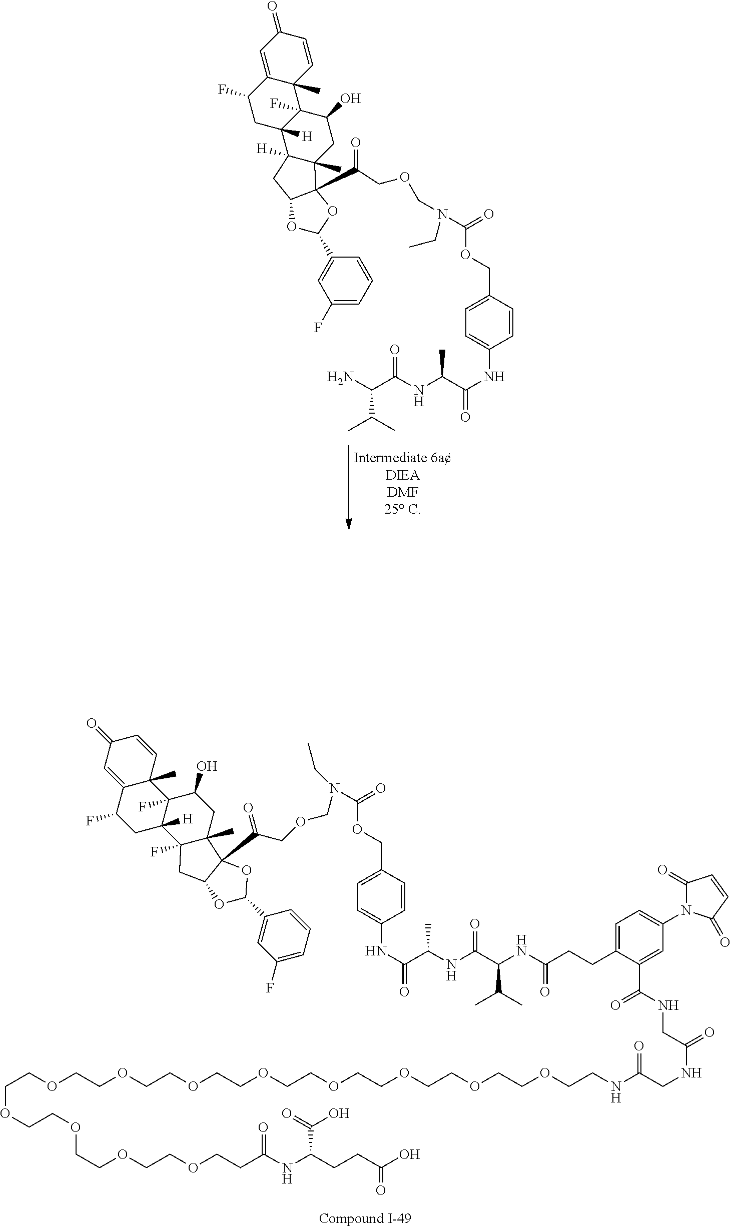

To a solution of [4-[[(2S)-2-[[(2S)-2-amino-3-methyl-butanoyl]amino]propanoyl]amino]phenyl]methyl N-[[2-[(1S,2S,4R,6R,8S,9S,11S,12R,13S,19S)-12,19-difluoro-6-(3-fluoro phenyl)-11-hydroxy-9,13-dimethyl-16-oxo-5,7-dioxapentacyclo[10.8.0.02,9.04,8.013,18]icosa-14,17-dien-8-yl]-2-oxo-ethoxy]methyl]-N-ethyl-carbamate (94.3 mg, 51.4 μmol, 55% purity, 1.0 eq, TFA) in DMF (0.7 mL) was added DIEA (19.9 mg, 154 μmol, 26.8 μL, 3.0 eq) and (2S)-2-[3-[2-[2-[2-[2-[2-[2-[2-[2-[2-[2-[2-[2-[[2-[2-[[5-(2,5-dioxopyrrol-1-yl)-2-[3-oxo-3-(2,3,5,6-tetrafluorophenoxy)propyl]benzoyl]amino]ethylamino]acetyl]amino]ethoxy]ethoxy]ethoxy]ethoxy]ethoxy]ethoxy]ethoxy]ethoxy]ethoxy]ethoxy]ethoxy]ethoxy]propanoylamino]pentanedioic acid (Intermediate 6a′, 78.0 mg, 56.5 μmol, 1.1 eq, TFA) at 0° C. The mixture was stirred at 25° C. for 1 hr. The reaction mixture was quenched with HCOOH until pH=˜7. The reaction mixture was purified by prep-HPLC (column: Phenomenex Luna C₁₈ 200×40 mm×10 μm; mobile phase: [water (FA)-ACN]; B %: 35%-65%, 8 min). (2S)-2-[3-[2-[2-[2-[2-[2-[2-[2-[2-[2-[2-[2-[2-[[2-[[2-[[2-[3-[[(1S)-1-[[(1S)-2-[4-[[[2-[(1S,2S,4R,6R,8S,9S,11S,12R,13S,19S)-12,19-Difluoro-6-(3-fluorophenyl)-11-hydroxy-9,13-dimethyl-16-oxo-5,7-dioxapentacyclo[10.8.0.02,9.04,8.013,18]icosa-14,17-dien-8-yl]-2-oxo-ethoxy]methyl-ethyl-carbamoyl]oxymethyl]anilino]-1-methyl-2-oxo-ethyl]carbamoyl]-2-methyl-propyl]amino]-3-oxo-propyl]-5-(2,5-dioxopyrrol-1-yl)benzoyl]amino]acetyl]amino]-acetyl]amino]ethoxy]ethoxy]ethoxy]ethoxy]ethoxy]ethoxy]ethoxy]ethoxy]-ethoxy]ethoxy]ethoxy]ethoxy]propanoylamino]pentanedioic acid, Compound I-49 (21.9 mg, 10.85 μmol, 21.11% yield, 99.51% purity) was obtained as a white solid: ¹H NMR (400 MHz, DMSO-d₆) δ 9.97-9.80 (m, 1H), 8.70-8.62 (m, 1H), 8.27-8.16 (m, 2H), 7.92-7.85 (m, 2H), 7.64-7.16 (m, 12H), 7.14-7.08 (m, 2H), 6.29 (dd, J=1.6, 10.4 Hz, 1H), 6.12 (s, 1H), 5.72-5.47 (m, 2H), 5.05-4.89 (m, 3H), 4.86-4.57 (m, 3H), 4.38-4.24 (m, 2H), 4.19-4.17 (m, 2H), 4.12 (t, J=7.6 Hz, 1H), 3.92-3.86 (m, 2H), 3.74-3.69 (m, 2H), 3.60-3.54 (m, 3H), 3.49-3.44 (m, 44H), 3.38 (t, J=5.6 Hz, 3H), 3.31-3.27 (m, 1H), 3.22-3.19 (m, 2H), 3.01-2.91 (m, 2H), 2.38-2.26 (m, 4H), 2.20-2.09 (m, 1H), 1.99-1.87 (m, 3H), 1.78-1.64 (m, 4H), 1.47 (s, 3H), 1.27 (d, J=7.2 Hz, 3H), 1.06 (t, J=6.8 Hz, 3H), 0.87-0.74 (m, 9H); HPLC: 99.51% (220 nm), 98.86% (254 nm); LC/MS [M+H] 2008.9 (calculated); LC/MS [M+H] 2008.9 (observed).

Compounds in the table below were prepared in a manner similar to the methods and protocols of and preceding Synthetic Example 3 (see also, e.g., Intermediate Examples 25-32) using suitable compounds as starting materials.

| Cmpd. | Structure | MS/¹H NMR |
|---|---|---|
| I-35 | 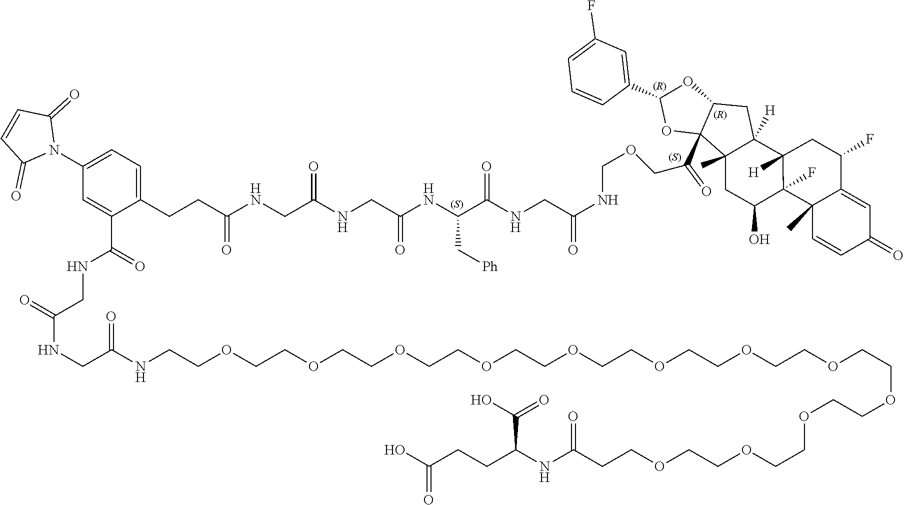 | MS m/z [M − H]⁻ (ESI): 1977.90; ¹H NMR (300 MHz, Methanol-d₄) δ 0.79 (d, J = 7.1 Hz, 3H), 0.89 (s, 3H), 1.08 (t, J = 7.1 Hz, 2H), 1.13-1.37 (m, 7H), 1.47 (s, 4H), 1.62-1.76 (m, 3H), 1.73-1.89 (m, 1H), 2.11 (d, J = 12.7 Hz, 3H), 2.24 (t, J = 7.2 Hz, 3H), 2.31-2.45 (m, 2H), 2.49-2.62 (m, 2H), 2.78-3.16 (m, 4H), 3.28 (d, J = 5.4 Hz, 2H), 3.40-3.48 (m, 2H), 3.48-3.58 (m, 36H), 3.56-3.74 (m, 6H), 3.79 (d, J = 11.7 Hz, 3H), 3.98 (s, 2H), 4.15-4.30 (m, 2H), 4.30-4.45 (m, 2H), 4.55-4.77 (m, 4H), 4.95 (d, J = 4.3 Hz, 1H), 5.39 (s, 1H), 5.51 (s, 1H), 6.13-6.24 (m, 2H), 6.88 (s, 1H), 7.01 (t, J = 8.9 Hz, 1H), 7.07-7.25 (m, 7H), 7.23-7.32 (m, 1H), 7.32-7.49 (m, 2H) |
| I-36 | 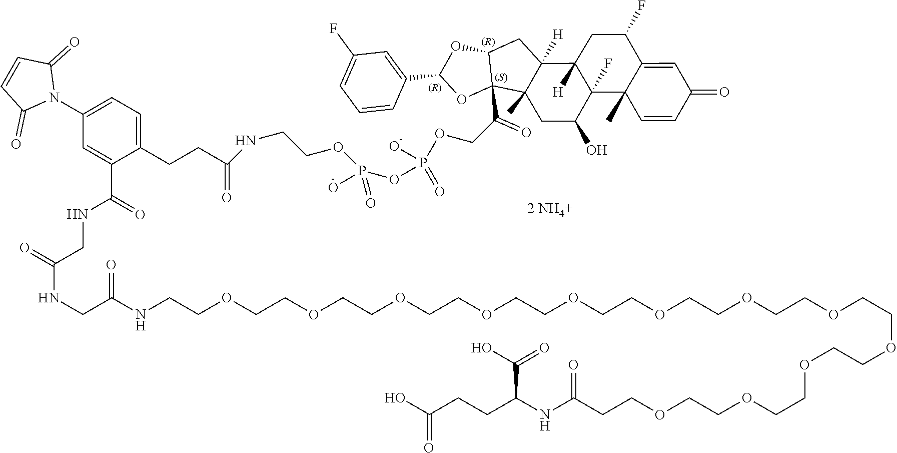 | MS m/z [M − H]⁻ (ESI): 1834.50; ¹H NMR (300 MHz, Methanol-d₄) δ: 0.75-1.12 (m, 3H), 1.20-1.43 (m, 1H), 1.51-1.68 (m, 4H), 1.70-1.84 (m, 2H), 1.85-2.06 (m, 2H), 2.05-2.42 (m, 6H), 2.45-2.61 (m, 4H), 2.69 (d, J = 31.9 Hz, 1H), 3.08 (s, 2H), 3.41 (t, J = 5.7 Hz, 2H), 3.53-3.59 (m, 2H), 3.59-3.66 (m, 43H), 3.74 (t, J = 6.2 Hz, 2H), 3.90 (d, J = 21.1 Hz, 4H), 4.11 (d, J = 4.1 Hz, 2H), 4.23 (dd, J = 7.9 Hz, 4.2 Hz, 1H), 4.32 (d, J = 9.7 Hz, 1H), 4.92-5.16 (m, 4H), 5.34-5.54 (m, 1H), 5.60 (s, 1H), 5.90-6.03 (m, 1H), 6.26-6.63 (m, 3H), 6.82-7.46 (m, 6H), 7.56-7.66 (m, 1H), 7.73-7.88 (m, 1H) |
| I-37 | 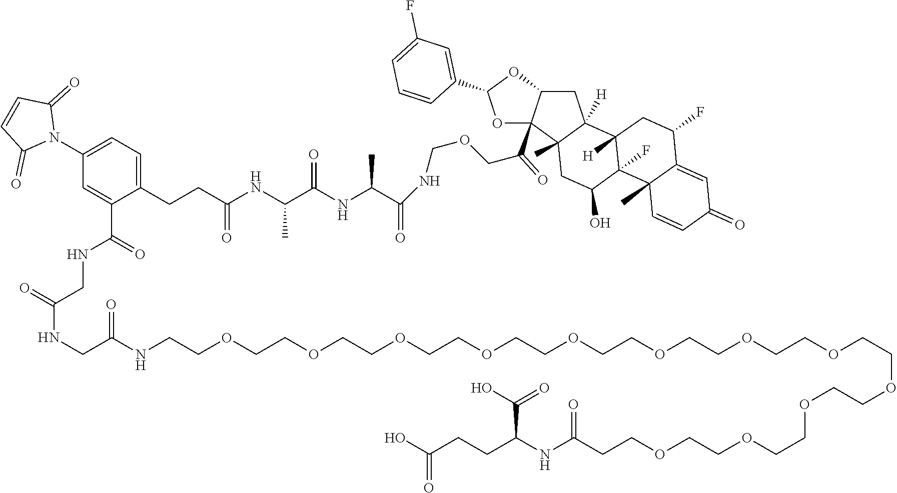 | MS m/z [M − H]⁻ (ESI): 1801.60; ¹H NMR (300 MHz, Methanol-d₄) δ: 0.98 (s, 3H), 1.27-1.34 (m, 6H), 1.57 (s, 4H), 1.80-1.91 (m, 4H), 2.10-2.20 (m, 2H), 2.37-2.39 (m, 4H), 2.63-2.70 (m, 5H), 3.13-3.20 (m, 2H), 3.37-3.40 (m, 3H), 3.52-3.59 (m, 6H), 3.61-3.62 (m, 37H), 3.72-3.74 (m, 2H), 3.92 (s, 3H), 4.09 (s, 2H), 4.17-4.23 (m, 3H), 4.26-4.43 (m, 2H), 4.66-4.75 (m, 4H), 5.03-5.04 (m, 1H), 5.50-5.58 (m, 2H), 6.31-6.34 (m, 2H), 6.97 (s, 2H), 7.10-7.20 (m, 2H), 7.32-7.47 (m, 6H). |

### Synthetic Example 4

### Synthesis of Compound I-44

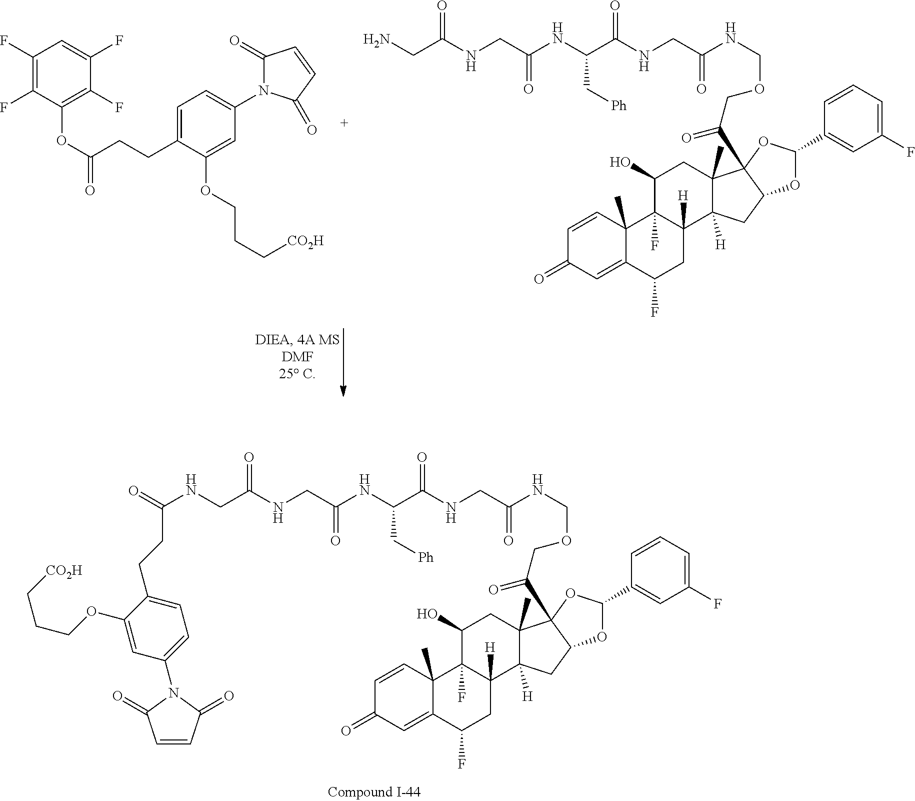

A solution of 4-(5-(2,5-dioxo-2,5-dihydro-1H-pyrrol-1-yl)-2-(3-oxo-3-(2,3,5,6-tetrafluorophenoxy)propyl)phenoxy)butanoic acid (120.0 mg, 0.20 mmol, 1.0 eq.) in N,N-dimethylformamide (2.0 mL) was treated with N,N-diisopropylethylamine (52.3 mg, 0.40 mmol, 2.0 eq.) at room temperature. To the above mixture was added (S)-2-(2-(2-aminoacetamido)acetamido)-N-(2-(((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)amino)-2-oxoethyl)-3-phenylpropanamide (192.6 mg, 0.22 mmol, 1.1 eq.) at 25° C. The resulting mixture was stirred for additional 5 h at 25° C. The crude product was purified by Prep-HPLC with the following conditions (acetonitrile/water with 0.05% formic acid). 64 mg (26%) of Compound I-44 was obtained as a white solid. [M−H]⁻ (ESI): 1193.40. ¹H NMR (300 MHz, Methanol-d₄) δ 0.98 (s, 3H), 1.57 (s, 4H), 1.64-1.90 (m, 3H), 1.99-2.23 (m, 3H), 2.24-2.4 (m, 2H), 2.42-2.82 (m, 5H), 2.86-3.08 (m, 3H), 3.20 (dd, J=13.9 Hz, 5.8 Hz, 1H), 3.68-3.95 (m, 6H), 4.02 (t, J=6.1 Hz, 2H), 4.24-4.38 (m, 1H), 4.39-4.52 (m, 2H), 4.62 (d, J=28.1 Hz, 1H), 4.70-4.81 (m, 2H), 5.04 (d, J=4.4 Hz, 1H), 5.37-5.71 (m, 2H), 6.16-6.35 (m, 2H), 6.81 (dd, J=8.0 Hz, 1.9 Hz, 1H), 6.88-6.98 (m, 3H), 7.03-7.17 (m, 1H), 7.14-7.44 (m, 10H).

The compound in the table below was prepared in a manner similar to the methods and protocols of and preceding Synthetic Example 4 using suitable compounds as starting materials.

| Cmpd. | Structure | MS/¹H NMR |
|---|---|---|
| I-43 | 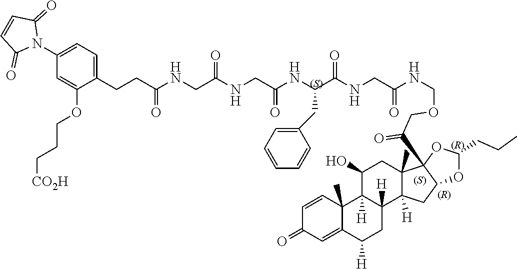 | MS m/z [M − H]⁻ (ESI): 1105.40; ¹H NMR (300 MHz, Methanol-d₄) δ: 0.88-0.98 (m, 6H), 0.99-1.02 (m, 1H), 1.03-1.19 (m, 1H), 1.27 (s, 1H), 1.39-1.44 (m, 2H), 1.47 (s, 3H), 1.51-1.64 (m, 4H), 1.66-1.73 (m, 1H), 1.81-1.89 (m, 3H), 2.02-2.33 (m, 4H), 2.31- 2.39 (m, 1H), 2.46-2.53 (m, 2H), 2.58-2.69 (m, 3H), 2.91-3.09 (m, 3H), 3.11-3.19 (m, 1H), 3.21-3.29 (m, 1H), 3.71-2.91 (m, 6H), 3.92-4.11 (m, 2H), 4.39-4.41 (m, 1H), 4.48- 4.58 (m, 2H), 4.61-4.66 (m, 1H), 4.69-4.79 (m, 2H), 5.99 (s, 1H), 6.12-6.28 (m, 1H), 6.76-6.88 (m, 1H), 6.89-6.99 (m, 3H), 7.11- 7.31 (m, 6H), 7.32-7.44 (m, 1H) |

### Synthetic Example 5

### Synthesis of Compound I-46

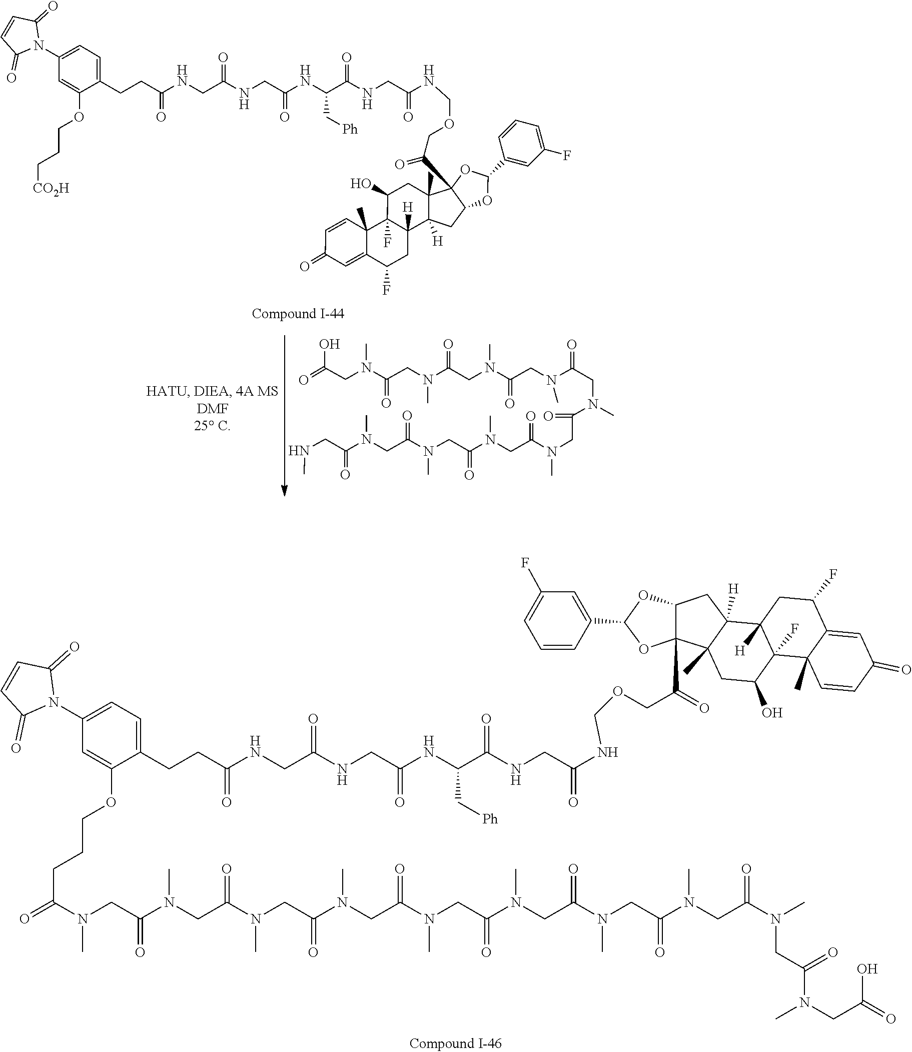

Compound I-46 was synthesized using the conditions and materials according to the reaction scheme shown above.

[M−H]− (ESI): 1904.75; 1H NMR (300 MHz, Methanol-d4) δ 0.98 (s, 4H), 1.29 (s, 1H), 1.56 (s, 4H), 1.79 (d, J=13.8 Hz, 3H), 2.01-2.24 (m, 4H), 2.33 (s, 2H), 2.52-2.78 (m, 5H), 2.81-3.24 (m, 33H), 3.61-3.90 (m, 6H), 3.90-4.23 (m, 12H), 4.24-4.53 (m, 11H), 4.66-4.81 (m, 3H), 5.03 (d, J=4.4 Hz, 1H), 5.60 (s, 2H), 6.28 (d, J=11.7 Hz, 2H), 6.74-7.16 (m, 5H), 7.16-7.42 (m, 10H).

The compound in the table below was prepared in a manner similar to the methods and protocols of and preceding Synthetic Example 5 using suitable compounds as starting materials.

| Cmpd. | Structure | MS/¹H NMR |
|---|---|---|
| I-45 | 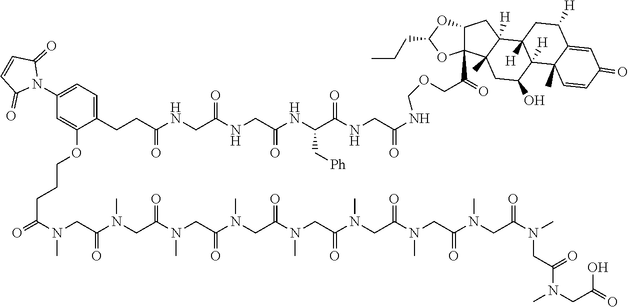 | MS m/z [M − H]− (ESI): 1816.80; 1H NMR (400 MHz, Methanol-d4) δ: 0.79-0.88 (m, 6H), 0.89-0.92 (m, 1H), 0.94-1.04 (m, 1H), 1.13-1.24 (m, 5H), 1.26-1.34 (m, 3H), 1.37 (s, 3H), 1.42- 1.51 (m, 4H), 1.58-1.62 (m, 1H), 1.69-1.71 (m, 2H), 1.91-2.19 (m, 5H), 2.21-2.29 (m, 1H), 2.46-2.61 (m, 4H), 2.79-2.88 (m, 15H), 2.89-2.99 (m, 13H), 3.11-3.18 (m, 1H), 3.66-3.81 (m, 6H), 3.91-4.01 (m, 4H), 4.02-4.18 (m, 7H), 4.21-4.24 (m, 3H), 4.26-4.32 (m, 5H), 4.39-4.48 (m, 2H), 4.49-4.58 (m, 3H), 4.61-4.68 (m, 3H), 4.71-4.76 (m, 2H), 5.89 (s, 1H), 5.99-6.22 (m, 1H), 6.61-6.78 (m, 1H), 6.79-6.98 (m, 3H), 7.01-7.18 (m, 2H), 7.19-7.24 (m, 4H), 7.26-7.38 (m, 1H). |

### Synthetic Example 6

### Synthesis of Compound I-47

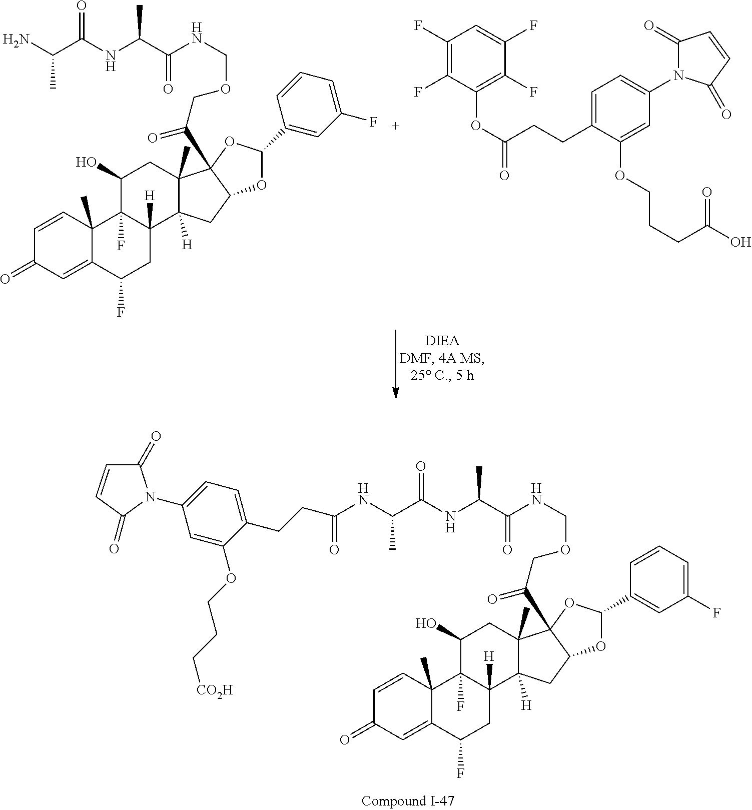

To a stirred solution of 4-(5-(2,5-dioxo-2,5-dihydro-1H-pyrrol-1-yl)-2-(3-oxo-3-(2,3,5,6-tetrafluorophenoxy)propyl)phenoxy)butanoic acid (200.0 mg, 0.40 mmol, 1.0 eq.) and (S)-2-amino-N—((S)-1-(((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)amino)-1-oxopropan-2-yl)propanamide (417.7 mg, 0.60 mmol, 1.5 eq.) in N,N-dimethylformamide (5.0 mL), was added N,N-diisopropylethylamine (156.5 mg, 1.20 mmol, 3.0 eq.) dropwise in at 25° C. The resulting mixture was stirred for additional 5 h at 25° C. The crude product was purified by Flash with the following conditions (IntelFlash-1): Column, C₁₈ silica gel; mobile phase, water (with 5 mmol/L formic acid) and acetonitrile (10.0% acetonitrile up to 100.0% in 20 min); Detector, UV 254 nm. 150 mg (36%) of Compound I-47 was obtained as white solid: MS m/z [M+H]⁺ (ESI): 1019.38.

Compounds in the table below were prepared in a manner similar to the methods and protocols of and preceding Synthetic Example 6 (see also, e.g., Intermediate Examples 33-37) using suitable compounds as starting materials.

| Cmpd. | Structure | MS/¹H NMR |
|---|---|---|
| I-56 | 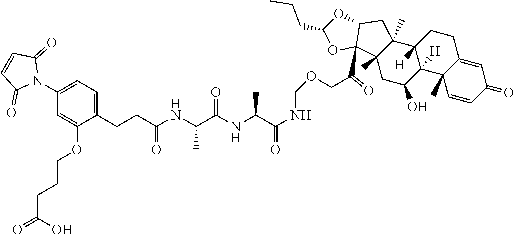 | — |
| I-41 | 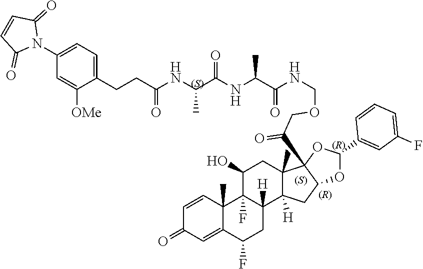 | MS m/z [M − H]− (ESI): 945.30; 1H NMR (400 MHz, Methanol-d4) δ: 0.99 (s, 3H), 1.27-1.29 (m, 3H), 1.35-1.36 (m, 3H), 1.56 (s, 4H), 1.78-1.81 (m, 3H), 2.15-2.25 (m, 1H), 2.30-2.34 (m, 2H), 2.50-2.54 (m, 2H), 2.60-2.70 (m, 1H), 2.91-2.99 (m, 2H), 3.81 (s, 3H), 4.21- 4.28 (m, 3H), 4.39-4.44 (m, 1H), 4.68- 4.77 (m, 3H), 5.04-5.05 (m, 1H), 5.50- 5.58 (m, 2H), 6.30-6.33 (m, 2H), 6.79- 6.81 (m, 1H), 6.89-6.94 (m, 3H), 7.11- 7.18 (m, 1H), 7.20-7.29 (m, 2H), 7.31- 7.39 (m, 3H). |

### Synthetic Example 7

### Synthesis of Compound I-48

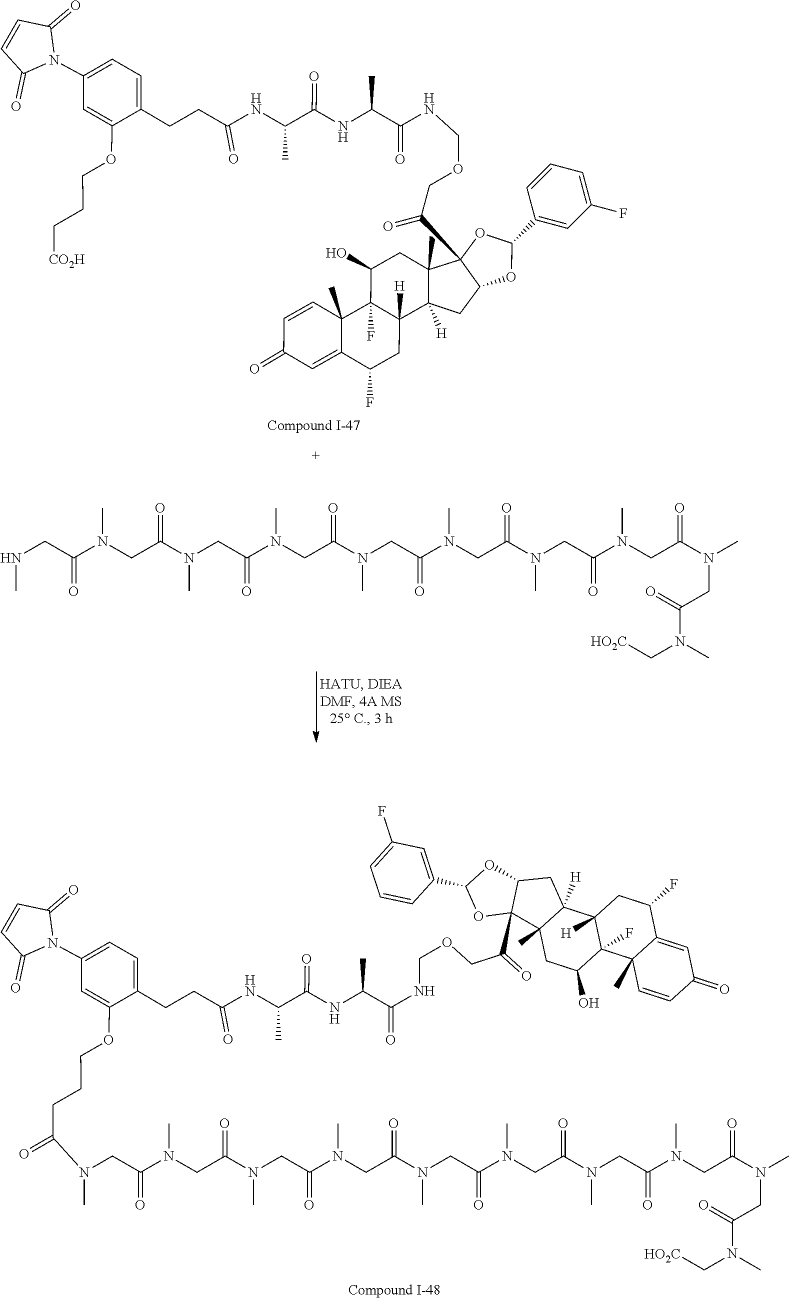

To a stirred solution of Compound I-47 (100.0 mg, 0.098 mmol, 1.0 eq.) and HATU (2-(3H-[1,2,3]triazolo[4,5-b]pyridin-3-yl)-1,1,3,3-tetramethylisouronium) (55.97 mg, 0.147 mmol, 1.5 eq.) in N,N-dimethylformamide (2.0 mL) were added N,N-diisopropylethylamine (38.05 mg, 0.294 mmol, 3.0 eq.) and (5,8,11,14,17,20,23,26,29-nonamethyl-4,7,10,13,16,19,22,25,28-nonaoxo-2,5,8,11,14,17,20,23,26,29-decaazahentriacontan-31-oic acid) (143.04 mg, 0.196 mmol, 2.0 eq.) dropwise at 25° C. under air atmosphere. The resulting mixture was stirred for additional 3 h at 25° C. The crude product was purified by Flash with the following conditions (IntelFlash-1): Column, C₁₈ silica gel; mobile phase, water and acetonitrile (10.0% acetonitrile up to 100.0% in 20 min); Detector, UV 254 nm. The resulting mixture was lyophilized in a cool and dry place. 20 mg (10%) of Compound I-48 was obtained as white solid: MS m/z [M+H]⁺

(ESI): 1728.05; ¹H NMR (400 MHz, Methanol-d₄) δ: 0.86-0.90 (m, 1H), 1.02 (s, 3H), 1.22-1.40 (m, 6H), 1.56 (s, 3H), 1.56-1.70 (m, 1H), 1.75-1.9 (m, 3H), 2.05-2.40 (m, 3H), 2.25-2.45 (m, 2H), 2.50-2.61 (m, 2H), 2.62-2.75 (m, 2H), 2.90-3.15 (m, 33H), 3.95-4.55 (m, 24H), 4.61-4.82 (m, 3H), 5.05-5.11 (m, 1H), 5.40-5.70 (m, 2H), 6.25-6.40 (m, 2H), 6.70-6.82 (m, 1H), 6.85-7.00 (m, 3H), 7.05-7.28 (m, 3H), 7.31-7.5 (m, 3H).

Compounds in the table below were prepared in a manner similar to the methods and protocols of and preceding Synthetic Example 7 using suitable compounds as starting materials.

| Cmpd. | Structure | MS/¹H NMR |
|---|---|---|
| I-54 | 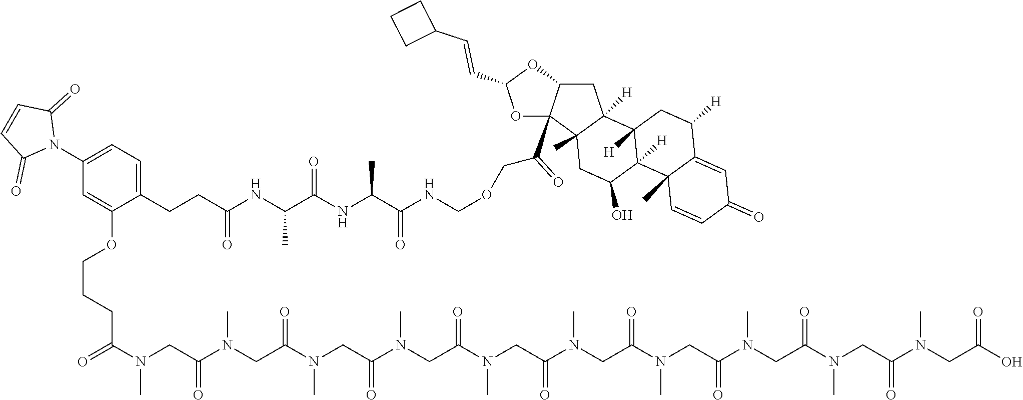 | MS m/z [M − H]⁻ (ESI): 1678.1; ¹H NMR (400 MHz, DMSO-d₆) δ 7.31 (d, J = 10.0 Hz, 1H), 7.22- 7.13 (m, 1H), 7.05 (s, 2H), 6.85 (d, J = 10.4 Hz, 1H), 6.78-6.70 (m, 1H), 6.21-6.11 (m, 1H), 6.05- 5.96 (m, 1H), 5.92 (s, 1H), 5.32- 5.22 (m, 1H), 4.91 (d, J = 6.8 Hz, 1H), 4.71 (d, J = 3.6 Hz, 1H), 4.65-4.40 (m, 3H), 4.38-3.96 (m, 24H), 3.01-2.66 (m, 36H), 2.45- 2.26 (m, 4H), 2.09-1.90 (m, 6H), 1.82-1.64 (m, 6H), 1.61-1.47 (m, 3H), 1.35 (s, 3H), 1.27-1.11 (m, 7H), 1.05-0.88 (m, 2H), 0.79 (s, 3H). |
| I-58 | 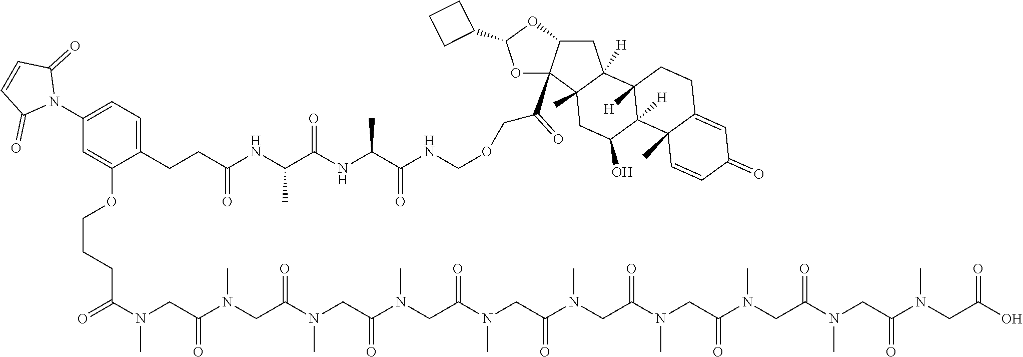 | MS m/z [M + H]⁺ (ESI): 1672.0; ¹H NMR (400 MHz, DMSO-d₆) δ 8.76-8.59 (m, 1H), 7.33-7.24 (m, 1H), 7.22-7.15 (m, 1H), 7.11 (s, 2H), 6.88 (br d, J = 8.8 Hz, 1H), 6.76 (br dd, J = 1.6, 7.6 Hz, 1H), 6.22 (dd, J = 1.6, 10.0 Hz, 1H), 6.01 (s, 1H), 4.74 (br s, 1H), 4.69- 4.42 (m, 4H), 4.38-4.14 (m, 13H), 4.13-3.86 (m, 12H), 3.06-2.67 (m, 32H), 2.61 (dt, J = 5.2, 13.2 Hz, 1H), 2.47-2.37 (m, 3H), 2.36-2.21 (m, 2H), 2.07-1.64 (m, 13H), 1.55 (br d, J = 8.4 Hz, 2H), 1.46 (s, 3H), 1.36-1.25 (m, 1H), 1.25-1.19 (m, 3H), 1.19-1.12 (m, 3H), 0.81 (s, 3H). |
| I-59 | 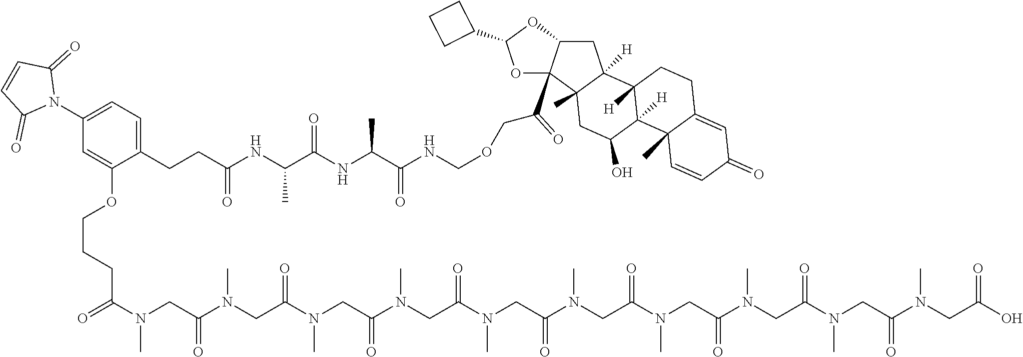 | MS m/z [M + Na]⁺ (ESI): 1675.9; ¹H NMR (400 MHz, DMSO-d₆) δ 8.75-8.57 (m, 1H), 7.30 (br d, J = 10.0 Hz, 1H), 7.24-7.14 (m, 1H), 7.08 (s, 2H), 6.87 (br d, J = 9.2 Hz, 1H), 6.75 (br dd, J = 1.6, 7.6 Hz, 1H), 6.20-6.10 (m, 1H), 5.91 (s, 1H), 4.72 (br d, J = 3.6 Hz, 1H), 4.67-4.39 (m, 4H), 4.37-4.14 (m, 13H), 4.12-3.82 (m, 12H), 3.06- 2.62 (m, 33H), 2.47-2.37 (m, 3H), 2.34-2.19 (m, 2H), 2.13-1.66 (m, 13H), 1.65-1.43 (m, 3H), 1.35 (s, 3H), 1.25-1.18 (m, 3H), 1.18-1.10 (m, 3H), 1.01-0.82 (m, 2H), 0.79 (s, 3H). |
| I-60 | 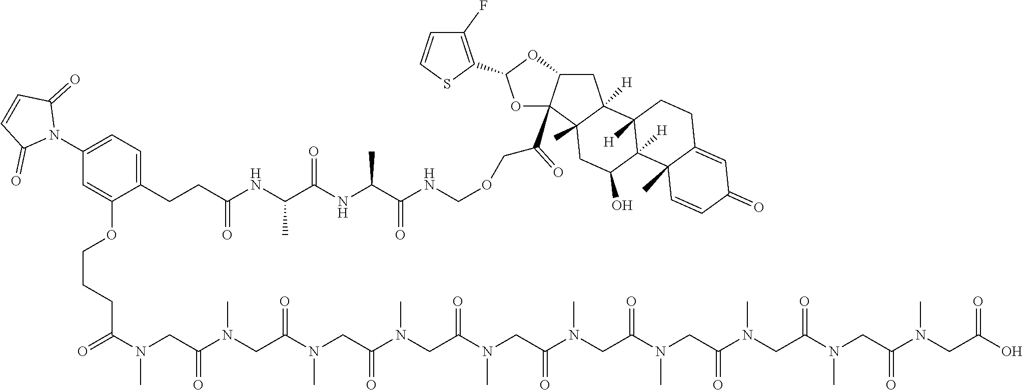 | MS m/z [M + Na]⁺ (ESI): 1722.0; ¹H NMR (400 MHz, DMSO-d₆) δ 8.74-8.60 (m, 1H), 7.57 (s, 1H), 7.32 (d, J = 9.6 Hz, 1H), 7.18 (dd, J = 3.2, 10.4 Hz, 1H), 7.12 (s, 2H), 6.97-6.86 (m, 2H), 6.80-6.71 (m, 1H), 6.21-6.14 (m, 1H), 5.96 (d, J = 18.8 Hz, 2H), 5.73 (s, 1H), 4.87 (d, J = 1.6 Hz, 1H), 4.71-4.50 (m, 4H), 4.31 (s, 6H), 4.22 (d, J = 14.4 Hz, 9H), 2.94-2.75 (m, 35H), 2.43-2.38 (m, 3H), 2.34-2.29 (m, 2H), 2.06 (s, 5H), 1.98-1.88 (m, 3H), 1.82-1.68 (m, 4H), 1.65 (s, 3H), 1.38 (s, 4H), 1.24-1.11 (m, 9H), 0.85 (s, 4H). |
| I-61 | 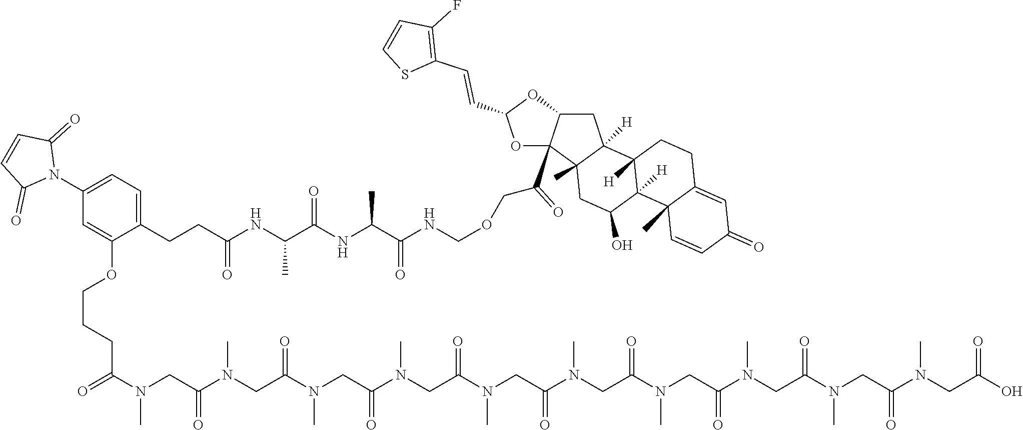 | MS m/z [M − H]⁻ (ESI): 1724.1; ¹H NMR (400 MHz, DMSO-d₆) δ 7.51-7.43 (m, 1H), 7.30 (d, J = 10.0 Hz, 1H), 7.21-7.12 (m, 1H), 7.06 (s, 2H), 6.95 (d, J = 6.0 Hz, 1H), 6.90 (d, J = 16.0 Hz, 1H), 6.84 (d, J = 10.0 Hz, 1H), 6.76- 6.65 (m, 1H), 6.14 (d, J = 9.6 Hz, 1H), 5.91 (s, 1H), 5.87-5.76 (m, 1H), 5.12 (d, J = 6.4 Hz, 1H), 4.84- 4.72 (m, 1H), 4.64-4.48 (m, 3H), 4.31-4.24 (m, 5H), 4.24-4.13 (m, 9H), 4.11-3.92 (m, 11H), 2.97-2.71 (m, 34H), 2.44-2.36 (m, 2H), 2.34- 2.23 (m, 2H), 2.11-1.86 (m, 5H), 1.72 (s, 2H), 1.64-1.52 (m, 3H), 1.35 (s, 3H), 1.22-1.13 (m, 6H), 1.04-0.91 (m, 2H), 0.80 (s, 3H). |
| I-62 | 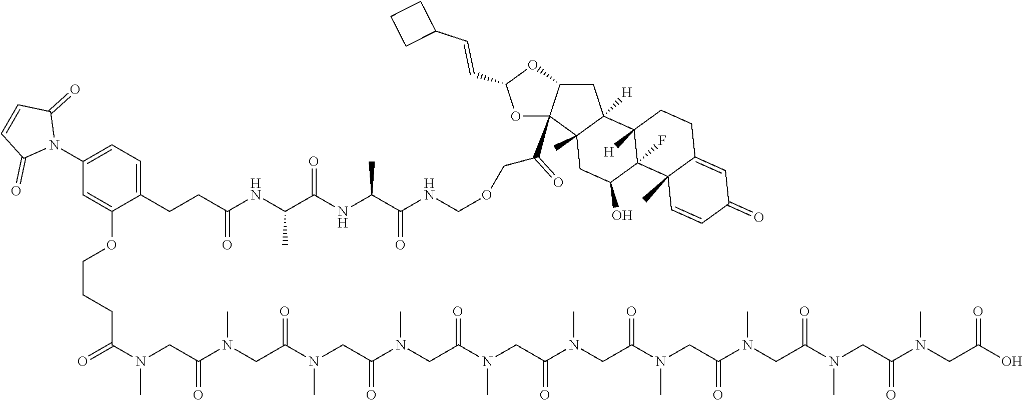 | MS m/z [M − H]⁻ (ESI): 1696.1; ¹H NMR (400 MHz, DMSO-d₆) δ 7.33-7.27 (m, 1H), 7.24-7.17 (m, 1H), 7.10 (s, 2H), 6.88 (d, J = 9.6 Hz, 1H), 6.83-6.72 (m, 1H), 6.28- 6.20 (m, 1H), 6.09-6.00 (m, 2H), 5.27-5.17 (m, 1H), 4.99-4.89 (m, 1H), 4.82-4.74 (m, 1H), 4.67-4.44 (m, 3H), 4.32 (s, 4H), 4.28-4.15 (m, 10H), 4.14-3.90 (m, 12H), 3.03-2.72 (m, 34H), 2.6-2.58 (m, 1H), 2.45-2.29 (m, 4H), 2.07-1.89 (m, 6H), 1.88-1.51 (m, 9H), 1.50- 1.46 (m, 3H), 1.41-1.29 (m, 1H), 1.27-1.15 (m, 6H), 0.82 (s, 3H). |

### Synthetic Example 8

### Synthesis of I-40

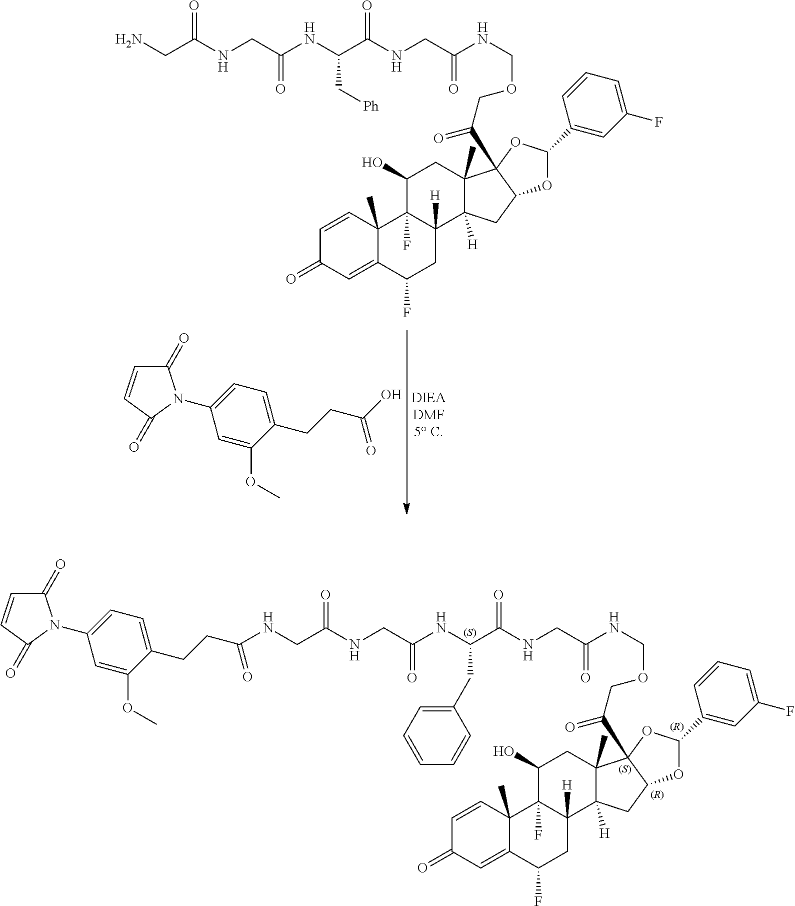

To a stirred mixture of (S)-2-(2-(2-aminoacetamido)acetamido)-N-(2-(((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)amino)-2-oxoethyl)-3-phenylpropanamide in N,N-dimethylformamide with an inert atmosphere of nitrogen, was added N,N-diisopropylethylamine and 3-(4-(2,5-dioxo-2,5-dihydro-1H-pyrrol-1-yl)-2-methoxyphenyl)propanoic acid at 0° C. The resulting mixture was stirred for 3 h at 25° C. The crude product was purified by Prep-HPLC.

MS m/z [M−H]⁻ (ESI): 1121.35; ¹H NMR (300 MHz, Methanol-d₄) δ: 0.98 (s, 3H), 1.48-1.62 (m, 4H), 1.72-1.88 (m, 3H), 2.12-2.27 (m, 1H), 2.28-2.39 (m, 2H), 2.46-2.79 (m, 3H), 2.81-2.92 (m, 2H), 2.93-3.03 (m, 1H), 3.14-3.26 (m, 1H), 3.69-3.99 (m, 9H), 4.21-4.54 (m, 3H), 4.64-4.82 (m, 3H), 4.99-5.09 (m, 1H), 5.38-5.70 (m, 2H), 6.19-6.36 (m, 3H), 6.43-6.59 (m, 1H), 6.89-7.02 (m, 1H), 7.03-7.16 (m, 2H), 7.17-7.42 (m, 10H).

### Synthetic Example 9

### Synthesis of Compound I-38

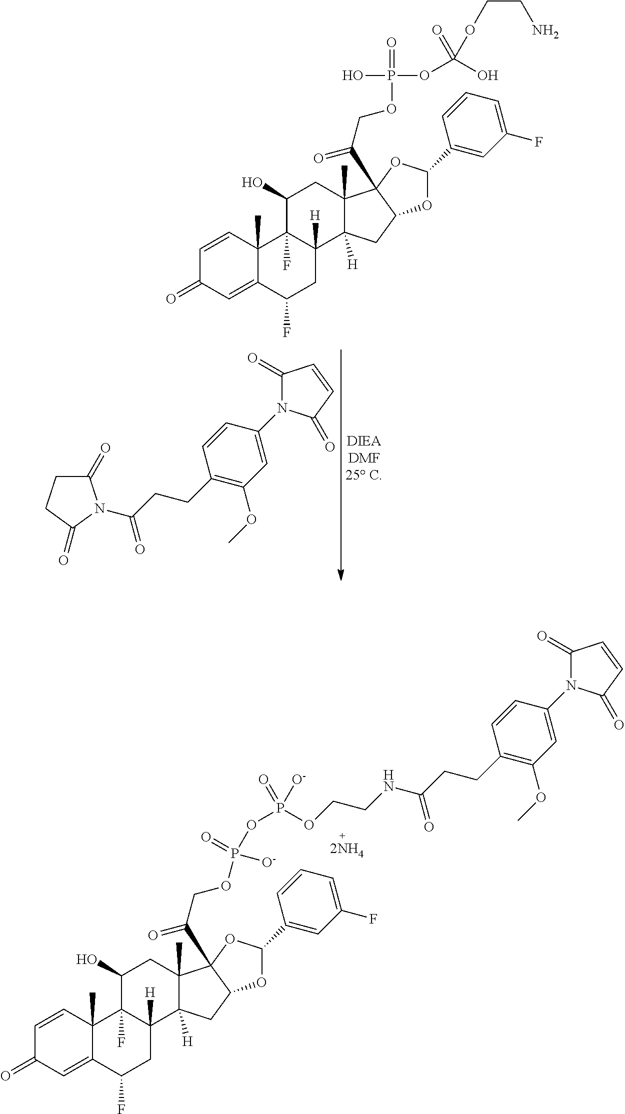

To a solution of the starting material shown in N,N-dimethylformamide with an inert atmosphere of nitrogen, was added 1-(4-(3-(2,5-dioxopyrrolidin-1-yl)-3-oxopropyl)-3-methoxyphenyl)-1H-pyrrole-2,5-dione and N,N-diisopropylethylamine. The resulting solution was stirred for 6 h at 25° C. The crude product was purified by Prep-HPLC with the following conditions (IntelFlash-1): column, XBridge Shield RP18 OBD Column, 19×250 mm, 10 μm; mobile phase, water (with 5.0 mmol/L ammonium bicarbonate) and acetonitrile (22.0% acetonitrile up to 28.0% in 12 min); Detector, UV 254 nm. Desired Compound I-38 was obtained. MS m/z [M−H]⁻ (ESI): 977.30; ¹H NMR (300 MHz, Methanol-d₄) 0.99 (s, 3H), 1.56-1.60 (m, 4H), 1.76-1.78 (m, 2H), 1.91 (d, J=13.4 Hz, 1H), 2.31-2.34 (m, 3H), 2.49-2.53 (m, 2H), 2.84-2.93 (m, 3H), 3.41-3.44 (m, 2H), 3.80 (s, 3H), 4.02-4.04 (m, 2H), 4.30-4.33 (m, 1H), 4.86-4.99 (m, 1H), 5.02-5.05 (m, 2H), 5.50-5.59 (m, 2H), 6.30-6.34 (m, 3H), 6.81-6.93 (m, 3H), 7.01-7.21 (m, 3H), 7.33-7.38 (m, 3H).

The compound in the table below was prepared in a manner similar to the methods and protocols of and preceding Synthetic Example 9 using suitable compounds as starting materials.

| Cmpd. | Structure | MS/¹H NMR |
|---|---|---|
| I-39 | 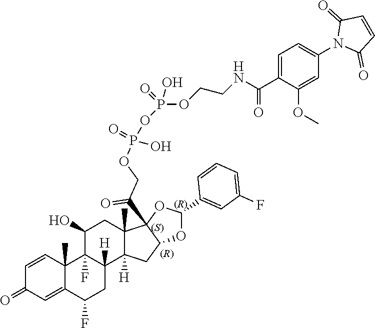 | MS m/z [M − H]⁻ (ESI): 949.10; ¹H NMR (300 MHz, Methanol-d₄) δ: 0.99 (s, 3H), 1.20-1.30 (m, 1H), 1.57 (s, 4H), 1.70-1.78 (m, 2H), 1.90-2.00 (m, 1H), 2.15-2.35 (m, 3H), 2.60- 2.70 (m, 1H), 3.60-3.66 (m, 2H), 3.97-3.99 (m, 3H), 4.13-4.15 (m, 2H), 4.30-4.35 (m, 1H), 5.01-5.05 (m, 2H), 5.50-5.59 (m, 2H), 6.30-6.34 (m, 3H), 6.52-6.60 (m, 1H), 7.16- 7.34 (m, 6H), 7.61 (s, 1H), 7.89-7.92 (m, 1H). |

### Synthetic Example 10

### Synthesis of Compound I-50

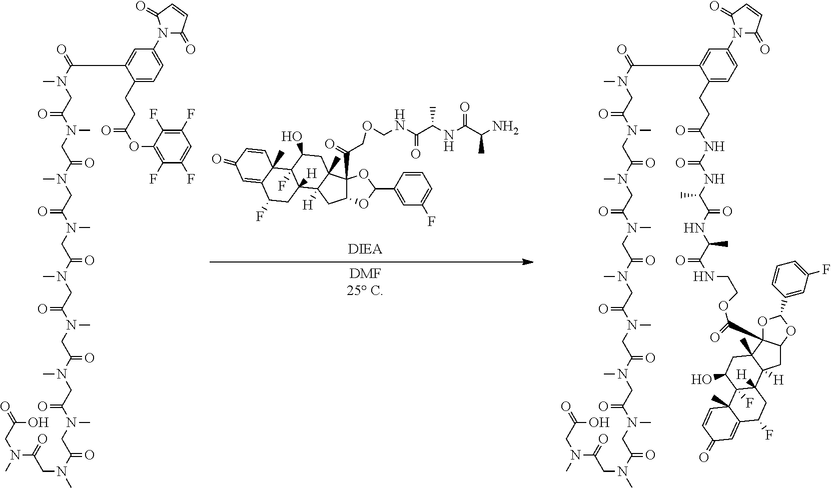

To a solution of (2)-2-amino-N-[(1S)-2-[[2-[(1S,2S,4R,6R,8S,9S,11S,12R,13S,19S)-12,19-difluoro-6-(3-fluorophenyl)-11-hydroxy-9,13-dimethyl-16-oxo-5,7-dioxapentacyclo[10.8.0.02,9.04,8.013,18]icosa-14,17-dien-8-yl]-2-oxo-ethoxy]methylamino]-1-methyl-2-oxo-ethyl]propanamide (30.0 mg, 43.5 μmol, 1.0 eq) in DMF (0.5 mL) was added DIEA (16.8 mg, 130 μmol, 22.7 μL, 3.0 eq) and 2-[[2-[[2-[[2-[[2-[[2-[[2-[[2-[[2-[[2-[[5-(2,5-dioxopyrrol-1-yl)-2-[3-oxo-3-(2,3,5,6-tetrafluorophenoxy)propyl]benzoyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methylamino]acetic acid (49.9 mg, 43.5 μmol, 1.0 eq), and then stirred at 25° C. for 1 hour. The pH of the mixture was adjusted ˜6 with TFA at 0° C., and diluted with addition acetonitrile (0.5 mL). The mixture was purified by (column: C₁₈-1 150×30 mm×5 μm; mobile phase: [water (TFA)-ACN]; B %: 20%-45%, 20 min. The eluent was removed under freeze drying. Compound 2-[[2-[[2-[[2-[[2-[[2-[[2-[[2-[[2-[[2-[[2-[3-[[(1S)-2-[[(1S)-2-[[2-[(1S,2S,4R,6R,8S,9S,11S,12R,13S,19S)-12,19-difluoro-6-(3-fluorophenyl)-11-hydroxy-9,13-dimethyl-16-oxo-5,7-dioxapentacyclo[10.8.0.02,9.04,8.013,18]icosa-14,17-dien-8-yl]-2-oxo-ethoxy]methylamino]-1-methyl-2-oxo-ethyl]amino]-1-methyl-2-oxo-ethyl]amino]-3-oxo-propyl]-5-(2,5-dioxopyrrol-1-yl)benzoyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetic acid (Compound I-50) (11.6 mg, 6.91 μmol, 15.89% yield, 99.61% purity) was obtained as a white solid: ¹H NMR (400 MHz, DMSO-d₆) δ 8.75-8.57 (m, 1H), 8.39-7.95 (m, 1H), 7.50-7.19 (m, 6H), 7.18-6.97 (m, 2H), 6.28 (d, J=10.4 Hz, 1H), 6.12 (s, 1H), 5.75-5.50 (m, 2H), 4.93 (d, J=4.0 Hz, 1H), 4.74-4.50 (m, 3H), 4.43-3.86 (m, 23H), 3.05-2.59 (m, 32H), 2.60-2.54 (m, 1H), 2.43 (s, 2H), 2.35-2.27 (m, 1H), 2.23-2.10 (m, 1H), 2.04-1.91 (m, 1H), 1.80-1.61 (m, 3H), 1.58-1.38 (m, 4H), 1.29-1.09 (m, 6H), 0.85 (s, 3H); LC/MS [M−H] 1669.7 (calculated); LC/MS [M−H] 1670.0 (observed).

The compounds in the table below were prepared in a manner similar to the methods and protocols of and preceding Synthetic Example 10 using suitable compounds as starting materials.

| Cmpd. | Structure | MS / 1H NMR |
|---|---|---|
| I-52 | 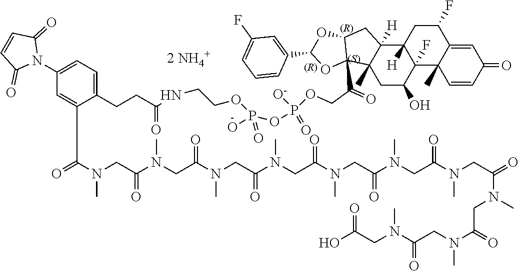 | MS m/z [M − H]⁻ (ESI): 1701.5896; ¹H NMR (DMSO-d₆, 400 MHz) δ 7.46-7.38 (m, 2H), 7.31- 7.14 (m, 6H), 7.08-7.01 (m, 2H), 6.26 (d, J = 9.6 Hz, 1H), 6.13 (s, 1H), 5.70-5.48 (m, 2H), 5.07- 4.92 (m, 2H), 4.73 (dd, J = 8.8 Hz, 18.0 Hz, 1H), 4.32-3.91 (m, 25H), 3.00-2.74 (m, 34H), 2.43 (t, J = 7.6 Hz, 2H), 2.25- 2.15 (m, 1H), 2.04 (d, J = 14.0 Hz, 1H), 1.89 (d, J = 14.0 Hz, 1H), 1.72 (d, J = 8.0 Hz, 2H), 1.50 |
| | | (s, 4H), 0.92 (s, 3H) |
| I-51 | 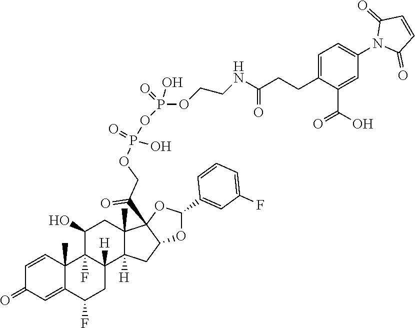 | MS m/z [M + H]⁺ (ESI): 993.4; ¹H NMR (DMSO-d₆, 400 MHz) δ 7.73 (d, J = 1.6 Hz, 1H), 7.46-7.38 (m, 3H), 7.31- 7.19 (m, 4H), 7.12 (s, 2H), 6.27 (dd, J = 1.6, 10.0 Hz, 1H), 6.12 (s, 1H), 5.74-5.52 (m, 2H), 5.05 (dd, J = 9.6, 18.8 Hz, 1H), 4.94 (d, J = 4.0 Hz, 1H), 4.74 (dd, J = 8.4, 18.8 Hz, 1H), 4.19 (d, J = 8.8 Hz, 1H), 3.91 (q, J = 6.0 Hz, 2H), 3.25 (t, J = 5.6 Hz, 2H), 3.13 (t, J = 8.0 Hz, 2H), 2.45- 2.38 (m, 2H), 2.31-2.26 (m, 1H), 2.20-2.12 (m, 1H), 2.02-1.92 (m, 1H), 1.82 (d, J = 14.0 Hz, |
| | | 1H), 1.77-1.66 (m, 2H), |
| | | 1.47 (s, 4H), 0.88 (s, 3H) |
| I-53 | 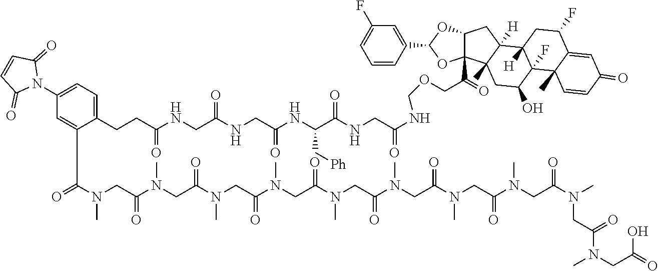 | MS m/z [M − H]⁻ (ESI): 1846.1; ¹H NMR (400 MHz, DMSO-d₆) δ 8.58 (s, 1H), 8.39-8.28 (m, 1H), 8.21-8.03 (m, 2H), 8.20-8.03 (m, 2H), 7.33- 7.07 (m, 11H), 6.25 (d, J = 9.6 Hz, 1H), 6.11 (s, 1H), 5.75-5.53 (m, 2H), 4.94 (d, J = 4.8 Hz, 1H), 4.72-4.56 (m, 3H), 4.50- 3.85 (m, 22H), 3.78-3.65 (m, 6H), 3.11-2.68 (m, 30H), 2.54-2.52 (m, 1H), 2.27-2.25 (m, 1H), 2.23- 2.10 (m, 2H), 1.99 (d, J = 12.8 Hz, 1H), 1.82- |
| | | 1.63 (m, 3H), 1.48 (s, |
| | | 4H), 0.86 (s, 3H). |
| I-55 | 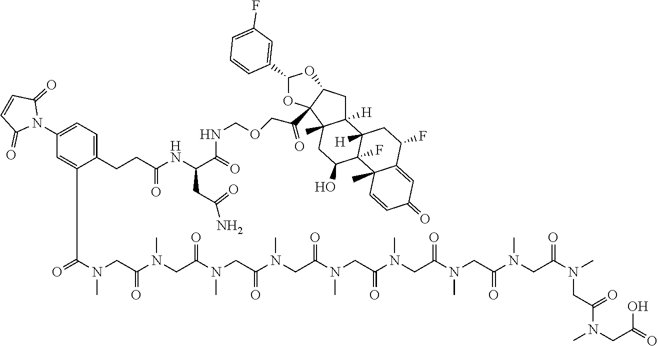 | MS m/z [M + Na]⁺ (ESI): 1665.8; ¹H NMR (400 MHz, DMSO-d₆) δ 8.72- 8.59 (m, 1H), 7.45-7.35 (m, 1H), 7.34-7.10 (m, 7H), 7.02 (s, 2H), 6.31- 6.23 (m, 1H), 6.13 (s, 1H), 5.69-5.60 (m, 1H), 5.57-5.52 (m, 1H), 4.89 (s, 1H), 4.63-4.50 (m, 4H), 4.32-4.15 (m, 12H), 2.98-2.67 (m, 35H), 2.44-2.28 (m, 5H), 2.18- 2.05 (m, 1H), 1.73-1.63 (m, 3H), 1.46-143 (m, 4H), 0.82 (s, 3H) |

### Synthetic Example 11

### Synthesis of Compound I-57

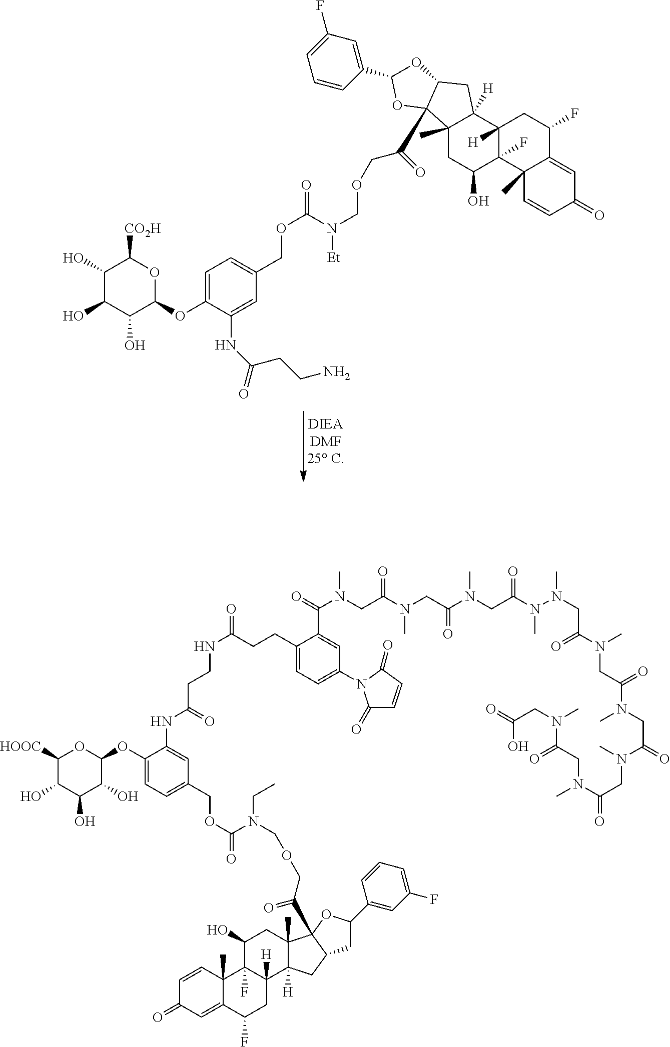

To a solution of (2S,3S,4S,5R,6S)-6-[2-(3-aminopropanoylamino)-4-[[[2-[(1S,2S,4R,6R,8S,9S,11S,12R,13S,19S)-12,19-difluoro-6-(3-fluorophenyl)-11-hydroxy-9,13-dimethyl-16-oxo-5,7-dioxapentacyclo[10.8.0.02,9.04,8.013,18]icosa-14,17-dien-8-yl]-2-oxo-ethoxy]methyl-ethyl-carbamoyl]oxymethyl]phenoxy]-3,4,5-trihydroxy-tetrahydropyran-2-carboxylic acid (30.0 mg, 30.3 μmol, 1.0 eq) in DMF (0.5 mL) was added DIEA (11.7 mg, 91.1 μmol, 15.8 μL, 3.0 eq) and 2-[[2-[[2-[[2-[[2-[[2-[[2-[[2-[[2-[[2-[[5-(2,5-dioxopyrrol-1-yl)-2-[3-oxo-3-(2,3,5,6-tetrafluorophenoxy)propyl]benzoyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methylamino]acetyl]-methyl-amino]acetyl]-methyl-amino]acetic acid (34.8 mg, 30.3 μmol, 1.0 eq.), and then stirred at 25° C. for 1 hour. The pH of the reaction mixture was adjusted to ˜6 with FA at 0° C., and diluted with addition MeCN (0.5 mL). The mixture was purified by prep-HPLC (column: Phenomenex Luna C₁₈ 200×40 mm×10 μm; mobilephase: [water (FA)-ACN]; B %: 15%-50%, 8 min). The eluent was removed under freeze drying. Compound (2S,3S,4S,5R,6S)-6-[2-[3-[3-[2-[[2-[[2-[[2-[[2-[[2-[[2-[[2-[[2-[[2-[carboxymethyl(methyl)amino]-2-oxo-ethyl]-methyl-amino]-2-oxo-ethyl]-methyl-amino]-2-oxo-ethyl]-methyl-amino]-2-oxo-ethyl]-methyl-amino]-2-oxo-ethyl]-methylamino]-2-oxo-ethyl]-methyl-amino]-2-oxo-ethyl]-methyl-amino]-2-oxo-ethyl]-methyl-amino]-2-oxo-ethyl]-methyl-carbamoyl]-4-(2,5-dioxopyrrol-1-yl)phenyl]propanoylamino]propanoylamino]-4-[[[2-[(1S,2S,4R,6R,8S,9S,11S,12R,13S,19S)-12,19-difluoro-6-(3-fluorophenyl)-11-hydroxy-9,13-dimethyl-16-oxo-5,7-dioxapentacyclo[10.8.0.02,9.04,8.013,18]icosa-14,17-dien-8-yl]-2-oxo-ethoxy]methyl-ethyl-carbamoyl]oxymethyl]phenoxy]-3,4,5-trihydroxy-tetrahydropyran-2-carboxylic acid Compound I-57 (26.6 mg, 12.3 μmol, 40.77% yield, 91.69% purity) was obtained as a white solid: ¹H NMR (400 MHz, DMSO-d₆) δ 8.16 (s, 1H), 7.52-6.93 (m, 11H), 6.29 (dd, J=1.6, 10.0 Hz, 1H), 6.12 (s, 1H), 5.75-5.47 (m, 2H), 5.09-4.90 (m, 3H), 4.89-4.59 (m, 4H), 4.44-3.77 (m, 22H), 3.39-3.25 (m, 7H), 3.03-2.63 (m, 31H), 2.62-2.53 (m, 4H), 2.44-2.26 (m, 4H), 2.23-2.12 (m, 1H), 2.02-1.97 (m, 1H), 1.77-1.65 (m, 3H), 1.52-1.45 (m, 4H), 1.15-1.02 (m, 3H), 0.86 (s, 3H); LC/MS [M+H] 1969.8 (calculated); LC/MS [M+H] 1969.9 (observed).

The compound in the table below was prepared in a manner similar to the methods and protocols of and preceding Synthetic Example 11 using suitable compounds as starting materials.

| Cmpd. | Structure/Name | MS/¹H NMR |
|---|---|---|
| I-42 | 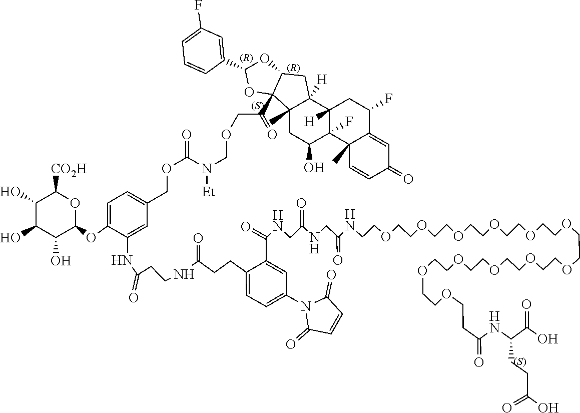 | MS m/z [M/2 − H]⁻ (ESI): 1050.05; ¹H NMR (300 MHz, Methanol- d₄) δ: 0.71-0.93 (m, 4H), 1.08 (t, J = 7.1 Hz, 4H), 1.17-1.31 (m, 1H), 1.47 (s, 4H), 1.67 (s, 3H), 1.76-1.95 (m, 2H), 2.00-2.17 (m, 2H), 2.19-2.33 (m, 4H), 2.32- 2.55 (m, 6H), 3.04 (t, J = 7.3 Hz, 2H), 3.25-3.39 (m, 6H), 3.47- 3.57 (m, 42H), 3.59-3.72 (m, 4H), 3.82 (s, 2H), 4.00 (s, 2H), 4.15-4.37 (m, 4H), 4.63 (s, 2H), 4.76-5.07 (m, 6H), 5.41 (q, J = 23.0, 21.6 Hz, 3H), 6.24 (d, J = 19.0 Hz, 3H), 6.80 (s, 2H), 6.88- 7.09 (m, 3H), 7.11-7.39 (m, 7H), 8.09 (s, 1H) |

### Conjugation Example 1

### Preparation of Antibody Conjugates

A targeting moiety (e.g., a mAb at 3-8 mg/mL in PBS) is exchanged into HEPES (100 mM, pH 7.0, 1 mM DTPA) via molecular weight cut-off centrifugal filtration (Millipore, 30 kDa). The resultant solution is transferred to a tared 50 mL conical tube. The protein concentration is determined to be 3-8 mg/mL by A280. To the protein solution is added TCEP (2.0-4.0 equivalents, 1 mM stock) at room temperature and the resultant mixture is incubated at 37° C. for 30-90 minutes, with gentle shaking. Upon being cooled to room temperature, a stir bar is added to the reaction tube. Next, the compound of Structure (I) (5.0-10.0 equivalents, 10 mM DMSO) is added dropwise. The resultant reaction mixture is allowed to stir at ambient temperature for 30-60 minutes, at which point N-ethyl maleimide (3.0 equivalents, 100 mM DMA) is added. After an additional 15 minutes of stirring, N-acetylcysteine (6.0-11.0 equivalents, 50 mM HEPES) is added. The crude antibody conjugate is then exchanged into PBS and purified by preparative SEC (e.g. HiLoad 26/600, Superdex 200 pg) using PBS as the mobile phase. The pure fractions are concentrated via molecular weight cut-off centrifugal filtration (Millipore, 30 kDa), sterile filtered, and transferred to 15 mL conical tubes.

### Conjugation Example 2

### Preparation of Antibody Conjugate

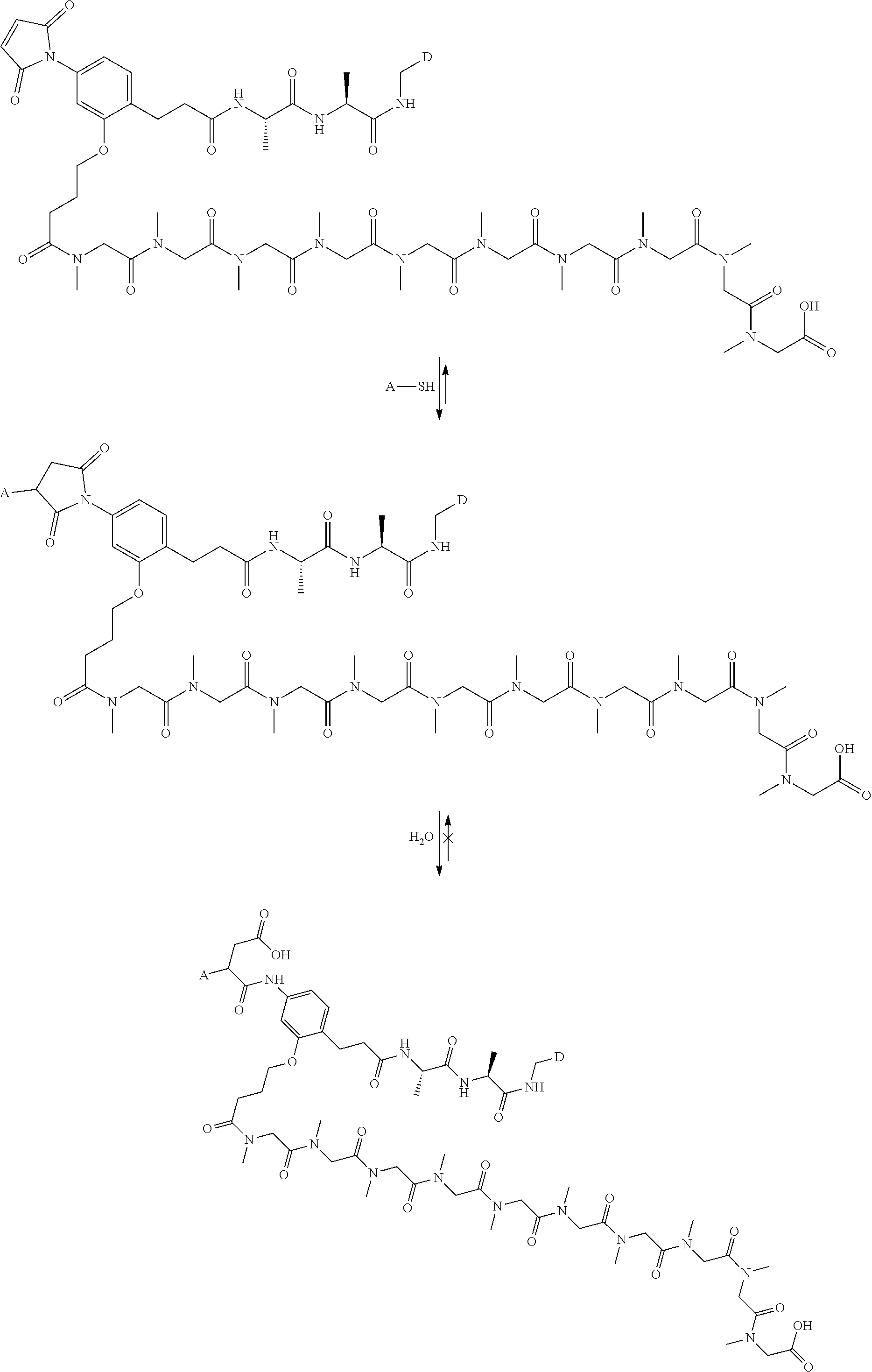

The conjugation reaction above is illustrative of a method and reaction scheme described herein (see, e.g., General Conjugation Scheme and Conjugation Example 1).

### Biological Example 1

### Human PBMC TNF-alpha Production Induced by anti-HER2 STING and anti-Nectin-4 TLR8 Agonist Conjugates in the Presence of HER2 or Nectin-4 Expressing Tumor Cell Lines

Production of TNF-α from peripheral blood mononuclear cells (PBMCs) co-cultured with either HER2 or Nectin-4 expressing tumor cell lines in the presence of anti-HER2-STING (e.g., conjugate II-12) or anti-Nectin-4-TLR8 (e.g., conjugate II-1, etc.) agonist conjugates was examined. Briefly, PBMCs were isolated from normal human donor peripheral blood using SepMate™-50 PBMC Isolation Tubes (STEMCELL Technologies) according to manufacturer's instructions from normal, healthy human donors. Isolated PBMCs were cultured with the HER2 expressing tumor cell line SK-BR-3 (ATCC) or the HER2 negative cell line MDA-MB-468 (ATCC) at a 5:1 ratio in the presence of titrated concentrations of anti-HER2-STING antibody conjugates. For Nectin-4-TLR8 conjugates, Isolated PBMCs were cultured with the Nectin-4 expressing tumor cell line MDA-MB-175-VII (ATCC) or the Nectin-4 negative cell line HEK-293 (ATCC) at a 5:1 ratio in the presence of titrated concentrations of anti-Nectin-4-TLR8 antibody conjugates or the unconjugated anti-Nectin-4 antibody control. After 24 hours, the cell-free supernatants were collected and stored at −80° C. prior to analysis. TNF-α levels in the cell-free supernatants were quantified using the TNF-α (human) AlphaLISA® Detection Kit (Perkin Elmer) according to manufacturer's instructions.

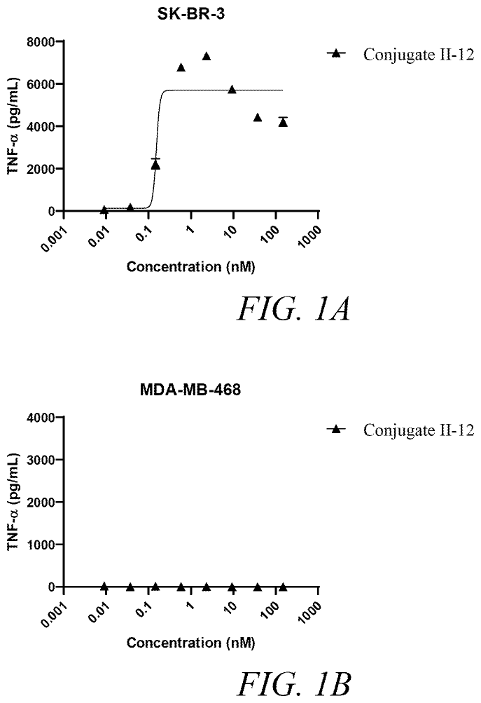

FIG. 1 shows that conjugate II-12 induced TNF-α production in a dose-dependent manner from human PBMCs in the presence of the HER2 expressing SK-BR-3 tumor cell line (FIG. 1A), but not in the presence of MDA-MB-468 cells lacking expression of HER2 (FIG. 1B).

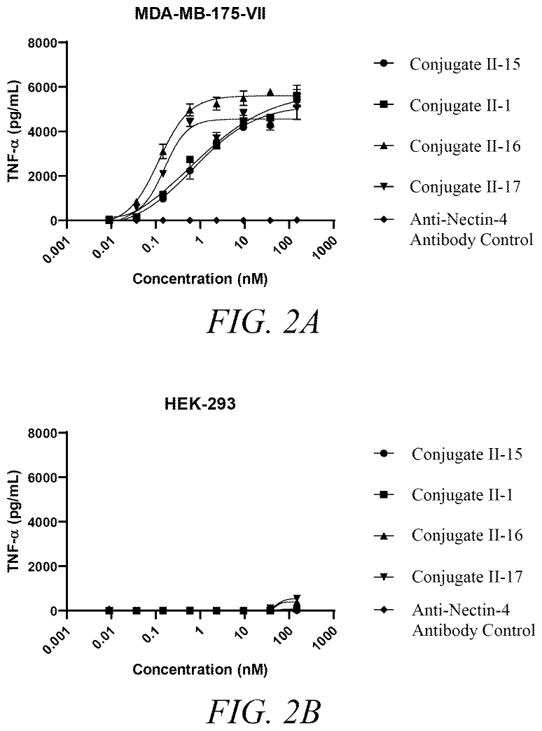

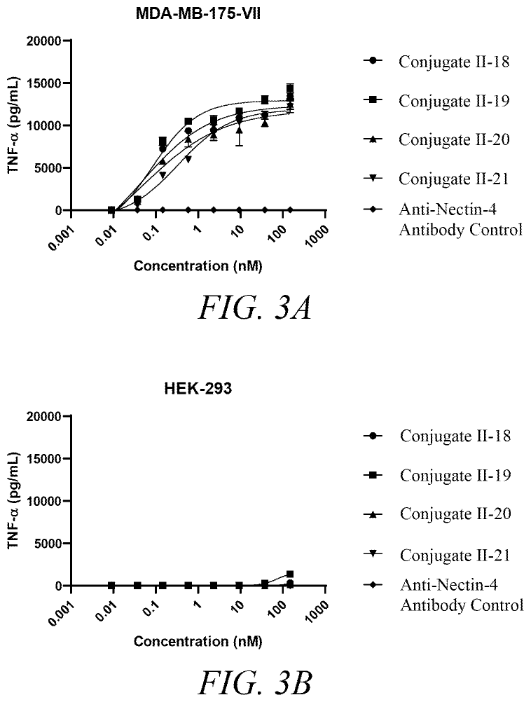

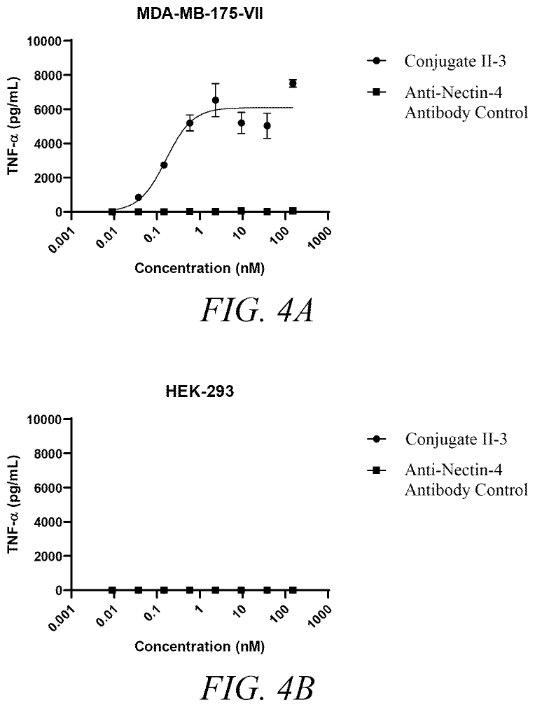

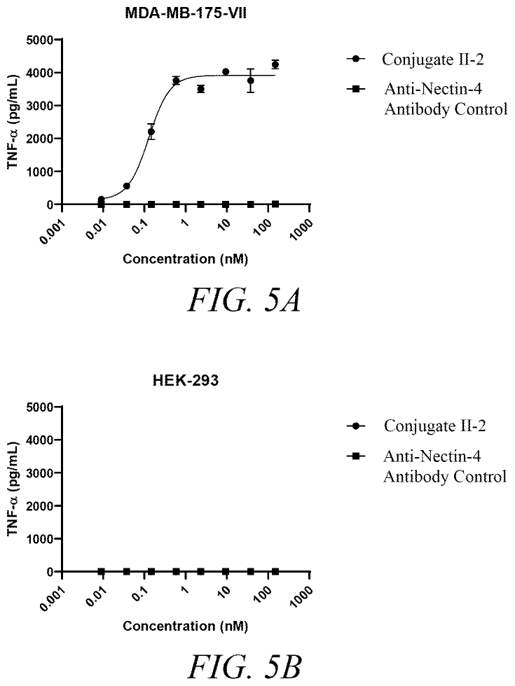

FIGS. 2-5 shows that the anti-Nectin-4-TLR8 agonist conjugates II-1, II-2, II-3, II-15, II-16, II-17, II-18, II-19, II-20, and II-21 induced TNF-α production in a dose-dependent manner from human PBMCs in the presence of the Nectin-4 expressing MDA-MB-175-VII tumor cell line (FIGS. 2A, 3A, 4A, and 5A), but not in the presence of HEK-293 cells lacking expression of Nectin-4 (FIGS. 2B, 3B, 4B, and 5B). TNF-α production by PBMCs was not induced in the presence of the Nectin-4 expressing tumor cell line with unconjugated anti-Nectin-4 antibody, which indicates that TLR8 agonism is needed for TNF-α release. Furthermore, none of the conjugated or unconjugated antibodies stimulated TNF-α production from PBMCs in the absence of Nectin-4-expressing tumor cells indicating that the activity is dependent upon Nectin-4 expression. Low level PBMC TNF-α production was observed with the Nectin-4 negative HEK-293 cell line only in cultures containing the highest concentrations of anti-Nectin-4-TLR8 conjugates tested (see, e.g., FIGS. 2B and 3B).

Table A represents the compiled EC₅₀ values for the tested conjugates demonstrating potencies below 1.0 nM for all conjugates.

TABLE A
Representative biological data for
selected conjugates of Structure (II)

| Conjugate | EC₅₀ | Target | Agonist |
|---|---|---|---|
| II-12 | 0.15 nM | HER2 | STING |
| II-15 | 0.71 nM | Nectin-4 | TLR8 |
| II-1 | 0.62 nM | Nectin-4 | TLR8 |
| II-16 | 0.12 nM | Nectin-4 | TLR8 |
| II-17 | 0.16 nM | Nectin-4 | TLR8 |
| II-18 | 0.05 nM | Nectin-4 | TLR8 |
| II-19 | 0.08 nM | Nectin-4 | TLR8 |
| II-20 | 0.05 nM | Nectin-4 | TLR8 |
| II-21 | 0.34 nM | Nectin-4 | TLR8 |
| II-3 | 0.16 nM | Nectin-4 | TLR8 |
| II-2 | 0.13 nM | Nectin-4 | TLR8 |

### Biological Example 2

### Serum Stability of Antibody Conjugate Linker-Payloads

The in vitro stability of conjugated linker-payloads in serum from multiple species was characterized for antibody conjugates based on measurements of drug-to-antibody ratio (DAR) values over time. Briefly, an antibody conjugate was recovered from serum by affinity capture with either anti-human IgG, HER2, or Nectin-4 extracellular domain reagents coupled to magnetic beads. The enriched antibody conjugate was reduced prior to LC-MS analysis for accurate mass and peak area characterization of light and heavy chains, with or without conjugated linker-payload. DAR values were calculated from the weighted proportion of drug-loaded light and heavy chains using the manufacturer's custom method (Waters). Table B represents the compiled stability of each antibody conjugate determined as the % DAR initial remaining after each time-point. Conjugate II-12 and conjugate II-15 were stable in mouse and monkey serum, retaining at least 80% initial DAR values after 168 hours, indicating favorable stability for in vivo models.

### Experimental Materials and Methods

The antibody conjugate was diluted to a final concentration of 1 μM in sterile, 0.2 micron-filtered serum (BioIVT, Westbury, NY) from cynomolgus monkey, BALB/c mouse and/or human. An initial time-point (time 0) aliquot was collected and immediately frozen on dry ice. The remaining sample volume was incubated at 37° C. 5% CO₂ and aliquots were collected at 48, 96 and 168 hours for analysis. All collected samples were stored in a freezer set at −20° C. prior to analysis.

Serum samples were thawed at room temperature, placed on wet ice and then diluted 1:10 in 1×PBS. The antibody conjugate was recovered by affinity capture using either a commercially available biotinylated anti-human IgG llama antibody fragment or proprietary biotinylated HER2 or Nectin-4 protein ECD reagent bound to Streptavidin Magnetic Sepharose beads (Cytiva Life Sciences, Marlborough, MA). After collecting beads and washing extensively with PBST and PBS buffer consecutively to remove non-specifically bound protein, the antibody conjugate was eluted with 30 mM HCl. The eluate was neutralized, reduced with 50 mM DTT for 30 min at 37° C. then diluted to 10% acetonitrile −1% formic acid.

For each sample, a Waters Acquity H-class UPLC system connected to a Waters Xevo G2-XS QTOF was used to separate light chains (LC) and heavy chains (HC) on a Waters Protein BEH C₄ column (300 Å, 1.7 μm, 2.1 mm×50 mm) at 80° C. using a reverse-phase gradient of water-0.1% formic acid and acetonitrile-0.1% formic acid. Analytes were detected by LC-MS analysis using positive mode electrospray ionization and mass spectra were acquired. The extracted ion chromatograms were deconvoluted to neutral mass by Maximum Entropy method and peak areas were calculated. Weighted DAR values were determined by first summing the extracted peak areas for unconjugated and linker-payload conjugated LC or HC components, calculating the proportion of each component to the total response, and then weighting each % total response by drug load (i.e., 0, 1, 2 or 3 linker-payloads) for the antibody conjugate.

TABLE B
Serum stability of representative conjugates of Structure (II)

| Conjugate | % initial DAR retained | | | % initial DAR retained | | | % initial DAR retained | | |
|---|---|---|---|---|---|---|---|---|---|
| of Structure | in mouse serum | | | in monkey serum | | | in human serum | | |
| (II) | 48 hr | 96 hr | 168 hr | 48 hr | 96 hr | 168 hr | 48 hr | 96 hr | 168 hr |
| II-12 | 84 | 86 | 83 | 85 | 82 | 81 | TBD | TBD | TBD |
| II-15 | 89 | 86 | 81 | 98 | 93 | 85 | TBD | TBD | TBD |
| II-1 | 95 | 91 | 86 | 94 | 91 | 86 | TBD | TBD | TBD |
| II-16 | 78 | 70 | 56 | 89 | 85 | 84 | TBD | TBD | TBD |
| II-17 | 89 | 85 | 76 | 95 | 86 | 83 | TBD | TBD | TBD |
| II-18 | 90 | 84 | 77 | 91 | 90 | 84 | TBD | TBD | TBD |
| II-19 | 78 | 78 | 64 | 89 | 87 | 82 | TBD | TBD | TBD |
| II-20 | 85 | 80 | 70 | 88 | 85 | 82 | TBD | TBD | TBD |
| II-21 | 87 | 86 | 72 | 88 | 87 | 80 | TBD | TBD | TBD |
| II-3 | 91 | 88 | 81 | 84 | 81 | 75 | TBD | TBD | TBD |

TBD = to be determined

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by this disclosure.

1-132. (canceled)

133. A compound having the following Structure (Ia): 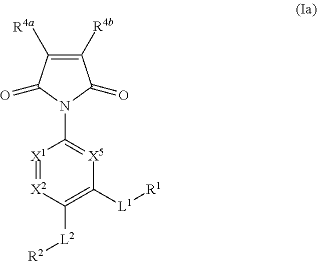
wherein:
R¹ and R² each independently comprise one or more moieties selected from:
i) an amino acid element that comprises 1, 2, 3, 4, or 5 amino acids selected from the group consisting of glycine, alanine, serine, threonine, cysteine, valine, leucine, isoleucine, methionine, proline, phenylalanine, tyrosine, tryptophan, aspartic acid, glutamic acid, asparagine, glutamine, histidine, lysine, arginine, sarconsine, and beta-alanine;
ii) a charged element comprising one or more charged amino acid, one or more carboxylic acid, one or more sulfonic acid, one or more sulfonamide, one or more sulfate, one or more phosphate, one or more quaternary amine, one or more sulfamide, one or more sulfinimide, or combinations thereof;
iii) a heteroalkylene element comprising one of the following structures: 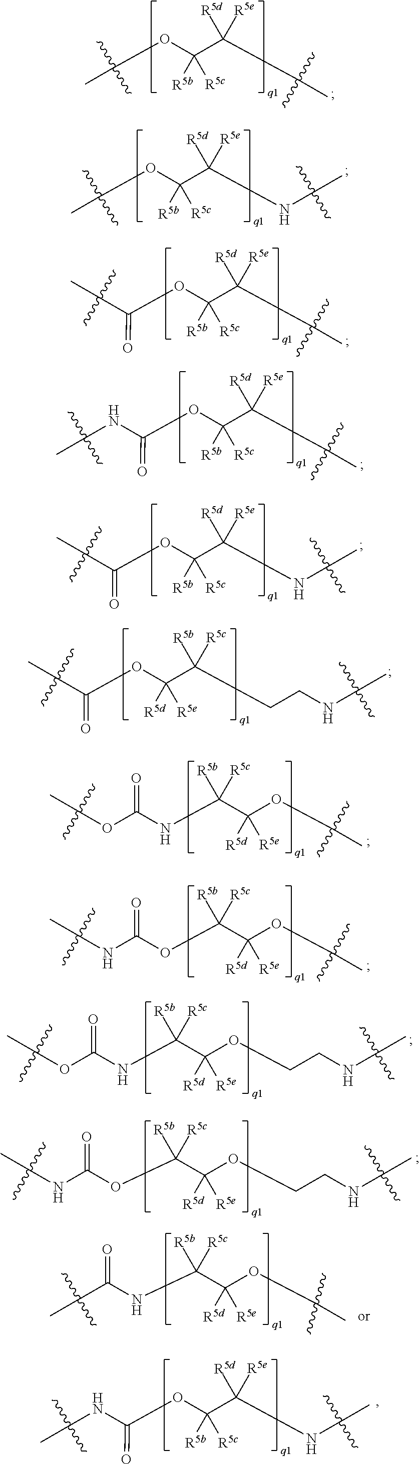
wherein:
each occurrence of R^{5b}, R^{5c} R^{5d}, and R^{5e} is independently selected from the group consisting of hydrogen, deuterium, alkyl, haloalkyl, halo, alkoxy, haloalkoxy, amino, hydroxyl, cyano, nitro, thiol, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, and —P(O)₃H,
each occurrence of q1 is, independently an integer from 1-24;
iv) a hydrophilic element comprising polyethylene glycol, polysarcosine, cyclodextrin, c-glycosides, or combinations thereof;
v) a trigger element comprising a phosphate, a diphosphate, a triphosphate, a glucuronide, or two or more amino acids selected from the group consisting of valine, citrulline, alanine, glycine, phenylalanine, lysine, or combinations thereof;
vi) an immolative element comprising para-aminobenzyloxycarbonyl, an aminal, a hydrazine, a disulfide, an amide, an ester, a hydrazine, a diester, a β-glucuronide, a double bond, a triple bond, an ether bond, a ketone, a diol, a cyano, a nitro, a quaternary amine, or combinations thereof;
vii) a polar cap comprising —OH, —NH₂, —C(═O)OH, —S(O)₃, or —OP(O)₃; and
viii) a payload that is a glucocorticoid receptor agonist, a chemotherapeutic, a cytotoxic agent, or a myeloid cell agonist, provided that at least one of R¹ and R² comprises the payload;
R^{4a} and R^{4b} are each independently hydrogen, deuterium, halo, or —S—R^{4c} wherein R^{4c} is substituted or unsubstituted C₆-C₁₀ aryl or substituted or unsubstituted 5-12 membered heteroaryl;
L¹ and L² independently have one of the following structures: 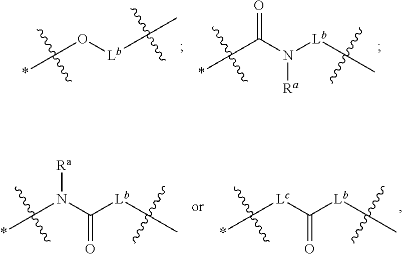
wherein:
* indicates a direct bond to the ring carbon as indicated in Structure (Ia);
R^{a} is hydrogen or alkyl;
each occurrence of L^{b} is independently a direct bond, an optionally substituted alkylene linker, an optionally substituted heteroalkylene linker, a heteroatomic linker, or combinations thereof;
each occurrence of L^{c} is independently an optionally substituted alkylene linker; and
X¹, X², and X⁵ are independently CH or CF;
as a stereoisomer, enantiomer, or tautomer thereof or a mixture thereof; or a pharmaceutically acceptable salt, solvate or prodrug thereof.

134. The compound of claim 133, wherein X¹ is CF, X² is CH, and X⁵ is CH as a stereoisomer, enantiomer, or tautomer thereof or a mixture thereof, or a pharmaceutically acceptable salt, solvate or prodrug thereof.

135. The compound of claim 133, wherein X⁵ is CF, X¹ is CH, and X² is CH, as a stereoisomer, enantiomer, or tautomer thereof or a mixture thereof; or a pharmaceutically acceptable salt, solvate or prodrug thereof.

136. The compound of claim 133, wherein the amino acid element has one of the following structures: 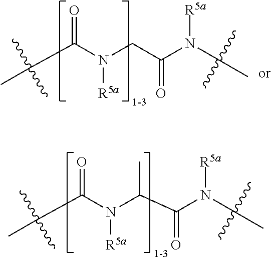
wherein:
each occurrence of R^{5a} is independently hydrogen, alkyl, hydroxyalkyl, or alkoxyalkyl, as a stereoisomer, enantiomer, or tautomer thereof or a mixture thereof; or a pharmaceutically acceptable salt, solvate or prodrug thereof.

137. The compound of claim 133, wherein the amino acid element has the following structure: 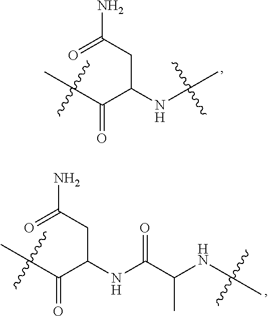 [SMILES: *NC(CC(N)=O)C(*)=O ; *NC(C)C(=O)NC(CC(N)=O)C(*)=O]
as a stereoisomer, enantiomer, or tautomer thereof or a mixture thereof; or a pharmaceutically acceptable salt, solvate or prodrug thereof.

138. The compound of claim 133, wherein the heteroalkylene element comprises one of the following structures: 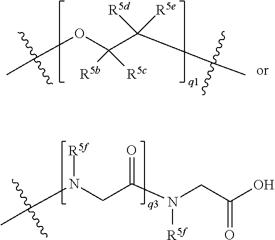
wherein:
each occurrence of R^{5b}, R^{5c} R^{5d}, and R^{5e} is independently hydrogen; and
each occurrence of R^{5f} is independently hydrogen or alkyl;
each occurrence of q3 is independently an integer from 5-15;
each occurrence of q1 is, independently an integer from 1-24, as a stereoisomer, enantiomer, or tautomer thereof or a mixture thereof; or a pharmaceutically acceptable salt, solvate or prodrug thereof.

139. The compound of claim 133, wherein the hydrophilic element comprises one of the following structures: 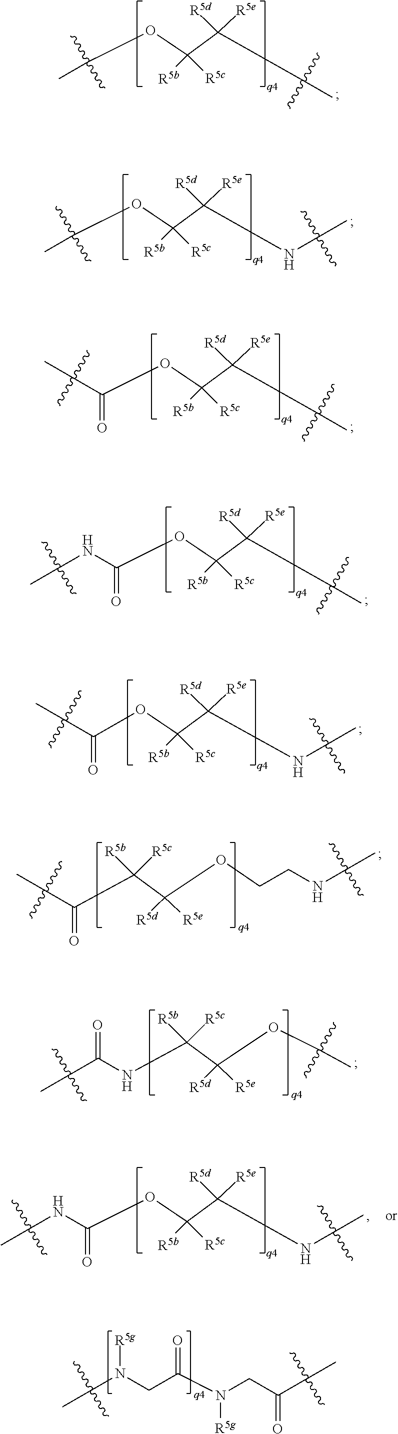
wherein:
each occurrence of R^{5b}, R^{5c} R^{5d}, and R^{5e} is independently selected from the group consisting of hydrogen, deuterium, alkyl, haloalkyl, halo, alkoxy, haloalkoxy, amino, hydroxyl, cyano, nitro, thiol, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, and —P(O)₃H,
each occurrence of R^{5g} is independently hydrogen, alkyl, hydroxyalkyl, or alkoxyalkyl; and
each occurrence of q4 is, independently an integer from 1-24, as a stereoisomer, enantiomer, or tautomer thereof or a mixture thereof; or a pharmaceutically acceptable salt, solvate or prodrug thereof.

140. The compound of claim 133, wherein the hydrophilic element comprises one of the following structures: 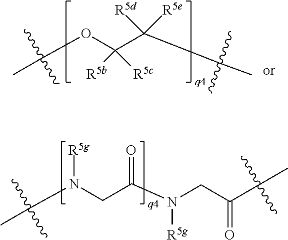
wherein:
each occurrence of R^{5b}, R^{5c} R^{5d}, and R^{5e} are hydrogen;
each occurrence of R^{5g} is independently alkyl; and
each occurrence of q4 is, independently an integer from 1-24, as a stereoisomer, enantiomer, or tautomer thereof or a mixture thereof; or a pharmaceutically acceptable salt, solvate or prodrug thereof.

141. The compound of claim 133, wherein the trigger element comprises a sequence of amino acids selected from the group consisting of alanine-alanine, valine-citrulline, valine-alanine, glycine-glycine-phenylalanine-glycine, and combinations thereof as a stereoisomer, enantiomer, or tautomer thereof or a mixture thereof; or a pharmaceutically acceptable salt, solvate or prodrug thereof.

142. The compound of claim 133, wherein the trigger element has one of the following structures: 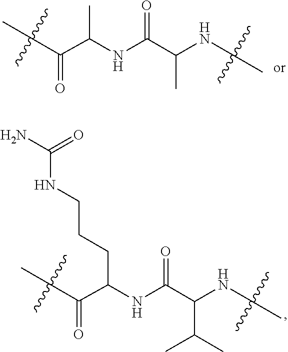 [SMILES: *NC(C)C(=O)NC(C)C(*)=O ; *NC(C(=O)NC(CCCNC(N)=O)C(*)=O)C(C)C]
as a stereoisomer, enantiomer, or tautomer thereof or a mixture thereof; or a pharmaceutically acceptable salt, solvate or prodrug thereof.

143. The compound of claim 133, wherein the immolative element comprises:
(i) one of the following structures: 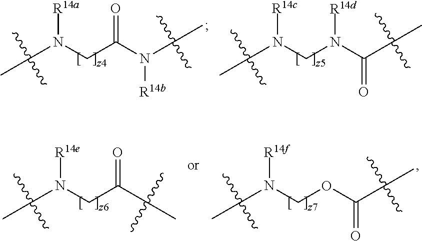
wherein:
R^{14a}, R^{14b}, R^{14c}, R^{14d}, R^{14e}, and R^{14f} are each independently hydrogen, alkyl, hydroxyalkyl, or alkoxyalkyl;
z4, z5, z6, and z7 are each independently 1, 2, 3, 4, 5, or 6;
(ii) the following structure: 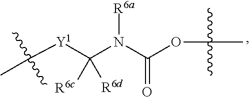
wherein:
R^{6a}, R^{6b}, R^{6c}, and R^{6d} are independently hydrogen, an optionally substituted alkyl, an optionally substituted aryl, or optionally substituted heteroaryl, or
R^{6a} and R^{6c} together with the nitrogen and carbon atoms to which they are attached form azetidinyl, pyrrolodinyl, piperidinyl, or homopiperidinyl and R^{6d} is hydrogen; and
Y¹ is —O—, —S—, or —NR^{6b}—; or
(iii) one of the following structures: 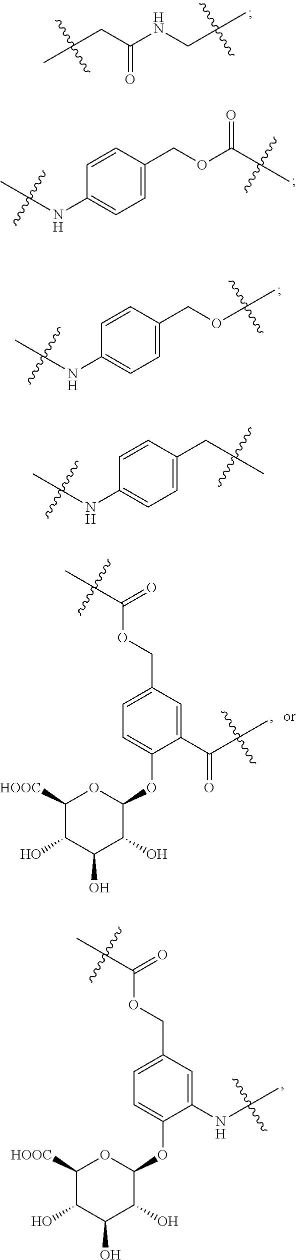
as a stereoisomer, enantiomer, or tautomer thereof or a mixture thereof; or a pharmaceutically acceptable salt, solvate or prodrug thereof.

144. The compound of claim 133, wherein the trigger element and the immolative element together comprise one of the following structures: 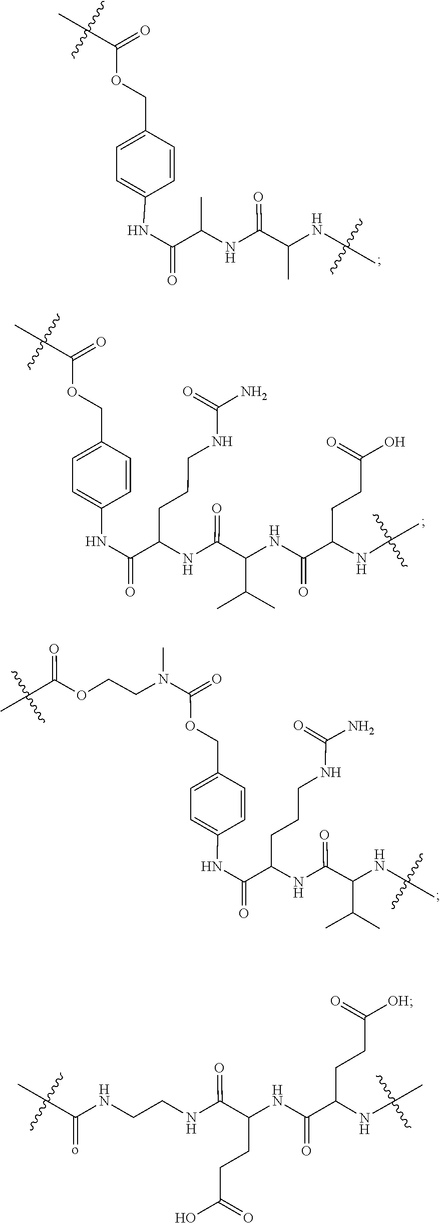 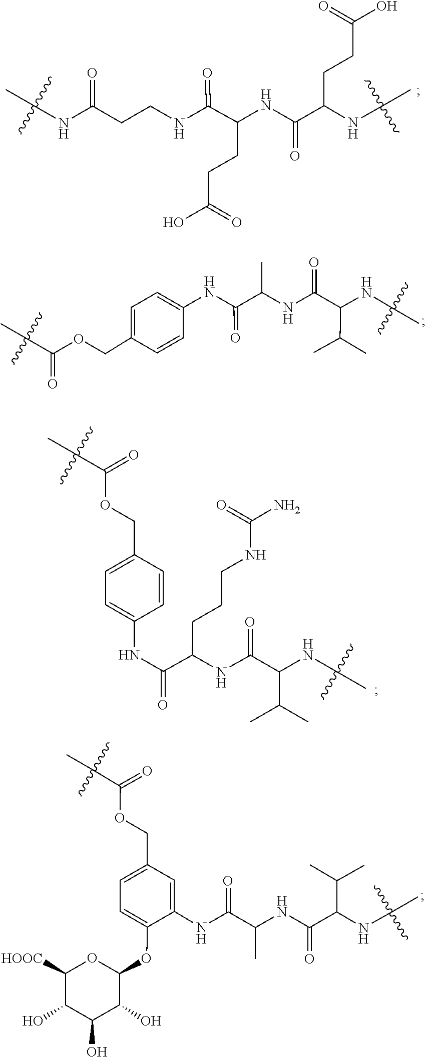 [SMILES: *NC(=O)CCNC(=O)C(CCC(=O)O)NC(=O)C(CCC(=O)O)N* ; *NC(C(=O)NC(C)C(=O)Nc1ccc(COC(*)=O)cc1)C(C)C ; *NC(C(=O)NC(CCCNC(N)=O)C(=O)Nc1ccc(COC(*)=O)cc1)C(C)C ; *NC(C(=O)NC(C)C(=O)Nc1cc(COC(*)=O)ccc1O[C@@H]1O[C@H](C(=O)O)[C@@H](O)[C@H](O)[C@H]1O)C(C)C] 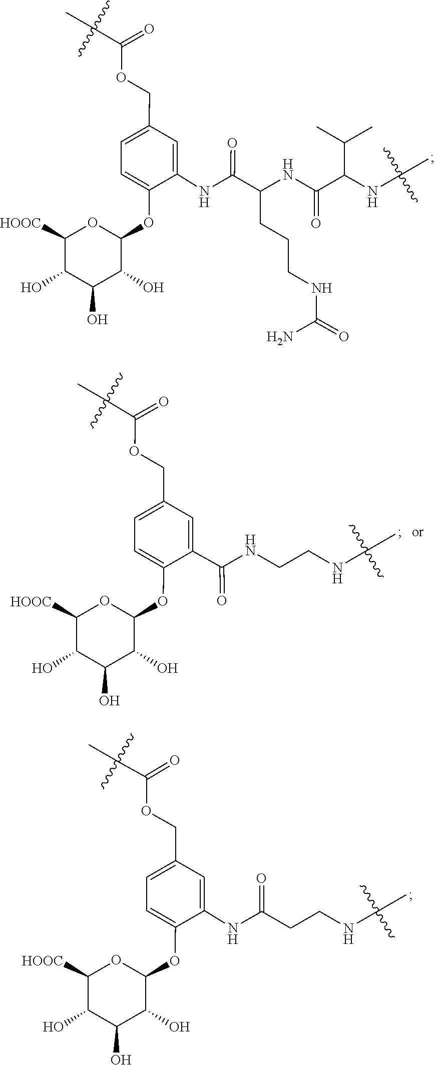 [SMILES: *NC(C(=O)NC(CCCNC(N)=O)C(=O)Nc1cc(COC(*)=O)ccc1O[C@@H]1O[C@H](C(=O)O)[C@@H](O)[C@H](O)[C@H]1O)C(C)C ; *NCCNC(=O)c1cc(COC(*)=O)ccc1O[C@@H]1O[C@H](C(=O)O)[C@@H](O)[C@H](O)[C@H]1O ; *NCCC(=O)Nc1cc(COC(*)=O)ccc1O[C@@H]1O[C@H](C(=O)O)[C@@H](O)[C@H](O)[C@H]1O]
as a stereoisomer, enantiomer, or tautomer thereof or a mixture thereof; or a pharmaceutically acceptable salt, solvate or prodrug thereof.

145. The compound of claim 133, wherein the polar cap is —NH₂, —OH or —C(═O)—OH, as a stereoisomer, enantiomer, or tautomer thereof or a mixture thereof; or a pharmaceutically acceptable salt, solvate or prodrug thereof.

146. The compound of claim 133, wherein the polar cap has one of the following structures, including combinations thereof: 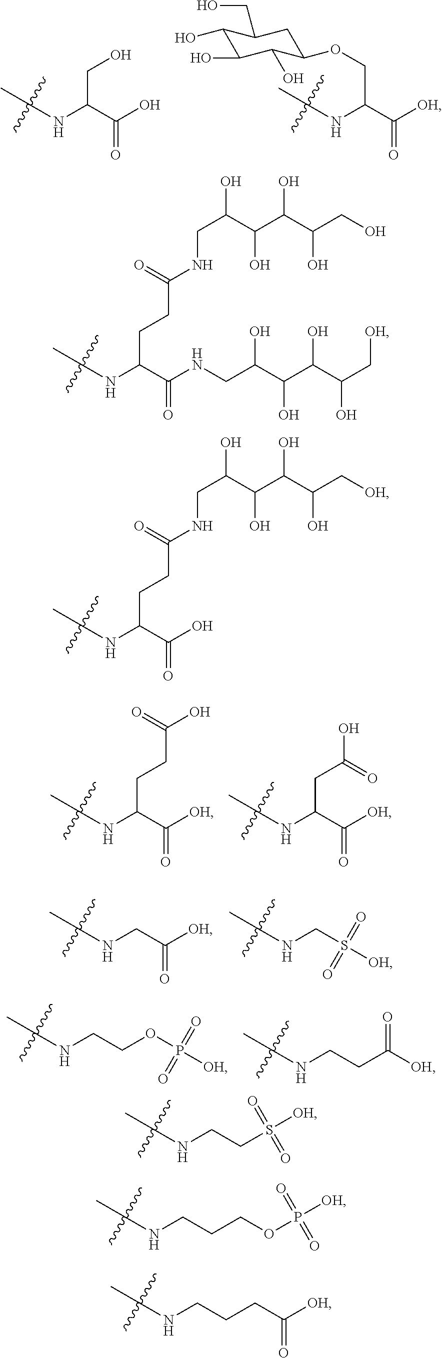 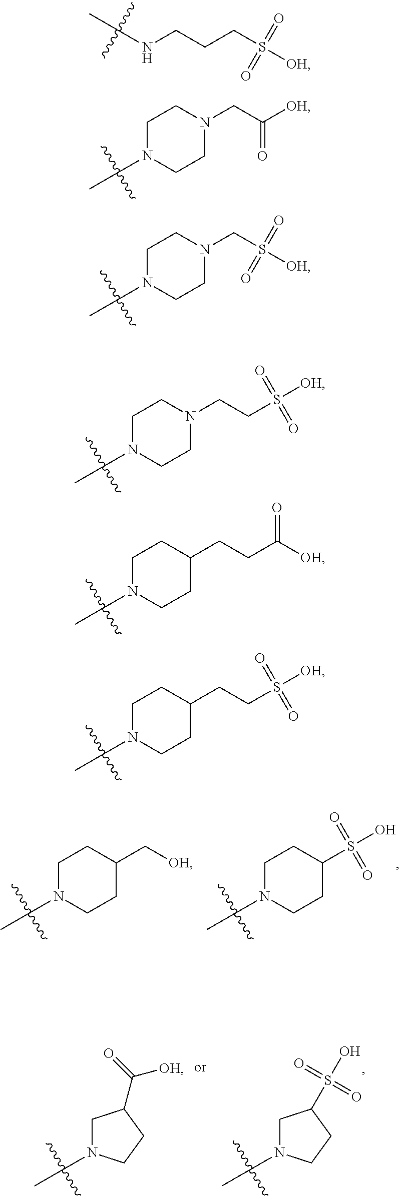
as a stereoisomer, enantiomer, or tautomer thereof or a mixture thereof; or a pharmaceutically acceptable salt, solvate or prodrug thereof.

147. The compound of claim 133, wherein the payload is selected from the group consisting of an alkylating agent, an antimetabolite, a microtubule inhibitor, a topoisomerase inhibitor, a myeloid agonist, a glucocorticoid receptor agonist, and a cytotoxic antibiotic, as a stereoisomer, enantiomer, or tautomer thereof or a mixture thereof; or a pharmaceutically acceptable salt, solvate or prodrug thereof.

148. The compound of claim 133, wherein the cytotoxic agent selected from the group consisting of calicheamicin, anthramycin, abbeymycin, chicamycin, DC-81, mazethramycin, neothramycin A, neothramycin B, porothramycin prothracarcin, sibanomicin, sibiromycin, tomamycin, auristatin F, monomethyl auristatin F, auristatin E, monomethyl auristatin E, dolastatin, monomethyl dolastatin, mertansine, and emtansine, as a stereoisomer, enantiomer, or tautomer thereof or a mixture thereof; or a pharmaceutically acceptable salt, solvate or prodrug thereof.

149. The compound of claim 133, wherein the myeloid cell agonist selected from the group consisting of a STING agonist, a ligand of TLR2, a ligand of TLR3, a ligand of TLR4, a ligand of TLR5, a ligand of TLR6, a ligand of TLR7, a ligand of TLR8, a ligand of TLR9, a ligand of TLR10, a ligand of nucleotide-oligomerization domain (NOD), a ligand of an RIG-I-Like Receptors (RLR), a ligand of a C-type lectin receptor (CLR), a ligand of a Cytosolic DNA Sensor (CDS) and a ligand of an inflammasome inducer, as a stereoisomer, enantiomer or tautomer thereof or a mixture thereof; or a pharmaceutically acceptable salt, solvate or prodrug thereof.

150. The compound of claim 133, wherein payload has one of the following structures: 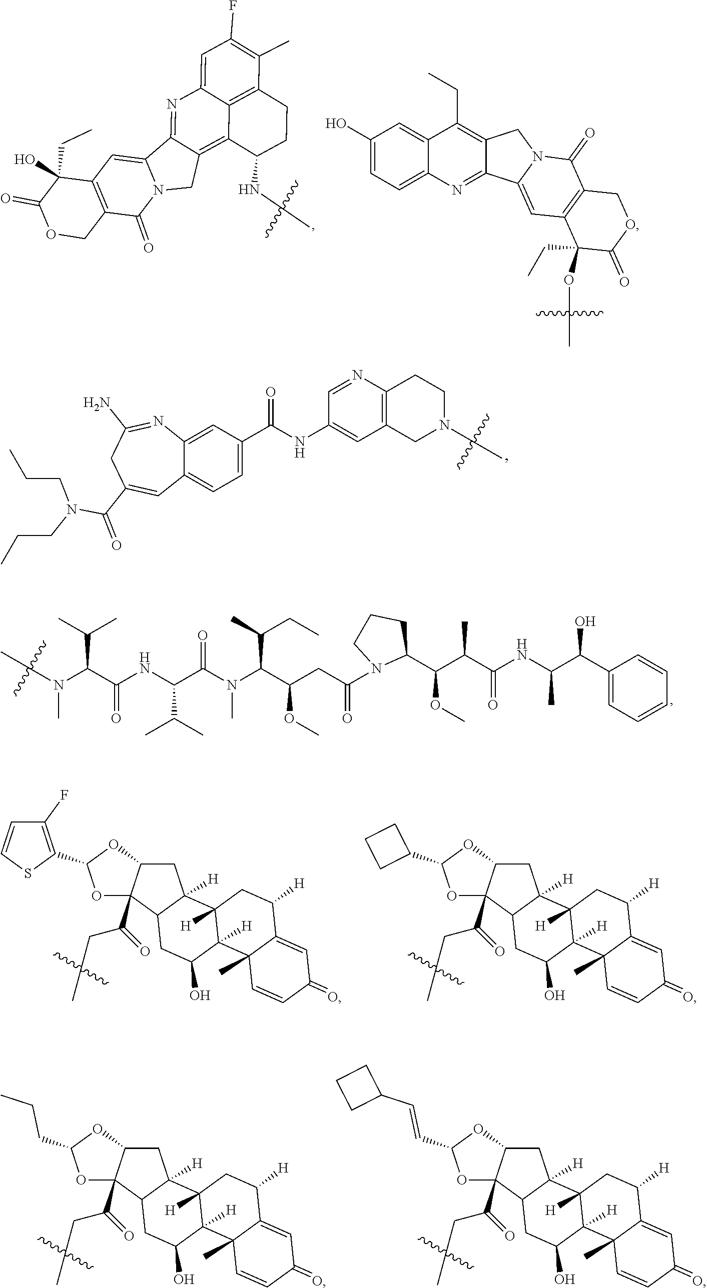 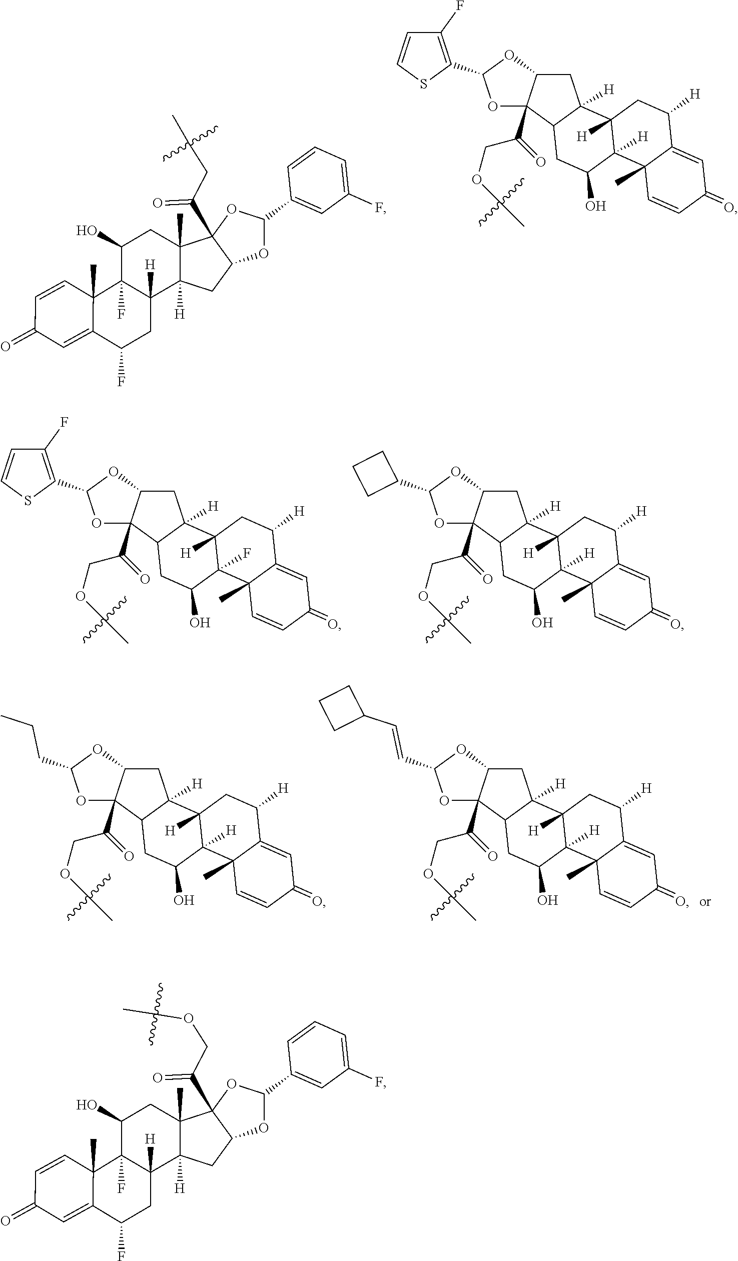
as a stereoisomer, enantiomer, or tautomer thereof or a mixture thereof; or a pharmaceutically acceptable salt, solvate or prodrug thereof.

151. The compound of claim 133, wherein the payload has one of the following structures: 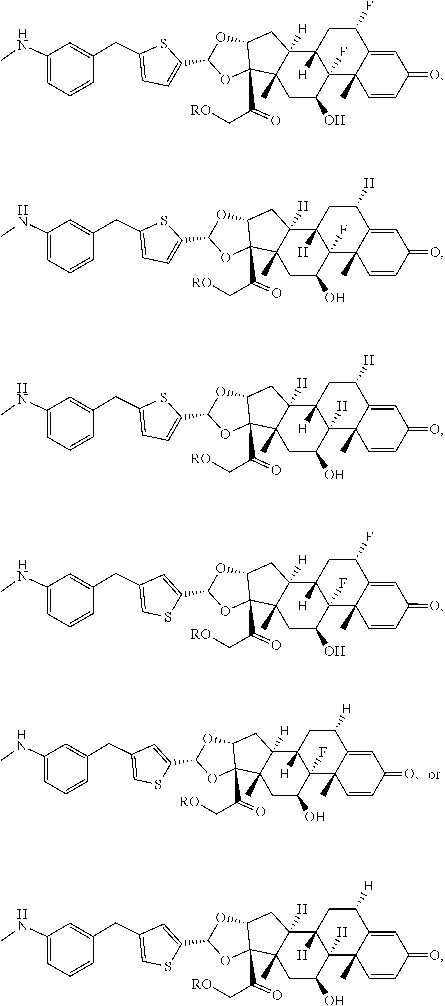
wherein:
R′ is hydrogen or has one of the following structures: 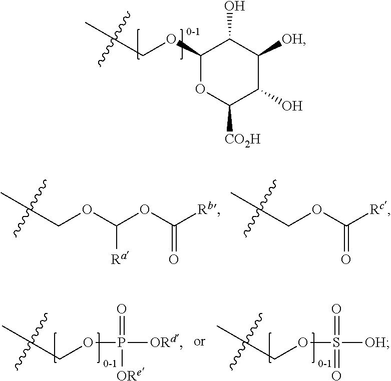
wherein:
R^{a}′ is H or C₁₋₆ alkyl;
R^{b}′ is C₁₋₆ alkyl or C₁₋₆ alkoxy;
R^{c}′ is H, C₁₋₆ alkyl, —CH₂OH, or C₁₋₆ alkoxy;
R^{d}′ is H or C₁₋₆ alkyl; or
R^{e}′ is H or C₁₋₆ alkyl, as a stereoisomer, enantiomer, or tautomer thereof or a mixture thereof; or a pharmaceutically acceptable salt, solvate or prodrug thereof.

152. The compound of claim 133, wherein the compound has one of the following structures (Ia-1), (Ia-2), (Ia-3), (Ia-4), (Ia-5), (Ia-6), (Ia-7), or (Ia-8): 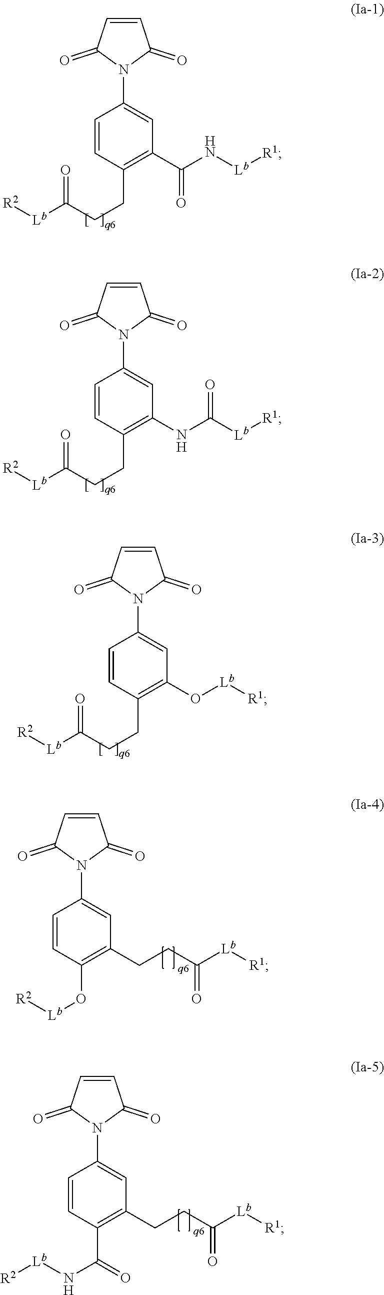 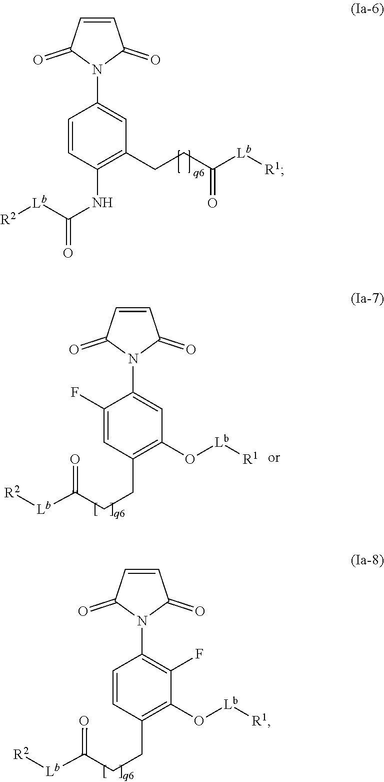
wherein:
each occurrence of L^{b} is independently a direct bond, an optionally substituted alkylene linker, an optionally substituted heteroalkylene linker, a heteroatomic linker, or combinations thereof; and
q6 is 0, 1, or 2,
as a stereoisomer, enantiomer, or tautomer thereof or a mixture thereof; or a pharmaceutically acceptable salt, solvate or prodrug thereof.

153. The compound of claim 133, wherein the compound has one of the following structures: 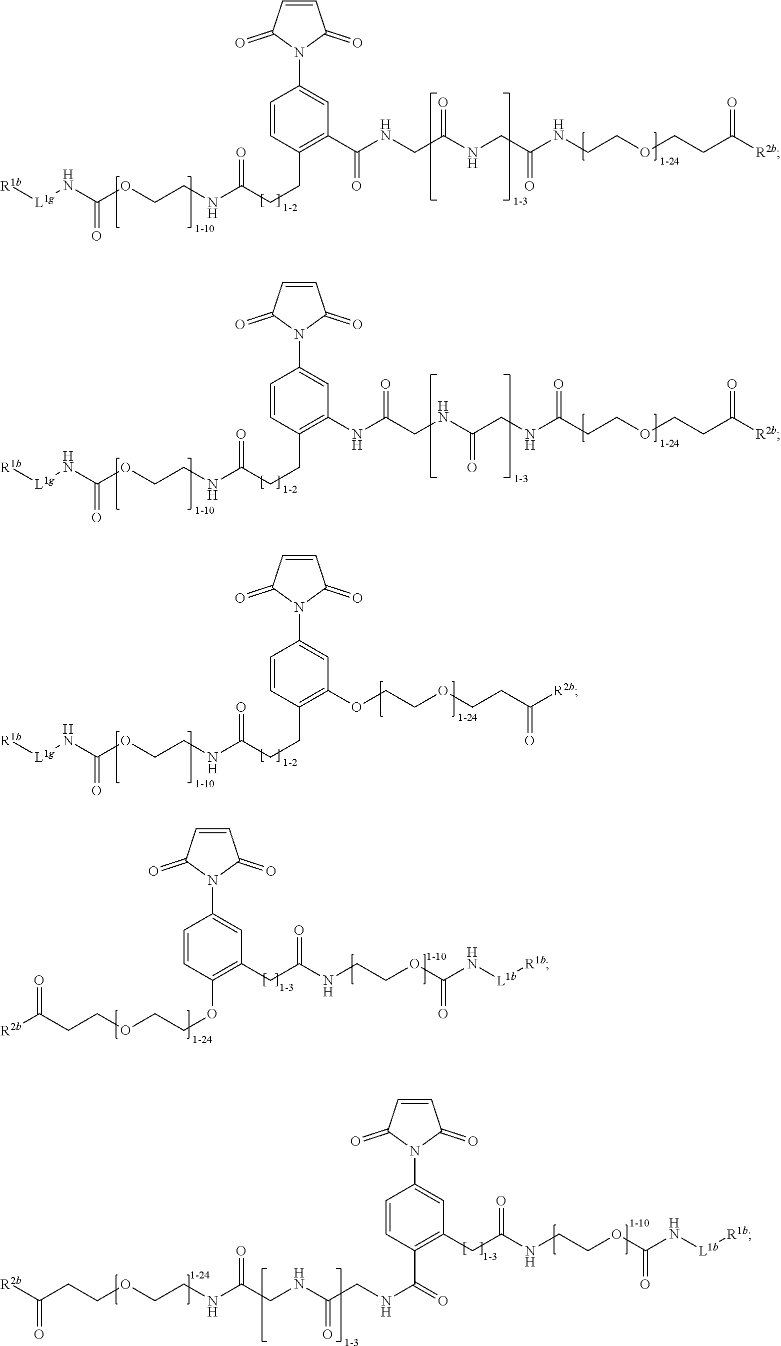 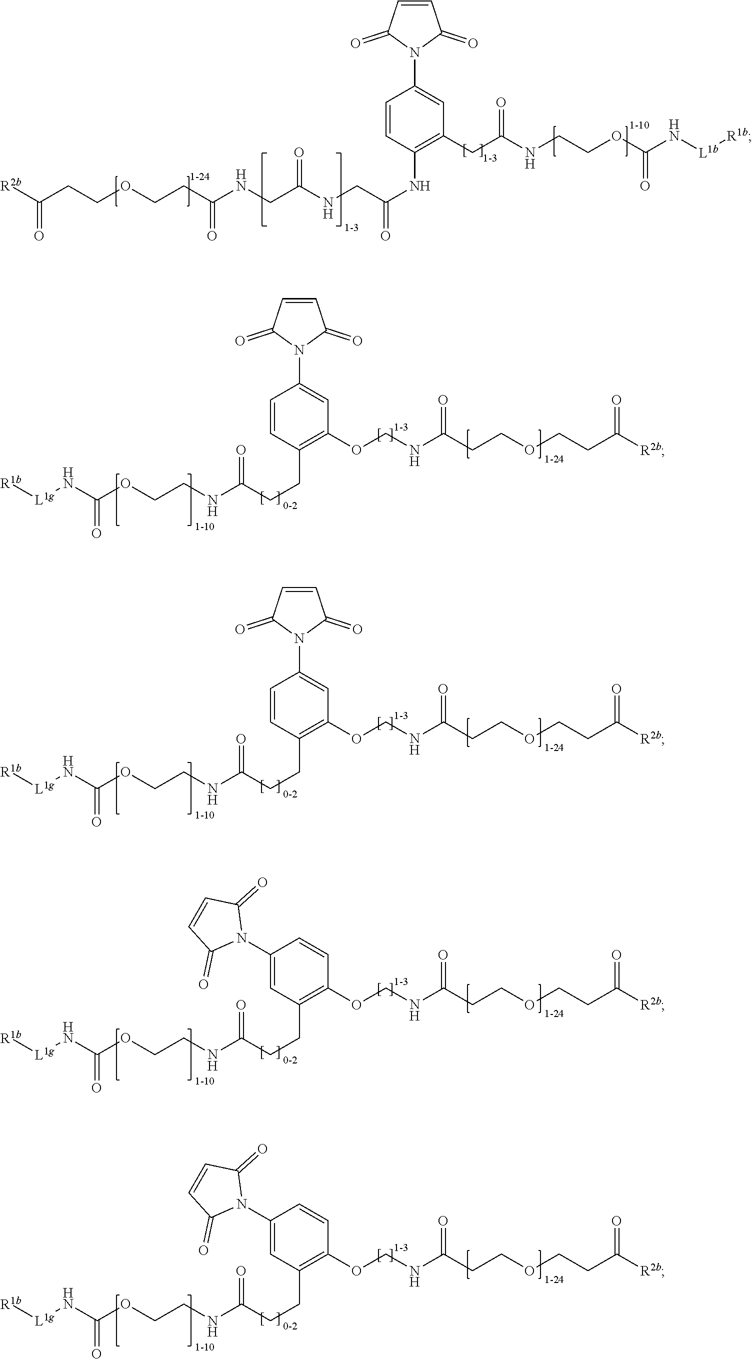 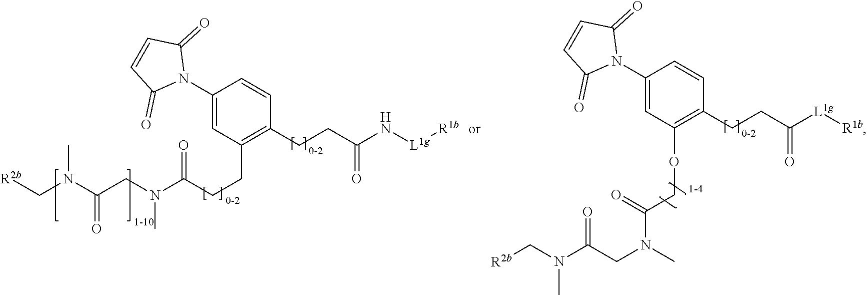
wherein:
R^{1b} is the payload;
R^{2b} has one of the following structures: 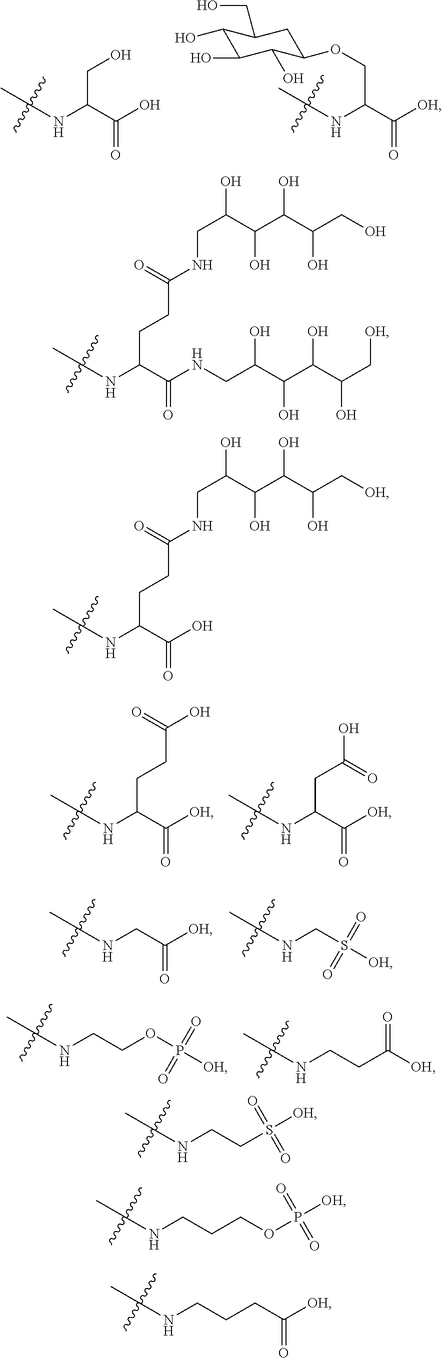 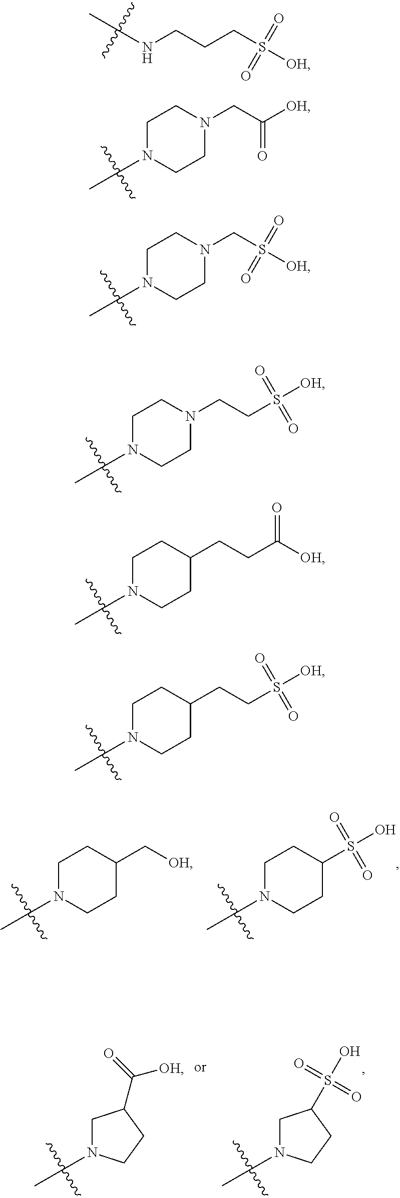
L^{1g} has one of the following structures: 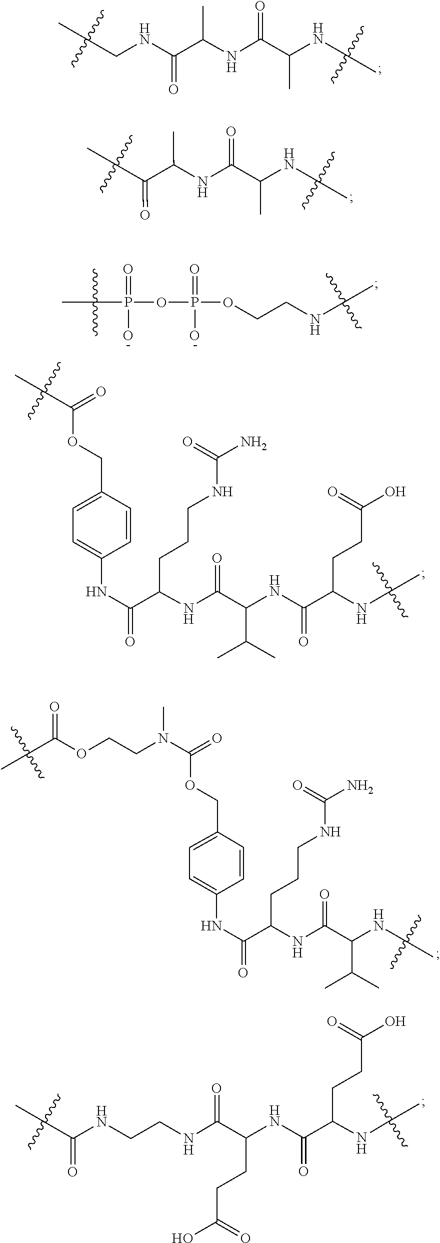 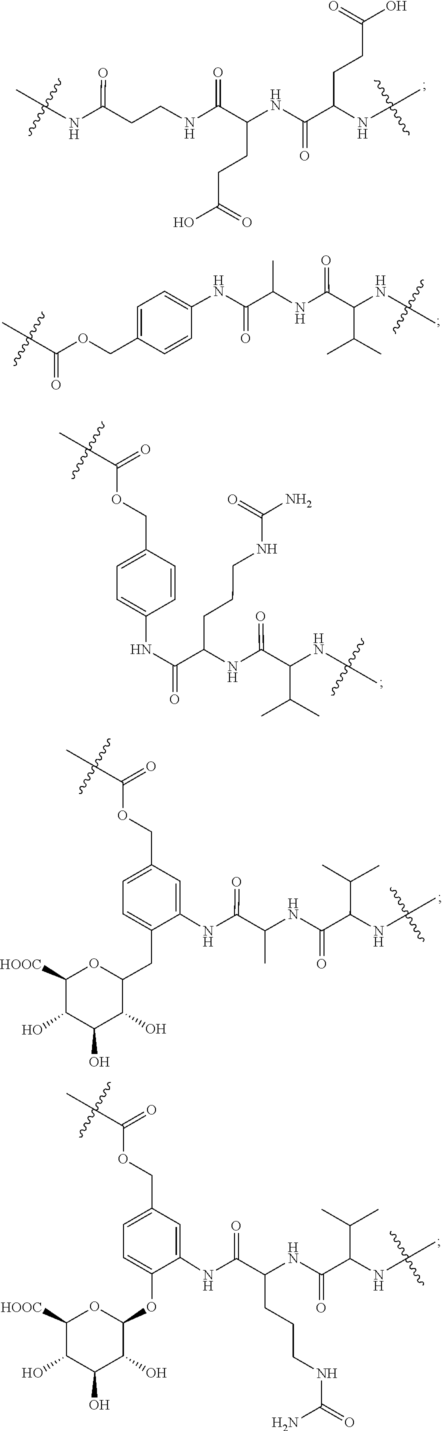 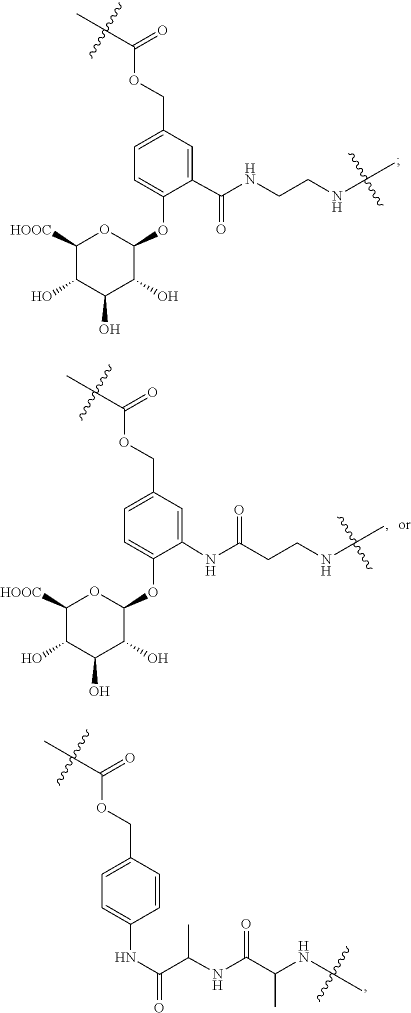 [SMILES: *NCCNC(=O)c1cc(COC(*)=O)ccc1O[C@@H]1O[C@H](C(=O)O)[C@@H](O)[C@H](O)[C@H]1O ; *NCCC(=O)Nc1cc(COC(*)=O)ccc1O[C@@H]1O[C@H](C(=O)O)[C@@H](O)[C@H](O)[C@H]1O ; *NC(C)C(=O)NC(C)C(=O)Nc1ccc(COC(*)=O)cc1]
as a stereoisomer, enantiomer, or tautomer thereof or a mixture thereof; or a pharmaceutically acceptable salt, solvate or prodrug thereof.

154. The compound of claim 133, wherein the compound has one of the following structures: 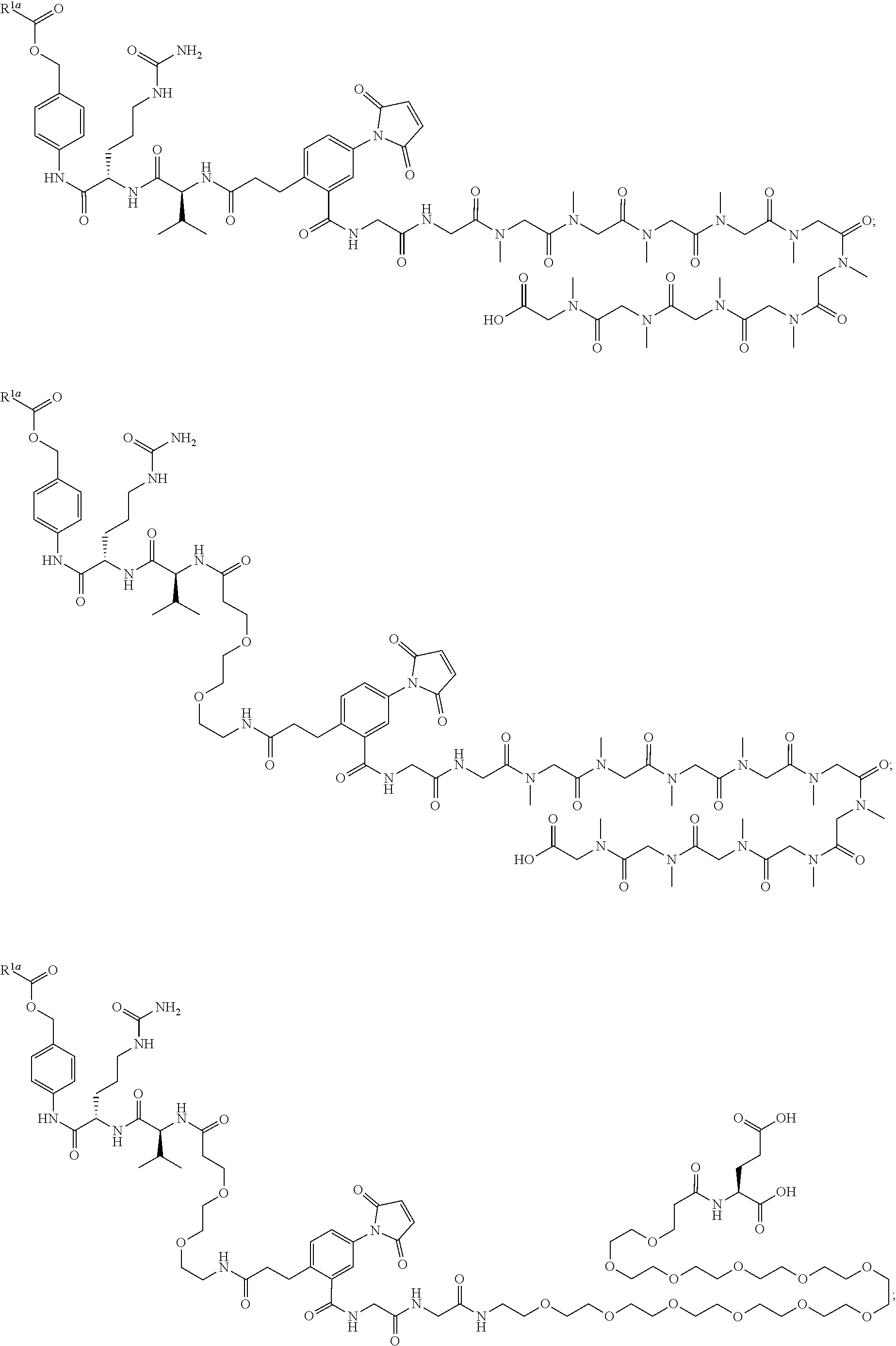 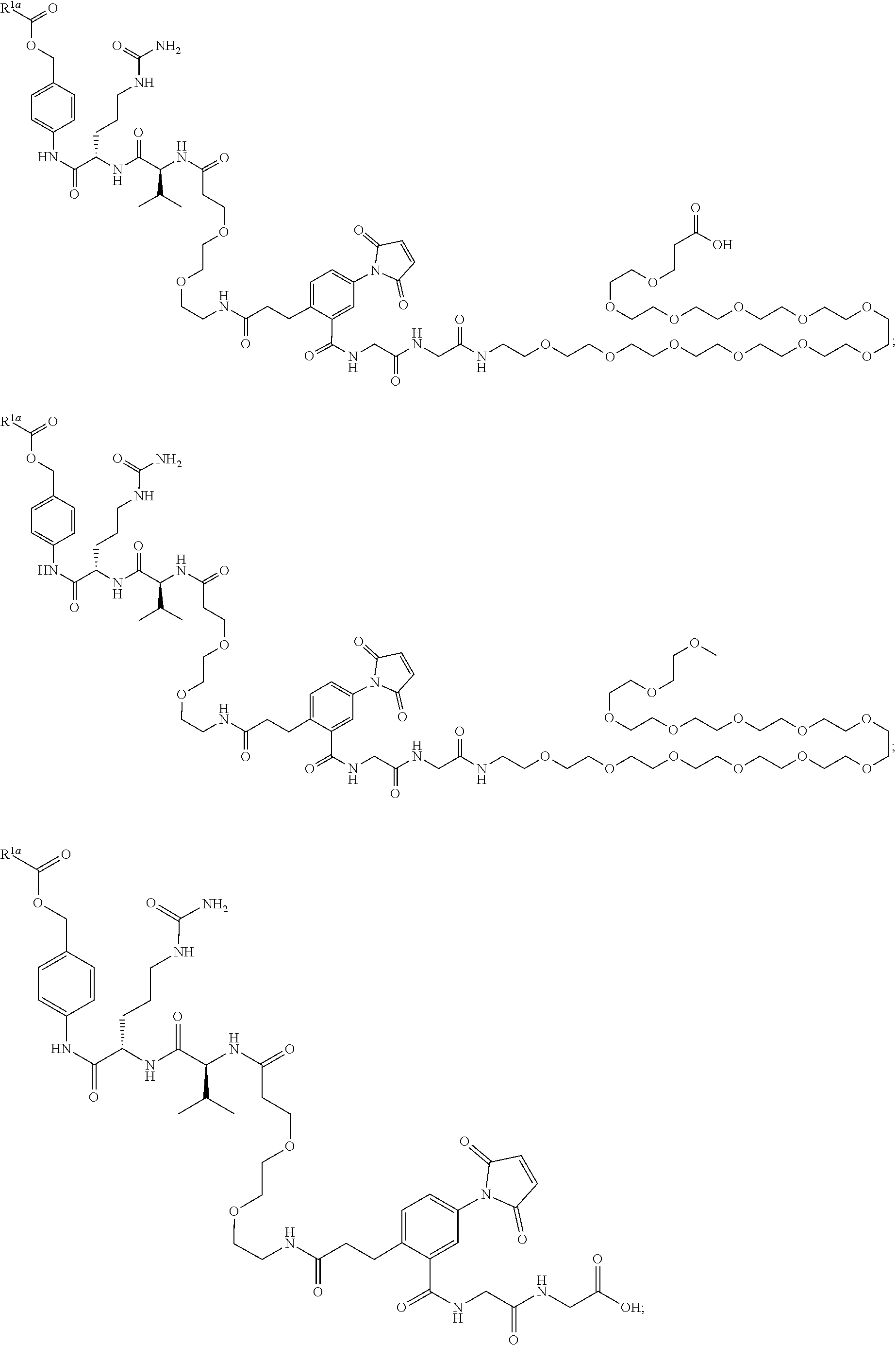 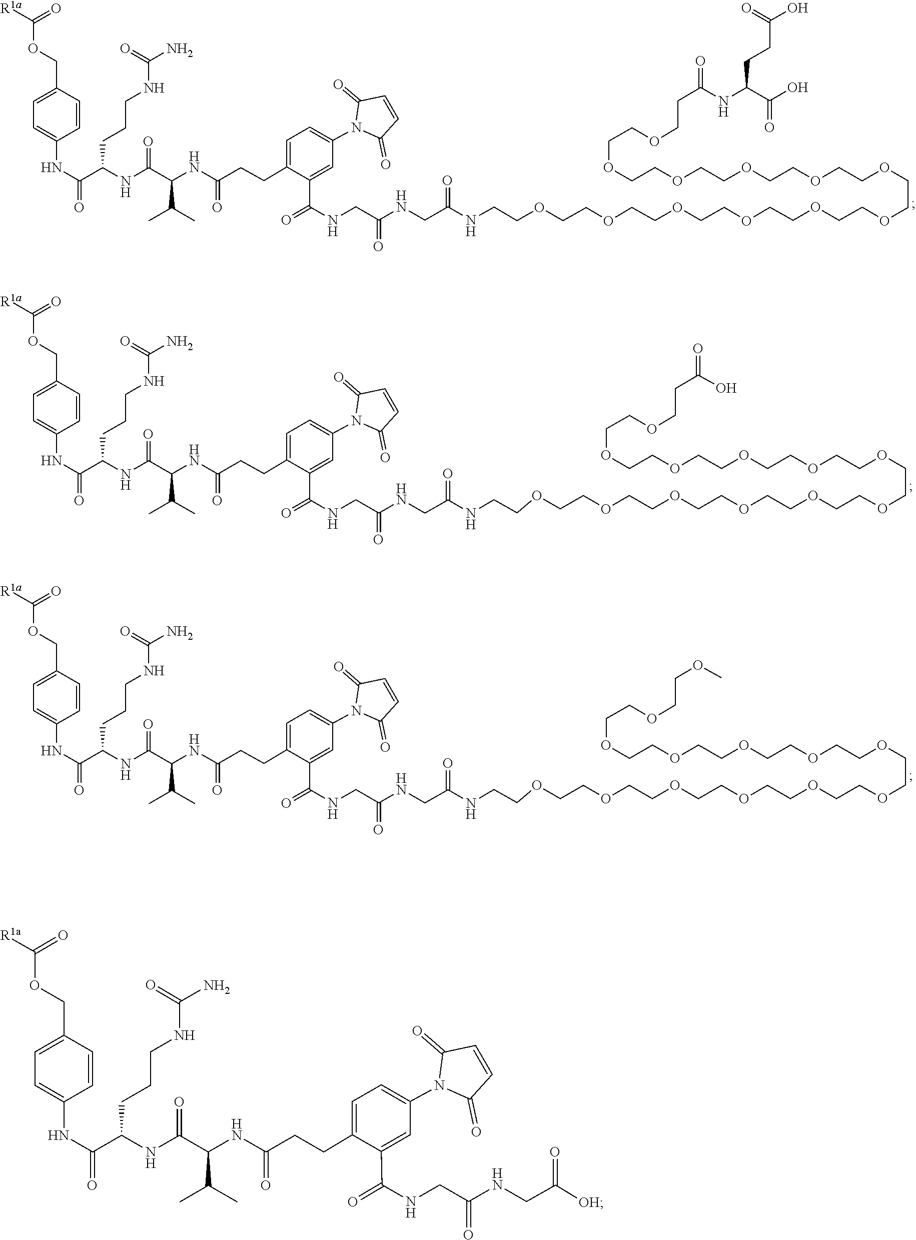 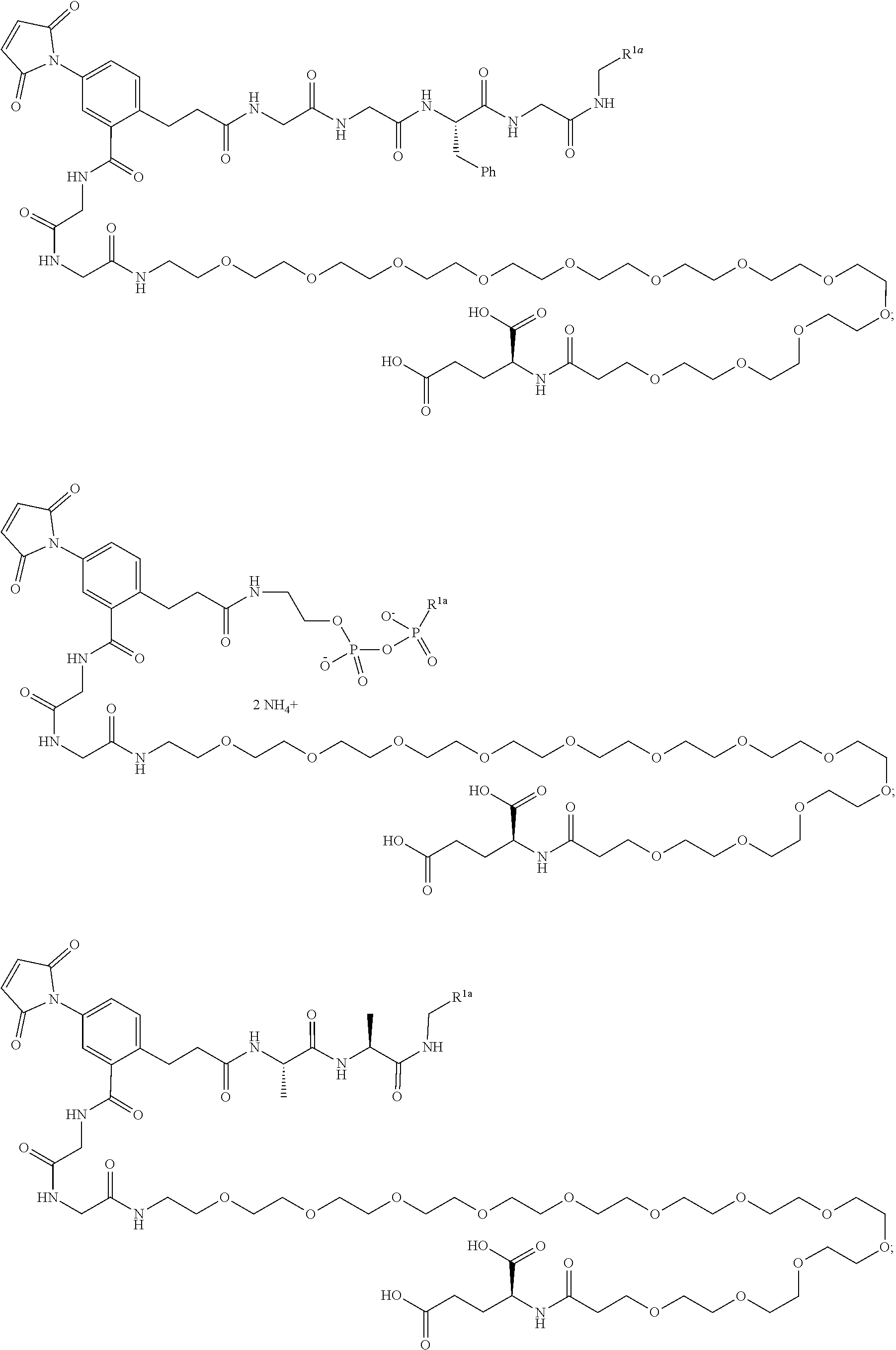 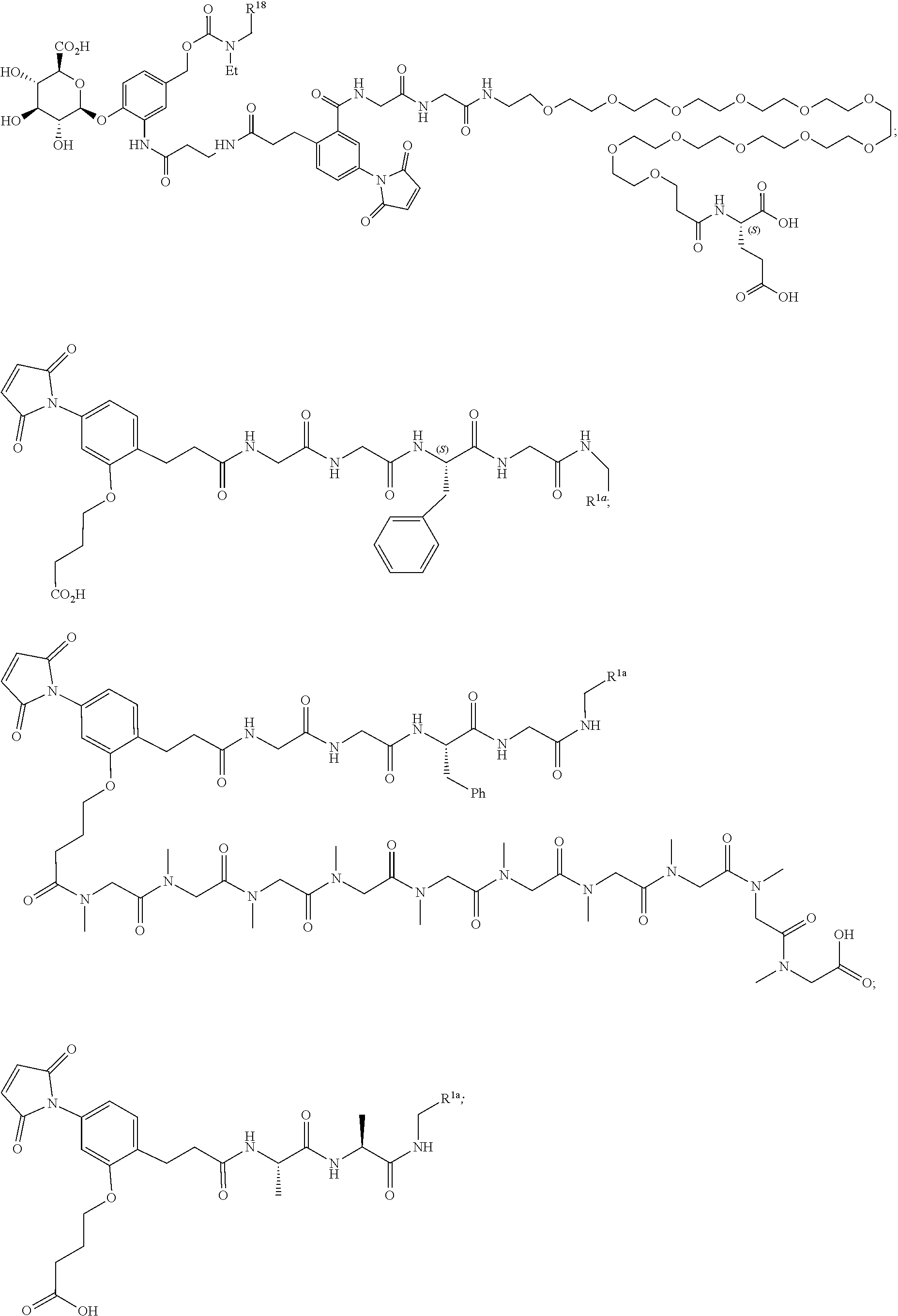 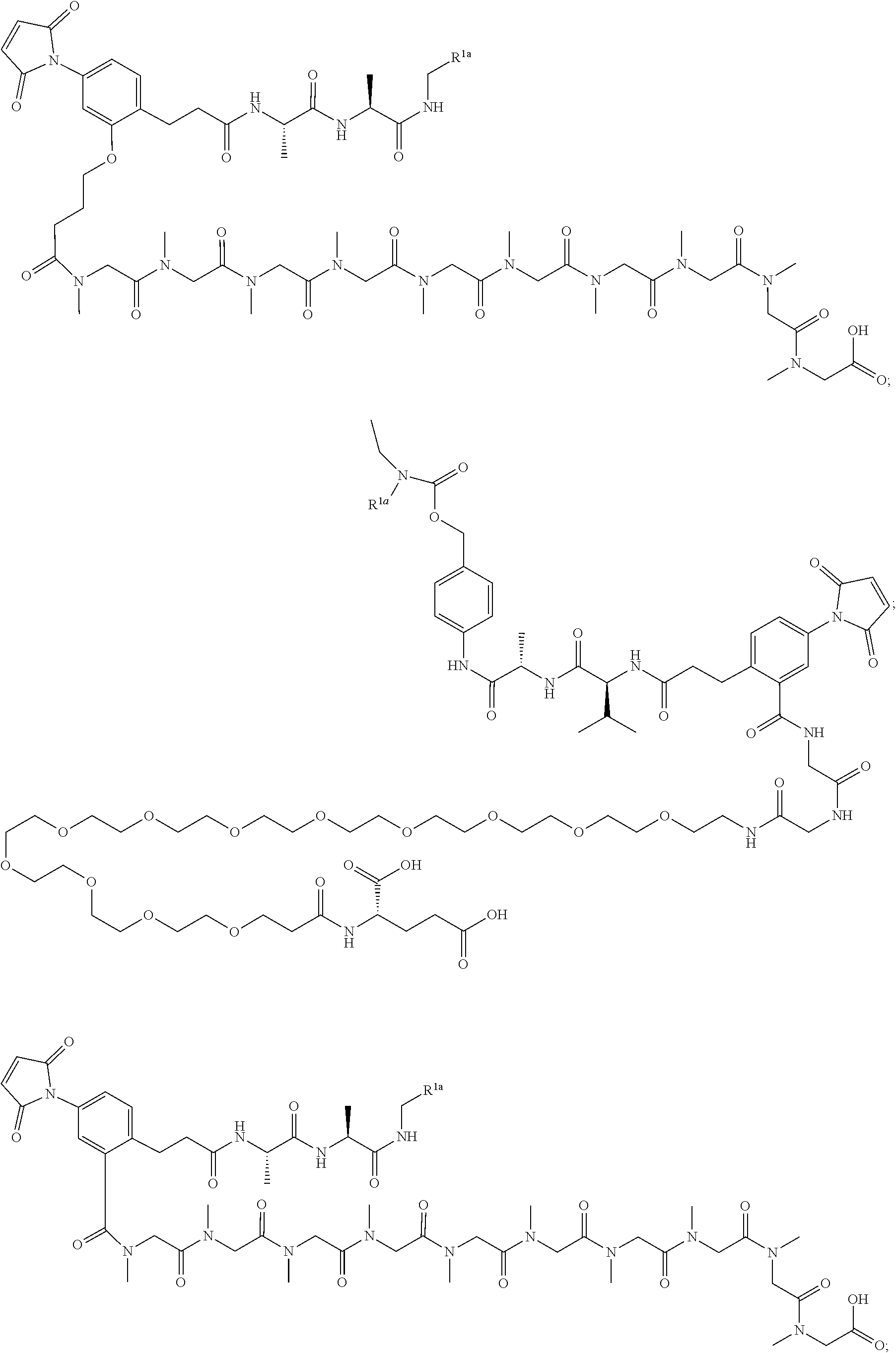 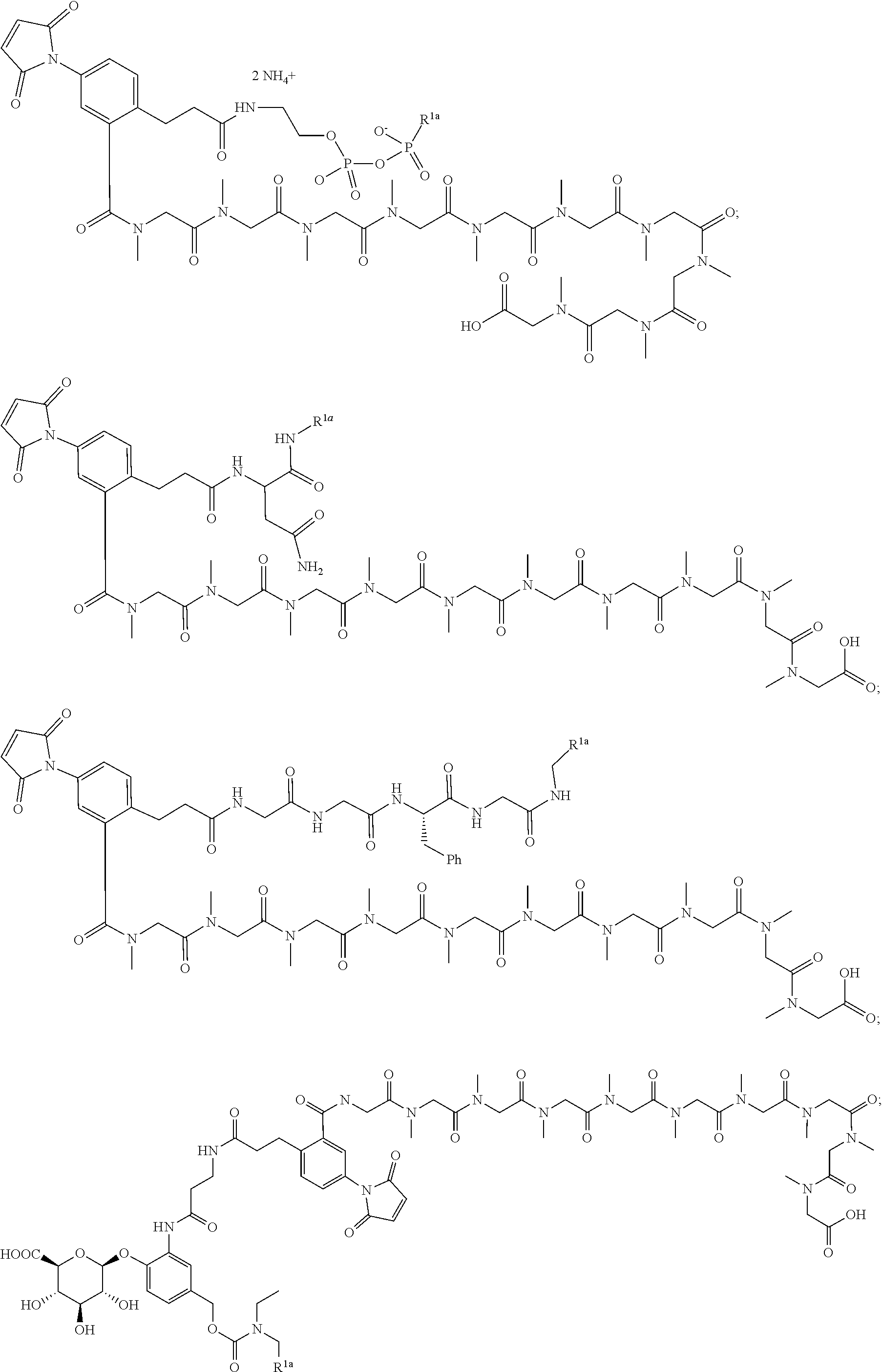 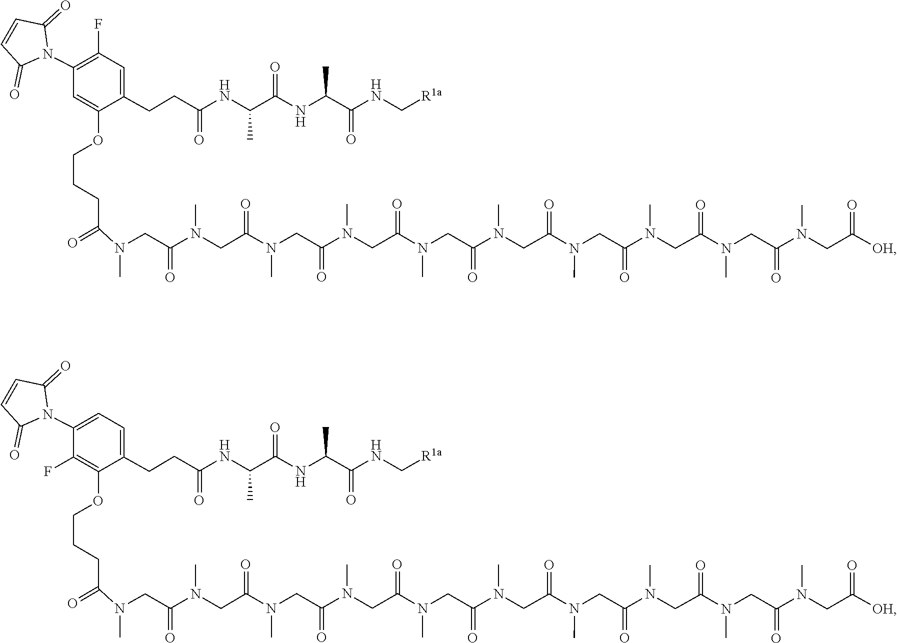
as a stereoisomer, enantiomer, or tautomer thereof or a mixture thereof; or a pharmaceutically acceptable salt, solvate or prodrug thereof.

155. The compound of claim 154, wherein R^{1a} has one of the following structures: 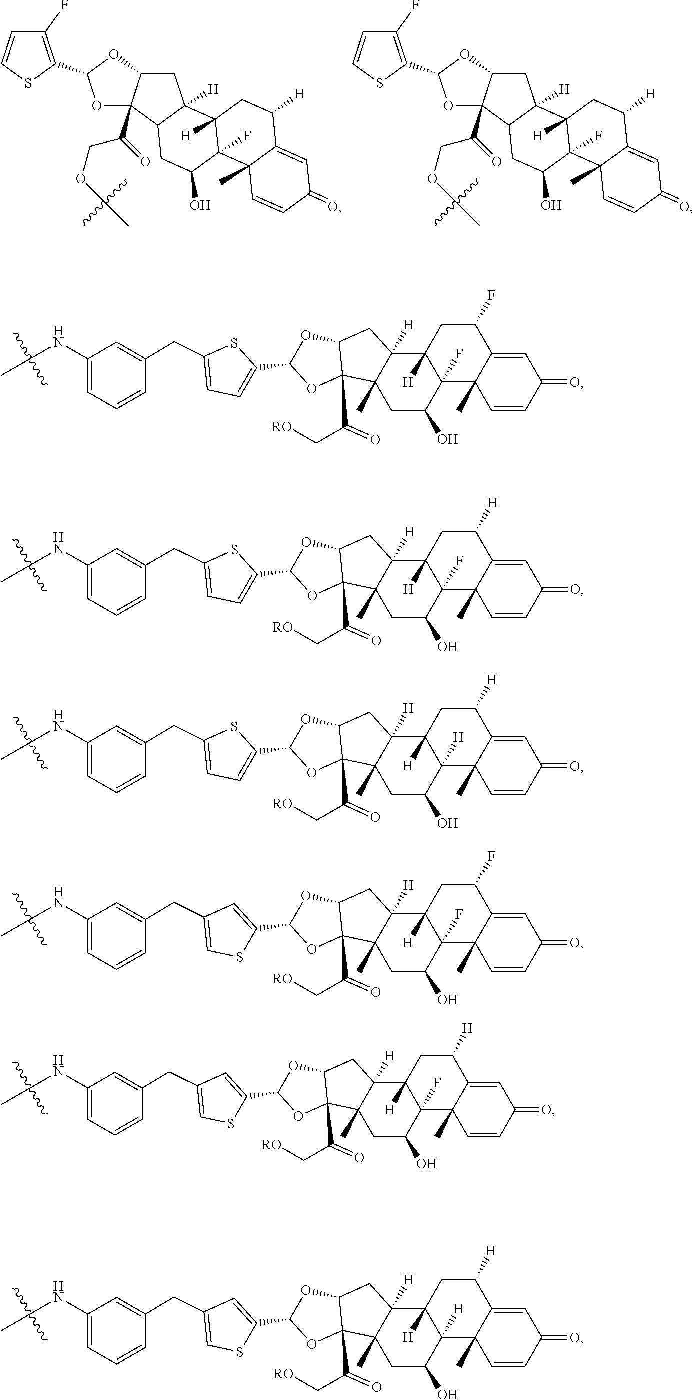 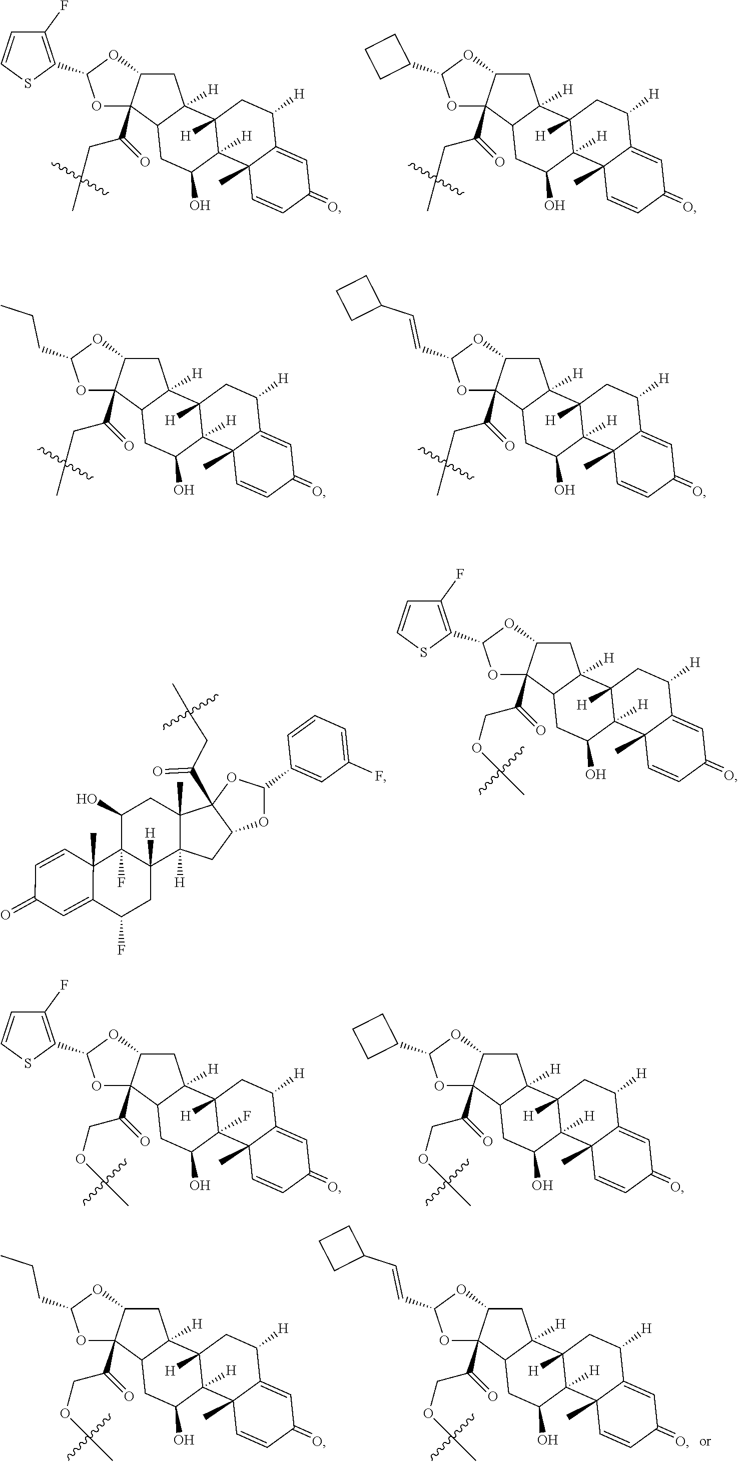 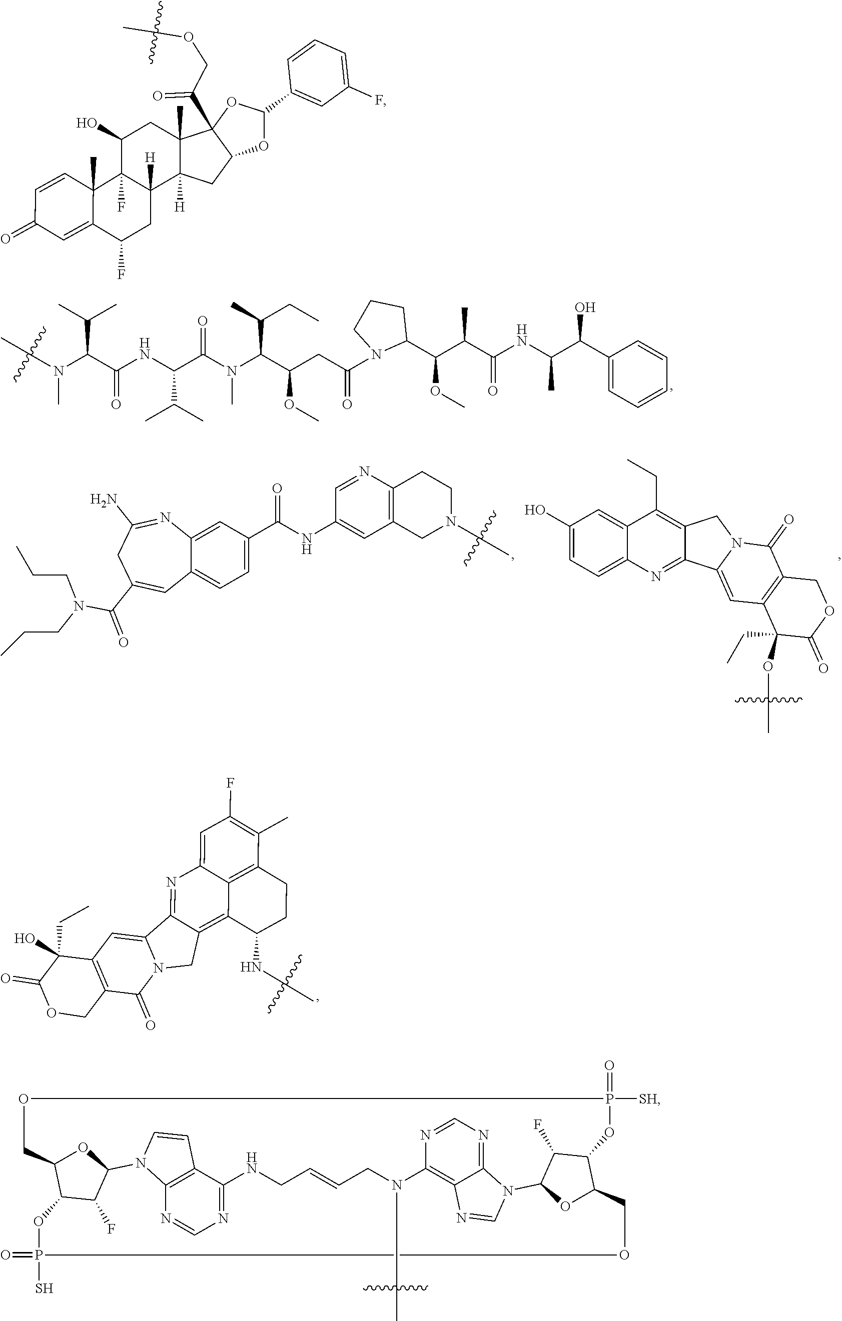 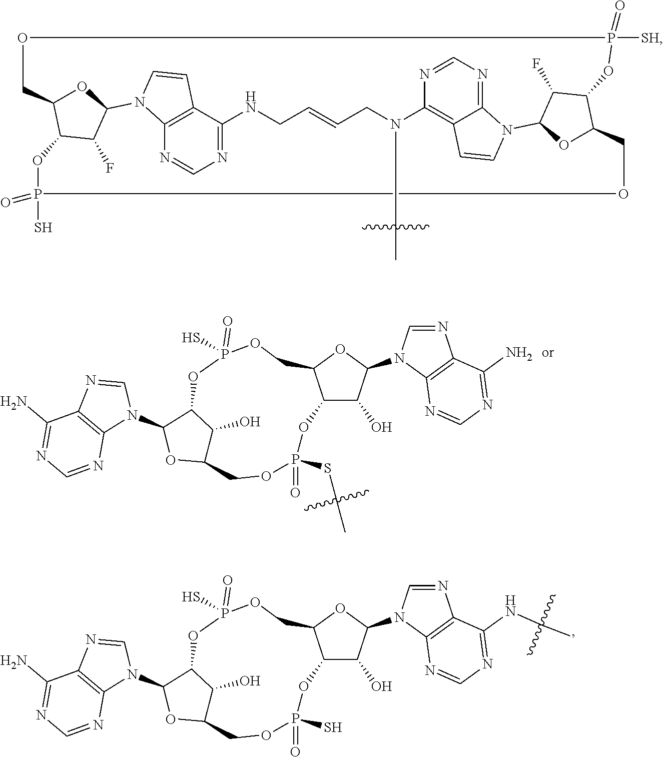 [SMILES: *N1C/C=C/CNc2ncnc3c2ccn3[C@@H]2O[C@@H]3COP(=O)(S)O[C@H]4[C@@H](F)[C@@H](O[C@@H]4COP(=O)(S)O[C@H]3[C@H]2F)n2ccc3c1ncnc32 ; *S[P@@]1(=O)OC[C@H]2O[C@@H](n3cnc4c(N)ncnc43)[C@H](O[P@](=O)(S)OC[C@H]3O[C@@H](n4cnc5c(N)ncnc54)[C@H](O)[C@@H]3O1)[C@@H]2O ; *Nc1ncnc2c1ncn2[C@@H]1O[C@@H]2CO[P@@](=O)(S)O[C@@H]3[C@H](O)[C@@H](CO[P@](=O)(S)O[C@H]2[C@H]1O)O[C@H]3n1cnc2c(N)ncnc21]
as a stereoisomer, enantiomer, or tautomer thereof or a mixture thereof; or a pharmaceutically acceptable salt, solvate or prodrug thereof.

156. The compound of claim 154, wherein R^{1a} has one of the following structures: 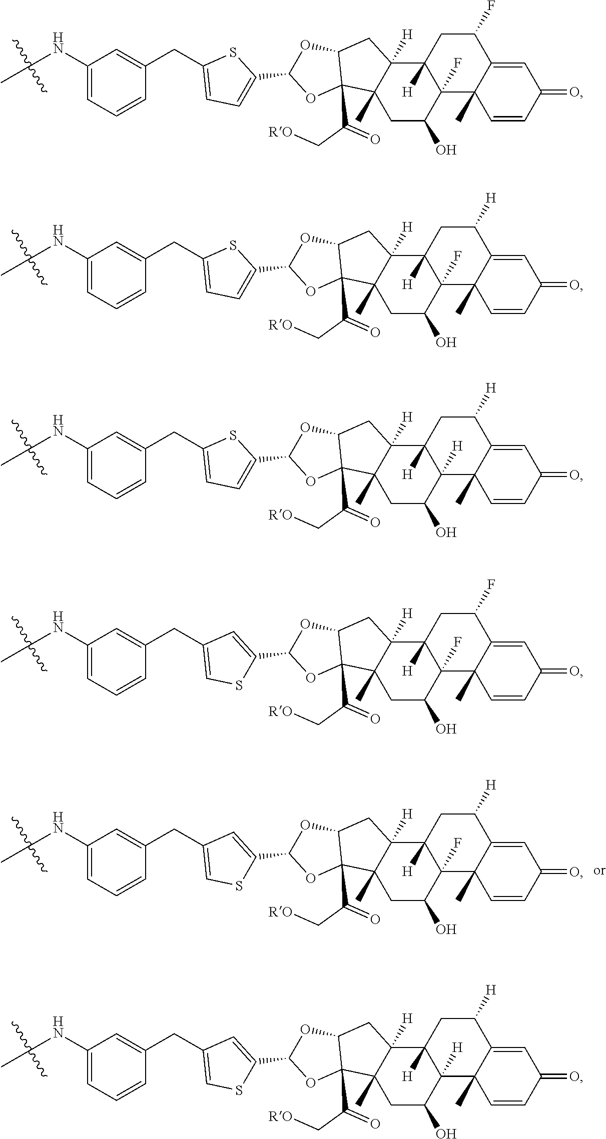
wherein:
R′ is hydrogen or has one of the following structures: 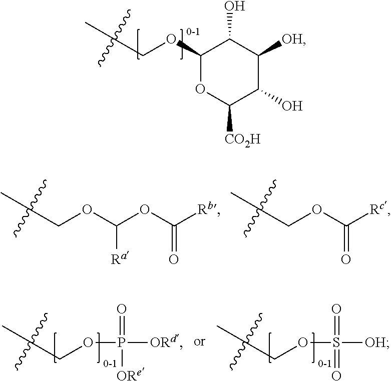
wherein:
R^{a}′ is H or C₁₋₆ alkyl;
R^{b}′ is C₁₋₆ alkyl or C₁₋₆ alkoxy;
R^{c}′ is H, C₁₋₆ alkyl, —CH₂OH, or C₁₋₆ alkoxy;
R^{d}′ is H or C₁₋₆ alkyl; or
R^{e}′ is H or C₁₋₆ alkyl as a stereoisomer, enantiomer or tautomer thereof or a mixture thereof; or a pharmaceutically acceptable salt, solvate or prodrug thereof.

157. The compound of claim 133, wherein the compound has one of the following structures: 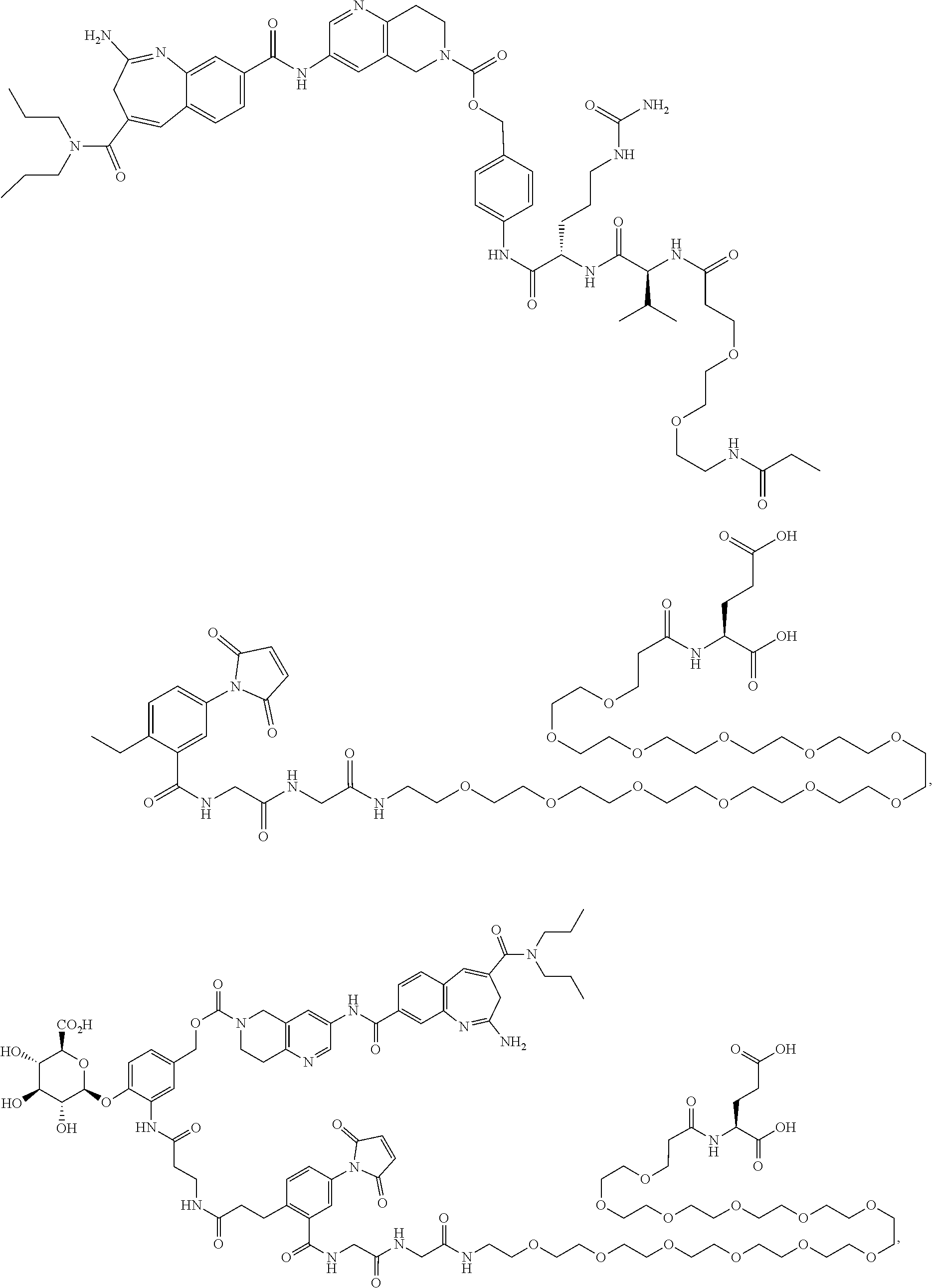 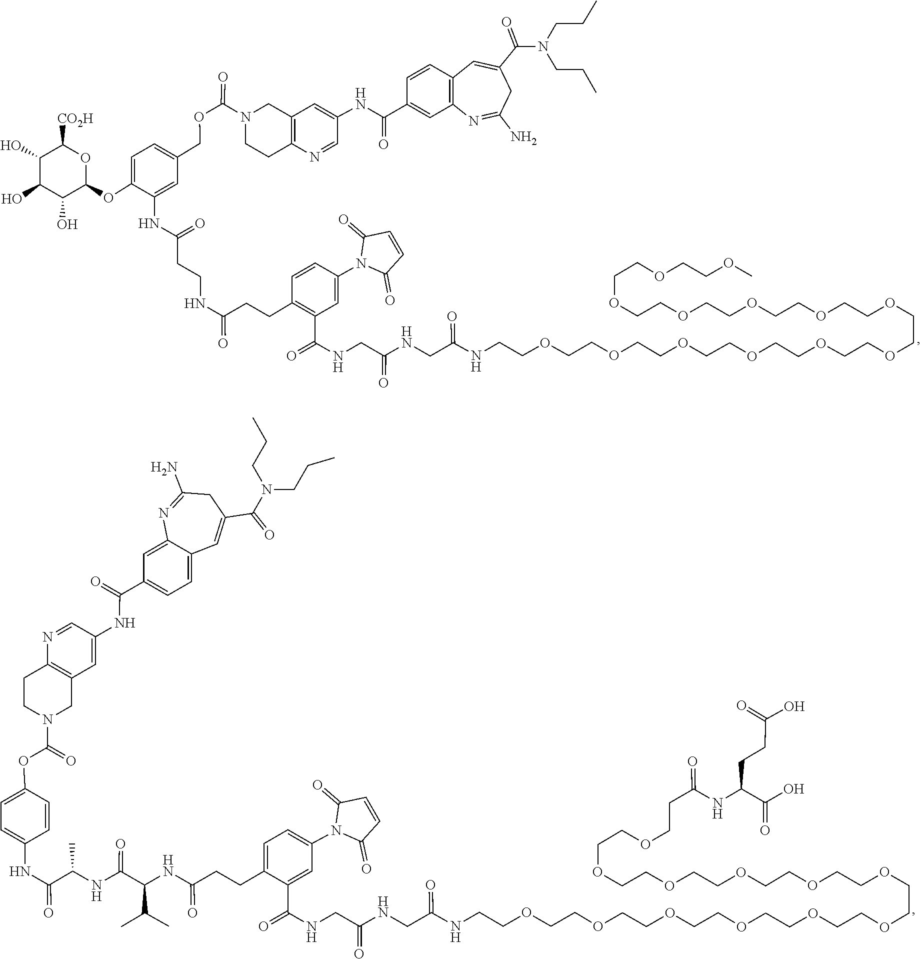 [SMILES: CCCN(CCC)C(=O)C1=Cc2ccc(C(=O)Nc3cnc4c(c3)CN(C(=O)OCc3ccc(O[C@@H]5O[C@H](C(=O)O)[C@@H](O)[C@H](O)[C@H]5O)c(NC(=O)CCNC(=O)CCc5ccc(N6C(=O)C=CC6=O)cc5C(=O)NCC(=O)NCC(=O)NCCOCCOCCOCCOCCOCCOCCOCCOCCOCCOCCOCCOCCOC)c3)CC4)cc2N=C(N)C1 ; CCCN(CCC)C(=O)C1=Cc2ccc(C(=O)Nc3cnc4c(c3)CN(C(=O)Oc3ccc(NC(=O)[C@H](C)NC(=O)[C@@H](NC(=O)CCc5ccc(N6C(=O)C=CC6=O)cc5C(=O)NCC(=O)NCC(=O)NCCOCCOCCOCCOCCOCCOCCOCCOCCOCCOCCOCCOCCC(=O)N[C@@H](CCC(=O)O)C(=O)O)C(C)C)cc3)CC4)cc2N=C(N)C1] 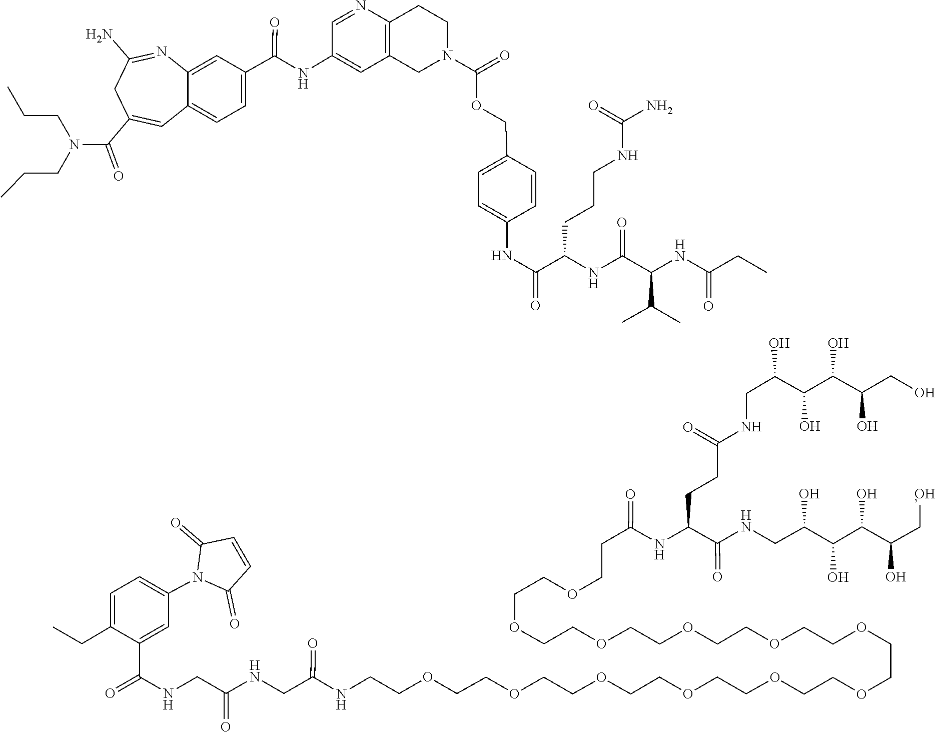 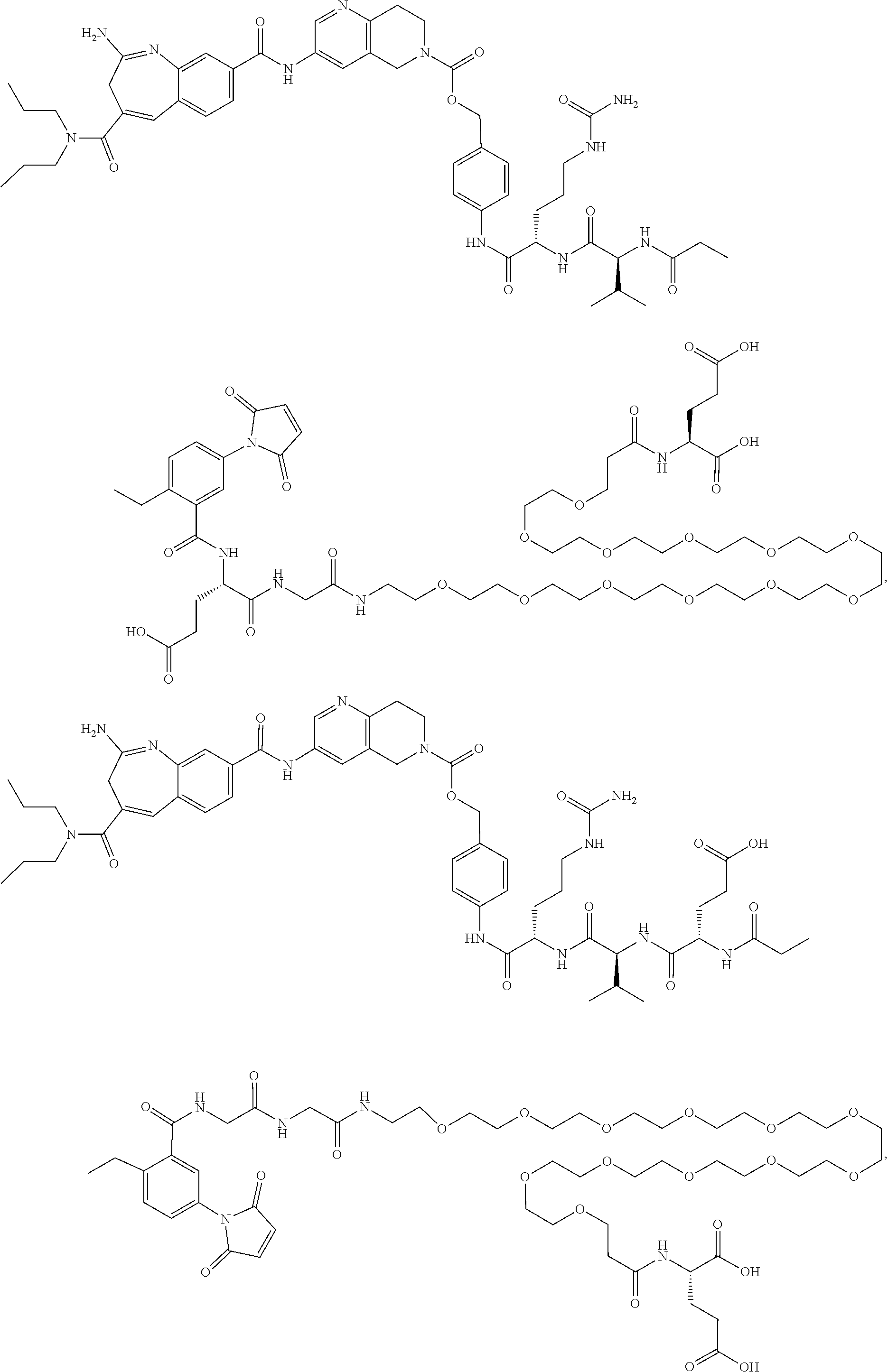 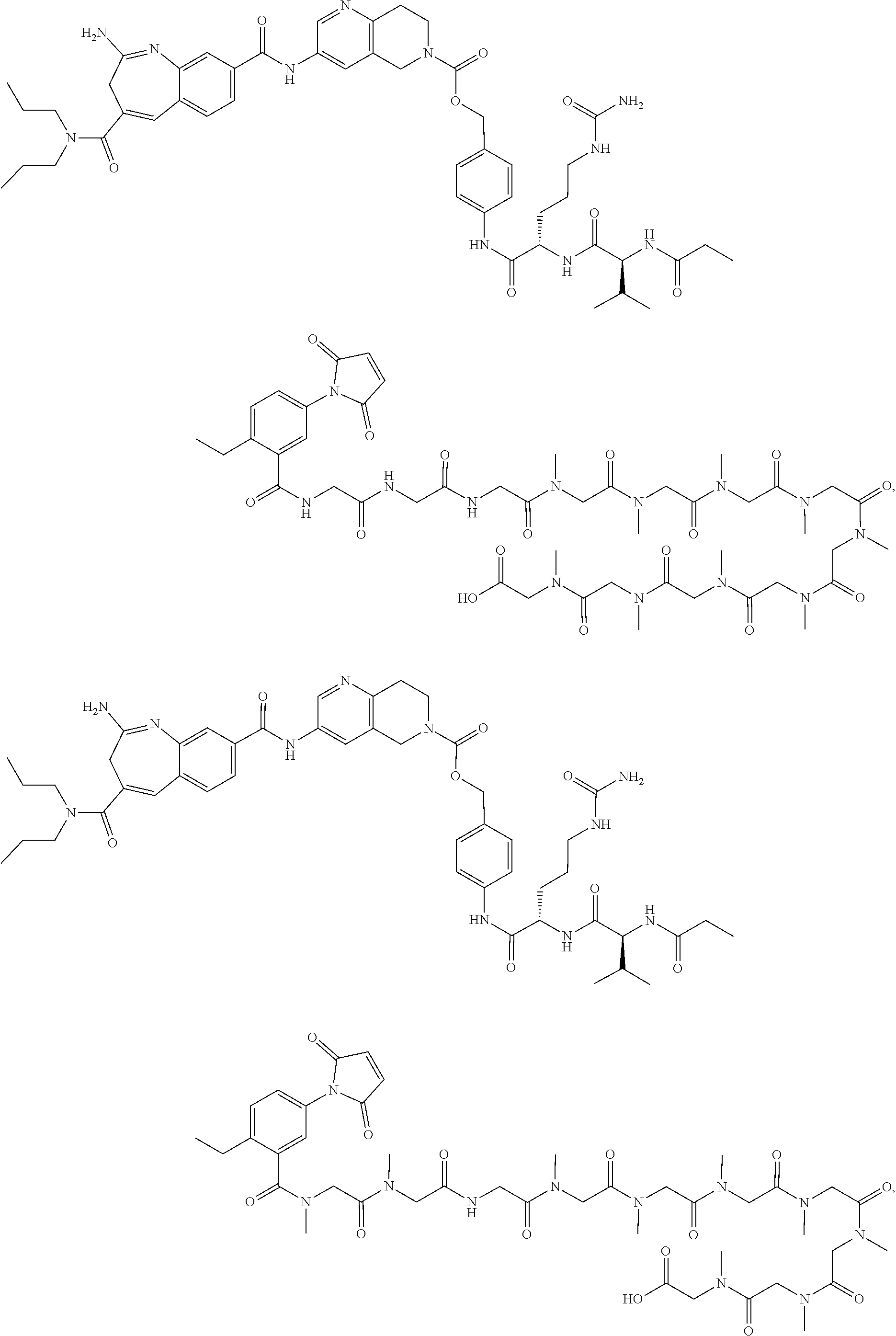 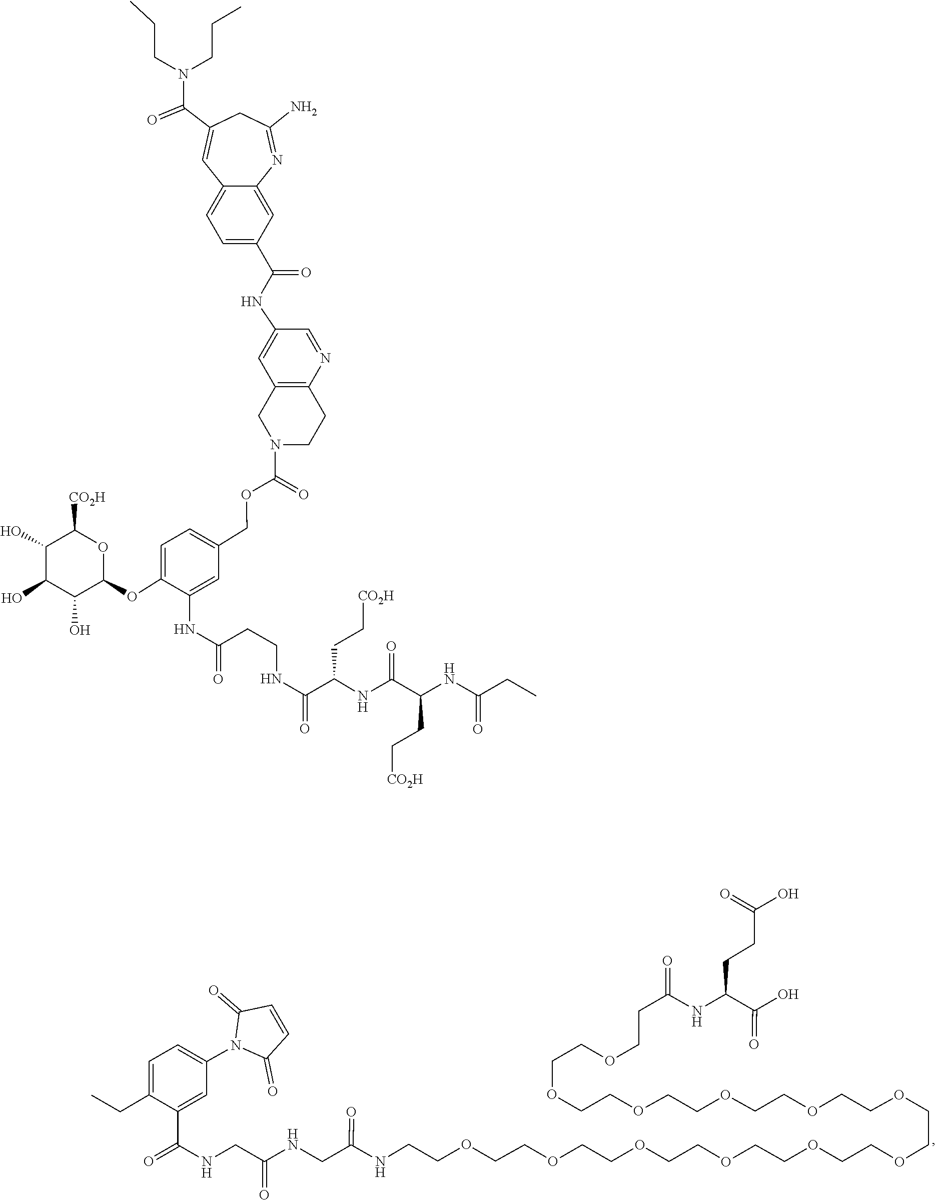 [SMILES: CCCN(CCC)C(=O)C1=Cc2ccc(C(=O)Nc3cnc4c(c3)CN(C(=O)OCc3ccc(O[C@@H]5O[C@H](C(=O)O)[C@@H](O)[C@H](O)[C@H]5O)c(NC(=O)CCNC(=O)[C@H](CCC(=O)O)NC(=O)[C@H](CCC(=O)O)NC(=O)CC)c3)CC4)cc2N=C(N)C1 ; CCc1ccc(N2C(=O)C=CC2=O)cc1C(=O)NCC(=O)NCC(=O)NCCOCCOCCOCCOCCOCCOCCOCCOCCOCCOCCOCCOCCC(=O)N[C@@H](CCC(=O)O)C(=O)O] 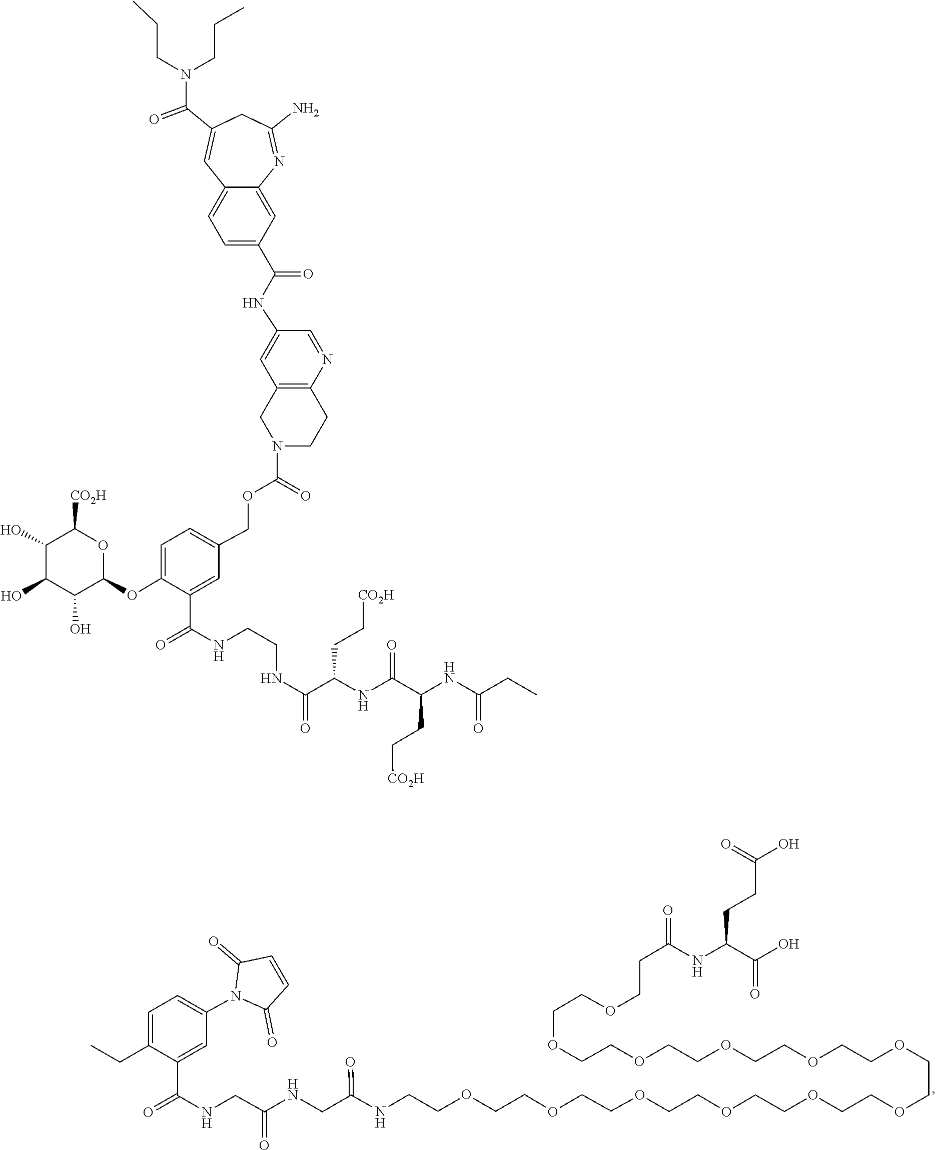 [SMILES: CCCN(CCC)C(=O)C1=Cc2ccc(C(=O)Nc3cnc4c(c3)CN(C(=O)OCc3ccc(O[C@@H]5O[C@H](C(=O)O)[C@@H](O)[C@H](O)[C@H]5O)c(C(=O)NCCNC(=O)[C@H](CCC(=O)O)NC(=O)[C@H](CCC(=O)O)NC(=O)CC)c3)CC4)cc2N=C(N)C1 ; CCc1ccc(N2C(=O)C=CC2=O)cc1C(=O)NCC(=O)NCC(=O)NCCOCCOCCOCCOCCOCCOCCOCCOCCOCCOCCOCCOCCC(=O)N[C@@H](CCC(=O)O)C(=O)O] 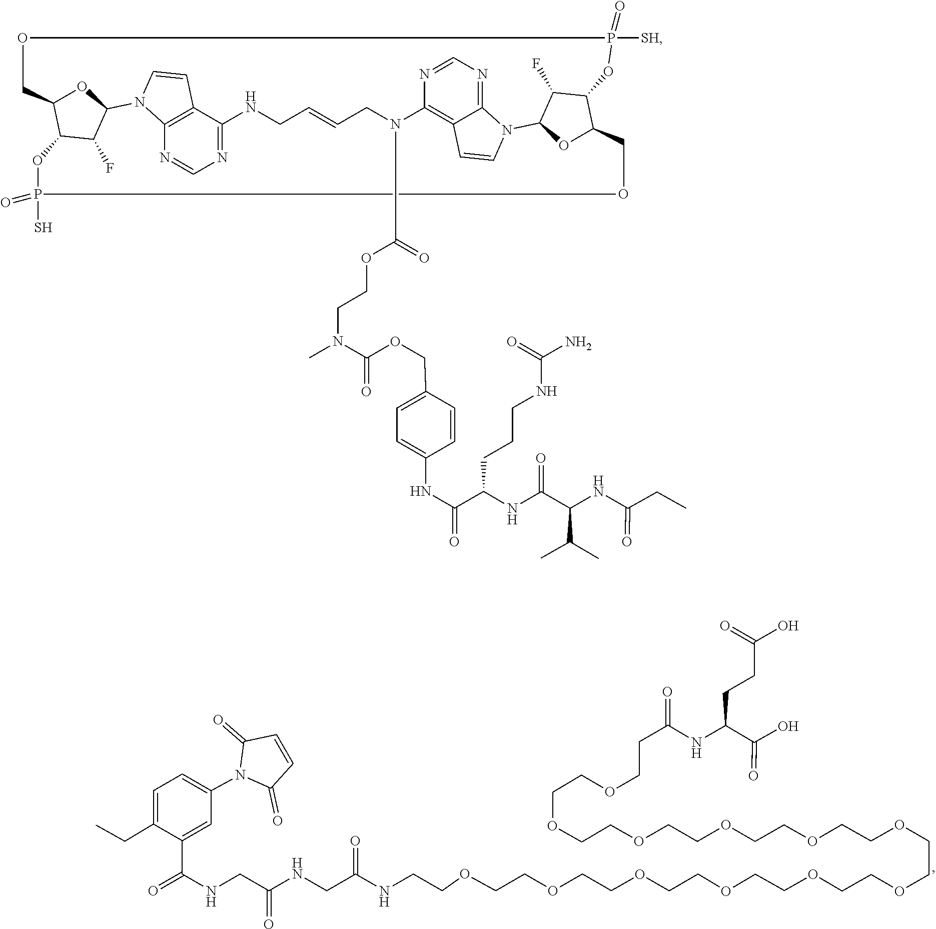 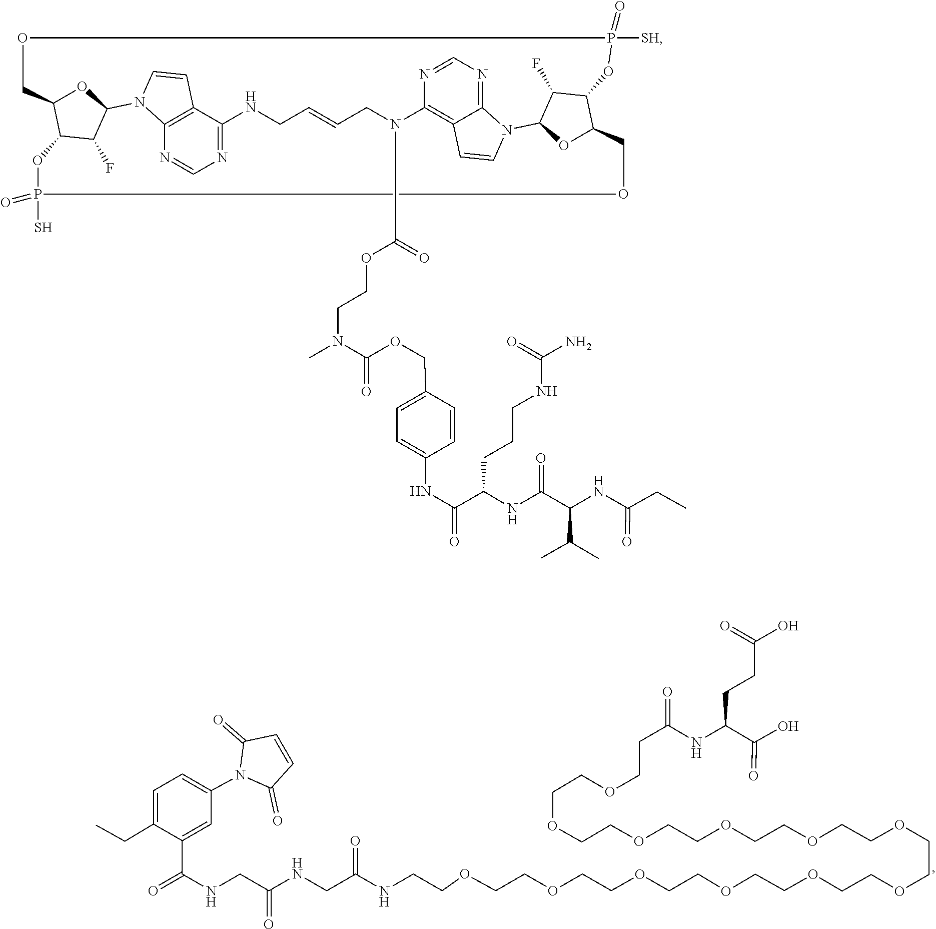 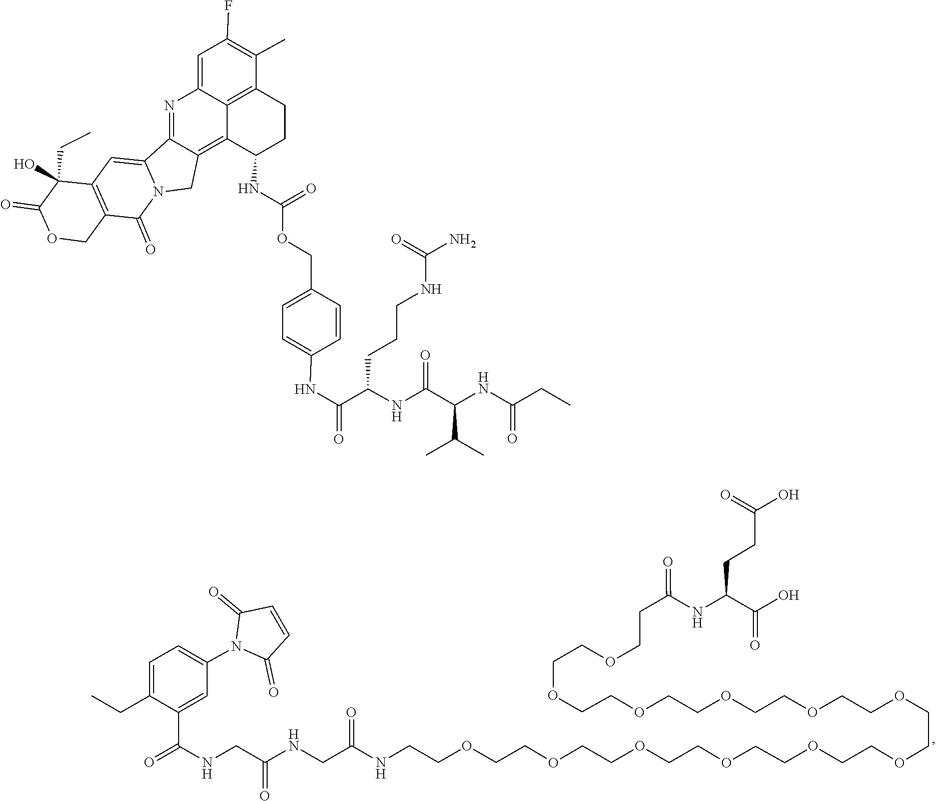 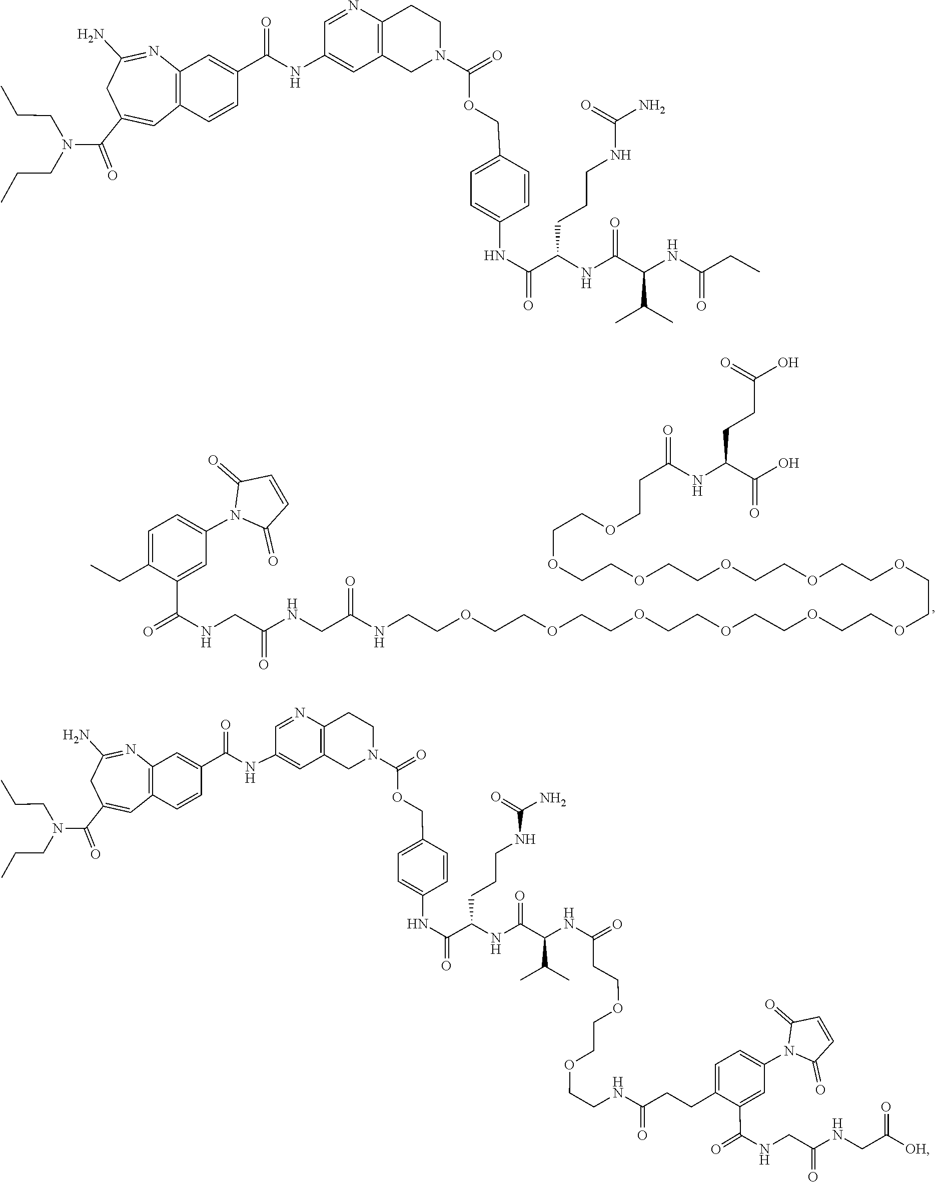 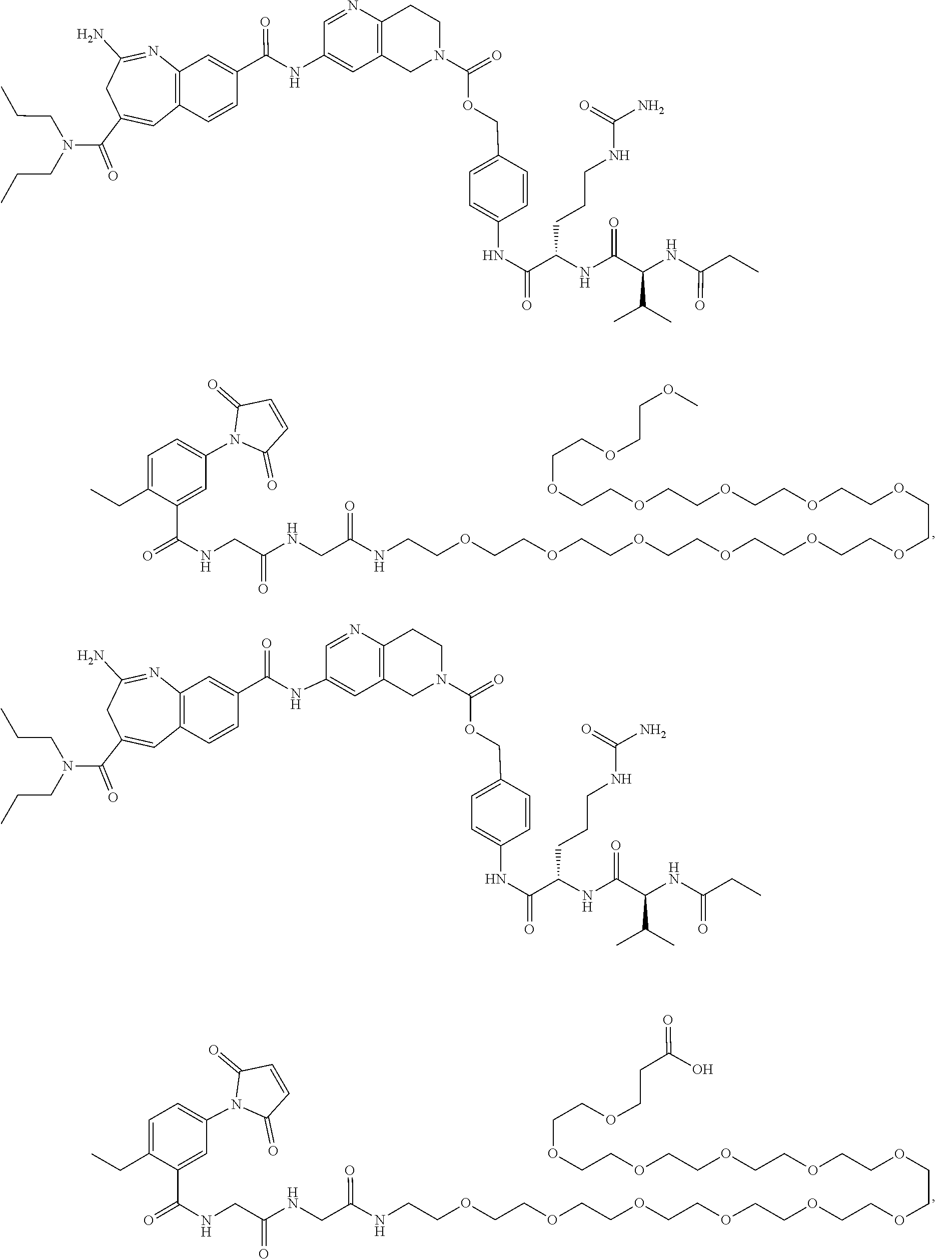 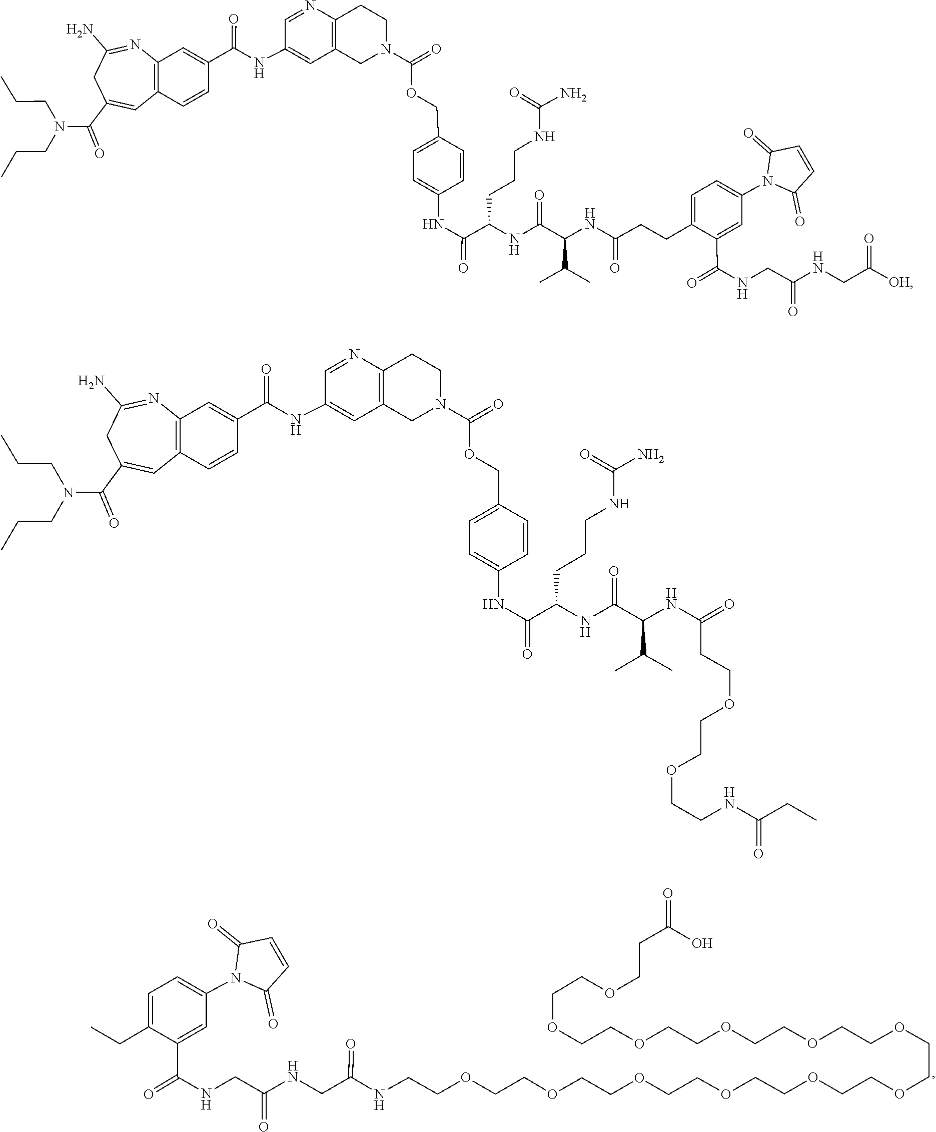 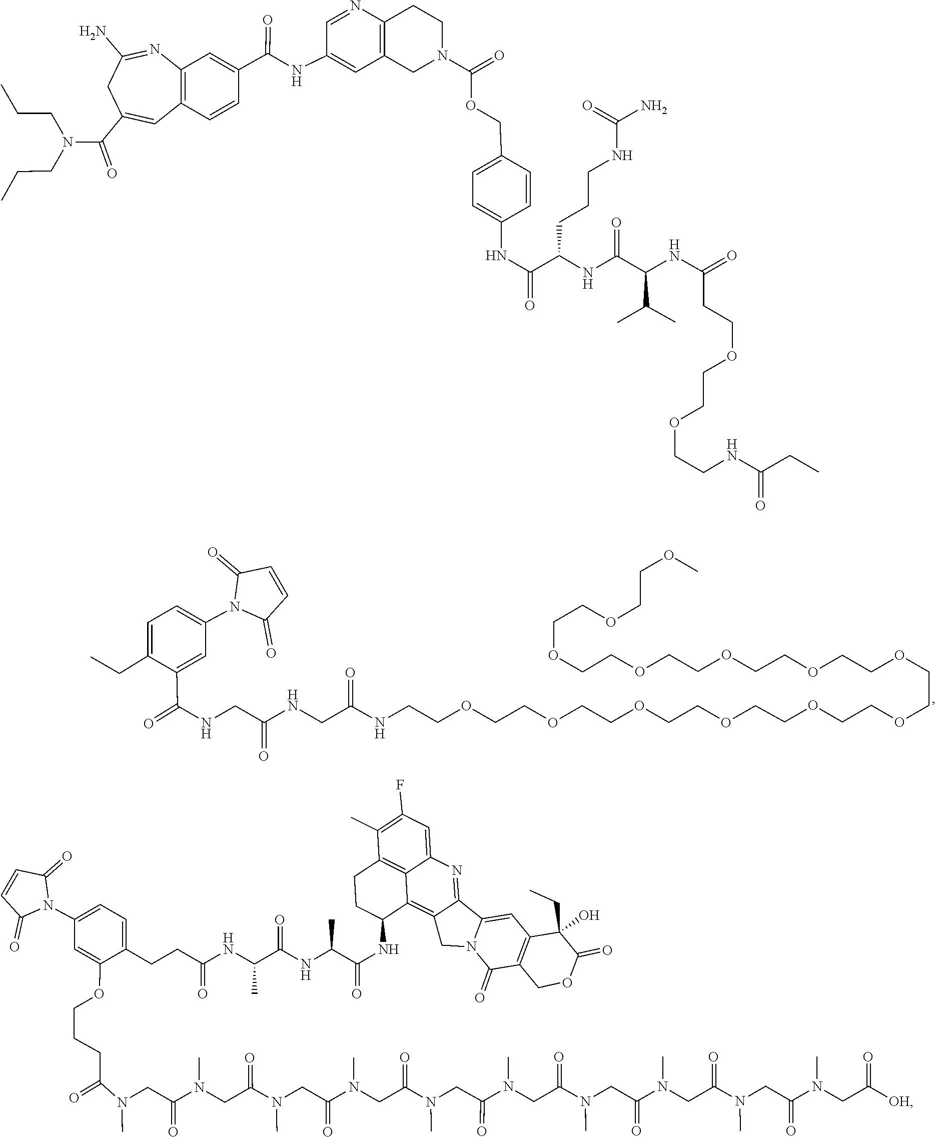 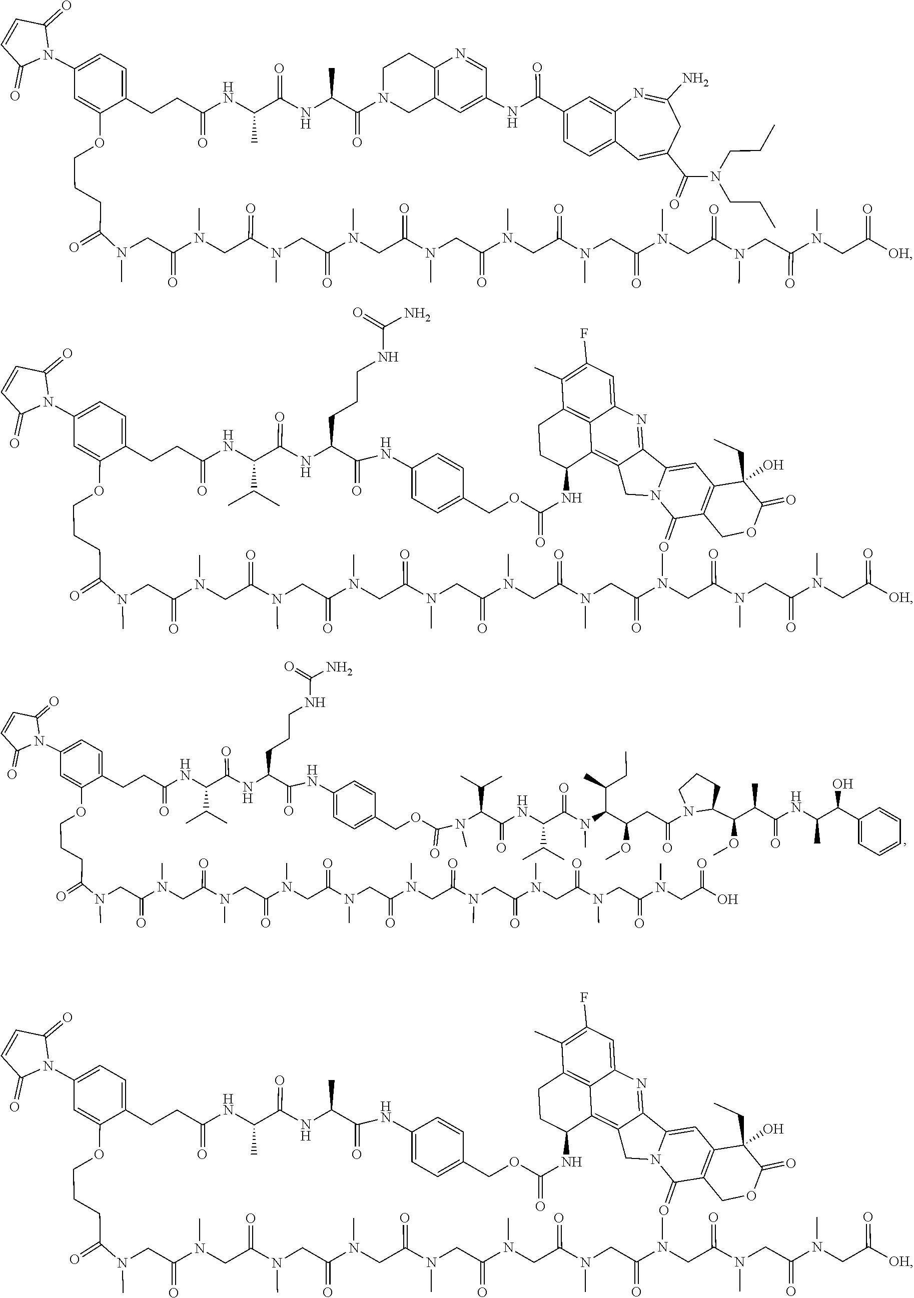 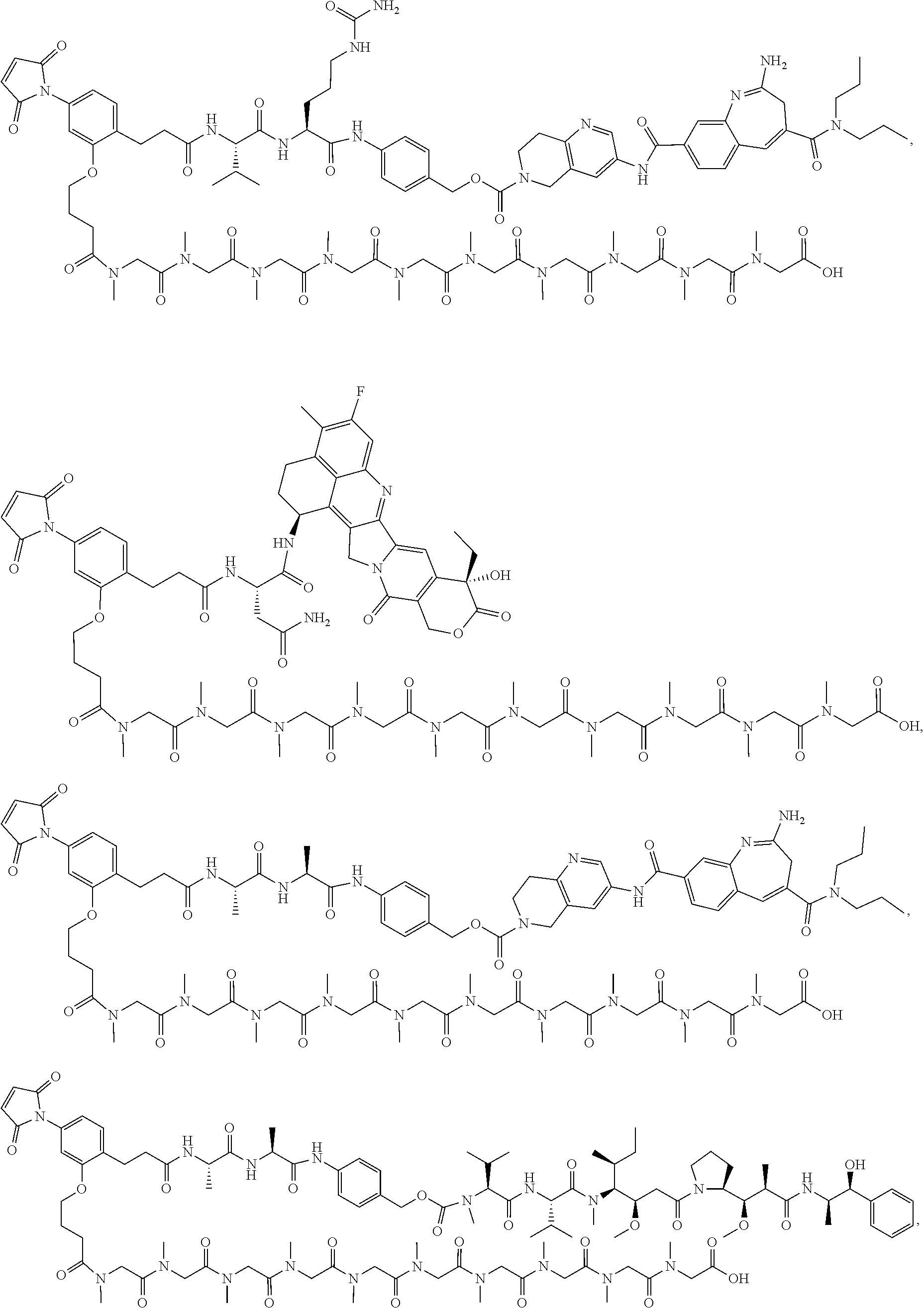 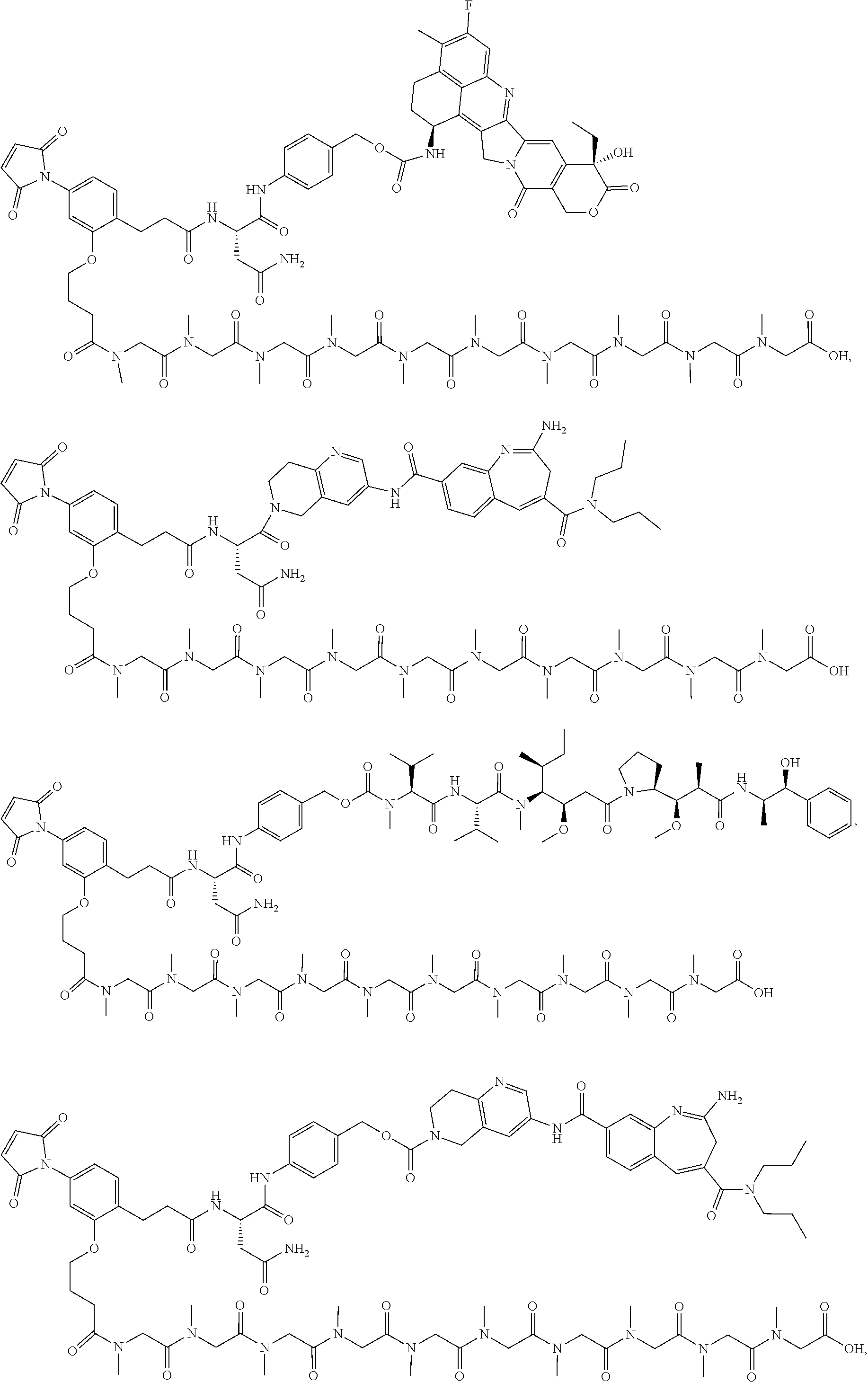 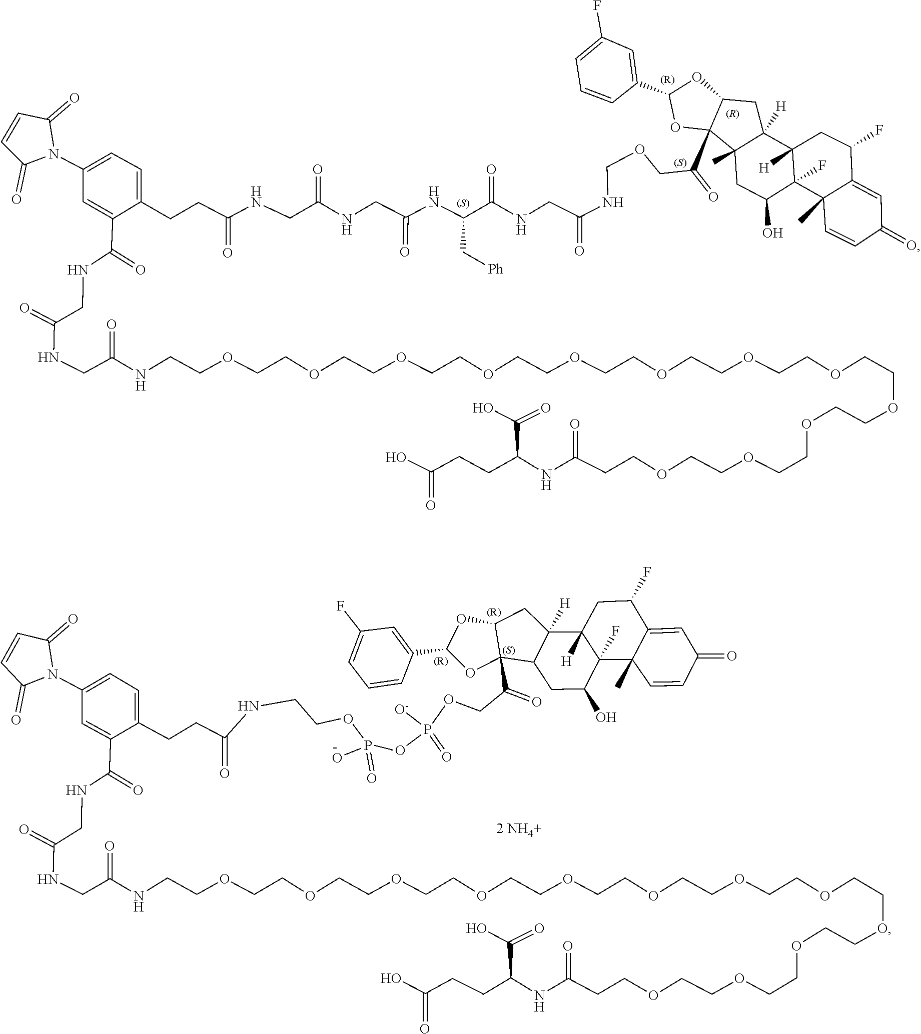 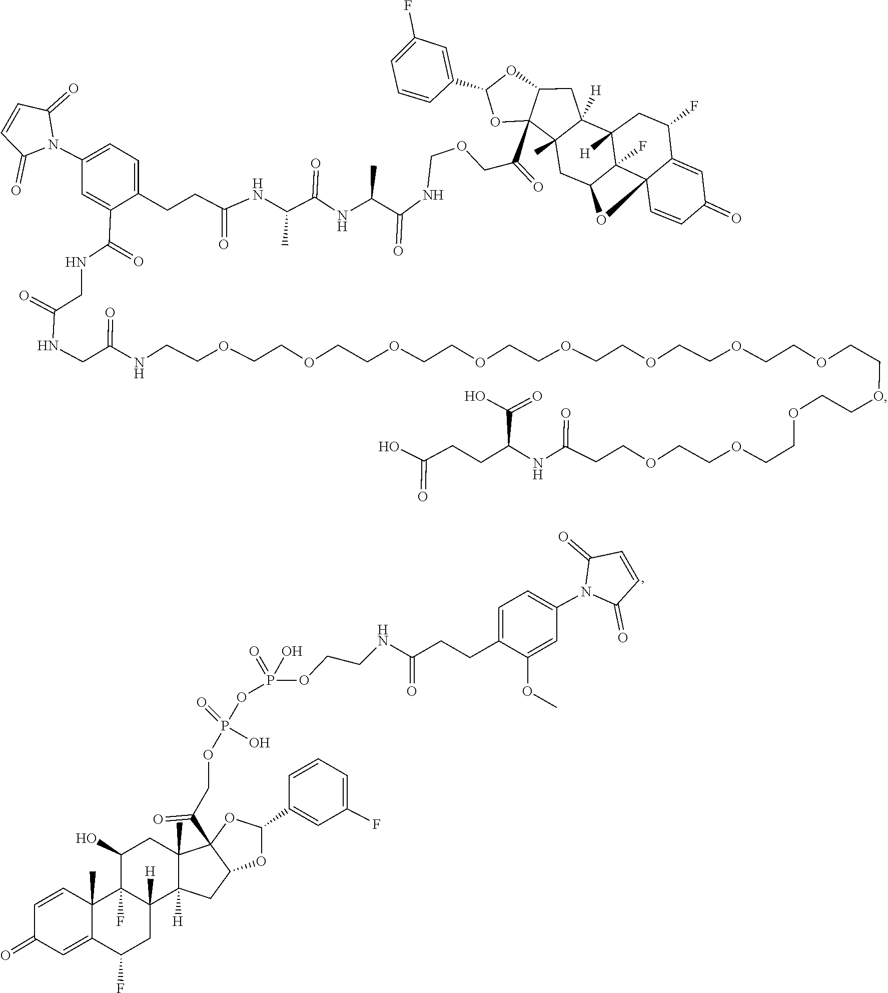 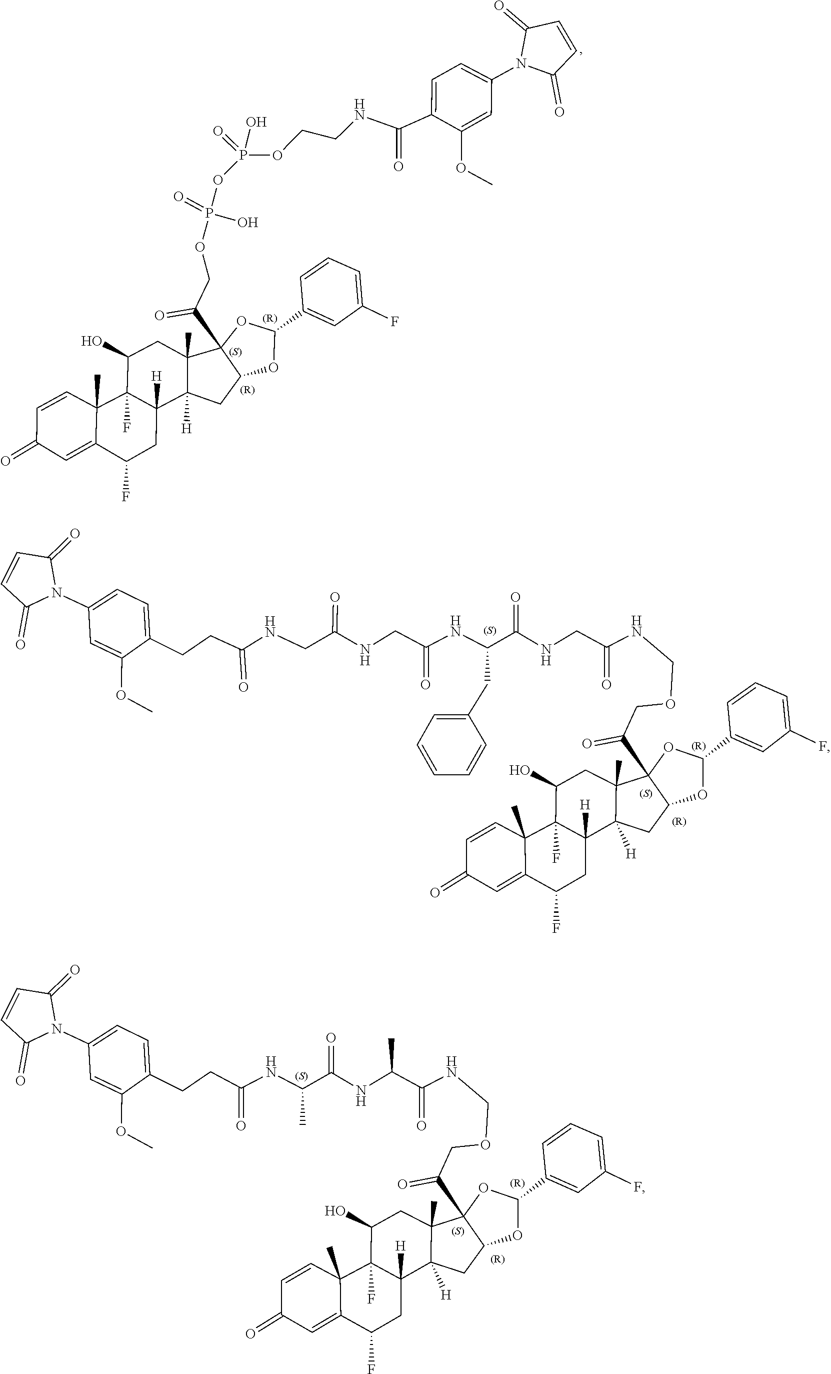 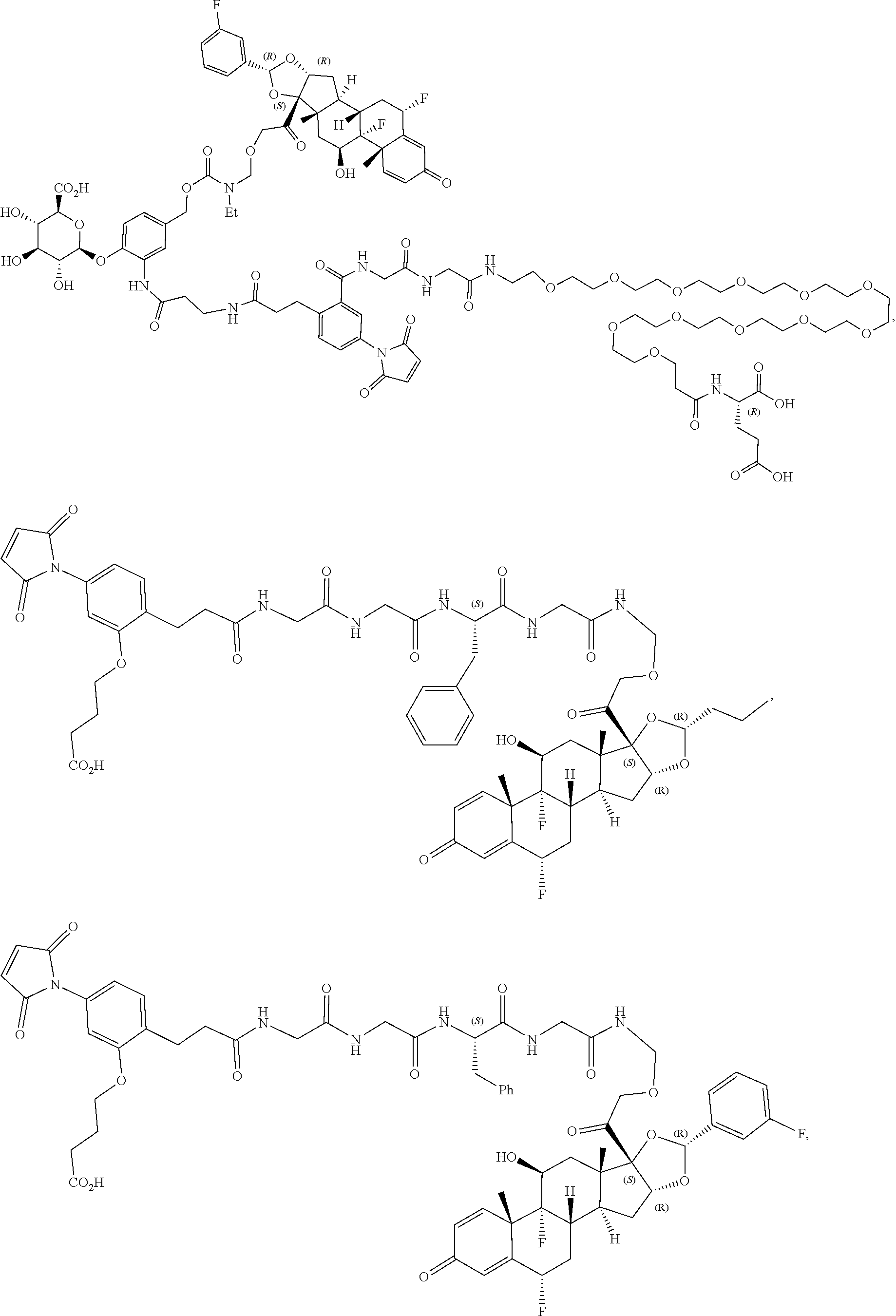 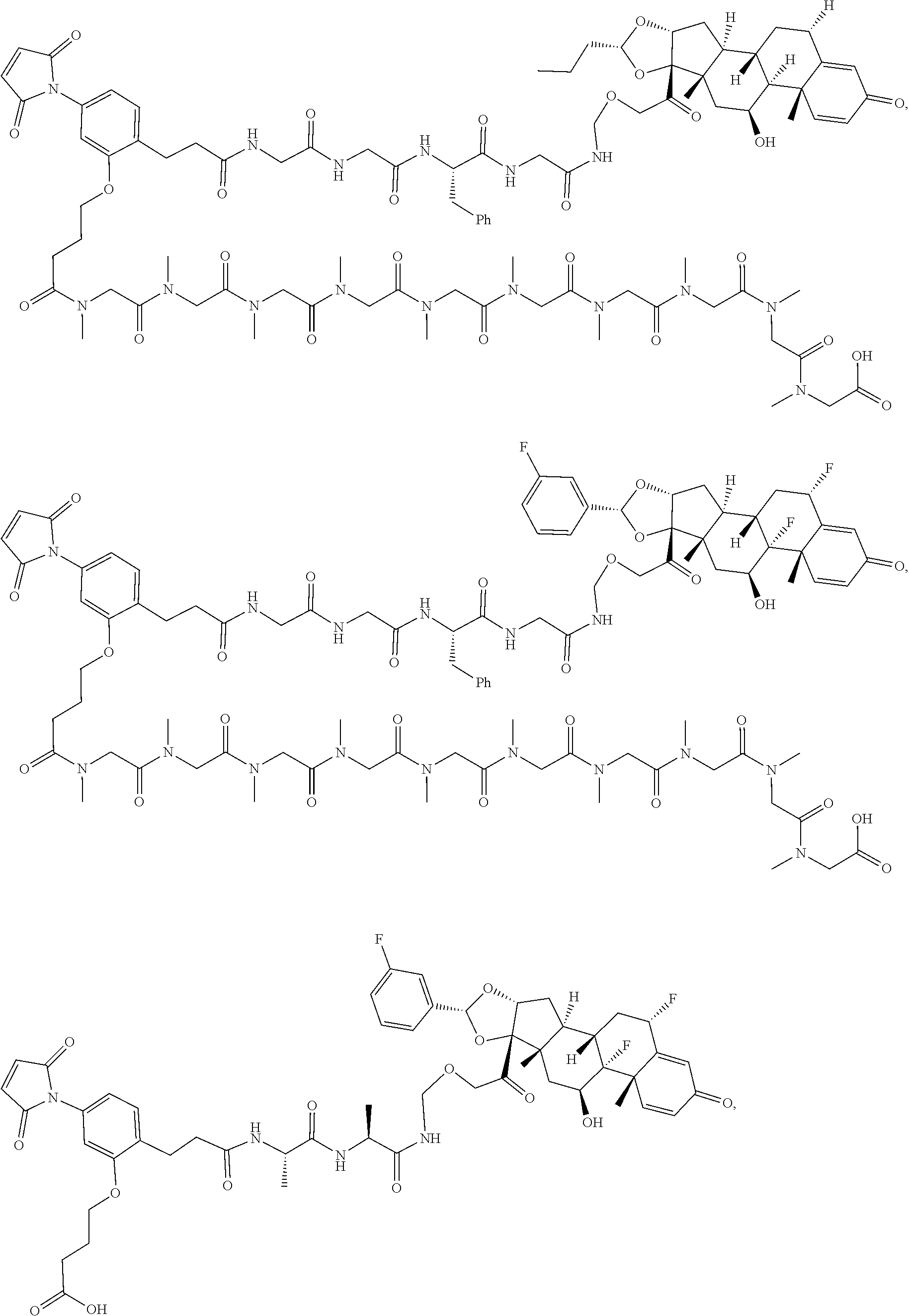 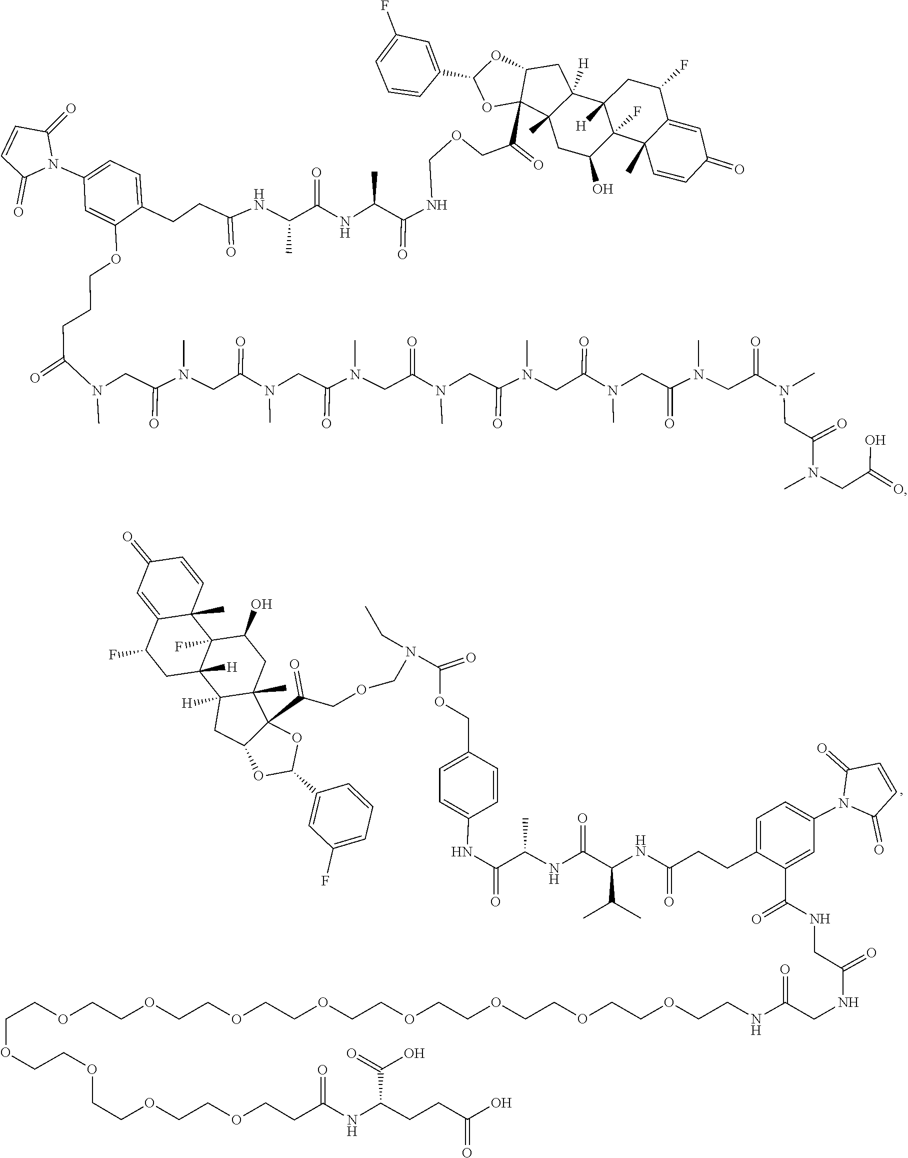 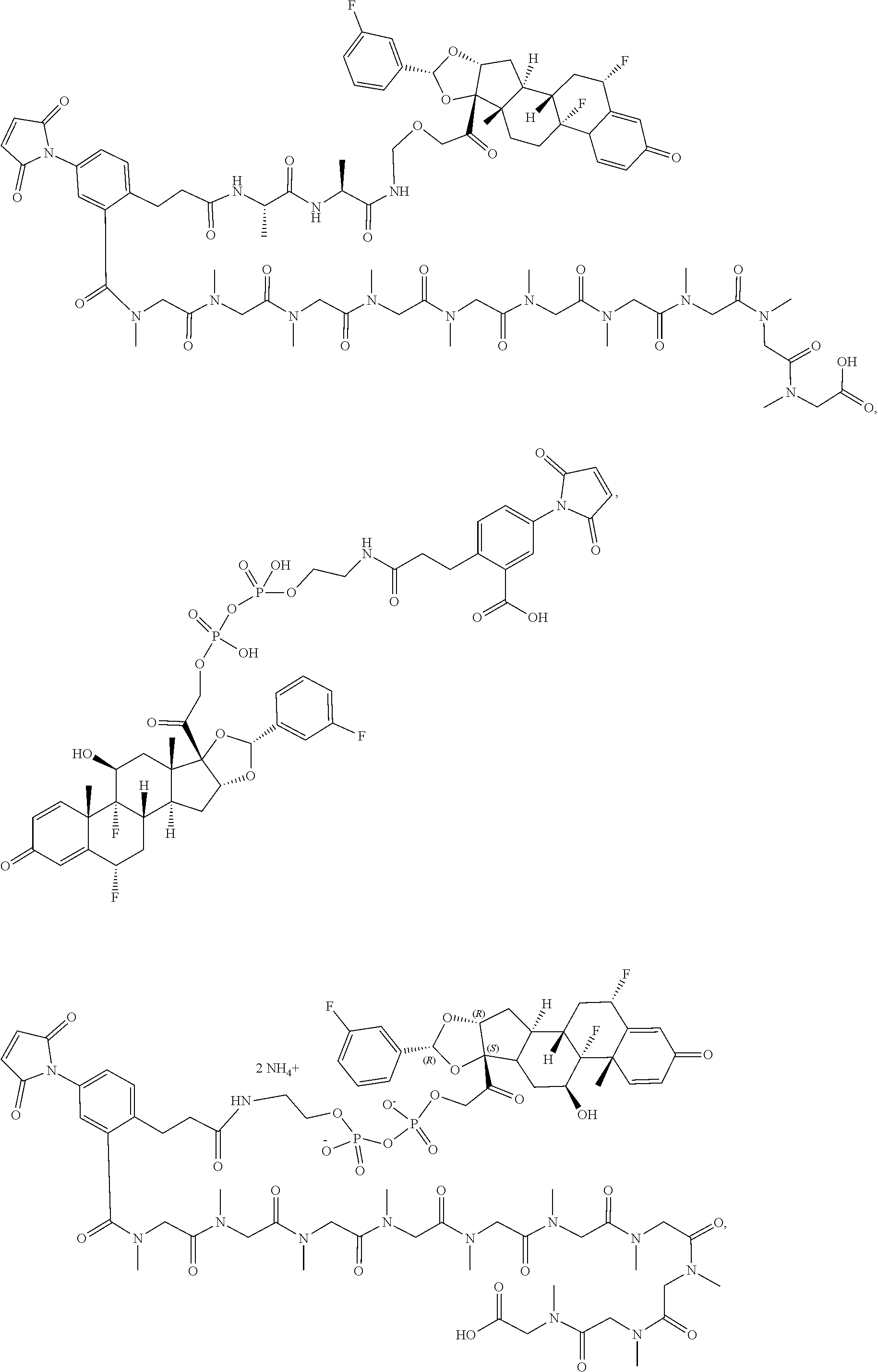 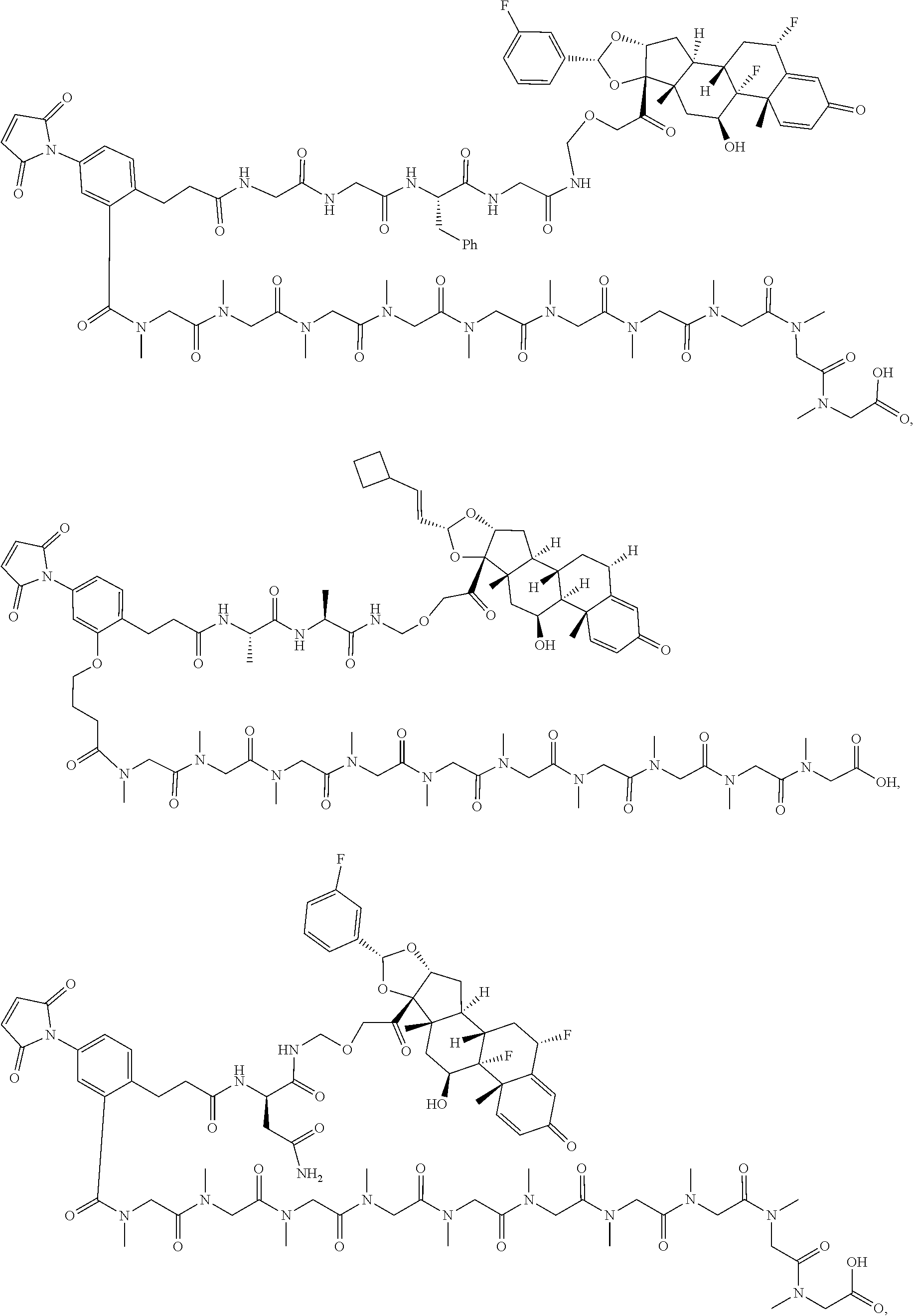 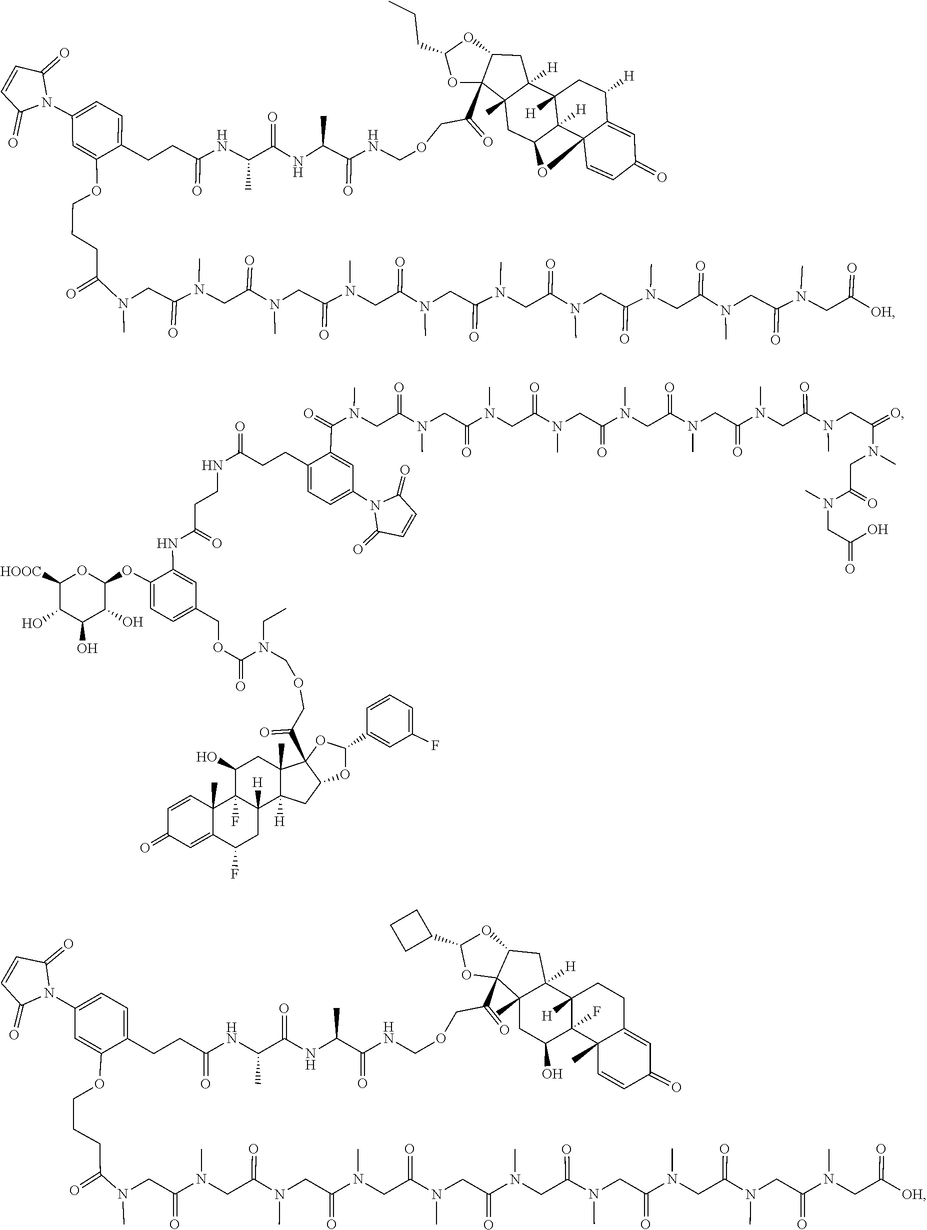 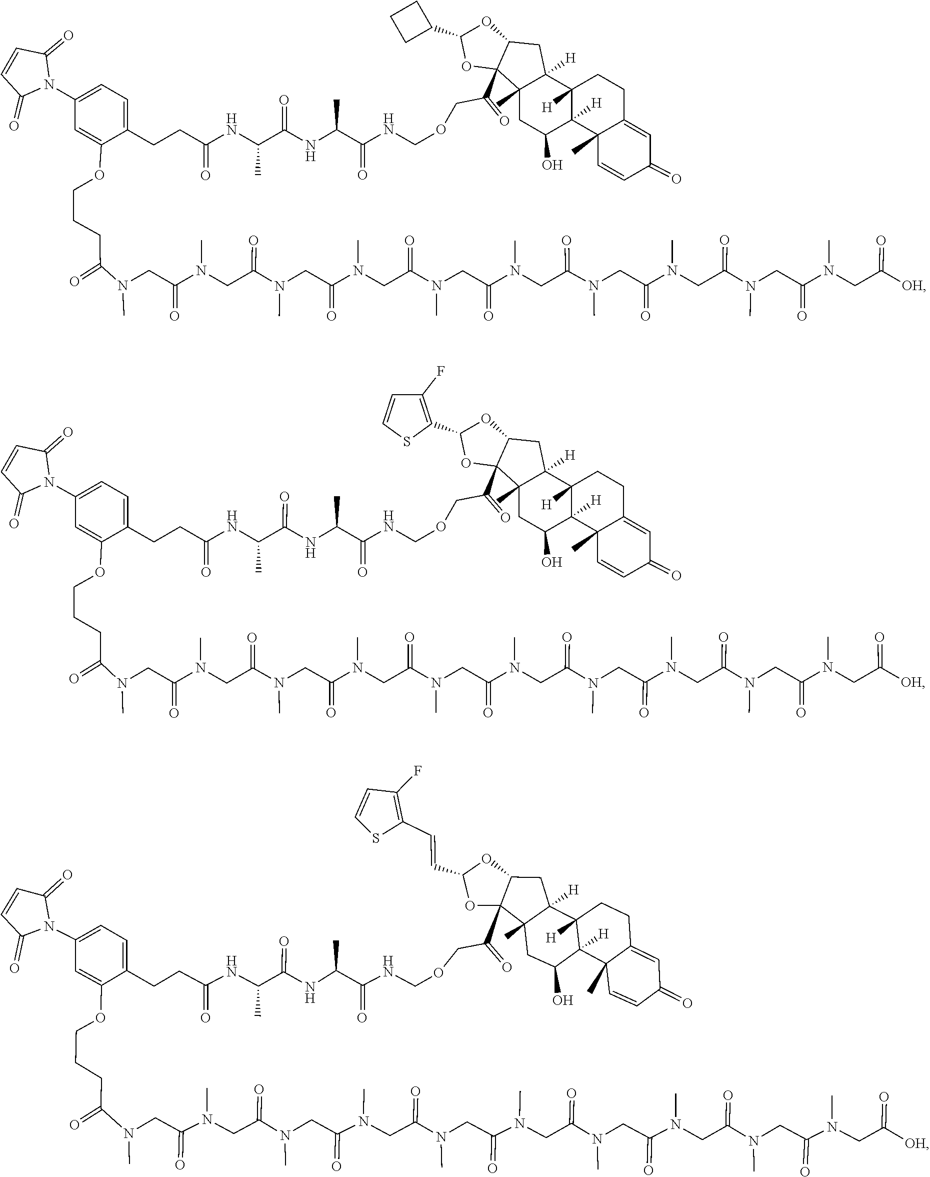 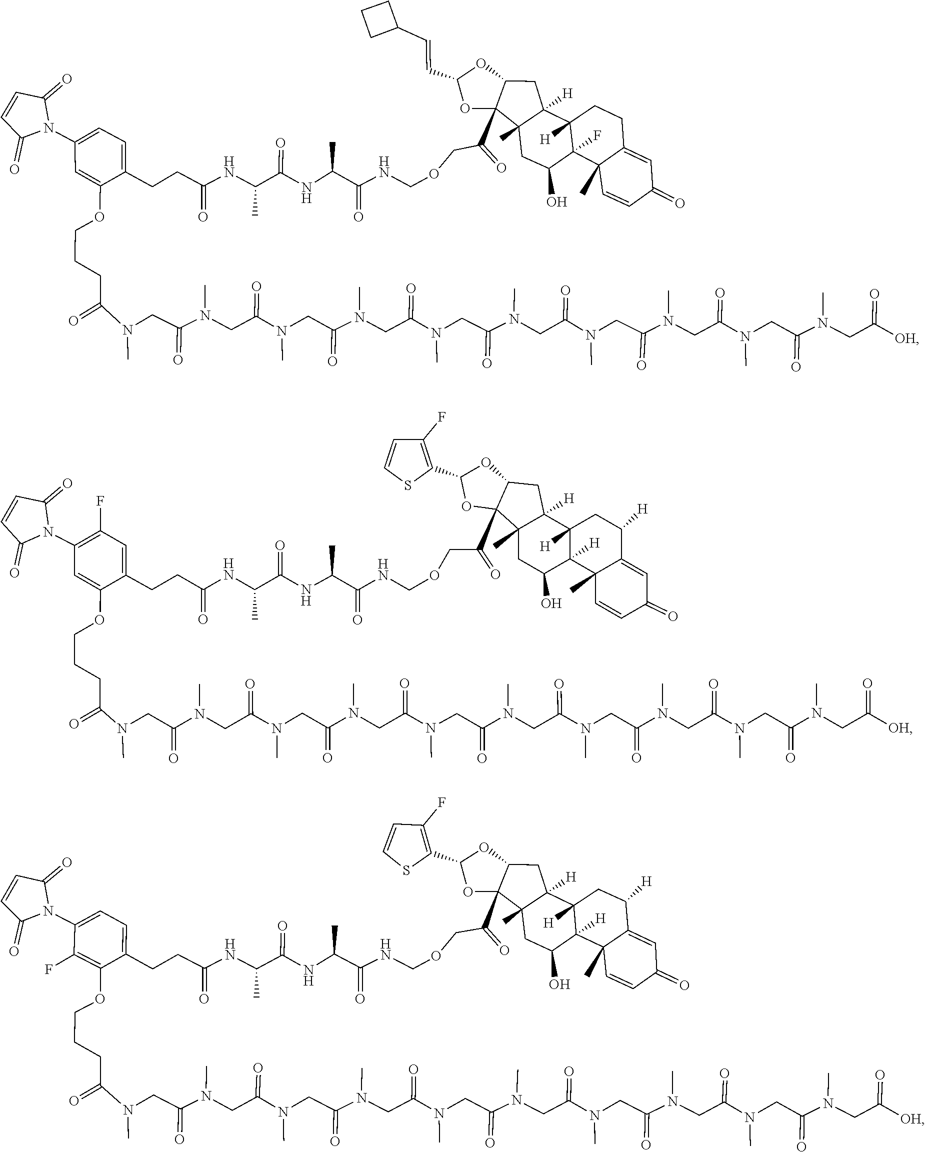 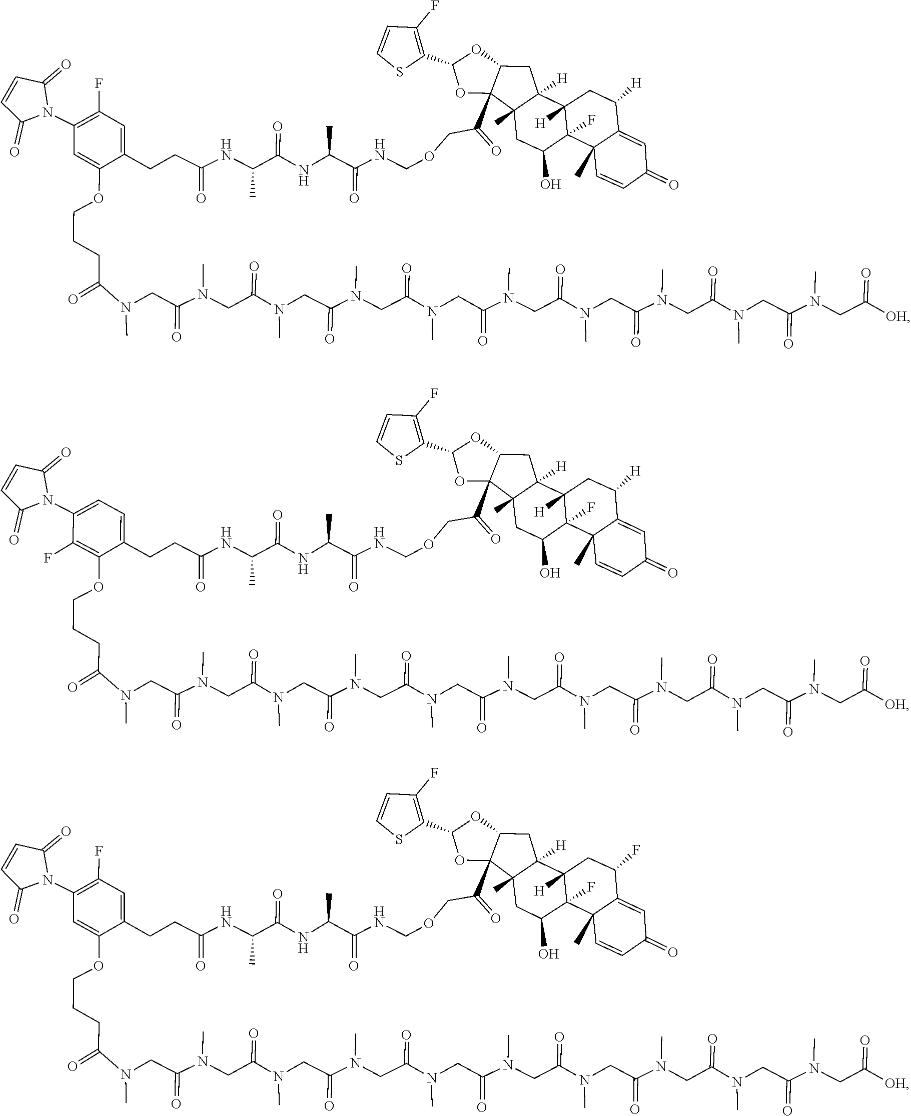 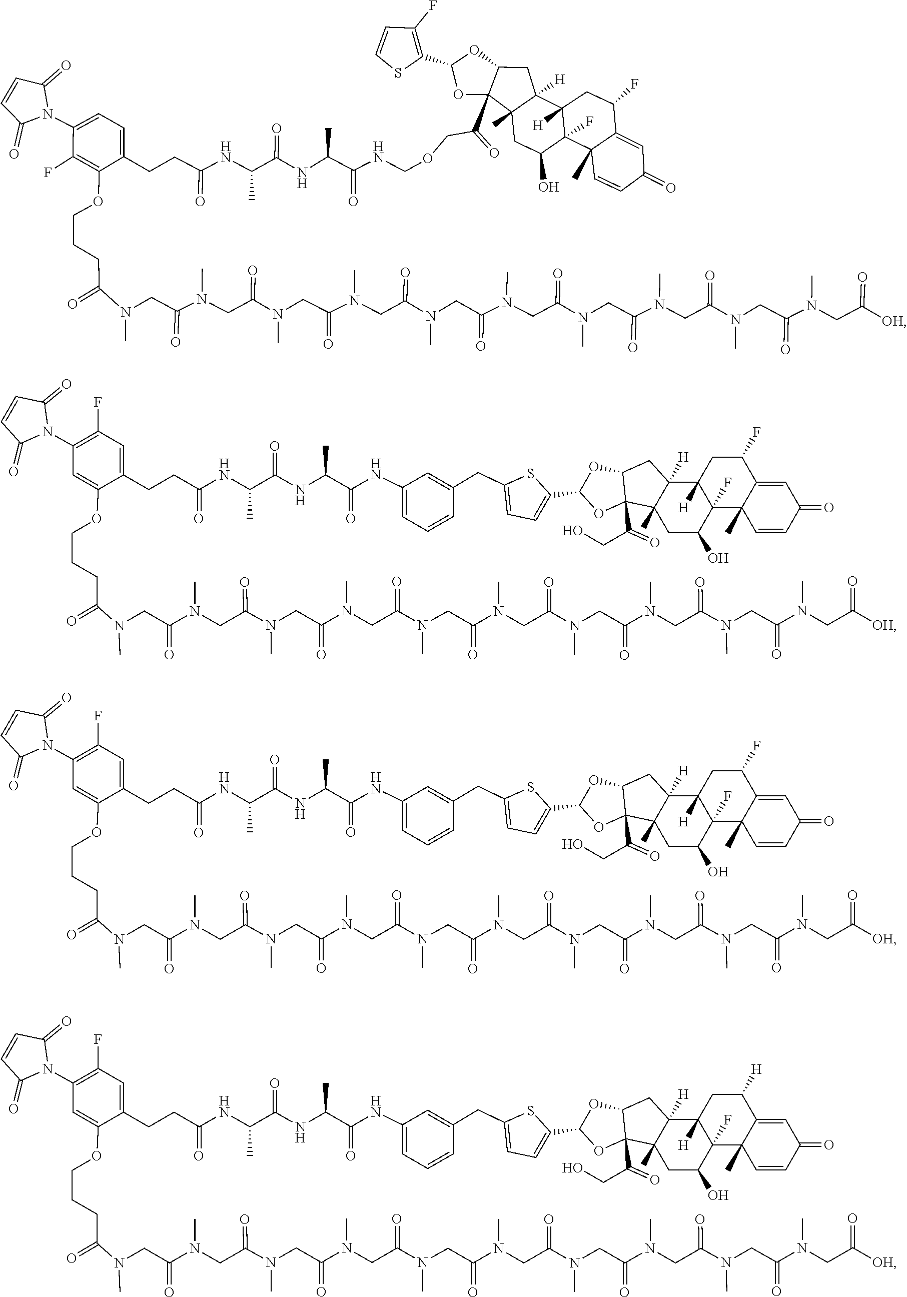 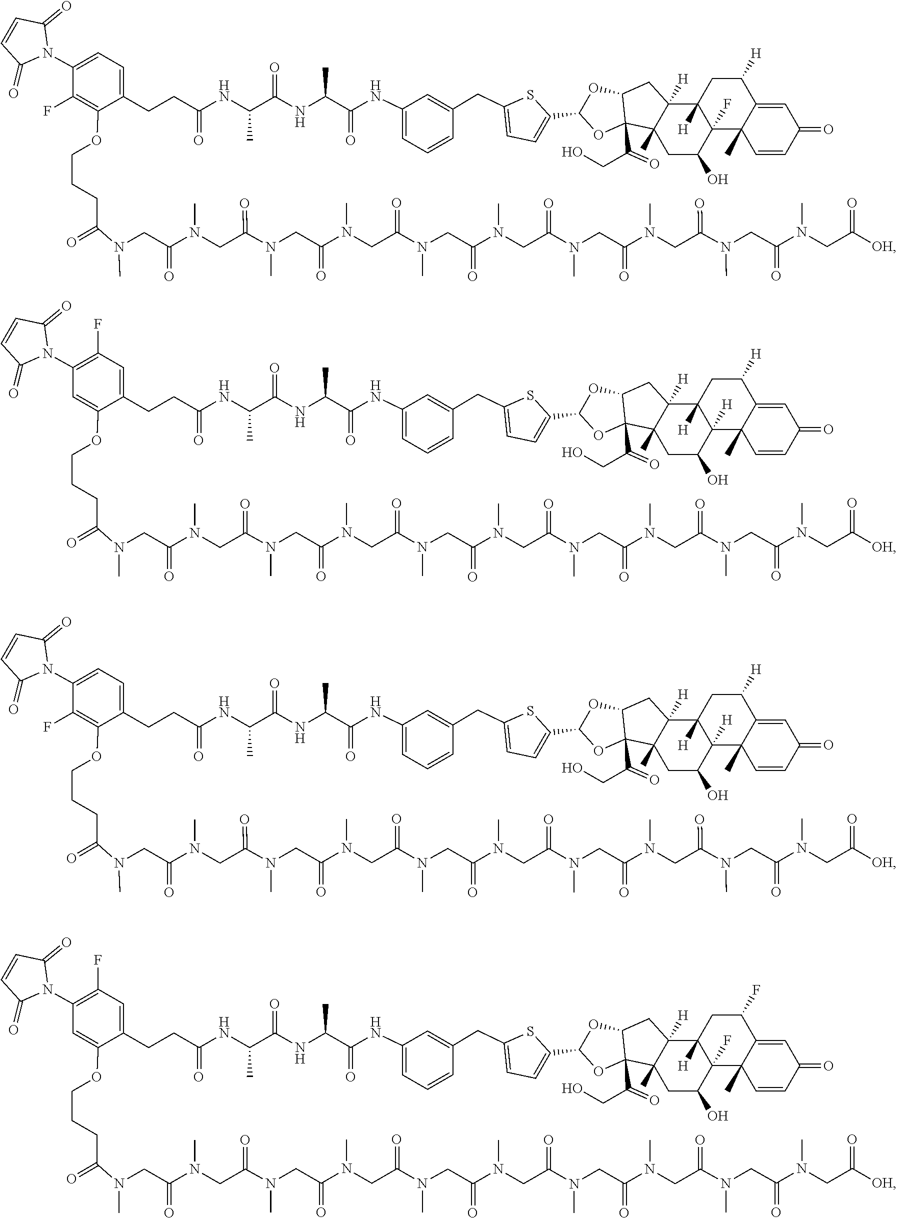 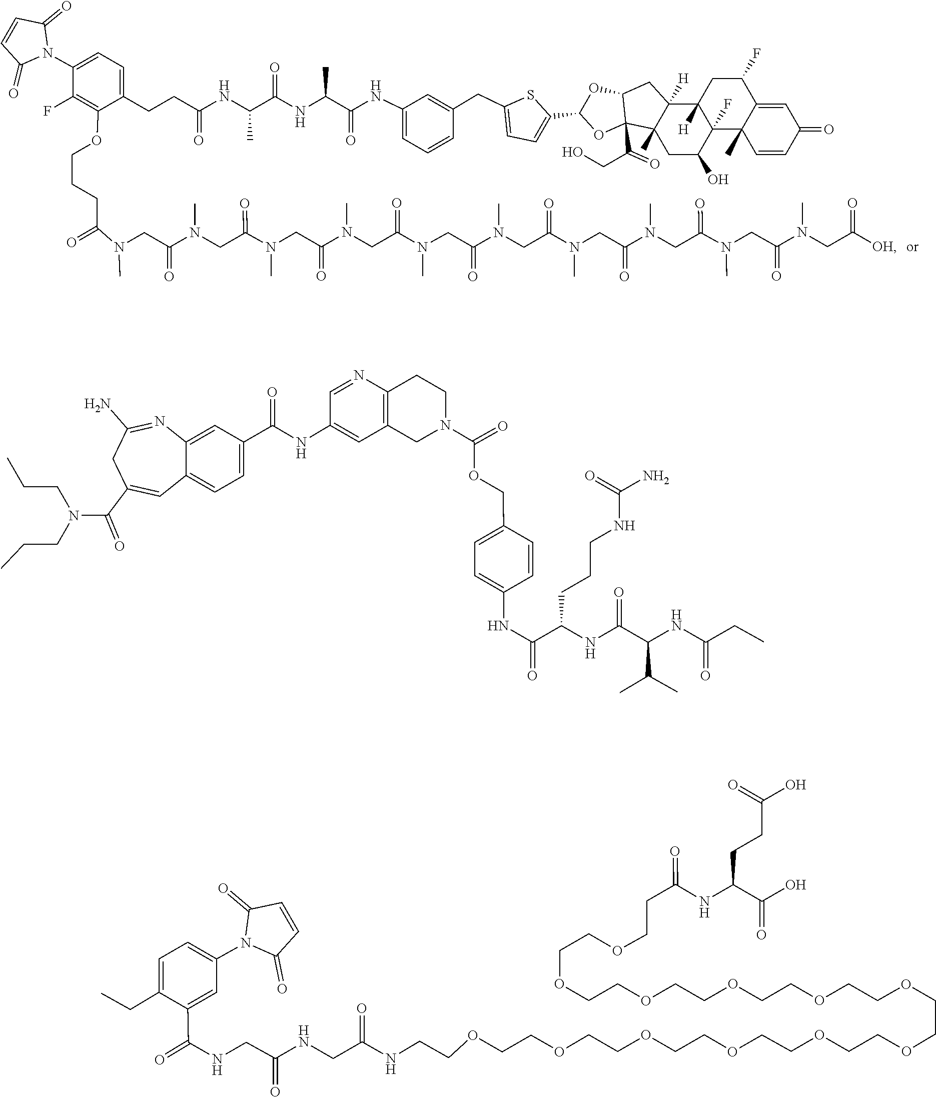
as a stereoisomer, enantiomer, or tautomer thereof or a mixture thereof; or a pharmaceutically acceptable salt, solvate or prodrug thereof.

158. A conjugate having the following Structure (IIa): 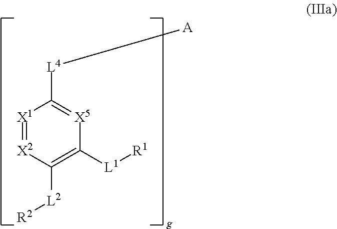
wherein:
A is a targeting moiety or a binding fragment thereof;
L⁴ has one of the following structures: 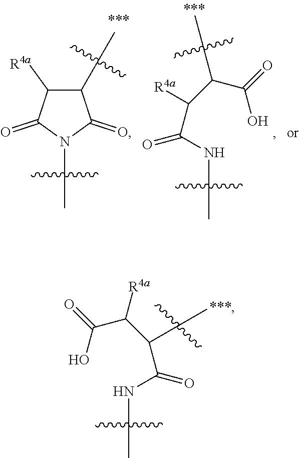
*** indicates an attachment point to A;
g is an integer from 1-20;
R¹ and R² each independently comprise one or more moieties selected from
i) an amino acid element that comprises 1, 2, 3, 4, or 5 amino acids selected from the group consisting of glycine, alanine, serine, threonine, cysteine, valine, leucine, isoleucine, methionine, proline, phenylalanine, tyrosine, tryptophan, aspartic acid, glutamic acid, asparagine, glutamine, histidine, lysine, arginine, sarconsine, and beta-alanine;
ii) a charged element comprising one or more charged amino acid, one or more carboxylic acid, one or more sulfonic acid, one or more sulfonamide, one or more sulfate, one or more phosphate, one or more quaternary amine, one or more sulfamide, one or more sulfinimide, or combinations thereof;
iii) a heteroalkylene element comprising one of the following structures: 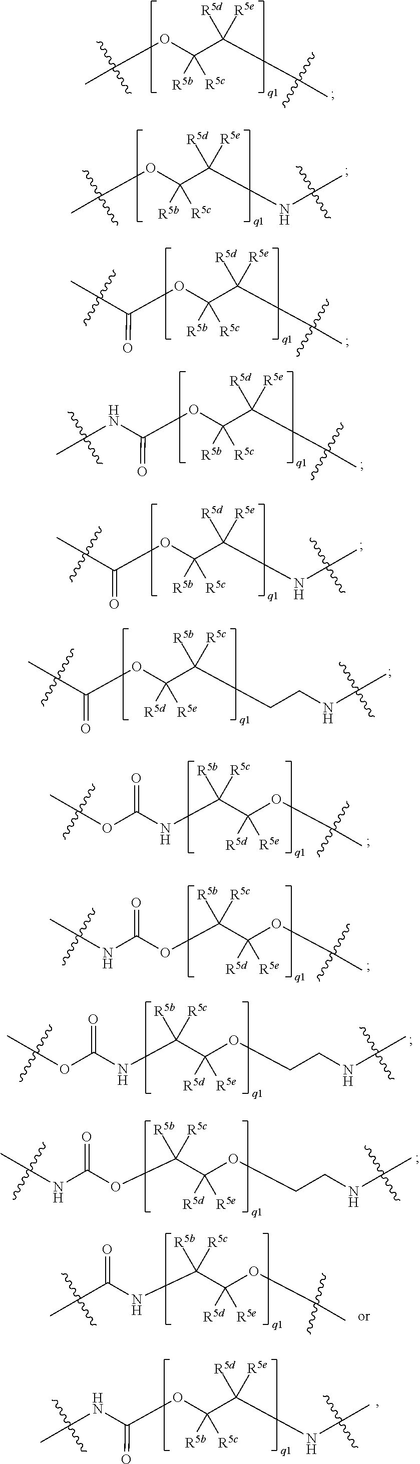
wherein
each occurrence of R^{5b}, R^{5c} R^{5d}, and R^{5e} is independently selected from the group consisting of hydrogen, deuterium, alkyl, haloalkyl, halo, alkoxy, haloalkoxy, amino, hydroxyl, cyano, nitro, thiol, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, and —P(O)₃H,
each occurrence of q1 is, independently an integer from 1-24;
iv) a hydrophilic element comprising polyethylene glycol, polysarcosine, cyclodextrin, c-glycosides, or combinations thereof;
v) a trigger element comprising two or more amino acids selected from the group consisting of valine, citrulline, alanine, glycine, phenylalanine, lysine, or combinations thereof;
vi) an immolative element comprising para-aminobenzyloxycarbonyl, an aminal, a hydrazine, a disulfide, an amide, an ester, a hydrazine, a phosphotriester, a diester, a β-glucuronide, a double bond, a triple bond, an ether bond, a ketone, a diol, a cyano, a nitro, a quaternary amine, or combinations thereof;
vii) a polar cap comprising —OH, —NH₂, —C(═O)OH, —S(O)₃, or —OP(O)₃;
viii) a payload that is a chemotherapeutic, a cytotoxic agent, or a myeloid cell agonist, provided that at least one of R¹ and R² comprises a payload;
R^{4a} is hydrogen, deuterium, halo, or —S—R^{4c} wherein R^{4c} is substituted or unsubstituted C₆-C₁₀ aryl or substituted or unsubstituted 5-12 membered heteroaryl;
L¹ and L² independently have one of the following structures: 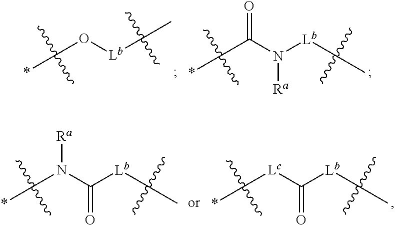
wherein:
* indicates a direct bond to the ring carbon as indicated in Structure (IIa);
R^{a} is hydrogen or alkyl;
each occurrence of L^{b} is independently a direct bond, an optionally substituted alkylene linker, an optionally substituted heteroalkylene linker, a heteroatomic linker, or combinations thereof;
each occurrence of L^{c} is independently an optionally substituted alkylene linker; and
X¹, X², and X⁵ are independently CH or CF, as a stereoisomer, enantiomer, or tautomer thereof or a mixture thereof; or a pharmaceutically acceptable salt, solvate or prodrug thereof.

159. A pharmaceutical composition comprising the conjugate of claim 158 and a pharmaceutically acceptable excipient.

160. A method for treatment of cancer comprising administering an effective amount of the conjugate of claim 158 to a subject in need thereof.

161. A method for treatment of infection, inflammation, an autoimmune disorder, or combinations thereof, the method comprising administering an effective amount of the claim 158 to a subject in need thereof.
